(19) United States
(12) Patent Application Publication
(10) Pub. No.: US 2026/0263423 A1
(43) Pub. Date: Sep. 10, 2026

(54) METHODS AND COMPOUNDS FOR MODULATING INHERITED GENETIC DISEASES

(71) Applicant: Design Therapeutics, Inc., Carlsbad, CA (US)

(72) Inventors: Chengzhi ZHANG, Carlsbad, CA (US); Abhijit BHAT, Carlsbad, CA (US); Santosh C. SINHA, Carlsbad, CA (US); Fei YANG, Carlsbad, CA (US)

(21) Appl. No.: 18/875,089

(22) PCT Filed: Jun. 14, 2023
(86) PCT No.: PCT/US2023/025329

§ 371 Date: Dec. 13, 2024

Related U.S. Application Data
(60) Provisional application No. 63/352,397, filed on Jun. 15, 2022.
(60) Provisional application No. 63/482,751, filed on Feb. 1, 2023.

(51) Int. Cl. A61K 31/4178; A61K 31/454; A61K 31/4545

(52) U.S. Cl. CPC A61K 31/4178; A61K 31/454; A61K 31/4545

(57) ABSTRACT

The present disclosure relates to transcription modulator molecule compounds, compositions, and methods of treating various genetic diseases including spinocerebellar ataxias and Huntington's disease.

### CROSS REFERENCE TO RELATED APPLICATIONS

This application claims the benefit of U.S. Application No. 63/352,397, filed Jun. 15, 2022, and U.S. Application No. 63/482,751, filed Feb. 1, 2023, both of which are hereby incorporated by reference in their entirety.

### FIELD OF THE DISCLOSURE

Disclosed herein are new chimeric heterocyclic polyamide compounds and compositions and their application as pharmaceuticals for the treatment of diseases. Methods to modulate the expression of a target gene comprising the CAG trinucleotide repeat sequence in a human or animal subject are also provided for the treatment of diseases such as spinocerebellar ataxia, Huntington's disease (“HD”), Huntington's disease-like syndrome, spinobulbar muscular atrophy, and dentatorubral-pallidoluysian atrophy.

### BACKGROUND OF THE DISCLOSURE

The disclosure relates to the treatment of inherited genetic diseases characterized by overproduction of mRNA.

Spinocerebellar ataxia refers to a family of genetic diseases that is characterized by neuronal degeneration, particularly in the cerebellum. Symptoms are generally related to loss of motor function, and include incoordination of gait, poor coordination of manual and eye movements, dysarthria (unclear speech) and related complications such as poor nutrition due to dysphagia.

Several subclasses of spinocerebellar ataxia (“SCA”) have been identified, of which a number are linked with the presence of oligonucleotide repeat sequences. Of these, several are associated with multiple copies of the CAG trinucleotide repeat sequence. In six of the subclasses: SCA1, SCA2, SCA3, SCA6, SCA7, and SCA17, the CAG trinucleotide repeat sequences give rise to polyglutamine (poly-Q) repeat sequences in the coded proteins. These subclasses of SCA, along with Huntington's disease, dentatorubral-pallidoluysian atrophy, and spinobulbar muscular atrophy, have been collectively termed “polyglutamine expansion disorders.” The exact mechanism that links the defective poly-Q proteins to the observed pathology is not always clear; aggregation of the protein, as well as formation of ubiquitinated inclusion bodies, has been proposed.

In contrast to the above group of diseases, the CAG trinucleotide repeat sequence of SCA12 is located outside of the coding region of the gene. Thus, although the mRNA contains the CAG trinucleotide repeat sequence, translation of the mRNA does not produce a poly-Q tract. The pathology associated with this defect may be due to the failure of normal cellular mechanisms to break down the abnormal mRNA, perhaps due to the presence of stable hairpin structures, leading to accumulation of the mRNA in the cell.

Spinocerebellar ataxia type 1 (“SCA1”) is associated with the presence of the CAG trinucleotide repeat sequence in the atxn1 gene. Afflicted individuals have 39 or more of the trinucleotide repeat sequence; age of onset of symptoms is inversely correlated with a higher count of trinucleotide repeat sequences. The condition is generally fatal within 10-30 years; no curative treatment is currently available. The CAG trinucleotide repeat sequence is observed in mRNA as well as in genomic DNA. The gene codes for a protein termed ATXN1 which contains a poly-Q tract from the CAG trinucleotide repeat sequences. Animal studies indicate that protein toxicity, and not loss of function, is the primary mechanism responsible for the pathology of defective ATXN1. Degradation of defective ATXN1 by the proteasome is impaired, leading to accumulation of the protein.

Spinocerebellar ataxia type 2 (“SCA2”) is associated with the presence of the CAG trinucleotide repeat sequence in the atxn2 gene. Afflicted individuals have 32 or more of the trinucleotide repeat sequences; age of onset of symptoms is inversely correlated with a higher count of trinucleotide repeat sequences. The gene codes for a protein termed ATXN2 which contains a poly-Q tract from the CAG trinucleotide repeat sequences. The function of the ATXN2 protein is not well understood: it is cytoplasmic and associated with Golgi bodies and the endoplasmic reticulum. Regulation of mRNA translation is suggested by the RNA binding property of ATXN2.

Spinocerebellar ataxia type 3 (“SCA3”) is associated with the presence of the CAG trinucleotide repeat sequence in the atxn3 gene. Afflicted individuals have 50 or more copies of the trinucleotide repeat sequence; age of onset of symptoms is inversely correlated with a higher count of trinucleotide repeat sequences. The gene codes for a protein termed ATXN3 which contains a poly-Q tract from the CAG trinucleotide repeat sequences. The ATXN3 protein plays a role in the ubiquitin/proteasome mechanism for the metabolism of proteins: after a protein is marked for metabolism by ubiquitination, and before degradation of the protein by the proteasome, ATXN3 removes the ubiquitin for recycling. Defective ATXN3 containing a poly-Q tract loses this catalytic property, thus leading to a build-up of unwanted proteins.

Spinocerebellar ataxia type 6 (“SCA6”) is associated with the presence of the CAG trinucleotide repeat sequence in the cacna1a gene. Afflicted individuals have 20 or more of the trinucleotide repeat sequences. Average onset of symptoms is 45 years; the disease progresses slowly, and the duration of the disease can span over 25 years. Treatment for the disease is supportive, with acetazolamide providing relief from ataxia. The gene codes for the alpha-1 subunit of the CaV2.1 calcium channel, which is essential for proper neuronal function. The alpha-1 subunit produced by the defective cacna1a gene in afflicted individuals migrates to the cytoplasm as well as the cell membrane, where it forms aggregates. The mechanism that leads to the observed symptoms is unclear, although malfunction of the calcium channel is suspected, as well as the formation of a toxic C-terminal segment from posttranslational cleavage of the expanded protein.

Spinocerebellar ataxia type 7 (“SCA7”) is associated with the presence of the CAG trinucleotide repeat sequence in the atxn7 gene. Afflicted individuals have from 36 to over 300 of the trinucleotide repeat sequences. Onset of symptoms is typically observed in the second through fourth decade, with earlier onset correlating with more severe symptoms. In addition to the symptoms observed for the SCA class of diseases, subjects with SCA7, particularly subjects with earlier onset, can experience degradation of vision and blindness. Treatment for the disease is supportive only. The gene codes for the ataxin-7 protein, a nuclear protein that plays a role in transcription. In addition, the defective gene product interferes with cone-rod homeobox protein, providing an explanation for the retinopathy observed for this syndrome. Proteolytic cleavage of mutant ataxin-7 and transneuronal responses may be responsible for the pathogenesis of SCA7.

Spinocerebellar ataxia type 17 (“SCA17”) is associated with the presence of the CAG trinucleotide repeat sequence, with CAA interruptions, in the TATA box-binding protein (TBP) gene. The TBP gene product plays a role in the initiation of transcription. Afflicted individuals typically have 47 or more of the trinucleotide repeat sequences. Onset of symptoms is typically observed by age 50, with dysphagia often leading to aspiration and death. The link between the expanded CAG sequence and the observed pathology is unclear, with both gain-of-function and loss-of-function being suggested at different repeat lengths.

Huntington's disease (“HD”) was first identified in the late 19^{th} century as an autosomal dominant neurodegenerative disorder. The symptoms of HD, which include a range of movement, cognitive and psychiatric disorders, generally appear in adulthood. HD is associated with the presence of the CAG trinucleotide repeat sequence in the htt gene, which codes for a protein termed huntingtin. Subjects with more than about 36 trinucleotide repeat sequences generally present with symptoms of HD, with a larger number of trinucleotide repeat sequences associated with an earlier onset of symptoms. Pathology stems from a cascade of steps: production of poly-Q huntingtin, followed by fragmentation of the elongated huntingtin into smaller peptides, which bind together and accumulate in neurons. The effects of this cascade are pronounced in the basal ganglia and cortex of the brain.

Huntington's disease-like syndrome refers to a group of ailments whose symptoms are similar to those of Huntington's disease, but which lack the characteristic mutation in the htt gene. Huntington's disease-like 2 syndrome (“HDL2”) is associated with count of about 40 or more CAG trinucleotide repeat sequences in the junctophilin 3 (jph3) gene. HDL2 is a genetic disorder that has been seen in subjects with African lineage. Age of onset is inversely corelated with the number of trinucleotide repeat sequences. Symptoms of this syndrome include dystonia and chorea (uncontrolled movements), emotional disruptions, dysarthria, bradykinesia, inability to incorporate new learning, and difficulty in making decisions. Life expectancy can range from a few years post diagnosis to over a decade. The current theory holds that a poly-Q protein that is coded by the jph3 gene forms aggregates in neuronal cells that is responsible for the pathology of the disease. However, evidence suggesting toxic gain-of-function of mRNA has also been uncovered, indicating a possible dual pathway for pathology.

Spinobulbar muscular atrophy, also known as Kennedy disease, is an X-linked genetic disease observed in males whose symptoms include muscle atrophy, dysarthria and dysphagia due to bulbar muscles in the face and throat, fasciculations (involuntary twitches), and infertility. This disease is linked to the presence of the CAG trinucleotide repeat sequences in the androgen receptor (ar) gene. Pathology is thought to be due to the accumulation of fragments of the androgen receptor protein in nerve cells of the brain and spinal cord. Treatment is limited to management of symptoms; neither anti-androgen drugs nor testosterone or analogues display efficacy. Recent studies suggest that pathology of the poly-Q androgen receptor is due to inhibition of the ubiquitin ligase anaphase-promoting complex/cyclosome (APC/C), followed by disruptions in neurite formation and in the cell cycle.

Dentatorubral-pallidoluysian atrophy (“DRPLA”) is an autosomal dominant genetic disorder observed mostly in subjects of Japanese descent whose symptoms, which typically appear in adulthood, include seizures, ataxia, and myoclonus (involuntary spasmodic muscular contractions). Also observed in adult subjects are dementia and psychiatric disorders. DRPLA is linked with a CAG trinucleotide repeat sequence in the atrophin-1 (atn1) gene. Healthy individuals have fewer than about 34 trinucleotide repeat sequences; afflicted individuals generally have more than about 50 trinucleotide repeat sequences. The atrophin-1 protein (“ATN1”) is expressed in all tissue, but is proteolytically cleaved in neurons, suggesting a role in neural activity. Toxicity is thought to be due to accumulation of the ATN1 protein.

In some embodiments, the methods provide an effective treatment for a disease or disorder which is characterized by the presence of an excessive count of CAG trinucleotide repeat sequences in a target gene. In some embodiments, the pathology of the disease or disorder is due to the presence of mRNA containing an excessive count of CAG trinucleotide repeat sequences. In some embodiments, the pathology of the disease or disorder is due to the presence of a translation product containing an excessive count of glutamine amino acid residues. In some embodiments, the pathology of the disease or disorder is due to a loss of function in the translation product. In some embodiments, the pathology of the disease or disorder is due to a gain of function in the translation product. In some embodiments, the pathology of the disease or disorder can be alleviated by increasing the rate of transcription of the defective gene. In some embodiments, the pathology of the disease or disorder can be alleviated by decreasing the rate of transcription of the defective gene.

### SUMMARY OF THE DISCLOSURE

This disclosure utilizes regulatory molecules present in cell nuclei that control gene expression. Eukaryotic cells provide several mechanisms for controlling gene replication, transcription, and/or translation. Regulatory molecules that are produced by various biochemical mechanisms within the cell can modulate the various processes involved in the conversion of genetic information to cellular components. Several regulatory molecules are known to modulate the production of mRNA and, if directed to the target gene (for example, atxn1, atxn2, atxn3, cacna1a, atxn7, ppp2r2b, tbp, htt, jph3, ar, or atn1), would modulate the production of the target gene mRNA that causes diseases such as, for example, spinocerebellar ataxia, Huntington's disease, Huntington's disease-like syndrome, spinobulbar muscular atrophy, and dentatorubral-pallidoluysian atrophy; and thus reverse the progress of these diseases.

The disclosure provides compounds and methods for recruiting a regulatory molecule into close proximity to the target gene comprising a CAG trinucleotide repeat sequence. The compounds disclosed herein contain a DNA binding moiety that will selectively bind to the target gene. The DNA binding moiety will bind selectively the characteristic CAG trinucleotide repeat sequence of atxn1, atxn2, atxn3, cacna1a, atxn7, ppp2r2b, tbp, htt, jph3, ar, or atn1.

The mechanism provides an effective treatment for spinocerebellar ataxia, Huntington's disease, Huntington's disease-like syndrome. spinobulbar muscular atrophy, and dentatorubral-pallidoluysian atrophy, which are caused by the expression of defective atxn1, atxn2, atxn3, cacna1a, atxn7, ppp2r2b, tbp, htt, jph3, ar, and/or atn1. Correction of the expression of the defective target gene thus represents an effective method for the treatment for these diseases.

The DNA binding moiety comprises a polyamide segment that will bind selectively to the target CAG sequence. Polyamides designed by, for example, Dervan (U.S. Pat. Nos. 9,630,950 and 8,524,899) and others can selectively bind to selected DNA sequences. These polyamides sit in the minor groove of double helical DNA and form hydrogen bonding interactions with the Watson-Crick base pairs. Polyamides that selectively bind to particular DNA sequences can be designed by linking monoamide building blocks according to established chemical rules. One building block is provided for each DNA base pair, with each building block binding noncovalently and selectively to one of the DNA base pairs: A/T, T/A, G/C, and C/G. Following this guideline, trinucleotides binds to molecules with three amide units, i.e. tri-amides. In general, these polyamides can orient in either direction of a DNA sequence.

In principle, longer DNA sequences can be targeted with higher specificity and/or higher affinity by combining a larger number of monoamide building blocks into longer polyamide chains. Ideally, the binding affinity for a polyamide would simply be equal to the sum of each individual monoamide/DNA base pair interaction and/or heterocycle/DNA base pair interaction. In practice, however, due to the geometric mismatch between the fairly rigid polyamide and DNA structures, longer polyamide sequences do not bind to longer DNA sequences as tightly as would be expected from a simple additive contribution. The geometric mismatch between longer polyamide sequences and longer DNA sequences induces an unfavorable geometric strain that subtracts from the binding affinity that would be otherwise expected.

Disclosed herein are compounds that comprise a polyamide moiety that can bind to one or more copies of the CAG trinucleotide repeat sequence, and can modulate the expression of a target gene comprising a CAG trinucleotide repeat sequence. Treatment of a subject with these compounds will modulate expression of the defective target gene, and this can reduce the occurrence, severity, or frequency of symptoms associated with the disease. Certain compounds disclosed herein will provide higher binding affinity and selectivity than has been observed previously for this class of compounds.

It should be understood, however, that the detailed description and the specific examples, while indicating specific embodiments, are given by way of illustration only, since various changes and modifications within the spirit and scope of the instant disclosure will become apparent to those skilled in the art from this detailed description.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

The transcription modulator molecules described herein can be programmed to regulate the expression of a target gene containing a nucleotide repeat comprising CAG. In some embodiments, the modulator molecule comprises one or more nucleotide repeats comprising CAG.

The transcription modulator molecules contain DNA binding moieties that will selectively bind to one or more copies of the CAG trinucleotide repeat that is characteristic of the defective target gene (e.g., atxn1, atxn2, atxn3, cacna1a, atxn7, ttbk2, ppp2r2b, tbp, htt, jph3, ar, or atn1). The molecules and compounds disclosed herein provide higher binding affinity and selectivity than has been observed previously for this class of compounds and can be more effective in treating diseases associated with the defective target gene.

### Compounds—Transcription Modulator Molecules

In some embodiments, the transcription modulator molecule is a compound having a DNA-binding moiety capable of noncovalently binding to a nucleotide repeat sequence CAG. In some embodiments, the DNA binding moiety is a polyamide.

In some embodiments, the DNA binding moiety comprises one or more monomer subunits.

In some embodiments, the one or more subunits comprises —NH-Q-C(O)—, wherein Q is an optionally substituted C₆-C₁₀ arylene, optionally substituted 4 to 10-membered heterocyclene, optionally substituted 5 to 10-membered heteroarylene, or an optionally substituted alkylene.

In some embodiments, the DNA-binding moiety comprises a polyamide of one or more of the following subunits selected from:

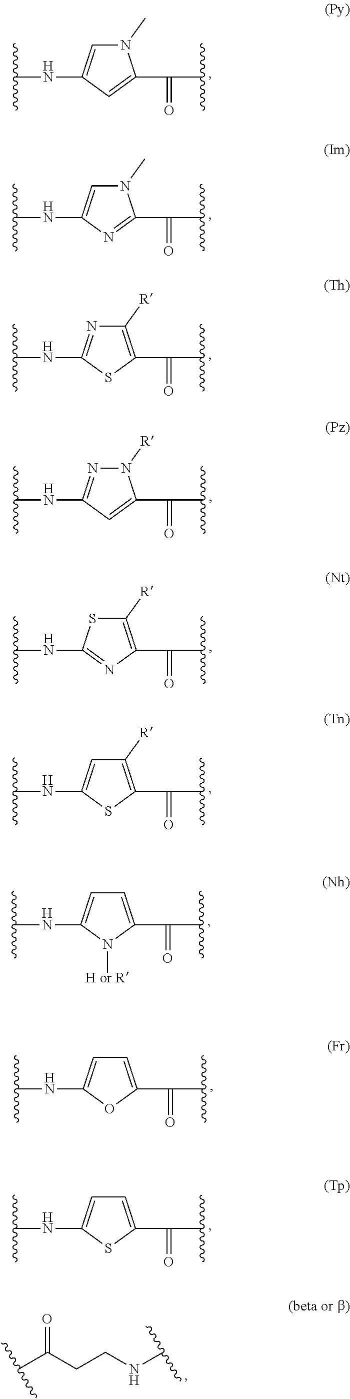

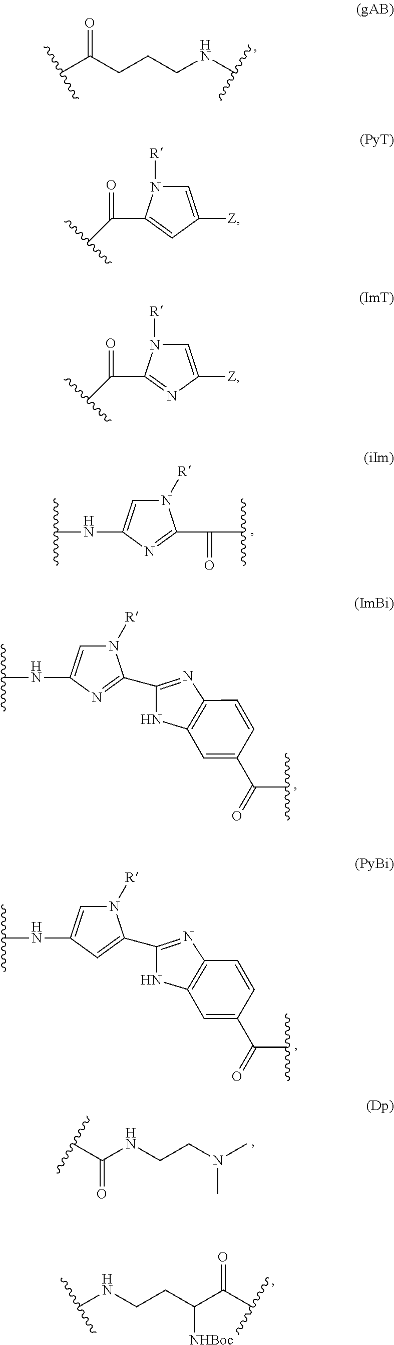

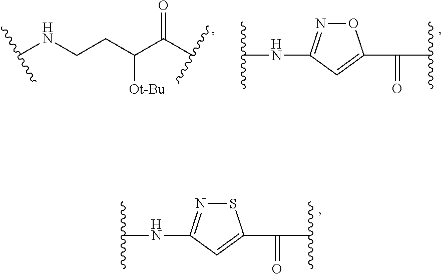 [SMILES: *NCCC(OC(C)(C)C)C(*)=O ; *Nc1cc(C(*)=O)on1 ; *Nc1cc(C(*)=O)sn1]

—NH-benzopyrazinylene-C(O)—, —NH-phenylene-C(O)—, —NH-pyridinylene-C(O)—, —NH-piperidinylene-C(O)—, —NH-pyrimidinylene-C(O)—, —NH-anthracenylene-C(O)—, —NH-quinolinylene-C(O)—, and

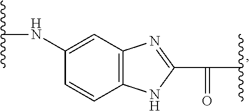

wherein each R′ is independently hydrogen, optionally substituted C₁-C₂₀ alkyl, optionally substituted C₁-C₂₀ heteroalkyl, optionally substituted C₁-C₂₀ haloalkyl, or optionally substituted C₁-C₂₀ alkylamino; and Z is H, NH₂, C₁-C₆ alkyl, C₁-C₆ haloalkyl, or C₁-C₆ alkyl-NH₂.

In some embodiments, the one or more subunits is independently selected from the group consisting of optionally substituted pyrrole carboxamide monomer, optionally substituted imidazole carboxamide monomer, optionally substituted δ-aminobutyric acid (gAB), and β-alanine (beta or β). In some embodiments, one or more of the polyamide backbone carbonyl groups (C═O), is replaced with an oxetane. In some embodiments, at least one of the polyamide backbone carbonyl groups is replaced with an oxetane.

In some embodiments, the polyamide comprises at least three aromatic carboxamide moieties selected to correspond to the nucleotide repeat sequence CAG and at least one aliphatic amino acid residue chosen from the group consisting of glycine, β-alanine, δ-aminobutyric acid, 2,4-diaminobutyric acid, and 5-aminovaleric acid. In some embodiments, the polyamide comprises one or more subunits selected from the group consisting of optionally substituted N-methylpyrrole carboxamide, optionally substituted N-methylimidazole carboxamide, β-alanine, and δ-aminobutyric acid. In some embodiments, the DNA-binding moiety comprises one δ-aminobutyric acid.

In an aspect, provided herein is a transcription modulator molecule having the structure of Formula (A), or a pharmaceutically acceptable salt thereof:

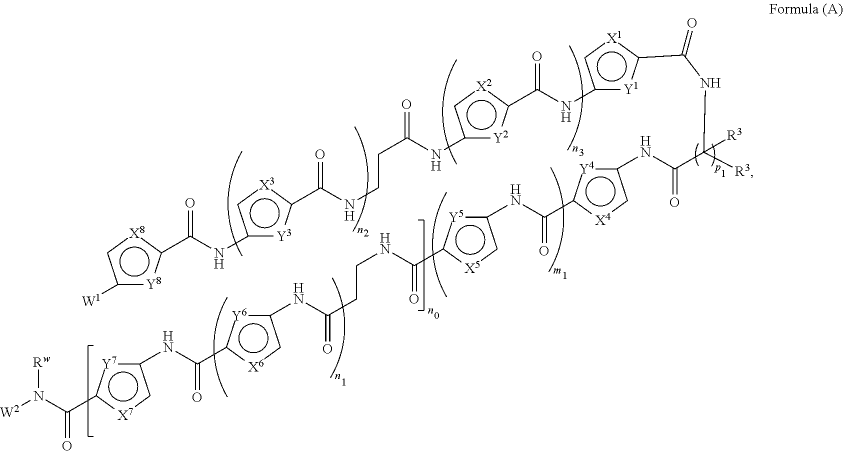

wherein:
each X¹, X², X³, X⁴, X⁵, X⁶, X⁷, and X⁸ is independently O or NR²;
each Y¹, Y², Y³, Y⁴, Y⁵, Y⁶, Y⁷, and Y⁸ is independently CH or N;
W¹ is hydrogen, halogen, optionally substituted C₁-C₁₀ alkyl, —NR^{1e}C(O)R^{1f}, —NR^{1e}C(O)NR^{1e}R^{1f}, —C(O)NR^{1e}R^{1f}, —OC(O)NR^{1e}R^{1f}, —NR^{1e}C(O)OR^{1f}, —N═C(N(PO(OR^{1e})₂—Z_{B}—(CH₂)ₚ₃—PO(OR^{1e})₂, or —Z_{B}—(CH₂)ₚ₃—O—PO(OR^{1e})₂, wherein
each R^{1e} is independently hydrogen, optionally substituted C₁-C₂₀ alkyl, optionally substituted C₂-C₂₀ alkenyl, optionally substituted C₂-C₂₀ alkynyl, optionally substituted C₁-C₂₀ heteroalkyl, optionally substituted C₂-C₂₀ heteroalkenyl, optionally substituted C₂-C₅₀ heteroalkynyl, or PEG₁₋₅₀;
each R^{1f} is independently hydrogen, optionally substituted C₁-C₂₀ alkyl, optionally substituted C₂-C₂₀ alkenyl, optionally substituted C₂-C₂₀ alkynyl, optionally substituted C₁-C₂₀ heteroalkyl, optionally substituted C₂-C₂₀ heteroalkenyl, optionally substituted C₂-C₂₀ heteroalkynyl, PEG₁₋₅₀, or AA₁₋₁₀;
or R^{1e} and R^{1f} together with the nitrogen to which they are attached form an optionally substituted 5 to 7-membered heterocycloalkyl, wherein
   each AA is independently a naturally occurring amino acid;
   Z_{B} is N or O; and
   p₃ is 1-10;
W² is hydrogen, optionally substituted C₁-C₂₀ alkyl, or optionally substituted C₁-C₂₀ heteroalkyl; or -L¹-Z—R⁴, wherein
   L¹ is alkylene or heteroalkylene;
   Z is absent, —C(O)—, or —C(═NH)—; and
   R⁴ is C₁-C₆ alkyl, —OR^{4b}, or —NR^{4a}R^{4b}; wherein
      R^{4a} is hydrogen, optionally substituted C₁-C₂₀ alkyl, or optionally substituted C₁-C₂₀ heteroalkyl;
      R^{4b} is optionally substituted C₁-C₂₀ alkyl, optionally substituted C₂-C₂₀ alkenyl, optionally substituted C₂-C₂₀ alkynyl, optionally substituted C₁-C₂₀ aminoalkyl, optionally substituted C₁-C₂₀ haloalkyl, optionally substituted C₁-C₂₀ heteroalkyl, optionally substituted C₁-C₂₀ hydroxyalkyl, optionally substituted C₃-C₅ cycloalkyl. optionally substituted 4 to 8-membered heterocycloalkyl, optionally substituted phenyl, or optionally substituted 5 to 10-membered heteroaryl; or
      R^{4a} and R^{4b} together with the nitrogen to which they are attached form an optionally substituted 4 to 8-membered heterocycloalkyl which is partially or fully unsaturated;
R^{W} is hydrogen or C₁-C₂₀ alkyl;
or W² and R^{W} together with the nitrogen to which they are attached form an optionally substituted 4 to 8-membered heterocycloalkyl which is partially or fully unsaturated;
each R² is independently hydrogen, optionally substituted C₁-C₅₀ alkyl, optionally substituted C₂-C₅₀ alkenyl, optionally substituted C₂-C₅₀ alkynyl, optionally substituted C₁-C₅₀ heteroalkyl, optionally substituted C₂-C₅₀ heteroalkenyl, optionally substituted C₂-C₅₀ heteroalkynyl, optionally substituted C₁-C₅₀ hydroxyalkyl, optionally substituted C₁-C₅₀ aminoalkyl, optionally substituted C₁-C₅₀ haloalkyl, optionally substituted C₃-C₈ cycloalkyl, optionally substituted 3 to 8-membered heterocycloalkyl, or optionally substituted PEG₁₋₅₀;
each R³ is independently hydrogen, deuterium, halogen, acetyl, amino, amido, optionally substituted C₁-C₂₀ alkyl, optionally substituted C₁-C₂₀ haloalkyl, optionally substituted C₁-C₂₀ alkylamino, or optionally substituted C₁-C₂₀ hydroxyalkyl; or
two R³ together with the atom(s) to which they are attached form a C₃-C₆ cycloalkyl or 3 to 6-membered heterocycloalkyl;
p₁ is 3 or 4;
n₁ and n₂ are each independently 0 or 1;
n₃ is 1 or 2;
m₁ is 0, 1, 2, or 3; and
n₀ is 0 or 1, wherein no and m₁ are both not 0.

In some embodiments of Formula (A), n₀ is 1. In some embodiments, n₀ is 0.

In some embodiments of Formula (A), n₂ is 2. In some embodiments, n₂ is 1. In some embodiments, n₂ is 0.

In some embodiments of Formula (A), n₃ is 1. In some embodiments, n₃ is 0.

In another aspect, provided herein is a transcription modulator molecule having the structure of Formula (A-1), or a pharmaceutically acceptable salt thereof:

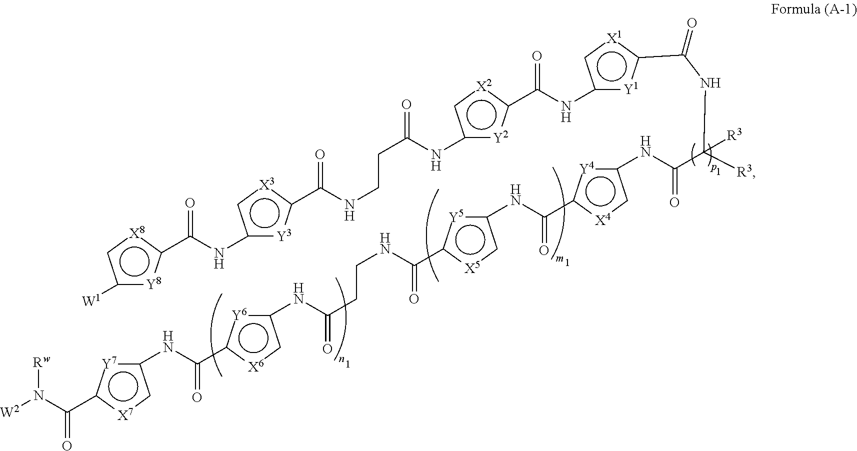

wherein:
each X¹, X², X³, X⁴, X⁵, X⁶, X⁷, and X⁸ is independently O or NR²;
each Y¹, Y², Y³, Y⁴, Y⁵, Y⁶, Y⁷, and Y⁸ is independently CH or N;
W¹ is hydrogen, halogen, optionally substituted C₁-C₁₀ alkyl, —NR^{1e}C(O)R^{1f}, —NR^{1e}C(O)NR^{1e}R^{1f}, —C(O)NR^{1e}R^{1f}—OC(O)NR^{1e}R^{1f}, —NR^{1e}C(O)OR^{1f}, —N═C(N(R^{1e})₂)₂, AA₁₋₁₀, —Z_{B}—PO(OR^{1e})₂, —Z_{B}—(CH₂)ₚ₃—PO(OR^{1e})₂, or —Z_{B}—(CH₂)ₚ₃—O—PO(OR^{1e})₂, wherein
each R^{1e} is independently hydrogen, optionally substituted C₁-C₂₀ alkyl, optionally substituted C₂-C₂₀ alkenyl, optionally substituted C₂-C₂₀ alkynyl, optionally substituted C₁-C₂₀ heteroalkyl, optionally substituted C₂-C₂₀ heteroalkenyl, optionally substituted C₂-C₅₀ heteroalkynyl, or PEG₁₋₅₀;
each R^{1f} is independently hydrogen, optionally substituted C₁-C₂₀ alkyl, optionally substituted C₂-C₂₀ alkenyl, optionally substituted C₂-C₂₀ alkynyl, optionally substituted C₁-C₂₀ heteroalkyl, optionally substituted C₂-C₂₀ heteroalkenyl, optionally substituted C₂-C₂₀ heteroalkynyl, PEG₁₋₅₀, or (AA)₁₋₁₀;
or R^{1e} and R^{1f} together with the nitrogen to which they are attached form an optionally substituted 5 to 8-membered heterocycloalkyl, wherein
   each AA is independently a naturally occurring amino acid;
   Z_{B} is N or O; and
   p₃ is 1-10;
W² is hydrogen, optionally substituted C₁-C₂₀ alkyl, or optionally substituted C₁-C₂₀ heteroalkyl, or -L¹-Z—R⁴; wherein
   L¹ is alkylene or heteroalkylene;
   Z is absent, —C(O)—, or —C(═NH)—; and
   R⁴ is —C₁-C₆ alkyl, —OR^{4b}, or —NR^{4a}R^{4b}; wherein
      R^{4a} is hydrogen, optionally substituted C₁-C₂₀ alkyl, or optionally substituted C₁-C₂₀ heteroalkyl;
      R^{4b} is optionally substituted C₁-C₂₀ alkyl, optionally substituted C₂-C₂₀ alkenyl, optionally substituted C₂-C₂₀ alkynyl, optionally substituted C₁-C₂₀ aminoalkyl, optionally substituted C₁-C₂₀ haloalkyl, optionally substituted C₁-C₂₀ heteroalkyl, optionally substituted C₁-C₂₀ hydroxyalkyl, optionally substituted C₃-C₇ cycloalkyl. optionally substituted 4 to 8-membered heterocycloalkyl, optionally substituted phenyl, or optionally substituted 5 to 10-membered heteroaryl; or
      R^{4a} and R^{4b} together with the nitrogen to which they are attached form an optionally substituted 4 to 8-membered heterocycloalkyl which is partially or fully unsaturated;
R^{W} is hydrogen or C₁-C₂₀ alkyl;
or W² and R^{W} together with the nitrogen to which they are attached form an optionally substituted 4 to 10-membered heterocycloalkyl which is partially or fully unsaturated;
each R² is independently hydrogen, optionally substituted C₁-C₅₀ alkyl, optionally substituted C₂-C₅₀ alkenyl, optionally substituted C₂-C₅₀ alkynyl, optionally substituted C₁-C₅₀ heteroalkyl, optionally substituted C₂-C₅₀ heteroalkenyl, optionally substituted C₂-C₅₀ heteroalkynyl, optionally substituted C₁-C₅₀ aminoalkyl, optionally substituted C₁-C₅₀ haloalkyl, optionally substituted C₁-C₅₀ hydroxyalkyl, optionally substituted C₃-C₈ cycloalkyl, optionally substituted 3 to 8-membered heterocycloalkyl ring, or optionally substituted PEG₁₋₅₀;
each R³ is independently hydrogen, deuterium, halogen, acetyl, amino, amido, optionally substituted C₁-C₂₀ alkyl, optionally substituted C₁-C₂₀ haloalkyl, optionally substituted C₁-C₂₀ alkylamino, or optionally substituted C₁-C₂₀ hydroxyalkyl; or
two R³ together with the atom(s) to which they are attached form a C₃-C₆ cycloalkyl or 4 to 6-membered heterocycloalkyl;
p₁ is 3 or 4; and
m₁ and n₁ are each independently 0 or 1.

In some embodiments of Formula (A) or (A-1), p₁ is 3. In some embodiments, p₁ is 4.

In some embodiments of Formula (A) or (A-1), each X¹, X², X³, X⁴, X⁵, X⁶, X⁷, and X⁸ is independently NR². In some embodiments, each X¹, X², X³, X⁴, X⁵, X⁶, X⁷, and X⁸ is independently O.

In some embodiments of Formula (A) or (A-1), W¹ is hydrogen, halogen, optionally substituted C₁-C₁₀ alkyl, —NR^{1e}C(O)R^{1f}, —NR^{1e}C(O)NR^{1e}R^{1f}, —C(O)NR^{1e}R^{1f}, —OC(O)NR^{1e}R^{1f}, —NR^{1e}C(O)OR^{1f}, or AA₁₋₃.

In some embodiments, W¹ is hydrogen or optionally substituted C₁-C₁₀ alkyl. In some embodiments, W¹ is —NR^{1e}C(O)R^{1f}, —NR^{1e}C(O)NR^{1e}R^{1f}, —C(O)NR^{1e}R^{1f}, —OC(O)NR^{1e}R^{1f}, or —NR^{1e}C(O)OR^{1f}. In some embodiments, W¹ is AA₁₋₁₀. In some embodiments, W¹ is AA₁₋₄. In some embodiments, W¹ is AA₁₋₃.

In some embodiments, W¹ is —Z_{B}—PO(OR^{1e})₂, —Z_{B}—(CH₂)ₚ₃—PO(OR^{1e})₂, or —Z_{B}—(CH₂)ₚ₃—O—PO₂(OR^{1e})₂, wherein Z_{B} is O or N, and p₃ is 1-10.

In some embodiments of Formula (A) or (A-1), W¹ is (azaneylidene)methanediamine or (azaneylidene)-N,N,N′,N′-tetramethylmethanediamine.

In some embodiments of Formula (A) or (A-1), W¹ is guanidinyl. In some embodiments, W¹ is —N═C(N(R^{1e})₂)₂, wherein each R^{1e} is independently hydrogen, optionally substituted C₁-C₂₀ alkyl, optionally substituted C₂-C₂₀ alkenyl, optionally substituted C₂-C₂₀ alkynyl, optionally substituted C₁-C₂₀ heteroalkyl, optionally substituted C₂-C₂₀ heteroalkenyl, optionally substituted C₂-C₅₀ heteroalkynyl, or PEG₁₋₅₀.

In some embodiments of Formula (A) or (A-1), W¹ is

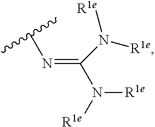

wherein each R^{1e} is independently hydrogen or an optionally substituted C₁-C₂₀ alkyl.

In some embodiments of Formula (A) or (A-1), W¹ is

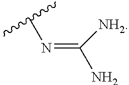 [SMILES: *N=C(N)N]

In some embodiments, W¹ is

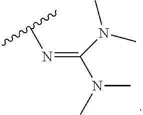 [SMILES: *N=C(N(C)C)N(C)C]

In some embodiments of Formula (A) or (A-1), W¹ is hydrogen or —N═C(N(R^{1c})₂)₂, wherein each R^{1c} is independently hydrogen or C₁-C₃ alkyl. In some embodiments, W¹ is hydrogen or —N═C(NR^{1c})₂)₂, wherein each R^{1c} is independently hydrogen or methyl.

In some embodiments of Formula (A) or (A-1), W¹ is hydrogen.

In some embodiments of Formula (A) or (A-1), each R^{1c} is independently an optionally substituted C₁-C₂₀ alkyl, optionally substituted C₂-C₂₀ alkenyl, optionally substituted C₂-C₂₀ alkynyl, optionally substituted C₁-C₂₀ heteroalkyl, optionally substituted C₂-C₂₀ heteroalkenyl, optionally substituted C₂-C₅₀ heteroalkynyl, or PEG₁₋₅₀. In some embodiments, each R^{1e} is independently an optionally substituted C₁-C₂₀ alkyl, optionally substituted C₂-C₂₀ alkenyl, or optionally substituted C₂-C₂₀ alkynyl. In some embodiments, each R^{1e} is independently an optionally substituted C₁-C₂₀ heteroalkyl, optionally substituted C₂-C₂₀ heteroalkenyl, optionally substituted C₂-C₅₀ heteroalkynyl, or PEG₁₋₅₀. In some embodiments, each R^{1e} is independently PEG₁₋₅₀. In some embodiments, each R^{1e} is independently hydrogen.

In some embodiments of Formula (A) or (A-1), each R^{1e} is independently hydrogen, optionally substituted C₁-C₂₀ alkyl, optionally substituted C₂-C₂₀ alkenyl, optionally substituted C₂-C₂₀ alkynyl, optionally substituted C₁-C₂₀ heteroalkyl, optionally substituted C₂-C₂₀ heteroalkenyl, optionally substituted C₂-C₂₀ heteroalkynyl, PEG₁₋₅₀, or AA₁₋₁₀. In some embodiments, each R^{1f} is independently an optionally substituted C₁-C₂₀ alkyl, optionally substituted C₂-C₂₀ alkenyl, optionally substituted C₂-C₂₀ alkynyl, optionally substituted C₁₋₂₀ heteroalkyl, optionally substituted C₂-C₂₀ heteroalkenyl, optionally substituted C₂-C₅₀ heteroalkynyl, or PEG₁₋₅₀. In some embodiments, each R^{1f} is independently an optionally substituted C₁-C₂₀ alkyl, optionally substituted C₂-C₂₀ alkenyl, or optionally substituted C₂-C₂₀ alkynyl. In some embodiments, each R^{1f} is independently an optionally substituted C₁-C₂₀ heteroalkyl, optionally substituted C₂-C₂₀ heteroalkenyl, optionally substituted C₂-C₅₀ heteroalkynyl, or PEG₁₋₅₀. In some embodiments, each R^{1f} is independently PEG₁₋₅₀. In some embodiments, each R^{1f} is independently hydrogen.

In some embodiments of Formula (A) or (A-1), R^{1e} and R^{1f} together with the nitrogen atom to which they are attached form an optionally substituted 4 to 8-membered heterocycloalkyl. In some embodiments, R^{1e} and R^{1f} together with the nitrogen atom to which they are attached form an optionally substituted 5 to 7-membered heterocycloalkyl. In some embodiments, R^{1e} and R^{1f} together with the nitrogen atom to which they are attached form an optionally substituted 5-membered heterocycloalkyl. In some embodiments, R^{1e} and R^{1f} together with the nitrogen atom to which they are attached form an optionally substituted 6-membered heterocycloalkyl. In some embodiments, R^{1e} and R^{1f} together with the nitrogen atom to which they are attached form an optionally substituted 7-membered heterocycloalkyl.

In some embodiments of Formula (A) or (A-1), each AA is independently a naturally occurring amino acid. In some embodiments, each AA is independently selected from lysine, arginine, serine, threonine, or cysteine. In some embodiments, each AA is independently selected from lysine or arginine. In some embodiments, each AA is independently selected from lysine. In some embodiments, each AA is independently selected from arginine.

In some embodiments of Formula (A) or (A-1), each R² is independently an optionally substituted C₁-C₅₀ alkyl, optionally substituted C₂-C₅₀ alkenyl, optionally substituted C₂-C₅₀ alkynyl, optionally substituted C₂-C₅₀ heteroalkyl, optionally substituted C₂-C₅₀ heteroalkenyl, optionally substituted C₂-C₅₀ heteroalkynyl, optionally substituted C₂-C₅₀ aminoalkyl, optionally substituted C₁-C₅₀ hydroxyalkyl, optionally substituted C₂-C₅₀ haloalkyl, optionally substituted C₃-C₈ cycloalkyl, optionally substituted 3 to 8-membered heterocycloalkyl, or optionally substituted PEG₁₅₀. In some embodiments, each R² is independently an optionally substituted C₁-C₅₀ alkyl, optionally substituted C₁-C₅₀ aminoalkyl, optionally substituted C₁-C₅₀ hydroxyalkyl, or optionally substituted PEG₁₋₅₀. In some embodiments, each R² is independently an optionally substituted C₁-C₃₀ alkyl, optionally substituted C₂-C₅₀ aminoalkyl, optionally substituted C₁-C₃₀ hydroxyalkyl, or optionally substituted PEG₁₋₃₀. In some embodiments, each R² is independently an optionally substituted C₁-C₂₀ alkyl, optionally substituted C₁-C₂₀ aminoalkyl, optionally substituted C₁-C₂₀ hydroxyalkyl, or optionally substituted PEG₁₋₂₀. In some embodiments, each R² is independently an optionally substituted C₁-C₁₀ alkyl, optionally substituted C₁-C₁₀ aminoalkyl, optionally substituted C₁-C₁₀ hydroxyalkyl, or optionally substituted PEG₁₋₁₀. In some embodiments, each R² is optionally substituted with one or more amino, amido, azido, cyano, ester, oxo (═O), urea, PEG, optionally substituted aryl, or optionally substituted 5 to 10-membered heteroaryl.

In some embodiments of Formula (A) or (A-1), each R² is independently hydrogen or an optionally substituted C₁-C₅₀ alkyl, which is optionally substituted with one or more amino, amido, azido, cyano, ester, oxo (═O), urea, optionally substituted aryl, or optionally substituted 5 to 10-membered heteroaryl. In some embodiments, each R² is independently an optionally substituted C₁-C₃₀ alkyl. In some embodiments, each R² is independently an optionally substituted C₁-C₂₀ alkyl. In some embodiments, each R² is independently an optionally substituted C₁-C₁₀ alkyl. In some embodiments, each R² is independently methyl, ethyl, isopropyl, isobutyl, sec-butyl, or tert-butyl. In some embodiments, each R² is independently hydrogen or methyl. In some embodiments, each R² is ethyl. In some embodiments, each R² is isopropyl. In some embodiments, each R² is methyl. In some embodiments, each R² is hydrogen.

In some embodiments of Formula (A) or (A-1), each R³ is independently hydrogen, halogen, amino, amido, optionally substituted C₁-C₂₀ alkyl, optionally substituted C₁-C₂₀ haloalkyl, optionally substituted C₁-C₂₀ alkylamino, or optionally substituted C₁-C₂₀ hydroxyalkyl. In some embodiments, each R³ is independently hydrogen, amino, amido, or optionally substituted C₁-C₂₀ alkylamino. In some embodiments, each R³ is independently hydrogen, amino, or amido. In some embodiments, each R³ is independently amino. In some embodiments, each R³ is independently amido. In some embodiments, each R³ is hydrogen.

In some embodiments of Formula (A) or (A-1), two R³ together with the atom(s) to which they are attached form a C₃-C₆ cycloalkyl or a 3 to 6-membered heterocycloalkyl. In some embodiments, two R³ together with the atom(s) to which they are attached form a C₃-C₆ cycloalkyl. In some embodiments, two R³ together with the atom(s) to which they are attached form a 4 to 6-membered heterocycloalkyl. In some embodiments, two R³ together with the atom(s) to which they are attached form a 4-membered heterocycloalkyl. In some embodiments, two R³ together with the atom(s) to which they are attached form a 5-membered heterocycloalkyl. In some embodiments, two R³ together with the atom(s) to which they are attached form a 6-membered heterocycloalkyl. In some embodiments, two R³ together with the atom(s) to which they are attached form a cyclopropyl, cyclobutyl, or cyclopentyl. In some embodiments, two R³ together with the atom(s) to which they are attached form a cyclopropyl. In some embodiments, two R³ together with the atom(s) to which they are attached form a cyclobutyl. In some embodiments, two R³ together with the atom(s) to which they are attached form a cyclopentyl.

In some embodiments of Formula (A) or (A-1), W² is -L¹-Z—R⁴; wherein L¹ is alkylene or heteroalkylene; Z is absent, —C(O)—, or —C(═NH)—; and R⁴ is —OR^{4b} or —NR^{4a}R^{4b}. In some embodiments, W² is -L¹-Z—R⁴; wherein L¹ is C₁-C₂₀ alkylene or C₂-C₂₀ heteroalkylene; Z is absent, —C(O)—, or —C(═NH)—; and R⁴ is —OR^{4b} or —NR^{4a}R^{4b}. In some embodiments, W² is -L¹-Z—R⁴; wherein L¹ is C₁-C₁₀ alkylene or C₂-C₁₀ heteroalkylene; Z is absent, —C(O)—, or —C(═NH)—; and R⁴ is —OR^{4b} or —NR^{4a}R^{4b}. In some embodiments, W² is -L¹-Z—R⁴; wherein L¹ is C₁-C₈ alkylene or C₂-C₈ heteroalkylene; Z is absent, —C(O)—, or —C(═NH)—; and R⁴ is —OR^{4b} or —NR^{4a}R^{4b}. In some embodiments, W² is -L¹-Z—R⁴; wherein L¹ is C₁-C₆ alkylene or C₂-C₆ heteroalkylene; Z is absent, —C(O)—, or —C(═NH)—; and R^{4a} is —OR^{4b} or —NR^{4a}R^{4b}.

In some embodiments of Formula (A) or (A-1), W² is -L¹-Z—R⁴; wherein L¹ is C₁-C₂₀ alkylene; Z is absent, —C(O)—, or —C(═NH)—; and R⁴ is —NR^{4a}R^{4b}. In some embodiments, W² is -L¹-Z—R⁴; wherein L¹ is C₁-C₁₀ alkylene; Z is absent, —C(O)—, or —C(═NH)—; and R⁴ is —NR^{4a}R^{4b}. In some embodiments, W² is -L¹-Z—R⁴; wherein L¹ is C₁-C₈ alkylene; Z is absent, —C(O)—, or —C(═NH)—; and R⁴ is —NR^{4a}R^{4b}. In some embodiments, W² is -L¹-Z—R⁴; wherein L¹ is C₁-C₆ alkylene; Z is absent, —C(O)—, or —C(═NH)—; and R⁴ is —NR^{4a}R^{4b}.

In some embodiments of Formula (A) or (A-1), W² is -L¹-Z—R⁴; wherein L¹ is C₁-C₂₀ alkylene or C₂-C₂₀ heteroalkylene; Z is absent or —C(O)—; and R⁴ is —OR^{4b} or —NR^{4a}R^{4b}. In some embodiments, W² is -L¹-Z—R⁴; wherein L¹ is C₁-C₁₀ alkylene or C₂-C₁₀ heteroalkylene; Z is absent or —C(O)—; and R⁴ is —OR^{4b} or —NR^{4a}R^{4b}. In some embodiments, W² is -L¹-Z—R⁴; wherein L¹ is C₁-C₈ alkylene or C₂-C₅ heteroalkylene; Z is absent or —C(O)—; and R⁴ is —OR^{4b} or —NR^{4a}R^{4b}. In some embodiments, W² is -L¹-Z—R⁴; wherein L¹ is C₁-C₆ alkylene or C₂-C₆ heteroalkylene; Z is absent or —C(O)—; and R⁴ is —OR^{4b} or —NR^{4a}R^{4b}.

In some embodiments of Formula (A) or (A-1), W² is -L¹-Z—R⁴; wherein L¹ is C₁-C₂₀ alkylene; Z is —C(O)—; and R⁴ is —NR^{4a}R^{4b}. In some embodiments, W² is -L¹-Z—R⁴; wherein L¹ is C₁-C₁₀ alkylene; Z is —C(O)—; and R⁴ is —NR^{4a}R^{4b}. In some embodiments, W² is -L¹-Z—R⁴; wherein L¹ is C₁-C₈ alkylene; Z is —C(O)—; and R⁴ is —NR^{4a}R^{4b}. In some embodiments, W² is -L¹-Z—R⁴; wherein L¹ is C₁-C₆ alkylene; Z is —C(O)—; and R⁴ is —NR^{4a}R^{4b}.

In some embodiments of Formula (A) or (A-1), W² is -L¹-Z—R⁴; wherein L¹ is C₁-C₂₀ alkylene; Z is absent; and R⁴ is —NR^{4a}R^{4b}. In some embodiments, W² is -L¹-Z—R⁴; wherein L¹ is C₁-C₁₀ alkylene; Z is absent; and R⁴ is —NR^{4a}R^{4b}. In some embodiments, W² is -L¹-Z—R⁴; wherein L¹ is C₁-C₈ alkylene; Z is absent; and R⁴ is —NR^{4a}R^{4b}. In some embodiments, W² is -L¹-Z—R⁴; wherein L¹ is C₁-C₆ alkylene; Z is absent; and R⁴ is —NR^{4a}R^{4b}.

In some embodiments of Formula (A) or (A-1), W² is -L¹-Z—R⁴; wherein L¹ is C₁-C₂₀ alkylene or C₁-C₂₀ heteroalkyl; Z is absent or —C(O)—; and R⁴ is C₁-C₆ alkyl. In some embodiments, W² is -L¹-Z—R⁴; wherein L¹ is C₁-C₂₀ alkylene; Z is absent or —C(O)—; and R⁴ is C₁-C₆ alkyl. In some embodiments, W² is -L¹-Z—R⁴; wherein L¹ is C₁-C₂₀ alkylene; Z is —C(O)—; and R⁴ is C₁-C₆ alkyl. In some embodiments, W² is -L¹-Z—R⁴; wherein L¹ is C₁-C₂₀ alkylene; Z is absent; and R⁴ is C₁-C₆ alkyl.

In another aspect, provided herein is a transcription modulator molecule having a structure of Formula (I), or a pharmaceutically acceptable salt thereof:

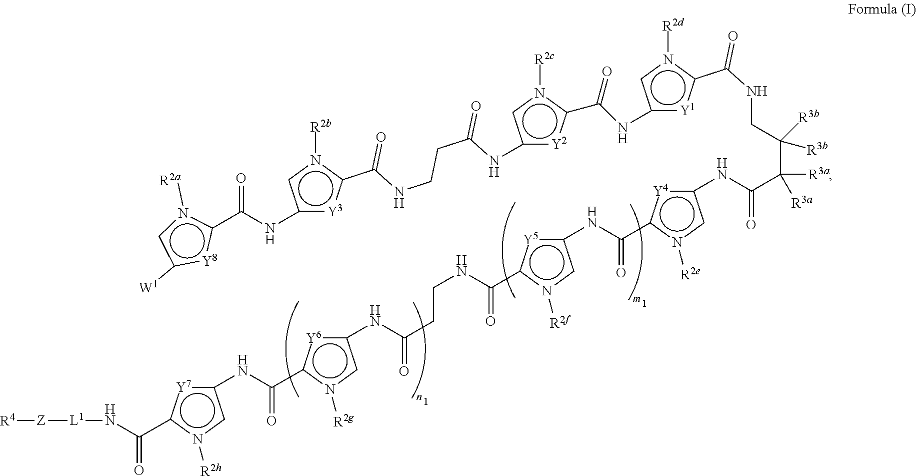

wherein:
W¹ is hydrogen or —N═C(N(R^{1e})₂)₂, wherein each R^{1e} is independently hydrogen or C₁-C₃ alkyl;
each Y¹, Y², Y³, Y⁴, Y⁵, Y⁶, Y⁷, and Y⁸ is independently N or CH;
L¹ is C₁-C₂₀ alkylene or C₁-C₂₀ heteroalkylene;
Z is absent, —C(O)—, or —C(═NH)—;
R⁴ is C₁-C₆ alkyl, —OR^{4b}, or —NR^{4a}R^{4b}; wherein
   R^{4a} is hydrogen, optionally substituted C₁-C₂₀ alkyl, or optionally substituted C₁-C₂₀ heteroalkyl;
   R^{4b} is optionally substituted C₁-C₂₀ alkyl, optionally substituted C₁-C₂₀ alkenyl, optionally substituted C₁-C₂₀ alkynyl, optionally substituted C₁-C₂₀ aminoalkyl, optionally substituted C₁-C₂₀ haloalkyl, optionally substituted C₁-C₂₀ heteroalkyl, optionally substituted C₁-C₂₀ hydroxyalkyl, optionally substituted C₃-C₈ cycloalkyl, optionally substituted 4 to 8-membered heterocycloalkyl, optionally substituted phenyl, or optionally substituted 5 to 10-membered heteroaryl; or
   R^{4a} and R^{4b} together with the nitrogen to which they are attached form an optionally substituted 4 to 8-membered heterocycloalkyl which is partially or fully unsaturated;
each R^{2a}, R^{2b}, R^{2c}, R^{2d}, R^{2e}, R^{2f}, R^{2g}, and R^{2h} is independently hydrogen, optionally substituted C₁-C₅₀ alkyl, optionally substituted C₂-C₅₀ alkenyl, optionally substituted C₂-C₅₀ alkynyl, optionally substituted C₁-C₅₀ heteroalkyl, optionally substituted C₂-C₅₀ heteroalkenyl, optionally substituted C₂-C₅₀ heteroalkynyl, optionally substituted C₁-C₅₀ haloalkyl, optionally substituted C₃-C₈ cycloalkyl, optionally substituted 3 to 8-membered heterocycloalkyl, or optionally substituted PEG₁₋₅₀; each of which is optionally substituted with one or more R¹⁰;
each R^{3a} and R^{3b} is independently hydrogen, halogen, —NR^{11a}R^{11b}, or —NHC(O)R¹², wherein
   R^{11a} and R^{11b} are each independently hydrogen, alkyl, or PEG;
   R¹² is alkyl, PEG, cycloalkyl, heterocycloalkyl, or phenyl;
or two R^{3a} or two R^{3b} together with the carbon atom to which they are attached form a C₃-C₆ cycloalkyl or 4 to 6-membered heterocycloalkyl;
each R¹⁰ is independently —CN, —OH, —OR^{10a}, —N₃, —NR^{10a}R^{10b}, —CO(O)R^{10c}, —C(O)OR^{10c}, —C(O)NR^{10a}R^{10b}, —NHC(O)R^{10c}, —NHC(O)OR^{10c}, —OC(O)NR^{10a}R^{10b}, or optionally substituted 5 to 10-membered heteroaryl; wherein
   R^{10a} and R^{10b} are each independently hydrogen, alkyl, or PEG;
   R^{10c} is alkyl, PEG, cycloalkyl, heterocycloalkyl, or phenyl; and
n₁ and m₁ are each independently 0 or 1.

In another aspect, provided herein is a transcription modulator molecule having a structure of Formula (I), or a pharmaceutically acceptable salt thereof:

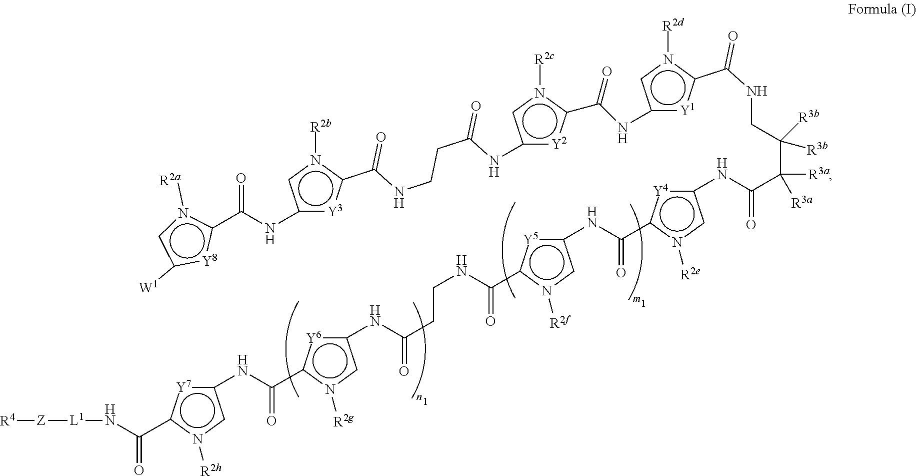

wherein:
W¹ is hydrogen;
each Y⁵ is independently CH or N;
L¹ is C₁-C₂₀ alkylene;
Z is absent or —C(O)—;
R⁴ is —NR^{4a}R^{4b}; wherein
   R^{4a} is hydrogen, optionally substituted C₁-C₂₀ alkyl, or optionally substituted C₁-C₂₀ heteroalkyl;
   R^{4b} is optionally substituted C₁-C₂₀ alkyl, optionally substituted C₁-C₂₀ aminoalkyl, optionally substituted C₁-C₂₆ haloalkyl, optionally substituted C₁-C₂₀ heteroalkyl, optionally substituted C₁-C₂₀ hydroxyalkyl, optionally substituted C₃-C₈ cycloalkyl, optionally substituted 4 to 8-membered heterocycloalkyl, optionally substituted phenyl, or optionally substituted 5 to 10-membered heteroaryl; or
   R^{4a} and R^{4b} together with the nitrogen to which they are attached form an optionally substituted 4 to 8-membered heterocycloalkyl;
each R^{2a}, R^{2b}, R^{2c}, R^{2d}, R^{2e}, R^{2f}, R^{2g}, and R^{2h} is independently hydrogen, optionally substituted C₁-C₁₀ alkyl, optionally substituted C₁-C₁₀ heteroalkyl, optionally substituted C₁-C₁₀ haloalkyl, or optionally substituted PEG₁₋₁₀; each of which is optionally substituted with one or more R¹⁰;
each R^{3a} and R^{3b} is independently hydrogen, —NR^{11a}R^{11b}, or —NHC(O)R¹², wherein
   R^{11a} and R^{11b} are each independently hydrogen, alkyl, or PEG;
   R¹² is alkyl, PEG, cycloalkyl, heterocycloalkyl, or phenyl;
or two R^{3a} or two R^{3b} together with the carbon atom to which they are attached form a C₃-C₆ cycloalkyl or 4 to 6-membered heterocycloalkyl;
each R¹⁰ is independently —CN, —OH, —OR^{10a}, —N₃, —NR^{10a}R^{10b}, —CO(O)R^{10c}, —C(O)OR^{10c}, —C(O)NR^{10a}R^{10b}, —NHC(O)R^{10c}, —NHC(O)OR^{10c}, —OC(O)NR^{10a}R^{10b}, or optionally substituted 5 to 10-membered heteroaryl; wherein
   R^{10a} and R^{10b} are each independently hydrogen, alkyl, or PEG;
   R^{10c} is alkyl, PEG, cycloalkyl, heterocycloalkyl, or phenyl; and
n₁ and m₁ are each independently 0 or 1.

In some embodiments, the molecule of Formula (I) has the structure of Formula (Ia), or a pharmaceutically acceptable salt thereof:

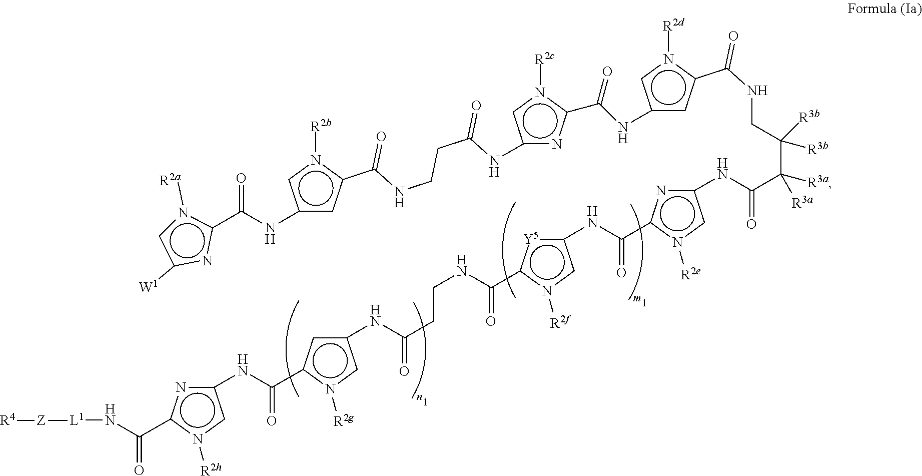

wherein:
W¹ is hydrogen or —N═C(N(R^{1e})₂)₂, wherein each R^{1e} is independently hydrogen or C₁-C₃ alkyl;
each Y⁵ is CH or N;
L¹ is C₁-C₂₀ alkylene or C₂-C₂₀ heteroalkylene;
Z is absent, —C(O)—, or —C(═NH)—;
R⁴ is C₁-C₆ alkyl, —OR^{4b} or —NR^{4a}R^{4b}; wherein
   R^{4a} is hydrogen, optionally substituted C₁-C₂₀ alkyl, or optionally substituted C₁-C₂₀ heteroalkyl;
   R^{4b} is optionally substituted C₁-C₂₀ alkyl, optionally substituted C₁-C₂₀ alkenyl, optionally substituted C₁-C₂₀ alkynyl, optionally substituted C₁-C₂₀ aminoalkyl, optionally substituted C₁-C₂₀ haloalkyl, optionally substituted C₁-C₂₀ heteroalkyl. optionally substituted C₁-C₂₀ hydroxyalkyl, optionally substituted C₃-C₇ cycloalkyl, optionally substituted 4 to 8-membered heterocycloalkyl, optionally substituted phenyl, or optionally substituted 5 to 10-membered heteroaryl; or
   R^{4a} and R^{4b} together with the nitrogen to which they are attached form an optionally substituted 4 to 8-membered heterocycloalkyl which is partially or fully unsaturated:
each R^{2a}, R^{2b}, R^{2c}, R^{2d}, R^{2e}, R^{2f}, R^{2g}, and R^{2h} is independently hydrogen, optionally substituted C₁-C₅₀ alkyl, optionally substituted C₁-C₅₀ alkenyl, optionally substituted C₁-C₅₀ alkynyl, optionally substituted C₁-C₅₀ heteroalkyl, optionally substituted C₁-C₅₀ heteroalkenyl, optionally substituted C₂-C₅₀ heteroalkynyl, optionally substituted C₁-C₅₀ haloalkyl, optionally substituted C₃-C₈ cycloalkyl, optionally substituted 3 to 8-membered heterocycloalkyl, or optionally substituted PEG₁₋₅₀; each of which is optionally substituted with one or more R¹⁰;
each R^{3a} and R^{3b} is independently hydrogen, halogen, —NR^{11a}R^{11b}, or —NHC(O)R¹², wherein
   R^{11a} and R^{11b} are each independently hydrogen, alkyl, or PEG;
   R¹² is alkyl, PEG, cycloalkyl, heterocycloalkyl, or phenyl:
or two R^{3a} or two R^{3b} together with the carbon atom to which they are attached form a C₃-C₆ cycloalkyl or 4 to 6-membered heterocycloalkyl;
each R¹⁰ is independently —CN, —OH, —OR^{10a}, —N₃, —NR^{10a}R^{10b}, —CO(O)R^{10c}, —C(O)OR^{10c}, —C(O)NR^{10a}R^{10b}, —NHC(O)R^{10c}, —NHC(O)OR^{10c}, —OC(O)NR^{10a}R^{10b} or optionally substituted 5 to 10-membered heteroaryl; wherein
   R^{10a} and R^{10b} are each independently hydrogen, alkyl, or PEG;
   R^{10c} is alkyl, PEG, cycloalkyl, heterocycloalkyl, or phenyl; and
n₁ and m₁ are each independently 0 or 1.

In some embodiments, provided herein is a transcription modulator molecule having a structure of Formula (Ia), or a pharmaceutically acceptable salt thereof:

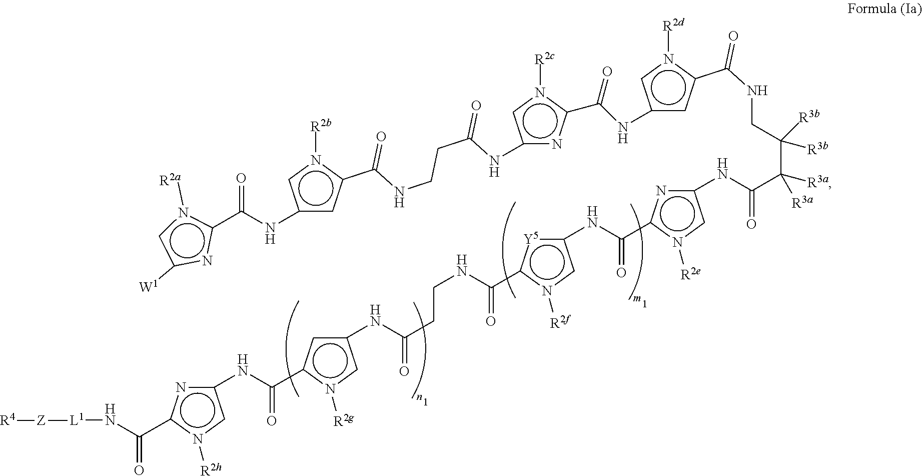

wherein:
W¹ is hydrogen;
each Y⁵ is independently CH or N;
L¹ is C₁-C₂₀ alkylene;
Z is absent or —C(O)—;
R⁴ is —NR^{4a}R^{4b}; wherein
   R^{4a} is hydrogen, optionally substituted C₁-C₂₀ alkyl, or optionally substituted C₁-C₂₀ heteroalkyl;
   R^{4b} is optionally substituted C₁-C₂₀ alkyl, optionally substituted C₁-C₂₀ aminoalkyl, optionally substituted C₁-C₂₀ haloalkyl, optionally substituted C₁-C₂₀ heteroalkyl, optionally substituted C₁-C₂₀ hydroxyalkyl, optionally substituted C₃-C₅ cycloalkyl, optionally substituted 4 to 8-membered heterocycloalkyl, optionally substituted phenyl, or optionally substituted 5 to 10-membered heteroaryl; or
   R^{4a} and R^{4b} together with the nitrogen to which they are attached form an optionally substituted 4 to 8-membered heterocycloalkyl;
each R^{2a}, R^{2b}, R^{2c}, R^{2d}, R^{2e}, R^{2f}, R^{2g}, and R^{2h} is independently hydrogen, optionally substituted C₁-C₁₀ alkyl, optionally substituted C₁-C₁₀ heteroalkyl, optionally substituted C₁-C₁₀ haloalkyl, or optionally substituted PEG₁₋₁₀; each of which is optionally substituted with one or more R¹⁰;
each R^{3a} and R^{3b} is independently hydrogen, —NR^{11a}R^{11b}, or —NHC(O)R¹², wherein
   R^{11a} and R^{11b} are each independently hydrogen, alkyl, or PEG;
   R¹² is alkyl, PEG, cycloalkyl, heterocycloalkyl, or phenyl;
or two R^{3a} or two R^{3b} together with the carbon atom to which they are attached form a C₃-C₆ cycloalkyl or 4 to 6-membered heterocycloalkyl;
each R¹⁰ is independently —CN, —OH, —OR^{10a}, —N₃, —NR^{10a}R^{10b}, —CO(O)R^{10c}, —C(O)OR^{10c}, —C(O)NR^{10a}R^{10b}, —NHC(O)R^{10c}, —NHC(O)OR^{10c}, —OC(O)NR^{10a}R^{10b}, or optionally substituted 5 to 10-membered heteroaryl; wherein
   R^{10a} and R^{10b} are each independently hydrogen, alkyl, or PEG;
   R^{10c} is alkyl, PEG, cycloalkyl, heterocycloalkyl, or phenyl; and
n₁ and m₁ are each independently 0 or 1.

In some embodiments, provided herein is a transcription modulator molecule having a structure of Formula (Ia), or a pharmaceutically acceptable salt thereof:

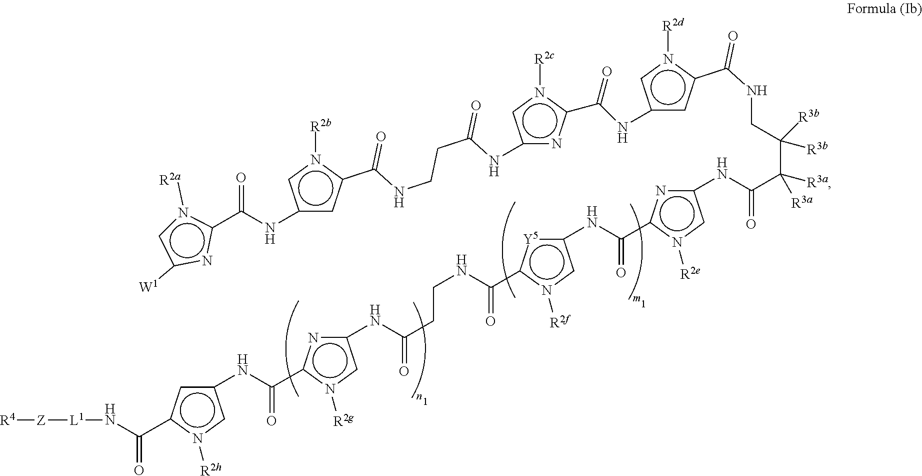

wherein:
W¹ is hydrogen or —N═C(N(R^{1e})₂)₂, wherein each R^{1e} is independently hydrogen or C₁-C₃ alkyl;
each Y⁵ is CH or N;
L¹ is C₁-C₂₀ alkylene or C₂-C₂₀ heteroalkylene;
Z is absent, —C(O)—, or —C(═NH)—;
R⁴ is C₁-C₆ alkyl, —OR^{4b}, or —NR^{4a}R^{4b}; wherein
   R^{4a} is hydrogen, optionally substituted C₁-C₂₀ alkyl, or optionally substituted C₁-C₂₀ heteroalkyl;
   R^{4b} is optionally substituted C₁-C₂₀ alkyl, optionally substituted C₂-C₂₀ alkenyl, optionally substituted C₂-C₂₀ alkynyl, optionally substituted C₁-C₂₀ aminoalkyl, optionally substituted C₁-C₂₀ haloalkyl, optionally substituted C₁-C₂₀ heteroalkyl, optionally substituted C₁-C₂₀ hydroxyalkyl, optionally substituted C₃-C₇ cycloalkyl, optionally substituted 4 to 8-membered heterocycloalkyl, optionally substituted phenyl, or optionally substituted 5 to 10-membered heteroaryl; or
   R^{4a} and R^{4b} together with the nitrogen to which they are attached form an optionally substituted 4 to 8-membered heterocycloalkyl which is partially or fully unsaturated;
each R^{2a}, R^{2b}, R^{2c}, R^{2d}, R^{2e}, R^{2f}, R^{2g}, and R^{2h} is independently hydrogen, optionally substituted C₁-C₅₀ alkyl, optionally substituted C₂-C₅₀ alkenyl, optionally substituted C₂-C₅₀ alkynyl, optionally substituted C₁-C₅₀ heteroalkyl, optionally substituted C₂-C₅₀ heteroalkenyl, optionally substituted C₂-C₅₀ heteroalkynyl, optionally substituted C₁-C₅₀ haloalkyl, optionally substituted C₃-C₈ cycloalkyl, optionally substituted 3 to 8-membered heterocycloalkyl, or optionally substituted PEG₁₋₅₀; each of which is optionally substituted with one or more R¹⁰.
each R^{3a} and R^{3b} is independently hydrogen, halogen, —NR^{11a}R^{11b}, or —NHC(O)R¹², wherein
   R^{11a} and R^{11b} are each independently hydrogen, alkyl, or PEG;
   R¹² is alkyl, PEG, cycloalkyl, heterocycloalkyl, or phenyl;
or two R^{3a} or two R^{3b} together with the carbon atom to which they are attached form a C₃-C₆ cycloalkyl or 4 to 6-membered heterocycloalkyl;
each R¹⁰ is independently —CN, —OH, —OR^{10a}, —N₃, —NR^{10a}R^{10b}, —CO(O)R^{10c}, —C(O)OR^{10c}, —C(O)NR^{10a}R^{10b}, —NHC(O)R^{10c}, —NHC(O)OR^{10c}, —OC(O)NR^{10a}R^{10b}, or optionally substituted 5 to 10-membered heteroaryl; wherein
   R^{10a} and R^{10b} are each independently hydrogen, alkyl, or PEG;
   R^{10c} is alkyl, PEG, cycloalkyl, heterocycloalkyl, or phenyl; and
n₁ and m₁ are each independently 0 or 1.

In some embodiments, the molecule of Formula (I) has the structure of Formula (Ib), or a pharmaceutically acceptable salt thereof:

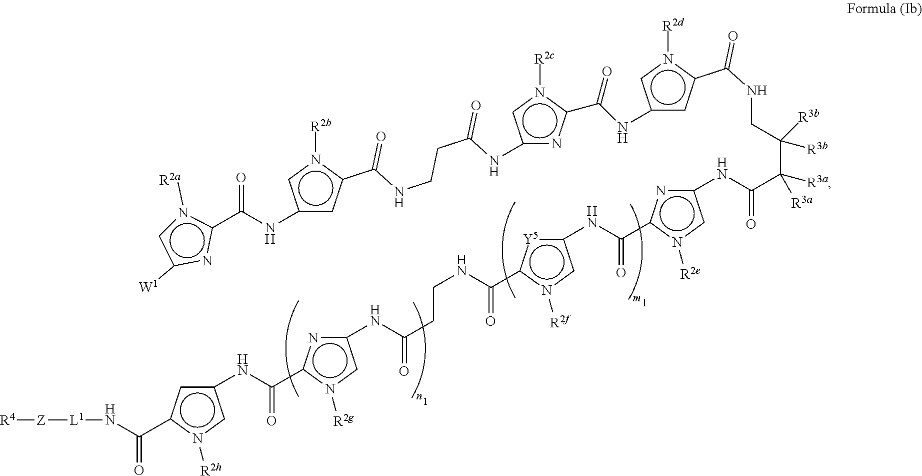

wherein:
W¹ is hydrogen;
each Y⁵ is independently CH or N;
L¹ is C₁-C₂₀ alkylene;
Z is absent or —C(O)—;
R⁴ is —NR^{4a}R^{4I}; wherein
   R^{4a} is hydrogen, optionally substituted C₁-C₂₀ alkyl, or optionally substituted C₁-C₂₀ heteroalkyl;
   R^{4b} is optionally substituted C₁-C₂₀ alkyl, optionally substituted C₁-C₂₀ aminoalkyl, optionally substituted C₁-C₂₀ haloalkyl, optionally substituted C₁-C₂₀ heteroalkyl, optionally substituted C₁-C₂₀ hydroxyalkyl, optionally substituted C₃-C₈ cycloalkyl, optionally substituted 4 to 8-membered heterocycloalkyl, optionally substituted phenyl, or optionally substituted 5 to 10-membered heteroaryl; or
   R^{4a} and R^{4b} together with the nitrogen to which they are attached form an optionally substituted 4 to 8-membered heterocycloalkyl;
each R^{2a}, R^{2b}, R^{2c}, R^{2d}, R^{2e}, R^{2f}, R^{2g}, and R^{2h} is independently hydrogen, optionally substituted C₁-C₁₀ alkyl, optionally substituted C₁-C₁₀ heteroalkyl, optionally substituted C₁-C₁₀ haloalkyl, or optionally substituted PEG₁₋₁₀; each of which is optionally substituted with one or more R¹⁰;
each R^{3a} and R^{3b} is independently hydrogen, —NR^{11a}R^{11b}, or —NHC(O)R¹², wherein
   R^{11a} and R^{11b} are each independently hydrogen, alkyl, or PEG;
   R¹² is alkyl, PEG, cycloalkyl, heterocycloalkyl, or phenyl;
or two R^{3a} or two R^{3b} together with the carbon atom to which they are attached form a C₃-C₆ cycloalkyl or 4 to 6-membered heterocycloalkyl;
each R¹⁰ is independently —CN, —OH, —OR^{10a}, —N₃, —NR^{10a}R^{10b}, —CO(O)R^{10c}, —C(O)OR^{10c}, —C(O)NR^{10a}R^{10b}, —NHC(O)R^{10c}, —NHC(O)OR^{10c}, —OC(O)NR^{10a}R^{10b}, or optionally substituted 5 to 10-membered heteroaryl; wherein
   R^{10a} and R^{10b} are each independently hydrogen, alkyl, or PEG;
   R^{10c} is alkyl, PEG, cycloalkyl, heterocycloalkyl, or phenyl; and
n₁ and m₁ are each independently 0 or 1.

In some embodiments of Formula (A), (A-1), (I), (Ia), or (Ib), L¹ is C₁-C₁₀ alkylene or C₂-C₁₀ heteroalkylene. In some embodiments, L¹ is C₁-C₁₀ alkylene, C₁-C₈ alkylene, C₁-C₆ alkylene, C₁-C₅ alkylene, C₁-C₄ alkylene, C₁-C₃ alkylene, or C₁-C₂ alkylene. In some embodiments, L¹ is C₁-C₄ alkylene. In some embodiments, L¹ is C₁-C₃ alkylene. In some embodiments, L¹ is C₁-C₂ alkylene. In some embodiments, L¹ is C₂-C₁₀ heteroalkylene, C₂-C₈ heteroalkylene, C₂-C₆ heterolkylene, C₂-C₅ heteroalkylene, or C₂-C₄ heteroalkylene. In some embodiments, L¹ is C₂-C₁₀ heteroalkylene. In some embodiments, L¹ is C₂-C₈ heteroalkylene. In some embodiments, L¹ is C₂-C₆ heterolkylene. In some embodiments, L¹ is C₂-C₅ heteroalkylene. In some embodiments, L¹ is C₂-C₄ heteroalkylene.

In some embodiments of Formula (A), (A-1), (I), (Ia), or (Tb), the heteroalkylene is polyethylene glycol. In some embodiments, L¹ is PEG₁₋₁₀. In some embodiments, L¹ is PEG₁₋₅₀. In some embodiments, L¹ is —(CH₂CH₂—O)_{y1}—, wherein y₁ is an integer in the range of 1-10. In some embodiments, y₁ is an integer in the range of 1-8. In some embodiments, y₁ is an integer in the range of 1-6. In some embodiments, y₁ is an integer in the range of 1-4. In some embodiments, y₁ is 1-2.

In some embodiments of Formula (A), (A-1), (I), (Ia), or (Ib), the heteroalkylene comprises —(CH₂)ₓ₃N(R^{a})(CH₂)ₓ₄—, wherein Ra is hydrogen or an optionally substituted C₁-C₆ alkyl; and each x₃ and x₄ is independently an integer in the range of 1-6.

In some embodiments of Formula (A), (A-1), (I), (Ia), or (Ib), Z is —C(O)—; and R⁴ is —OR^{4b}. In some embodiments, Z is —C(O)—; and R⁴ is —NR^{4a}R^{4b}. In some embodiments, Z is —C(═NH)—; and R⁴ is —NR^{4a}R^{4b}. In some embodiments, Z is absent; and R⁴ is —OR^{4b}. In some embodiments, Z is absent; and R⁴ is —NR^{4a}R^{4b}. In some embodiments, Z is —C(O)—; and R⁴ is C₁-C₆ alkyl. In some embodiments of Formula (A), (A-1), (I), (Ia), or (Ib), Z is absent; and R⁴ is C₁-C₆ alkyl.

In some embodiments of Formula (A), (A-1), (I), (Ia), or (Ib), R^{4a} is hydrogen, optionally substituted C₁-C₂₀ alkyl, or optionally substituted C₁-C₂₀ heteroalkyl. In some embodiments, R^{4a} is an optionally substituted C₁-C₂₀ alkyl or optionally substituted C₁-C₂₀ heteroalkyl. In some embodiments. R^{4a} is an optionally substituted C₁-C₂₀ alkyl. In some embodiments, R^{4a} is an optionally substituted C₁-C₁₅ alkyl. In some embodiments, R^{4a} is an optionally substituted C₁-C₁₀ alkyl. In some embodiments, R^{4a} is an optionally substituted C₁-C₂₀ heteroalkyl. In some embodiments, the heteroalkyl is polyethylene glycol (PEG). In some embodiments, R^{4a} is optionally substituted PEG₁₋₂₀. In some embodiments, R^{4a} is an optionally substituted PEG₁₋₁₅. In some embodiments, R^{4a} is optionally substituted PEG₁₋₁₀. In some embodiments, R^{4a} is hydrogen.

In some embodiments of Formula (A), (A-1), (I), (Ia), or (Ib), R^{4b} is optionally substituted C₁-C₂₀ alkyl, optionally substituted C₂-C₂₀ alkenyl, optionally substituted C₂-C₂₀ alkynyl, optionally substituted C₁-C₂₀ aminoalkyl, optionally substituted C₁-C₂₀ haloalkyl, optionally substituted, C₁-C₂₀ heteroalkyl, optionally substituted C₁-C₂₀ hydroxyalkyl, optionally substituted C₃-C₈ cycloalkyl, optionally substituted 4 to 8-membered heterocycloalkyl, optionally substituted phenyl, or optionally substituted 5 to 10-membered heteroaryl. In some embodiments, R^{4b} is optionally substituted C₁-C₂₀ alkyl, optionally substituted C₁-C₂₀ aminoalkyl, optionally substituted C₁-C₂₀ haloalkyl, optionally substituted C₁-C₂₀ heteroalkyl, optionally substituted C₁-C₂₀ hydroxyalkyl, optionally substituted C₃-C₈ cycloalkyl, optionally substituted 4 to 8-membered heterocycloalkyl, optionally substituted phenyl, or optionally substituted 5 to 10-membered heteroaryl.

In some embodiments of Formula (A), (A-1), (I), (Ia), or (Ib), R^{4b} is optionally substituted C₁-C₂₀ alkyl, optionally substituted C₁-C₂₀ aminoalkyl, optionally substituted C₁-C₂₀ haloalkyl, optionally substituted C₁-C₂₀ heteroalkyl, or optionally substituted C₁-C₂₀ hydroxyalkyl. In some embodiments, R^{4b} is optionally substituted C₁-C₂₀ alkyl or optionally substituted C₁-C₂₀ heteroalkyl. In some embodiments. R^{4b} is optionally substituted C₁-C₂₀ alkyl. In some embodiments, R^{4b} is optionally substituted C₁-C₁₅ alkyl. In some embodiments, R^{4b} is optionally substituted C₁-C₁₀ alkyl. In some embodiments, R^{4b} is optionally substituted C₁-C₂₀ heteroalkyl. In some embodiments, R^{4b} is optionally substituted C₁-C₁₅ heteroalkyl. In some embodiments, R^{4b} is optionally substituted C₁-C₁₀ heteroalkyl. In some embodiments, the heteroalkyl is polyethylene glycol (PEG). In some embodiments, R^{4b} is PEG₁₋₂₀. In some embodiments, R^{4b} is PEG₁₋₁₅. In some embodiments, R^{4b} is PEG₁₋₁₀.

In some embodiments of Formula (A), (A-1), (I), (Ia), or (Ib), R^{4b} is optionally substituted C₃-C₈ cycloalkyl, optionally substituted 4 to 8-membered heterocycloalkyl, optionally substituted phenyl, or optionally substituted 5 to 10-membered heteroaryl. In some embodiments, R^{4b} is optionally substituted C₃-C₅ cycloalkyl or optionally substituted 4 to 8-membered heterocycloalkyl. In some embodiments, R^{1b} is optionally substituted C₃-C₆ cycloalkyl or optionally substituted 4 to 6-membered heterocycloalkyl. In some embodiments, R^{4b} is optionally substituted C₃-C₆ cycloalkyl. In some embodiments, R^{4b} is cyclopentyl or cyclohexyl. In some embodiments, R^{4b} is optionally substituted 4 to 6-membered heterocycloalkyl. In some embodiments, R^{4b} is a 5 or 6-membered heterocycloalkyl. In some embodiments, R^{4b} is a piperidine, piperazine, or morpholine. In some embodiments, R^{4b} is a piperidine or piperazine. In some embodiments, R^{4b} is piperidine. In some embodiments, R^{4b} is piperazine.

In some embodiments of Formula (A), (A-1), (I), (Ia), or (Ib), R^{4a} and R^{4b} together with the nitrogen to which they are attached form an optionally substituted 4 to 8-membered heterocycloalkyl which is partially or fully unsaturated. In some embodiments, R^{4a} and R^{4b} together with the nitrogen to which they are attached form an optionally substituted 4 to 6-membered heterocycloalkyl. In some embodiments, R^{4a} and R^{4b} together with the nitrogen to which they are attached form an optionally substituted 4-membered heterocycloalkyl. In some embodiments, R^{4a} and R^{4b} together with the nitrogen to which they are attached form an optionally substituted 5-membered heterocycloalkyl. In some embodiments, R^{4a} and R^{4b} together with the nitrogen to which they are attached form an optionally substituted 6-membered heterocycloalkyl.

In some embodiments of Formula (A), (A-1), (I), (Ia), or (Ib), R^{4a} is hydrogen, optionally substituted C₁-C₂₀ alkyl, or optionally substituted C₁-C₂₀ heteroalkyl; and R^{4b} is optionally substituted C₁-C₂₀ alkyl or optionally substituted C₁-C₂₀ heteroalkyl. In some embodiments, R^{4a} is hydrogen, C₁-C₂₀ alkyl, or C₁-C₂₀ heteroalkyl; and R^{1b} is C₁-C₂₀ alkyl, or C₁-C₂₀ heteroalkyl.

In some embodiments of Formula (A), (A-1), (I), (Ia), or (Ib), R^{4a} is optionally substituted C₁-C₂₀ heteroalkyl; and R^{4b} is optionally substituted C₁-C₂₀ heteroalkyl. In some embodiments, each heteroalkyl is polyethylene glycol (PEG). In some embodiments, R^{4a} is hydrogen or PEG₁₋₂₀; and R^{4b} is PEG₁₋₂₀. In some embodiments, R^{4a} is PEG₁₋₂₀; and R⁴ⁱ is PEG₁₋₂₀.

In some embodiments of Formula (A), (A-1), (I), (Ia), or (Ib), R^{4a} is optionally substituted C₁-C₆ alkyl; and R^{4b} is optionally substituted C₁-C₆ alkyl. In some embodiments, R^{4a} is C₁-C₆ alkyl; and R^{1b} is C₁-C₆ alkyl.

In another aspect, provided herein is a transcription modulator molecule having a structure of Formula (II), or a pharmaceutically acceptable salt thereof:

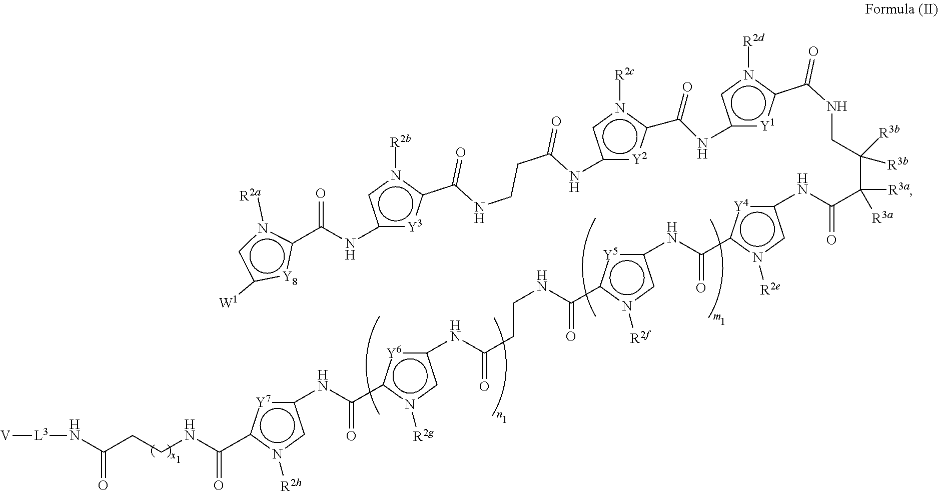

wherein:
W¹ is hydrogen or —N═C(N(R^{1e})₂)₂, wherein each R^{1e} is independently hydrogen or C₁-C₃ alkyl;
each Y¹, Y², Y³, Y⁴, Y⁵, Y⁶, Y⁷, and Y⁸ is independently N or CH;
L³ is C₁-C₂₀ alkylene, C₂-C₂₀ heteroalkylene, or AA₁₋₁₀; wherein
   each AA is independently a naturally occurring amino acid;
V is absent, optionally substituted C₃-C₈ cycloalkyl, optionally substituted 4 to 8-membered heterocycloalkyl, optionally substituted phenyl, or optionally substituted 5 to 10-membered heteroaryl;
each R^{2a}, R^{2b}, R^{2c}, R^{2d}, R^{2e}, R^{2f}, R^{2g}, and R^{2h} is independently hydrogen, optionally substituted C₁-C₅₀ alkyl, optionally substituted C₂-C₅₀ alkenyl, optionally substituted C₂-C₅₀ alkynyl, optionally substituted C₁-C₅₀ heteroalkyl, optionally substituted C₂-C₅₀ heteroalkenyl, optionally substituted C₂-C₅₀ heteroalkynyl, optionally substituted C₁-C₅₀ haloalkyl, optionally substituted C₃-C₈ cycloalkyl, optionally substituted 3 to 8-membered heterocycloalkyl, or optionally substituted PEG₁₋₅₀; each of which is optionally substituted with one or more R¹⁰;
each R^{3a} and R^{3b} is independently hydrogen, halogen, —NR^{11a}R^{11b}, or —NHC(O)R¹², wherein
   R^{11a} and R^{11b} are each independently hydrogen, alkyl, or PEG;
   R¹² is alkyl, PEG, cycloalkyl, heterocycloalkyl, or phenyl:
or two R^{3a} or two R^{3b} together with the carbon atom to which they are attached form a C₃-C₆ cycloalkyl or 4 to 6-membered heterocycloalkyl;
each R¹⁰ is independently —CN, —OH, —OR^{10a}, —N₃, —NR^{10a}R^{10b}, —CO(O)R^{10c}, —C(O)OR^{10c}, —C(O)NR^{10a}R^{10b}, —NHC(O)R^{10c}, —NHC(O)OR^{10c}, —OC(O)NR^{10a}R^{10b}, or optionally substituted 5 to 10-membered heteroaryl; wherein
   R^{10a} and R^{10b} are each independently hydrogen, alkyl, or PEG;
   R^{10c} is alkyl, PEG, cycloalkyl, heterocycloalkyl, or phenyl;
n₁ and m₁ are each independently 0 or 1; and
x₁ is 0-10.

In some embodiments, the molecule of Formula (II) has the structure of Formula (IIa), or a pharmaceutically acceptable salt thereof:

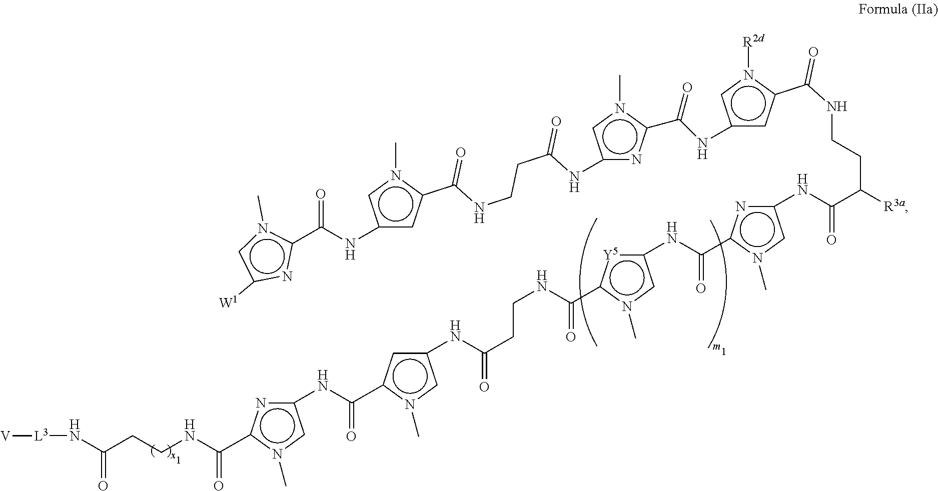

wherein:
W¹ is hydrogen or —N═C(N(R^{1e})₂)₂, wherein each R^{1e} is independently hydrogen or C₁-C₃ alkyl;
each Y⁵ is independently N or CH;
L³ is C₁-C₂₀ alkylene, C₂-C₂₀ heteroalkylene, or AA₁₋₁₀; wherein
   each AA is independently a naturally occurring amino acid;
V is absent, optionally substituted C₃-C₈ cycloalkyl, optionally substituted 4 to 8-membered heterocycloalkyl, optionally substituted phenyl, or optionally substituted 5 to 10-membered heteroaryl;
R^{2d} is hydrogen, optionally substituted C₁-C₅₀ alkyl, optionally substituted C₂-C₅₀ alkenyl, optionally substituted C₂-C₅₀ alkynyl, optionally substituted C₁-C₅₀ heteroalkyl, optionally substituted C₂-C₅₀ heteroalkenyl, optionally substituted C₂-C₅₀ heteroalkynyl, optionally substituted C₁-C₅₀ haloalkyl, optionally substituted C₃-C₈ cycloalkyl, optionally substituted 3 to 8-membered heterocycloalkyl, or optionally substituted PEG₁₋₅₀; each of which is optionally substituted with one or more R¹⁰;
R^{3a} is hydrogen, halogen, —NR^{11a}R^{11b}, or —NHC(O)R¹², wherein
   R^{11a} and R^{11b} are each independently hydrogen, alkyl, or PEG;
   R¹² is alkyl, PEG, cycloalkyl, heterocycloalkyl, or phenyl;
each R¹⁰ is independently —CN, —OH, —OR^{10a}, —N₃, —NR^{10a}R^{10b}, —CO(O)R^{10c}, —C(O)OR^{10c}, —C(O)NR^{10a}R^{10b}, —NHC(O)R^{10c}, —NHC(O)OR^{10c}, —OC(O)NR^{10a}R^{10b}, or optionally substituted 5 to 10-membered heteroaryl; wherein
   R^{10a} and R^{10b} are each independently hydrogen, alkyl, or PEG;
   R^{10c} is alkyl, PEG, cycloalkyl, heterocycloalkyl, or phenyl;
m₁ is 0 or 1; and
x₁ is 0-10.

In some embodiments of Formula (II) or (IIa), L³ is C₁-C₁₀ alkylene or C₂-C₁₀ heteroalkylene. In some embodiments, L³ is C₁-C₁₀ alkylene, C₁-C₈ alkylene, C₁-C₆ alkylene, C₁-C₅ alkylene, C₁-C₄ alkylene, C₁-C₃ alkylene, or C₁-C₂ alkylene. In some embodiments, L³ is C₁-C₄ alkylene. In some embodiments. L³ is C₁-C₃ alkylene. In some embodiments, L³ is C₁-C₂ alkylene. In some embodiments, L³ is C₂-C₁₀ heteroalkylene, C₂-C₅ heteroalkylene, C₂-C₆ heterolkylene, C₂-C₅ heteroalkylene, or C₂-C₄ heteroalkylene. In some embodiments, L³ is C₂-C₁₀ heteroalkylene. In some embodiments, L³ is C₂-C₅ heteroalkylene. In some embodiments, L³ is C₂-C₆ heterolkylene. In some embodiments, L³ is C₂-C₅ heteroalkylene. In some embodiments, L³ is C₂-C₄ heteroalkylene.

In some embodiments of Formula (II) or (IIa), the heteroalkylene is polyethylene glycol. In some embodiments, L³ is PEG₁₋₁₀. In some embodiments, L³ is PEG₁₋₈. In some embodiments, L³ is —(CH₂CH₂—O)_{y1}—, wherein y₁ is an integer in the range of 1-10. In some embodiments, y₁ is an integer in the range of 1-8. In some embodiments, y₁ is an integer in the range of 1-6. In some embodiments, y₁ is an integer in the range of 1-4. In some embodiments, y₁ is 1-2.

In some embodiments of Formula (II) or (IIa), the heteroalkylene of L³ comprises —(CH₂)₃N(R^{a})(CH₂)ₓ₄—, wherein Ra is hydrogen or an optionally substituted C₁-C₆ alkyl; and each x₃ and x₄ is independently an integer in the range of 1-6.

In some embodiments of Formula (II) or (IIa), L³ is AA₁₋₁₀, wherein each AA is independently a naturally occurring amino acid. In some embodiments, L³ is AA₁₋₈. In some embodiments, L³ is AA₁₋₆. In some embodiments, L³ is AA₁₋₅. In some embodiments, L³ is AA₁₋₄. In some embodiments, L³ is AA₁₋₃. In some embodiments, L³ is AA₁₋₂. In some embodiments, each AA is the same or different. In some embodiments, each AA is the same. In some embodiments, each AA is different.

In some embodiments of Formula (II) or (IIa), V is optionally substituted C₃-C₈ cycloalkyl, optionally substituted 4 to 8-membered heterocycloalkyl, optionally substituted phenyl, or optionally substituted 5 to 10-membered heteroaryl. In some embodiments, V is optionally substituted C₃-C₈ cycloalkyl or optionally substituted 4 to 8-membered heterocycloalkyl. In some embodiments. V is an optionally substituted phenyl or optionally substituted 5 to 10-membered heteroaryl. In some embodiments, V is an optionally substituted phenyl or optionally substituted 6-membered heteroaryl. In some embodiments, V is an optionally substituted phenyl. In some embodiments, V is an optionally substituted 6-membered heteroaryl.

In some embodiments of Formula (II) or (IIa), V is absent.

In some embodiments of Formula (II) or (IIa), V is an optionally substituted C₃-C₈ cycloalkyl. In some embodiments, V is an optionally substituted C₃-C₆ cycloalkyl. In some embodiments, V is an optionally substituted cyclopentyl or optionally substituted cyclohexyl. In some embodiments, V is cyclopentyl. In some embodiments, V is cyclohexyl.

In some embodiments of Formula (II) or (IIa), V is an optionally substituted 4 to 8-membered heterocycloalkyl. In some embodiments, V is an optionally substituted 4 to 6-membered heterocycloalkyl. In some embodiments, V is an optionally substituted 6-membered heterocycloalkyl. In some embodiments, V is an optionally substituted piperazine, optionally substituted piperidine, or optionally substituted morpholine. In some embodiments, V is an optionally substituted piperazine. In some embodiments, V is an optionally substituted piperidine. In some embodiments, V is an optionally substituted morpholine.

In some embodiments of Formula (II) or (IIa), V has the structure of Formula (C), or a pharmaceutically acceptable salt thereof:

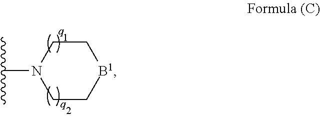

wherein:
B¹ is —CR^{5a}R^{5b}—, —O—, —NR^{5b}—, —S(O)—, —S(O)₂—, or —S—; or B¹ is

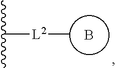

wherein
R^{5a} is hydrogen, —OH, or optionally substituted C₁-C₂₀ alkyl;
R^{5b} is hydrogen, optionally substituted C₁-C₂₀ alkyl, optionally substituted C₂-C₂₀ alkenyl, optionally substituted C₂-C₂₀ alkynyl, optionally substituted C₁-C₂₀ heteroalkyl, —C(O)OR⁶, or —C(O)R⁶; or
R^{5a} and R^{5b} together with the nitrogen atom to which they are attached form an optionally substituted 4 to 8-membered heterocycloalkyl;
R⁶ is hydrogen, optionally substituted C₁-C₂₀ alkyl, optionally substituted C₁-C₁₀ haloalkyl, optionally substituted C₃-C₆ cycloalkyl, optionally substituted 3 to 6-membered heterocycloalkyl, or optionally substituted phenyl;
ring B is absent, optionally substituted C₃-C₆ cycloalkyl, optionally substituted 4 to 6-membered heterocycloalkyl, optionally substituted phenyl, or optionally substituted 5 to 10-membered heteroaryl;
L² is absent, C₁-C₄ alkylene, C₂-C₄ alkynelene, or C₂-C₄ alkynylene; and
q₁ and q₂ are each independently 0, 1, or 2.

In some embodiments of Formula (II) or (IIa), V has the structure of Formula (C-1), or a pharmaceutically acceptable salt thereof:

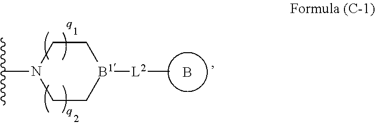

wherein:
ring B is optionally substituted C₃-C₆ cycloalkyl, optionally substituted 4 to 6-membered heterocycloalkyl, optionally substituted phenyl, or optionally substituted 5 to 10-membered heteroaryl;
L² is absent, C₁-C₄ alkylene, C₂-C₄ alkynelene, or C₂-C₄ alkynylene;
B¹ is CH or N; and
q₁ and q₂ are each independently 0, 1, or 2.

In some embodiments of Formula (II) or (IIa), V has the structure of Formula (C-2), or a pharmaceutically acceptable salt thereof:

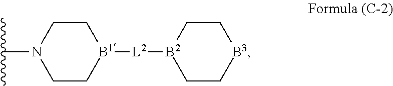

wherein:
B¹ and B² are each independently CH or N; and
B³ is —CR^{7a}R^{7b}—, —O—, —S—, —S(O)—, —S(O)₂—, or —NR^{7b}—; wherein
   R^{7a} is hydrogen or optionally substituted C₁-C₂₀ alkyl;
   R^{7b} is hydrogen, optionally substituted C₁-C₂₀ alkyl, optionally substituted C₂-C₂₀ alkenyl, optionally substituted C₂-C₂₀ alkynyl, optionally substituted C₁-C₂₀ heteroalkyl, —C(O)OR^{B}, or —C(O)R⁸.
   R⁸ is hydrogen, optionally substituted C₁-C₂₀ alkyl, optionally substituted C₁-C₁₀ haloalkyl, optionally substituted PEG₁₋₂₀, optionally substituted C₃-C₆ cycloalkyl, optionally substituted 3 to 6-membered heterocycloalkyl, or optionally substituted phenyl; and
L² is absent, C₁-C₄ alkylene, C₂-C₄ alkynelene, or C₂-C₄ alkynylene.

In some embodiments of Formula (C-1) or (C-2), B¹ is CH or N. In some embodiments, B¹ is CH. In some embodiments, B¹ is N.

In some embodiments of formula (C-2), B¹ is CH and B² is N. In some embodiments, B¹ is N and B² is CH.

In some embodiments of Formula (II) or (IIa), V has the structure of Formula (C-3), or a pharmaceutically acceptable salt thereof:

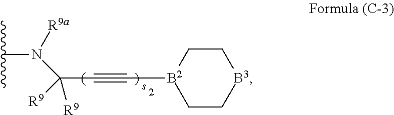

wherein:
B² is CH or N;
B³ is —CR^{7a}R^{7b}—, —O—, —S—, —S(O)—, —S(O)₂—, or —NR^{7b}—; wherein
   R^{7a} is hydrogen or optionally substituted C₁-C₂₀ alkyl;
   R^{7b} is hydrogen, optionally substituted C₁-C₂₀ alkyl, optionally substituted C₂-C₂₀ alkenyl, optionally substituted C₂-C₂₀ alknyl, optionally substituted C₁-C₂₀ heteroalkyl, —C(O)OR^{B}, or —C(O)R⁸; and
   R⁸ is hydrogen, optionally substituted C₁-C₂₀ alkyl, optionally substituted C₁-C₁₀ haloalkyl, optionally substituted PEG₁₋₂₀, optionally substituted C₃-C₆ cycloalkyl, optionally substituted 3 to 6-membered heterocycloalkyl, or optionally substituted phenyl;
R^{9a} is hydrogen, optionally substituted C₁-C₂₀ alkylene, or optionally substituted PEG₁₋₂₀;
each R⁹ is independently hydrogen or C₁-C₃ alkyl; and
s₂ is 1-3.

In some embodiments of Formula (C-2) or (C-3), B² is CH or N. In some embodiments, B² is CH. In some embodiments, B² is N.

In some embodiments of Formula (C-2) or (C-3), B³ is —CR^{2a}R^{2b}— or —O—. In some embodiments, B³ is —CR^{7a}R^{7b}—. In some embodiments, B³ is —O—. In some embodiments, B³ is —S—, —S(O)—, or —S(O)₂—. In some embodiments, B³ is —NR^{7b}.

In some embodiments of Formula (C-2) or (C-3), R^{7a} is an optionally substituted C₁-C₂₀ alkyl. In some embodiments, R^{7a} is hydrogen.

In some embodiments of Formula (C-2) or (C-3), R^{7b} is hydrogen, optionally substituted C₁-C₂₀ alkyl, optionally substituted C₂-C₂₀ alkenyl, optionally substituted C₂-C₂₀ alkynyl, or optionally substituted C₁-C₂₀ heteroalkyl. In some embodiments, R^{7b} is optionally substituted C₁-C₂₀ alkyl. In some embodiments, R^{7b} is an optionally substituted C₂-C₂₀ alkenyl. In some embodiments, R^{7b} is an optionally substituted C₂-C₂₀ alkynyl. In some embodiments of Formula (C-2) or (C-3), R^{7b} is —C(O)OR⁸ or —C(O)R⁸. In some embodiments, R^{7b} is hydrogen.

In some embodiments of Formula (C-2) or (C-3), R⁸ is an optionally substituted C₁-C₂₀ alkyl. In some embodiments, R⁸ is an optionally substituted PEG₁₋₂₀. In some embodiments, R⁸ is an optionally substituted phenyl. In some embodiments, R⁸ is hydrogen.

In some embodiments of Formula (C-3), R^{9a} is optionally substituted C₁-C₂₀ alkylene or optionally substituted PEG₁₋₂₀. In some embodiments, R^{9a} is an optionally substituted C₁-C₂₀ alkylene. In some embodiments, R^{9a} is an optionally substituted PEG₁₋₂₀. In some embodiments, R^{9a} is hydrogen.

In some embodiments of Formula (C-3), each R⁹ is independently hydrogen or C₁-C₃ alkyl. In some embodiments, each R⁹ is independently C₁-C₃ alkyl. In some embodiments, each R⁹ is independently hydrogen.

In some embodiments of Formula (C-3), s₂ is 1 or 2. In some embodiments, s₂ is 3. In some embodiments, s₂ is 2. In some embodiments, s₂ is 1.

In some embodiments of Formula (II) or (IIa), V has the structure of Formula (C-4), or a pharmaceutically acceptable salt thereof:

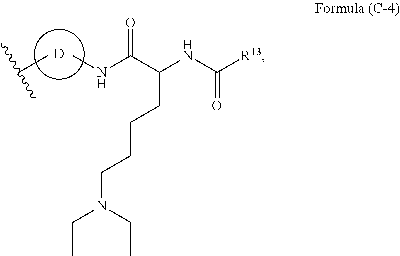

wherein;
ring D is absent or phenyl; and
R¹³ is C₁-C₆ alkyl, C₃-C₈ cycloalkyl, 4 to 8-membered heterocycloalkyl, or phenyl.

In some embodiments of Formula (C-4), ring D is phenyl. In some embodiments, ring D is absent.

In some embodiments of Formula (C-4), R¹³ is C₁-C₆ alkyl. In some embodiment, R¹³ is C₃-C₈ cycloalkyl. In some embodiments, R¹³ is a C₃-C₆ cycloalkyl. In some embodiments, R¹³ is 4 to 8-membered heteroalkyl. In some embodiments, R¹³ is a 4 to 6-membered heterocycloalkyl.

In some embodiments of Formula (II) or (IIa), V has the structure of Formula (C-5), or a pharmaceutically acceptable salt thereof:

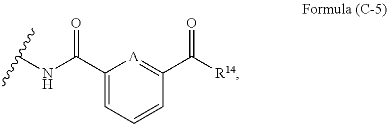

wherein,
A is CH or N; and
R¹⁴ is OH or NH₂.

In some embodiments of Formula (C-5), A is CH. In some embodiment, A is N.

In some embodiment of Formula (C-5), R¹⁴ is OH. In some embodiments, R¹⁴ is NH₂.

In some embodiments of Formula (A), (A-1), or (I), L¹ is C₁-C₁₀ alkylene and R⁴ is —NR^{4a}R^{4b}, wherein R^{4a} and R^{4b} together with the nitrogen to which they are attached form an optionally substituted 4 to 8-membered heterocycloalkyl. In some embodiments, L¹ is C₁-C₄ alkylene and R⁴ is —NR^{4a}R^{4b}, wherein R^{4a} and R^{4b} together with the nitrogen to which they are attached form an optionally substituted 4-membered heterocycloalkyl. In some embodiments, L¹ is C₁-C₄ alkylene and R⁴ is —NR^{4a}R^{4b}, wherein R^{4a} and R^{4b} together with the nitrogen to which they are attached form an optionally substituted 5-membered heterocycloalkyl. In some embodiments, L¹ is C₁-C₄ alkylene and R⁴ is —NR^{4a}R^{4b}, wherein R^{4a} and R^{4b} together with the nitrogen to which they are attached form an optionally substituted 6-membered heterocycloalkyl. In some embodiments, L¹ is C₁-C₄ alkylene and R⁴ is —NR^{4a}R^{4b}, wherein R^{4a} and R^{4b} together with the nitrogen to which they are attached form an optionally substituted 7-membered heterocycloalkyl.

In some embodiments, provided herein is a transcription modulator molecule having a structure of Formula (III), or a pharmaceutically acceptable salt thereof:

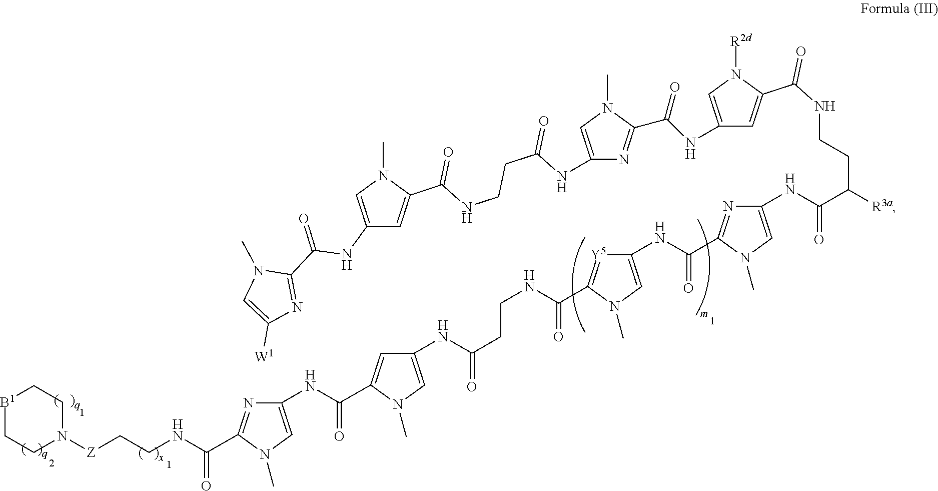

wherein:
B¹ is —CR^{5a}R^{5b}—, —O—, —NR^{5b}—, —S—, —S(O)—, or —S(O)₂—; or B¹ is

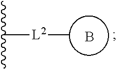

wherein
R^{5a} is hydrogen, —OH, or optionally substituted C₁-C₂₀ alkyl;
R^{5b} is hydrogen, optionally substituted C₁-C₂₀ alkyl, optionally substituted C₂-C₂₀ alkenyl, optionally substituted C₂-C₂₀ alkynyl, optionally substituted C₁-C₂₀ heteroalkyl, —C(O)R⁶, or C(O)R⁶; or
R^{5a} and R^{5b} together with the nitrogen atom to which they are attached form an optionally substituted 4 to 8-membered heterocycloalkyl;
R⁶ is hydrogen, optionally substituted C₁-C₂₀ alkyl, optionally substituted C₁-C₁₀ haloalkyl, optionally substituted C₃-C₆ cycloalkyl, optionally substituted 3 to 6-membered heterocycloalkyl, or optionally substituted phenyl;
ring B is absent, optionally substituted C₃-C₆ cycloalkyl, optionally substituted 4 to 6-membered heterocycloalkyl, optionally substituted phenyl, or optionally substituted 5 to 10-membered heteroaryl;
L² is absent, C₁-C₄ alkylene, C₂-C₄ alkynelene, or C₂-C₄ alkynylene;
W¹ is hydrogen or —N═C(N(R^{e})₂)₂, wherein each Re is independently hydrogen or C₁-C₃ alkyl;
Z is absent or C(O);
each Y⁵ is independently N or CH;
R^{2d} is hydrogen, optionally substituted C₁-C₅₀ alkyl, optionally substituted C₂-C₅₀ alkenyl, optionally substituted C₂-C₅₀ alkynyl, optionally substituted C₁-C₅₀ heteroalkyl. optionally substituted C₂-C₅₀ heteroalkenyl, optionally substituted C₂-C₅₀ heteroalkynyl, optionally substituted C₁-C₅₀ haloalkyl, optionally substituted C₃-C₈ cycloalkyl, optionally substituted 3 to 8-membered heterocycloalkyl, or optionally substituted PEG₁₋₅₀; each of which is optionally substituted with one or more R¹⁰;
R^{3a} is hydrogen, halogen, —NR^{11a}R^{11b}, or —NHC(O)R¹², wherein
   R^{11a} and R^{11b} are each independently hydrogen, alkyl, or PEG;
   R¹² is alkyl, PEG, cycloalkyl, heterocycloalkyl, or phenyl;
each R¹⁰ is independently —CN, —OH, —OR^{10a}, —N₃, —NR^{10a}R^{10b}, —CO(O)R^{10c}, —C(O)OR^{10c}, —C(O)NR^{10a}R^{10b}, —NHC(O)R^{10c}, —NHC(O)OR^{10c}, —OC(O)NR^{10a}R^{10b}, or optionally substituted 5 to 10-membered heteroaryl; wherein
   R^{10a} and R^{10b} are each independently hydrogen, alkyl, or PEG;
   R^{10c} is alkyl, PEG, cycloalkyl, heterocycloalkyl, or phenyl;
m₁ is 0 or 1;
q₁ and q₂ are each independently 0-2; and
x₁ is 0-10.

In some embodiments of Formula (II), (IIa), or (III), x₁ is 0-10. In some embodiments, x₁ is 0-8. In some embodiments, x₁ is 0-6. In some embodiments, x₁ is 1-10. In some embodiments, x₁ is 1-8. In some embodiments, x₁ is 1-6. In some embodiments, x₁ is 1-5. In some embodiments, x₁ is 1-4. In some embodiments, x₁ is 1-3. In some embodiments, x₁ is 1-2. In some embodiments, x₁ is 0, 1, 2, 3, 4, 5, or 6. In some embodiments, x₁ is 1. In some embodiments, x₁ is 2. In some embodiments, x₁ is 3. In some embodiments, x₁ is 4. In some embodiments, x₁ is 5. In some embodiments, x₁ is 6.

In another aspect, provided herein is a transcription modulator molecule having a structure of Formula (IV), or a pharmaceutically acceptable salt thereof:

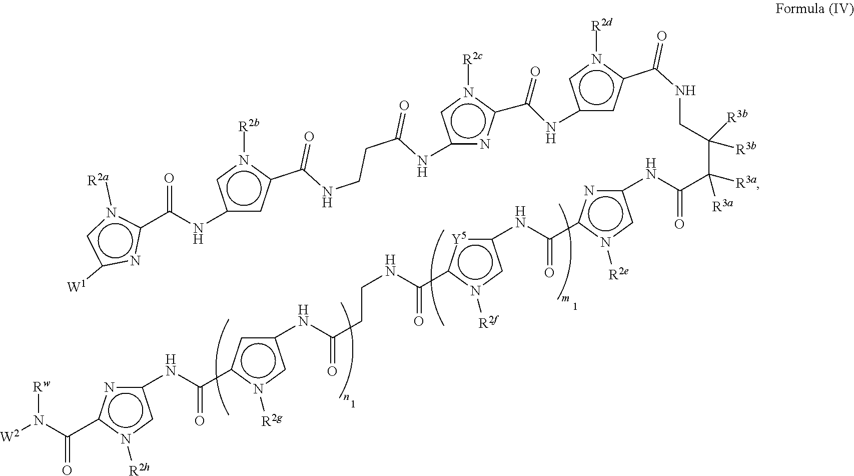

wherein:
W¹ is hydrogen or —N═C(N(R^{1e})₂)₂, wherein each R^{1e} is independently hydrogen or C₁-C₃ alkyl;
W² is hydrogen, optionally substituted C₁-C₂₀ alkyl, or optionally substituted C₁-C₂₀ heteroalkyl;
R^{W} is hydrogen or C₁-C₂₀ alkyl;
or W² and R^{W} together with the nitrogen to which they are attached form an optionally substituted 4 to 8-membered heterocycloalkyl which is partially or fully unsaturated;
each Y⁵ is independently N or CH;
each R^{2a}, R^{2b}, R^{2c}, R^{2d}, R^{2e}, R^{2f}, R^{2g}, and R^{2h} is independently hydrogen, optionally substituted C₁-C₅₀ alkyl, optionally substituted C₂-C₅₀ alkenyl, optionally substituted C₂-C₅₀ alkynyl, optionally substituted C₁-C₅₀ heteroalkyl, optionally substituted C₂-C₅₀ heteroalkenyl, optionally substituted C₂-C₅₀ heteroalkynyl, optionally substituted C₁-C₅₀ haloalkyl, optionally substituted C₃-C₈ cycloalkyl, optionally substituted 3 to 8-membered heterocycloalkyl, or optionally substituted PEG₁₋₅₀; each of which is optionally substituted with one or more R¹⁰;
each R^{3a} and R^{3b} is independently hydrogen, halogen, —NR^{11a}R^{11b}, or —NHC(O)R¹², wherein
   R^{11a} and R^{11b} are each independently hydrogen, alkyl, or PEG;
   R¹² is alkyl, PEG, cycloalkyl, heterocycloalkyl, or phenyl;
or two R^{3a} or two R^{3b} together with the carbon atom to which they are attached form a C₃-C₆ cycloalkyl or 4 to 6-membered heterocycloalkyl;
each R¹⁰ is independently —CN, —OH, —OR^{10a}—N₃, —NR^{10a}R^{10b}, —CO(O)R^{10c}, —C(O)OR^{10c}, —C(O)NR^{10a}R^{10b}, —NHC(O)R^{10c}, —NHC(O)OR^{10c}, —OC(O)NR^{10a}R^{10b}, or optionally substituted 5 to 10-membered heteroaryl; wherein
   R^{10a} and R^{10b} are each independently hydrogen, alkyl, or PEG;
   R^{10c} is alkyl, PEG, cycloalkyl, heterocycloalkyl, or phenyl; and
n₁ and m₁ are each independently 0 or 1.

In some embodiments of Formula (A), (A-1), or (IV), W² is optionally substituted C₁-C₂₀ alkyl or optionally substituted C₁-C₂₀ heteroalkyl. In some embodiments, W² is optionally substituted C₁-C₂₀ alkyl. In some embodiments, W² is optionally substituted C₁-C₂₀ heteroalkyl. In some embodiments of Formula (A), (A-1), or (IV), W² is C₁-C₂₀ alkyl. In some embodiments, W² is C₁-C₁₅ alkyl. In some embodiments, W² is C₁-C₁₀ alkyl. In some embodiments, W² is C₁-C₈ alkyl. In some embodiments, W² is C₁-C₆ alkyl. In some embodiments, W² is C₁-C₃ alkyl. In some embodiments, W² is methyl, ethyl, n-propyl, or isopropyl. In some embodiments, W² is C₁-C₃ alkyl. In some embodiments, W² is methyl. In some embodiments, W² is C₁-C₃ alkyl. In some embodiments, W² is ethyl. In some embodiments, W² is methyl, ethyl, n-propyl.

In some embodiments of Formula (A), (A-1), or (IV), W² is C₁-C₂₀ heteroalkyl. In some embodiments, W² is C₁-C₁₅ heteroalkyl. In some embodiments, W² is C₁-C₁₀ heteroalkyl. In some embodiments, W² is C₁-C₈ heteroalkyl. In some embodiments, W² is optionally substituted C₁-C₆ heteroalkyl. In some embodiments, the heteroalkyl is polyethylene glycol (PEG). In some embodiments. W² is PEG, wherein the PEG has 1-10 units. In some embodiments, W² is PEG₁₋₈. In some embodiments, W² is PEG₁₋₆. In some embodiments, W² is PEG₁₋₄. In some embodiments, W² is PEG₁₋₃.

In some embodiments of Formula (A), (A-1), or (IV), W² is hydrogen.

In some embodiments of Formula (A), (A-1), or (IV), R^{W} is hydrogen. In some embodiments, R^{W} is optionally substituted C₁-C₂₀ alkyl.

In some embodiments of Formula (A), (A-1), or (IV), W² and R^{W} together with the nitrogen to which they are attached form an optionally substituted 4 to 8-membered heterocycloalkyl which is partially or fully unsaturated. In some embodiments, W² and R^{W} together with the nitrogen to which they are attached form an optionally substituted 4 to 7-membered heterocycloalkyl which is partially or fully unsaturated. In some embodiments, W² and R^{W} together with the nitrogen to which they are attached form an optionally substituted 4-membered heterocycloalkyl. In some embodiments, W² and R^{W} together with the nitrogen to which they are attached form an optionally substituted 5-membered heterocycloalkyl. In some embodiments, W² and R^{W} together with the nitrogen to which they are attached form an optionally substituted 6-membered heterocycloalkyl. In some embodiments, W² and R^{W} together with the nitrogen to which they are attached form an optionally substituted 7-membered heterocycloalkyl.

In some embodiments, the transcription modulator molecule has the structure of Formula (V), or a pharmaceutically acceptable salt thereof:

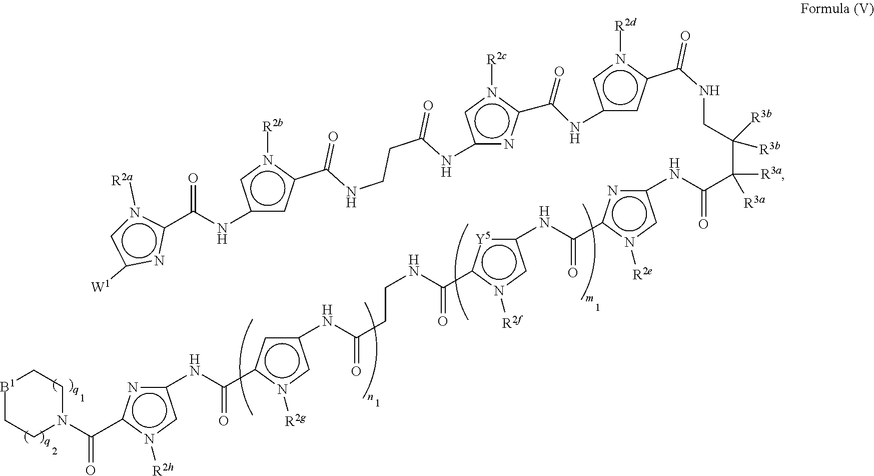

wherein:
B¹ is —CR^{5a}R^{5b}—, —O—, —NR^{5b}—, —S—, —S(O)—, or —S(O)₂—; or B¹ is

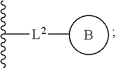

wherein
R^{5a} is hydrogen, —OH, or optionally substituted C₁-C₂₀ alkyl;
R^{5b} is hydrogen, optionally substituted C₁-C₂₀ alkyl, optionally substituted C₂-C₂₀ alkenyl, optionally substituted C₂-C₂₀ alkynyl, optionally substituted C₁-C₂₀ heteroalkyl, —C(O)OR⁶, or —C(O)R⁶; or
R^{5a} and R^{5b} together with the nitrogen atom to which they are attached form an optionally substituted 4 to 8-membered heterocycloalkyl;
R⁶ is hydrogen, optionally substituted C₁-C₂₀ alkyl, optionally substituted C₁-C₁₀ haloalkyl, optionally substituted C₃-C₆ cycloalkyl, optionally substituted 3 to 6-membered heterocycloalkyl, or optionally substituted phenyl;
ring B is absent, optionally substituted C₃-C₆ cycloalkyl, optionally substituted 4 to 6-membered heterocycloalkyl, optionally substituted phenyl, or optionally substituted 5 to 10-membered heteroaryl;
L² is absent, C₁-C₄ alkylene, C₂-C₄ alkynylene, or C₂-C₄ alkynylene;
W¹ is hydrogen or —N═C(N(R^{1e})₂)₂, wherein each R^{1e} is independently hydrogen or C₁-C₃ alkyl;
each Y⁵ is independently N or CH;
each R^{2a}, R^{2b}, R^{2c}, R^{2d}, R^{2e}, R^{2f}, R^{2g}, and R^{2h} is independently hydrogen, optionally substituted C₁-C₅₀ alkyl, optionally substituted C₂-C₅₀ alkenyl, optionally substituted C₂-C₅₀ alkynyl, optionally substituted C₁-C₅₀ heteroalkyl, optionally substituted C₂-C₅₀ heteroalkenyl, optionally substituted C₂-C₅₀ heteroalkynyl, optionally substituted C₁-C₅₀ haloalkyl, optionally substituted C₃-C₅ cycloalkyl, optionally substituted 3 to 8-membered heterocycloalkyl, or optionally substituted PEG₁₋₅₀; each of which is optionally substituted with one or more R¹⁰;
each R^{3a} and R^{3b} is independently hydrogen, halogen, —NR^{11a}R^{11b}, or —NHC(O)R¹², wherein
   R^{11a} and R^{11b} are each independently hydrogen, alkyl, or PEG;
   R¹² is alkyl, PEG, cycloalkyl, heterocycloalkyl, or phenyl;
or two R^{3a} or two R^{3b} together with the carbon atom to which they are attached form a C₃-C₆ cycloalkyl or 4 to 6-membered heterocycloalkyl;
each R¹⁰ is independently —CN, —OH, —OR^{10a}, —N₃, —NR^{10a}R^{10b}, —CO(O)R^{10c}, —C(O)OR^{10c}, —C(O)NR^{10a}R^{10b}, —NHC(O)R^{10c}, —NHC(O)OR^{10c}, —OC(O)NR^{10a}R^{10b}, or optionally substituted 5 to 10-membered heteroaryl; wherein
   R^{10a} and R^{10b} are each independently hydrogen, alkyl, or PEG;
   R^{10c} is alkyl, PEG, cycloalkyl, heterocycloalkyl, or phenyl;
n₁ and m₁ are each independently 0 or 1; and
q₁ and q₂ are each independently 0-2.

In some embodiments of Formula (C), (C-1), (III) or (V), q₁ and q₂ are each 2. In some embodiments, q₁ and q₂ are each 1. In some embodiments, q₁ and q₂ are each 0. In some embodiments, q₁ is 1 or 2; and q₂ is 0. In some embodiments, q₁ is 0; and q₂ is 1 or 2.

In some embodiments of Formula (C), (C-1), (III), or (V), B¹ is —CR^{5a}R^{5b}—, —O—, —NR^{5b}—, or —S—. In some embodiments, B¹ is —O—, —NR^{5b}—, or —S—. In some embodiments, B¹ is —O—. In some embodiments, B¹ is —S—, —S(O)—, or —S(O)₂—. In some embodiments. B¹ is —S—. In some embodiments, B¹ is —S(O)—. In some embodiments, B¹ is —S(O)₂—. In some embodiments, B¹ is —NR^{5b}—. In some embodiments, B¹ is —NH—. In some embodiments, B¹ is —CR^{5a}R^{5b}—. In some embodiments, B¹ is —CH₂—.

In some embodiments of Formula (C), (C-1), (III), or (V), B¹ is

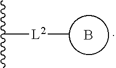

In some embodiment of Formula (C), (C-1), (III), or (V), ring B is an optionally substituted cycloalkyl or optionally substituted heterocycloalkyl. In some embodiments, ring B is an optionally substituted cycloalkyl. In some embodiments, ring B is a C₃-C₈ cycloalkyl. In some embodiments, ring B is a C₃-C₆ cycloalkyl. In some embodiments, ring B is a cyclopropyl, cyclobutyl, cyclopentyl, or cyclohexyl.

In some embodiments of Formula (C), (C-1), (III), or (V), ring B is an optionally substituted 4 to 8-membered heterocycloalkyl. In some embodiments, ring B is an optionally substituted 5 to 7-membered heterocycloalkyl. In some embodiments, ring B is an optionally substituted piperidine or an optionally substituted piperazine. In some embodiments, ring B is an optionally substituted piperidine. In some embodiments, ring B is an optionally substituted piperazine. In some embodiments, ring B is an optionally substituted morpholine.

In some embodiments of Formula (C), (C-1), (C-2), (III), or (V), L² is absent, C₁-C₄ alkylene, C₂-C₄ alkynelene, or C₂-C₄ alkynylene. In some embodiments, L² is C₁-C₄ alkylene. In some embodiments, L² is C₂-C₄ alkynelene. In some embodiments, L² is C₂-C₄ alkynylene.

In some embodiments of Formula (C), (C-1), (C-2), (III), or (V), L² is —CH₂—, —CH₂CH₂—, —C≡C—, or —C≡C—C≡C—. In some embodiments, L² is —CH₂— or —CH₂CH₂—. In some embodiments, L² is —CH₂—. In some embodiments, L² is —CH₂CH₂—. In some embodiments, L² is —C≡C—. In some embodiments, L² is —C≡C—C≡C—.

In some embodiments of Formula (C), (C-1), (C-2), (III), or (V), L² is absent.

In some embodiments of Formula (C), (III), or (V), R^{5a} is an optionally substituted C₁-C₂₀ alkyl. In some embodiments, R^{5a} is hydrogen. In some embodiments, R^{5a} is —OH.

In some embodiments of Formula (C), (III), or (V), R^{5b} is an optionally substituted C₁-C₂₀ alkyl, optionally substituted C₂-C₂₀ alkenyl, or optionally substituted C₂-C₂₀ alkynyl. In some embodiments, R^{5b} is C₁-C₂₀ alkyl. In some embodiments, R^{5b} is C₂-C₂₀ alkenyl. In some embodiments, C₂-C₂₀ alkynyl. In some embodiments, R^{5b} is —C(O)OR⁶ or —C(O)R⁶. In some embodiments, R^{6b} is —C(O)OR⁶. In some embodiments, Rob is —C(O)R⁶. In some embodiments, Rob is hydrogen.

In some embodiments of Formula (C), (III), or (V), R^{5a} and R^{5b} together with the nitrogen atom to which they are attached form an optionally substituted 4 to 8-membered heterocycloalkyl. In some embodiments, R^{5a} and R^{5b} together with the nitrogen atom to which they are attached form an optionally substituted 5 to 7-membered heterocycloalkyl. In some embodiments, R^{5a} and R^{5b} together with the nitrogen atom to which they are attached form an optionally substituted 5-membered heterocycloalkyl. In some embodiments, R^{5a} and R^{5b} together with the nitrogen atom to which they are attached form an optionally substituted 6-membered heterocycloalkyl. In some embodiments, R^{5a} and R^{5b} together with the nitrogen atom to which they are attached form an optionally substituted 7-membered heterocycloalkyl.

In some embodiments of Formula (C), (III), or (V), R⁶ is an optionally substituted C₁-C₂₀ alkyl, optionally substituted C₁-C₁₀ haloalkyl, optionally substituted C₁-C₆ cycloalkyl, or optionally substituted 4 to 6-membered heterocycloalkyl. In some embodiments, R⁶ is an optionally substituted C₁-C₂₀ alkyl. In some embodiments, R⁶ is C₃-C₆ cycloalkyl or 4 to 6-membered heterocycloalkyl. In some embodiments, R⁶ is optionally substituted phenyl.

In another aspect, provided herein is a transcription modulator molecule having a structure of Formula (VI), or a pharmaceutically acceptable salt thereof:

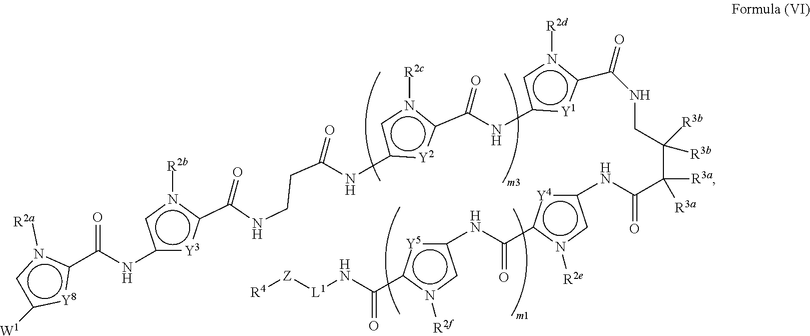

wherein:
W¹ is hydrogen or —N═C(N(R^{1e})₂)₂, wherein each R^{1e} is independently hydrogen or C₁-C₃ alkyl;
each Y¹, Y², Y³, Y⁴, Y⁵, and Y⁸ is independently N or CH;
L¹ is C₁-C₂₀ alkylene or C₂-C₂₀ heteroalkylene;
Z is absent, —C(O)—, or —C(═NH)—;
R⁴ is C₁-C₆ alkyl, —OR^{4b}, or —NR^{4a}R^{4b}; wherein
   R^{4a} is hydrogen, optionally substituted C₁-C₂₀ alkyl, or optionally substituted C₁-C₂₀ heteroalkyl;
   R^{4b} is optionally substituted C₁-C₂₀ alkyl, optionally substituted C₂-C₂₀ alkenyl, optionally substituted C₂-C₂₀ alkynyl, optionally substituted C₁-C₂₀ aminoalkyl, optionally substituted C₁-C₂₀ haloalkyl, optionally substituted C₁-C₂₀ heteroalkyl, optionally substituted C₁-C₂₀ hydroxy alkyl, optionally substituted C₃-C₈ cycloalkyl, optionally substituted 4 to 8-membered heterocycloalkyl, optionally substituted phenyl, or optionally substituted 5 to 10-membered heteroaryl; or
   R^{4a} and R^{4b} together with the nitrogen to which they are attached form an optionally substituted 4 to 8-membered heterocycloalkyl which is partially or fully unsaturated;
each R^{2a}, R^{2b}, R^{2c}, R^{2d}, R^{2e}, and R^{2f} is independently hydrogen, optionally substituted C₁-C₅₀ alkyl, optionally substituted C₂-C₅₀ alkenyl, optionally substituted C₂-C₅₀ alkynyl, optionally substituted C₁-C₅₀ heteroalkyl, optionally substituted C₂-C₅₀ heteroalkenyl, optionally substituted C₂-C₅₀ heteroalkynyl, optionally substituted C₁-C₅₀ haloalkyl, optionally substituted C₃-C₈ cycloalkyl, optionally substituted 3 to 8-membered heterocycloalkyl, or optionally substituted PEG₁₋₅₀; each of which is optionally substituted with one or more R¹⁰;
each R^{3a} and R^{3b} is independently hydrogen, halogen, —NR^{11a}R^{11b}, or —NHC(O)R¹², wherein
   R^{11a} and R^{11b} are each independently hydrogen, alkyl, or PEG;
   R¹² is alkyl, PEG, cycloalkyl, heterocycloalkyl, or phenyl;
or two R^{3a} or two R^{3b} together with the carbon atom to which they are attached form a C₃-C₆ cycloalkyl or 4 to 6-membered heterocycloalkyl;
each R¹⁰ is independently —CN, —OH, —OR^{10a}, —N₃, —NR^{10a}R^{10b}, —CO(O)R^{10c}, —C(O)OR^{10c}, —C(O)NR^{10a}R^{10b}, —NHC(O)R^{10c}, —NHC(O)OR^{10c}, —OC(O)NR^{10a}R^{10b}, or optionally substituted 5 to 10-membered heteroaryl; wherein
   R^{10a} and R^{10b} are each independently hydrogen, alkyl, or PEG;
   R^{10c} is alkyl, PEG, cycloalkyl, heterocycloalkyl, or phenyl;
n₃ is 0 or 1; and
m₁ is 1, 2, or 3.

In some embodiments of Formula (VI), Y⁸ is N and Y³ is CH. In some embodiments, Y⁸ is CH and Y³ is N.

In some embodiments of Formula (VI), Y⁸ is N; Y³ is CH; and Y¹ is CH. In some embodiments, Y⁸ is N; Y³ is CH; and Y¹ is N.

In some embodiments of Formula (VI), L¹ is C₁-C₁₀ alkylene or C₂-C₁₀ heteroalkylene. In some embodiments, L¹ is C₁-C₁₀ alkylene, C₁-C₈ alkylene, C₁-C₆ alkylene, C₁-C₅ alkylene, C₁-C₄ alkylene, C₁-C₃ alkylene, or C₁-C₂ alkylene. In some embodiments, L¹ is C₁-C₄ alkylene. In some embodiments, L¹ is C₁-C₃ alkylene. In some embodiments, L¹ is C₁-C₂ alkylene. In some embodiments, L¹ is C₂-C₁₀ heteroalkylene, C₂-C₈ heteroalkylene, C₂-C₆ heterolkylene, C₂-C₅ heteroalkylene, or C₂-C₄ heteroalkylene. In some embodiments, L¹ is C₂-C₁₀ heteroalkylene. In some embodiments, L¹ is C₂-C₈ heteroalkylene. In some embodiments, L¹ is C₁-C₆ heterolkylene. In some embodiments, L¹ is C₂-C₅ heteroalkylene. In some embodiments, L¹ is C₁-C₄ heteroalkylene.

In some embodiments of Formula (VI), the heteroalkylene is polyethylene glycol. In some embodiments, L¹ is PEG₁₋₁₀. In some embodiments. L¹ is PEG₁₋₈. In some embodiments, L¹ is —(CH₂CH₂—O)_{y1}, wherein y₁ is an integer in the range of 1-10. In some embodiments, y₁ is an integer in the range of 1-8. In some embodiments, y₁ is an integer in the range of 1-6. In some embodiments, y₁ is an integer in the range of 1-4. In some embodiments, y₁ is 1-2. In some embodiments, the heteroalkylene comprises —(CH₂)ₓ₃N(R^{a})(CH₂)ₓ₄—, wherein Ra is hydrogen or an optionally substituted C₁-C₆ alkyl; and each x₃ and x₄ is independently an integer in the range of 1-6.

In some embodiments of Formula (VI), Z is —C(O)—; and R⁴ is —OR^{4b}. In some embodiments, Z is —C(O)—; and R⁴ is —NR^{4a}R^{4b}. In some embodiments, Z is absent; and R⁴ is —OR^{4b}. In some embodiments, Z is absent; and R⁴ is —NR^{4a}R^{4b}. In some embodiments, Z is —C(O)—; and R⁴ is C₁-C₆ alkyl. In some embodiments of Formula (A), (A-1), (I), (Ia), or (Ib), Z is absent; and R⁴ is C₁-C₆ alkyl.

In some embodiments of Formula (VI), n₃ is 1 and m₁ is 1, 2, or 3. In some embodiments, n₃ is 1 and m₁ is 1. In some embodiments, n₃ is 1 and m₁ is 2. In some embodiments, n₃ is 1 and m₁ is 3.

In some embodiments of Formula (VI), n₃ is 0 and m₁ is 1, 2 or 3. In some embodiments, n₃ is 0 and m₁ is 2 or 3. In some embodiments, n₃ is 0 and m₁ is 1. In some embodiments, n₃ is 0 and m₁ is 2. In some embodiments, n₃ is 0 and m₁ is 3.

In some embodiments of Formula (I), (Ia), (Ib), (II), (IIa), (III), (IV), (V), or (VI), W¹ is —N═C(N(R^{1e})₂)₂, wherein each R^{1e} is independently hydrogen or C₁-C₃ alkyl. In some embodiments, each R^{1e} is independently hydrogen. In some embodiments, each R^{1e} is independently C₁-C₃ alkyl.

In some embodiments of Formula (I), (Ia), (Ib), (II), (IIa), (III), (IV), (V), or (VI), W¹ is —N═C(N(CH₃)₂)₂. In some embodiments, W¹ is hydrogen or —N═C(NH₂)₂.

In some embodiments of Formula (I), (Ia), (Ib), (II), (IIa), (III), (IV), (V), or (VI), W¹ is hydrogen.

In some embodiments of Formula (A), (A-1), (I), or (II), each Y¹, Y², Y³, Y⁴, Y⁵, Y⁶, Y⁷, and Y⁸ is independently N. In some embodiments, each Y¹, Y², Y³, Y⁴, Y⁵, Y⁶, Y⁷, and Y⁸ is independently CH.

In some embodiments, each Y², Y⁴, Y⁷, and Y⁸ is independently N; and each Y¹, Y³, and Y⁶ is independently CH. In some embodiments, each Y², Y⁴, Y⁷, and Y⁸ is independently N. In some embodiments, each Y¹, Y³, and Y⁶ is independently CH.

In some embodiments of Formula (A), (A-1), (I), (II), or (VI), each Y¹ is independently N. In some embodiments, each Y¹ is independently CH.

In some embodiments of Formula (A), (A-1), (I), (II), or (VI), each Y² is independently N. In some embodiments, each Y² is independently CH.

In some embodiments of Formula (A), (A-1), (I), (II), or (VI), each Y³ is independently N. In some embodiments, each Y³ is independently CH.

In some embodiments of Formula (A), (A-1), (I), (II), or (VI), each Y⁴ is independently N. In some embodiments, each Y⁴ is independently CH.

In some embodiments of Formula (A), (A-1), (I), (Ia), (Ib), (II), (IIa), (III), (IV), (V), or (VI), each Y⁵ is independently N. In some embodiments, each Y⁵ is independently CH.

In some embodiments of Formula (A), (A-1), (I), or (II), each Y⁶ is independently N. In some embodiments, each Y⁶ is independently CH.

In some embodiments of Formula (A), (A-1), (I), or (II), each Y⁷ is independently N. In some embodiments, each Y⁷ is independently CH.

In some embodiments of Formula (A), (A-1), (I), (II), or (VI), each Y⁸ is independently N. In some embodiments, each Y⁸ is independently CH.

In some embodiments of Formula (A), (A-1), (I), (Ia), (Ib), (III), or (VI), Z is absent or —C(O)—. In some embodiments, Z is absent. In some embodiments, Z is —C(O)—. In some embodiments, Z is —C(═NH)—.

In some embodiments of Formula (I), (Ia), (Ib), (II), (IV), or (V), each R^{2a}, R^{2b}, R^{2c}, R^{2d}, R^{2e}, R^{2f}, R^{2g}, and R^{2h} is independently hydrogen, optionally substituted C₁-C₅₀ alkyl, optionally substituted C₂-C₅₀ alkenyl, optionally substituted C₂-C₂₀ alkynyl, optionally substituted C₁-C₅₀ heteroalkyl, optionally substituted C₂-C₅₀ heteroalkenyl, optionally substituted C₂-C₅₀ heteroalkynyl, optionally substituted C₁-C₅₀ haloalkyl, optionally substituted C₃-C₅ cycloalkyl, optionally substituted 3 to 8-membered heterocycloalkyl, or optionally substituted PEG₁₋₅₀; each of which is optionally substituted with one or more R¹⁰. In some embodiments, each R^{2a}, R^{2b}, R^{2c}, R^{2d}, R^{2e}, R^{2f}, R^{2g}, and R^{2h} is independently hydrogen, optionally substituted C₁-C₂₀ alkyl, optionally substituted C₂-C₂₀ alkenyl, optionally substituted C₂-C₂₀ alkynyl, optionally substituted C₁-C₂₀ heteroalkyl, optionally substituted C₂-C₂₀ heteroalkenyl, optionally substituted C₂-C₂₀ heteroalkynyl, optionally substituted C₁-C₂₀ haloalkyl, optionally substituted C₃-C₅ cycloalkyl, optionally substituted 3 to 8-membered heterocycloalkyl, or optionally substituted PEG₁₋₂₀; each of which is optionally substituted with one or more R¹⁰. In some embodiments, each R^{2a}, R^{2b}, R^{2c}, R^{2d}, R^{2e}, R^{2f}, R^{2g}, and R^{2h} is independently hydrogen, optionally substituted C₁-C₁₀ alkyl, optionally substituted C₂-C₁₀ alkenyl, optionally substituted C₂-C₁₀ alkynyl, optionally substituted C₁-C₁₀ heteroalkyl, optionally substituted C₂-C₁₀ heteroalkenyl, optionally substituted C₂-C₁₀ heteroalkynyl, optionally substituted C₁-C₁₀ haloalkyl, or optionally substituted PEG₁₋₁₀.

In some embodiments of Formula (I), (Ia), (Tb), (II), (IV), or (V), each R^{2a}, R^{2b}, R^{2c}, R^{2d}, R^{2e}, R^{2g}, and R^{2h} is independently hydrogen, optionally substituted C₁-C₁₀ alkyl, optionally substituted C₁-C₁₀ heteroalkyl, optionally substituted C₁-C₁₀ haloalkyl, optionally substituted C₁-C₁₀ alkylamino, or optionally substituted PEG₁₋₁₀. In some embodiments, each R^{2a}, R^{2b}, R^{2c}, R^{2d}, R^{2e}, R^{2g}, and R^{2h} is independently an optionally substituted C₁-C₁₀ heteroalkyl. In some embodiments, each R², R^{2b}, R^{2c}, R^{2d}, R^{2e}, R^{2g}, and R^{2h}, is independently an optionally substituted C₁-C₁₀ haloalkyl. In some embodiments, each R^{2a}, R^{2b}, R^{2c}, R^{2d}, R^{2e}, R^{2g}, and R^{2h} is independently —CF₃ or —CH₂CF₃, or —CH₂CH₂CF₃. In some embodiments, each R^{2a}, R^{2b}, R^{2c}, R^{2d}, R^{2e}, R^{2g}, and R^{2h} is independently an optionally substituted C₁-C₁₀ alkylamino. In some embodiments, each R^{2a}, R^{2b}, R^{2c}, R^{2d}, R^{2e}, R^{2g}, and R^{2h} is independently an optionally substituted PEG₁₋₁₀. In some embodiments, each R^{2a}, R^{2b}, R^{2c}, R^{2d}, R^{2e}, R^{2g}, and R^{2h} is independently an optionally substituted C₁-C₁₀ alkyl. In some embodiments, each R^{2a}, R^{2b}, R^{2c}, R^{2d}, R^{2e}, R^{2g}, and R^{2h} is independently methyl, ethyl, isopropyl, isobutyl, sec-butyl, or tert-butyl. In some embodiments, each R^{2a}, R^{2b}, R^{2c}, R^{2d}, R^{2e}, R^{2f}, R^{2g}, and R^{2g} is independently hydrogen, methyl, ethyl, or isopropyl. In some embodiments, each R^{2a}, R^{2b}, R^{2c}, R^{2e}, R^{2f}, R^{2g}, and R^{2h} is independently isopropyl. In some embodiments, each R^{2a}, R^{2b}, R^{2c}, R^{2d}, R^{2e}, R^{2g}, and R^{2h} is independently ethyl. In some embodiments, each R^{2a}, R^{2b}, R^{2c}, R^{2e}, R^{2f}, R^{2g}, and R^{2h} is methyl. In some embodiments, each R^{2a}, R^{2b}, R^{2c}, R^{2d}, R^{2e}, R^{2f}, R^{2g}, and R^{2h} is independently hydrogen.

In some embodiments of Formula (I), (Ia), (Tb), (II), (IV), or (V), each R^{2a}, R^{2b}, R^{2c}, R^{2d}, R^{2e}, R^{2f}, R^{2g}, and R^{2h} is independently hydrogen, optionally substituted C₁-C₁₀ alkyl, optionally substituted C₁-C₁₀ heteroalkyl, optionally substituted C₁-C₁₀ haloalkyl, or optionally substituted PEG₁₋₁₀; each of which is optionally substituted with one or more R¹⁰. In some embodiments, each R^{2a}, R^{2b}, R^{2c}, R^{2d}, R^{2e}, R^{2f}, R^{2g}, and R^{2h} is independently C₁-C₁₀ alkyl, each of which is optionally substituted with one or more R¹⁰.

In some embodiments of Formula (I), (Ia), (Tb), (II), (IV), or (V), each R^{2a}, R^{2b}, R^{2d}, and R^{2g} is independently an optionally substituted C₁-C₁₀ alkyl, optionally substituted C₁-C₁₀ heteroalkyl, optionally substituted C₁-C₁₀ haloalkyl, or optionally substituted PEG₁₋₁₀; each of which is optionally substituted with one or more R¹⁰; and each of R^{2c}, R^{2e}, and R^{2f}, is independently unsubstituted C₁-C₁₀ alkyl.

In some embodiments of Formula (I), (Ia), (Ib), (II), (IV), or (V), each of R^{2c}, R^{2e}, and R^{2h} is independently unsubstituted C₁-C₁₀ alkyl. In some embodiments, each of R^{2c}, R^{2e}, and R^{2h} is independently methyl, ethyl, isopropyl, or tert-butyl. In some embodiments, each of R^{2c}, R^{2e}, and R^{2h} is independently methyl, ethyl, or isopropyl. In some embodiments, each of R^{2c}, R^{2e}, and R^{2h} is independently methyl or isopropyl. In some embodiments, each of R^{2c}, R^{2e}, and R^{2h} is methyl.

In some embodiments of Formula (I), (Ia), (Ib), (II), (IV), or (V), each R^{2a}, R^{2b}, and R^{2g} is independently unsubstituted C₁-C₁₀ alkyl. In some embodiments, each R^{2a}, R^{2b}, and R^{2g} is independently methyl, ethyl, isopropyl, or tert-butyl. In some embodiments, each R^{2a}, R^{2b}, and R^{2g} is independently methyl, ethyl, or isopropyl. In some embodiments, each R^{2a}, R^{2b}, and R^{2g} is independently methyl or isopropyl. In some embodiments, each R^{2a}, R^{2b}, and R^{2g} is methyl.

In some embodiments of Formula (I), (Ia), (Ib), (II), (IV), or (V), each R^{2a}, R^{2b}, R^{2c}, R^{2e}, R^{2f}, R^{2g}, and R^{2h} is independently unsubstituted alkyl C₁-C₁₀ alkyl; and R^{2d} is C₁-C₁₀ alkyl, which is optionally substituted with one or more R¹⁰. In some embodiments, each R^{2a}, R^{2b}, R^{2c}, R^{2e}, R^{2f}, R^{2g}, and R^{2h} is methyl; and R^{2a} is C₁-C₁₀ alkyl, which is optionally substituted with one or more R¹⁰.

In some embodiments of Formula (I), (Ia), (Ib), (II), (IV), (V), or (VI), R^{2a} is hydrogen, an optionally substituted C₁-C₁₀ alkyl, optionally substituted C₁-C₁₀ heteroalkyl, optionally substituted C₁-C₁₀ haloalkyl, or optionally substituted PEG₁₋₁₀, each of which is optionally substituted with one or more R¹⁰. In some embodiments, R^{2a} is an optionally substituted C₁-C₁₀ alkyl, optionally substituted C₁-C₁₀ heteroalkyl, or optionally substituted C₁-C₁₀ haloalkyl. In some embodiments, R^{2a} is an optionally substituted C₁-C₁₀ alkyl which is substituted with one or more R¹⁰. In some embodiments, R^{2a} is unsubstituted C₁-C₁₀ alkyl. In some embodiments, R^{2a} is methyl, ethyl, or isopropyl. In some embodiments, R² is isopropyl. In some embodiments, R^{2a} is ethyl. In some embodiments, R² is methyl. In some embodiments, R^{2a} is hydrogen.

In some embodiments of Formula (I), (Ia), (Ib), (II), (IV), (V), or (VI), R^{2b} is hydrogen, an optionally substituted C₁-C₁₀ alkyl, optionally substituted C₁-C₁₀ heteroalkyl, optionally substituted C₁-C₁₀ haloalkyl, or optionally substituted PEG₁₋₁₀, each of which is optionally substituted with one or more R¹⁰. In some embodiments, R^{2b} is an optionally substituted C₁-C₁₀ alkyl, optionally substituted C₁-C₁₀ heteroalkyl, or optionally substituted C₁-C₁₀ haloalkyl. In some embodiments, R^{2b} is an optionally substituted C₁-C₁₀ alkyl which is substituted with one or more R¹⁰. In some embodiments, R^{2b} is unsubstituted C₁-C₁₀ alkyl. In some embodiments, R^{2b} is methyl, ethyl, or isopropyl. In some embodiments, R^{2b} is isopropyl. In some embodiments, R^{2b} is ethyl. In some embodiments, R^{2b} is methyl. In some embodiments, R^{2b} is hydrogen.

In some embodiments of Formula (I), (Ia), (Ib), (II), (IV), (V), or (VI), each R², is independently hydrogen, an optionally substituted C₁-C₁₀ alkyl, optionally substituted C₁-C₁₀ heteroalkyl, optionally substituted C₁-C₁ haloalkyl, or optionally substituted PEG₁₋₁₀, each of which is optionally substituted with one or more R¹⁰. In some embodiments, each R^{2c} is independently an optionally substituted C₁-C₁₀ alkyl, optionally substituted C₁-C₁₀ heteroalkyl, or optionally substituted C₁-C₁₀ haloalkyl. In some embodiments, each R^{2c} is independently an optionally substituted C₁-C₁₀ alkyl which is substituted with one or more R¹⁰. In some embodiments, each R^{2c} is independently an unsubstituted C₁-C₁₀ alkyl. In some embodiments, each R^{2c}, is independently methyl, ethyl, or isopropyl. In some embodiments, each R^{2c} is independently isopropyl. In some embodiments, each R^{2c} is independently ethyl. In some embodiments, each R^{2c} is independently methyl. In some embodiments, each R^{2c} is independently hydrogen.

In some embodiments of Formula (I), (Ia), (Ib), (II), (IIa), (III), (IV), (V), or (VI), R^{2d} is hydrogen, an optionally substituted C₁-C₁₀ alkyl, optionally substituted C₁-C₁₀ heteroalkyl, optionally substituted C₁-C₁₀ haloalkyl, or optionally substituted PEG₁₋₁₀, each of which is optionally substituted with one or more R¹⁰. In some embodiments, R^{2d} is an optionally substituted C₁-C₁₀ alkyl, optionally substituted C₁-C₁₀ heteroalkyl, or optionally substituted C₁-C₁₀ haloalkyl. In some embodiments, R^{2a} is an optionally substituted C₁-C₁₀ alkyl which is substituted with one or more R¹⁰. In some embodiments, R^{2d} is unsubstituted C₁-C₁₀ alkyl. In some embodiments, R^{2d} is methyl, ethyl, or isopropyl. In some embodiments, R^{2d} is isopropyl. In some embodiments, R^{2d} is ethyl. In some embodiments, R^{2d} is methyl. In some embodiments, R^{2d} is hydrogen.

In some embodiments of Formula (I), (Ia), (Ib), (II), (IV), (V), or (VI), R^{2e} is hydrogen, an optionally substituted C₁-C₁₀ alkyl, optionally substituted C₁-C₁₀ heteroalkyl, optionally substituted C₁-C₁₀ haloalkyl, or optionally substituted PEG₁₋₁₀, each of which is optionally substituted with one or more R¹⁰. In some embodiments, R^{2e} is an optionally substituted C₁-C₁₀ alkyl, optionally substituted C₁-C₁₀ heteroalkyl, or optionally substituted C₁-C₁₀ haloalkyl. In some embodiments, R^{2e} is an optionally substituted C₁-C₁₀ alkyl which is substituted with one or more R¹⁰. In some embodiments, R^{2e} is unsubstituted C₁-C₁₀ alkyl. In some embodiments, R^{2e} is methyl, ethyl, or isopropyl. In some embodiments, R^{2e} is isopropyl. In some embodiments, R^{2e} is ethyl. In some embodiments, R^{2e} is methyl. In some embodiments, R^{2e} is hydrogen.

In some embodiments of Formula (I), (Ia), (Ib), (II), (IV), (V), or (VI), each R^{2f} is independently hydrogen, an optionally substituted C₁-C₁₀ alkyl, optionally substituted C₁-C₁₀ heteroalkyl, optionally substituted C₁-C₁₀ haloalkyl, or optionally substituted PEG₁₋₁₀, each of which is optionally substituted with one or more R¹⁰. In some embodiments, each R^{2f} is independently an optionally substituted C₁-C₁₀ alkyl, optionally substituted C₁-C₁₀ heteroalkyl, or optionally substituted C₁-C₁₀ haloalkyl. In some embodiments, each R^{2f} is independently an optionally substituted C₁-C₁₀ alkyl which is substituted with one or more R¹⁰. In some embodiments, each R^{2f} is independently an unsubstituted C₁-C₁₀ alkyl. In some embodiments, each R^{2d} is independently methyl, ethyl, or isopropyl. In some embodiments, each R^{2f} is independently isopropyl. In some embodiments, each R^{2f} is independently ethyl. In some embodiments, each R^{2f} is independently methyl. In some embodiments, each R^{2f} is independently hydrogen.

In some embodiments of Formula (I), (Ia), (Ib), (II), (IV), or (V), R^{2g} is hydrogen, an optionally substituted C₁-C₁₀ alkyl, optionally substituted C₁-C₁₀ heteroalkyl, optionally substituted C₁-C₁₀ haloalkyl, or optionally substituted PEG₁₋₁₀. each of which is optionally substituted with one or more R¹⁰. In some embodiments, R²⁹ is an optionally substituted C₁-C₁₀ alkyl, optionally substituted C₁-C₁₀ heteroalkyl, or optionally substituted C₁-C₁₀ haloalkyl. In some embodiments, R^{2g} is an optionally substituted C₁-C₁₀ alkyl which is substituted with one or more R¹⁰. In some embodiments, R²⁹ is unsubstituted C₁-C₁₀ alkyl. In some embodiments, R^{2g} is methyl, ethyl, or isopropyl. In some embodiments, R^{2g} is isopropyl. In some embodiments, R^{2g} is ethyl. In some embodiments, R^{2g} is methyl. In some embodiments, R^{2g} is hydrogen.

In some embodiments of Formula (I), (Ia), (Ib), (II), (IV), or (V), R^{2h} is hydrogen, an optionally substituted C₁-C₁₀ alkyl, optionally substituted C₁-C₁₀ heteroalkyl, optionally substituted C₁-C₁₀ haloalkyl, or optionally substituted PEG₁₋₁₀. each of which is optionally substituted with one or more R¹⁰. In some embodiments, R^{2f} is an optionally substituted C₁-C₁₀ alkyl, optionally substituted C₁-C₁₀ heteroalkyl, or optionally substituted C₁-C₁₀ haloalkyl. In some embodiments, R^{2f} is an optionally substituted C₁-C₁₀ alkyl which is substituted with one or more R¹⁰. In some embodiments, R^{2h} is unsubstituted C₁-C₁₀ alkyl. In some embodiments, R^{2h} is methyl, ethyl, or isopropyl. In some embodiments, R^{2h} is isopropyl. In some embodiments, R^{2h} is ethyl. In some embodiments, R^{2h} is methyl. In some embodiments, R^{2h} is hydrogen.

In some embodiments of Formula (I), (Ia), (Ib), (II), (IIa), (III), (IV), (V), or (VI), each R^{3a} is independently hydrogen, deuterium, halogen, amino, optionally substituted C₁-C₂₀ alkyl, optionally substituted C₁-C₂₀ haloalkyl, optionally substituted C₁-C₂₀ alkylamino, or optionally substituted C₁-C₂₀ hydroxyalkyl. In some embodiments, each R^{3a} is independently hydrogen, amino, or optionally substituted C₁-C₂₀ alkylamino. In some embodiments, each R^{3a} is hydrogen.

In some embodiments of Formula (I), (Ia), (Ib), (II), (IV), (V), or (VI), each R^{3b} is independently hydrogen, deuterium, halogen, amino, optionally substituted C₁-C₂₀ alkyl, optionally substituted C₁-C₂₀ haloalkyl, optionally substituted C₁-C₂₀ alkylamino, or optionally substituted C₁-C₂₀ hydroxyalkyl. In some embodiments, each R^{3b} is independently hydrogen, amino, or optionally substituted C₁-C₂₀ alkylamino. In some embodiments, each R^{3b} is hydrogen.

In some embodiments of Formula (I), (Ia), (Ib), (II), (IV), (V), or (VI), each R^{3b} is hydrogen; and each R^{3a} is independently hydrogen, halogen, amino, optionally substituted C₁-C₁₀ alkyl, optionally substituted C₁-C₁₀ haloalkyl, optionally substituted C₁-C₁₀ alkylamino, or optionally substituted C₁-C₁₀ hydroxyalkyl. In some embodiments, each R^{3b} is hydrogen; and each R^{3a} is independently hydrogen, halogen, amino, optionally substituted C₁-C₁₀ alkyl, optionally substituted C₁-C₁₀ haloalkyl, optionally substituted C₁-C₁₀ alkylamino, or optionally substituted C₁-C₁₀ hydroxyalkyl. In some embodiments, each R^{3b} is hydrogen; and each R^{3a} is independently hydrogen or amino.

In some embodiments of Formula (I), (Ia), (Ib), (II), (IV), (V), or (VI), each R^{3a} and each R^{3b} is hydrogen.

In some embodiments of Formula (I), (Ia), (Ib), (II), (IV), (V), or (VI), two R^{3a} together with the carbon atom to which they are attached form a C₃-C₆ cycloalkyl. In some embodiments, two R^{3a} together with the carbon atom to which they are attached form a cyclopropyl, cyclobutyl, or cyclopentyl. In some embodiments, two R^{3a} together with the carbon atom to which they are attached form a cyclopropyl. In some embodiments, two R^{3a} together with the carbon atom to which they are attached form a cyclobutyl. In some embodiments, two R^{3a} together with the carbon atom to which they are attached form a cyclopentyl. In some embodiments, two R^{3a} together with the carbon atom to which they are attached form a 4 to 6-membered heterocycloalkyl. In some embodiments, two R^{3a} together with the carbon atom to which they are attached form a 4-membered heterocycloalkyl. In some embodiments, two R^{3a} together with the carbon atom to which they are attached form a 5-membered heterocycloalkyl. In some embodiments, two R^{3a} together with the carbon atom to which they are attached form a 6-membered heterocycloalkyl.

In some embodiments of Formula (I), (Ia), (Ib), (II), (IV), (V), or (VI), two R^{3b} together with the carbon atom to which they are attached form a C₃-C₆ cycloalkyl ring. In some embodiments, two R^{3b} together with the carbon atom to which they are attached form a cyclopropyl, cyclobutyl, or cyclopentyl. In some embodiments, two R^{3b} together with the carbon atom to which they are attached form a cyclopropyl. In some embodiments, two R^{3b} together with the carbon atom to which they are attached form a cyclobutyl. In some embodiments, two R^{3b} together with the carbon atom to which they are attached form a cyclopentyl. In some embodiments, two R^{3b} together with the carbon atom to which they are attached form a 4 to 6-membered heterocycloalkyl. In some embodiments, two R^{3b} together with the carbon atom to which they are attached form a 4-membered heterocycloalkyl. In some embodiments, two R^{3b} together with the carbon atom to which they are attached form a 5-membered heterocycloalkyl. In some embodiments, two R^{3b} together with the carbon atom to which they are attached form a 6-membered heterocycloalkyl.

In some embodiments of Formula (A), (A-1), (I), (Ia), (Ib), or (VI), R^{4a} is hydrogen, optionally substituted C₁-C₂₀ alkyl, or optionally substituted C₁-C₂₀ heteroalkyl. In some embodiments, R^{4a} is an optionally substituted C₁-C₂₀ alkyl or optionally substituted C₁-C₂₀ heteroalkyl. In some embodiments. R^{4a} is an optionally substituted C₁-C₂₀ alkyl. In some embodiments, R^{4a} is an optionally substituted C₁-C₁₅ alkyl. In some embodiments, R^{4a} is an optionally substituted C₁-C₁₀ alkyl. In some embodiments, R^{4a} is an optionally substituted C₁-C₂₀ heteroalkyl. In some embodiments, the heteroalkyl is polyethylene glycol (PEG). In some embodiments, R^{4a} is optionally substituted PEG₁₋₂₀. In some embodiments, R^{4a} is an optionally substituted PEG₁₋₁₅. In some embodiments, R^{4a} is optionally substituted PEG₁₋₁₀. In some embodiments, R^{4a} is hydrogen.

In some embodiments of Formula (A), (A-1), (I), (Ia), (Ib), or (VI), R^{4b} is optionally substituted C₁-C₂₀ alkyl, optionally substituted C₁-C₂₀ aminoalkyl, optionally substituted C₁-C₂₀ haloalkyl optionally substituted C₁-C₂₀ heteroalkyl, or optionally substituted C₁-C₂₀ hydroxyalkyl. In some embodiments, R^{4b} is optionally substituted C₁-C₂₀ alkyl or optionally substituted C₁-C₂₀ heteroalkyl. In some embodiments. R^{4b} is optionally substituted C₁-C₂₀ alkyl. In some embodiments, R^{4b} is optionally substituted C₁-C₁₅ alkyl. In some embodiments, R^{4b} is optionally substituted C₁-C₁₀ alkyl. In some embodiments, R^{4b} is optionally substituted C₁-C₂₀ heteroalkyl. In some embodiments, R^{4b} is optionally substituted C₁-C₁₅ heteroalkyl. In some embodiments, R^{4b} is optionally substituted C₁-C₁₀ heteroalkyl. In some embodiments, the heteroalkyl is polyethylene glycol (PEG). In some embodiments, R^{4b} is PEG₁₋₂₀. In some embodiments, R^{4b} is PEG₁₋₁₄. In some embodiments, R^{4b} is PEG₁₋₁₀.

In some embodiments of Formula (A), (A-1), (I), (Ia), (Ib), or (VI), R^{4b} is optionally substituted C₃-C₈ cycloalkyl, optionally substituted 4 to 8-membered heterocycloalkyl, optionally substituted phenyl, or optionally substituted 5 to 10-membered heteroaryl. In some embodiments, R^{4b} is optionally substituted C₃-C₈ cycloalkyl or optionally substituted 4 to 8-membered heterocycloalkyl. In some embodiments, R^{4b} is optionally substituted C₃-C₆ cycloalkyl or optionally substituted 4 to 6-membered heterocycloalkyl. In some embodiments, R^{4b} is optionally substituted C₃-C₆ cycloalkyl. In some embodiments, R^{4b} is cyclopentyl or cyclohexyl. In some embodiments, R^{4b} is optionally substituted 4 to 6-membered heterocycloalkyl. In some embodiments, R^{4b} is a 5 or 6-membered heterocycloalkyl. In some embodiments, R^{4b} is a piperidine, piperazine, or morpholine. In some embodiments, R^{4b} is a piperidine or piperazine. In some embodiments, R^{4b} is piperidine. In some embodiments, R^{4b} is piperazine.

In some embodiments of Formula (A), (A-1), (I), (Ia), (Ib), or (VI), R^{4a} and R^{4b} together with the nitrogen to which they are attached form an optionally substituted 4 to 8-membered heterocycloalkyl which is partially or fully unsaturated. In some embodiments, R^{4a} and R^{4b} together with the nitrogen to which they are attached form an optionally substituted 4 to 6-membered heterocycloalkyl. In some embodiments, R^{4a} and R^{4b} together with the nitrogen to which they are attached form an optionally substituted 4-membered heterocycloalkyl. In some embodiments, R^{4a} and R^{4b} together with the nitrogen to which they are attached form an optionally substituted 5-membered heterocycloalkyl. In some embodiments, R^{4a} and R^{1b} together with the nitrogen to which they are attached form an optionally substituted 6-membered heterocycloalkyl.

In some embodiments of Formula (I), (Ia), (Ib), (II), (IIa), (III), (IV), (V), or (VI), each R¹⁰ is independently —CN, —OH, —OR^{10a}, —N₃, —NR^{10a}R^{10b}, —CO(O)R^{10a}, —C(O)OR^{10b}, —C(O)NR^{10a}R^{10b}, —NHC(O)R^{10c}, —NHC(O)OR^{10c}, —OC(O)NR^{10a}R^{10b}, or optionally substituted 5 to 10-membered heteroaryl. In some embodiments, each R¹⁰ is independently —CN, —OH, —OR^{10a}—N₃, —NR^{10a}R^{10b}, —C(O)OR^{10c}, —C(O)NR^{10a}R^{10b}, —NHC(O)R^{10c}, or optionally substituted 5-membered heteroaryl. In some embodiments, each R¹⁰ is independently —CN, —OH, —OR^{10a}, —N₃, or —NR^{10a}R^{10b}. In some embodiments, each R¹⁰ is independently —CO(O)R^{10c}, —C(O)OR^{10c}, —C(O)NR^{10a}R^{10b}, —NHC(O)R^{10c}, —NHC(O)OR^{10c}, or —OC(O)NR^{10a}R^{10b}. In some embodiments, each R¹⁰ is independently —C(O)NR^{10a}R^{10b}, —NHC(O)R^{10c}, or —OC(O)NR^{10a}R^{10b}. In some embodiments, each R¹⁰ is independently an optionally substituted 5 to 10-membered heteroaryl. In some embodiments, each R¹⁰ is independently an optionally substituted 5-membered heteroaryl. In some embodiments, each R¹⁰ is independently an optionally substituted triazine.

In some embodiments of Formula (I), (Ia), (Ib), (II), (IIa), (III), (IV), (V), or (VI), each R^{10a} and R^{10b} is independently hydrogen, alkyl, or PEG. In some embodiments, each R^{10a} and R^{10b} is independently hydrogen, C₁-C₂₀ alkyl, or PEG₁₋₂₀. In some embodiments, each R^{10a} and R^{10b} is independently C₁-C₂₀ alkyl. In some embodiments, each R^{10a} and R^{10b} is independently PEG₁₋₂₀. In some embodiments, each R^{10a} and R^{10b} is independently hydrogen.

In some embodiments of Formula (I), (Ia), (Ib), (II), (IIa), (III), (IV), (V), or (VI), R^{10c} is alkyl, PEG, cycloalkyl, heterocycloalkyl, or phenyl. In some embodiments, R^{10c} is C₁-C₂₀ alkyl, PEG₁₋₂₀, C₃-C₆ cycloalkyl, 4 to 6-membered heterocycloalkyl, or phenyl. In some embodiments, R^{10c} is C₁-C₂₀ alkyl or PEG₁₋₂₀. In some embodiments, R^{10c} is C₁-C₂₀ alkyl. In some embodiments, R^{10c} is PEG₁₋₂₀. In some embodiments, R^{10c} is C₃-C₆ cycloalkyl, 4 to 6-membered heterocycloalkyl, or phenyl. In some embodiments, R^{10c} is C₃-C₆ cycloalkyl. In some embodiments, R^{10c} is 4 to 6-membered heterocycloalkyl. In some embodiments, R^{10c} is phenyl.

In some embodiments of Formula (I), (Ia), (Ib), (II), (IIa), (III), (IV), (V), or (VI), R^{11a} and R^{11b} are each independently hydrogen, alkyl, or PEG. In some embodiments, R^{11a} and R^{11b} are each independently hydrogen, C₁-C₂₀alkyl, or PEG₁₋₂₀. In some embodiments, R^{11a} and R^{11b} are each independently C₁-C₂₀alkyl. In some embodiments, R^{11a} and R^{11b} are each independently PEG₁₋₂₀. In some embodiments, R^{11a} and R^{11b} are each independently hydrogen.

In some embodiments of Formula (I), (Ia), (Ib), (II), (IIa), (III), (IV), (V), or (VI), R¹² is alkyl. PEG, cycloalkyl, heterocycloalkyl, or phenyl. In some embodiments, R¹² is C₁-C₂₀ alkyl, PEG₁₋₂₀, C₃-C₆ cycloalkyl, 4 to 6-membered heterocycloalkyl, or phenyl. In some embodiments, R¹² is C₁-C₂₀ alkyl or PEG₁₋₂₀. In some embodiments, R¹² is C₃-C₆ cycloalkyl, 4 to 6-membered heterocycloalkyl, or phenyl. In some embodiments, R¹² is C₁-C₂₀ alkyl. In some embodiments, R¹² is PEG₁₋₂₀. In some embodiments, R¹² is C₃-C₆ cycloalkyl. In some embodiments, R¹² is 4 to 6-membered heterocycloalkyl. In some embodiments, R¹² is phenyl.

In some embodiments of Formula (A), (A-1), (II), or (IIa), each AA is independently a naturally occurring amino acid. In some embodiments, each AA is independently selected from lysine, arginine, serine, threonine, or cysteine. In some embodiments, each AA is independently selected from lysine or arginine. In some embodiments, each AA is independently selected from lysine. In some embodiments, each AA is independently selected from arginine.

In some embodiments of Formula (A), (A-1), (I), (Ia), (Ib), (II), (III), (IV), (V), n₁ is 1. In some embodiments, n₁ is 0.

In some embodiments of Formula (A), (A-1), (I), (Ia), (Ib), (II), (IIa), (III), (IV), (V), or (VI), m₁ is 1, 2, or 3. In some embodiments, m₁ is 1 or 2. In some embodiments, m₁ is 0 or 1. In some embodiments, m₁ is 3. In some embodiments, m₁ is 2. In some embodiments, m₁ is 1. In some embodiments, m₁ is 0.

### Polyamide—DNA Binding Moiety

The binding affinity between the compound and the target gene can be adjusted based on the composition of the polyamide sequence portion of the compound. In some embodiments, the compound is capable of binding the DNA with an affinity of less than about 600 nM, about 500 nM, about 400 nM, about 300 nM, about 250 nM, about 200 nM, about 150 nM, about 100 nM, or about 50 nM. In some embodiments, the compound is capable of binding the DNA with an affinity in the range of about 1-600 nM, 10-500 nM, 20-500 nM, 50-400 nM, or 100-300 nM.

In some embodiments, the compound is capable of binding the DNA with an affinity of less than 500 nM. In some embodiments, the compound is capable of binding the DNA with an affinity of less than about 300 nM. In some embodiments, the compound is capable of binding the DNA with an affinity of less than about 200 nM

The binding affinity between the compound and the target DNA can be determined using a quantitative footprint titration experiment. The experiment involves measuring the dissociation constant Ka of the polyamide for the target sequence at either 24° C., or 37° C., and using either standard polyamide assay solution conditions or approximate intracellular solution conditions.

The compound has a high binding affinity to a sequence having multiple nucleotide repeats comprising CAG and binds to the target nucleotide repeats preferentially over other nucleotide repeats or other nucleotide sequences. In some embodiments, the compound has a higher binding affinity to a sequence having multiple nucleotide repeats comprising CAG than to a sequence having repeats of CGG. In some embodiments, the compound has a higher binding affinity to a sequence having multiple nucleotide repeats comprising CAG than to a sequence having repeats of CCG. In some embodiments, the compound has a higher binding affinity to a sequence having multiple nucleotide repeats comprising CAG than to a sequence having repeats of CCTG. In some embodiments, the compound has a higher binding affinity to a sequence having multiple nucleotide repeats comprising CAG than to a sequence having repeats of TGGAA. In some embodiments, the compound has a higher binding affinity to a sequence having multiple nucleotide repeats comprising CAG than to a sequence having repeats of GGGGCC. In some embodiments, the compound has a higher binding affinity to a sequence having multiple nucleotide repeats comprising CAG than to a sequence having repeats of GAA.

Due to the preferential binding between the polyamide sequence and the target nucleotide repeat, the transcription modulation molecules described herein become localized around regions having multiple nucleotide repeats comprising CAG. In some embodiments, the local concentration of the molecule is higher near a sequence having multiple nucleotide repeats comprising CAG than near a sequence having repeats of CGG. In some embodiments, the local concentration of molecule is higher near a sequence having multiple nucleotide repeats comprising CAG than near a sequence having repeats of CCG. In some embodiments, the local concentration of the molecules is higher near a sequence having multiple nucleotide repeats comprising CAG than near a sequence having repeats of CCTG. In some embodiments, the local concentration of the molecules is higher near a sequence having multiple nucleotide repeats comprising CAG than near a sequence having repeats of TGGAA. In some embodiments, the local concentration of the molecules is higher near a sequence having multiple nucleotide repeats comprising CAG than near a sequence having repeats of GGGGCC. In some embodiments, the local concentration of the molecules is higher near a sequence having multiple nucleotide repeats comprising CAG than near a sequence having repeats of GAA.

In an aspect provided herein, the molecules of the present disclosure preferentially bind to the repeated CAG of atxn1, atxn2, atxn3, cacna1a, atxn7, ttbk2, ppp2r2b, tbp, htt, jph3, ar, or atn1 than to CAG elsewhere in the subject's DNA, due to the high number of CAG repeats associated with atxn1, atxn2, atxn3, cacna1a, atxn7. ttbk2, ppp2r2b, tbp, htt, jph3, ar, or atn1. In some embodiments, the molecules of the present disclosure are more likely to bind to the repeated CAG of atxn1, atxn2, atxn3, cacna1a, atxn7, ppp2r2b, tbp, htt, jph3, ar, or atn1 than to CAG elsewhere in the subject's DNA, due to the high number of CAG repeats associated with atxn1, atxn2, atxn3, cacna1a, atxn7, ppp2r2b, tbp, htt, jph3, ar, or atn1. In some embodiments, the molecules of the present disclosure are more likely to bind to the repeated CAG of 7TBK2 gene than to CAG elsewhere in the subject's DNA, due to the high number of CAG repeats associated with TTBK2.

The polyamide is localized to a sequence having multiple nucleotide repeats comprising CAG and binds to the target nucleotide repeats preferentially over other nucleotide repeats. In some embodiments, the sequence has at least 2, 3, 4, 5, 8, 10, 12, 15, 20, 25, 30, 40, 50, 100, 200, 300, 400, or 500 repeats of CAG. In some embodiments, the sequence comprises at least 1000 nucleotide repeats of CAG. In some embodiments, the sequence comprises at least 500 nucleotide repeats of CAG. In some embodiments, the sequence comprises at least 200 nucleotide repeats of CAG. In some embodiments, the sequence comprises at least 100 nucleotide repeats of CAG. In some embodiments, the sequence comprises at least 50 nucleotide repeats of CAG. In some embodiments, the sequence comprises at least 20 nucleotide repeats of CAG.

The polyamide composed of a pre-selected combination of subunits can selectively bind to the DNA in the minor groove. In their hairpin structure, antiparallel side-by-side pairings of two aromatic amino acids bind to DNA sequences, with a polyamide ring packed specifically against each DNA base. N-Methylpyrrole (Py) favors T, A, and C bases, excluding G; N-methylimidazole (Tm) is a G-reader; and 3-hydroxyl-N-methylpyrrol (Hp) is specific for thymine base. The nucleotide base pairs can be recognized using different pairings of die amino acid subunits using the pairing principle shown in Table A and 1B below. For example, an Im/Py pairing reads G·C by symmetry, a Py/Im pairing reads C·G, an Hp/Py pairing can distinguish T·A from A·T, G·C, and C·G, and a Py/Py pairing nonspecifically discriminates both A·T and T·A from G·C and C·G.

In some embodiments, the polyamide compound comprises Im corresponding to the nucleotide G; Tm or Nt corresponding to the nucleotide pair G; Py corresponding to the nucleotide C, wherein Im is N-alkyl imidazole, Py is N-alkyl pyrrole, Hp is 3-hydroxy N-methyl pyrrole, and β-alanine. In some embodiments, the polyamide comprises Im/Py to correspond to the nucleotide pair G/C, Py/Im to correspond to the nucleotide pair CIG, and wherein Tm is N-alkyl imidazole (e.g., N-methyl imidazole), Py is N-alkyl pyrrole (e.g., N-methyl pyrrole), and Hp is 3-hydroxy N-methyl pyrrole.

TABLE 1A
Base pairing for single amino acid subunit (Favored (+), disfavored (-)).

| Subunit | | G | C | A | T |
|---|---|---|---|---|---|
| Py | | — | + | + | + |
| Im | | + | — | — | |
| (Hp) | 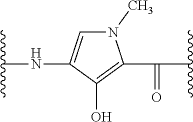 [SMILES: *Nc1cn(C)c(C(*)=O)c1O] | — | — | — | + |
| (Th) | 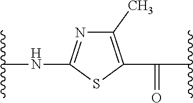 [SMILES: *Nc1nc(C)c(C(*)=O)s1] | — | — | + | + |
| (Pz) | 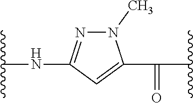 [SMILES: *Nc1cc(C(*)=O)n(C)n1] | — | — | + | + |
| (Tp) | 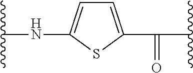 [SMILES: *Nc1ccc(C(*)=O)s1] | — | — | + | + |
| (Nt) | 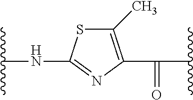 [SMILES: *Nc1nc(C(*)=O)c(C)s1] | + | — | — | — |
| (Ht) | 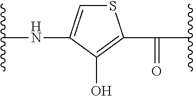 [SMILES: *Nc1csc(C(*)=O)c1O] | — | — | — | + |
| (iPTA) | 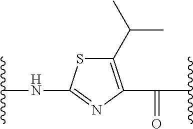 [SMILES: *Nc1nc(C(*)=O)c(C(C)C)s1] | + | — | — | — |
| (CTh) | 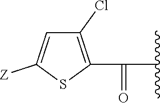 | — | — | — | + |
| (PEG) | 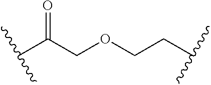 [SMILES: *CCOCC(*)=O] | — | + | + | + |
| (iIm) | 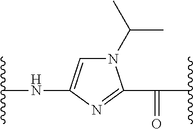 [SMILES: *Nc1cn(C(C)C)c(C(*)=O)n1] | + | — | — | — |
| (Ip) | 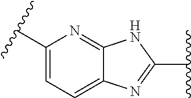 [SMILES: *c1ccc2nc(*)[nH]c2n1] | + | — | — | — |
| (Hz) | 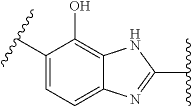 [SMILES: *c1nc2ccc(*)c(O)c2[nH]1] | — | — | — | + |
| (Bi) | 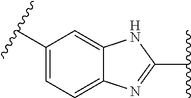 [SMILES: *c1ccc2nc(*)[nH]c2c1] | — | — | — | + |
| (Gly) | 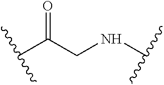 [SMILES: *NCC(*)=O] | — | — | — | — |
| (Beta or β) | 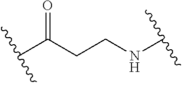 [SMILES: *NCCC(*)=O] | — | — | + | + |
| (gAB) | 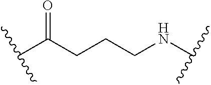 [SMILES: *NCCCC(*)=O] | — | — | + (as a part of the turn) | + (as a part of the turn) |
| (Alx) | 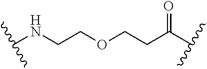 [SMILES: *NCCOCCC(*)=O] | — | + | — | — |
| (Da) | 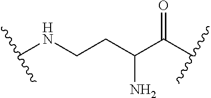 [SMILES: *NCCC(N)C(*)=O] | — | — | + | + |
| (Dp) | 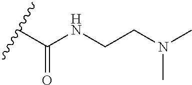 [SMILES: *C(=O)NCCN(C)C] | — | — | + | + |
| (iPP) | 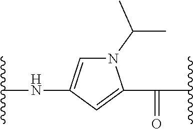 [SMILES: *Nc1cc(C(*)=O)n(C(C)C)c1] | — | — | + | + |
| (CTh) | 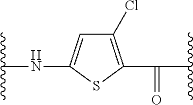 [SMILES: *Nc1cc(Cl)c(C(*)=O)s1] | + | + | — | — |
| (Dab) | 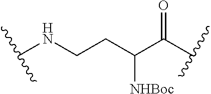 [SMILES: *NCCC(NC(=O)OC(C)(C)C)C(*)=O] | — | — | + | + |
| (gAH) | 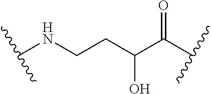 [SMILES: *NCCC(O)C(*)=O] | — | — | + | + |

| | | |
|---|---|---|
| (HpBi) | 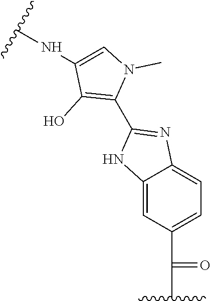 [SMILES: *Nc1cn(C)c(-c2nc3ccc(C(*)=O)cc3[nH]2)c1O] | WW* (bind to two nucleotides with same selectivity as Hp-Py) |
| (PhBi) | 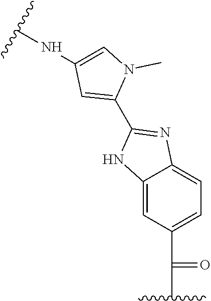 [SMILES: *Nc1cc(-c2nc3ccc(C(*)=O)cc3[nH]2)n(C)c1] | WW* (bind to two nucleotides with same selectivity as Py-Py) |
| (ImBi) | 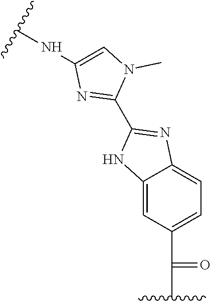 [SMILES: *Nc1cn(C)c(-c2nc3ccc(C(*)=O)cc3[nH]2)n1] | GW* (bind to two nucleotides with same selectivity as Im-Py) |

*The subunit HpBi, ImBi, and PyBi function as a conjugate of two monomer subunits and bind to two nucleotides. The binding property of HpBi, ImBi, and PyBi corresponds to Hp-Py, Im-Py, and Py-Py respectively.

TABLE 1B
Representative base pairing for hairpin polyamide.

| Nucleotide pair | G•C | C•G | T•A | A•T |
|---|---|---|---|---|
| Im/β | + | − | − | − |
| β/Im | − | + | − | − |
| Py/ β | − | − | + | + |
| β/Py | − | − | + | + |
| β/ β | − | − | + | + |
| Py/Py | − | − | + | + |
| Im/Im | − | − | − | − |
| Im/Py | + | − | − | − |
| Py/Im | − | + | − | − |
| Th/Py | − | − | + | − |
| Py/Th | − | − | − | + |
| Th/Im | + | − | − | − |
| Im/Th | − | + | − | − |
| β/Th | − | − | + | − |
| Th/β | − | − | − | + |
| Hp/Py, | − | − | + | − |
| Py/Hp, | − | − | − | + |
| Hp/Im | + | − | − | − |
| Im/Hp | − | + | − | − |
| Tn/Py | − | − | + | + |
| Py/Tn, | − | − | + | + |
| Ht/Py, | − | − | + | + |
| Py/Ht, | − | − | + | + |
| Bi/Py, | − | − | + | + |
| Py/Bi, | − | − | + | + |
| β/Bi | − | − | + | + |
| Bi/β | − | − | + | + |
| Bi/Im, | − | + | − | − |
| Im/Bi, | + | − | − | − |
| Tp/Py, | − | − | + | + |
| Py/Tp, | − | − | + | + |
| β/Tp | − | − | + | + |
| Tp/β | − | − | + | + |
| Tp/Im, | − | + | − | − |
| Im/Tp | + | − | − | − |
| Tp/Tp | − | − | + | + |
| Tp/Tn | − | − | + | + |
| Tn/Tp | − | − | + | + |
| Hz/Py, | − | − | + | − |
| Py/Hz, | − | − | − | + |
| Ip/Py | + | − | − | − |
| Py/Ip, | − | + | − | − |
| Bi/Hz, | − | − | + | + |
| Hz/Bi, | − | − | + | + |
| Bi/Bi | − | + | + | + |
| Th/Py, | − | − | + | + |
| Py/Th | − | − | + | + |
| Im/gAB | + | − | − | − |
| gAB/Im | − | + | − | − |
| Py/ gAB | + | − | − | − |
| gAB/Py | − | + | − | − |
| gAB/ β | − | − | + | + |
| β/gAB | − | − | + | + |
| Im/Dp | + | − | − | − |
| Dp/Im | − | + | − | − |
| Py/ Dp | − | − | + | + |
| Dp/Py | − | − | + | + |
| Dp/β | − | − | + | + |

Each of HpBi, ImBi, and PyBi can bind to two nucleotides and have binding properties corresponding to Hp-Py, Im-Py, and Py-Py respectively. HpBi, ImBi, and PyBi can be paired with two monomer subunits or with themselves in a hairpin structure to bind to two nucleotide pairs.

The monomer subunits of the polyamide compound can be strung together based on the pairing principles shown in Table 1A and Table 1B. The monomer subunits of the polyamide compound can be strung together based on the pairing principles shown in Table 1C.

Table 1C shows an example of the monomer subunits that can bind to the specific nucleotide. The polyamide can have several monomer subunits strung together, with a monomer subunit selected from each row. For example, the polyamide can include Py-Py-Im that binds to CAG, with Py is selected from the C column, Py is selected from the A column, and Im selected from the first G column. The polyamide can be any combinations of the subunits of CAGCAG, with a subunit selected from each column in Table 1C, wherein the subunits are strung together following the CAG binding order.

Tn addition, the polyamide portion of the compound can also include a partial or multiple sets of the five subunits, such as 1.5, 2, 2.5, 3, 3.5, or 4 sets of the three subunits. The polyamide portion of the compound can include 3, 4, 5, 6, 7, 8, 9, 10, 12, 14, and 16 monomer subunits.

The polyamide portion of the compound can include monomer subunits that bind to 2, 3, 4, or 5 nucleotides of CAG. For example, the polyamide can bind to CA, CAG, AGC, CAGC, CAGCA, or CAGCAG. The polyamide portion of the compound can include monomer subunits that bind to 3, 4, 5, 6, 7, 8, 9, or 10 nucleotides of CAG repeat..

The monomer subunit, when positioned as a terminal unit, does not have an amine, carbonyl, or a carboxylic acid group at the terminal. The amine or carboxylic acid group in the terminal is replaced by a hydrogen. For example, Py, when used as a terminal unit, is understood to have the structure of

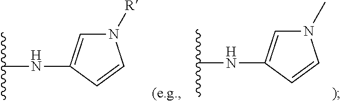

and Im, when positioned as a terminal unit, is understood to have the structure of

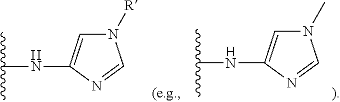

TABLE 1C
Examples of monomer subunits in a
linear polyamide that binds to CAG.

| Nucleotide | C | A | G |
|---|---|---|---|
| Subunits that | Py or PyT | Py | Im or ImT |
| bind to a | iIm or iImT | Th | iIm or iImT |
| nucleotide | PEG | Pz | PEG |
| | CTh | Tp | CTh |
| | Alx | PEG | Nt |
| | | β | iPTA |
| | | iPP | Ip |
| | | Da | CTh |
| | | Dp | |
| | | Dab | |
| | | gAH | |

The polyamide compound can also include a hairpin polyamide having subunits that are strung together based on the pairing principle shown in Table 1B. Table ID shows some examples of the monomer subunit pairs that selectively bind to the nucleotide pair.

Because the target gene can include multiple nucleotide repeats comprising CAG, the subunits can be strung together to bind at least two, three, four, five, six, seven, eight, nine, or ten nucleotides in one or more CAG repeat (e.g., CAGCAG). For example, the polyamide compound can bind to the CAG repeat by binding to a partial copy, a full copy, or multiple repeats comprising CAG such as CA, CAG, AGC, CAGC, CAGCA, or CAGCAG. For example, the polyamide compound can include Im-Im-Im-Im-β-β-W-Im-Im-β-Py-β-Py that binds to GGGGCC and its complementary nucleotides on a double strand DNA, in which the Im/Py pair binds to the G·C, the Im/s pair binds to G·C, the Im/Py pair binds to G·C, the Im/β binds to G·C, and β/Im binds to C·G; and β/Im binds to C·G. In one example Py-β-Im-β-W-Im-Py-Py-Im that binds to CTGC and its complementary nucleotides on a double strand DNA, in which Py/Im pair binds to C·G, p/Py pair binds to T·A, Im/Im pair binds to C·G, and p/Py pair binds to C·G. W can be an aliphatic amino acid residue such as gAB or other appropriate spacers as shown in Table 4. In another example, the polyamide compound can include Im-Py-Py-Im-Py-gAB-Im-Py-Py-Im-0 that binds to GCTGC and its complementary nucleotides on a double strand DNA, in which the Im/β pair binds to G·C, the Py/Im pair binds to C·G, the Py/Py binds to T·A, Im/Py pair binds to the GC, and Py/Im binds to C·G. In another example, Im-Py-Py-Im-Py-gAB-Im-Py-Py binds to GCTGC with a part of the complementary nucleotides (ACG) on the double strand DNA, in which Im binds to G, Py binds to C, Py/Py binds to T·A, Im/Py binds to the C—C, and Py/Im binds to the C·G.

TABLE 1D
Examples of monomer pairs in a hairpin
polyamide that binds to CAG.

| Nucleotide | C•G | A•T | G•C |
|---|---|---|---|
| Subunit | β/Im | Py/ β | Im/β |
| pairs that | Py/Im | β/Py | Im/Py |
| selectively | Im/Th | β/ β | Th/Im |
| bind to a | Im/Hp | Py/Py | Hp/Im |
| nucleotide | Bi/Im | Py/Th | Im/Bi |
| | Tp/Im | Th/β | Im/Tp |
| | Py/Ip | Py/Hp, | Ip/Py |
| | Bi/Bi | Tn/Py | Im/gAB |
| | gAB/Im | Py/Tn, | Py/gAB |
| | gAB/Py | Ht/Py, | Im/Dp |
| | Dp/Im | Py/Ht, | |
| | | Bi/Py, | |
| | | Py/Bi, | |
| | | β/Bi | |
| | | Bi/β | |
| | | Tp/Py, | |
| | | Py/Tp, | |
| | | β/Tp | |
| | | Tp/β | |
| | | Tp/Tp | |
| | | Tp/Tn | |
| | | Tn/Tp | |
| | | Py/Hz, | |
| | | Bi/Hz, | |
| | | Hz/Bi, | |
| | | Bi/Bi | |
| | | Th/Py, | |
| | | Py/Th | |
| | | gAB/ β | |
| | | β/gAB | |
| | | Py/ Dp | |
| | | Dp/Py | |
| | | Dp/β | |

Recognition of a nucleotide repeat or DNA sequence by two antiparallel polyamide strands depends on a code of side-by-side aromatic amino acid pairs in the minor groove, usually oriented N to C with respect to the 5′ to 3′ direction of the DNA helix. Enhanced affinity and specificity of polyamide nucleotide binding is accomplished by covalently linking the antiparallel strands. The “hairpin motif” connects the N and C termini of the two strands with a W (e.g., gamma-aminobutyric acid unit (gamma-turn)) to form a folded linear chain.

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed

In some embodiments, non-limiting examples of the transcription modulator compounds described herein are presented in Table 2.

TABLE 2

| | |
|---|---|
| 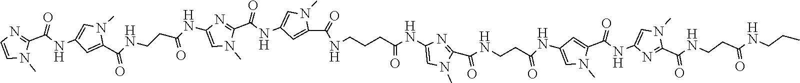 [SMILES: CCCNC(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C] | B-1 |
| 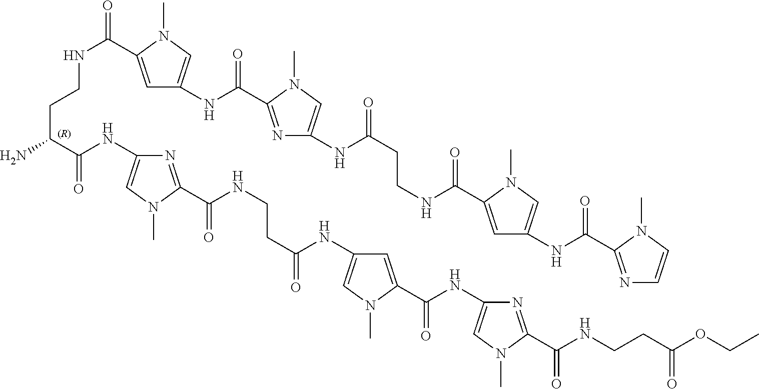 | B-2 |
| 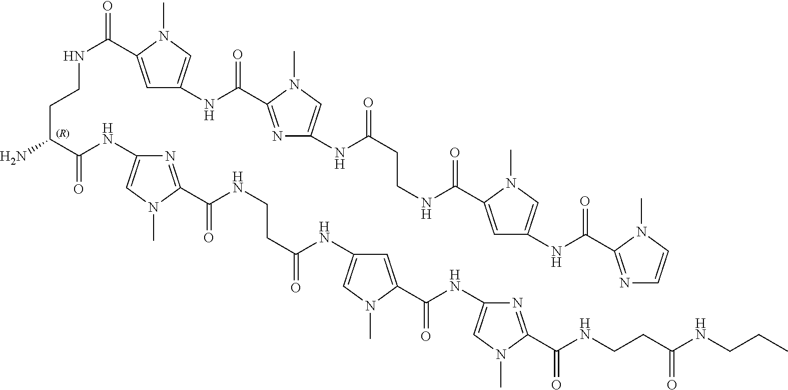 | B-3 |
| 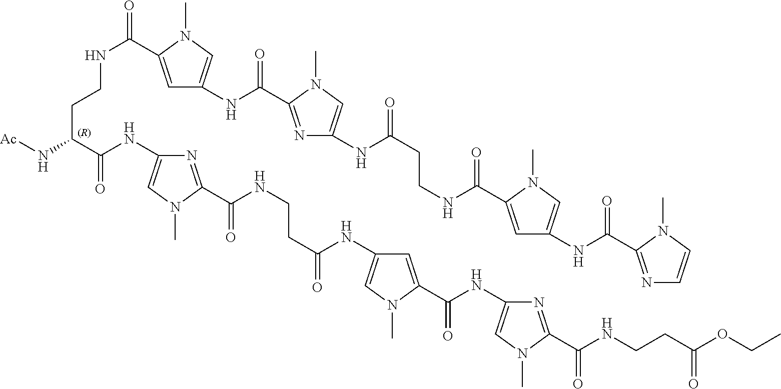 | B-4 |
| 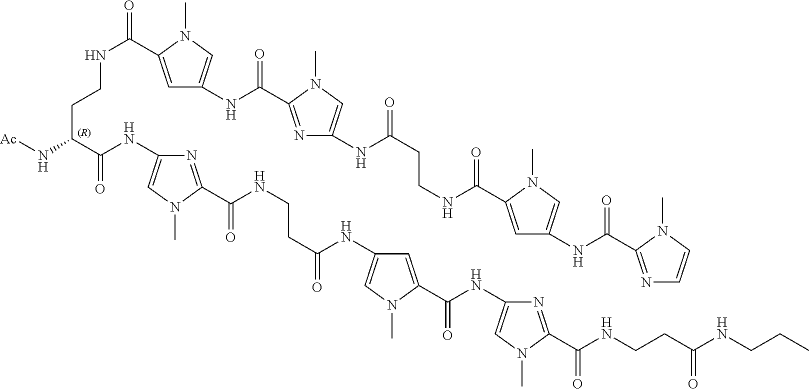 | B-5 |
| 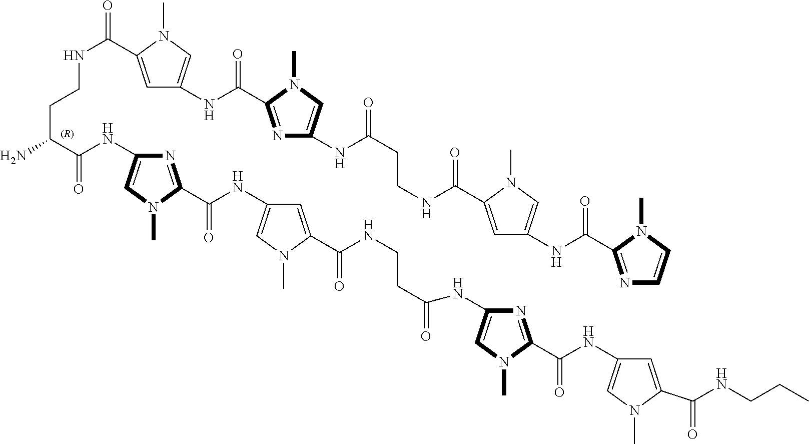 | B-6 |
| 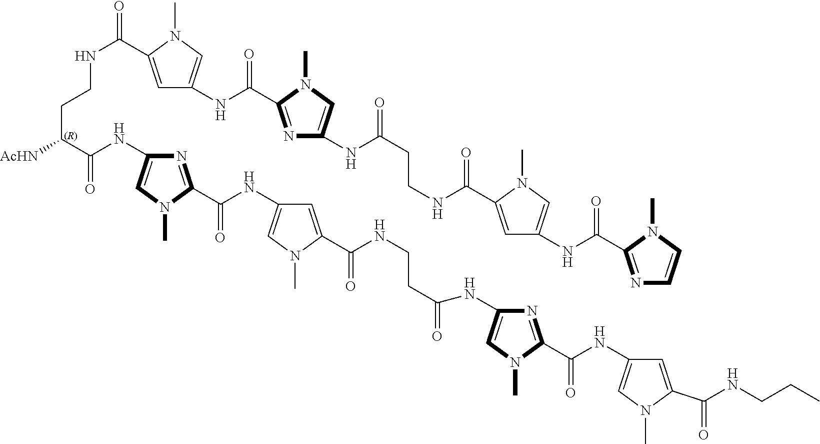 | B-7 |
| 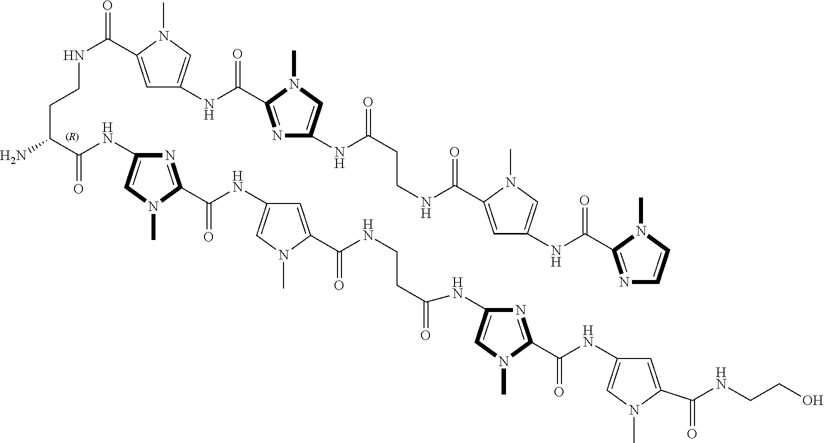 | B-8 |
| 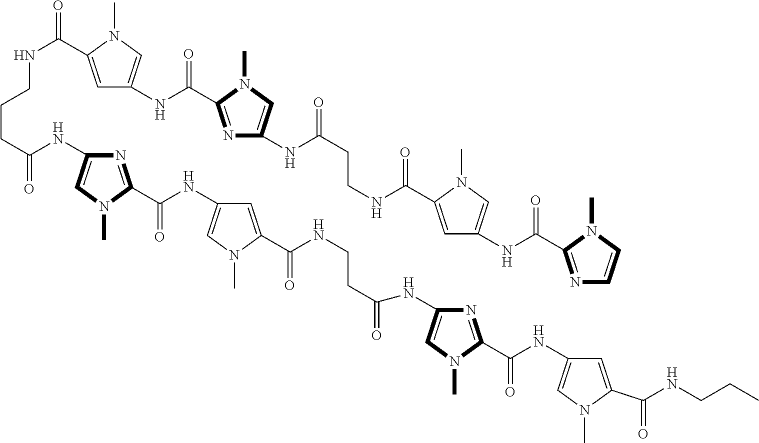 | B-9 |
| 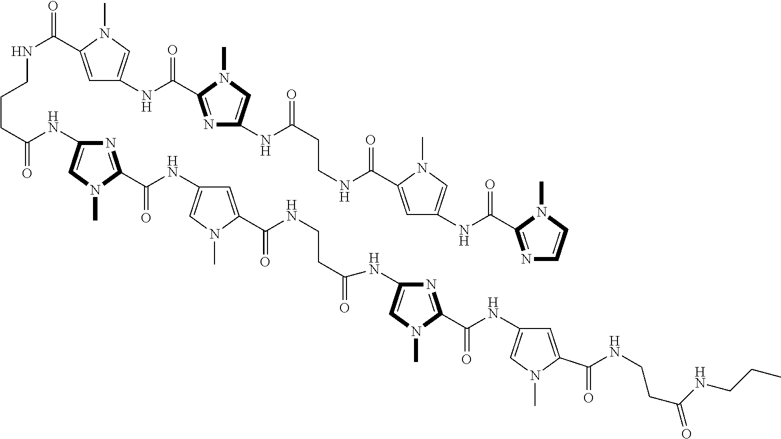 | B-10 |
| 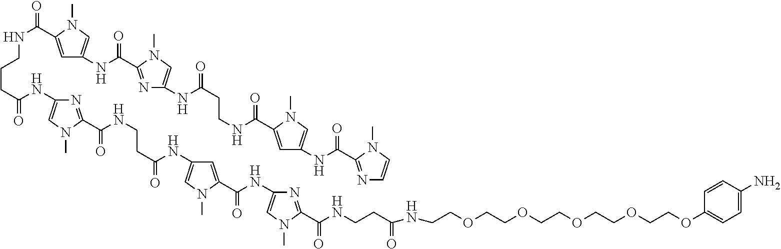 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)NCCOCCOCCOCCOCCOc4ccc(N)cc4)n3)n(C)c2)n1] | B-11 |
| 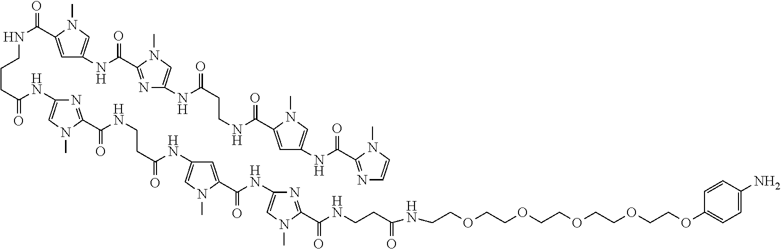 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)NCCOCCOCCOCCOCCOc4ccc(N)cc4)n3)n(C)c2)n1] | B-12 |
| 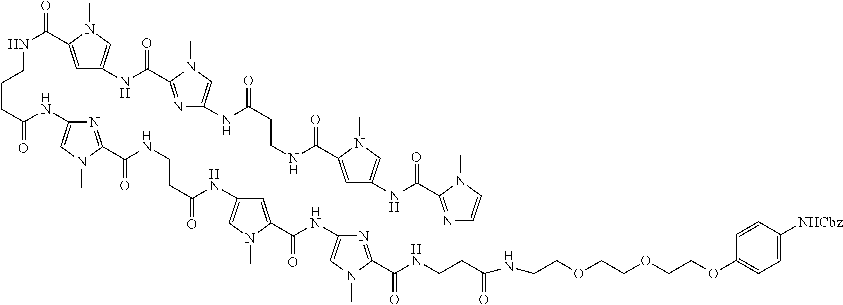 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)NCCOCCOCCOc4ccc(NC(=O)OCc5ccccc5)cc4)n3)n(C)c2)n1] | B-13 |
| 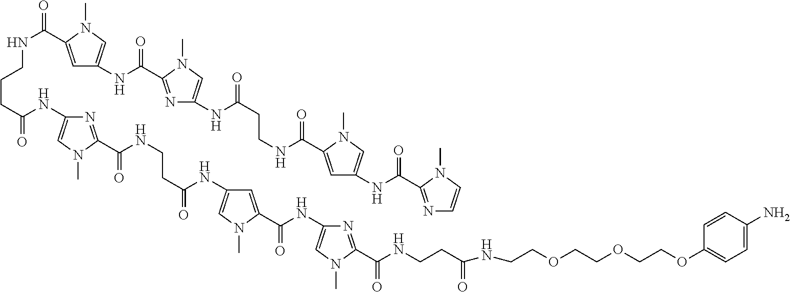 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)NCCOCCOCCOc4ccc(N)cc4)n3)n(C)c2)n1] | B-14 |
| 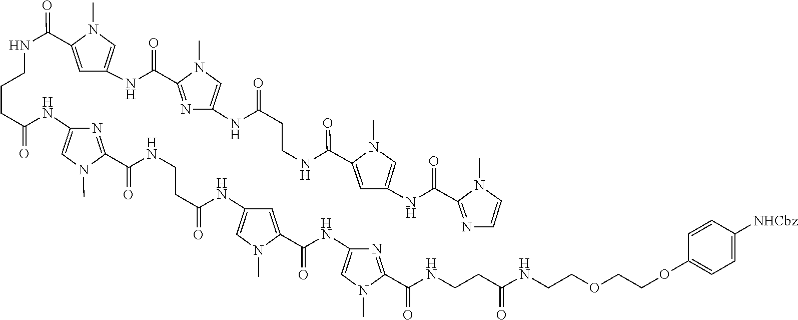 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)NCCOCCOc4ccc(NC(=O)OCc5ccccc5)cc4)n3)n(C)c2)n1] | B-15 |
| 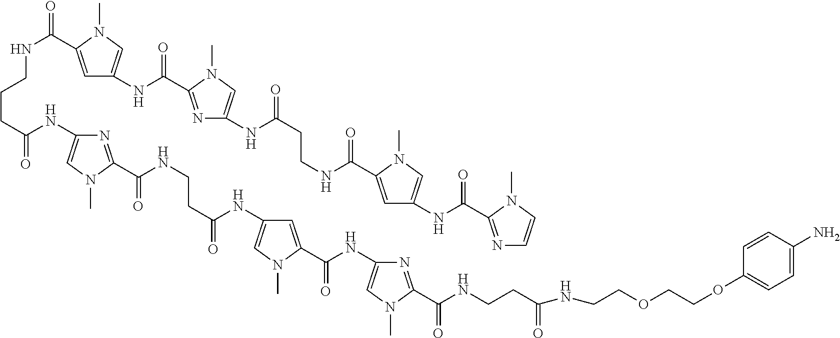 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)NCCOCCOc4ccc(N)cc4)n3)n(C)c2)n1] | B-16 |
| 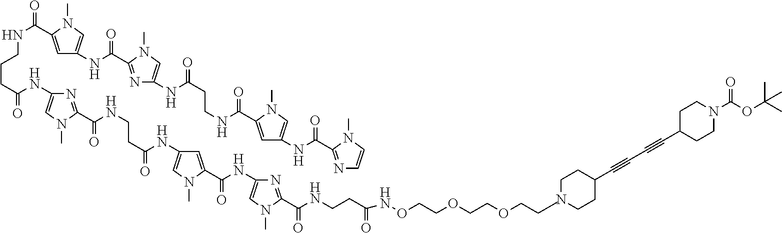 | B-17 |
| 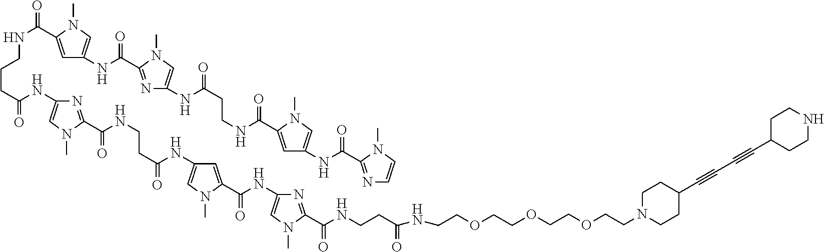 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)NCCOCCOCCOCCN4CCC(C#CC#CC5CCNCC5)CC4)n3)n(C)c2)n1] | B-18 |
| 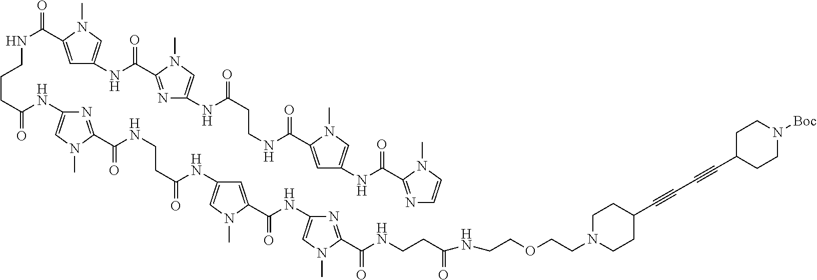 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)NCCOCCN4CCC(C#CC#CC5CCN(C(=O)OC(C)(C)C)CC5)CC4)n3)n(C)c2)n1] | B-19 |
| 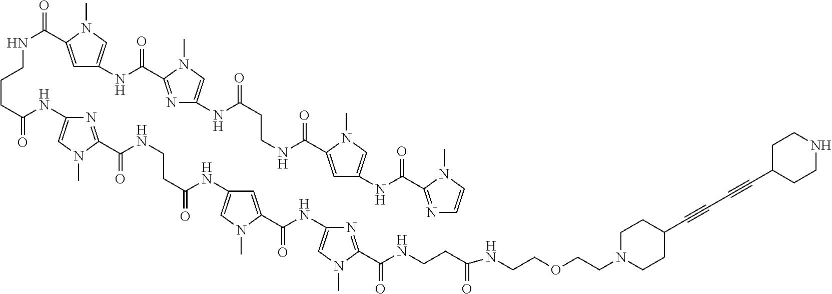 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)NCCOCCN4CCC(C#CC#CC5CCNCC5)CC4)n3)n(C)c2)n1] | B-20 |
| 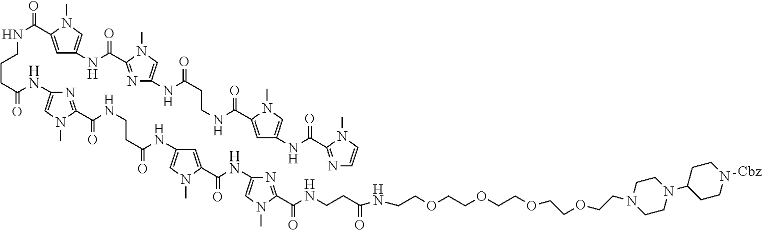 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)NCCOCCOCCOCCOCCN4CCN(C5CCN(C(=O)OCc6ccccc6)CC5)CC4)n3)n(C)c2)n1] | B-21 |
| 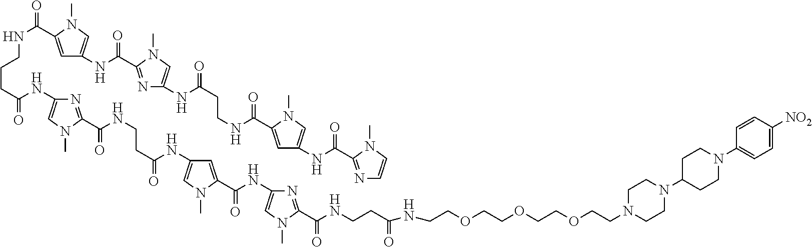 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)NCCOCCOCCOCCN4CCN(C5CCN(c6ccc([N+](=O)[O-])cc6)CC5)CC4)n3)n(C)c2)n1] | B-22 |
| 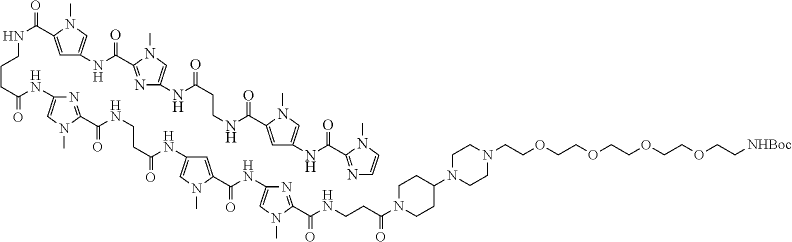 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)N4CCC(N5CCN(CCOCCOCCOCCOCCNC(=O)OC(C)(C)C)CC5)CC4)n3)n(C)c2)n1] | B-23 |
| 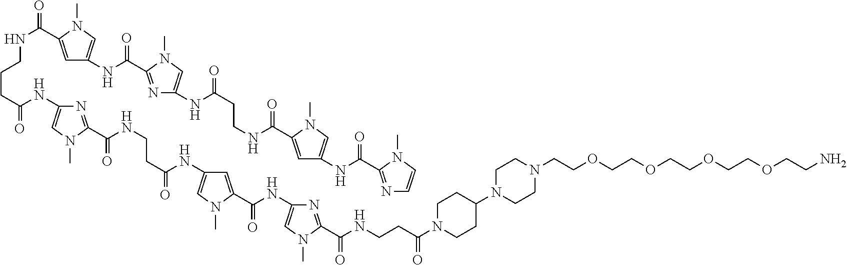 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)N4CCC(N5CCN(CCOCCOCCOCCOCCN)CC5)CC4)n3)n(C)c2)n1] | B-24 |
| 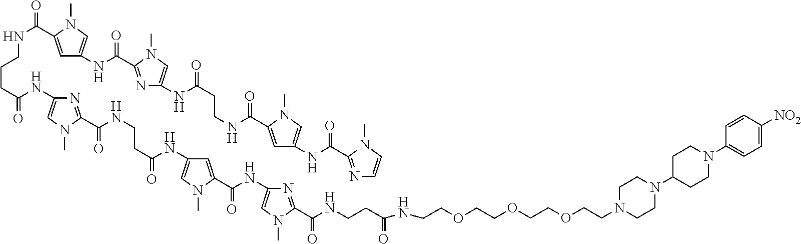 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)NCCOCCOCCOCCN4CCN(C5CCN(c6ccc([N+](=O)[O-])cc6)CC5)CC4)n3)n(C)c2)n1] | B-25 |
| 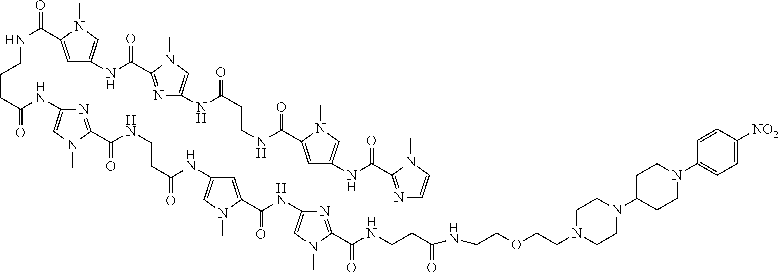 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)NCCOCCN4CCN(C5CCN(c6ccc([N+](=O)[O-])cc6)CC5)CC4)n3)n(C)c2)n1] | B-26 |
| 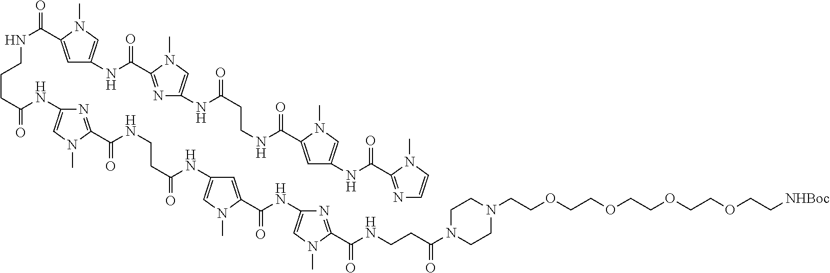 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)N4CCN(CCOCCOCCOCCOCCNC(=O)OC(C)(C)C)CC4)n3)n(C)c2)n1] | B-27 |
| 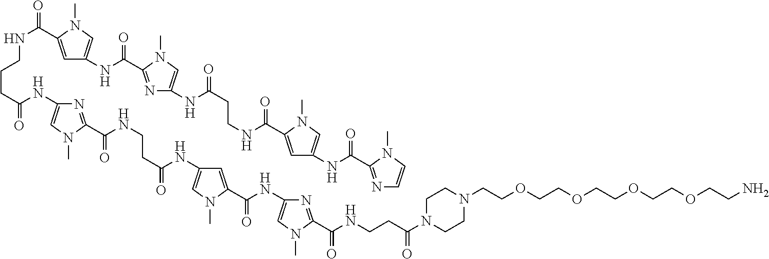 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)N4CCN(CCOCCOCCOCCOCCN)CC4)n3)n(C)c2)n1] | B-28 |
| 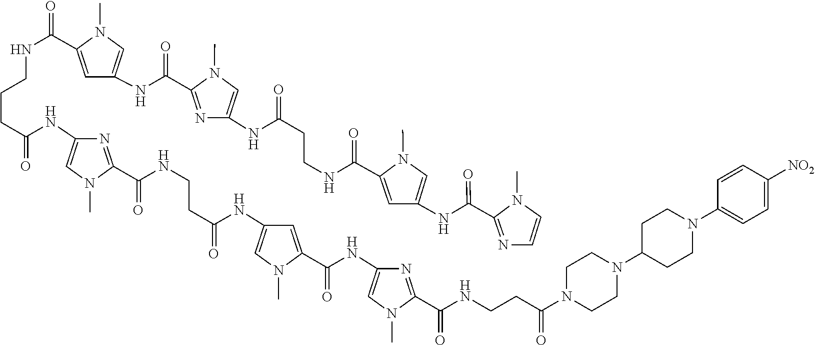 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)N4CCN(C5CCN(c6ccc([N+](=O)[O-])cc6)CC5)CC4)n3)n(C)c2)n1] | B-29 |
| 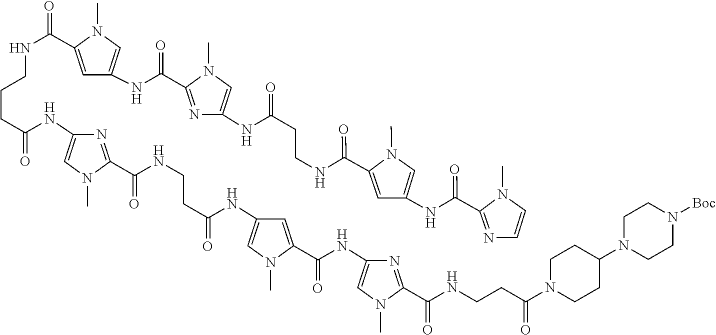 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)N4CCC(N5CCN(C(=O)OC(C)(C)C)CC5)CC4)n3)n(C)c2)n1] | B-30 |
| 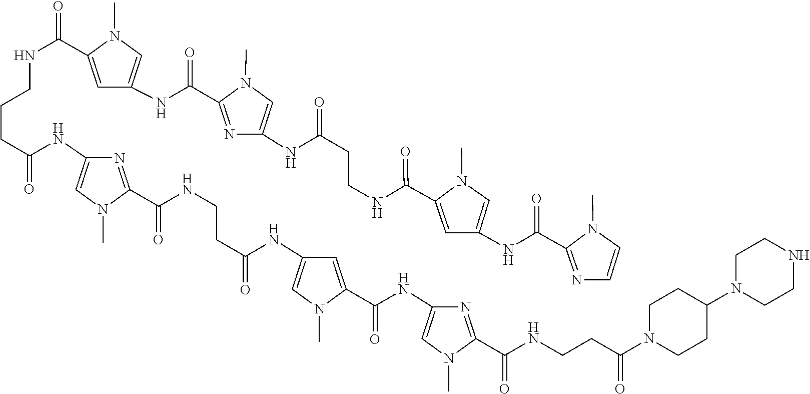 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)N4CCC(N5CCNCC5)CC4)n3)n(C)c2)n1] | B-31 |
| 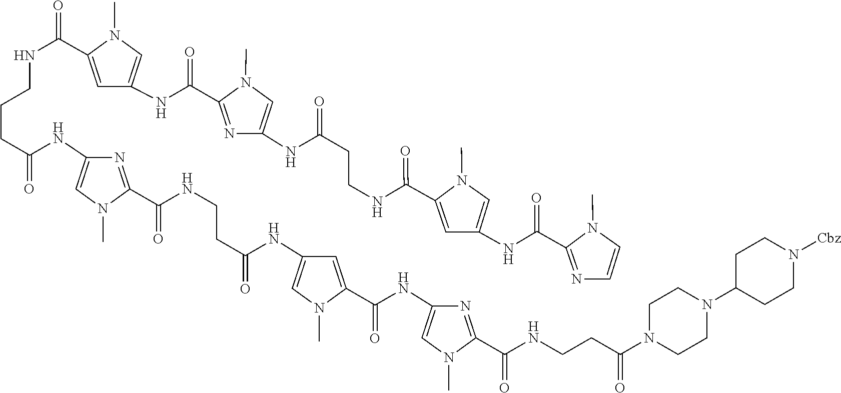 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)N4CCN(C5CCN(C(=O)OCc6ccccc6)CC5)CC4)n3)n(C)c2)n1] | B-32 |
| 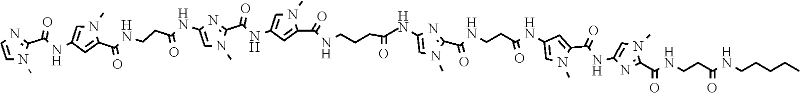 | B-33 |
| 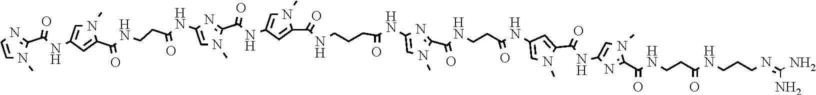 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)NCCCN=C(N)N)n3)n(C)c2)n1] | B-34 |
| 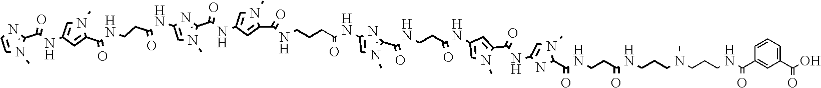 [SMILES: CN(CCCNC(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C)CCCNC(=O)c1cccc(C(=O)O)c1] | B-35 |
| 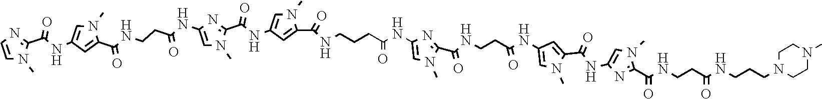 [SMILES: CN1CCN(CCCNC(=O)CCNC(=O)c2nc(NC(=O)c3cc(NC(=O)CCNC(=O)c4nc(NC(=O)CCCNC(=O)c5cc(NC(=O)c6nc(NC(=O)CCNC(=O)c7cc(NC(=O)c8nccn8C)cn7C)cn6C)cn5C)cn4C)cn3C)cn2C)CC1] | B-36 |
| 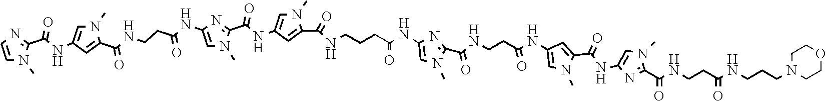 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)NCCCN4CCOCC4)n3)n(C)c2)n1] | B-37 |
| 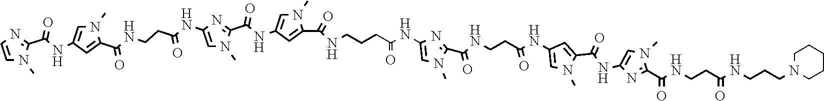 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)NCCCN4CCCCC4)n3)n(C)c2)n1] | B-38 |
| 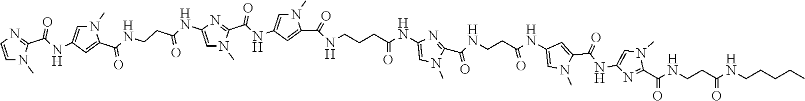 [SMILES: CCCCCNC(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C] | B-39 |
| 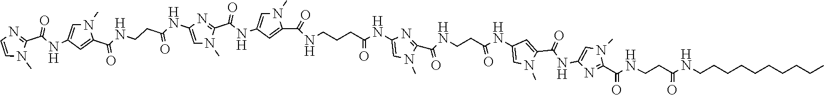 [SMILES: CCCCCCCCCCNC(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C] | B-40 |
| 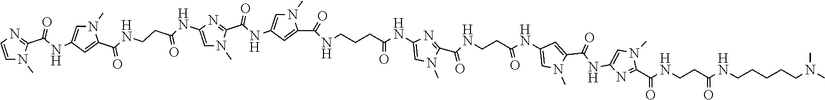 [SMILES: CN(C)CCCCCNC(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C] | B-41 |
| 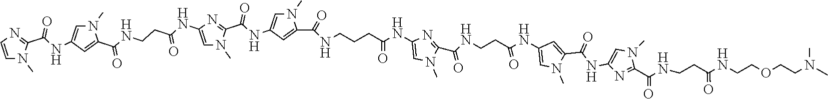 [SMILES: CN(C)CCOCCNC(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C] | B-42 |
| 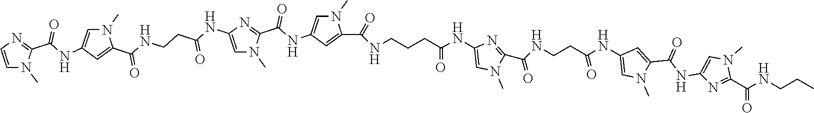 [SMILES: CCCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C] | B-43 |
| 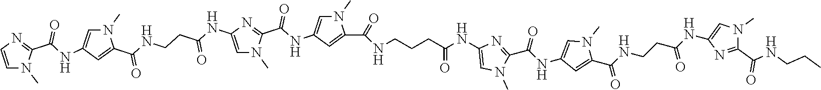 [SMILES: CCCNC(=O)c1nc(NC(=O)CCNC(=O)c2cc(NC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C] | B-44 |
| 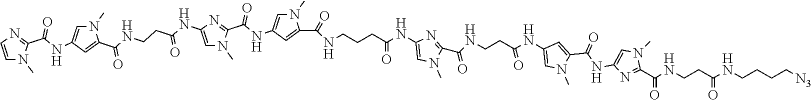 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)NCCCCN=[N+]=[N-])n3)n(C)c2)n1] | B-45 |
| 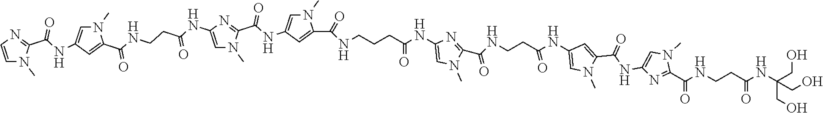 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)NC(CO)(CO)CO)n3)n(C)c2)n1] | B-46 |
| 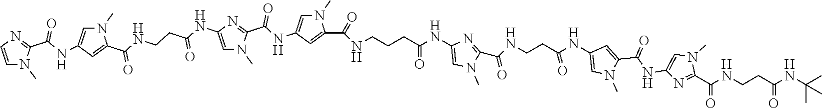 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)NC(C)(C)C)n3)n(C)c2)n1] | B-47 |
| 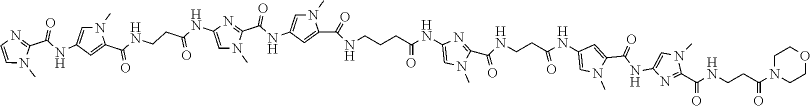 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)N4CCOCC4)n3)n(C)c2)n1] | B-48 |
| 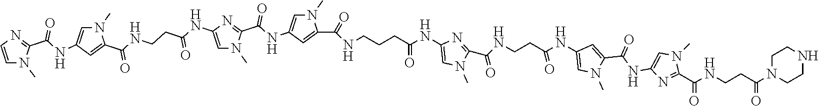 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)N4CCNCC4)n3)n(C)c2)n1] | B-49 |
| 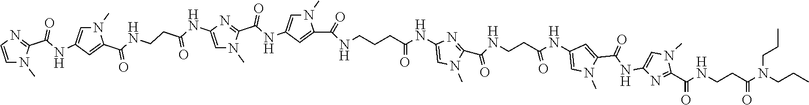 [SMILES: CCCN(CCC)C(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C] | B-50 |
| 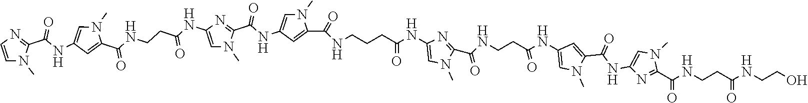 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)NCCO)n3)n(C)c2)n1] | B-51 |
| 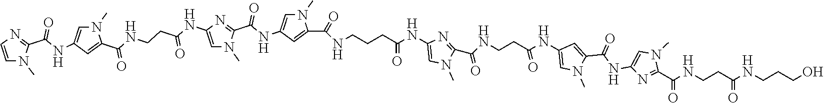 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)NCCCO)n3)n(C)c2)n1] | B-52 |
| 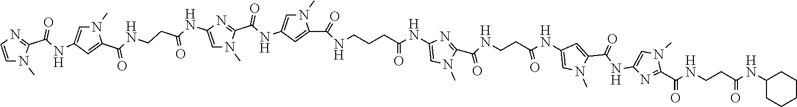 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)NC4CCCCC4)n3)n(C)c2)n1] | B-53 |
| 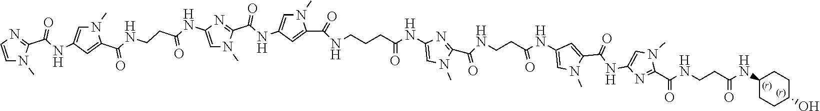 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)N[C@H]4CC[C@H](O)CC4)n3)n(C)c2)n1] | B-54 |
| 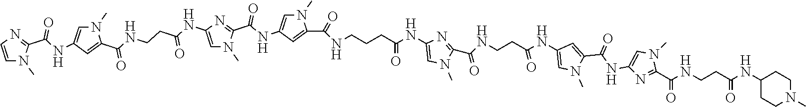 [SMILES: CN1CCC(NC(=O)CCNC(=O)c2nc(NC(=O)c3cc(NC(=O)CCNC(=O)c4nc(NC(=O)CCCNC(=O)c5cc(NC(=O)c6nc(NC(=O)CCNC(=O)c7cc(NC(=O)c8nccn8C)cn7C)cn6C)cn5C)cn4C)cn3C)cn2C)CC1] | B-55 |
| 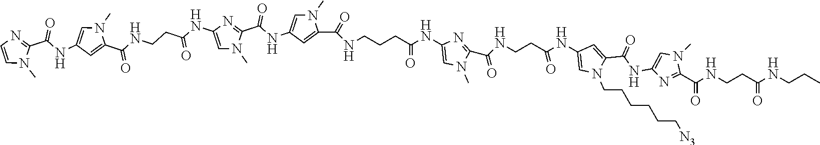 [SMILES: CCCNC(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2CCCCCCN=[N+]=[N-])cn1C] | B-56 |
| 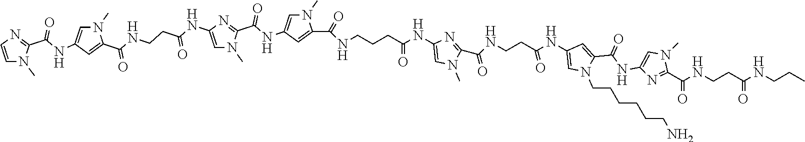 [SMILES: CCCNC(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2CCCCCCN)cn1C] | B-57 |
| 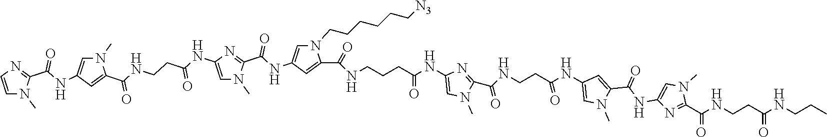 [SMILES: CCCNC(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4CCCCCCN=[N+]=[N-])cn3C)cn2C)cn1C] | B-58 |
| 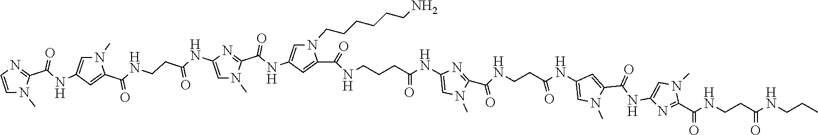 [SMILES: CCCNC(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4CCCCCCN)cn3C)cn2C)cn1C] | B-59 |
| 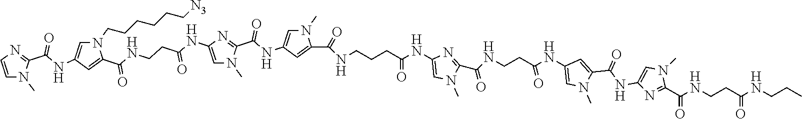 [SMILES: CCCNC(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6CCCCCCN=[N+]=[N-])cn5C)cn4C)cn3C)cn2C)cn1C] | B-60 |
| 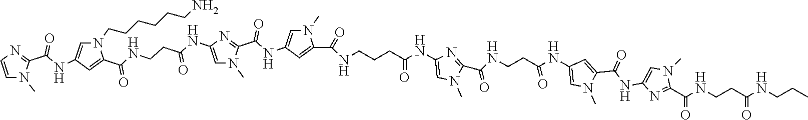 [SMILES: CCCNC(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6CCCCCCN)cn5C)cn4C)cn3C)cn2C)cn1C] | B-61 |
| 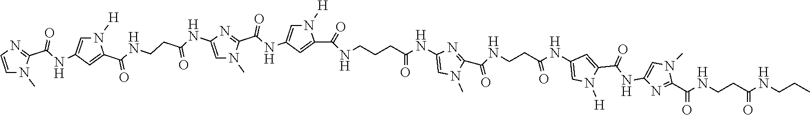 [SMILES: CCCNC(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)c[nH]6)cn5C)c[nH]4)cn3C)c[nH]2)cn1C] | B-62 |
| 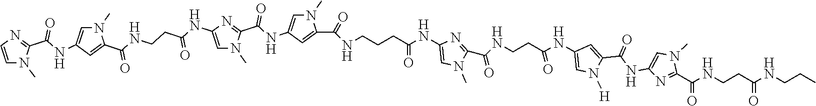 [SMILES: CCCNC(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)c[nH]2)cn1C] | B-63 |
| 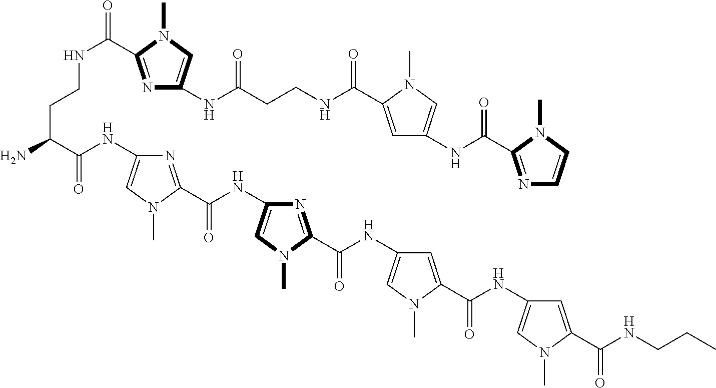 | B-64 |
| 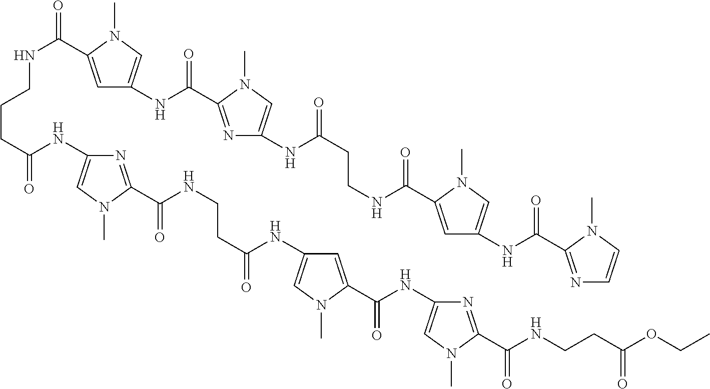 [SMILES: CCOC(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C] | B-65 |
| 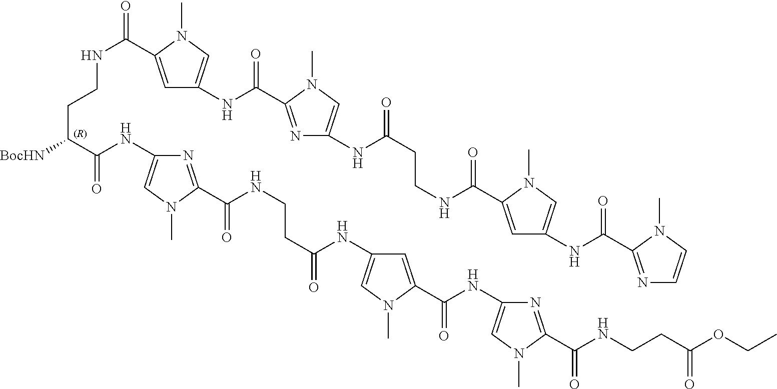 | B-66 |
| 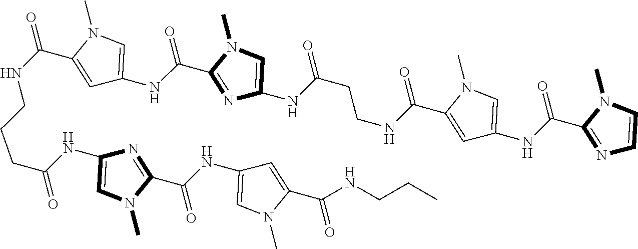 | B-67 |
| 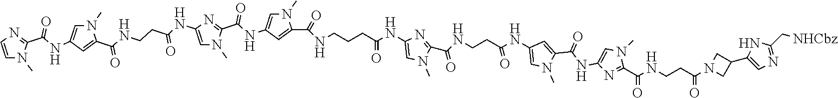 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)N4CC(c5cnc(CNC(=O)OCc6ccccc6)[nH]5)C4)n3)n(C)c2)n1] | B-68 |
| 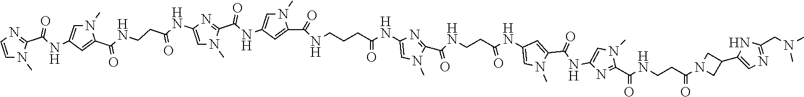 [SMILES: CN(C)Cc1ncc(C2CN(C(=O)CCNC(=O)c3nc(NC(=O)c4cc(NC(=O)CCNC(=O)c5nc(NC(=O)CCCNC(=O)c6cc(NC(=O)c7nc(NC(=O)CCNC(=O)c8cc(NC(=O)c9nccn9C)cn8C)cn7C)cn6C)cn5C)cn4C)cn3C)C2)[nH]1] | B-69 |
| 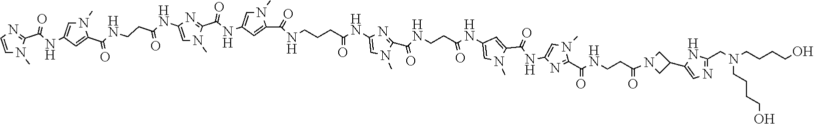 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)N4CC(c5cnc(CN(CCCCO)CCCCO)[nH]5)C4)n3)n(C)c2)n1] | B-70 |
| 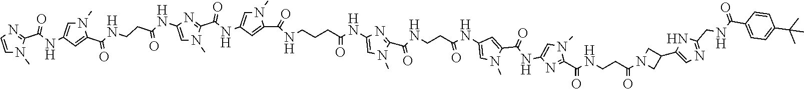 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)N4CC(c5cnc(CNC(=O)c6ccc(C(C)(C)C)cc6)[nH]5)C4)n3)n(C)c2)n1] | B-71 |
| 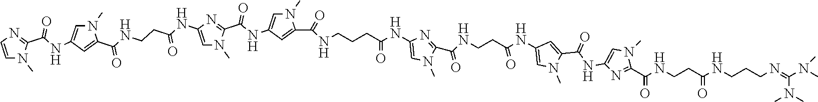 [SMILES: CN(C)C(=NCCCNC(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C)N(C)C] | B-72 |
| 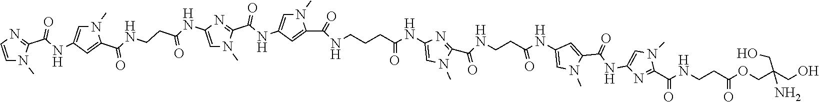 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)OCC(N)(CO)CO)n3)n(C)c2)n1] | B-73 |
| 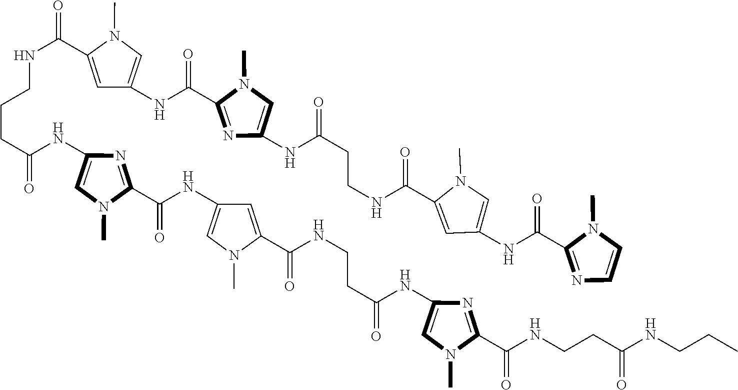 | B-74 |
| 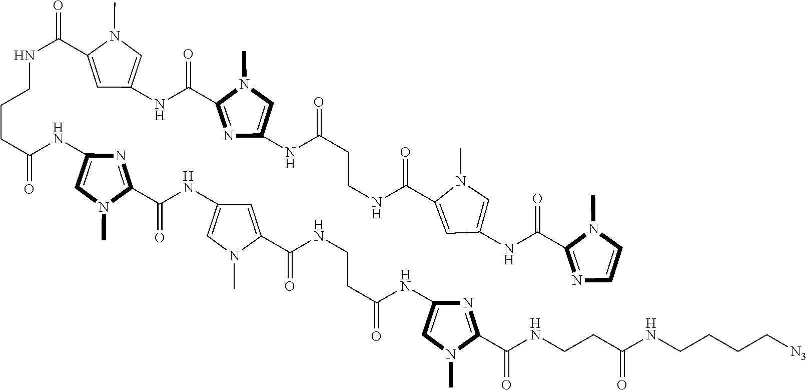 | B-75 |
| 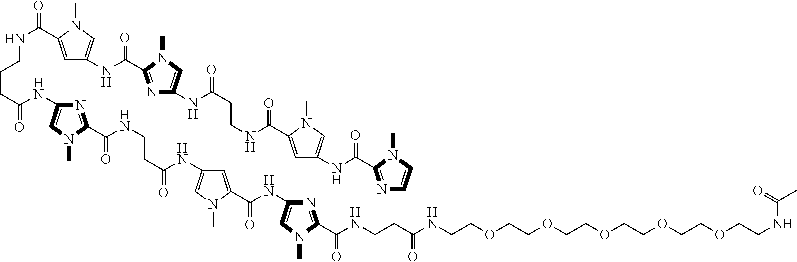 | B-76 |
| 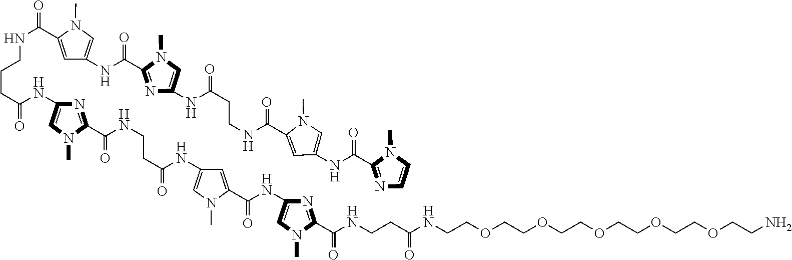 | B-77 |
| 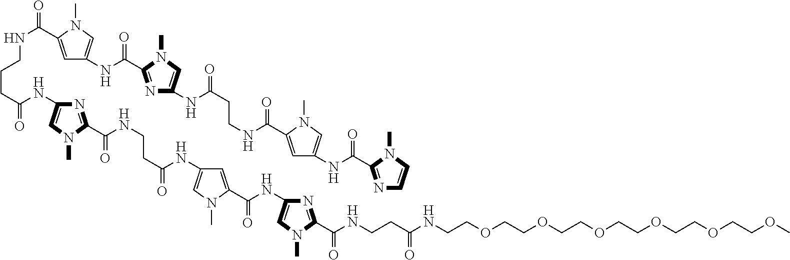 | B-78 |
| 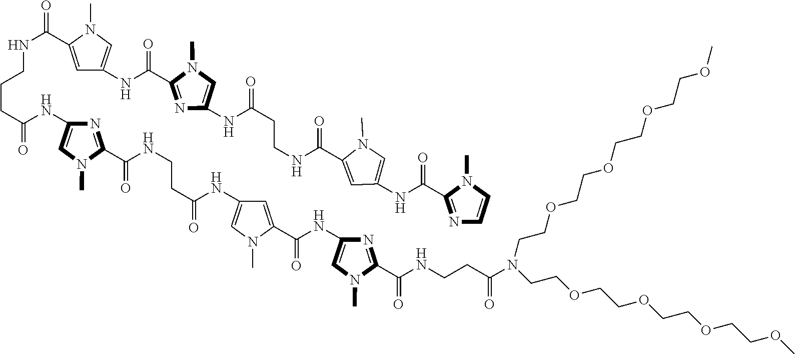 | B-79 |
| 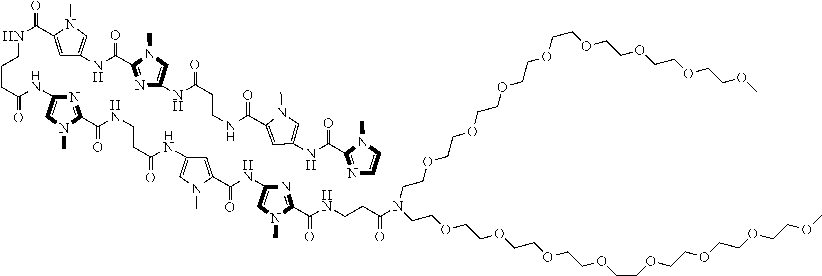 | B-80 |
| 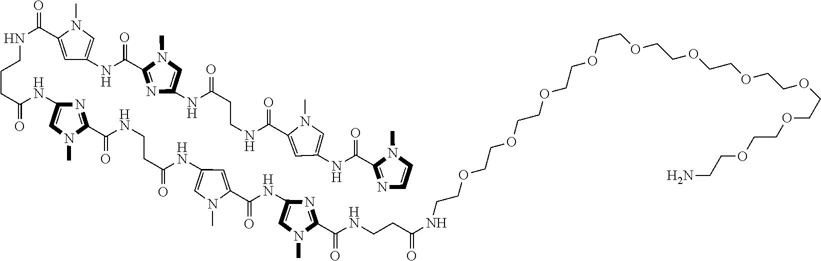 | B-81 |
| 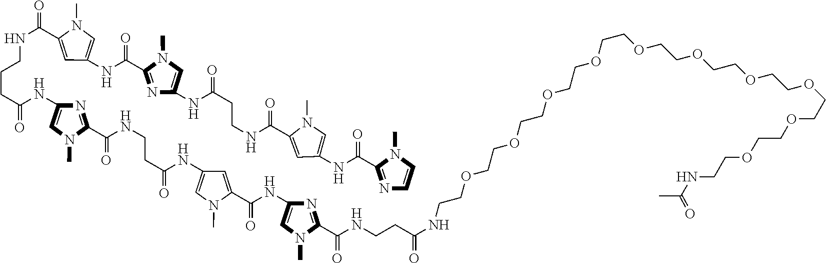 | B-82 |
| 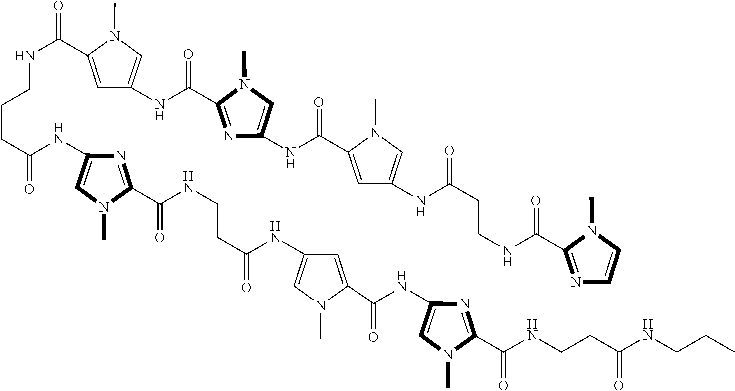 | B-83 |
| 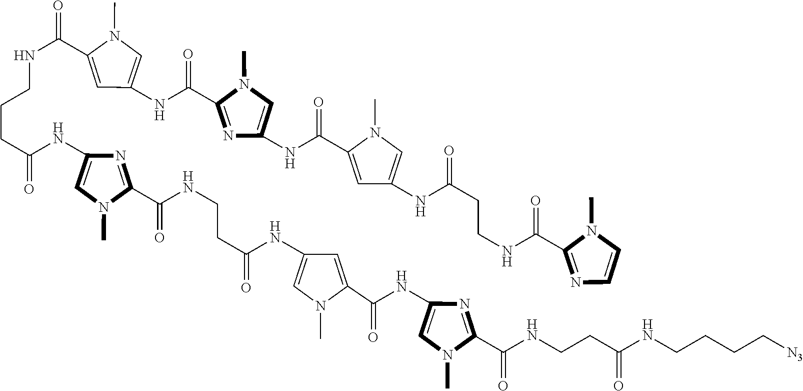 | B-84 |
| 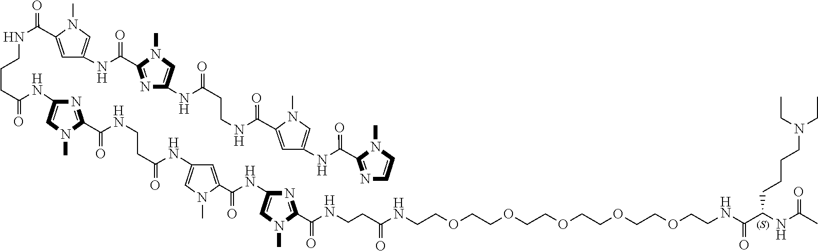 | B-85 |
| 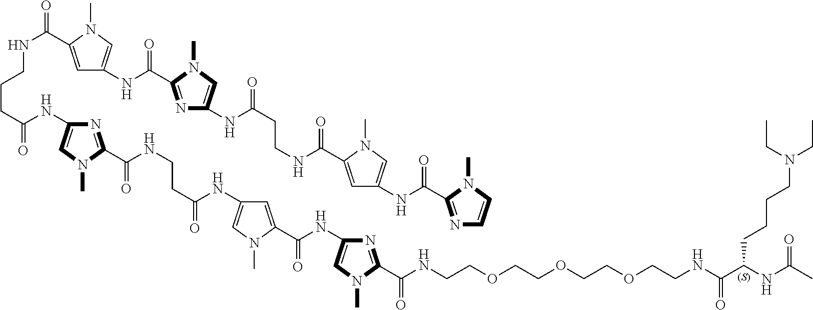 | B-86 |
| 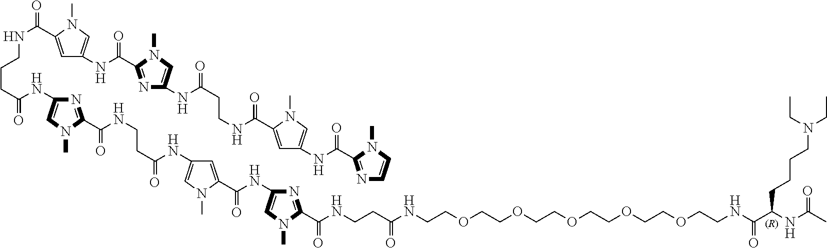 | B-87 |
| 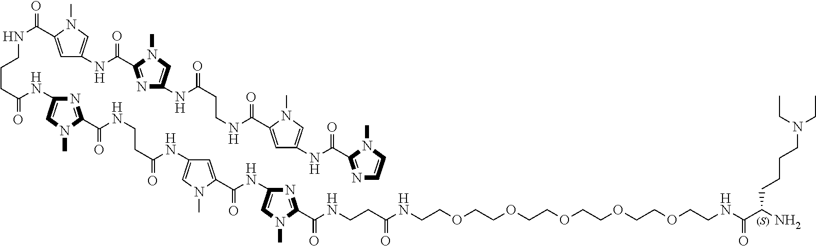 | B-88 |
| 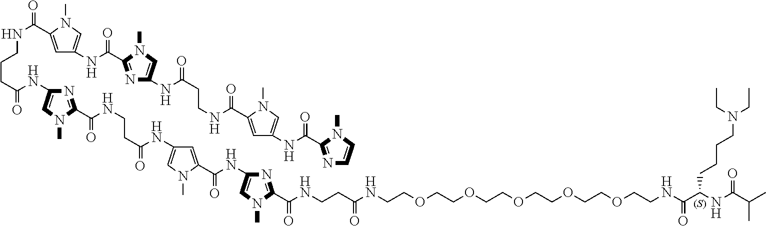 | B-89 |
| 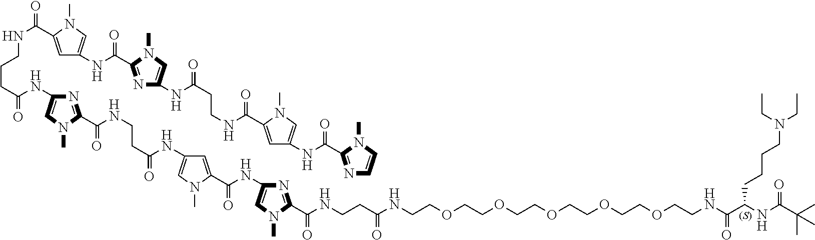 | B-90 |
| 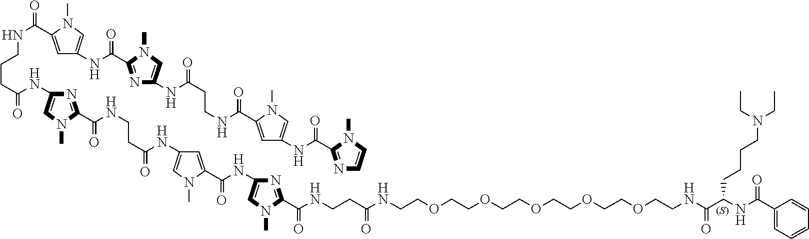 | B-91 |
| 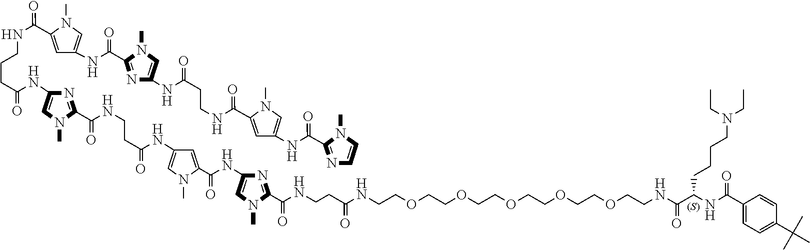 | B-92 |
| 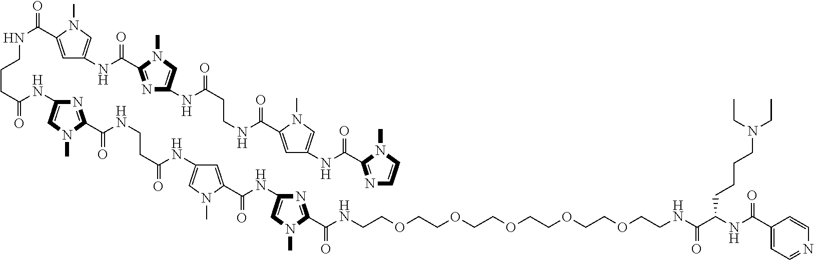 | B-93 |
| 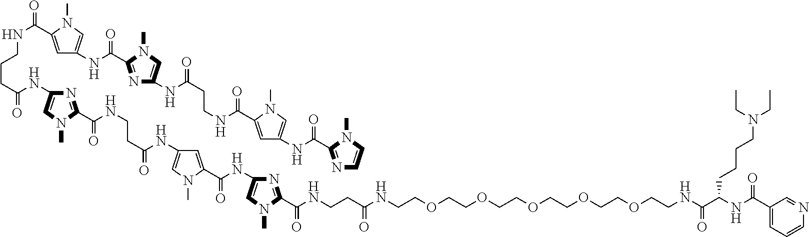 | B-94 |
| 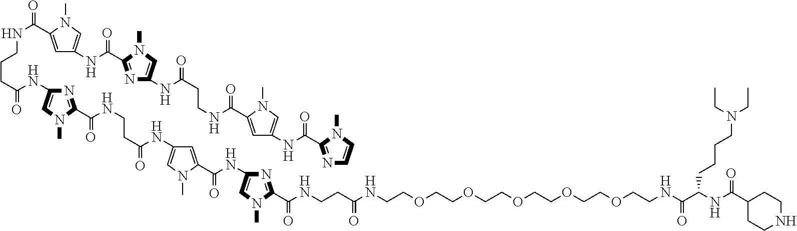 | B-95 |
| 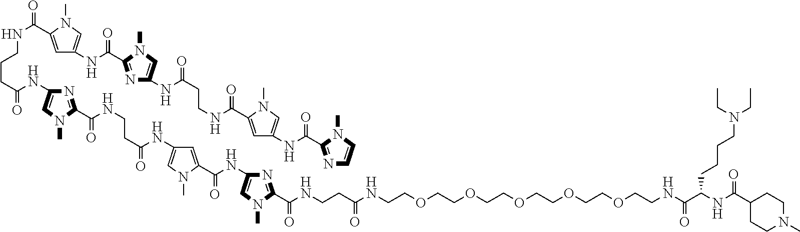 | B-96 |
| 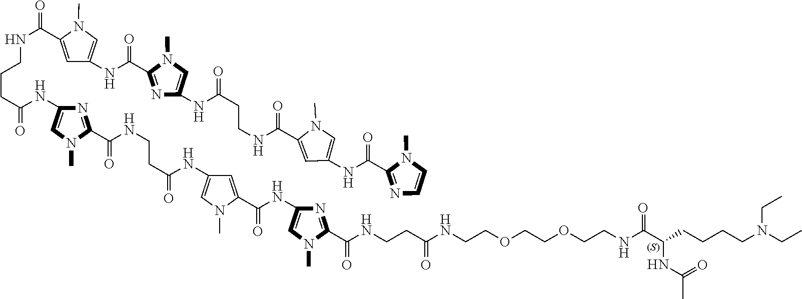 | B-97 |
| 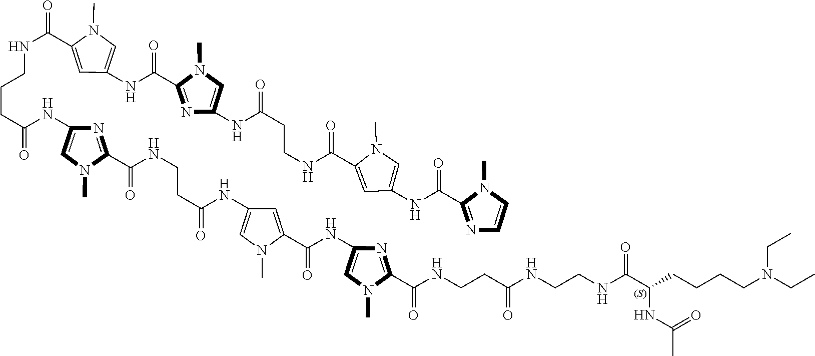 | B-98 |
| 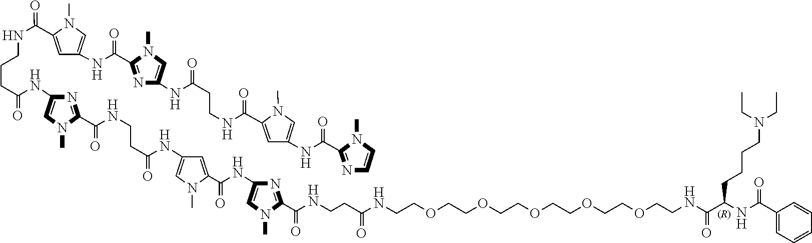 | B-99 |
| 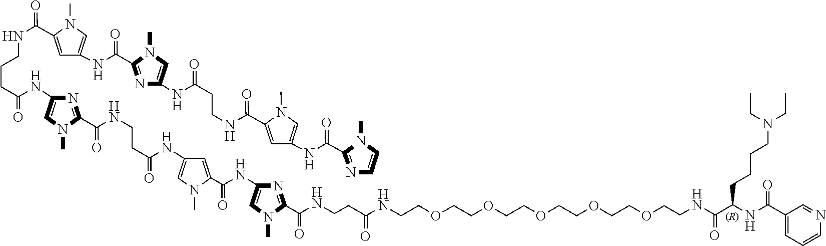 | B-100 |
| 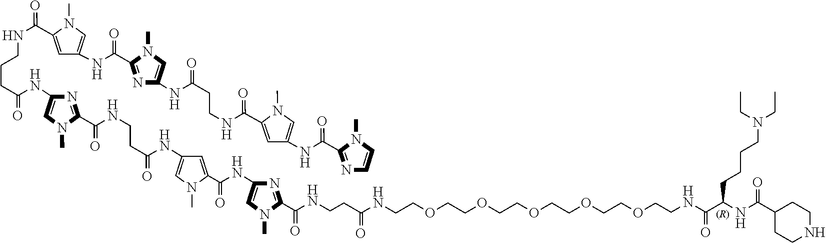 | B-101 |
| 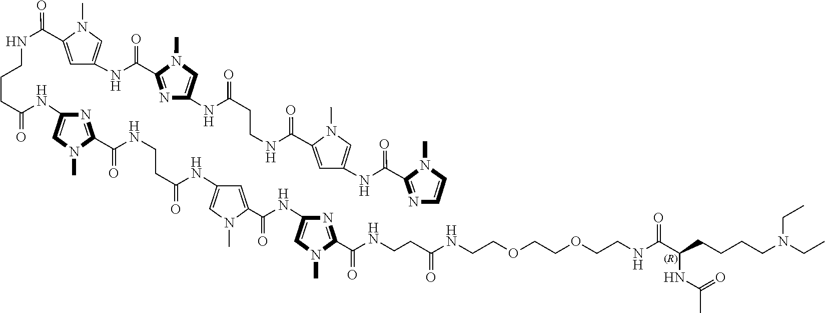 | B-102 |
| 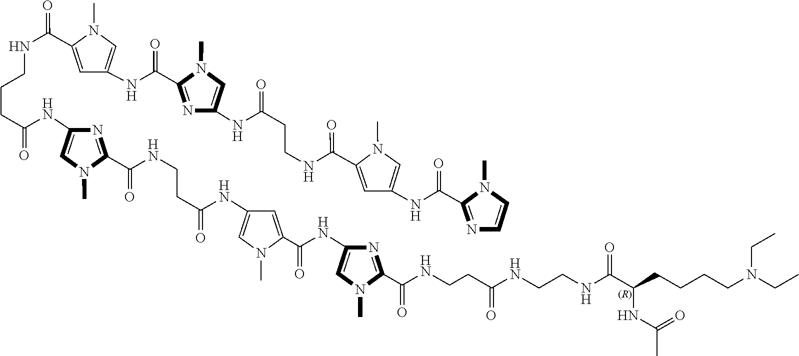 | B-103 |
| 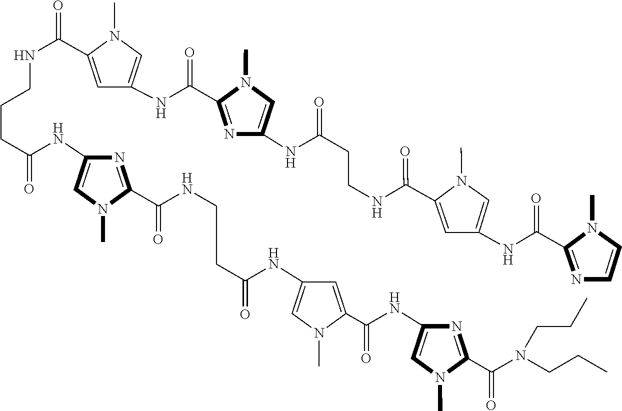 | B-104 |
| 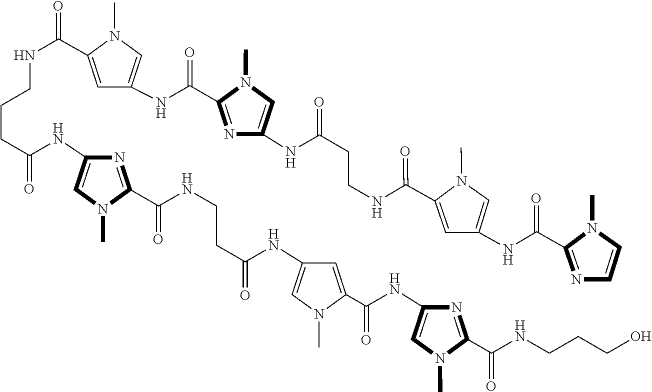 | B-105 |
| 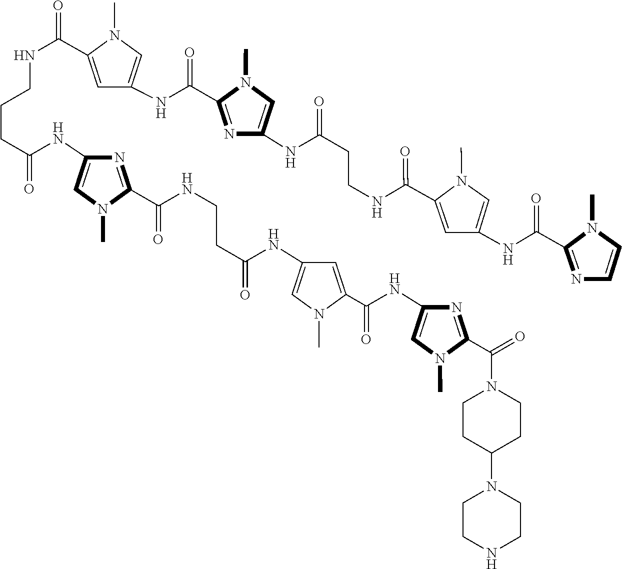 | B-106 |
| 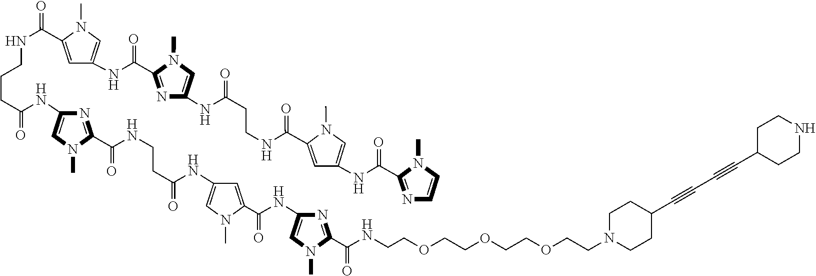 | B-107 |
| 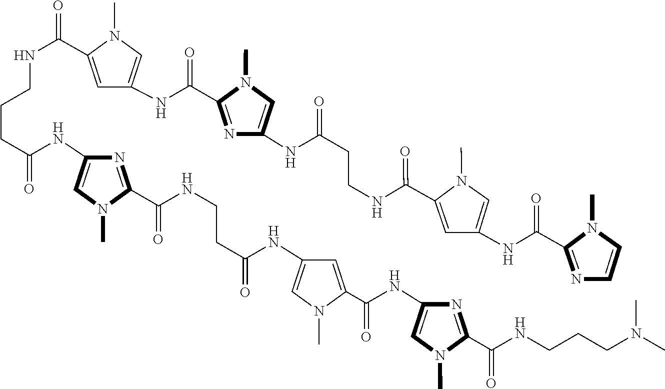 | B-108 |
| 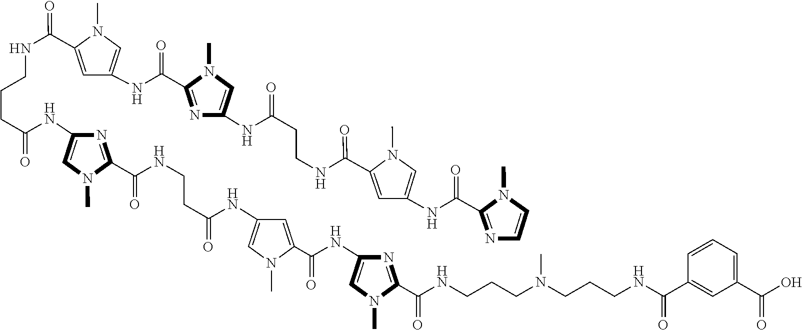 | B-109 |
| 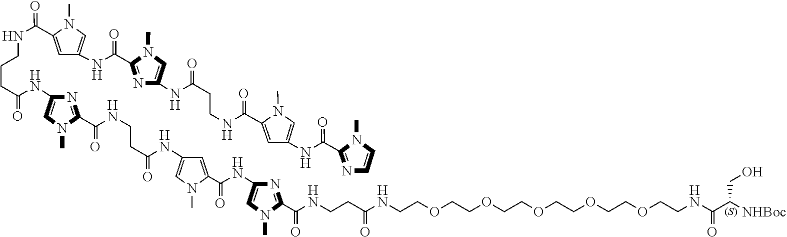 | B-110 |
| 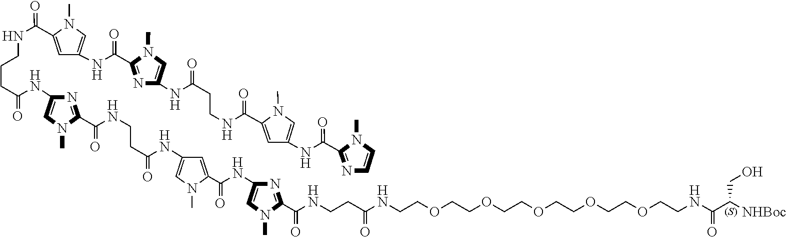 | B-111 |
| 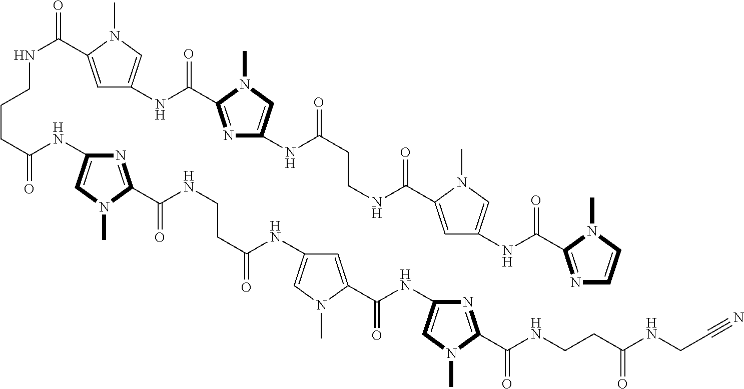 | B-112 |
| 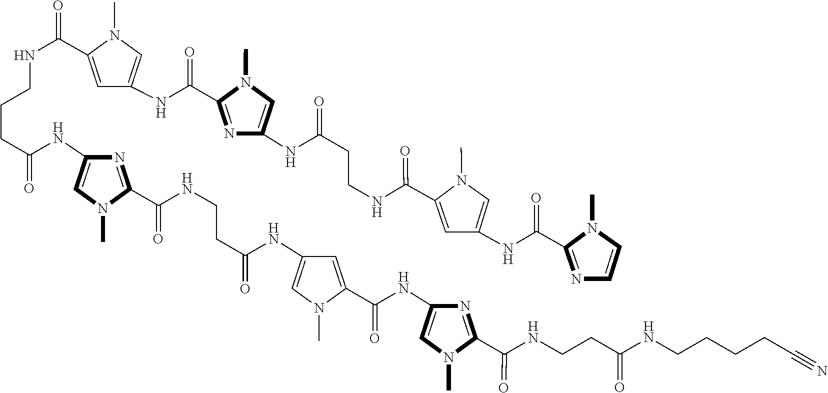 | B-113 |
| 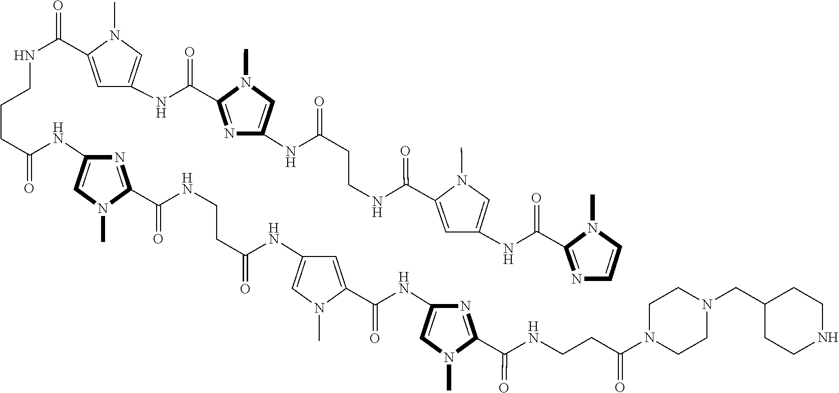 | B-114 |
| 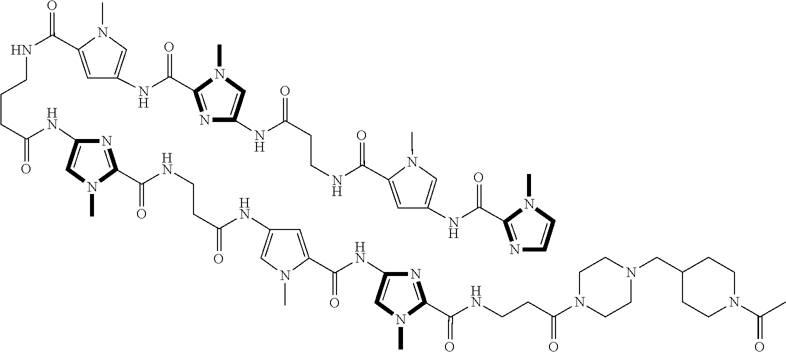 | B-115 |
| 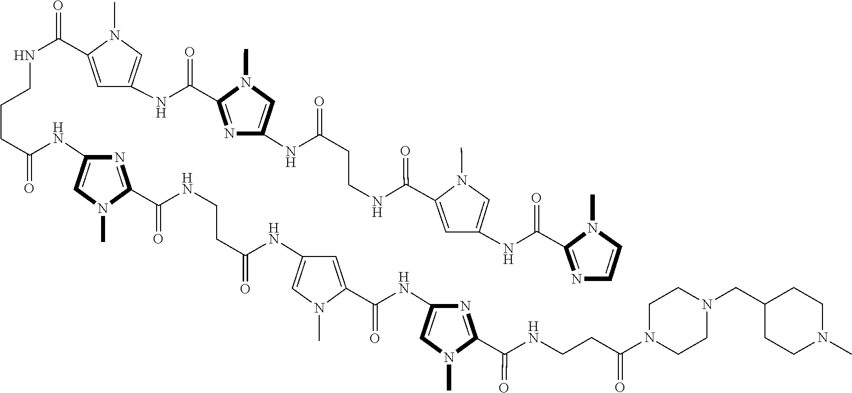 | B-116 |
| 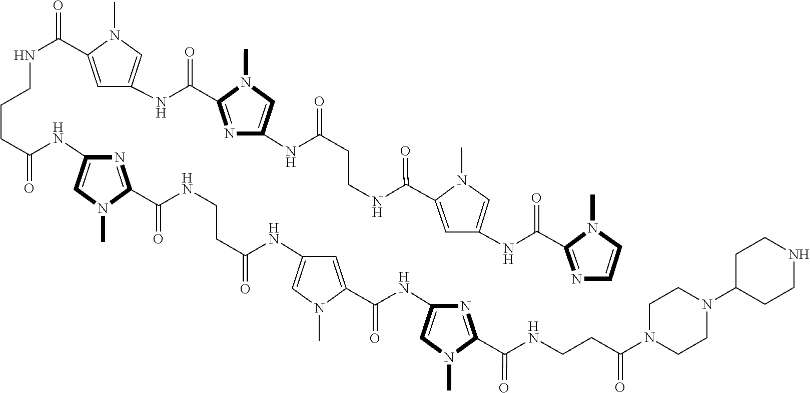 | B-117 |
| 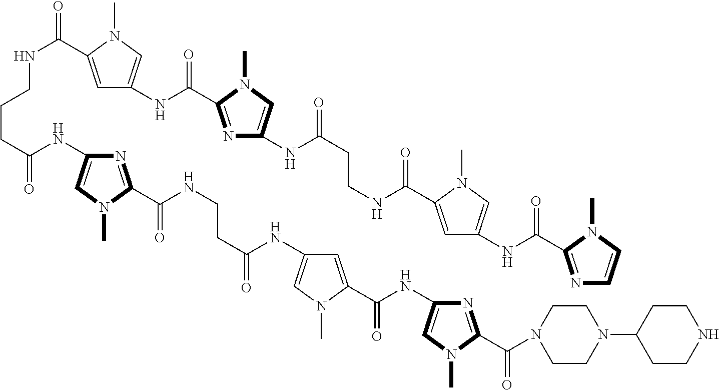 | B-118 |
| 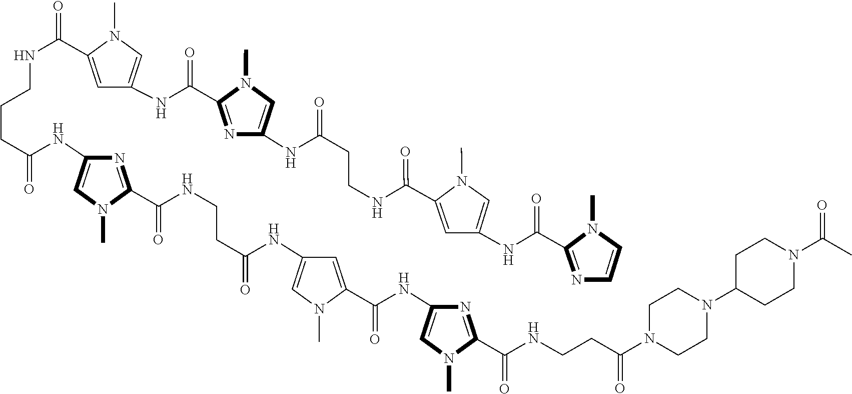 | B-119 |
| 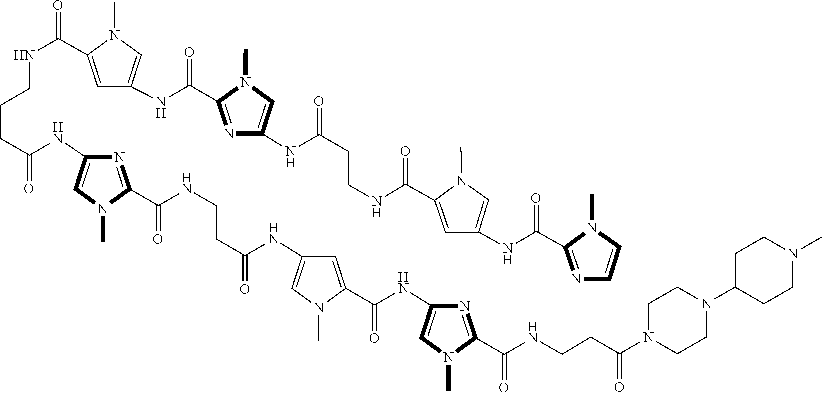 | B-120 |
| 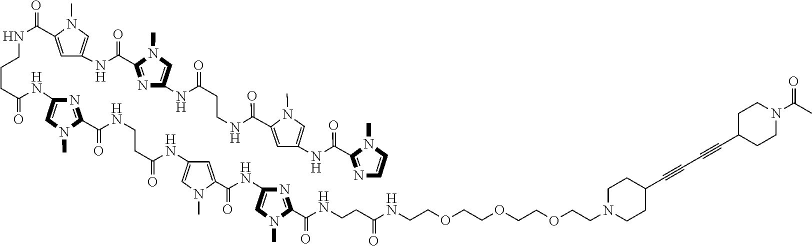 | B-121 |
| 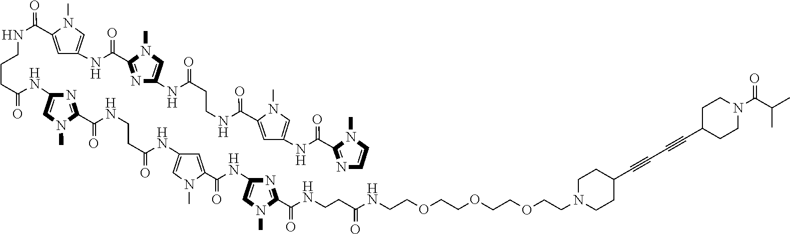 | B-122 |
| 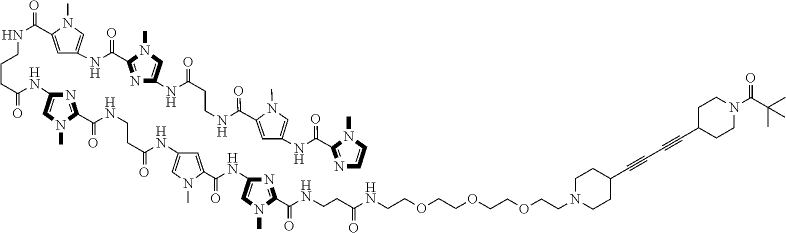 | B-123 |
| 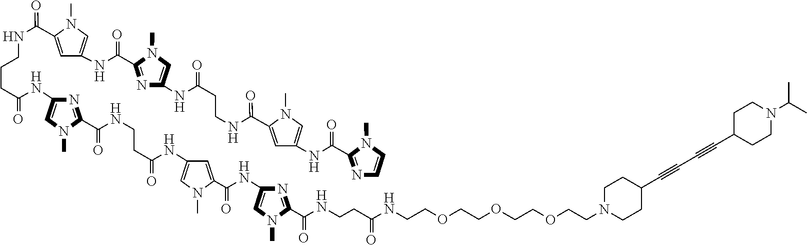 | B-124 |
| 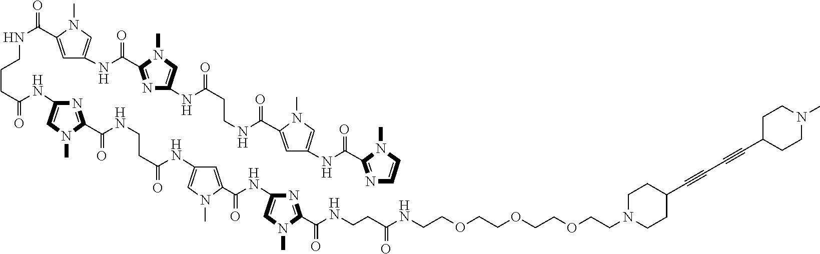 | B-125 |
| 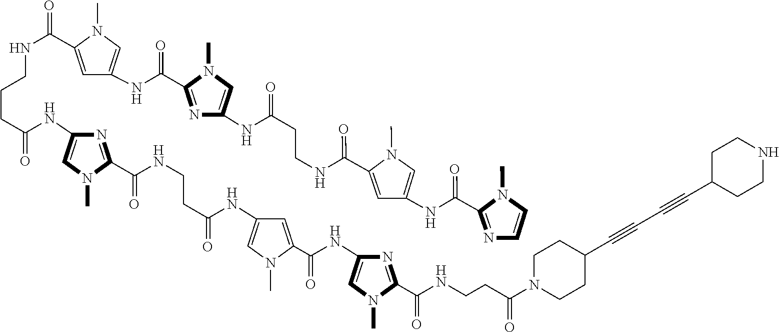 | B-126 |
| 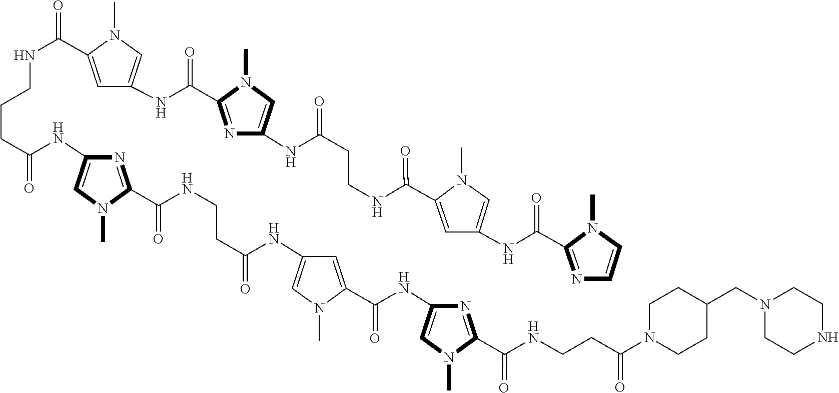 | B-127 |
| 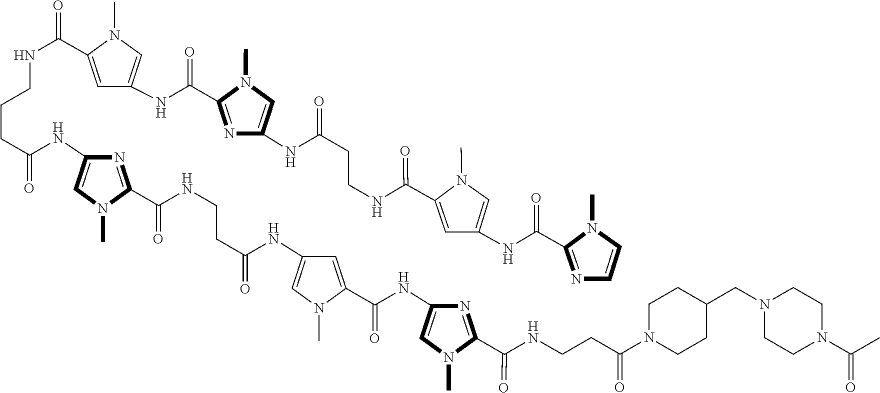 | B-128 |
| 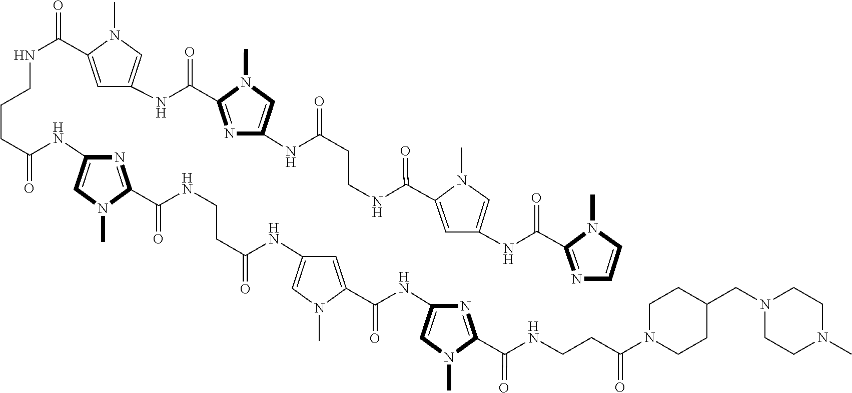 | B-129 |
| 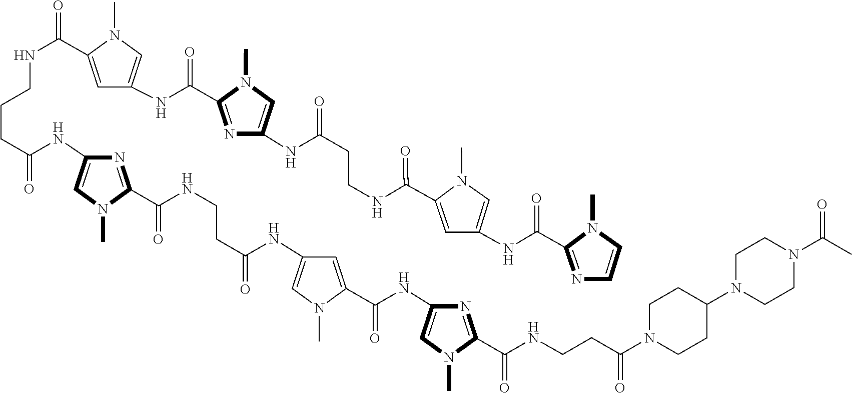 | B-130 |
| 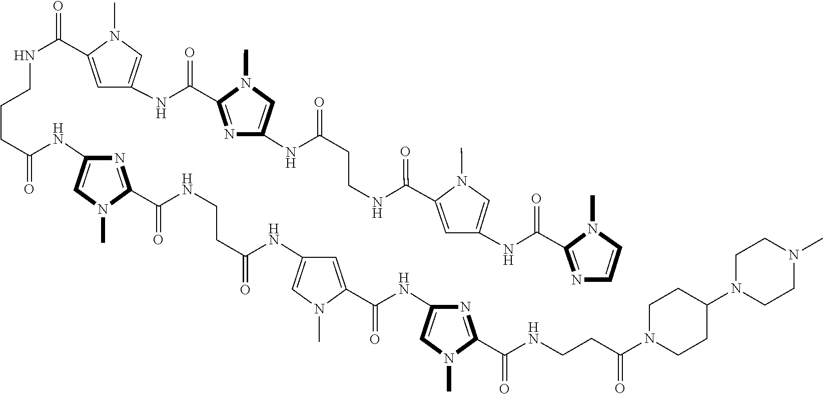 | B-131 |
| 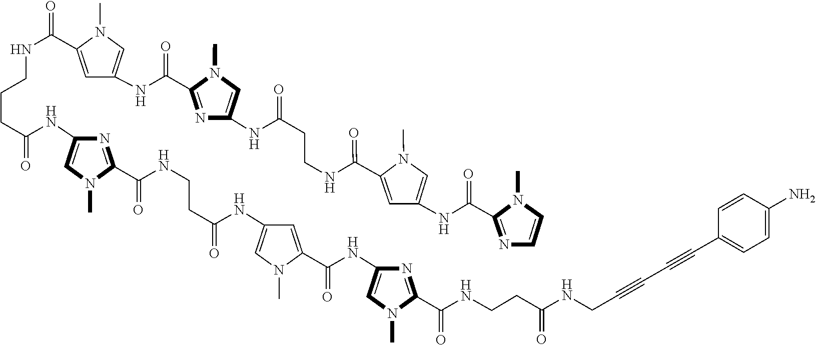 | B-132 |
| 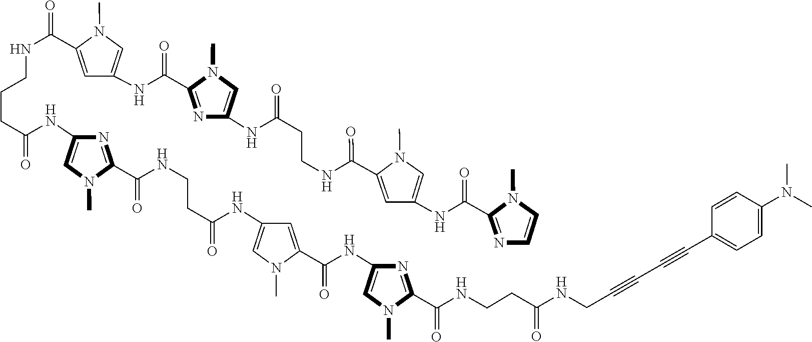 | B-133 |
| 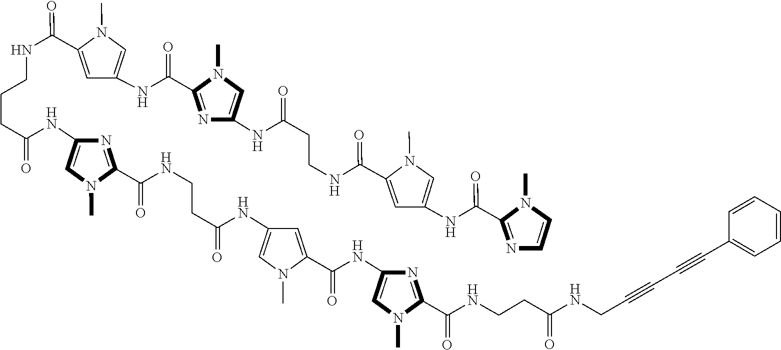 | B-134 |
| 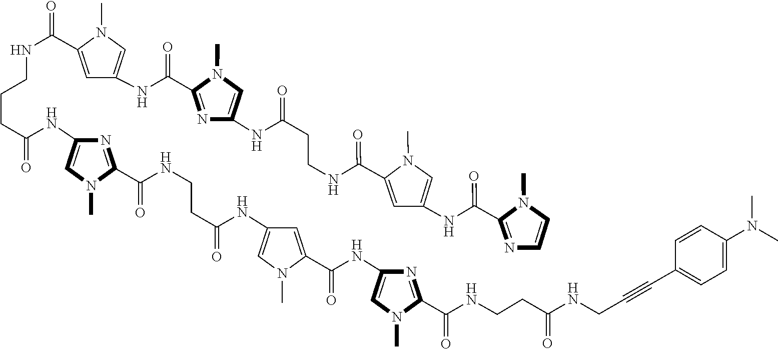 | B-135 |
| 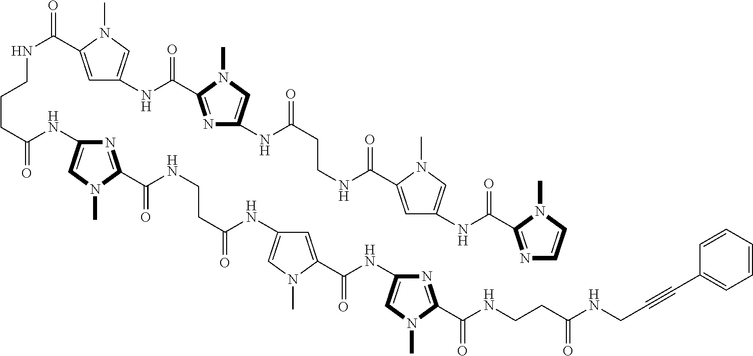 | B-136 |
| 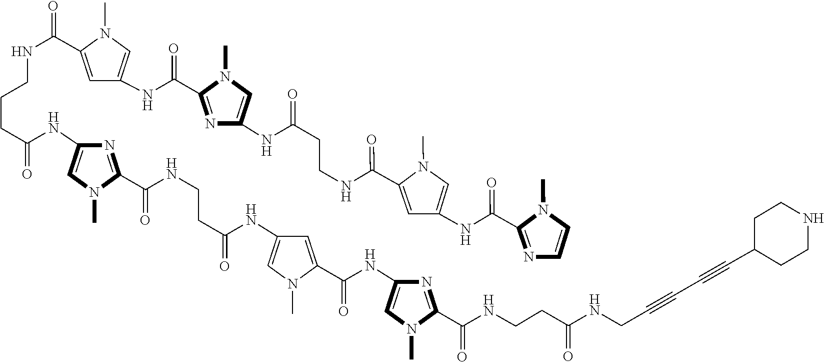 | B-137 |
| 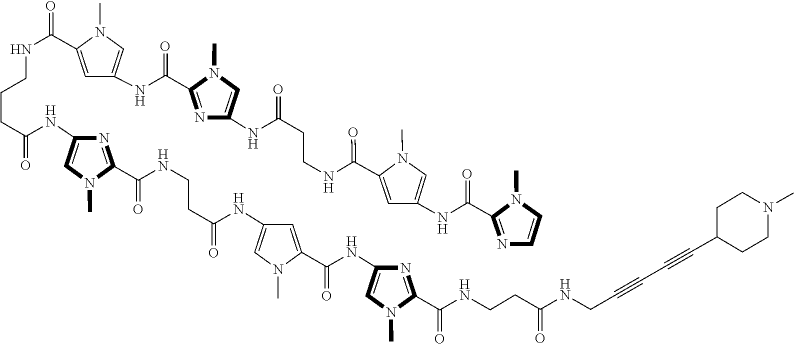 | B-138 |
| 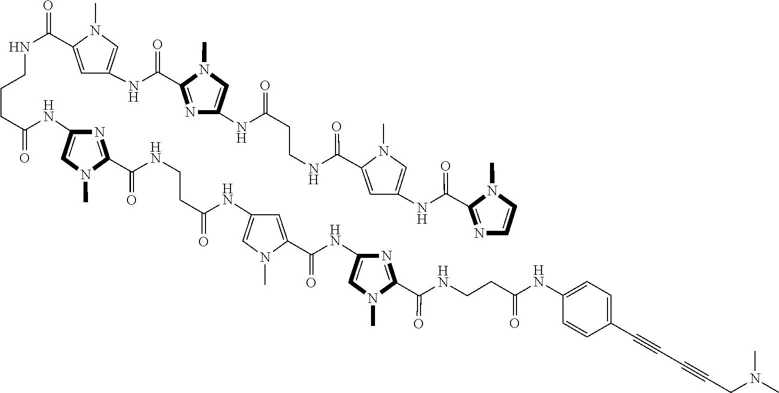 | B-139 |
| 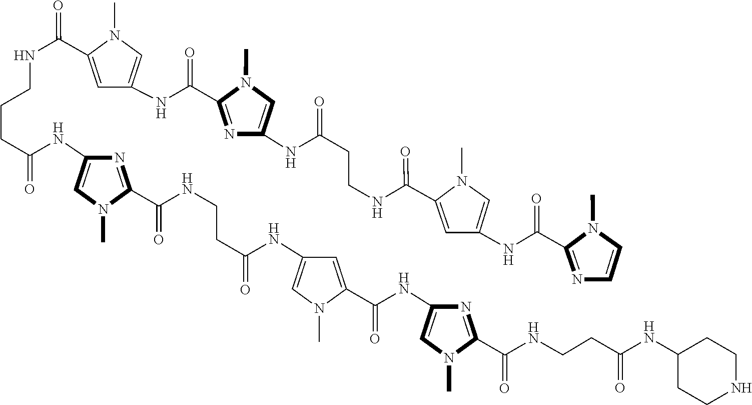 | B-140 |
| 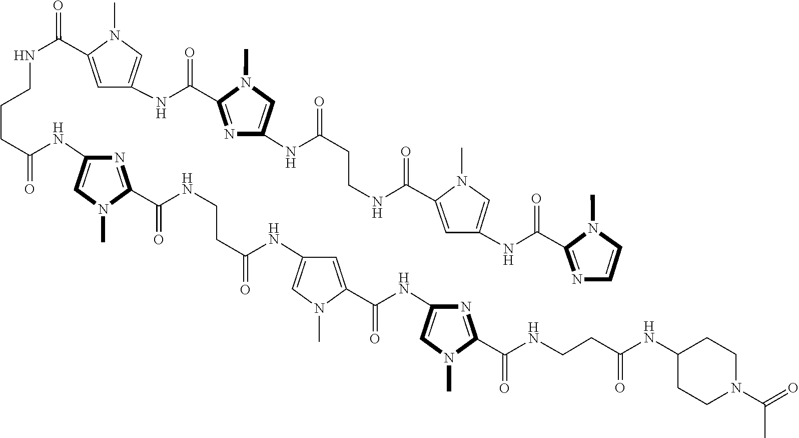 | B-141 |
| 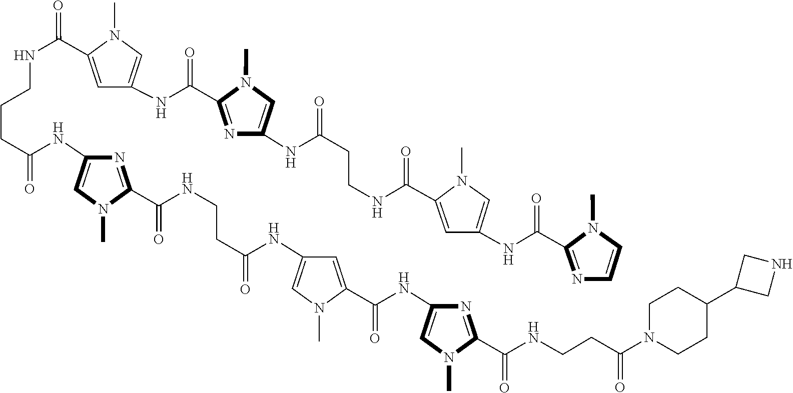 | B-142 |
| 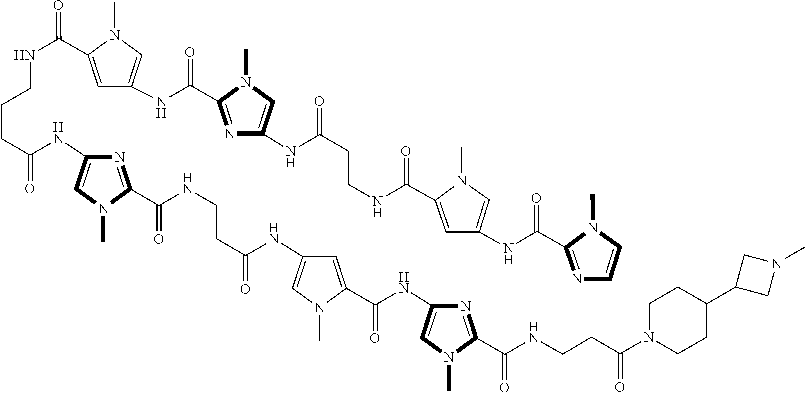 | B-143 |
| 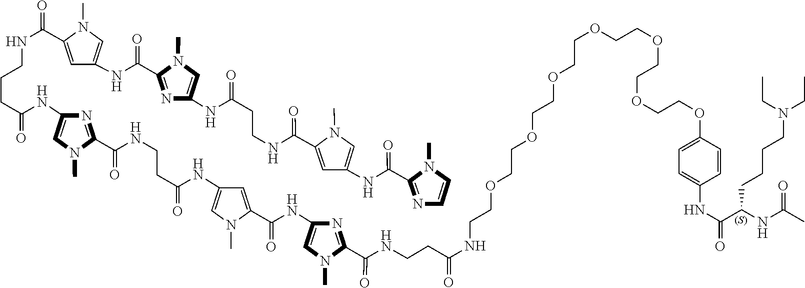 | B-144 |
| 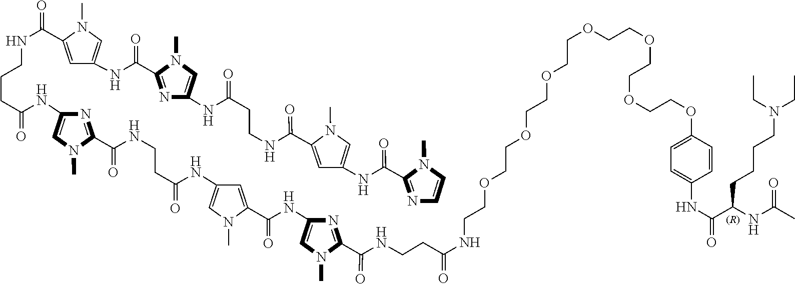 | B-145 |
| 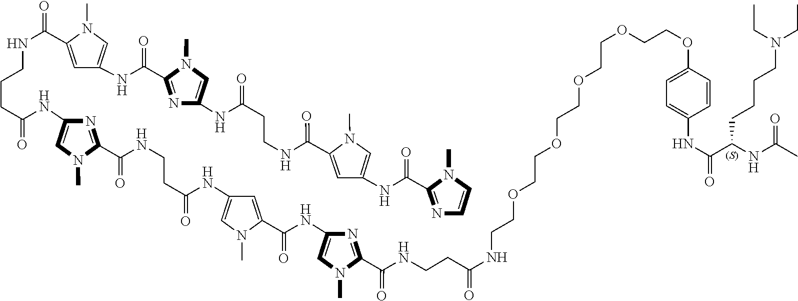 | B-146 |
| 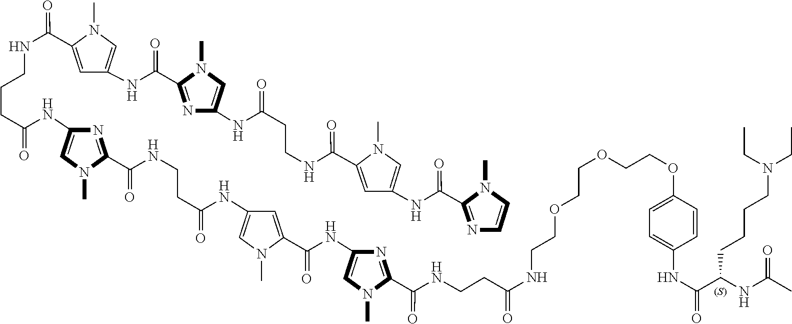 | B-147 |
| 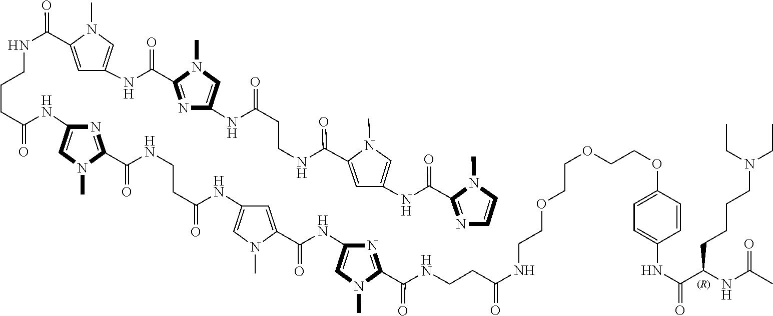 | B-148 |
| 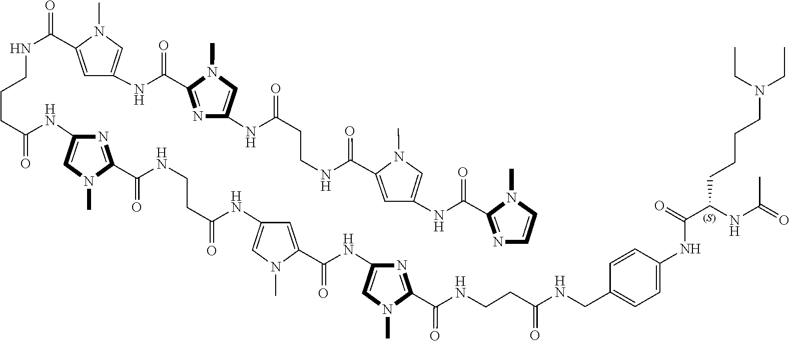 | B-149 |
| 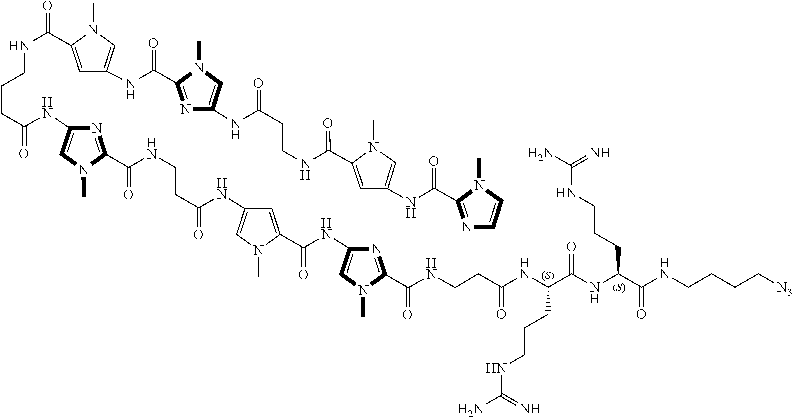 | B-150 |
| 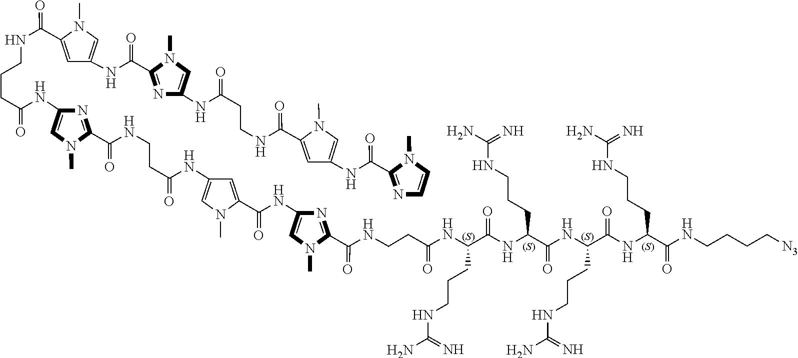 | B-151 |
| 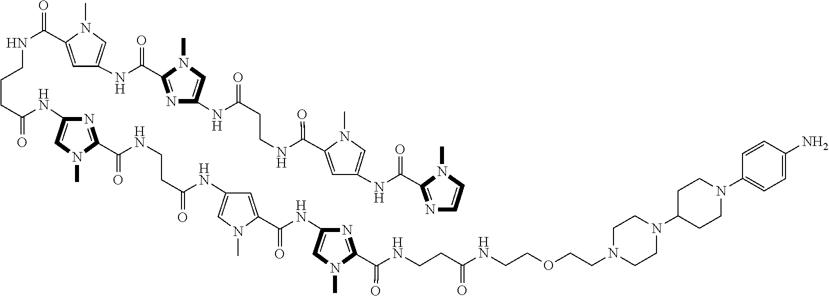 | B-152 |
| 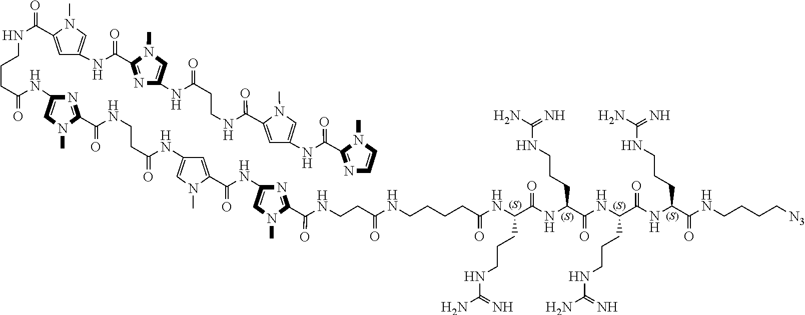 | B-153 |
| 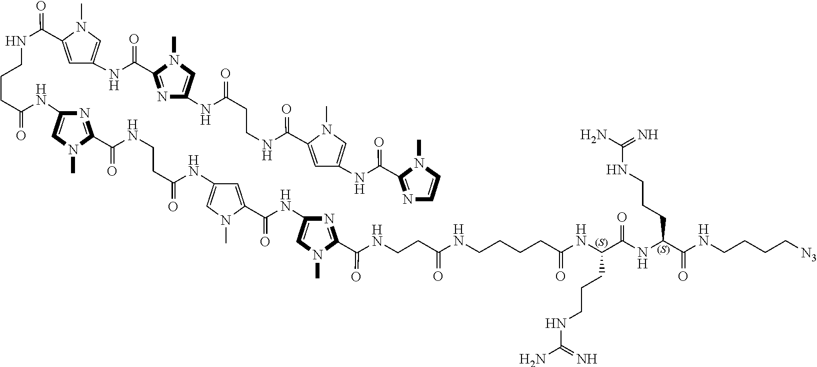 | B-154 |
| 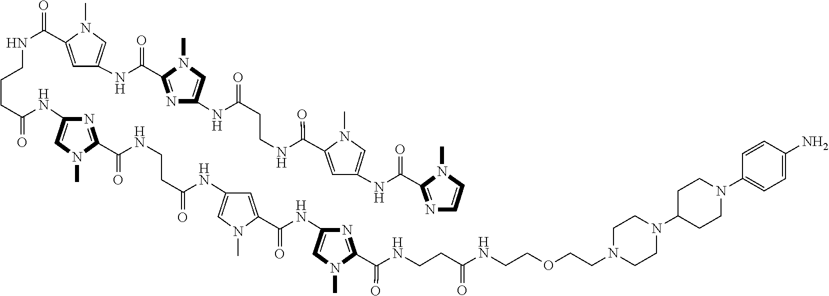 | B-155 |
| 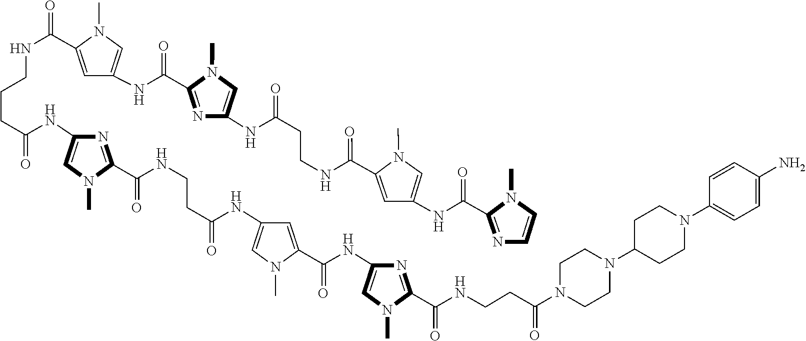 | B-156 |
| 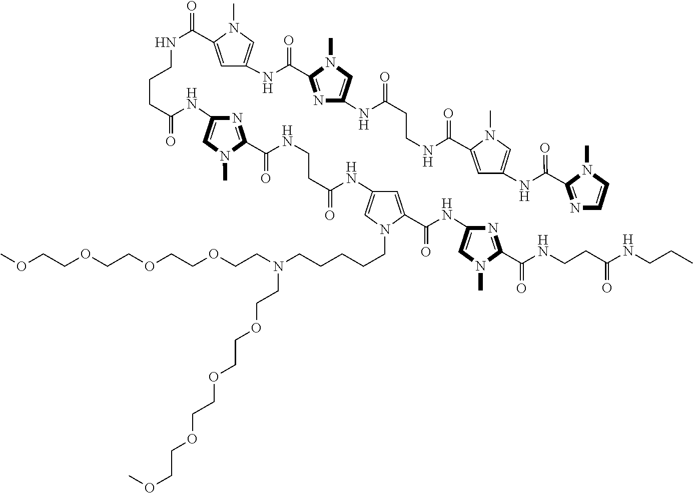 | B-157 |
| 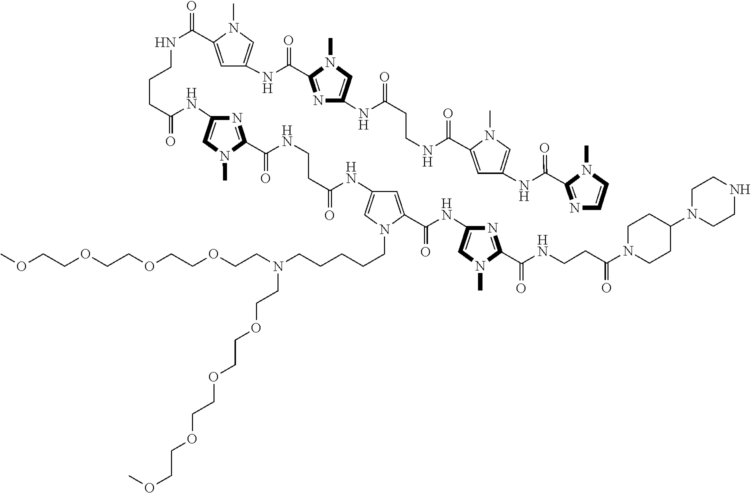 | B-158 |
| 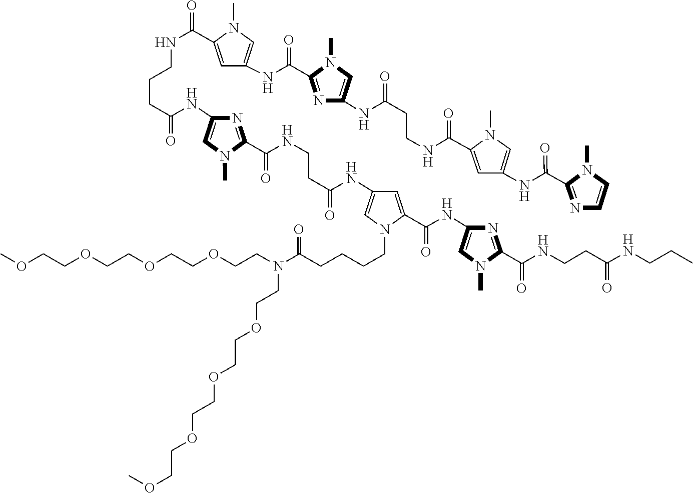 | B-159 |
| 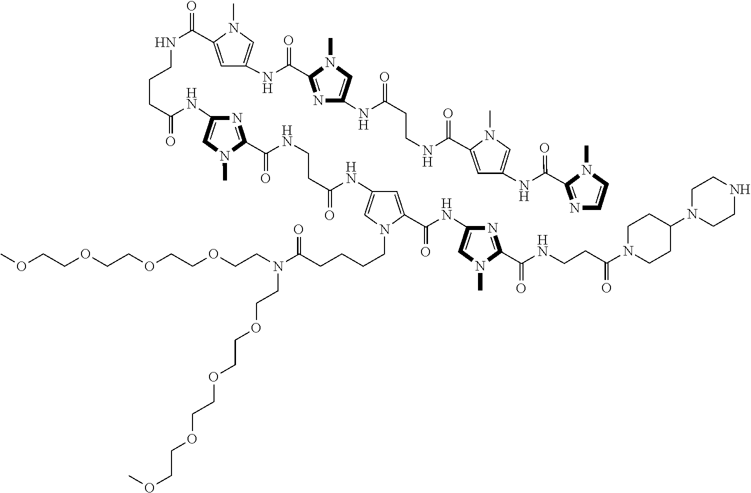 | 160 |
| 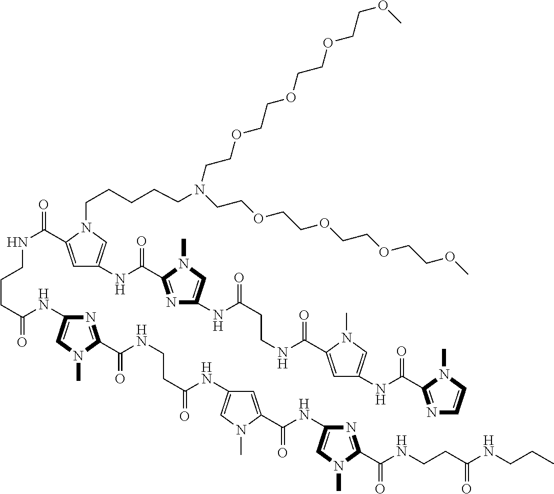 | B-161 |
| 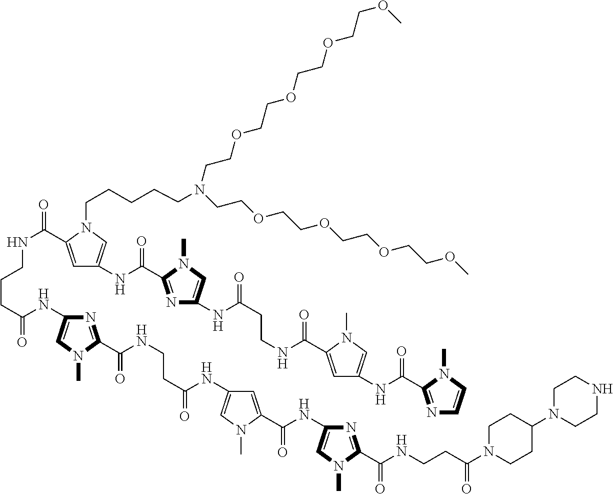 | B-162 |
| 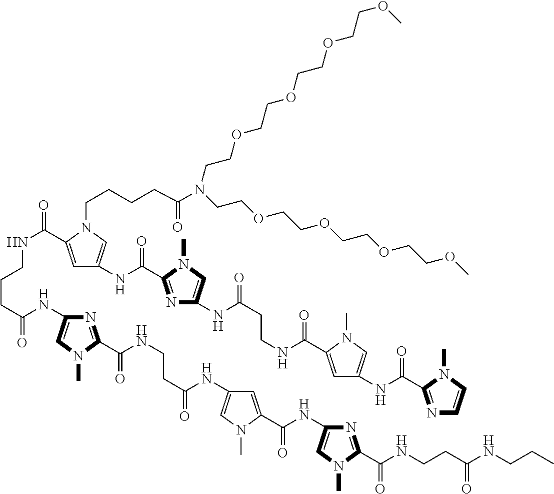 | B-163 |
| 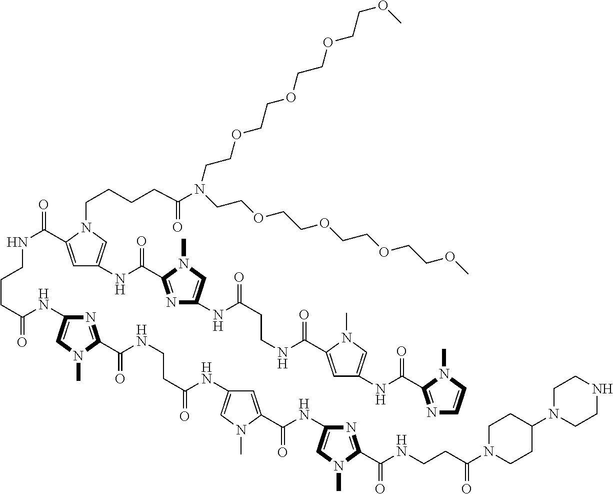 | B-164 |
| 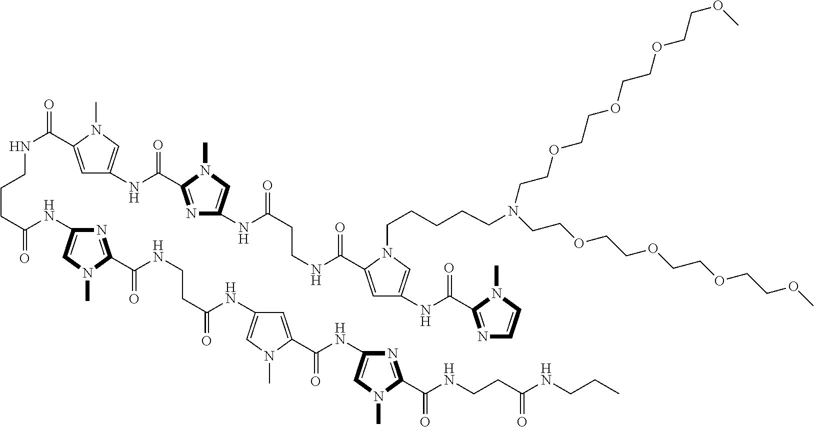 | B-165 |
| 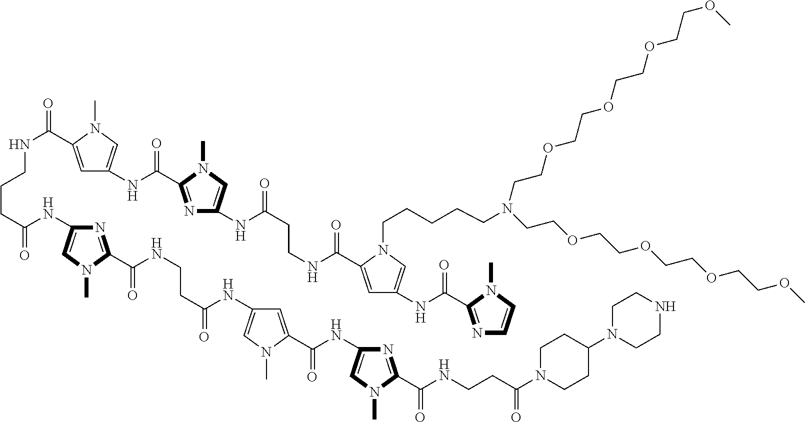 | B-166 |
| 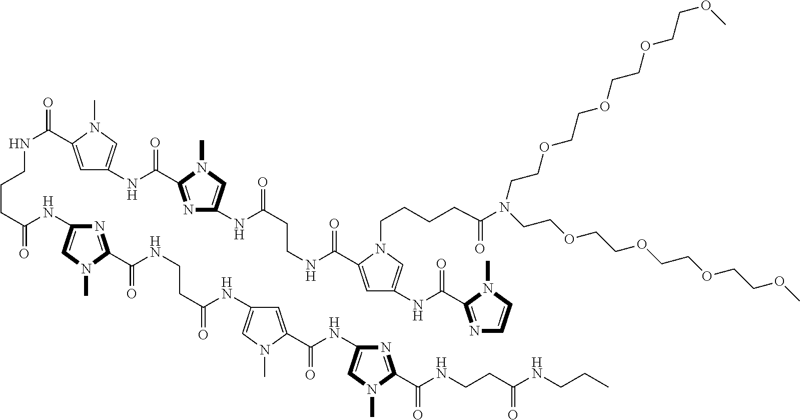 | B-167 |
| 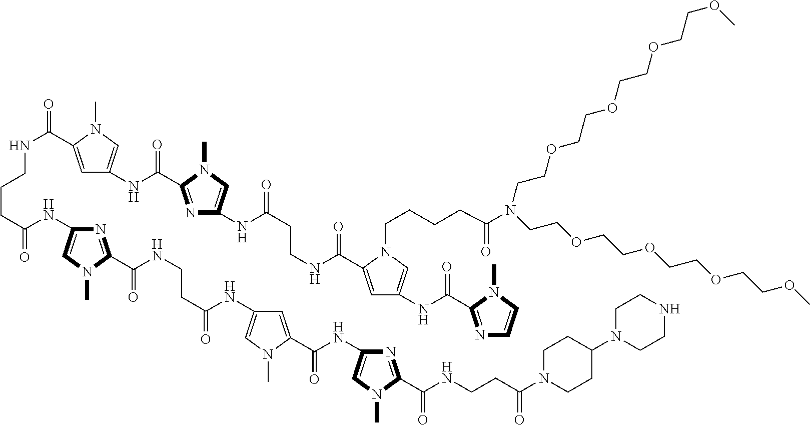 | B-168 |
| 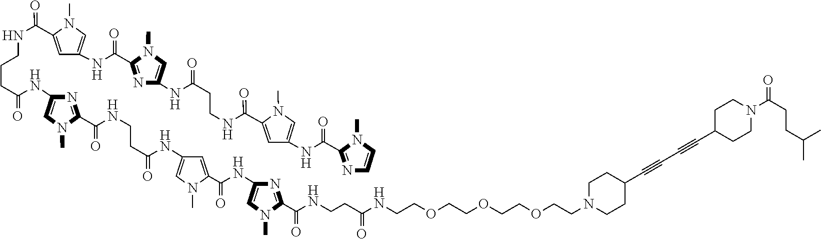 | B-169 |
| 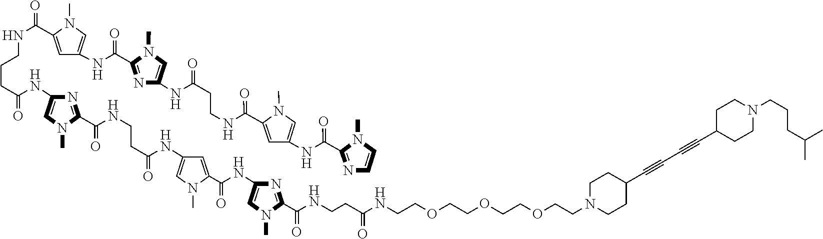 | B-170 |
| 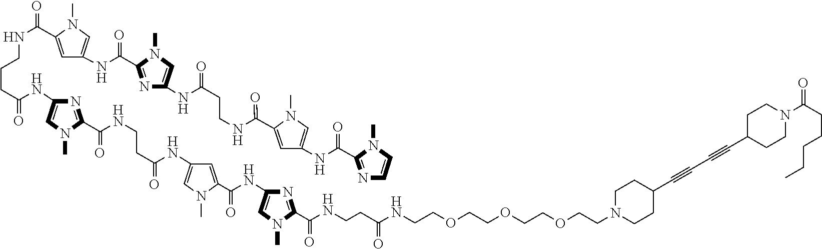 | B-171 |
| 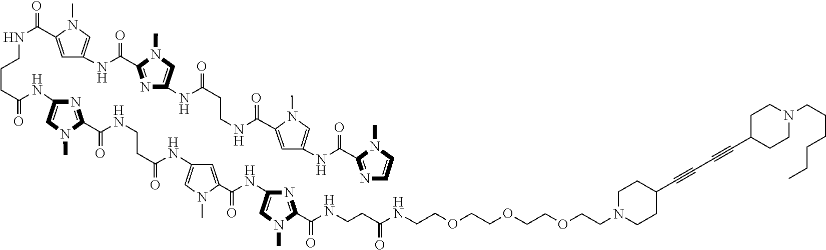 | B-172 |
| 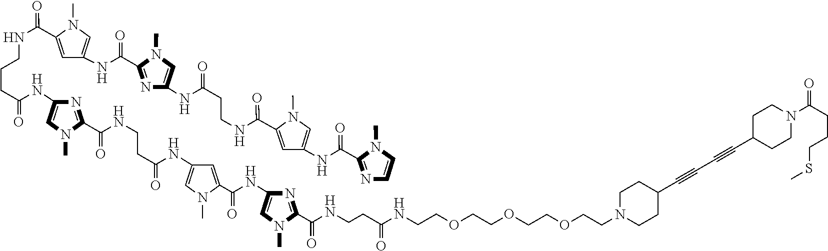 | B-173 |
| 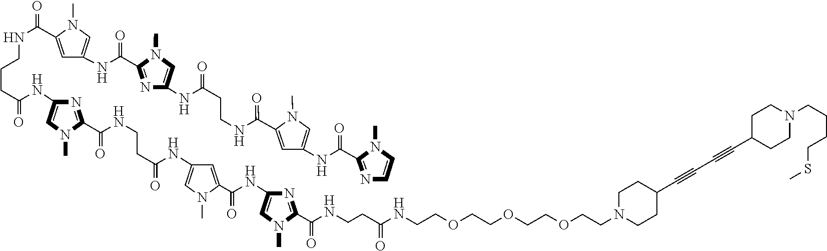 | B-174 |
| 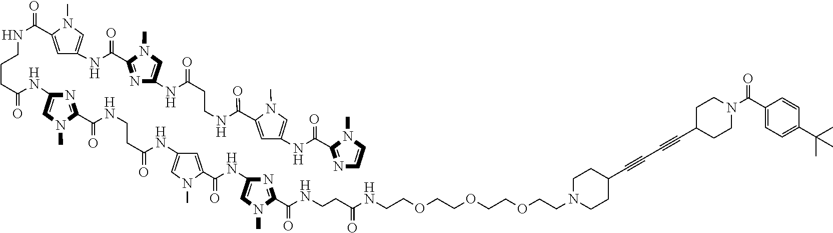 | B-175 |
| 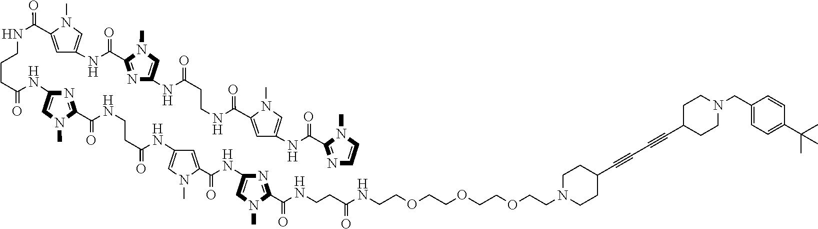 | B-176 |
| 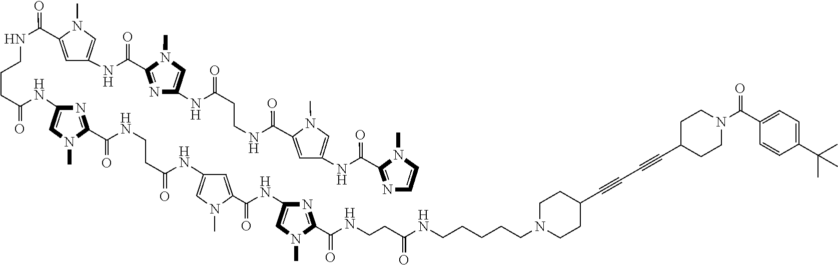 | B-177 |
| 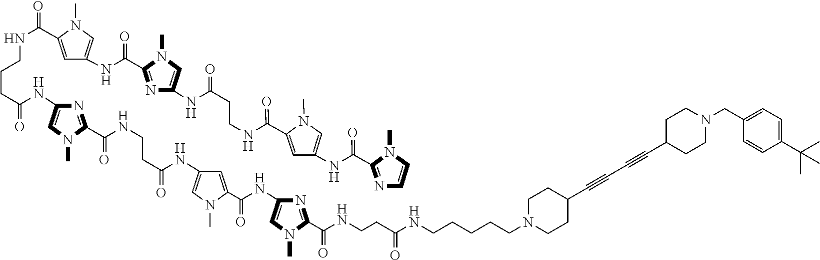 | B-178 |
| 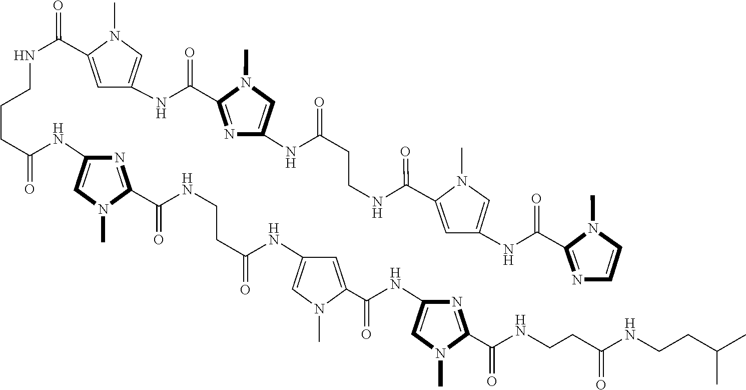 | B-179 |
| 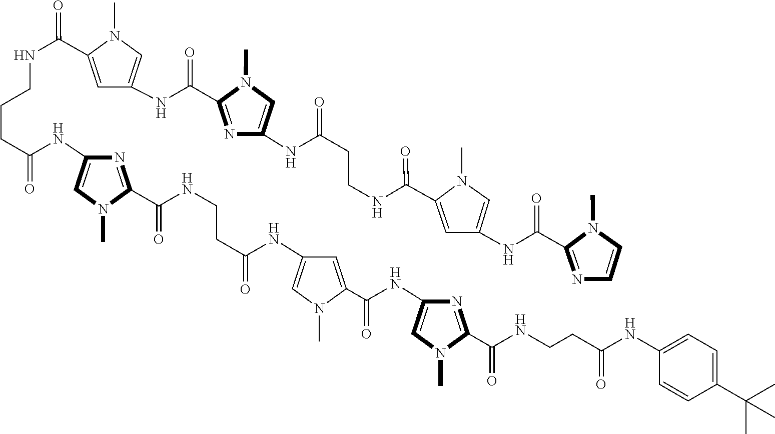 | B-180 |
| 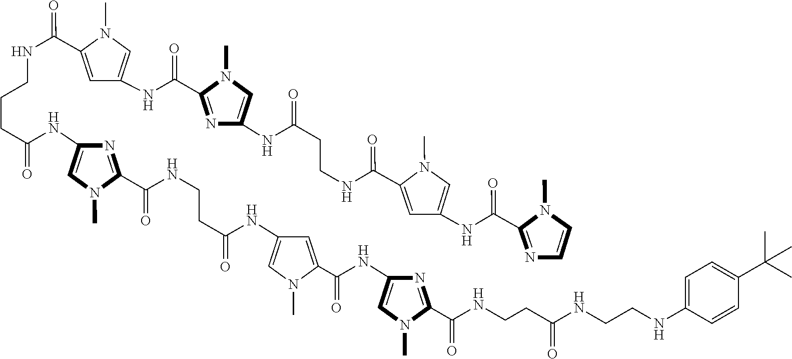 | B-181 |
| 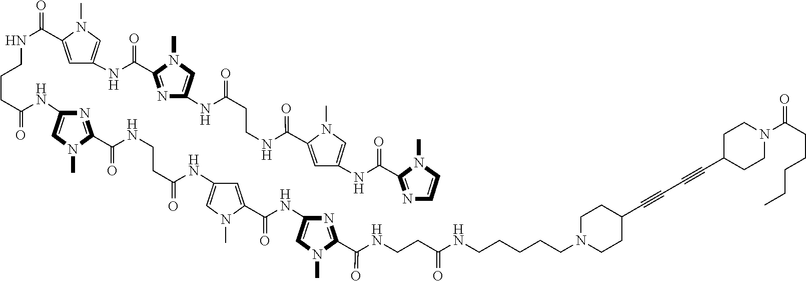 | B-182 |
| 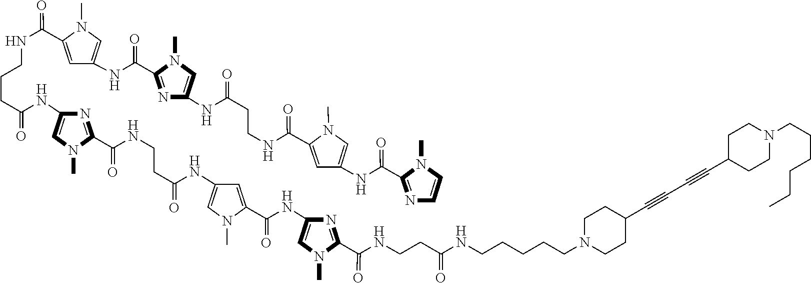 | B-183 |
| 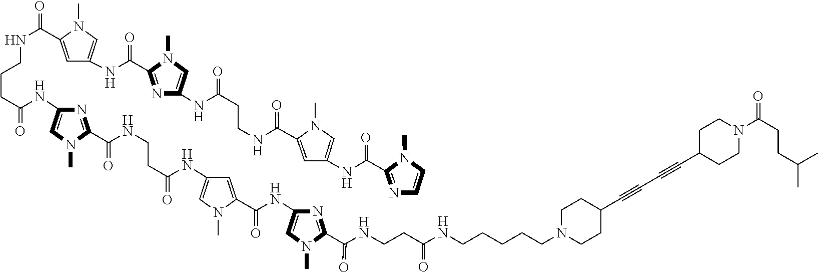 | B-184 |
| 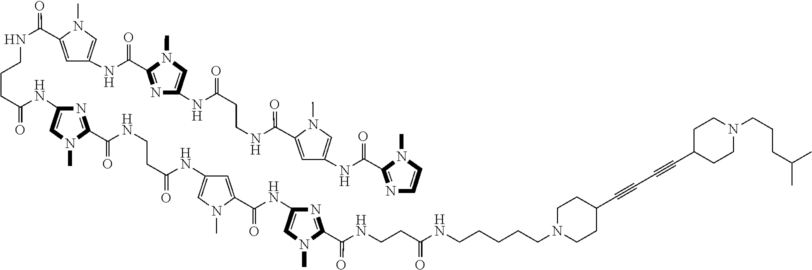 | B-185 |
| 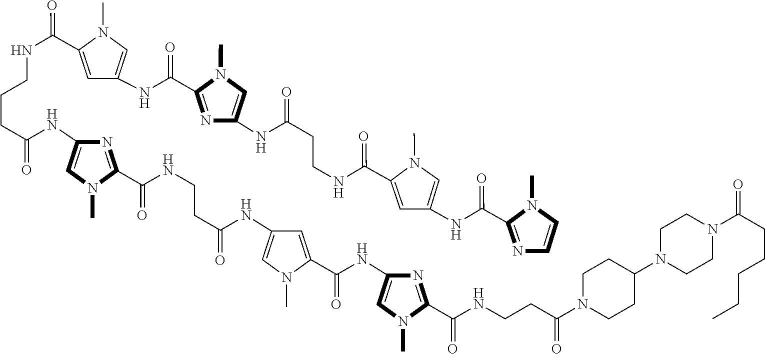 | B-186 |
| 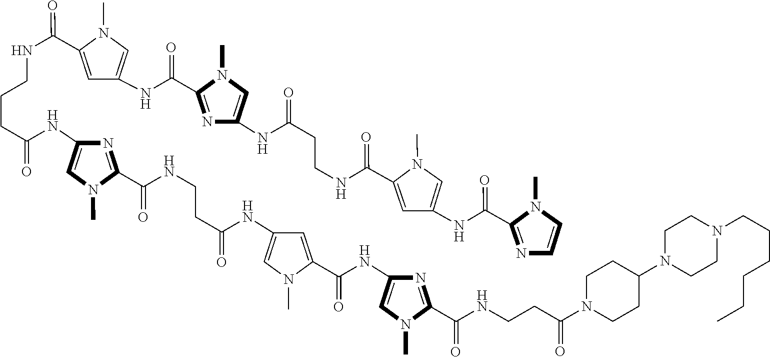 | B-187 |
| 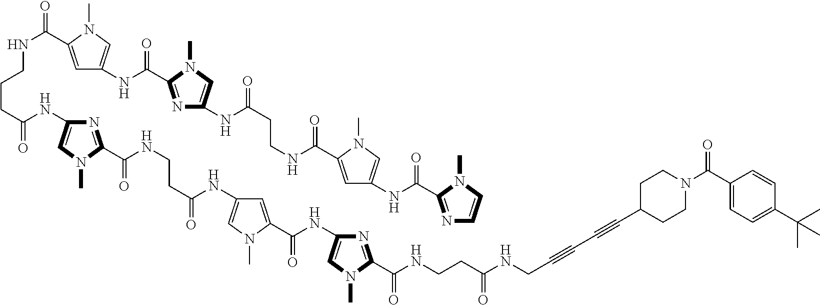 | B-188 |
| 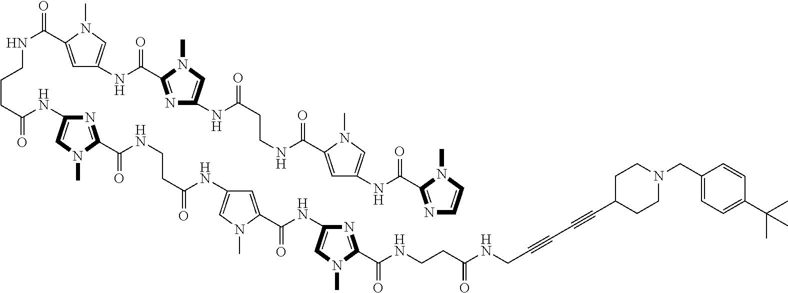 | B-189 |
| 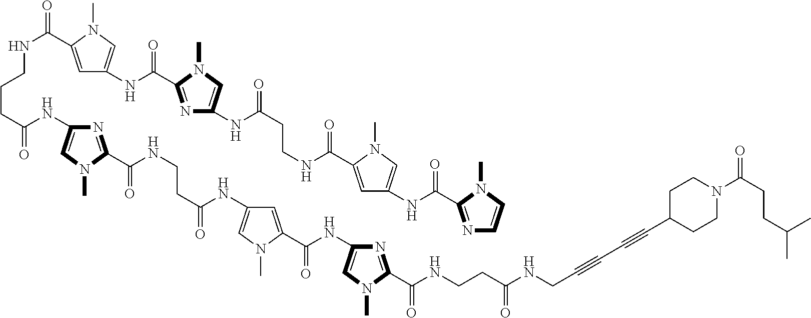 | B-190 |
| 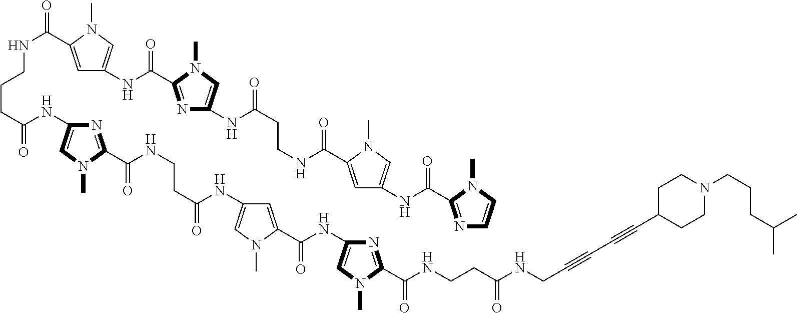 | B-191 |
| 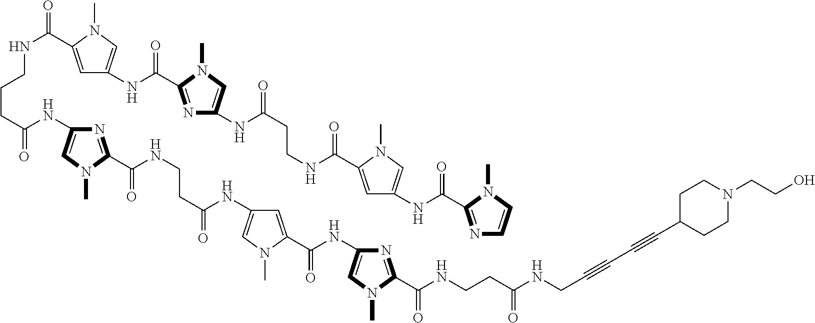 | B-192 |
| 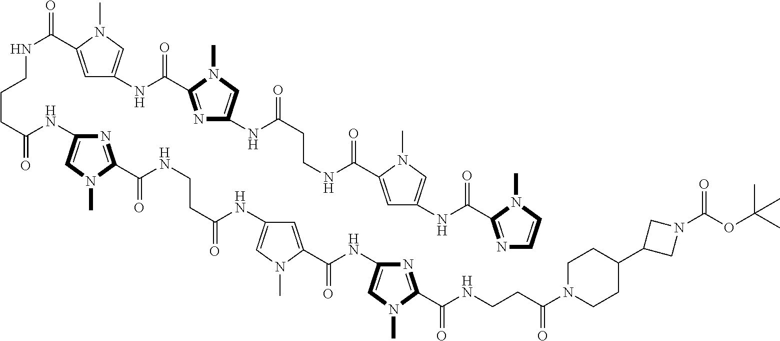 | B-193 |
| 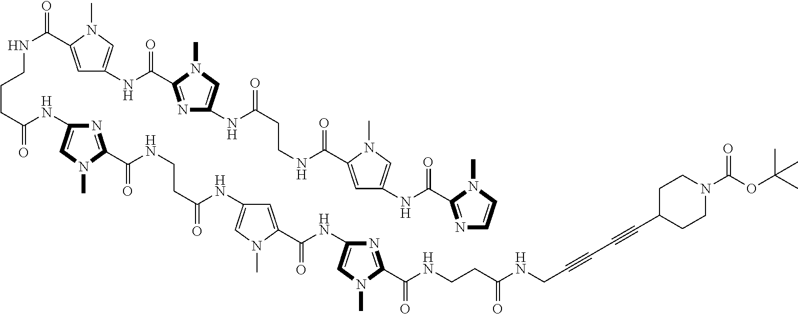 | B-194 |
| 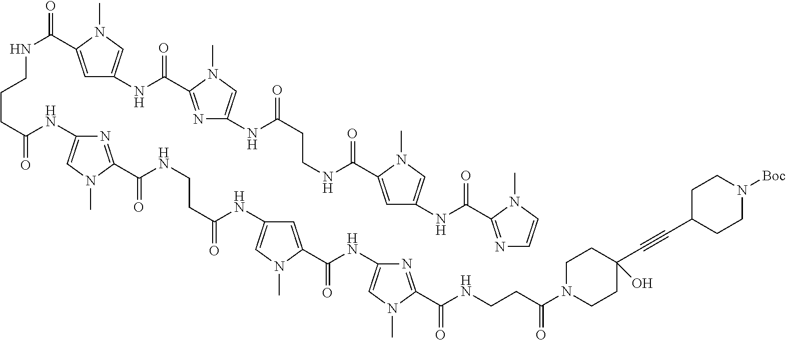 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)N4CCC(O)(C#CC5CCN(C(=O)OC(C)(C)C)CC5)CC4)n3)n(C)c2)n1] | B-195 |
| 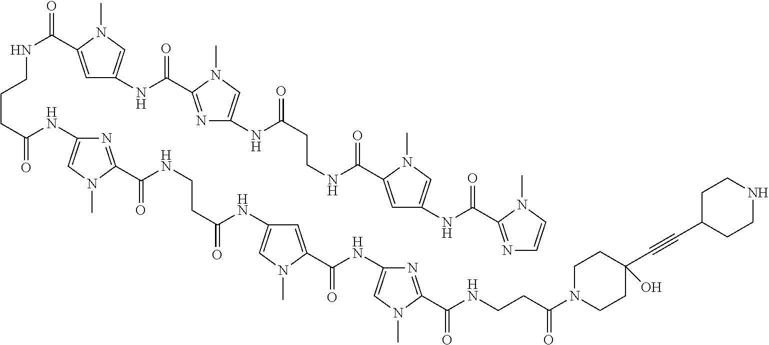 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)N4CCC(O)(C#CC5CCNCC5)CC4)n3)n(C)c2)n1] | B-196 |
| 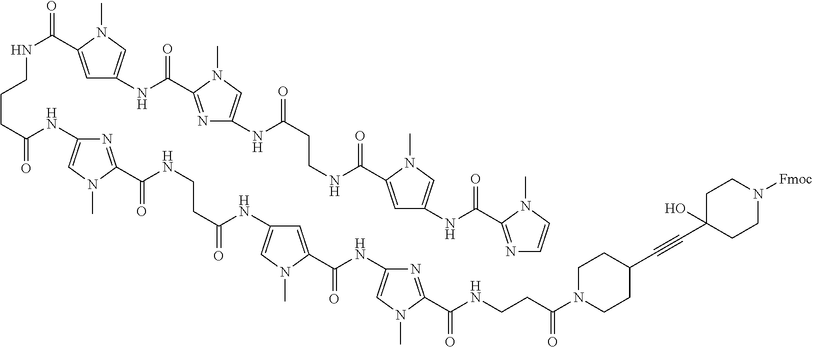 | B-197 |
| 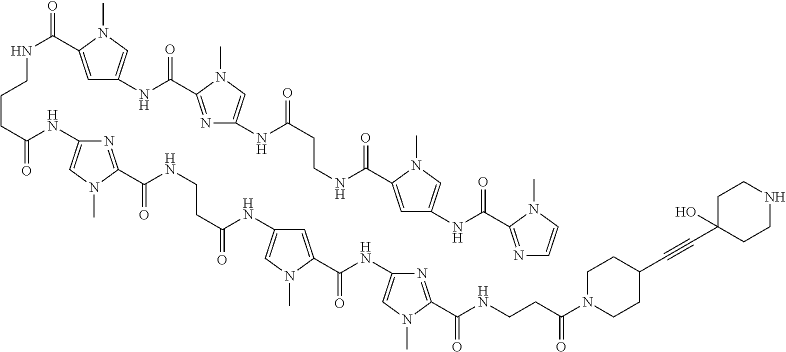 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)N4CCC(C#CC5(O)CCNCC5)CC4)n3)n(C)c2)n1] | B-198 |
| 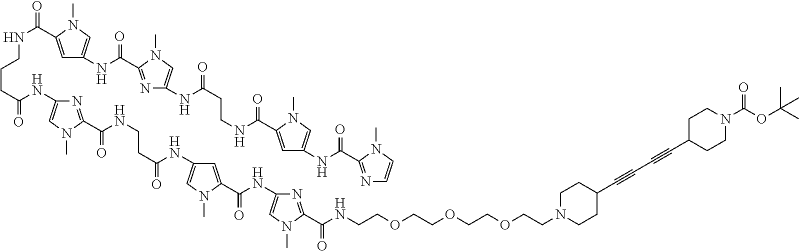 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCOCCOCCOCCN4CCC(C#CC#CC5CCN(C(=O)OC(C)(C)C)CC5)CC4)n3)n(C)c2)n1] | B-199 |
| 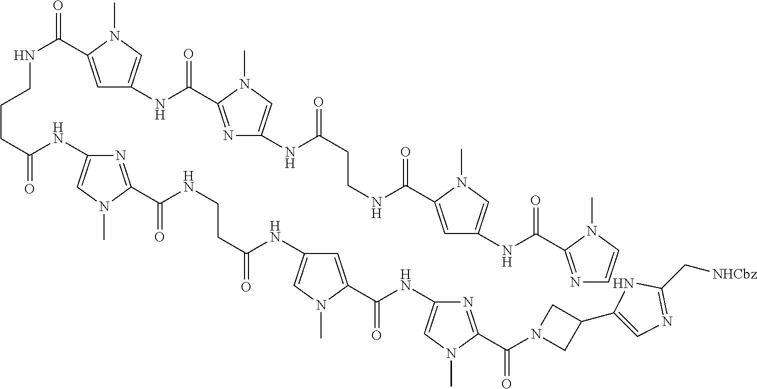 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)N4CC(c5cnc(CNC(=O)OCc6ccccc6)[nH]5)C4)n3)n(C)c2)n1] | B-201 |
| 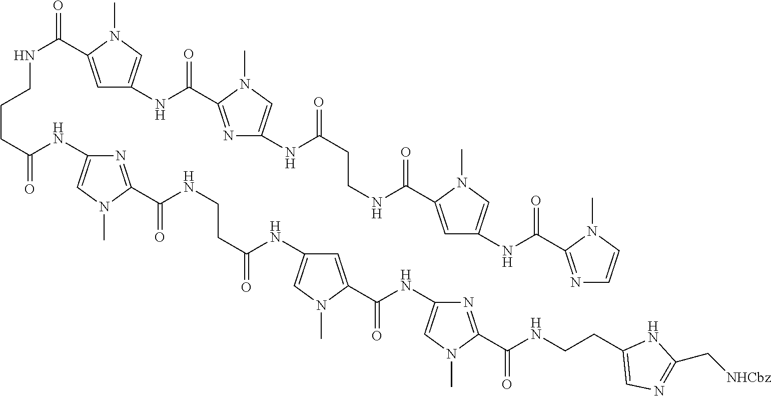 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCc4cnc(CNC(=O)OCc5ccccc5)[nH]4)n3)n(C)c2)n1] | B-202 |
| 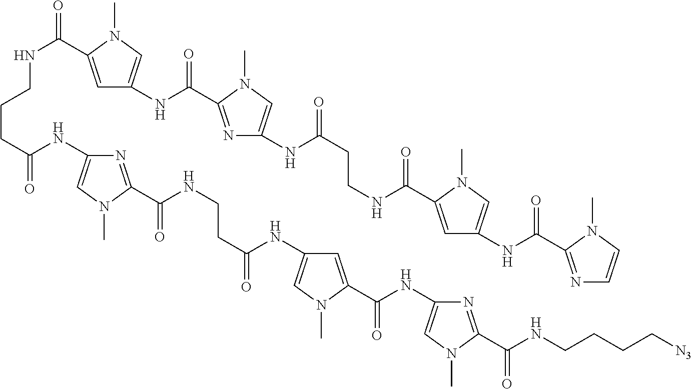 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCCCN=[N+]=[N-])n3)n(C)c2)n1] | B-203 |
| 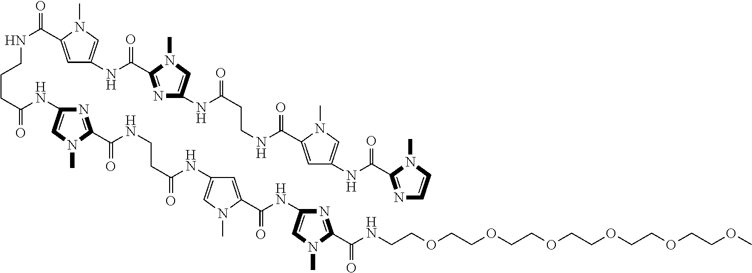 | B-204 |
| 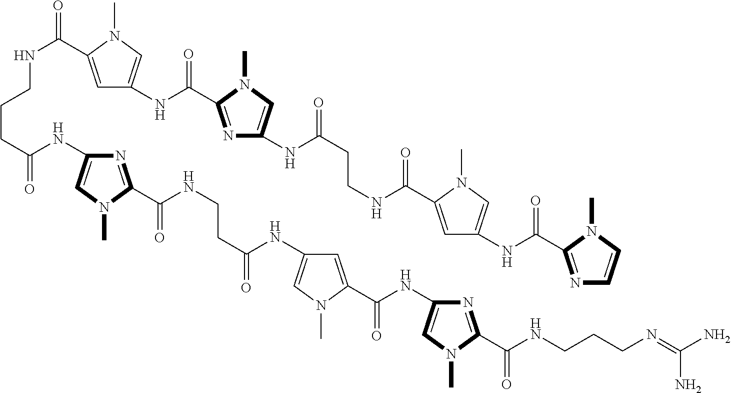 | B-205 |
| 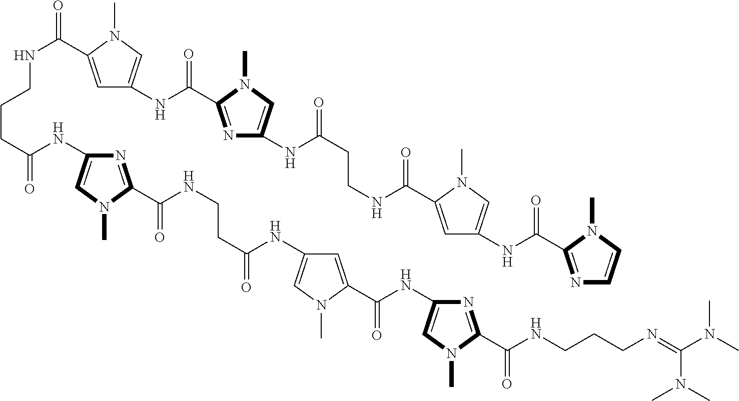 | B-206 |
| 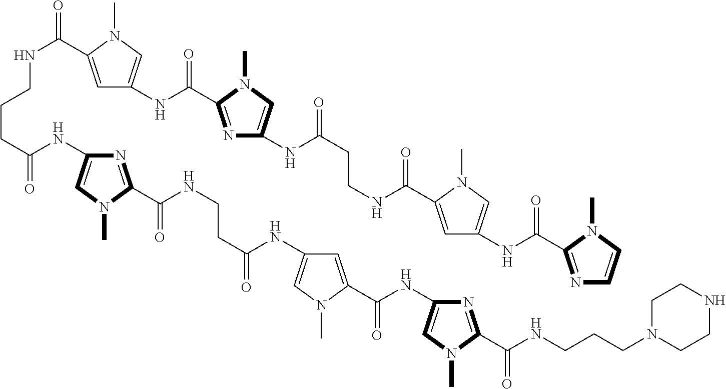 | B-207 |
| 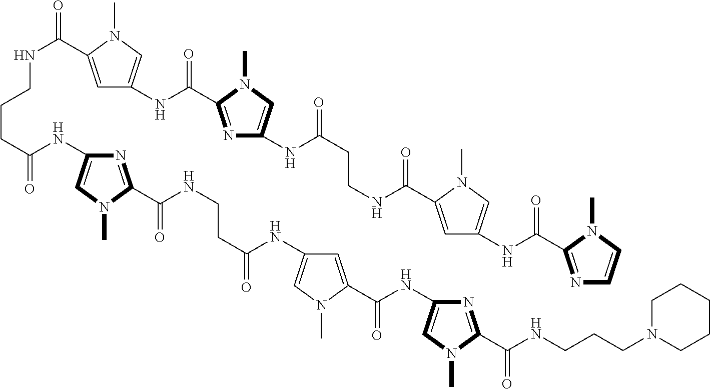 | B-208 |
| 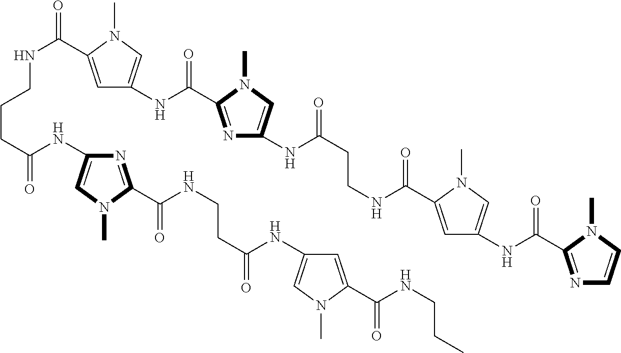 | B-209 |
| 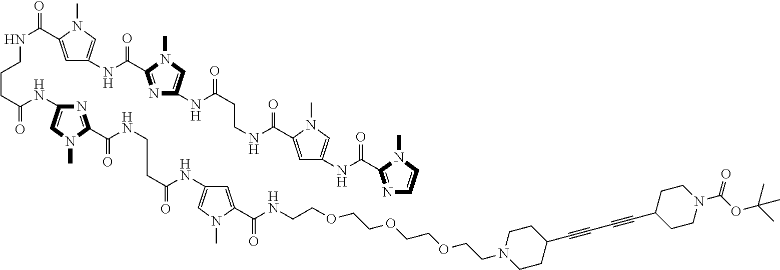 | B-210 |
| 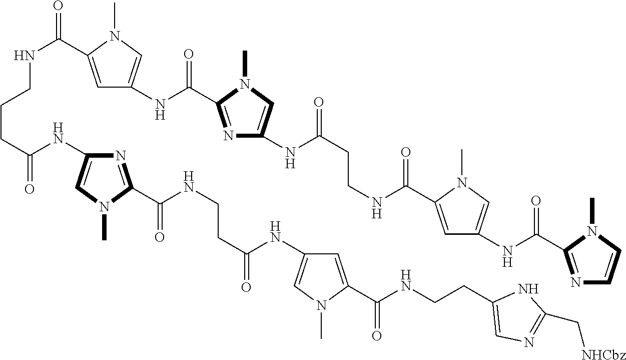 | B-211 |
| 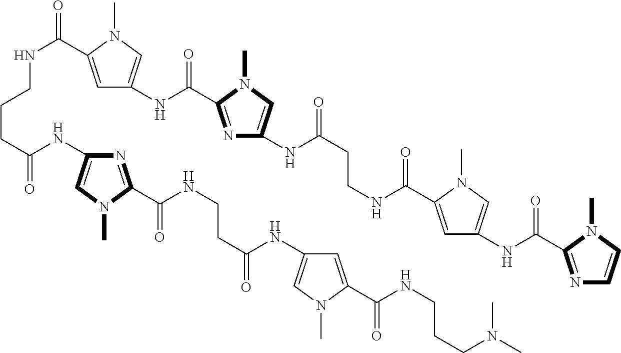 | B-212 |
| 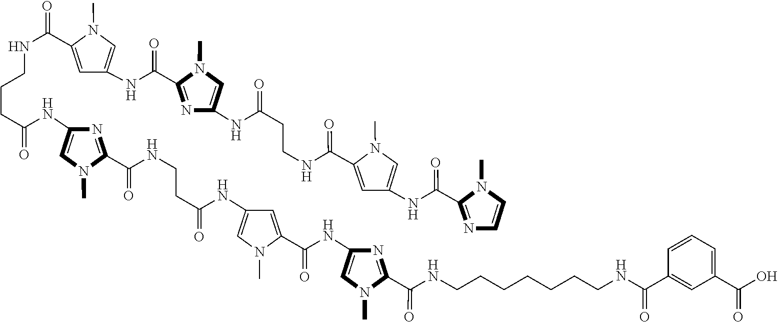 | B-213 |
| 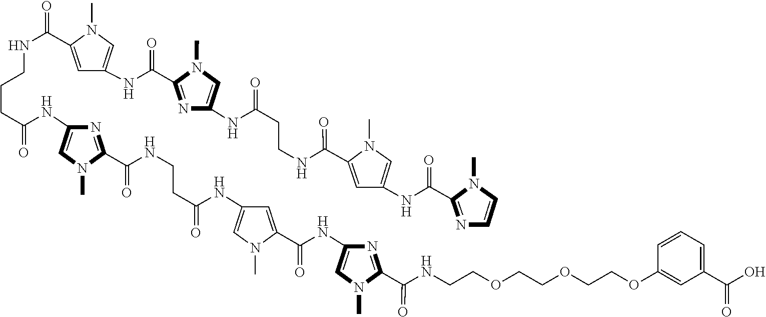 | B-214 |
| 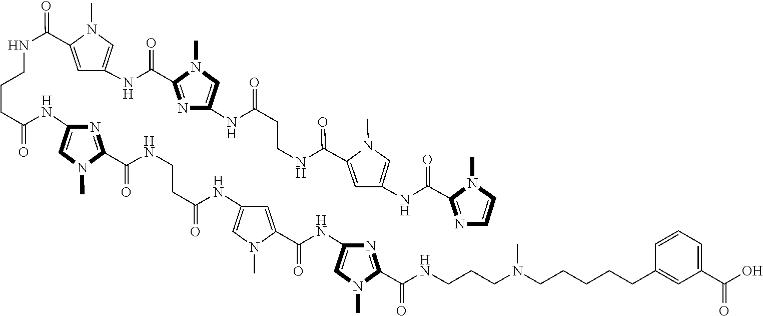 | B-215 |
| 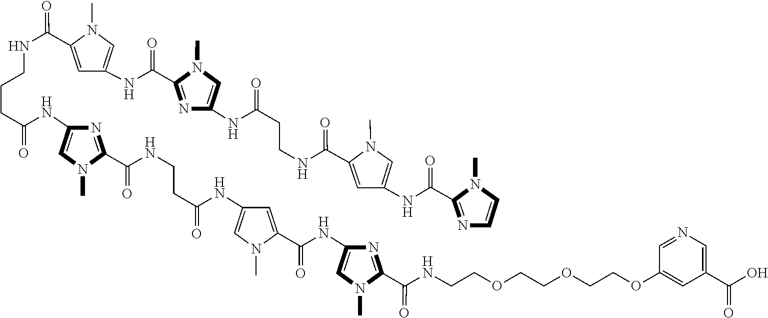 | B-216 |
| 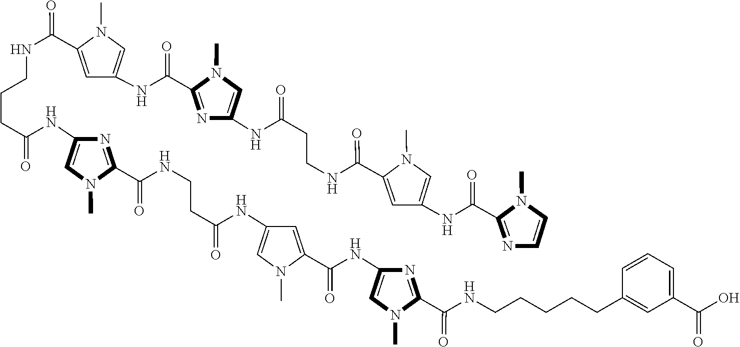 | B-217 |
| 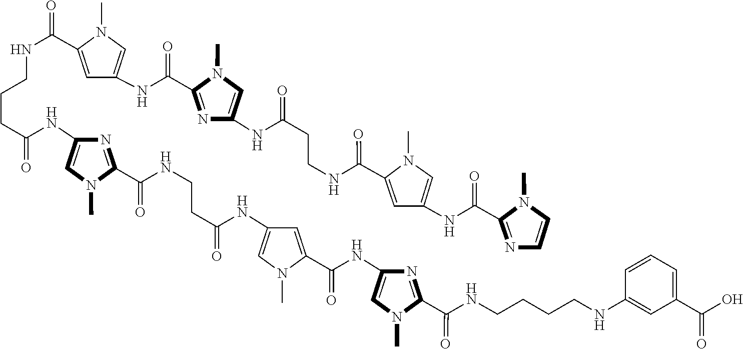 | B-218 |
| 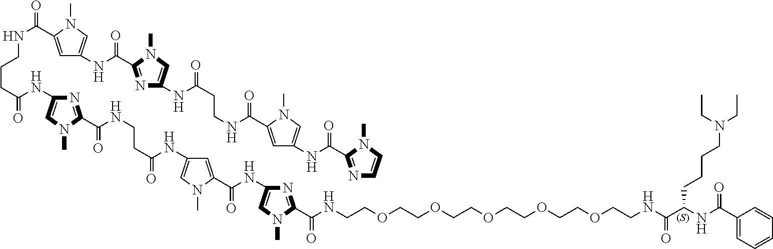 | B-219 |
| 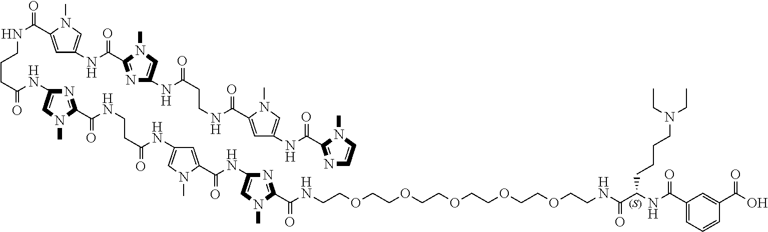 | B-220 |
| 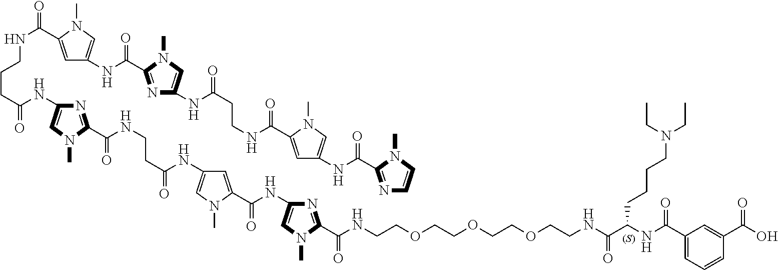 | B-221 |
| 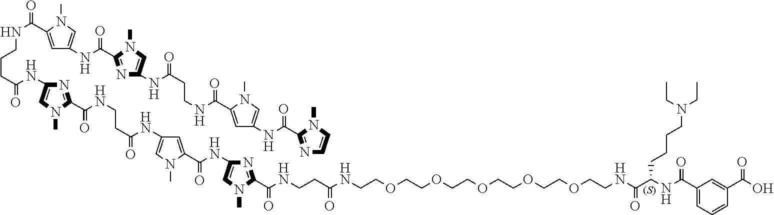 | B-222 |
| 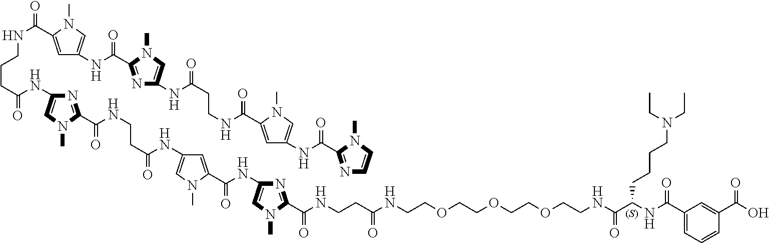 | B-223 |
| 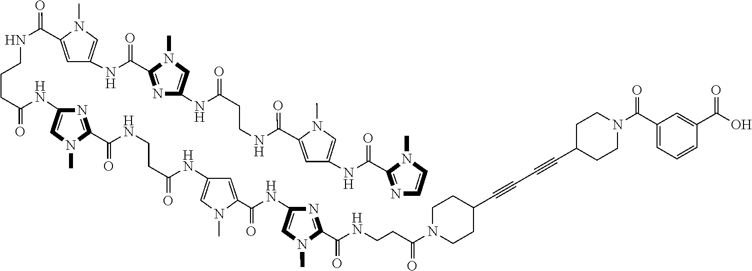 | B-224 |
| 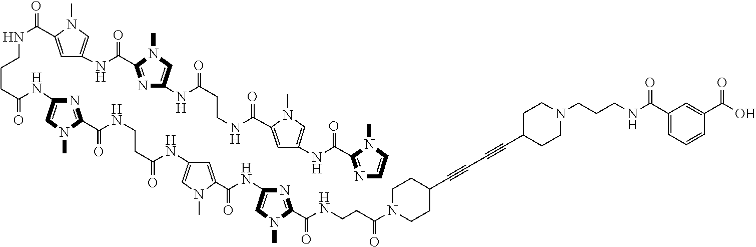 | B-225 |
| 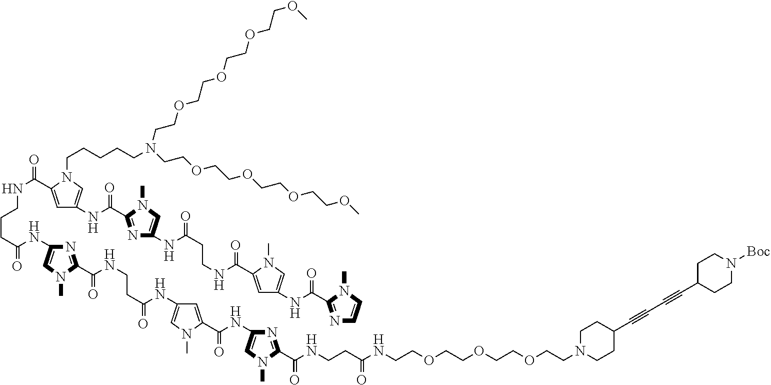 | B-226 |
| 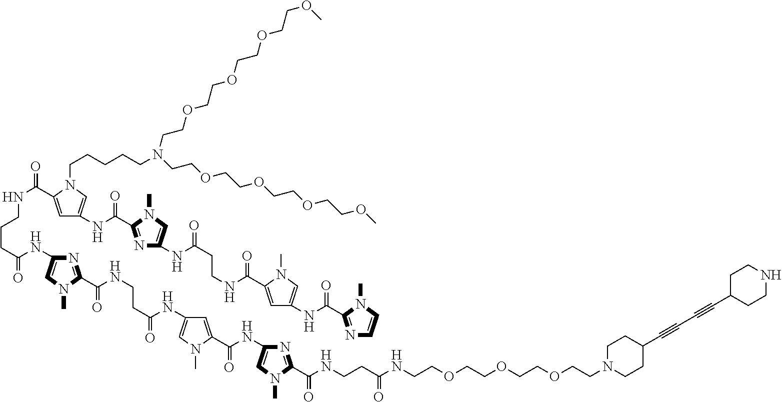 | B-227 |
| 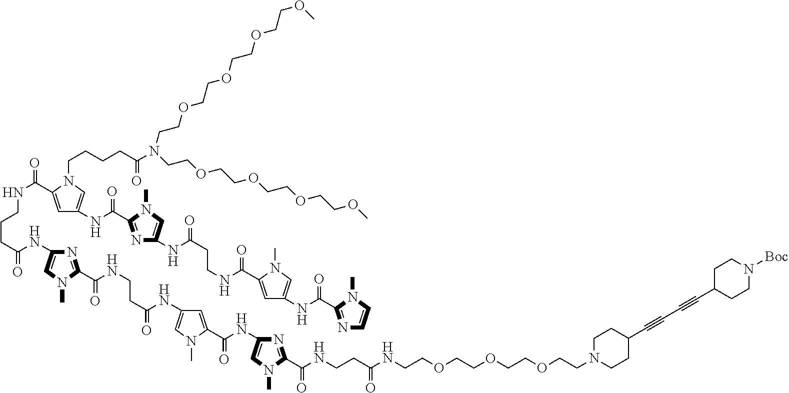 | B-228 |
| 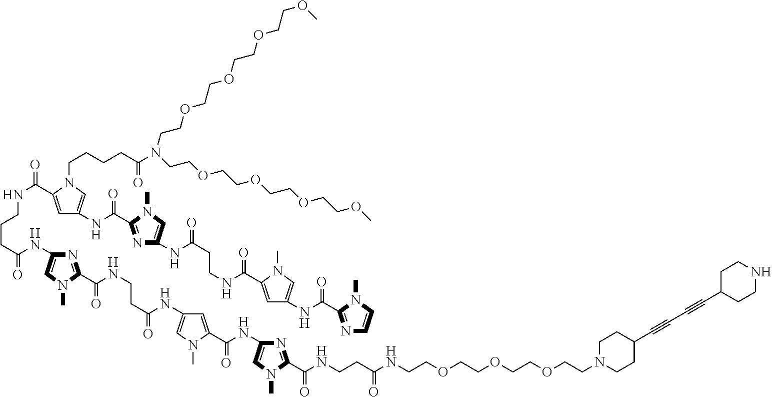 | B-229 |
| 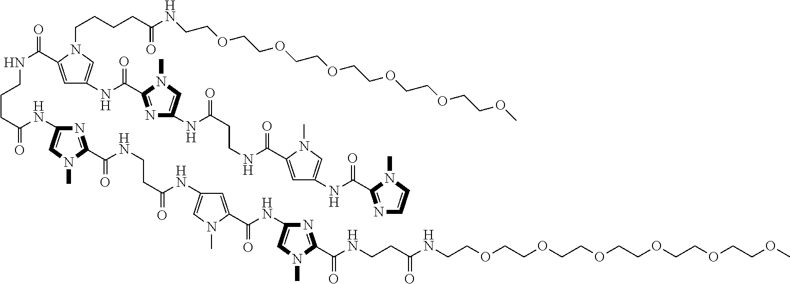 | B-230 |
| 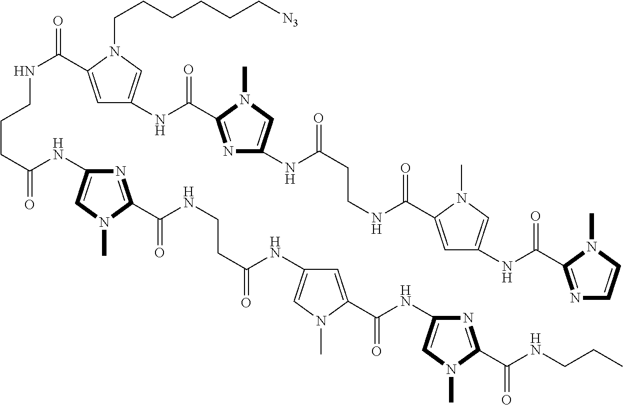 | B-231 |
| 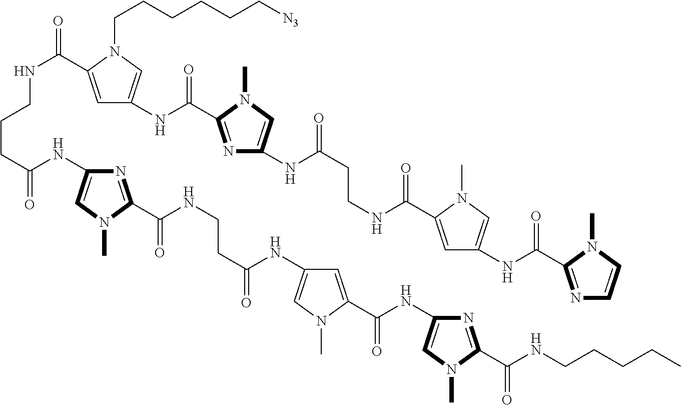 | B-232 |
| 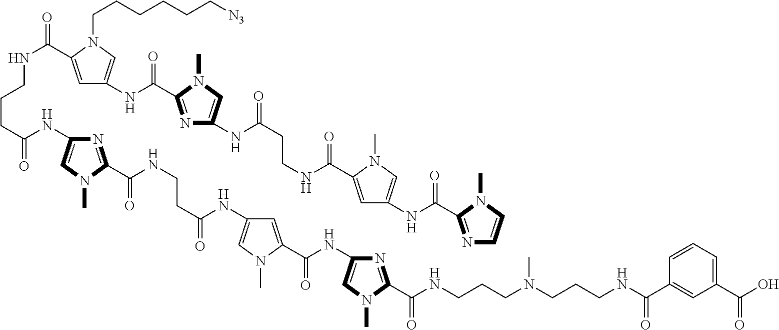 | B-233 |
| 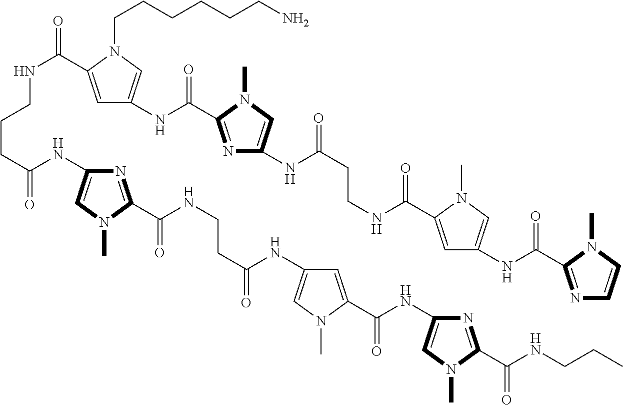 | B-234 |
| 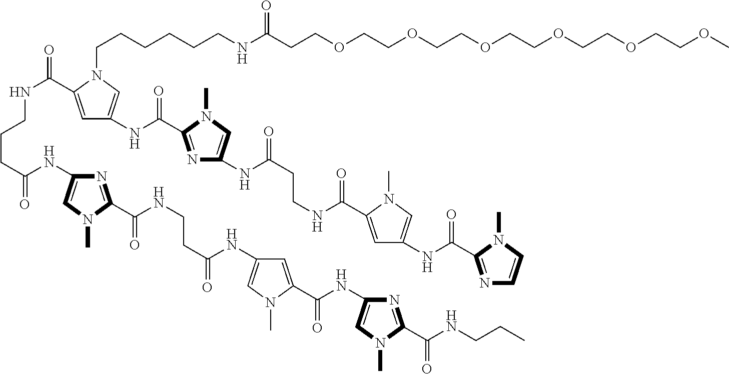 | B-235 |
| 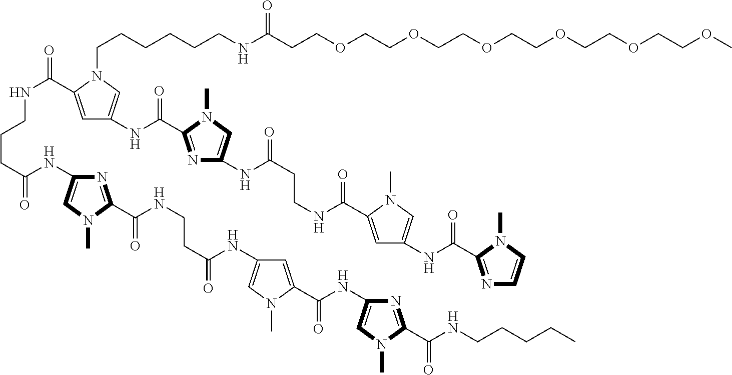 | B-236 |
| 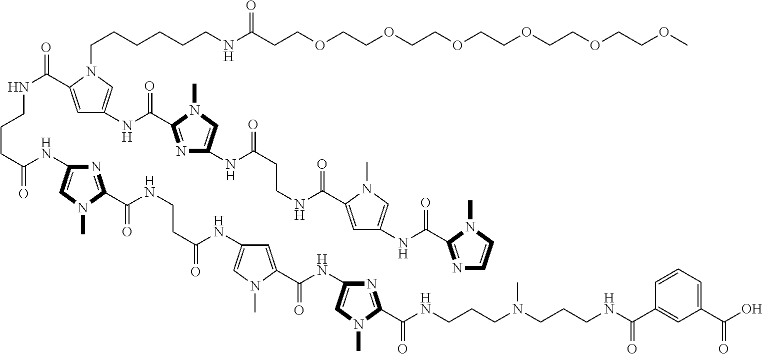 | B-237 |
| 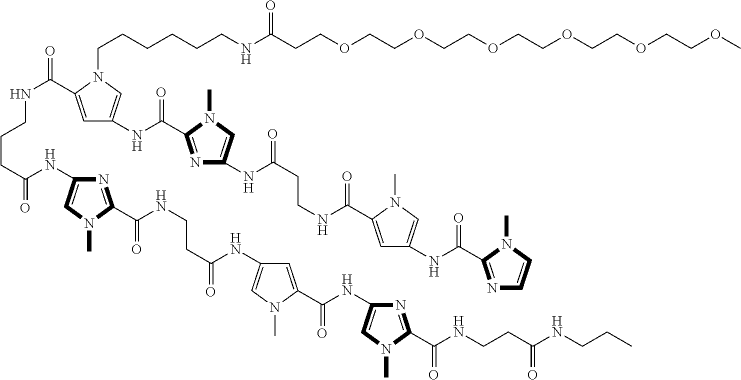 | B-238 |
| 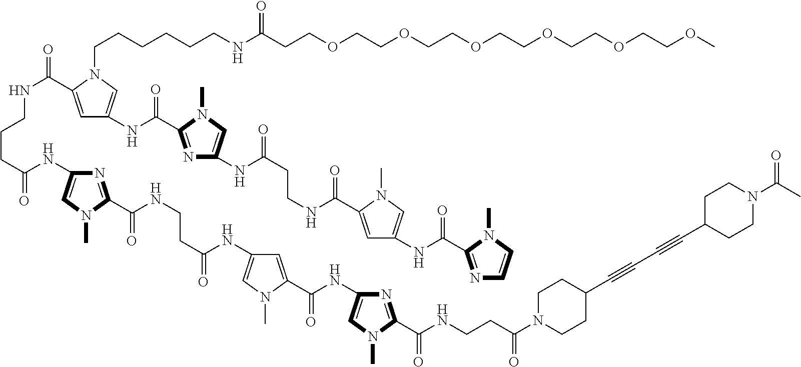 | B-239 |
| 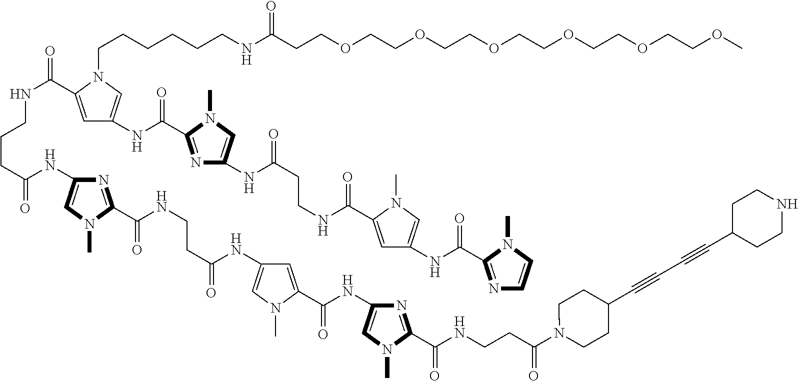 | B-240 |
| 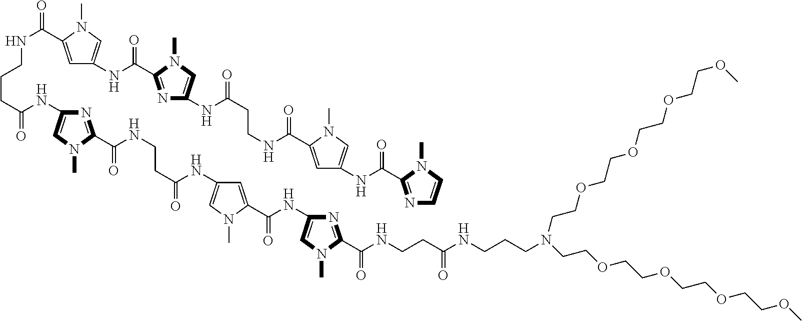 | B-241 |
| 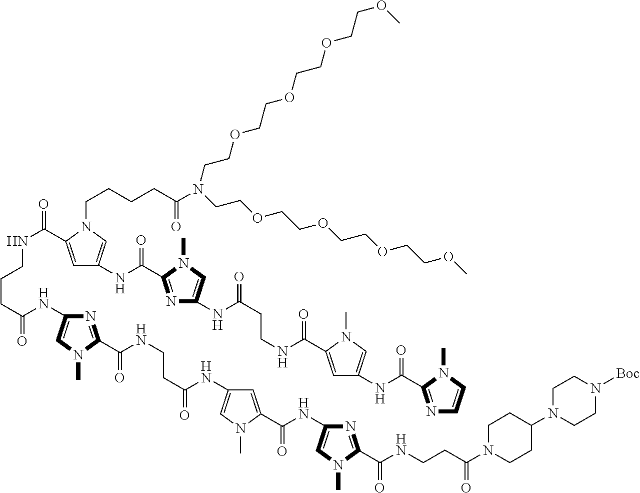 | B-242 |
| 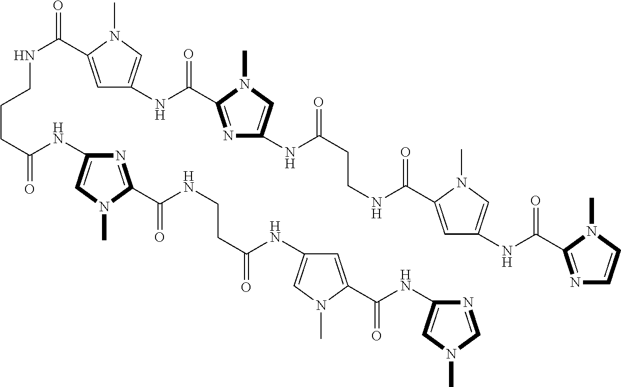 | B-243 |
| 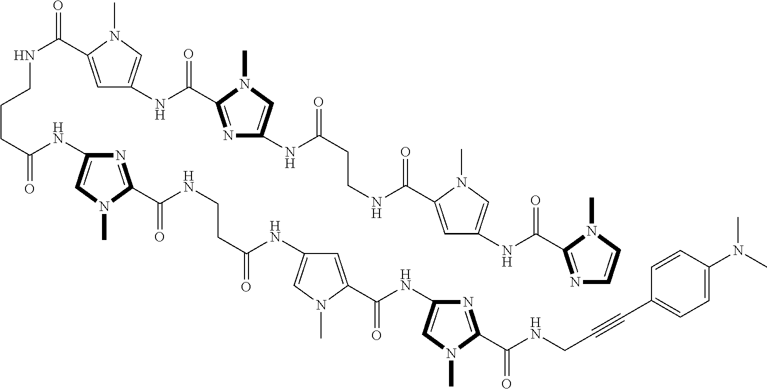 | B-244 |
| 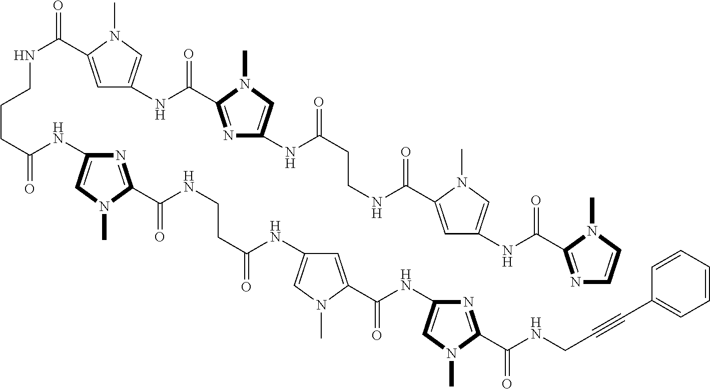 | B-245 |
| 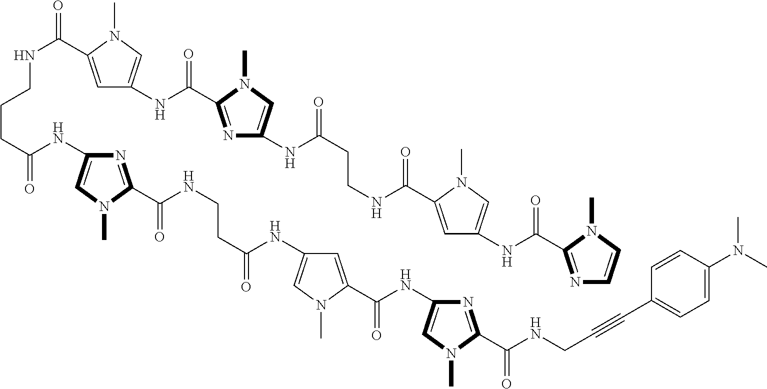 | B-246 |
| 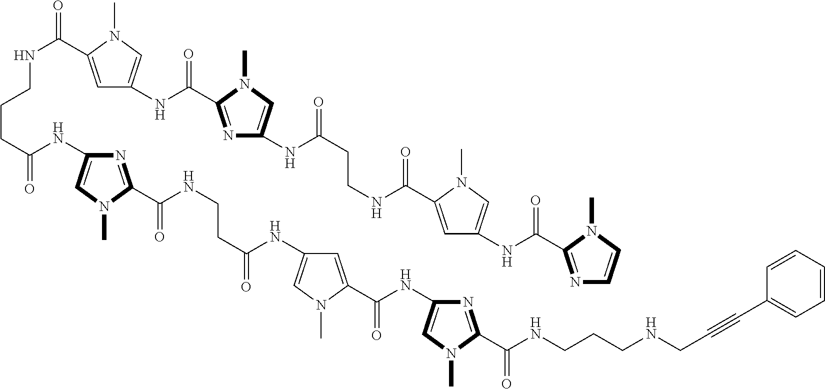 | B-247 |
| 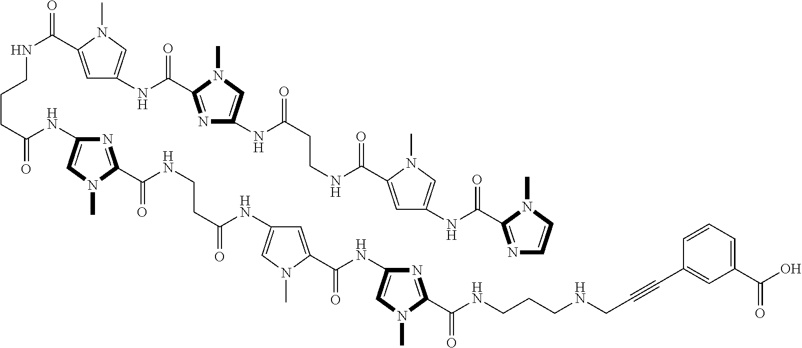 | B-248 |
| 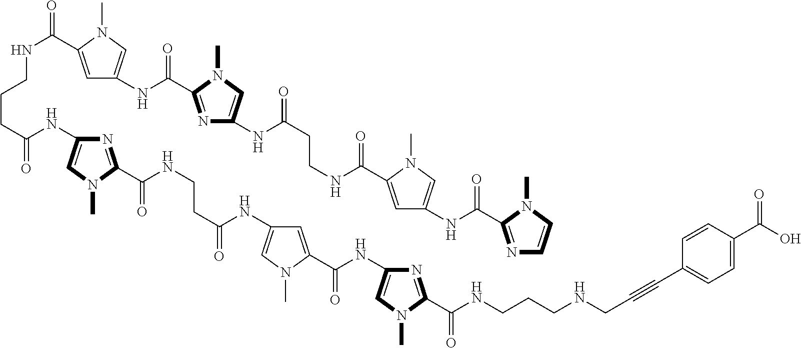 | B-249 |
| 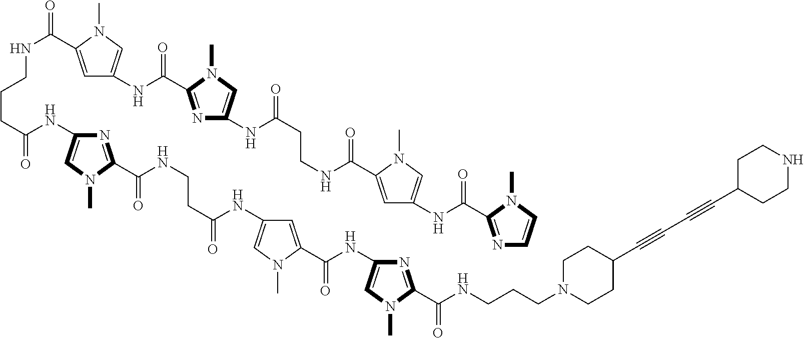 | B-250 |
| 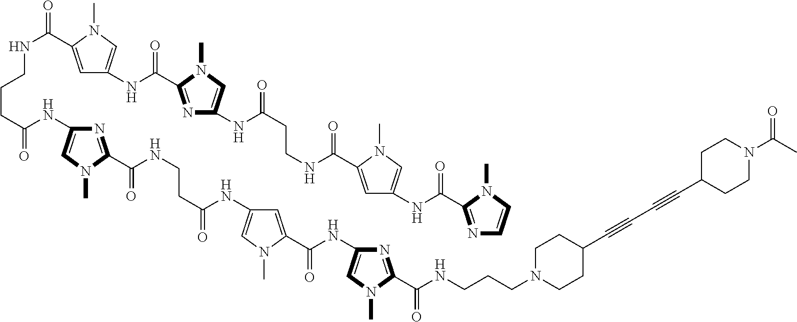 | B-251 |
| 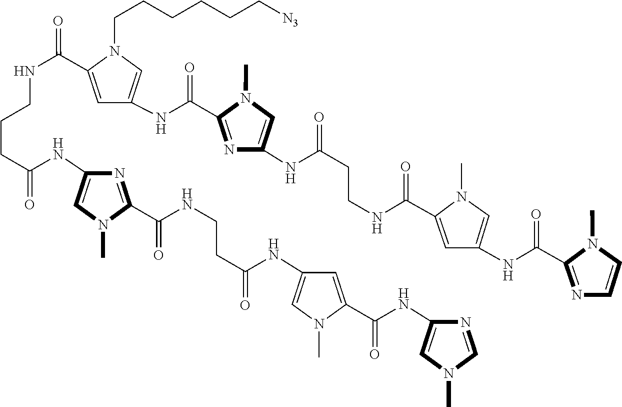 | B-252 |
| 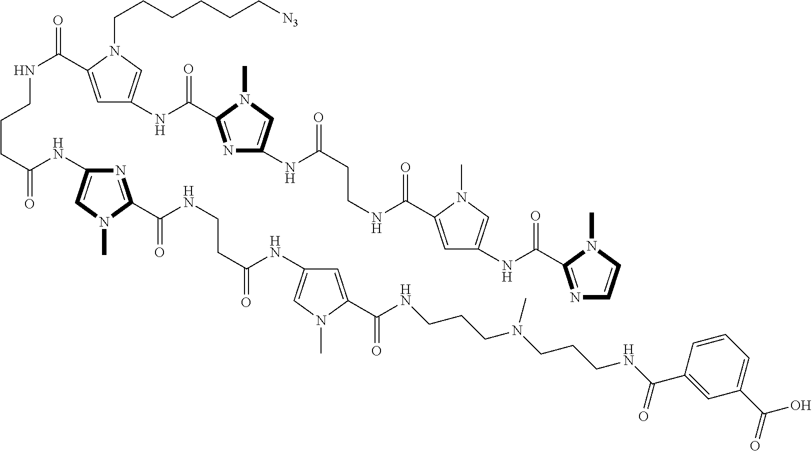 | B-253 |
| 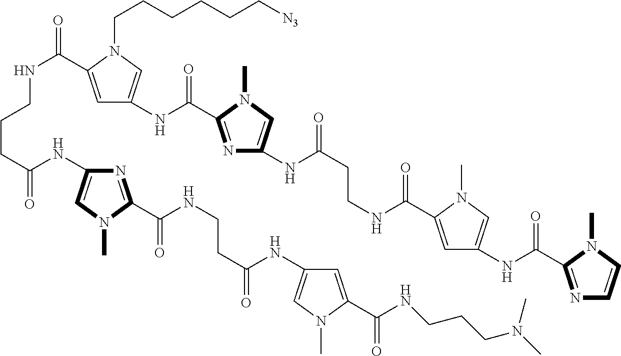 | B-254 |
| 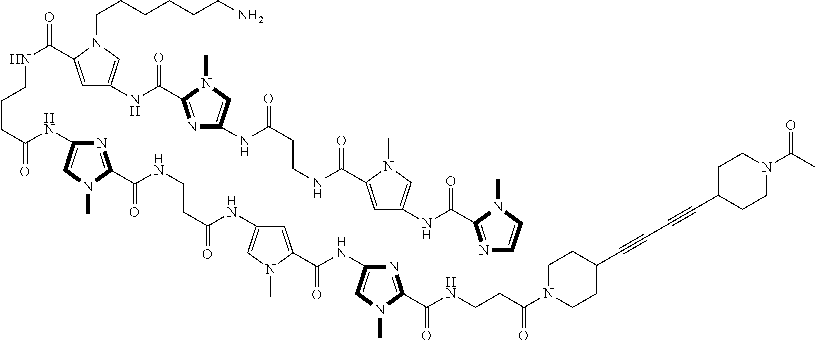 | B-255 |
| 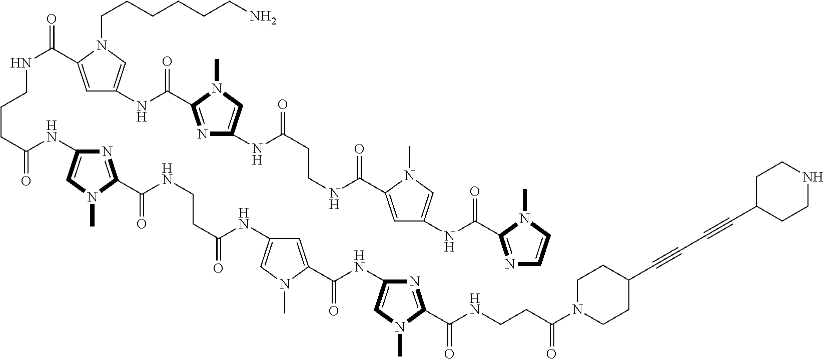 | B-256 |
| 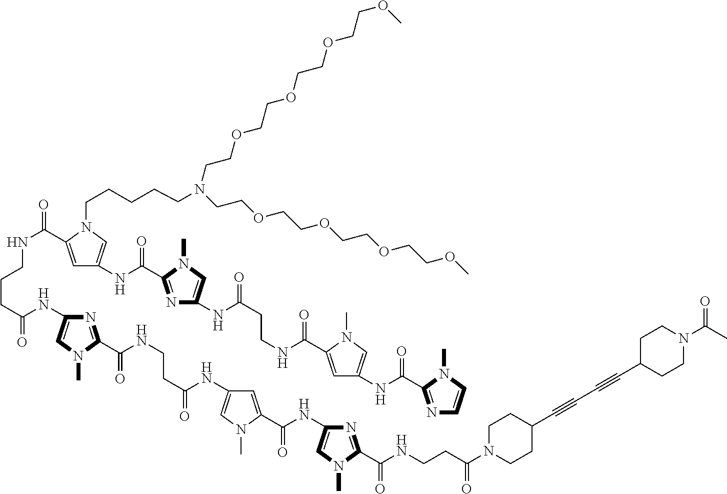 | B-257 |
| 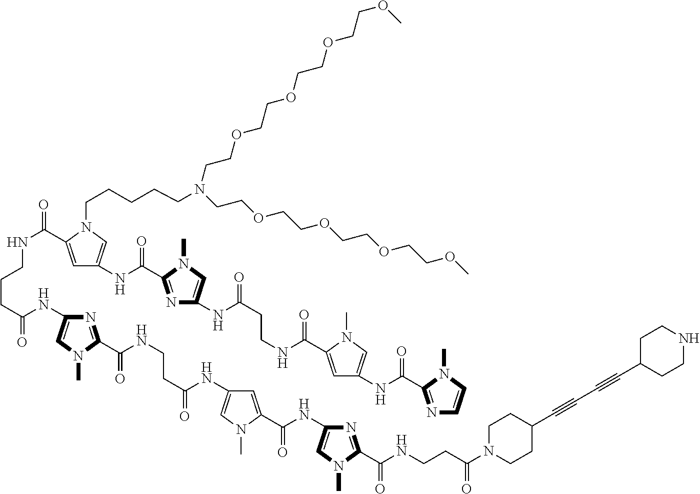 | B-258 |
| 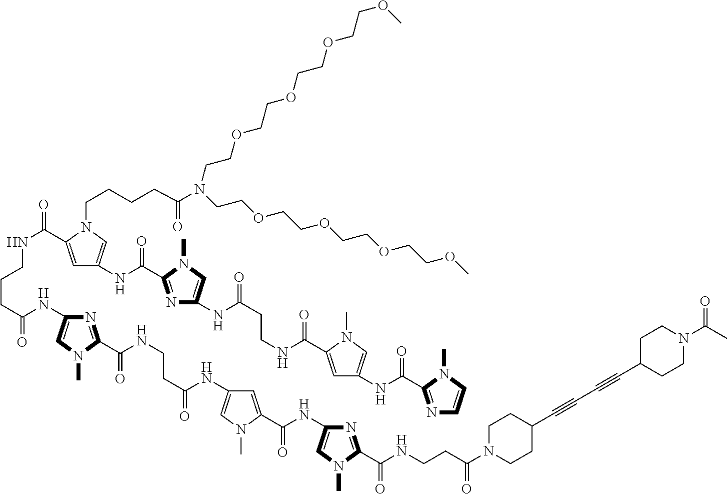 | B-259 |
| 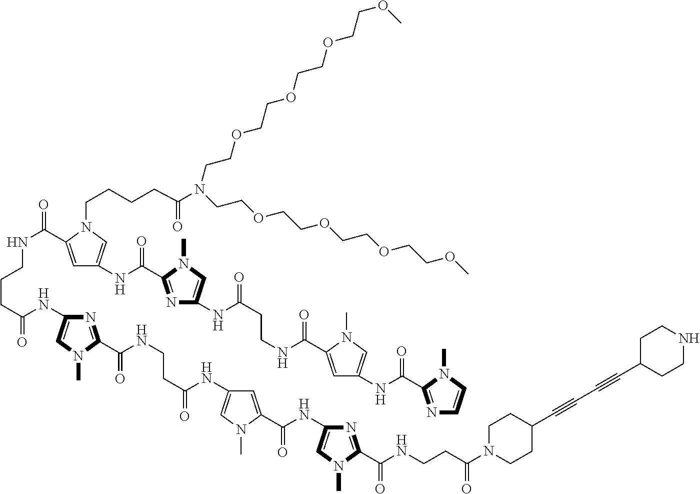 | B-260 |
| 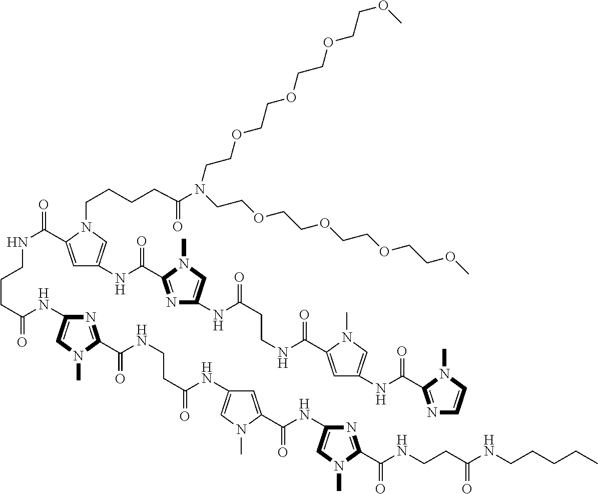 | B-261 |
| 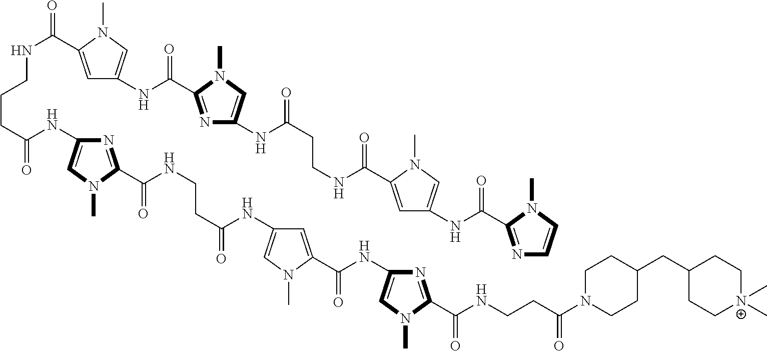 | B-262 |
| 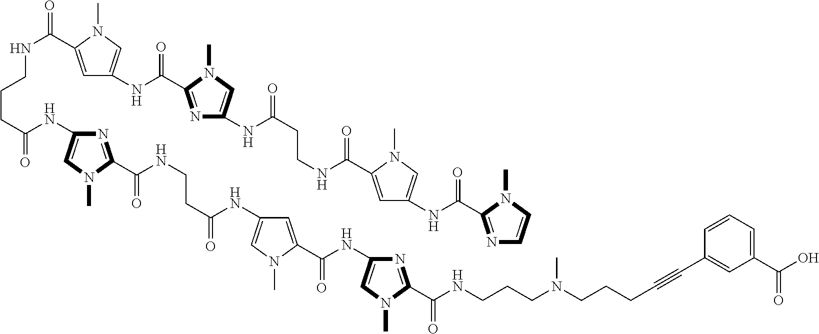 | B-263 |
| 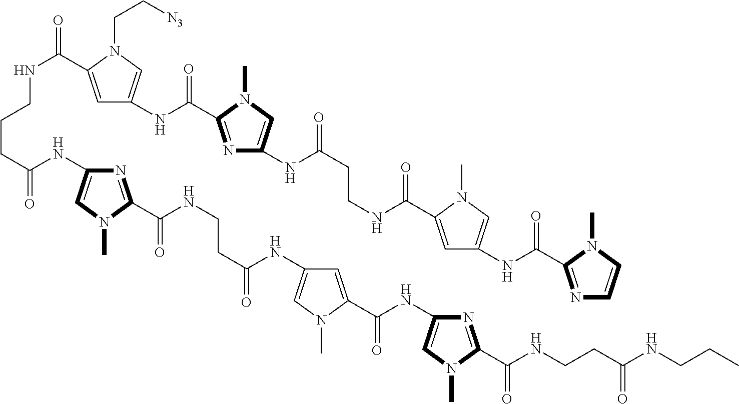 | B-264 |
| 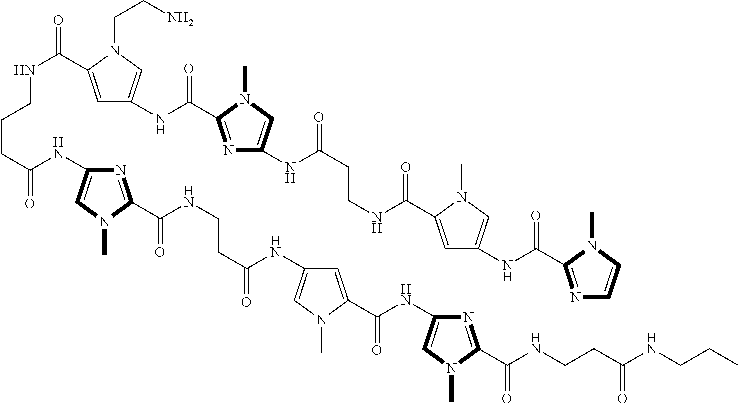 | B-265 |
| 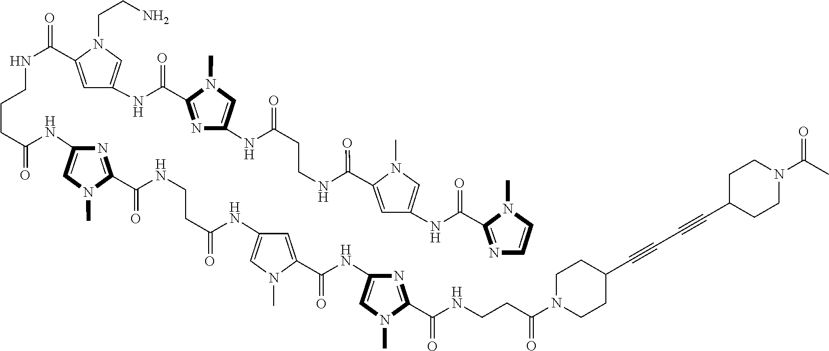 | B-266 |
| 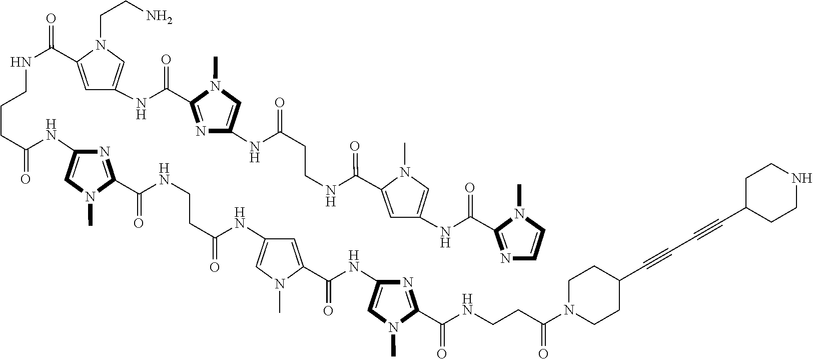 | B-267 |
| 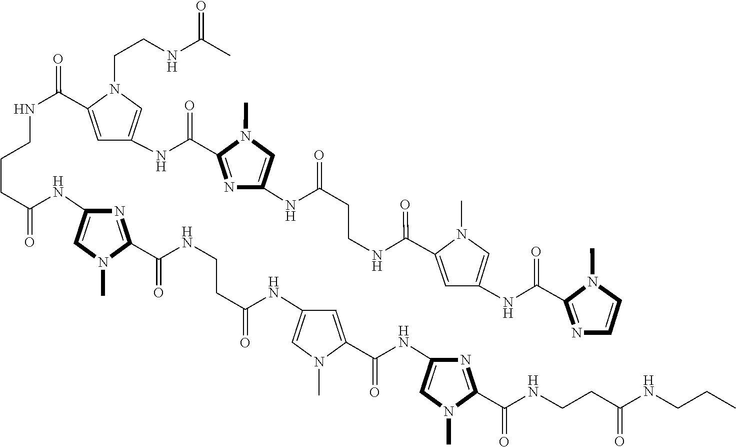 | B-268 |
| 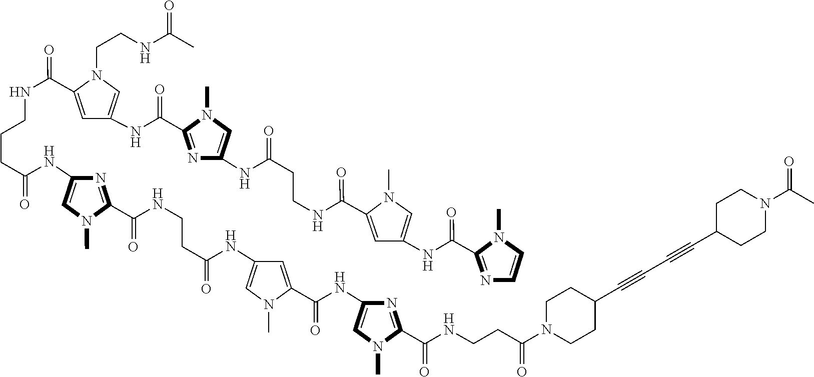 | B-269 |
| 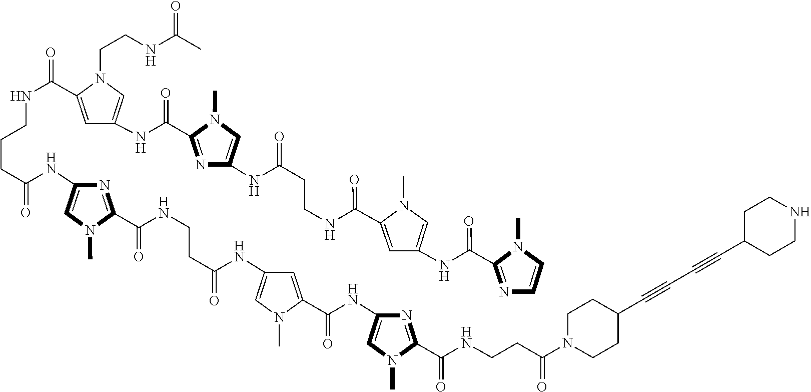 | B-270 |
| 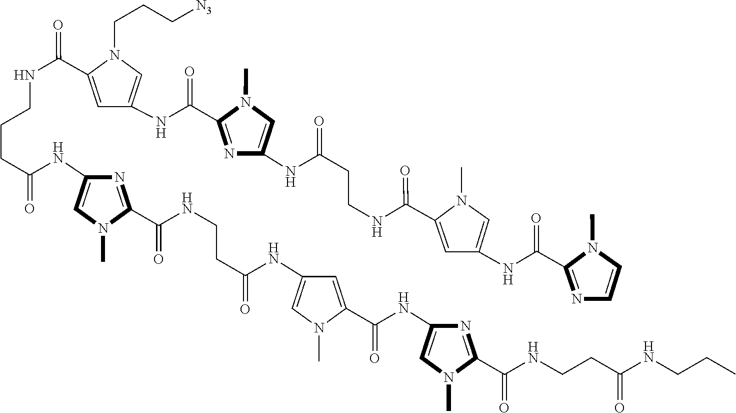 | B-271 |
| 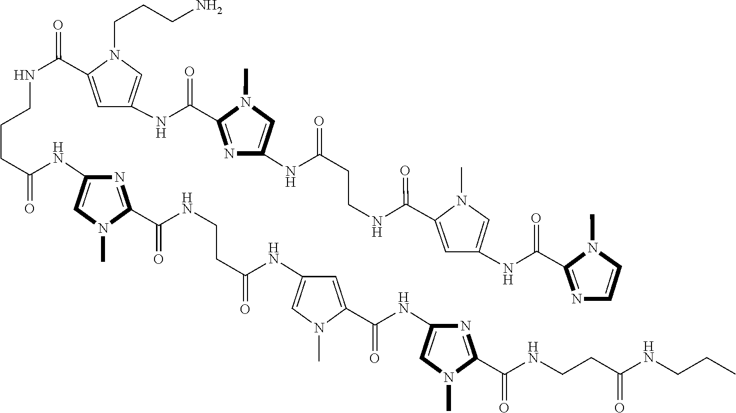 | B-272 |
| 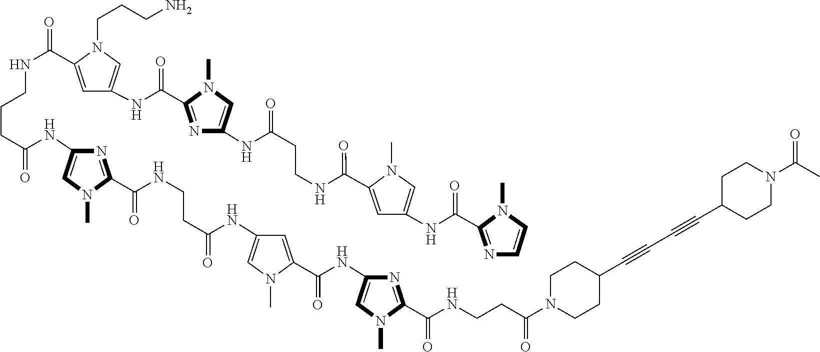 | B-273 |
| 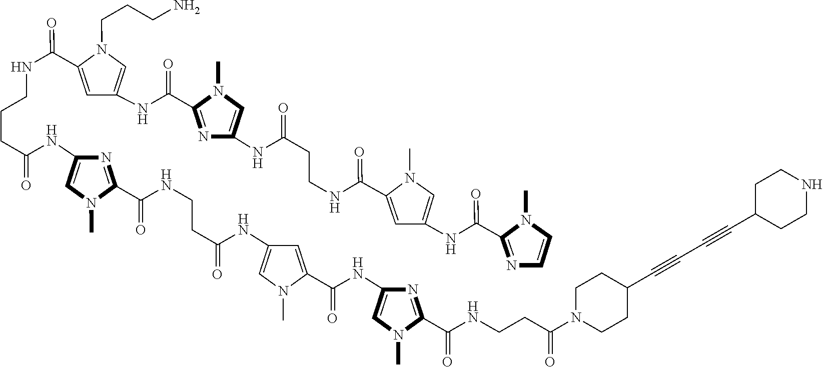 | B-274 |
| 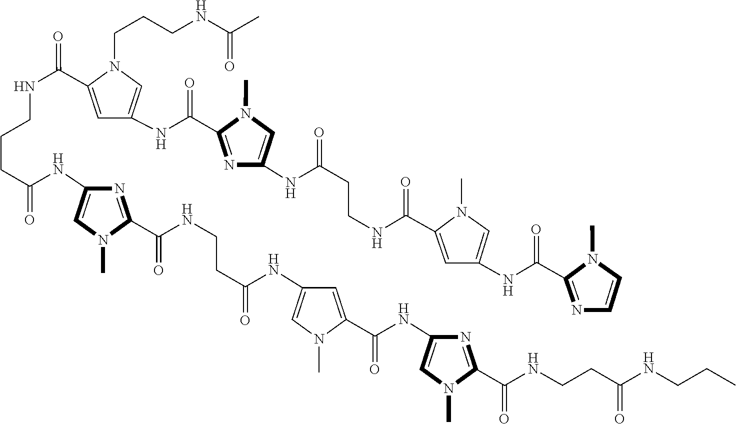 | B-275 |
| 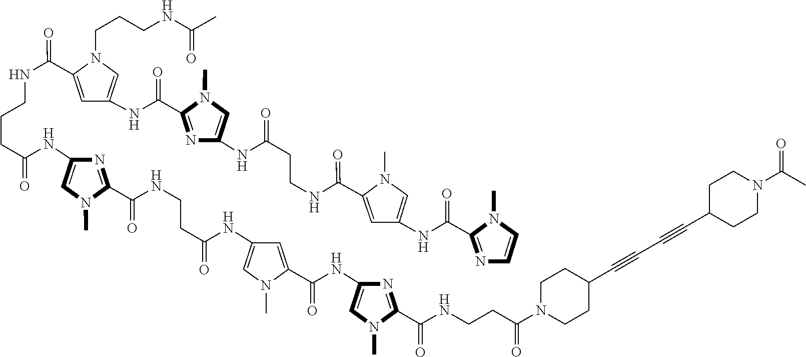 | B-276 |
| 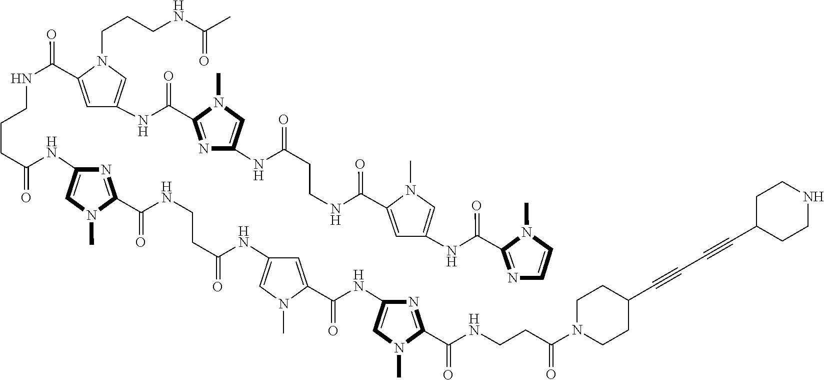 | B-277 |
| 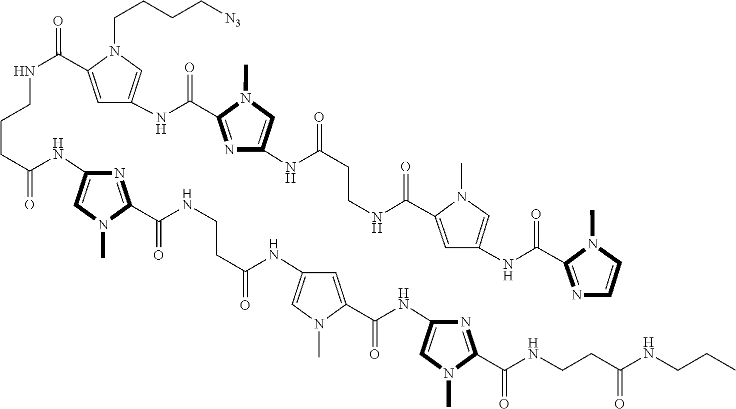 | B-278 |
| 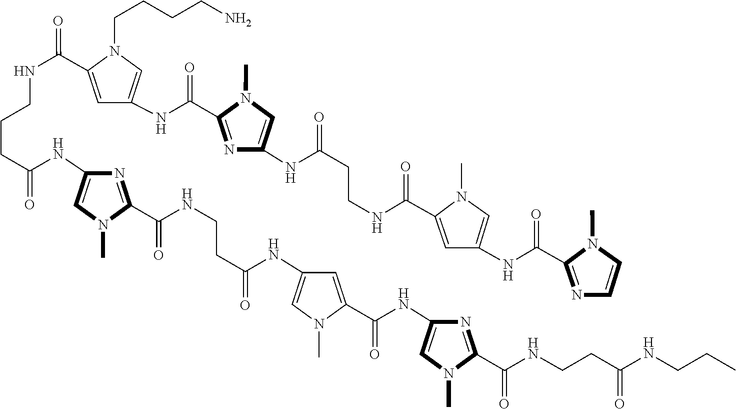 | B-279 |
| 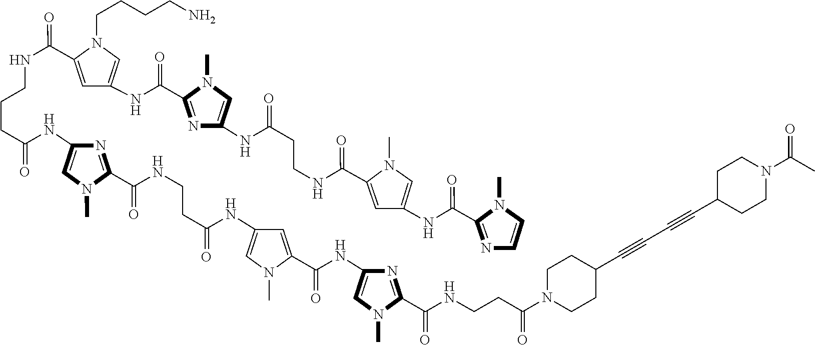 | B-280 |
| 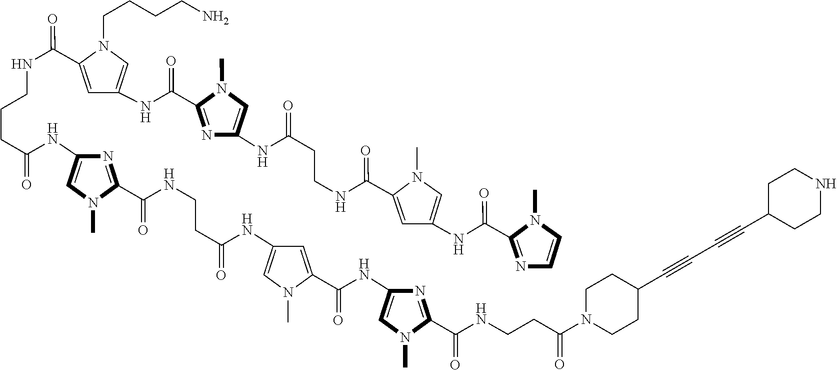 | B-281 |
| 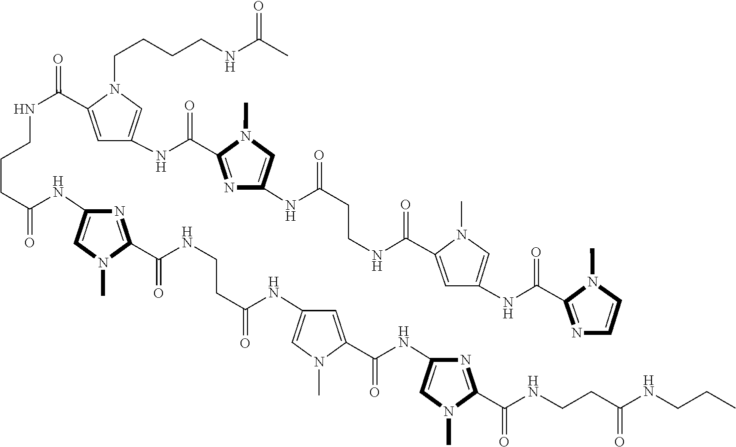 | B-282 |
| 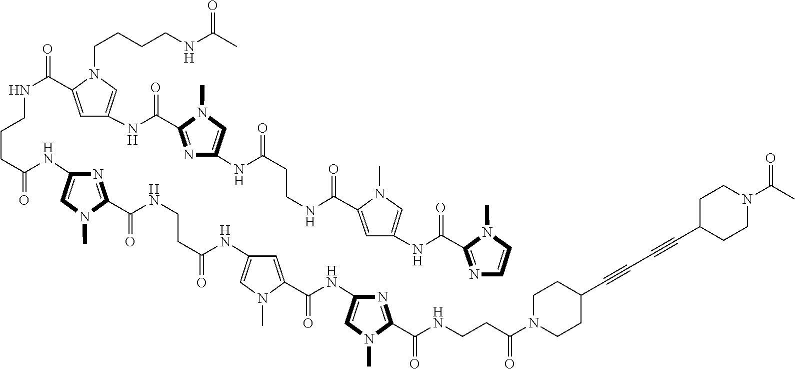 | B-283 |
| 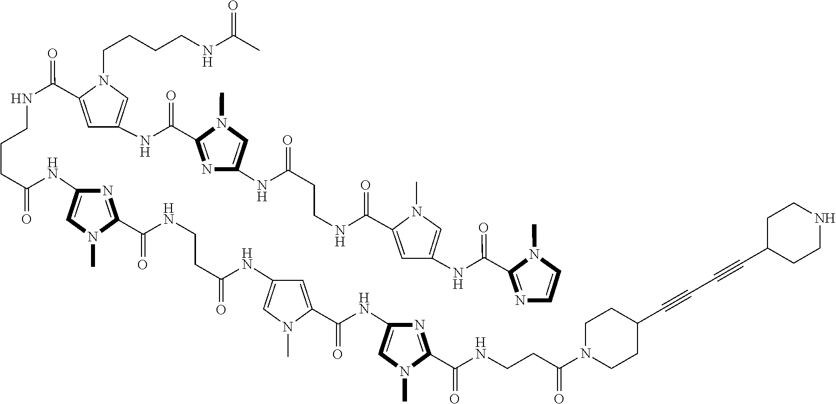 | B-284 |
| 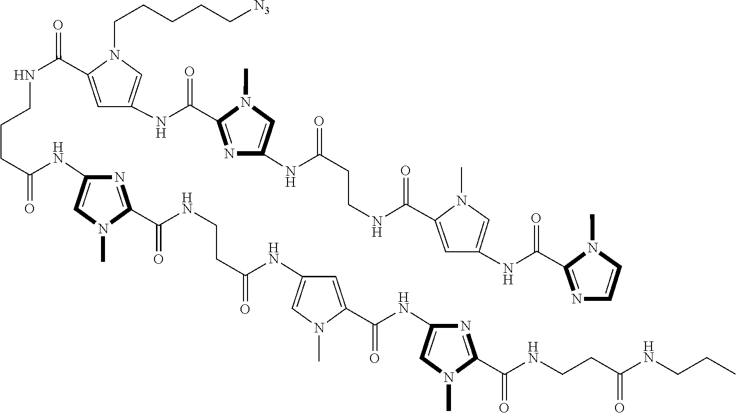 | B-285 |
| 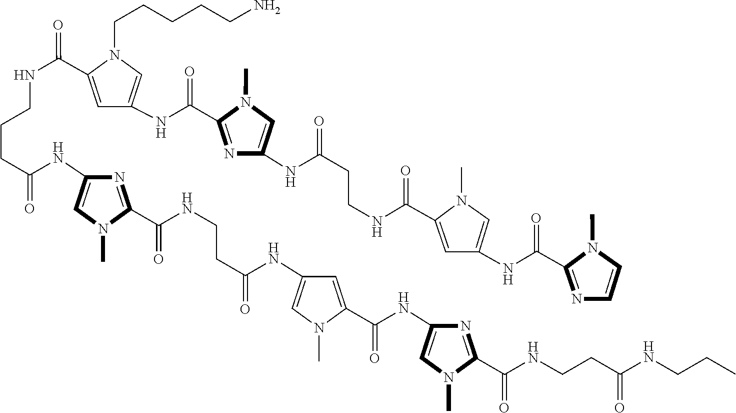 | B-286 |
| 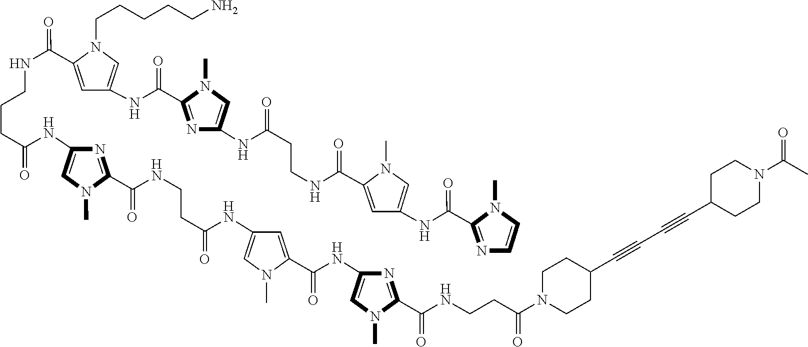 | B-287 |
| 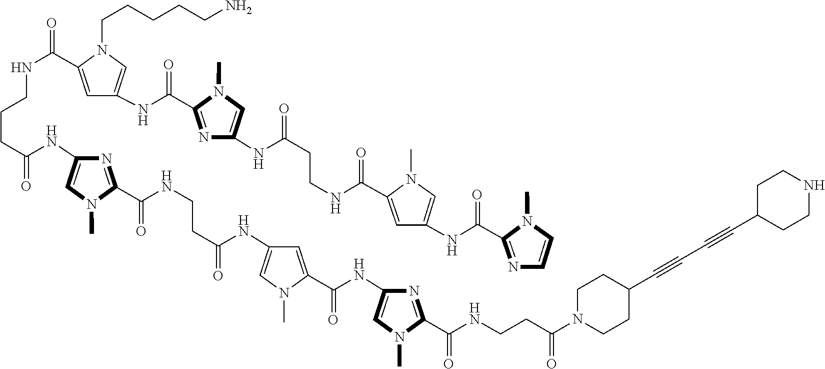 | B-288 |
| 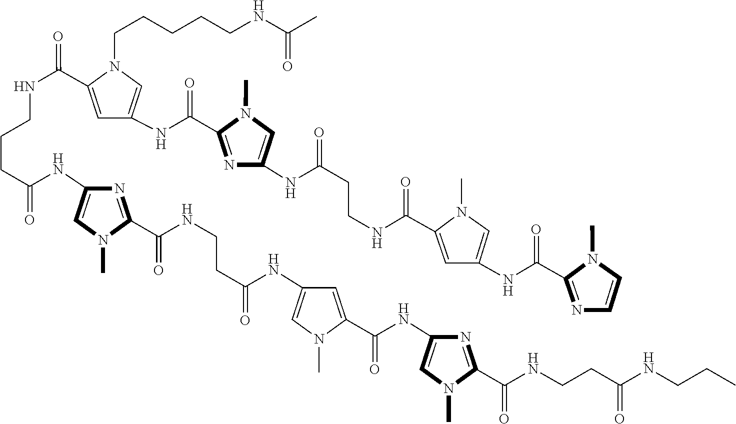 | B-289 |
| 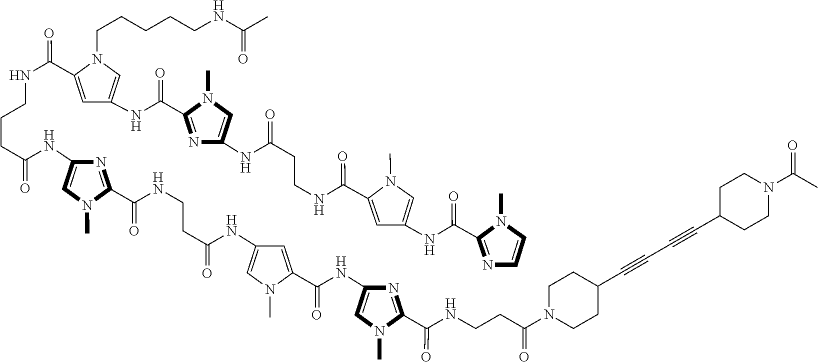 | B-290 |
| 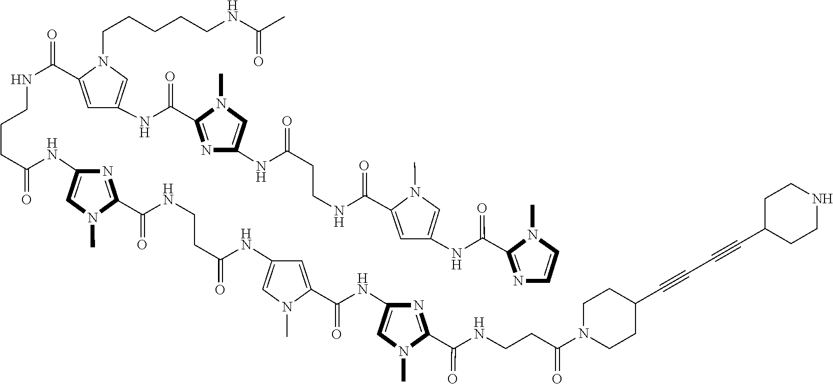 | B-291 |
| 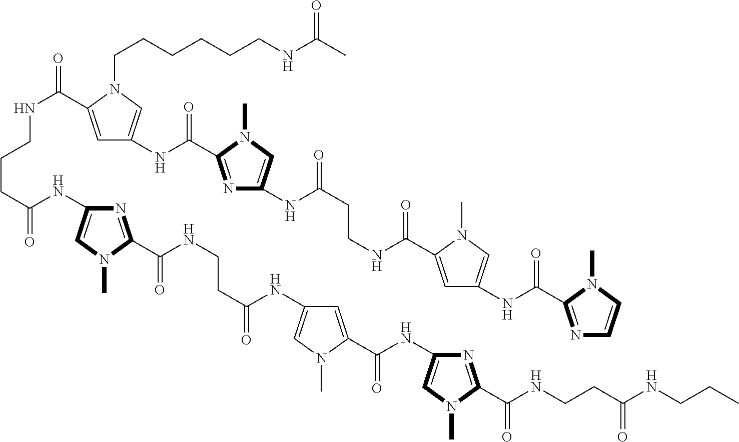 | B-292 |
| 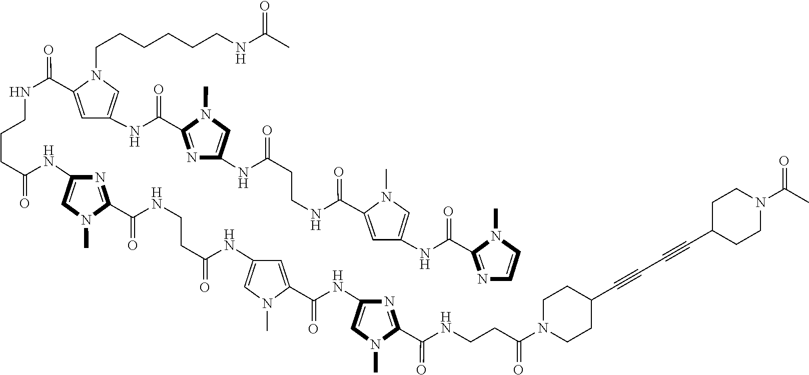 | B-293 |
| 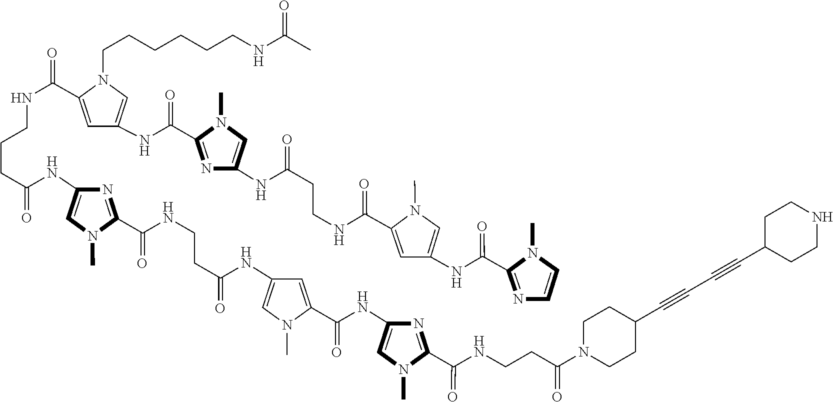 | B-294 |
| 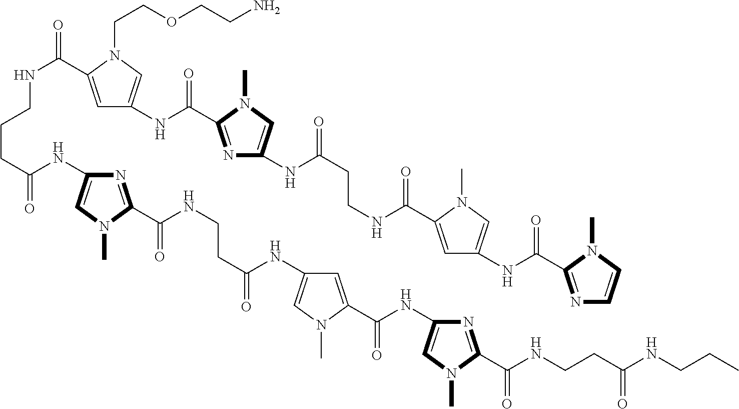 | B-295 |
| 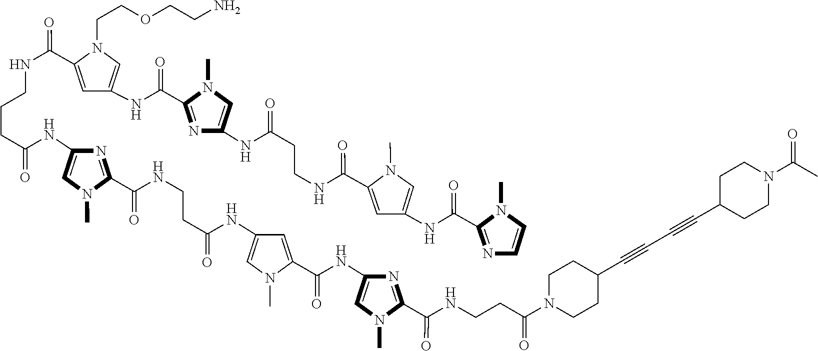 | B-296 |
| 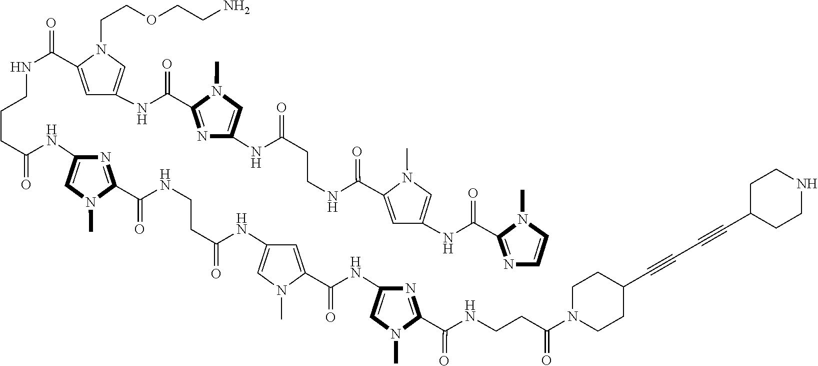 | B-297 |
| 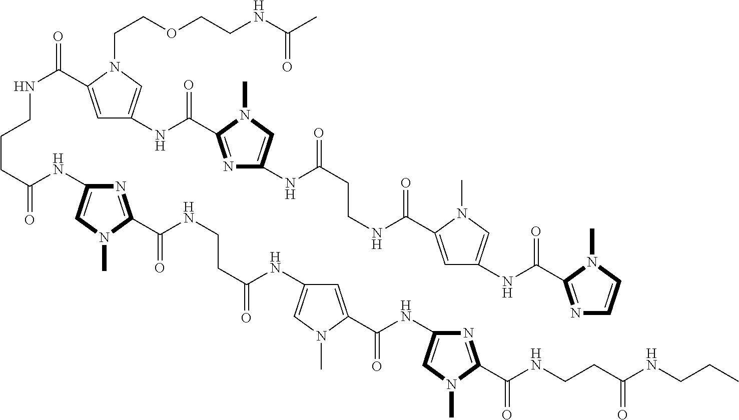 | B-298 |
| 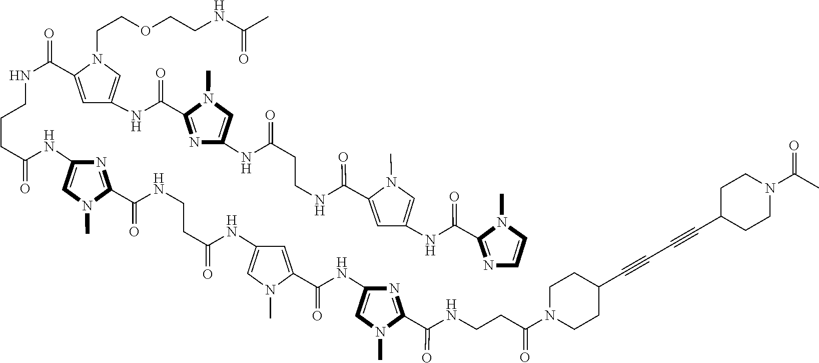 | B-299 |
| 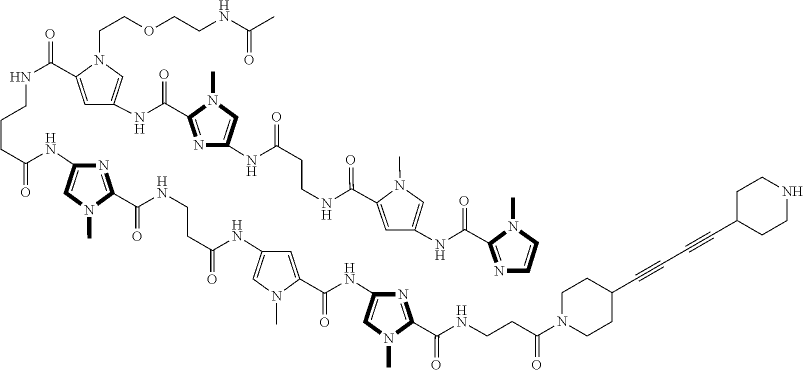 | B-300 |
| 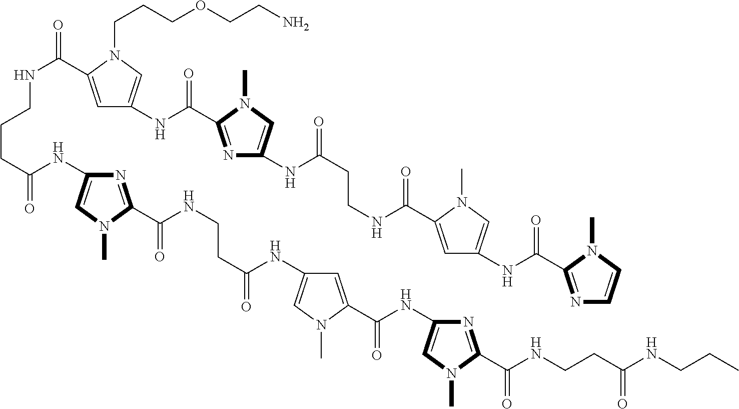 | B-301 |
| 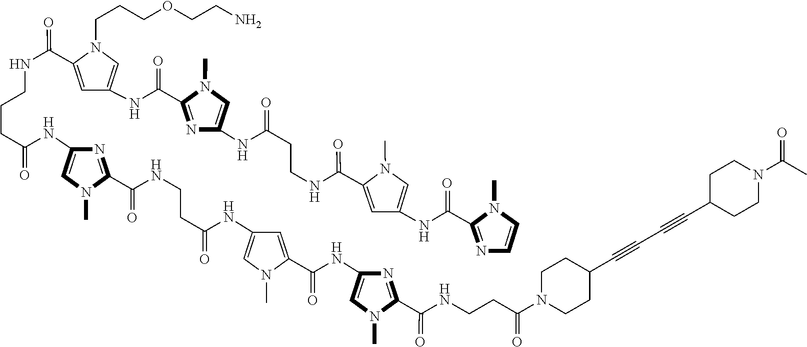 | B-302 |
| 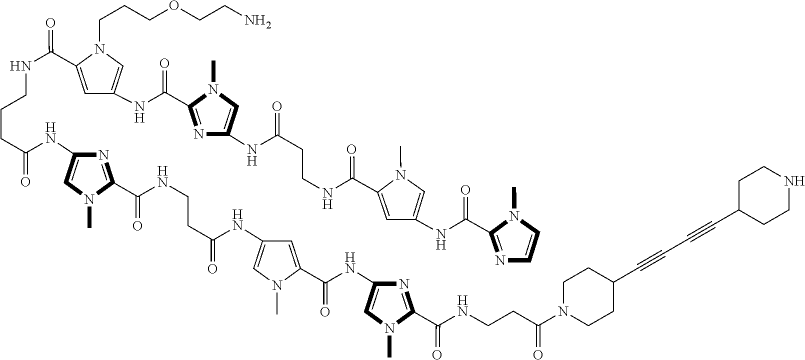 | B-303 |
| 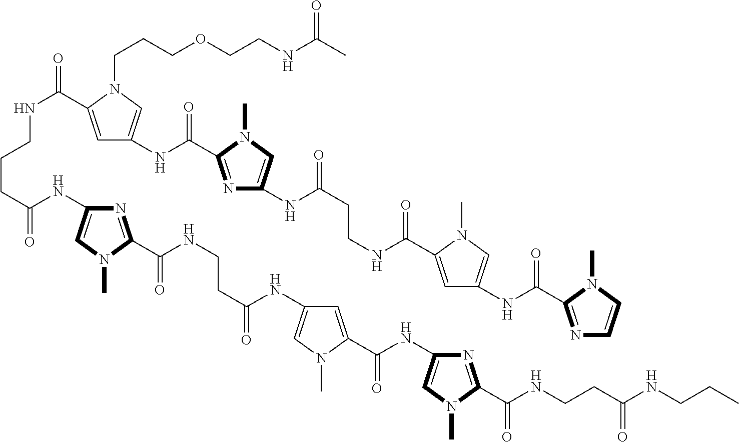 | B-304 |
| 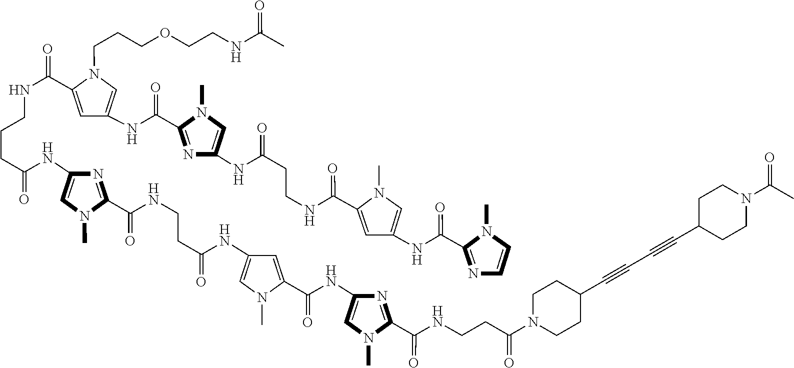 | B-305 |
| 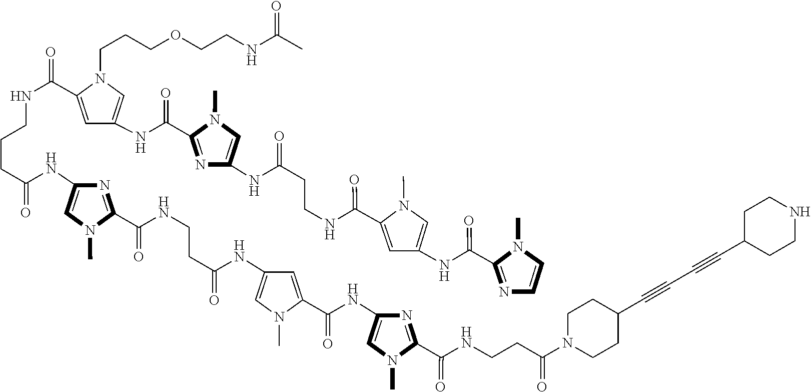 | B-306 |
| 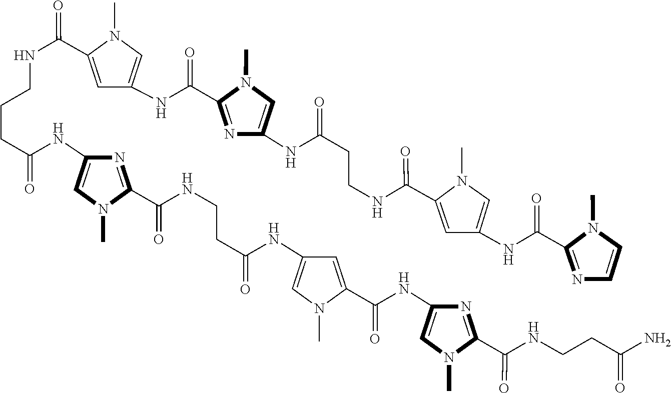 | B-307 |
| 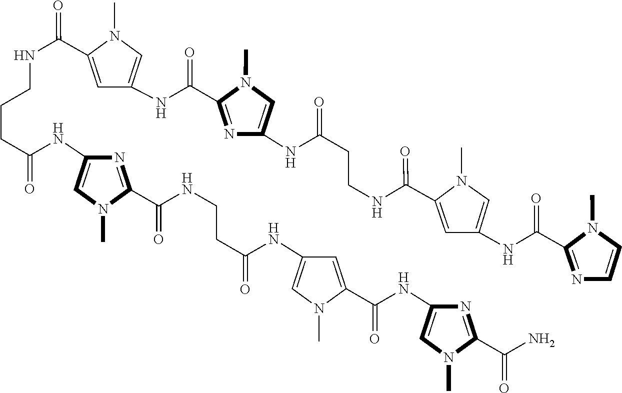 | B-308 |
| 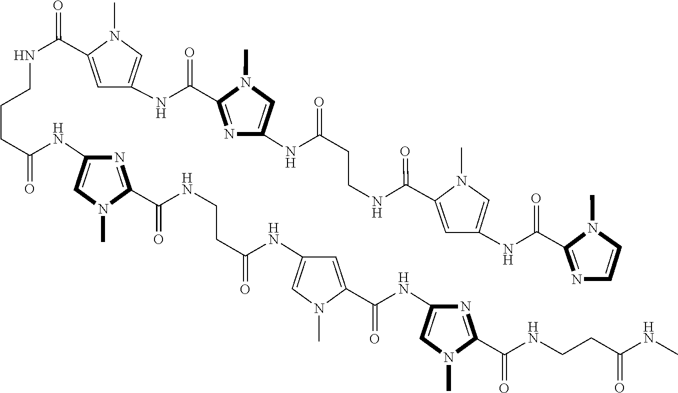 | B-309 |
| 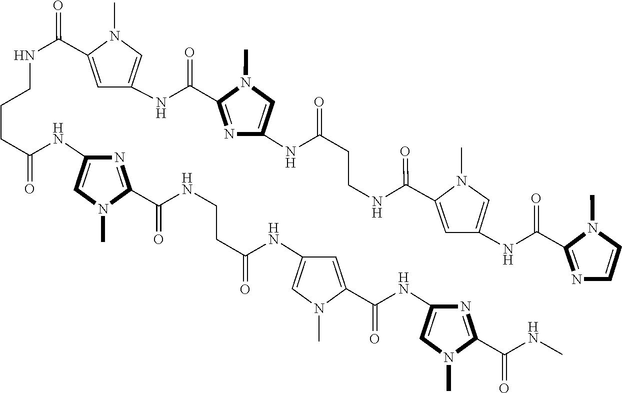 | B-310 |
| 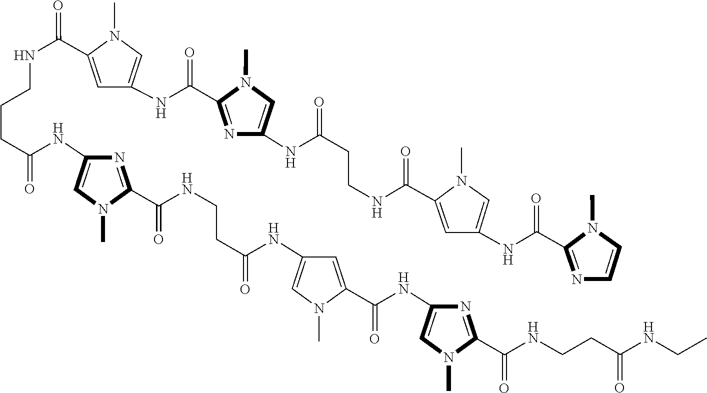 | B-311 |
| 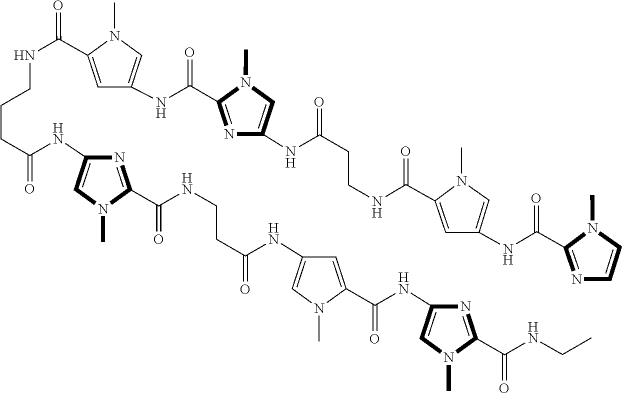 | B-312 |
| 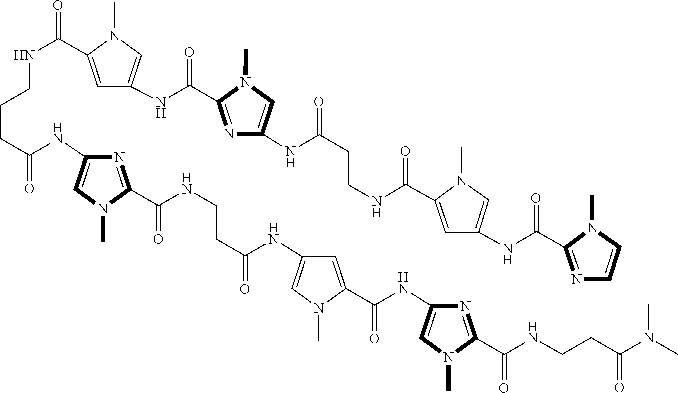 | B-313 |
| 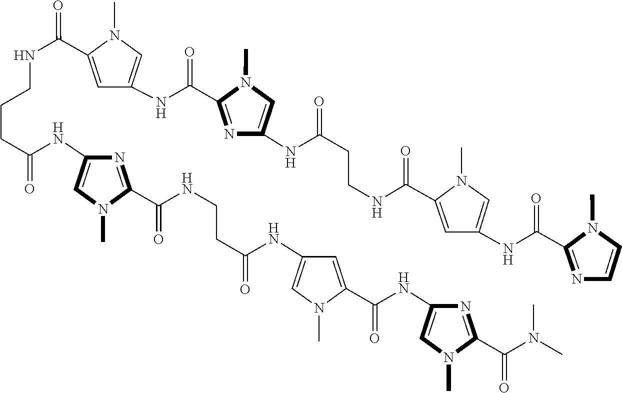 | B-314 |
| 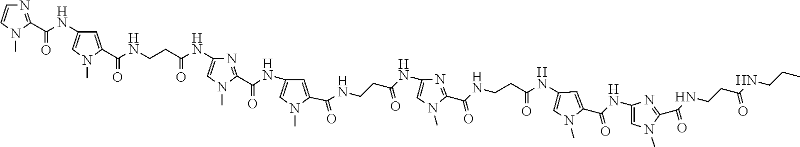 [SMILES: CCCNC(=O)CCNC(=O)c1nc(NC(=O)c2cc(NC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)cn2C)cn1C] | B-315 |
| 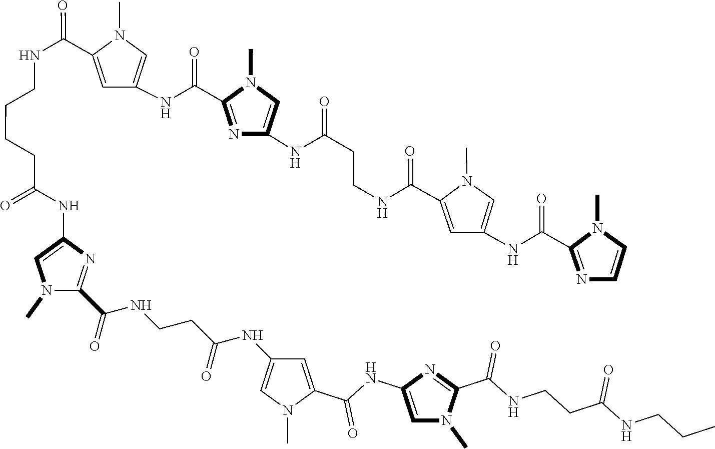 | B-317 |
| 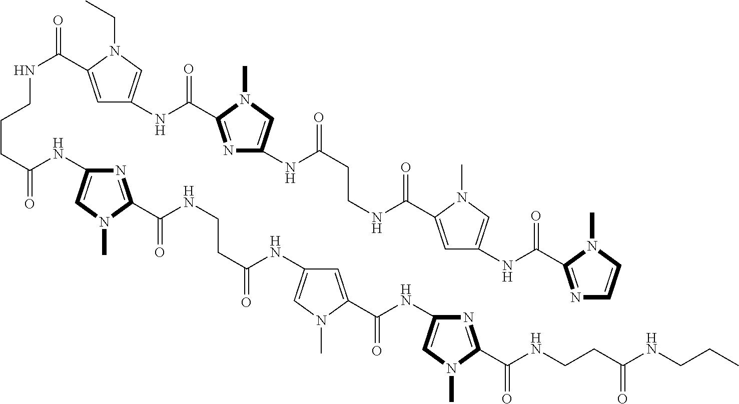 | B-318 |
| 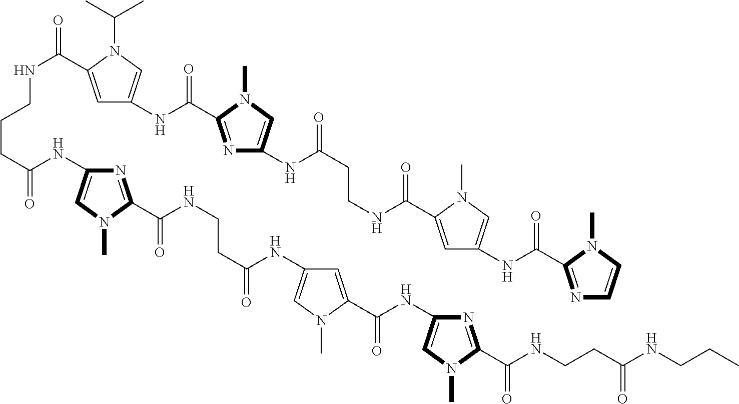 | B-319 |
| 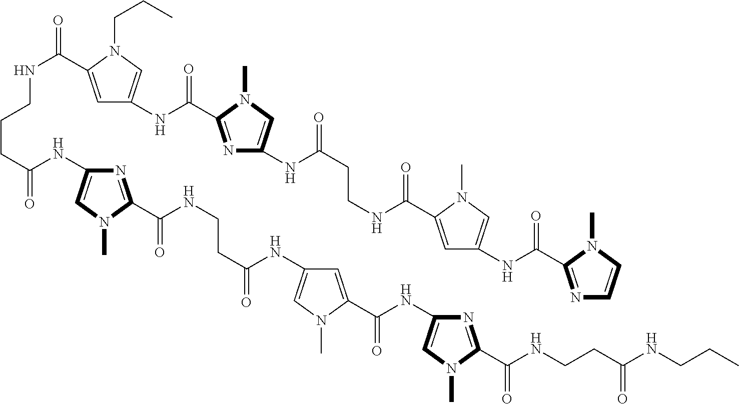 | B-320 |
| 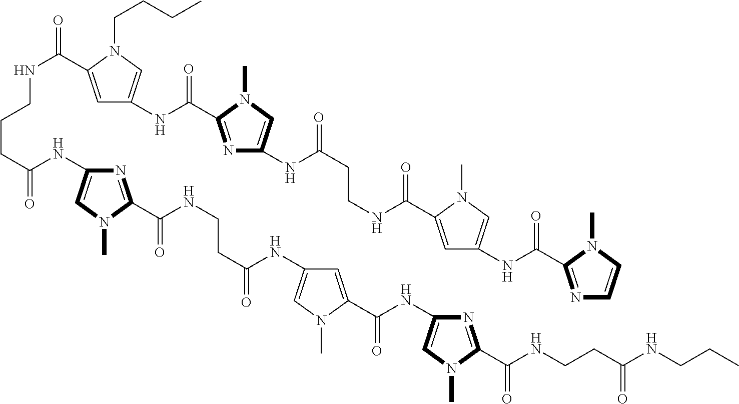 | B-321 |
| 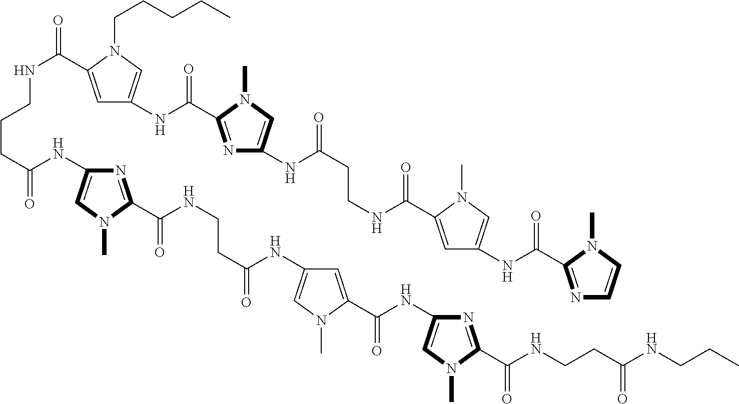 | B-322 |
| 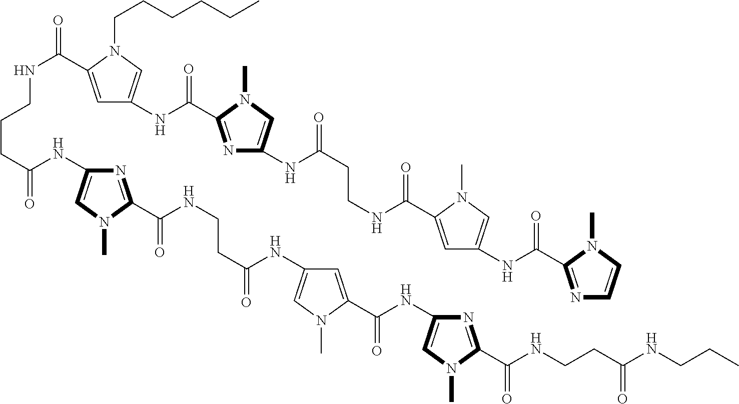 | B-323 |
| 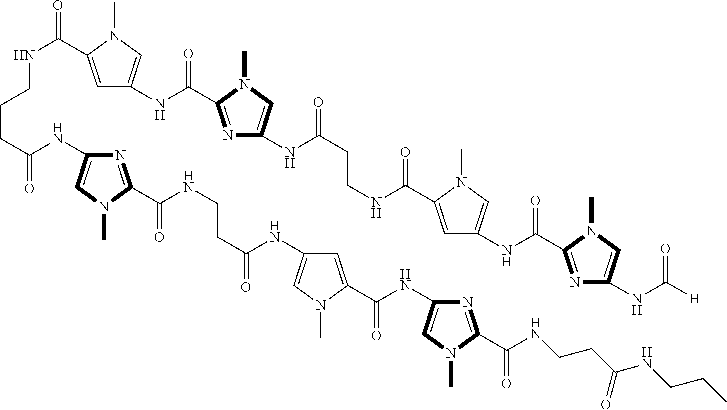 | B-324 |
| 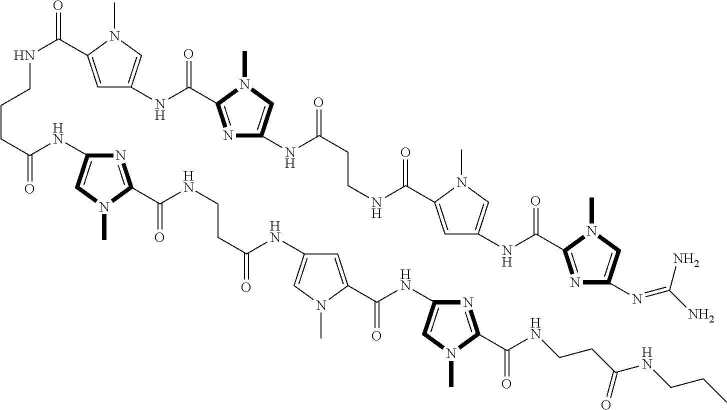 | B-325 |
| 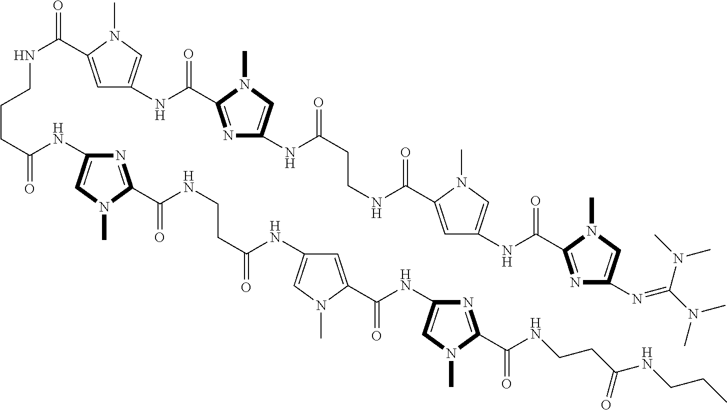 | B-326 |
| 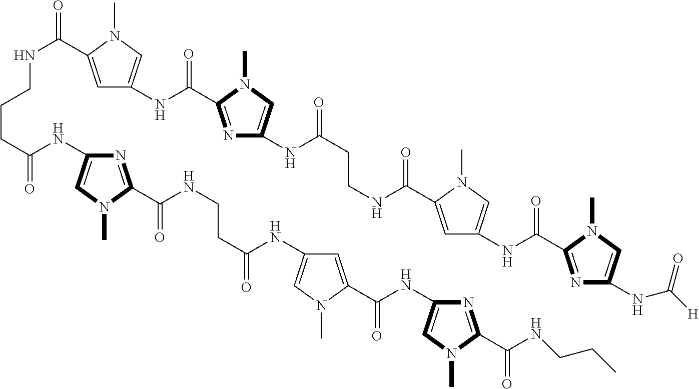 | B-327 |
| 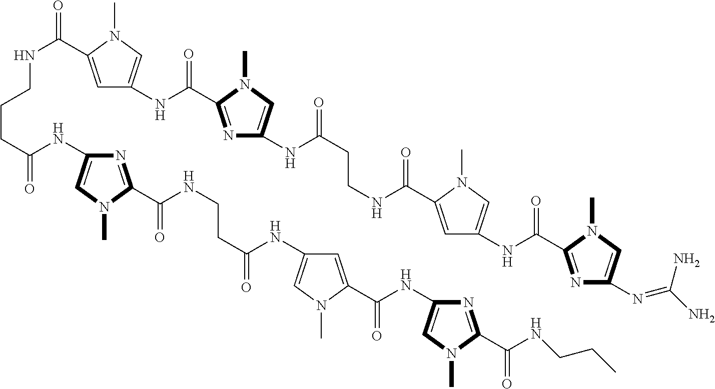 | B-328 |
| 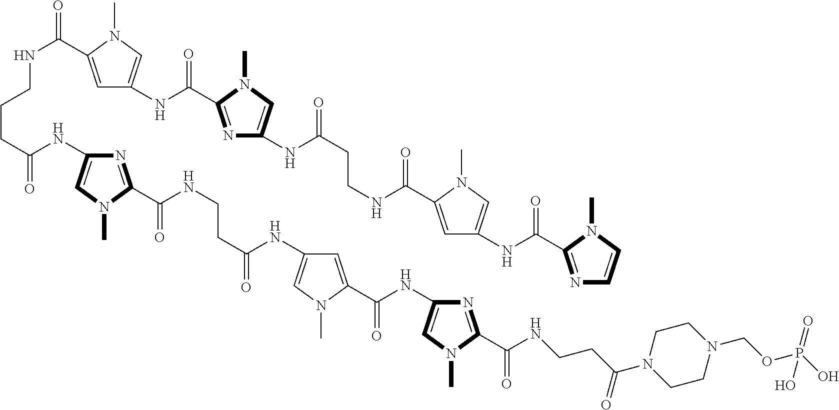 | B-330 |
| 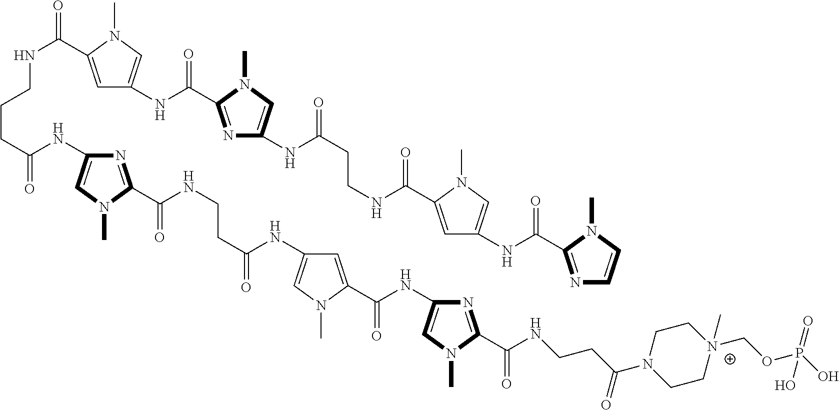 | B-331 |
| 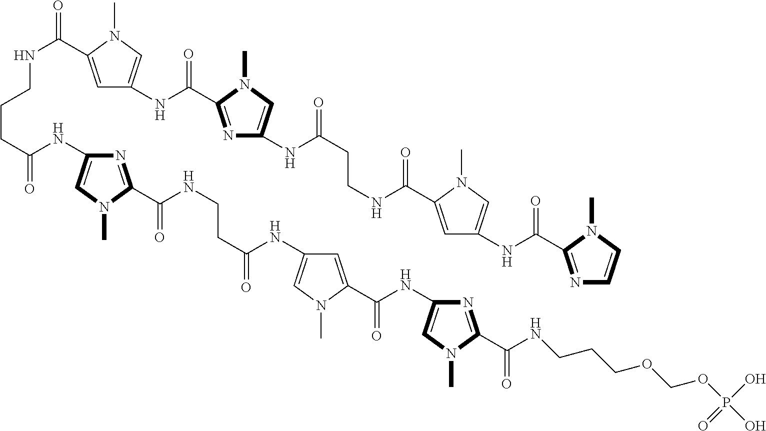 | B-332 |
| 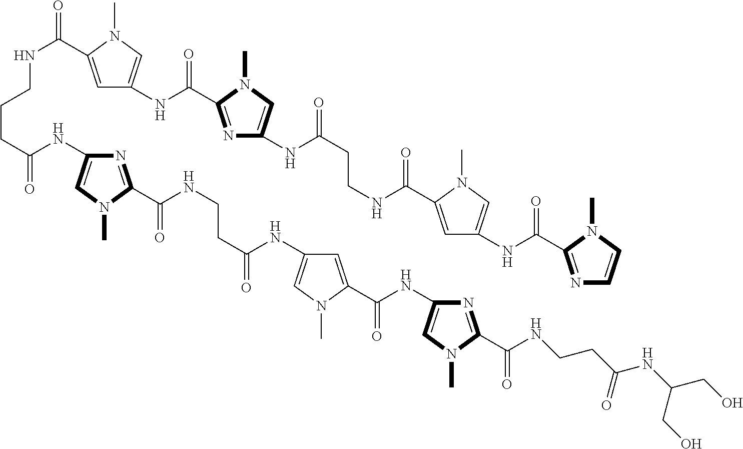 | B-333 |
| 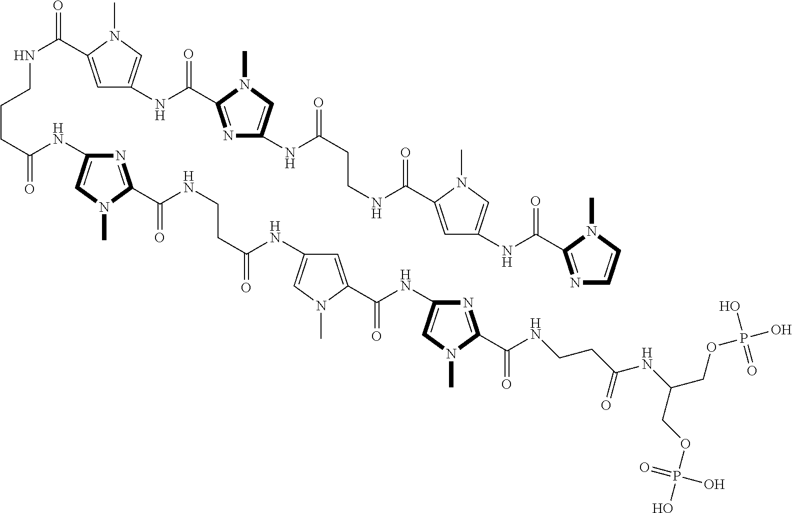 | B-334 |
| 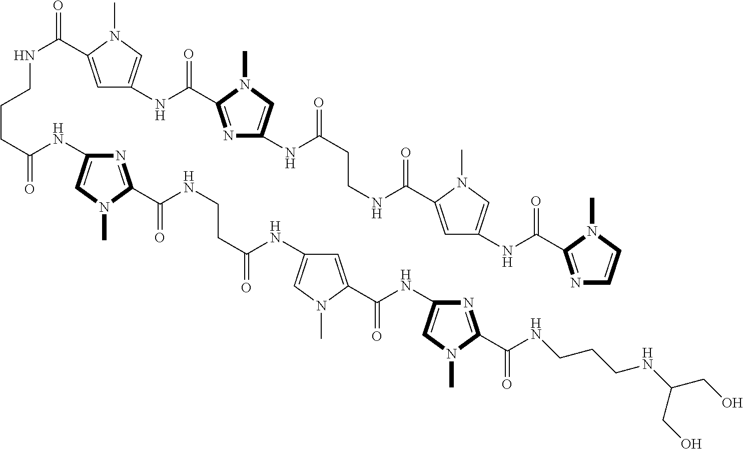 | B-335 |
| 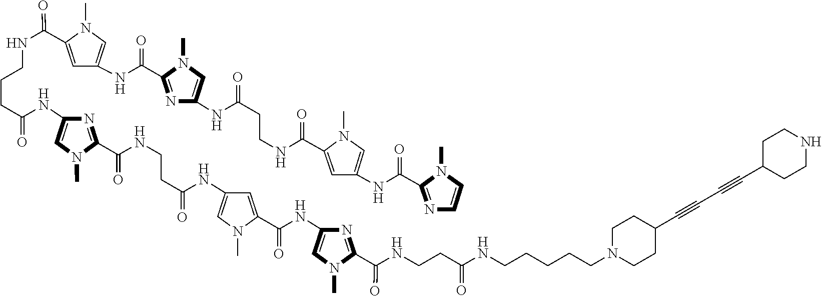 | B-336 |
| 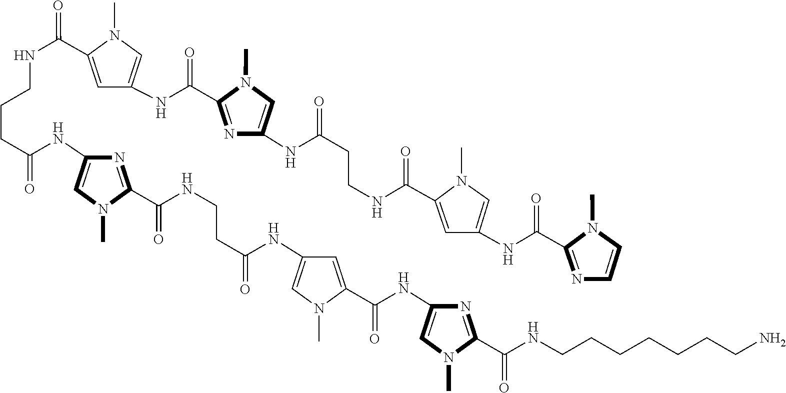 | B-337 |
| 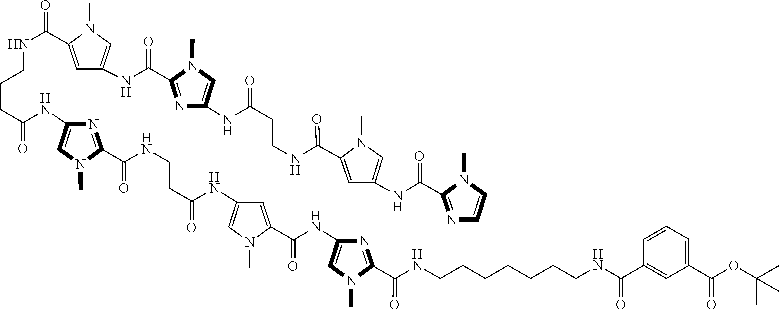 | B-338 |
| 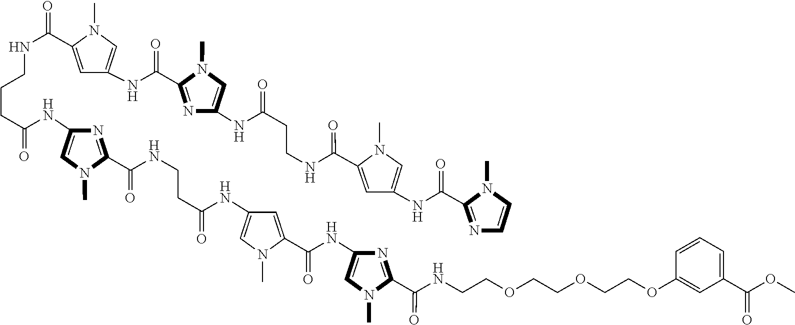 | B-339 |
| 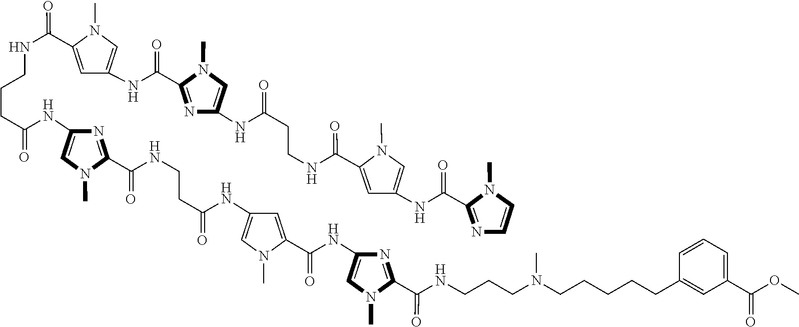 | B-340 |
| 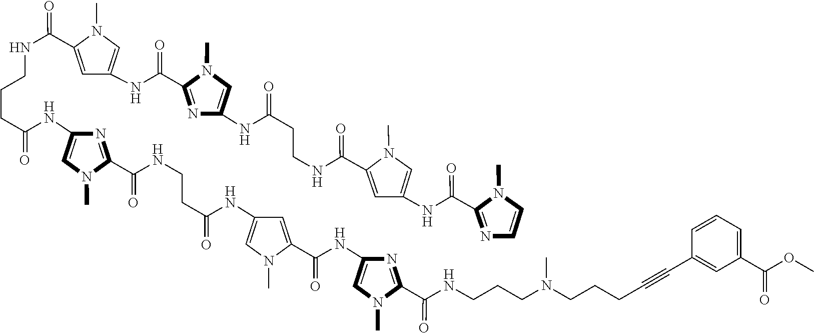 | B-341 |
| 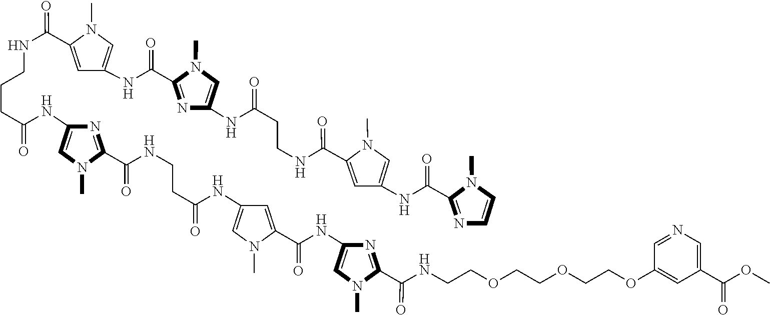 | B-342 |
| 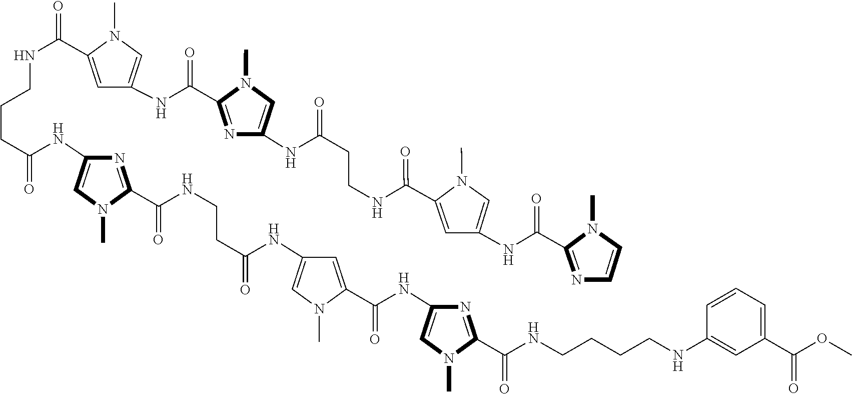 | B-343 |
| 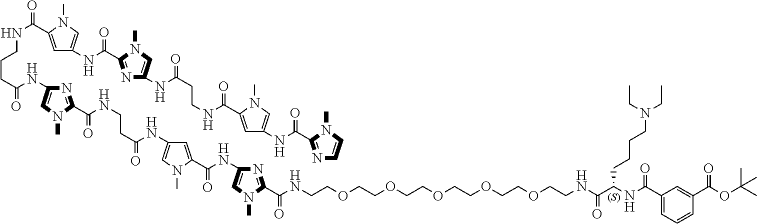 | B-344 |
| 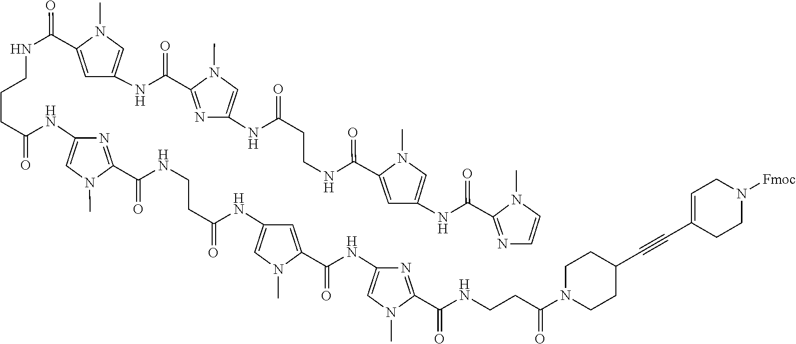 | B-345 |
| 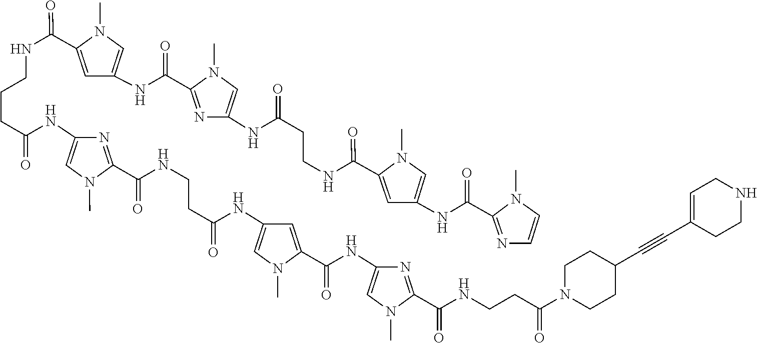 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)N4CCC(C#CC5=CCNCC5)CC4)n3)n(C)c2)n1] | B-346 |
| 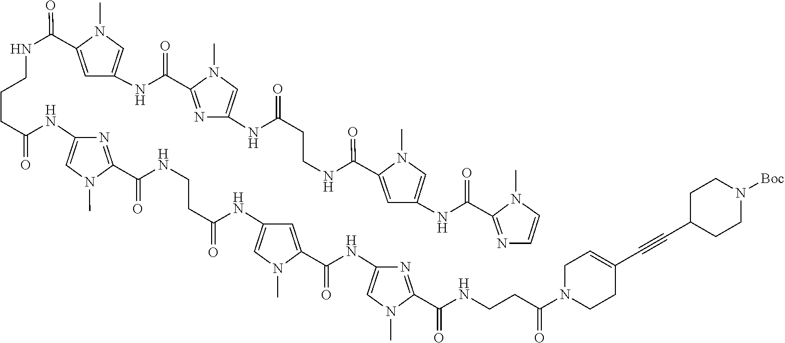 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)N4CC=C(C#CC5CCN(C(=O)OC(C)(C)C)CC5)CC4)n3)n(C)c2)n1] | B-347 |
| 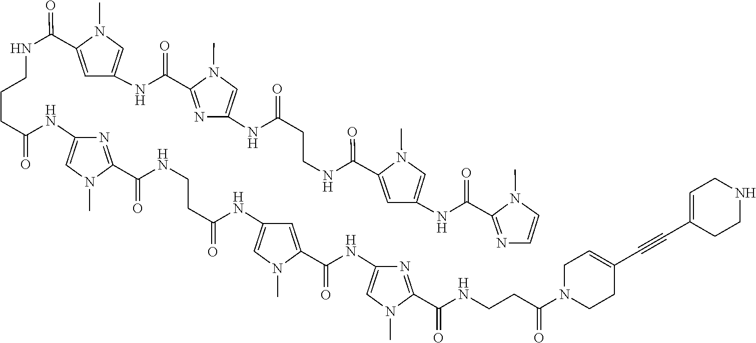 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)N4CC=C(C#CC5=CCNCC5)CC4)n3)n(C)c2)n1] | B-348 |
| 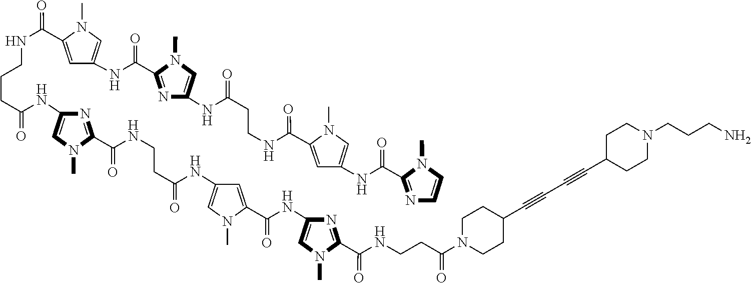 | B-349 |
| 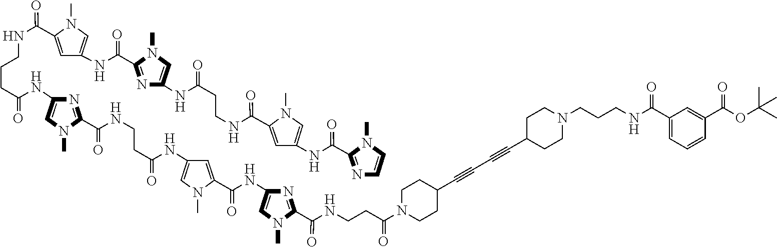 | B-350 |
| 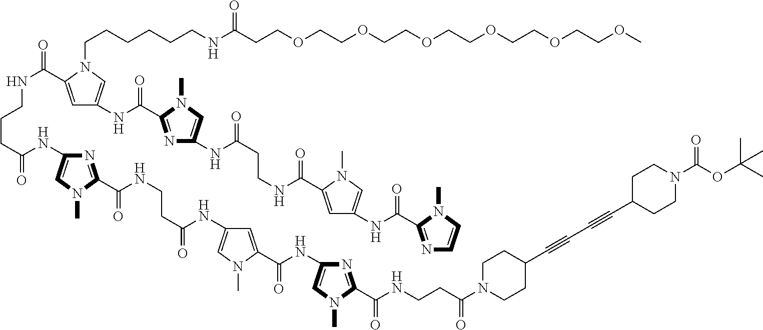 | B-351 |
| 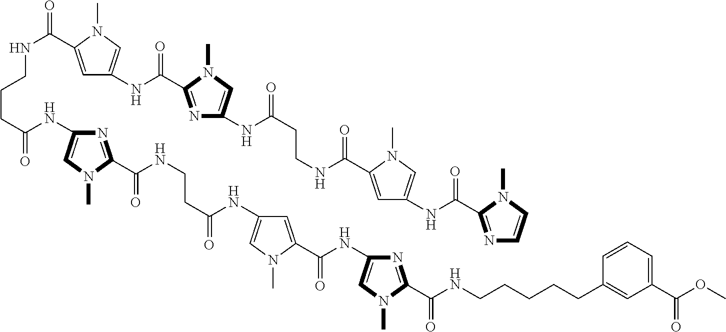 | B-352 |
| 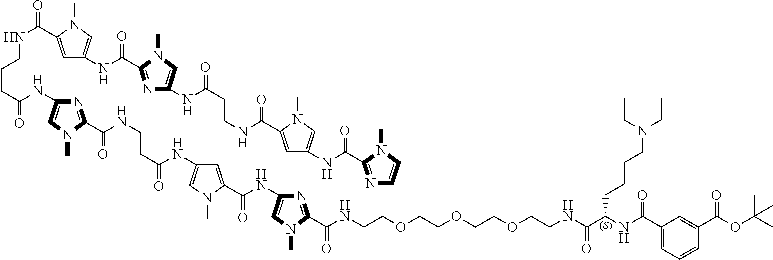 | B-353 |
| 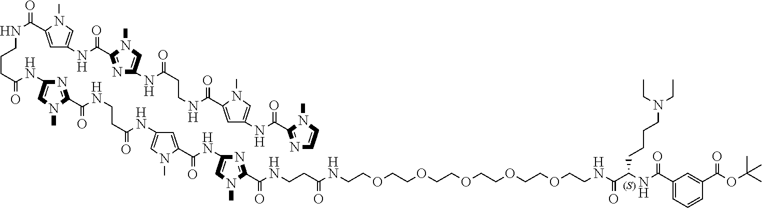 | B-354 |
| 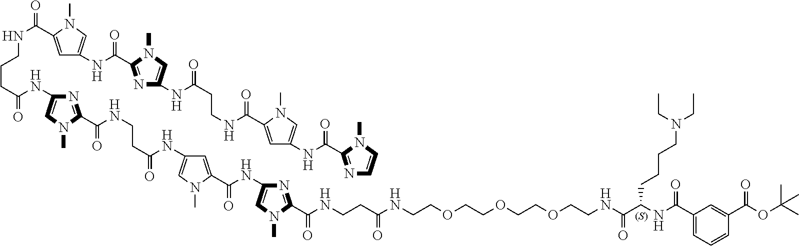 | B-355 |
| 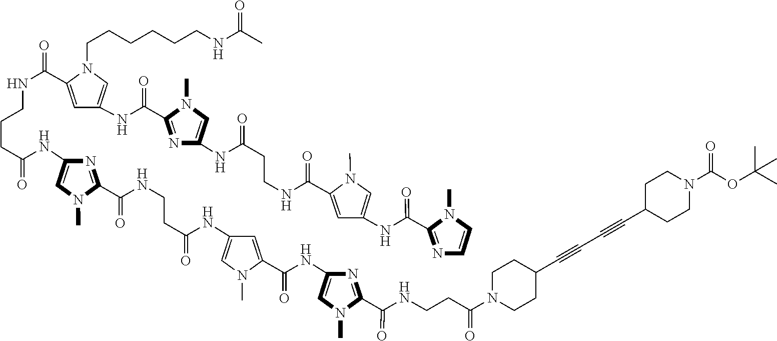 | B-356 |
| 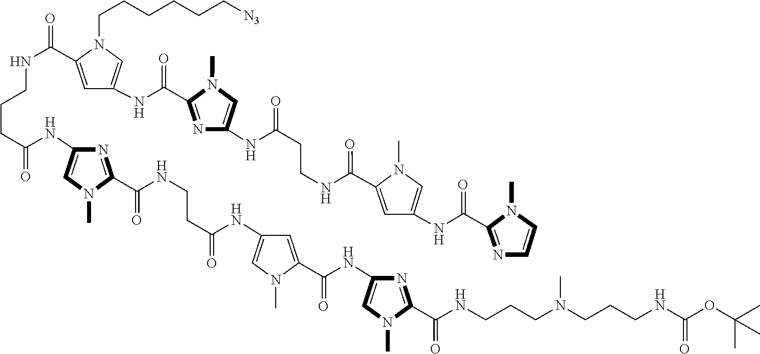 | B-357 |
| 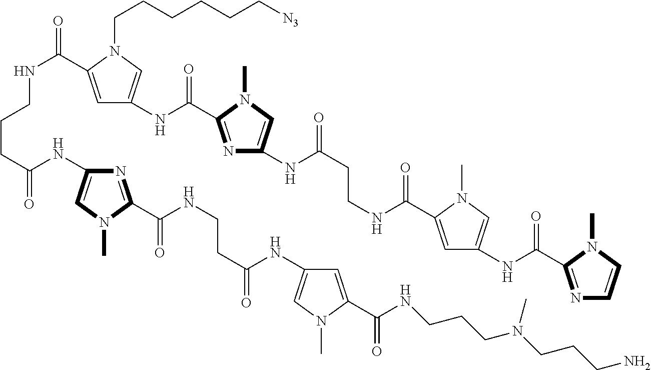 | B-358 |
| 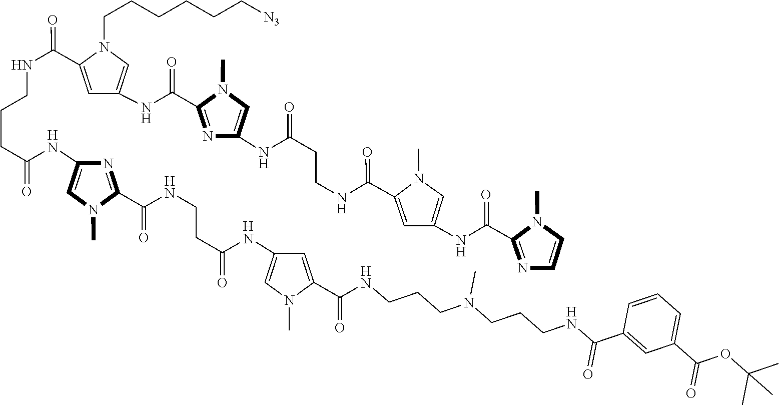 | B-359 |
| 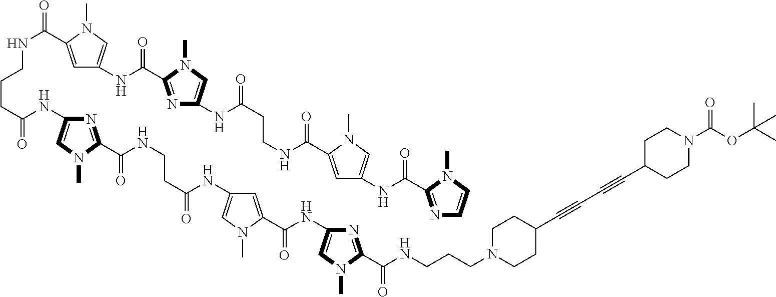 | B-360 |
| 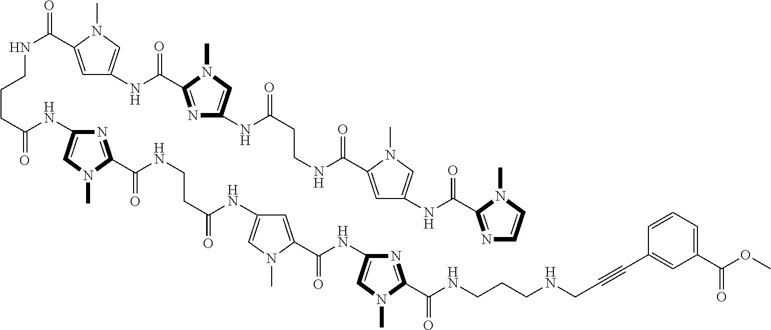 | B-361 |
| 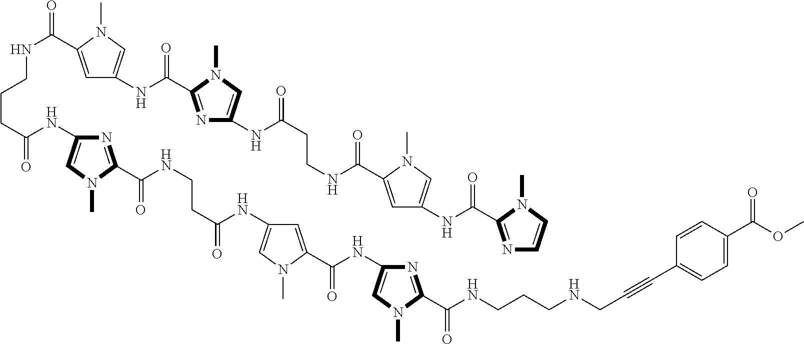 | B-362 |
| 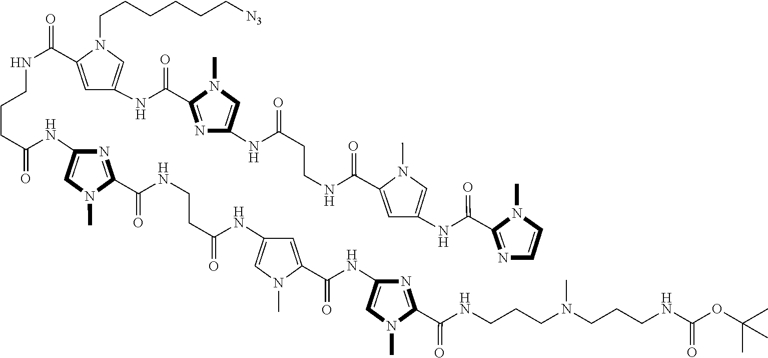 | B-363 |
| 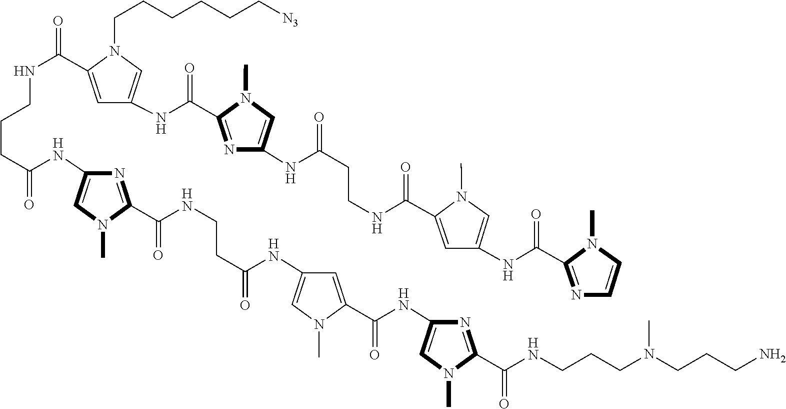 | B-364 |
| 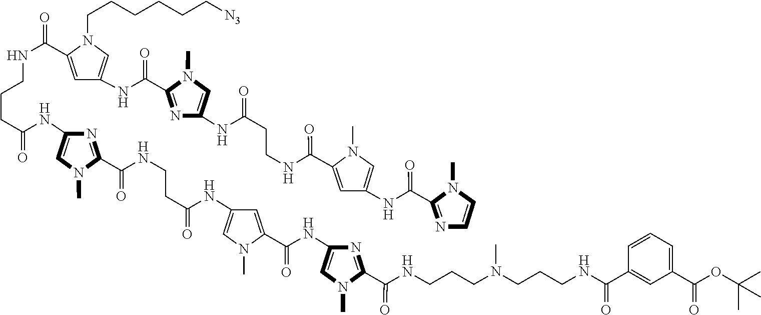 | B-365 |
| 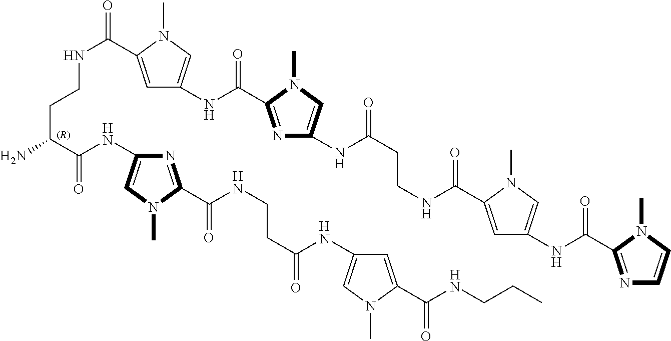 | B-366 |
| 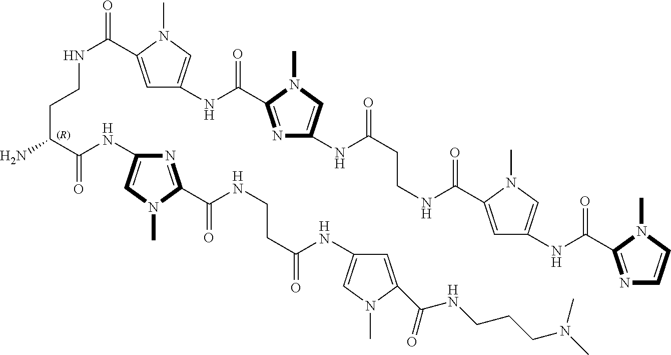 | B-367 |
| 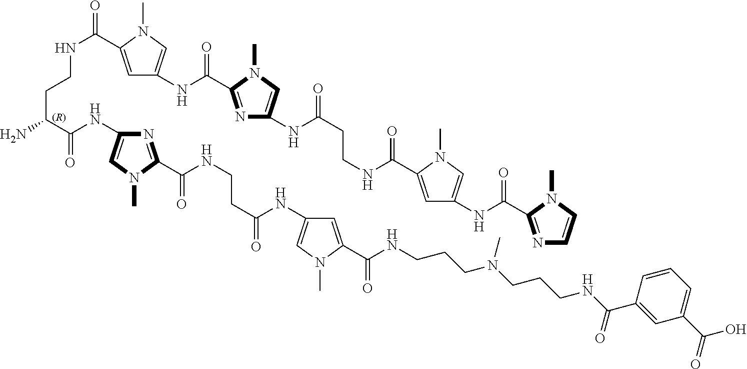 | B-368 |
| 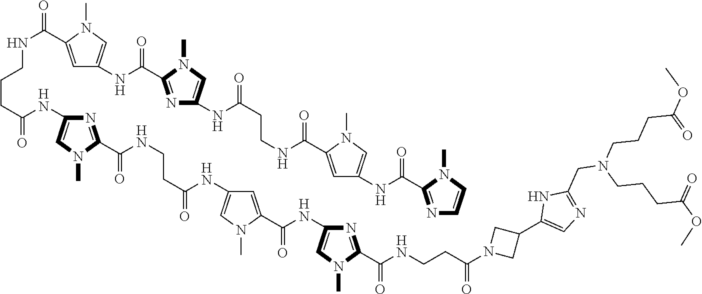 | B-369 |
| 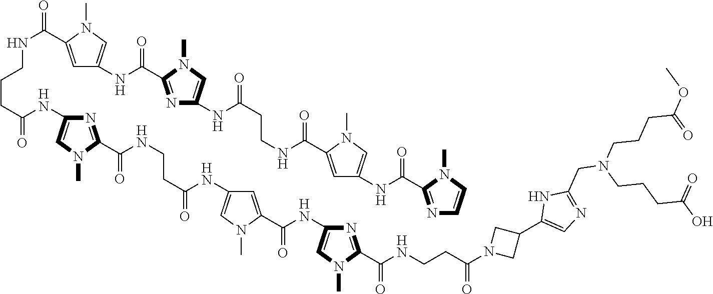 | B-370 |
| 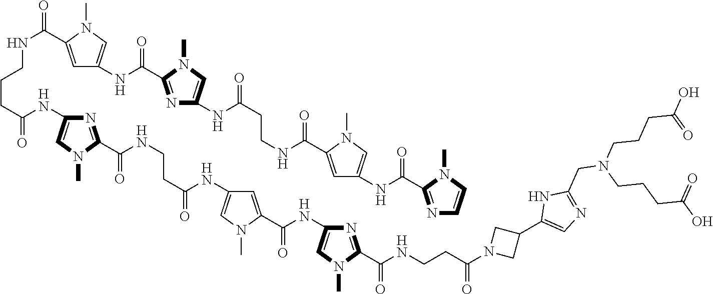 | B-371 |
| 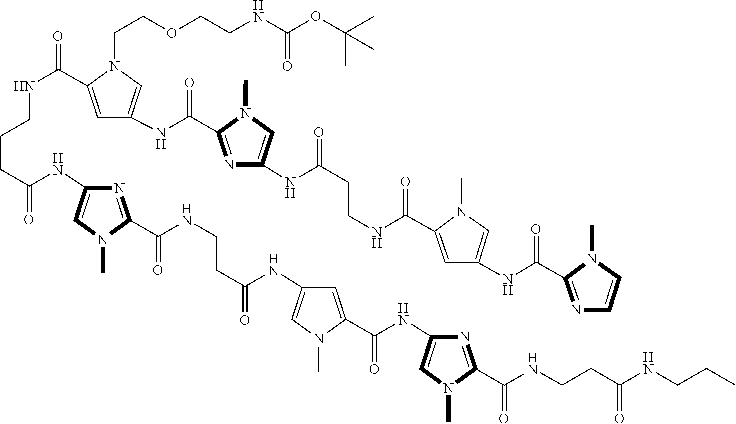 | B-372 |
| 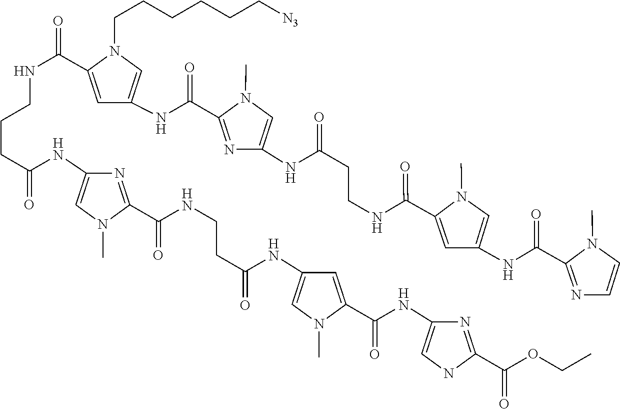 [SMILES: CCOC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4CCCCCCN=[N+]=[N-])cn3C)cn2C)c[nH]1] | B-373 |
| 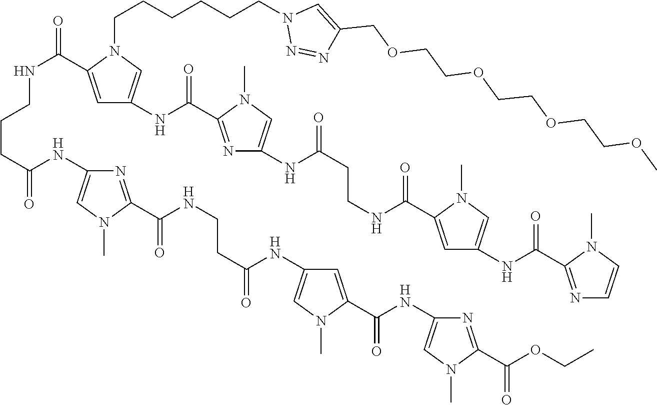 [SMILES: CCOC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4CCCCCCn4cc(COCCOCCOCCOC)nn4)cn3C)cn2C)cn1C] | B-374 |
| 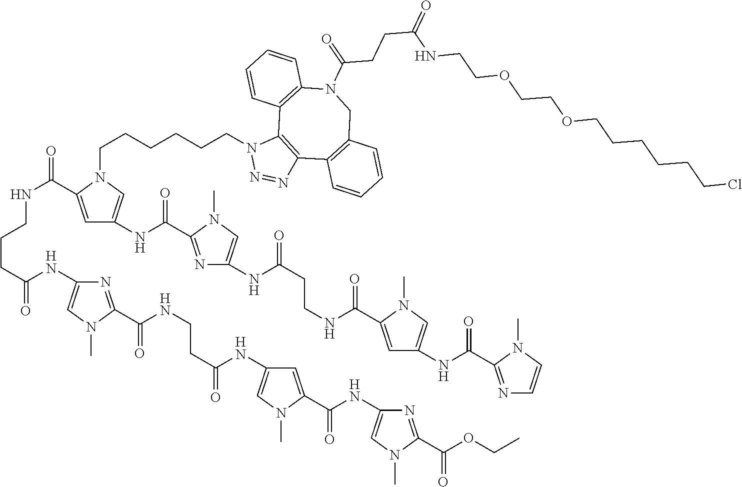 [SMILES: CCOC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4CCCCCCn4nnc5c4-c4ccccc4N(C(=O)CCC(=O)NCCOCCOCCCCCCCl)Cc4ccccc4-5)cn3C)cn2C)cn1C] | B-375 |
| 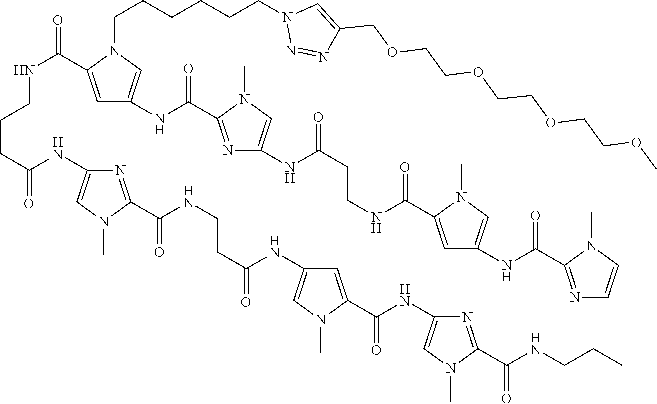 [SMILES: CCCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4CCCCCCn4cc(COCCOCCOCCOC)nn4)cn3C)cn2C)cn1C] | B-376 |
| 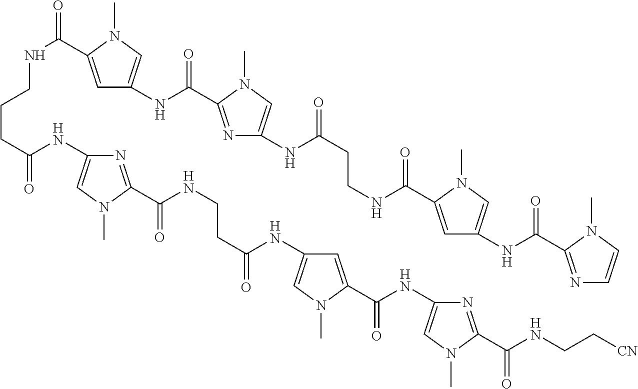 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC#N)n3)n(C)c2)n1] | B-377 |
| 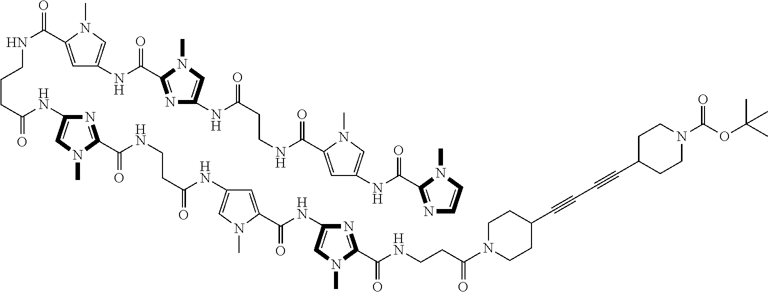 | B-378 |
| 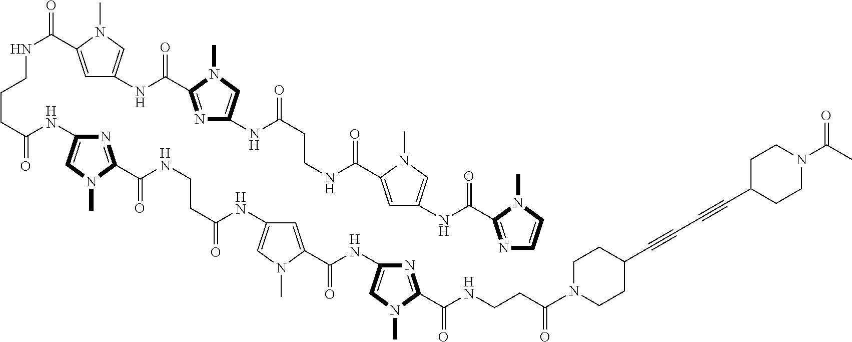 | B-379 |
| 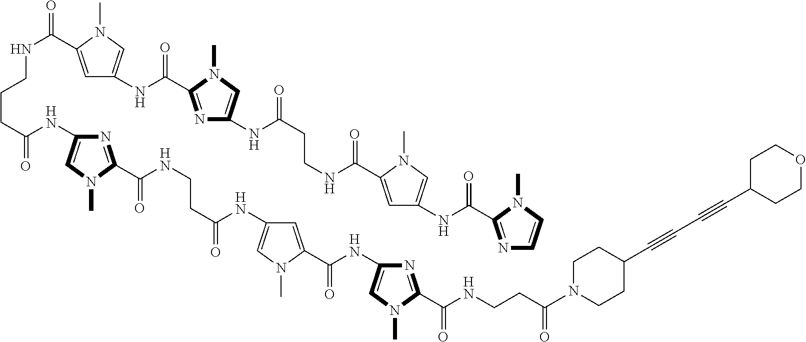 | B-380 |
| 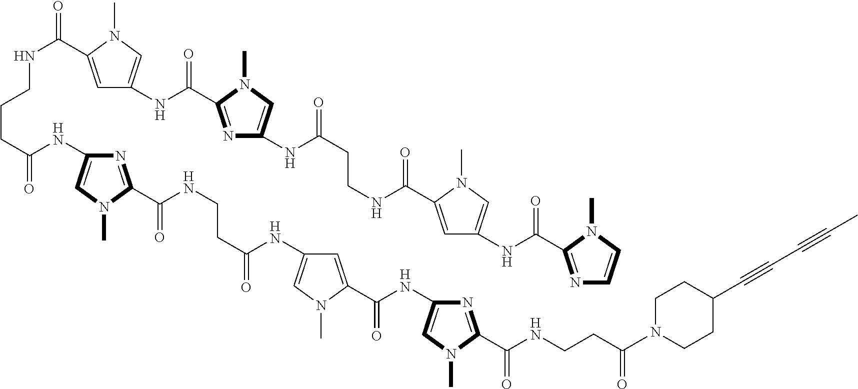 | B-381 |
| 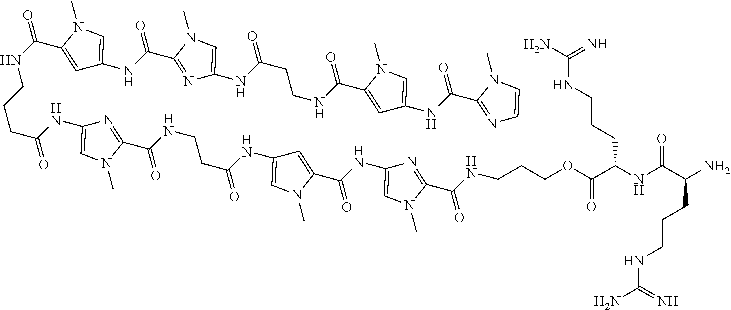 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCCOC(=O)[C@H](CCCNC(=N)N)NC(=O)[C@@H](N)CCCNC(=N)N)n3)n(C)c2)n1] | B-382 |
| 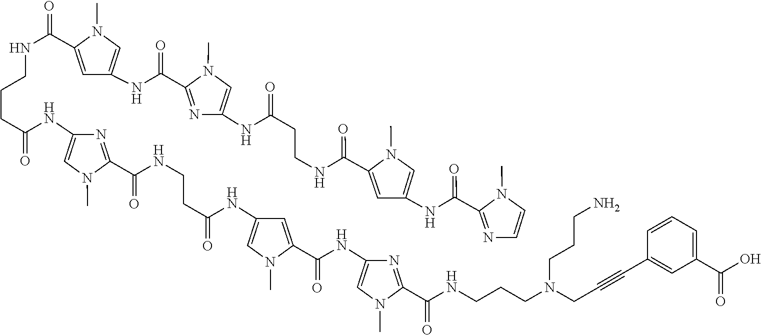 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCCN(CC#Cc4cccc(C(=O)O)c4)CCCN)n3)n(C)c2)n1] | B-383 |
| 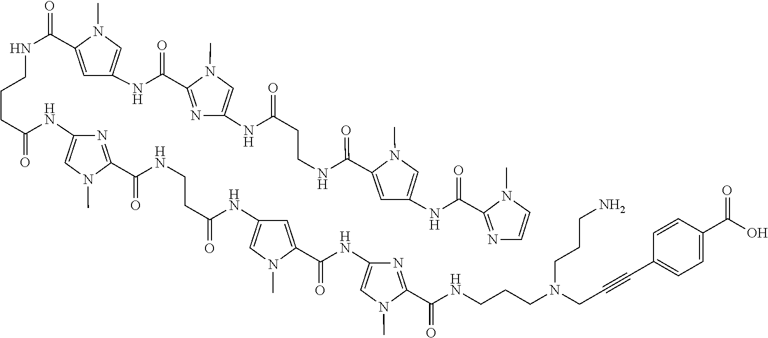 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCCN(CC#Cc4ccc(C(=O)O)cc4)CCCN)n3)n(C)c2)n1] | B-384 |
| 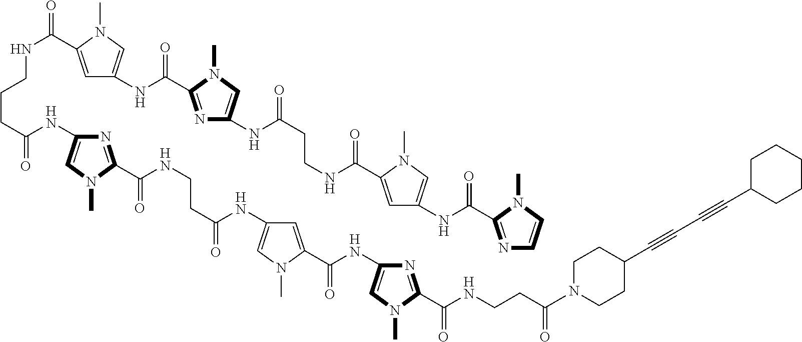 | B-385 |
| 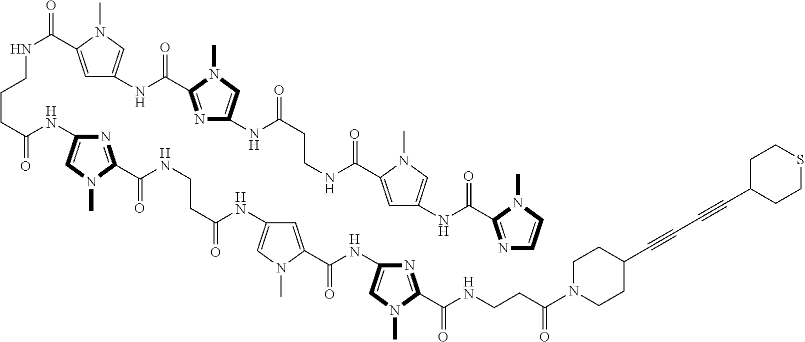 | B-386 |
| 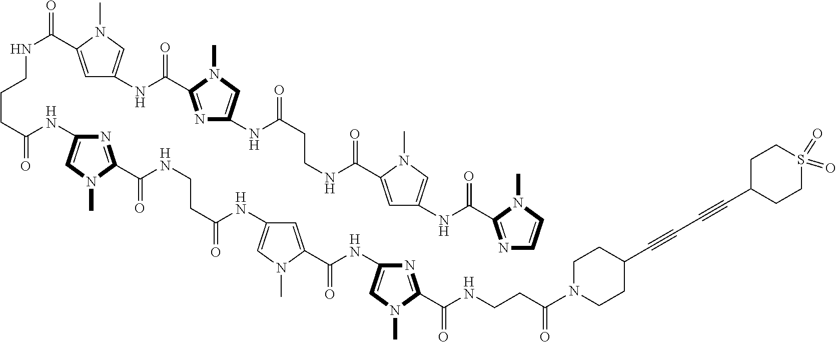 | B-387 |
| 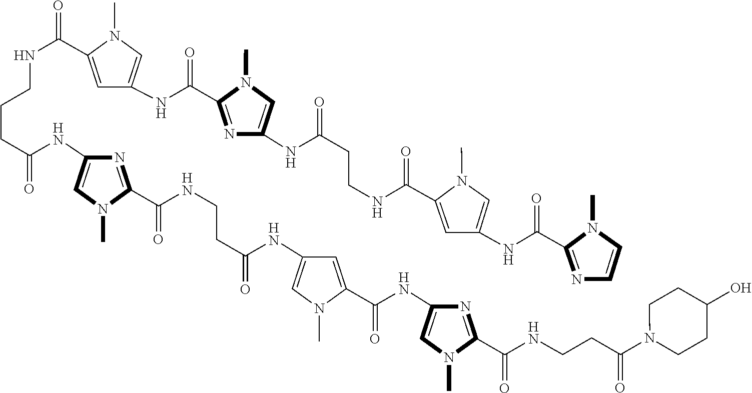 | B-388 |
| 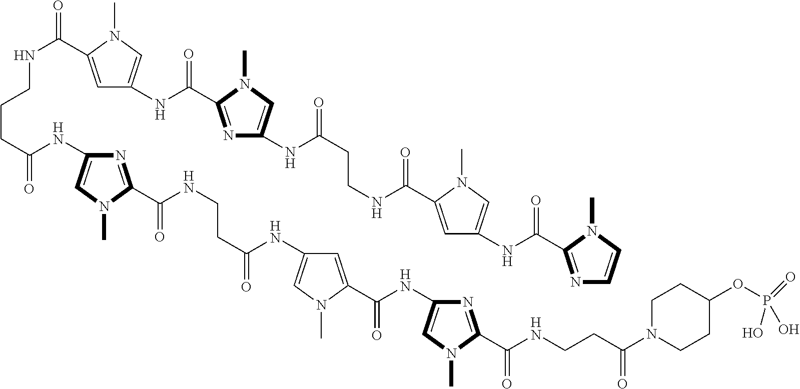 | B-389 |
| 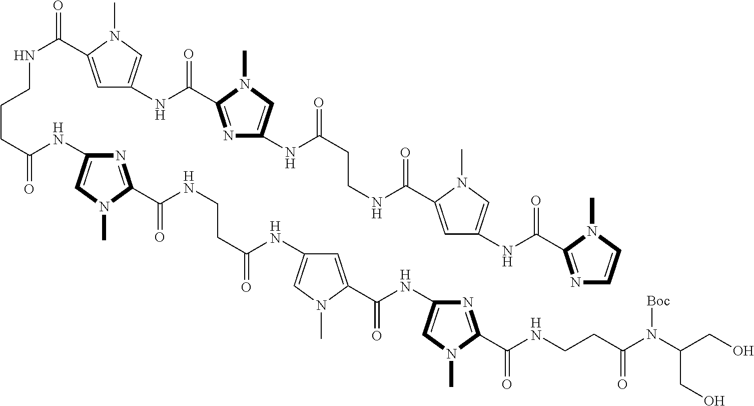 | B-390 |
| 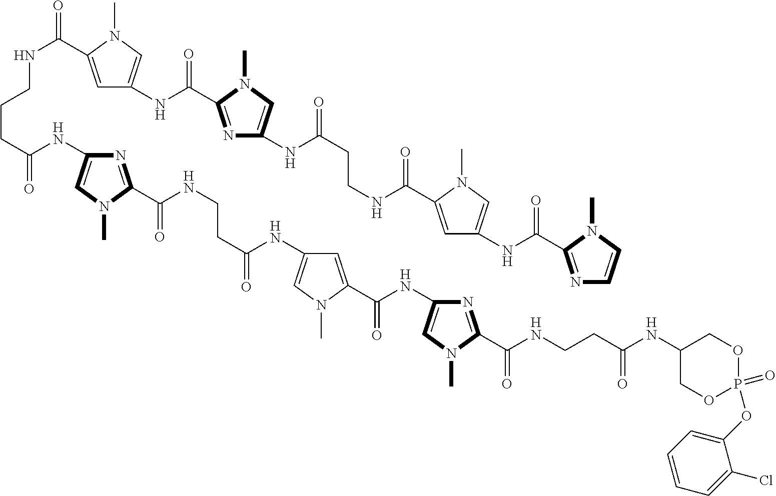 | B-391 |
| 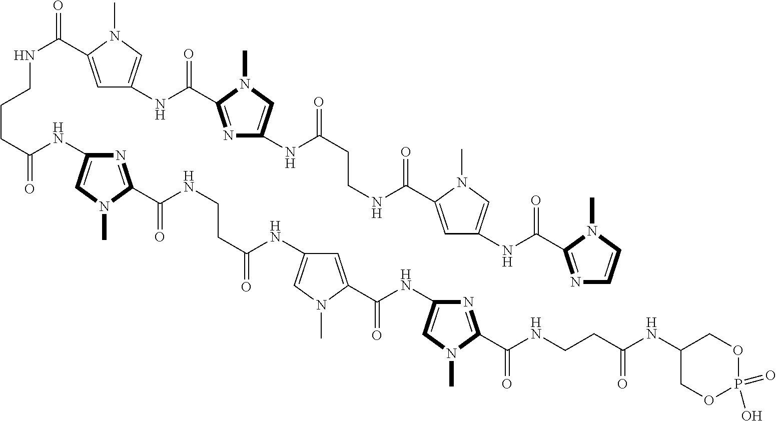 | B-392 |
| 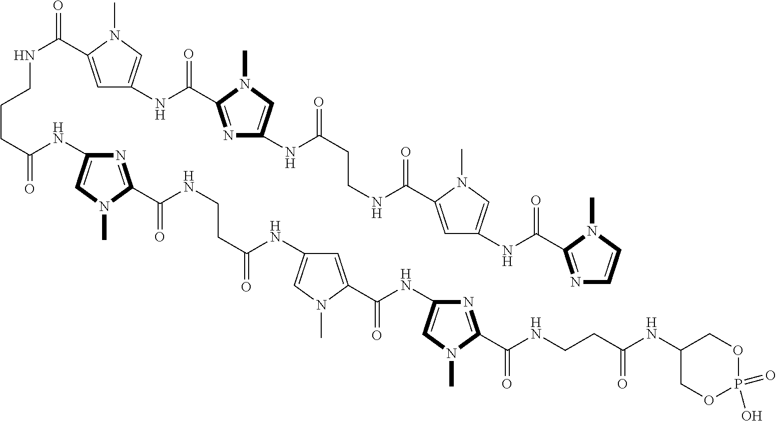 | B-393 |
| 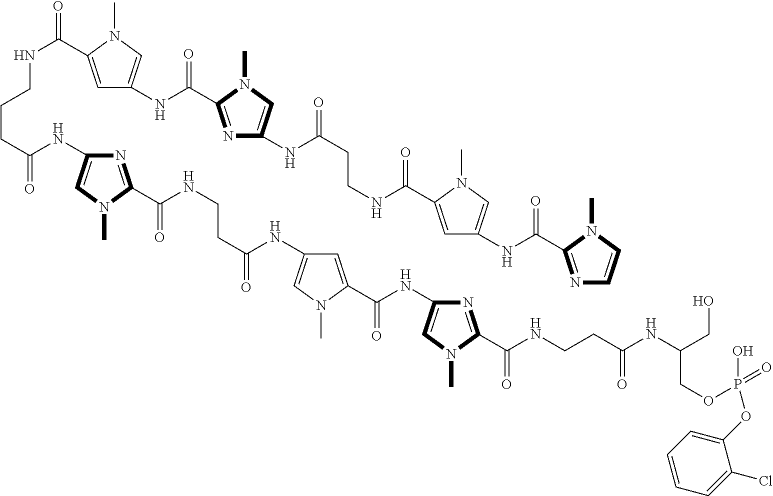 | B-394 |
| 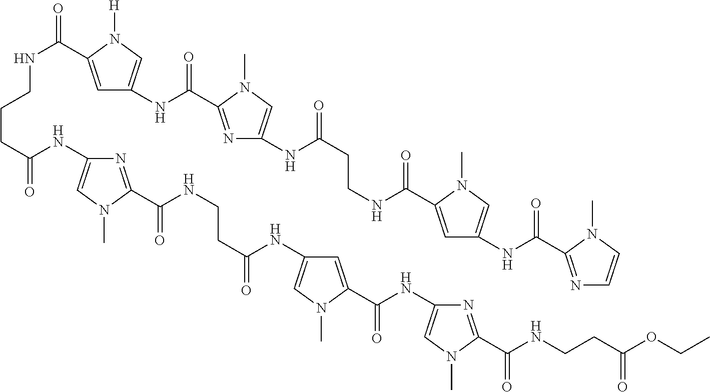 [SMILES: CCOC(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)c[nH]4)cn3C)cn2C)cn1C] | B-395 |
| 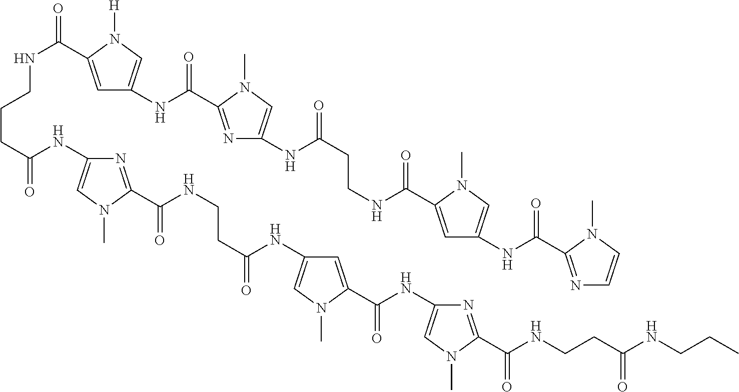 [SMILES: CCCNC(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)c[nH]4)cn3C)cn2C)cn1C] | B-396 |
| 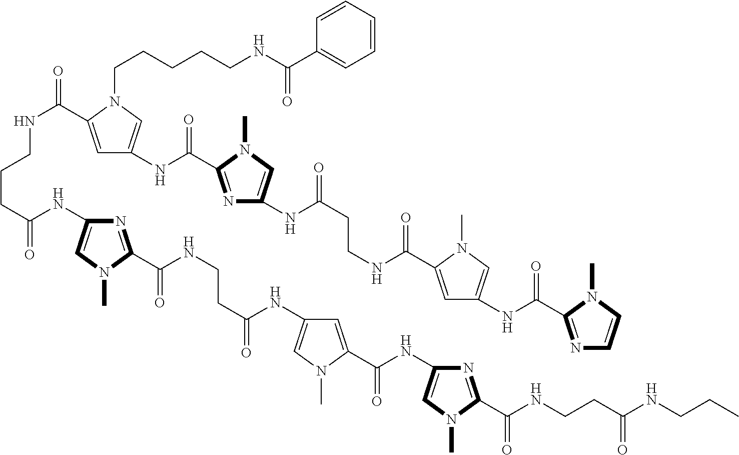 | B-397 |
| 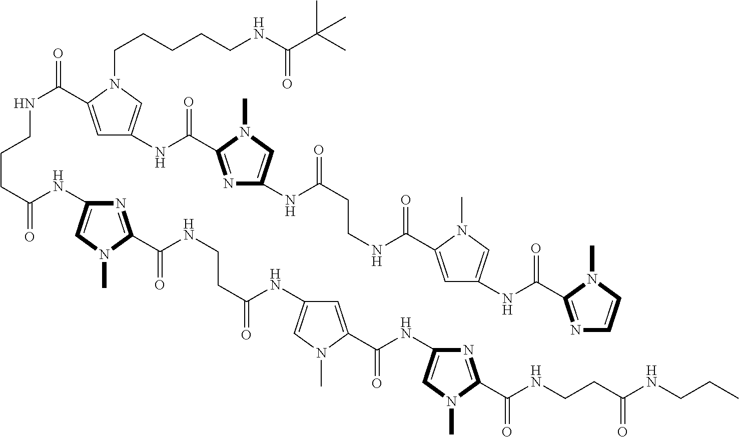 | B-398 |
| 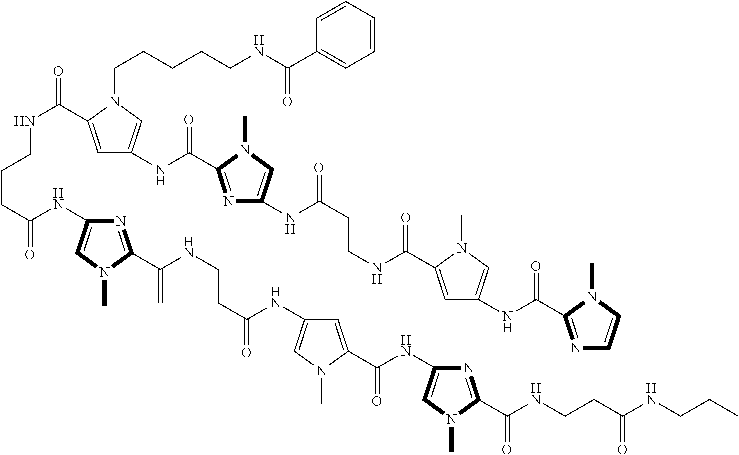 | B-399 |
| 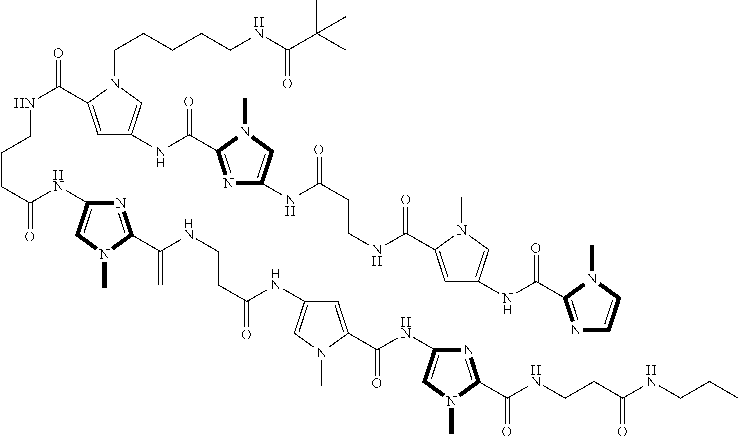 | B-400 |
| 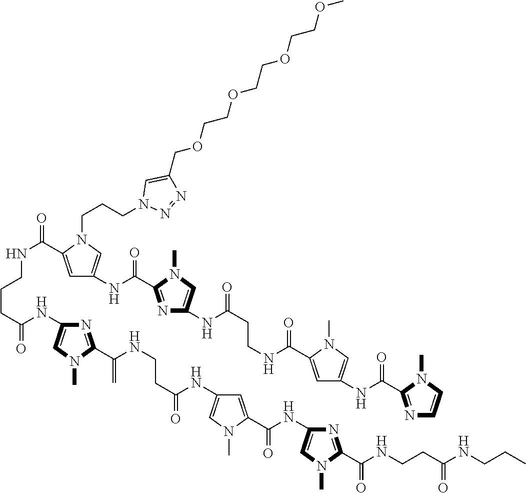 | B-401 |
| 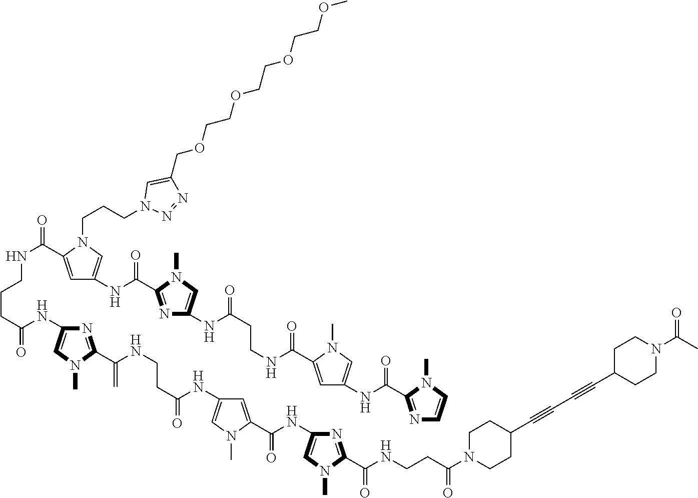 | B-402 |
| 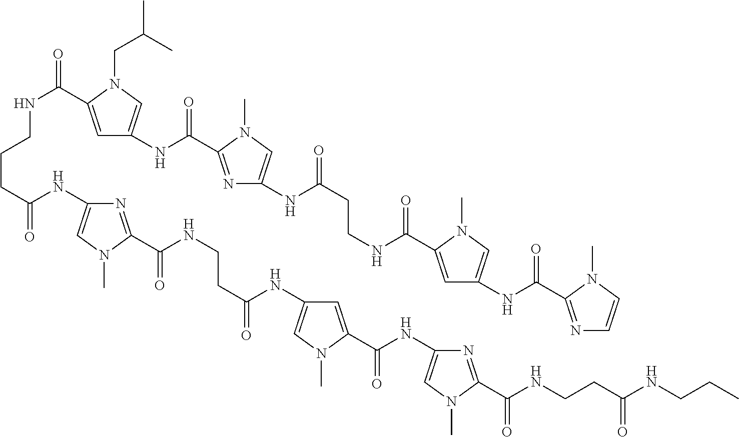 [SMILES: CCCNC(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4CC(C)C)cn3C)cn2C)cn1C] | B-403 |
| 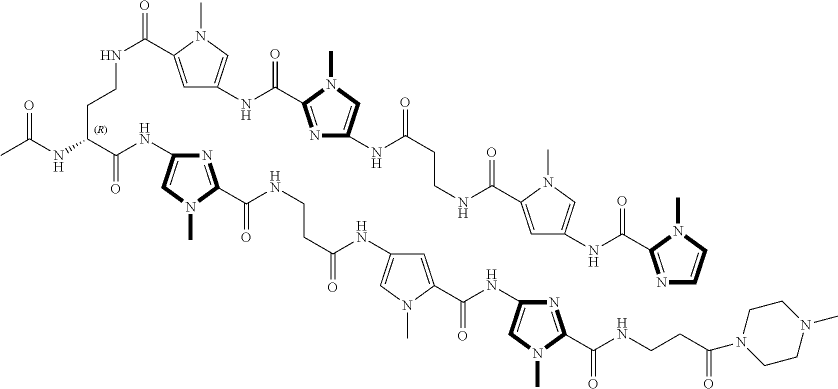 | B-404 |
| 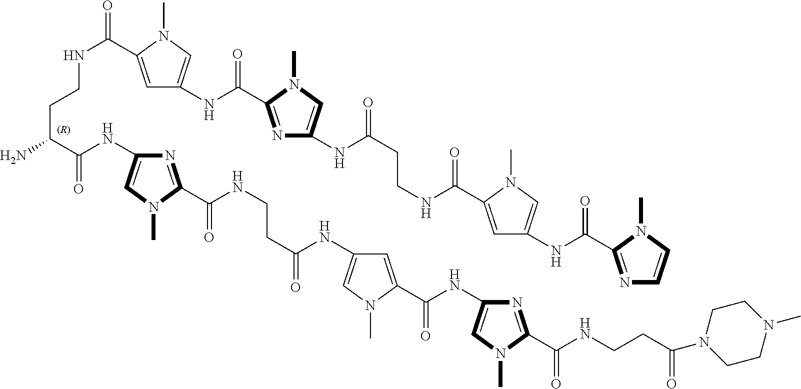 | B-405 |
| 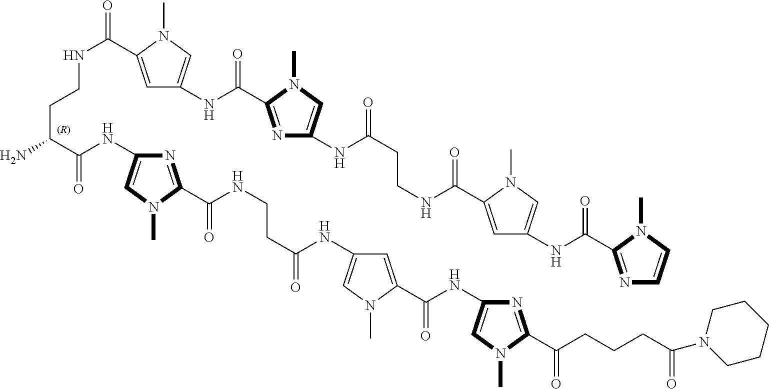 | B-406 |
| 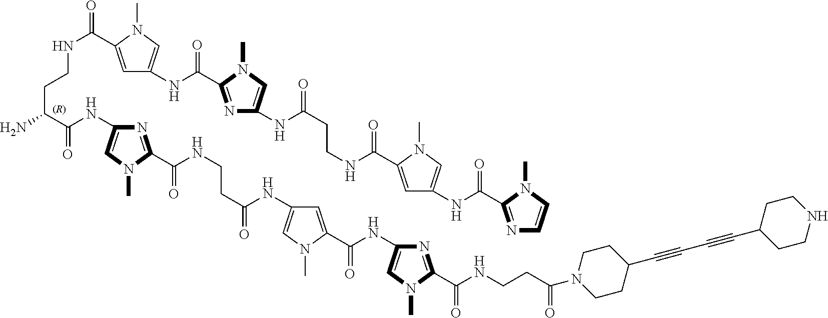 | B-407 |
| 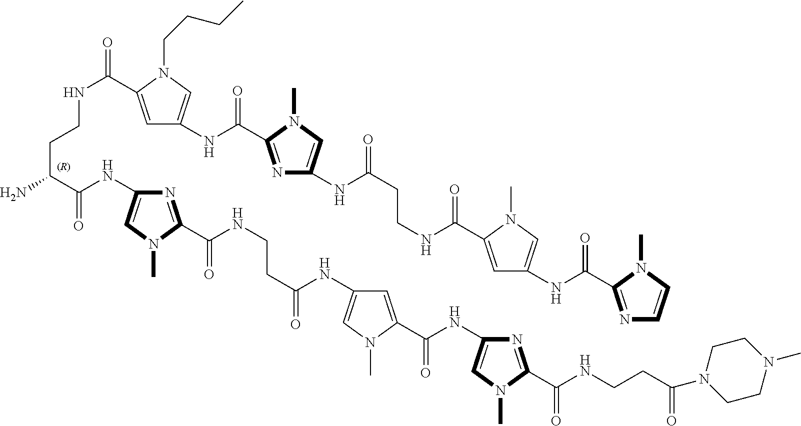 | B-408 |
| 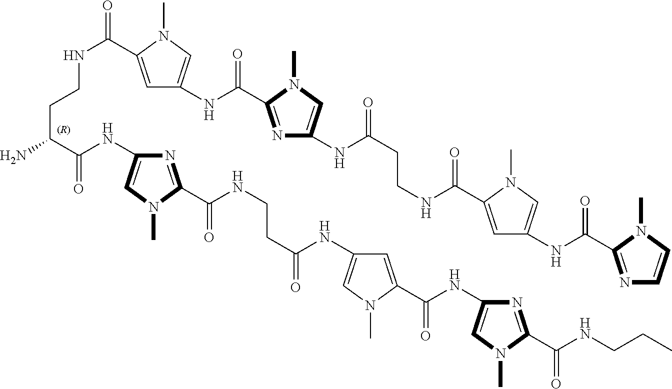 | B-409 |
| 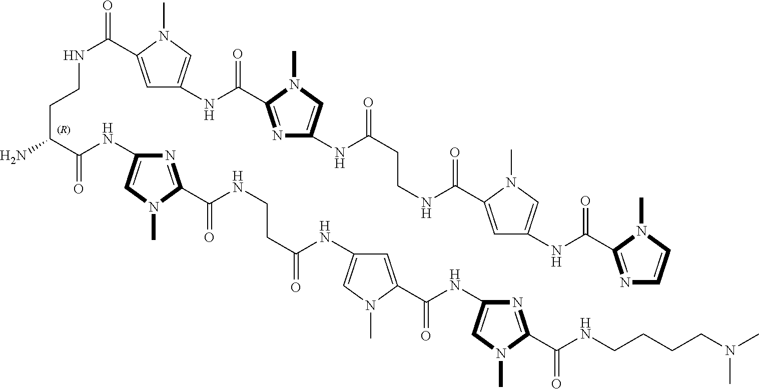 | B-410 |
| 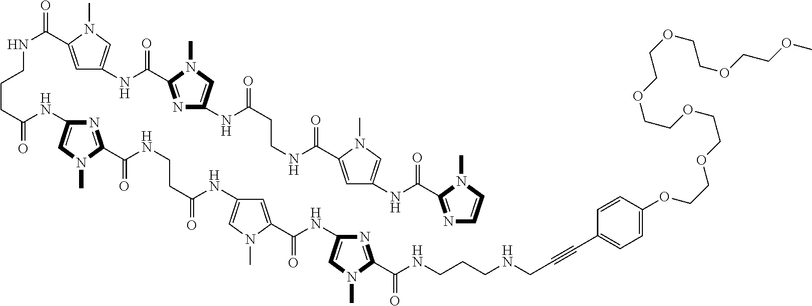 | B-411 |
| 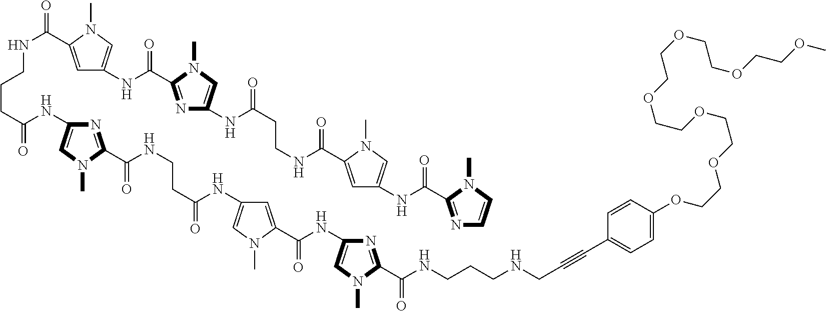 | B-412 |
| 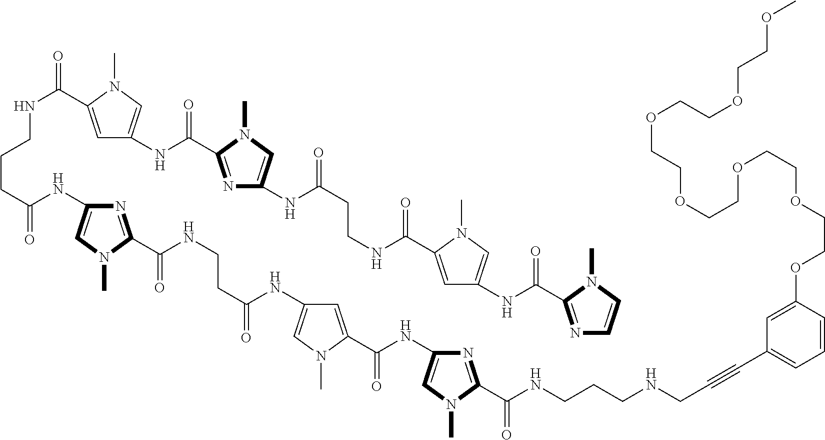 | B-413 |
| 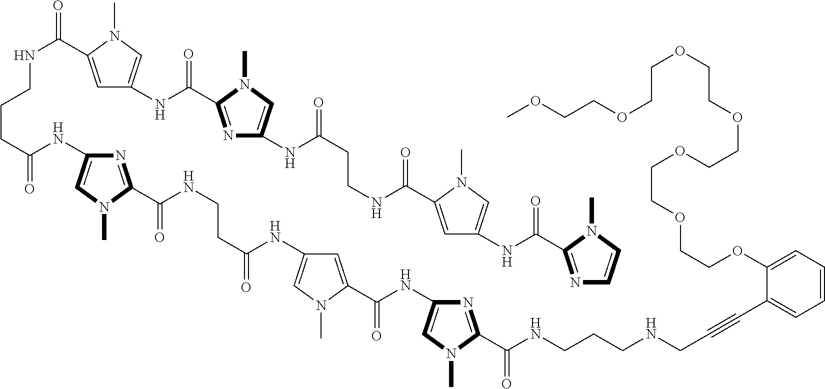 | B-414 |
| 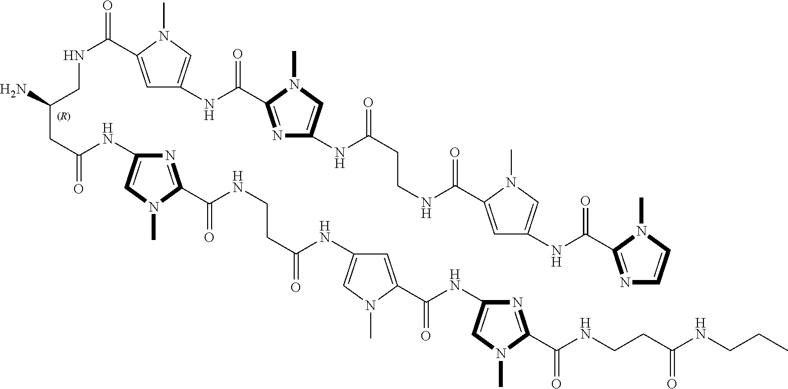 | B-415 |
| 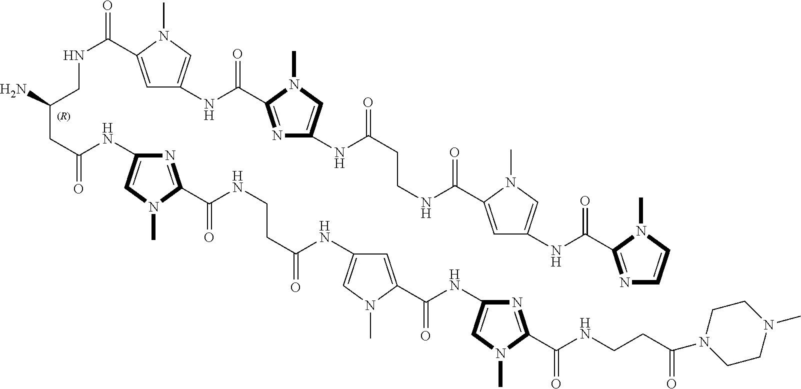 | B-416 |
| 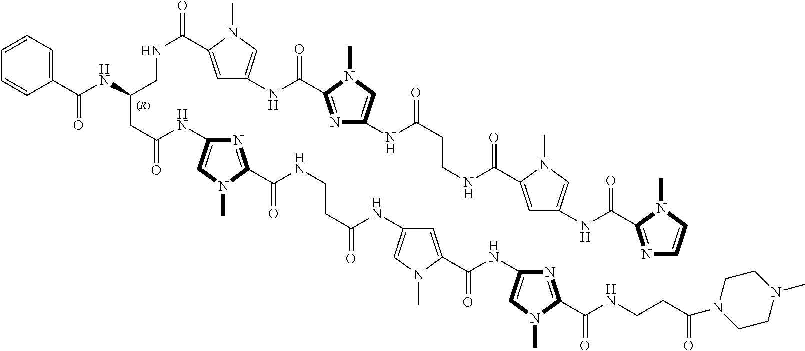 | B-417 |
| 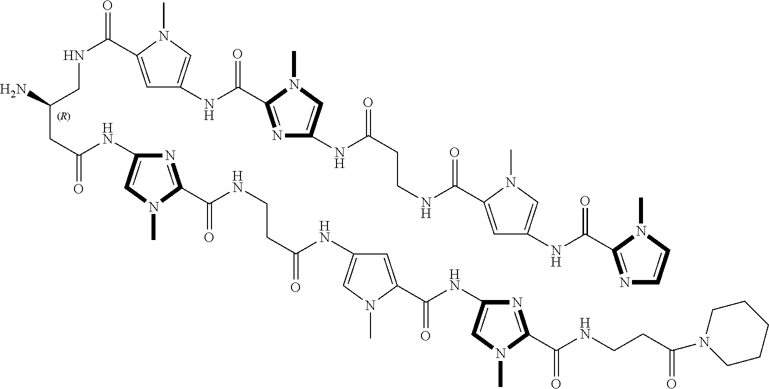 | B-418 |
| 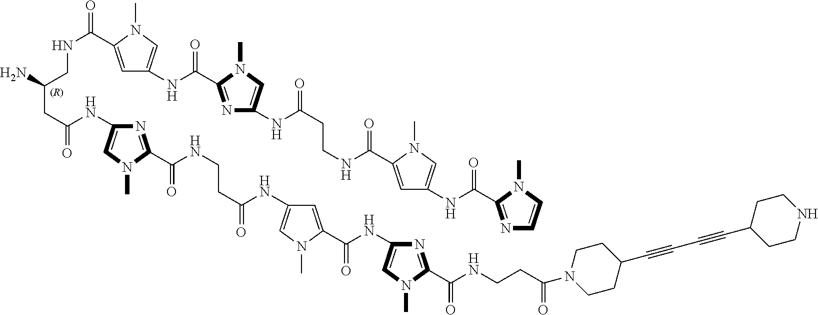 | B-419 |
| 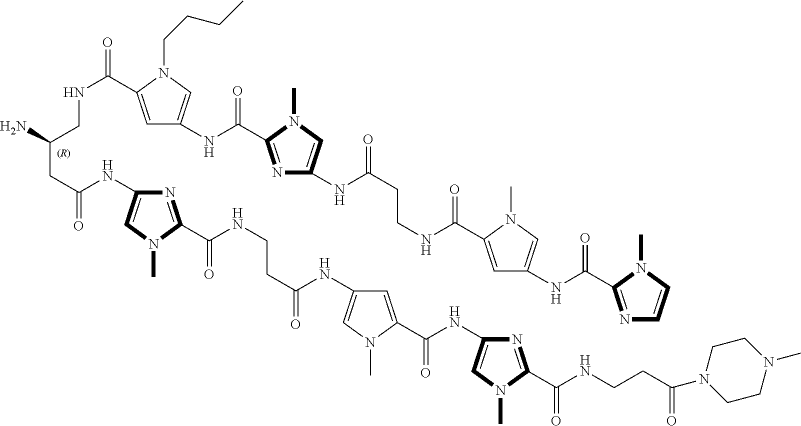 | B-420 |
| 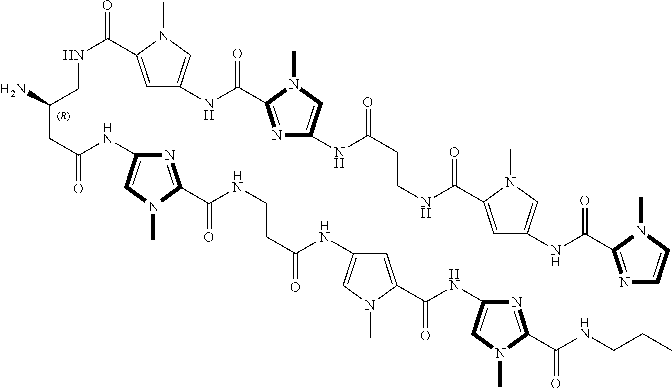 | B-421 |
| 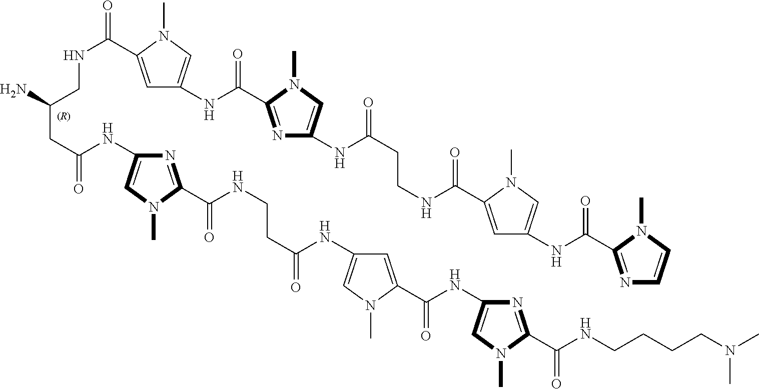 | B-422 |
| 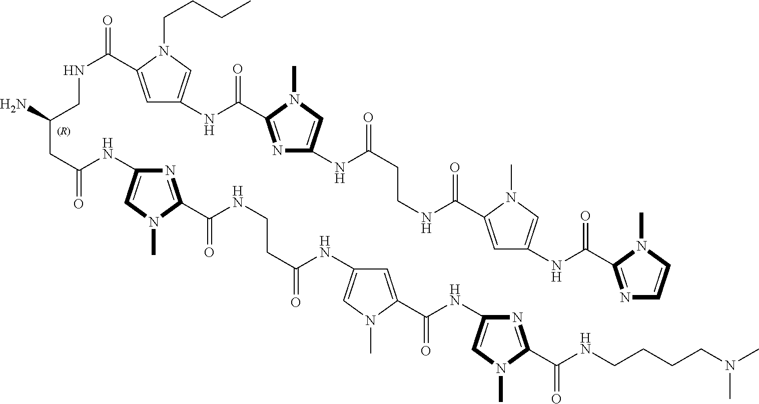 | B-423 |
| 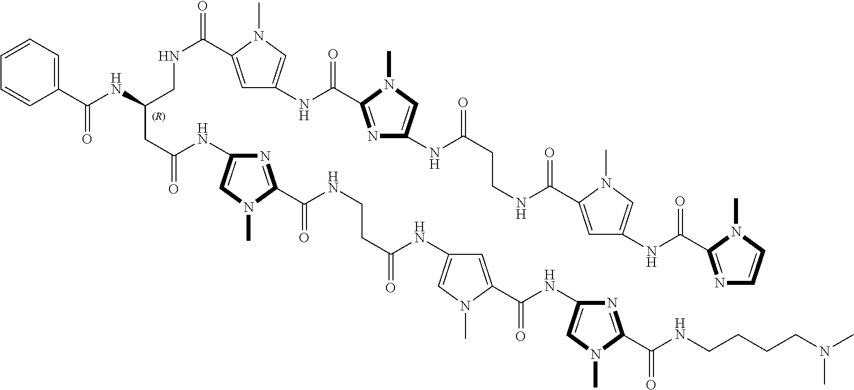 | B-424 |
| 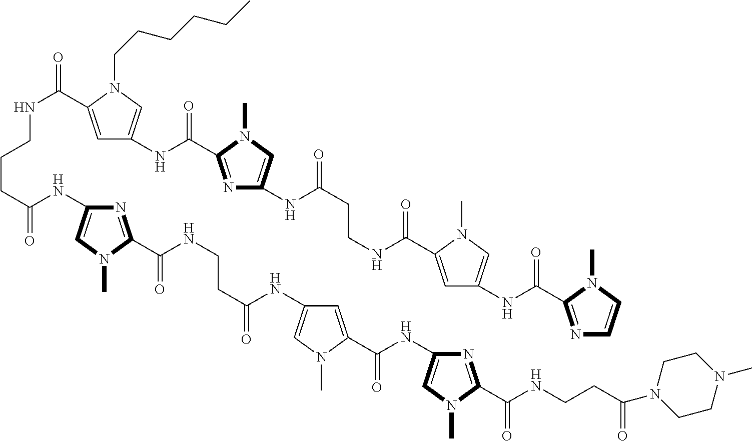 | B-425 |
| 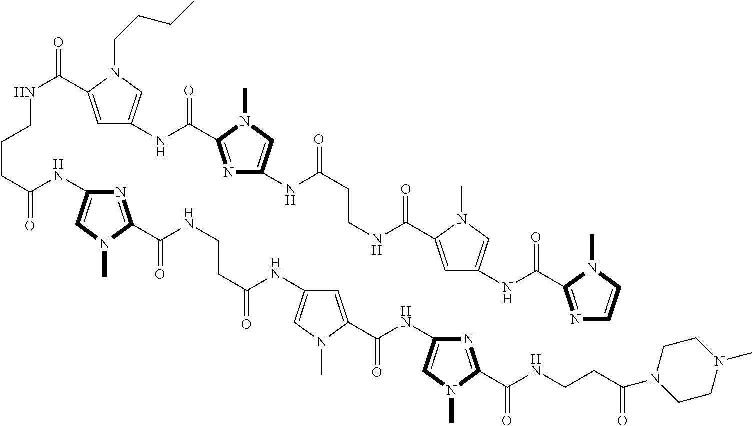 | B-426 |
| 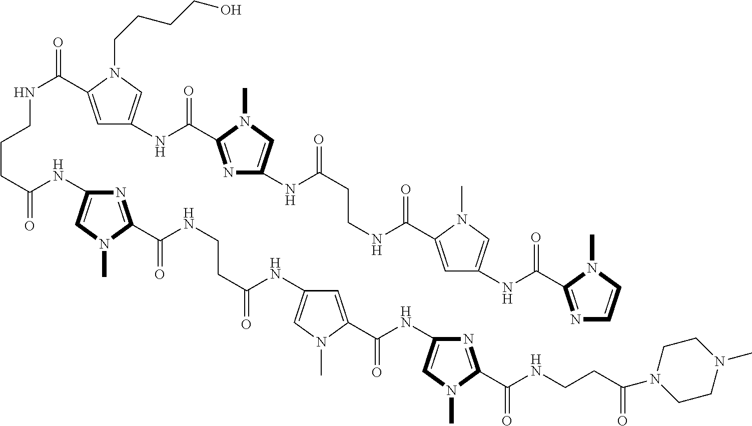 | B-427 |
| 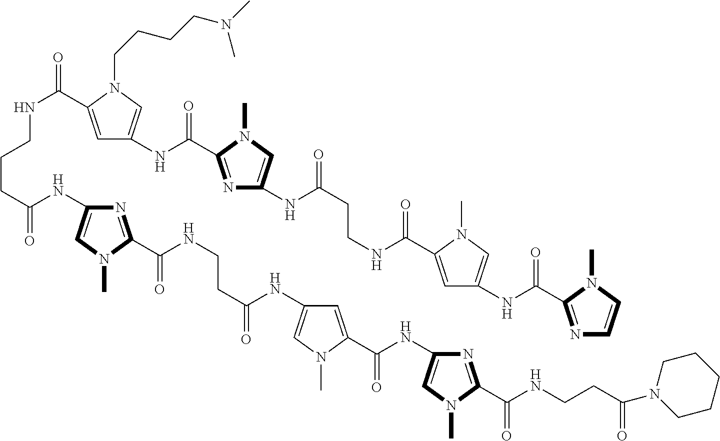 | B-428 |
| 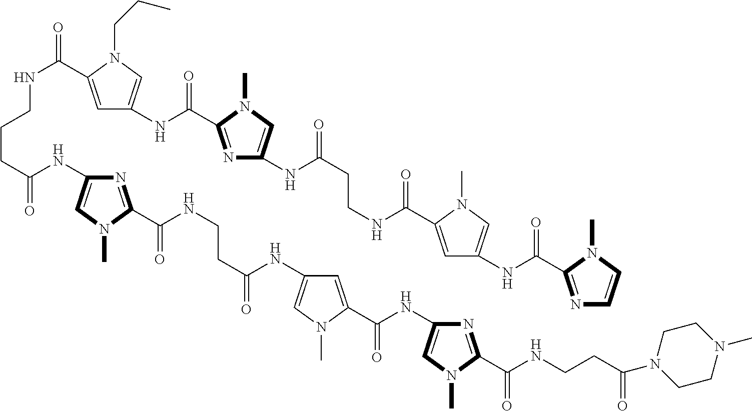 | B-429 |
| 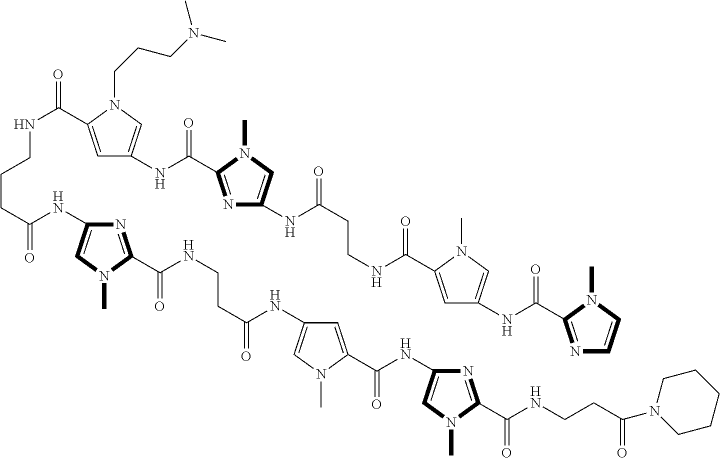 | B-430 |
| 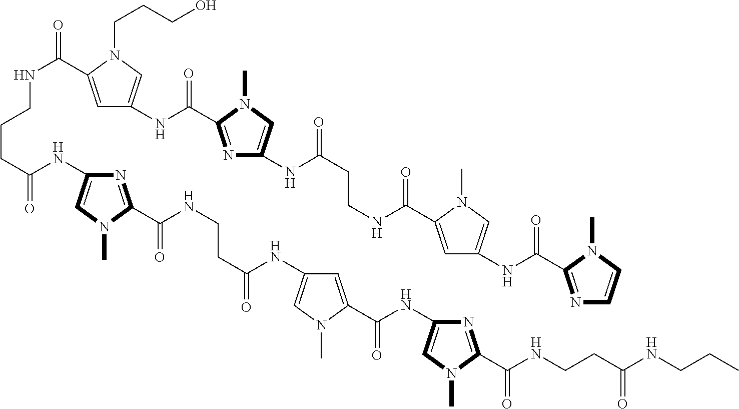 | B-431 |
| 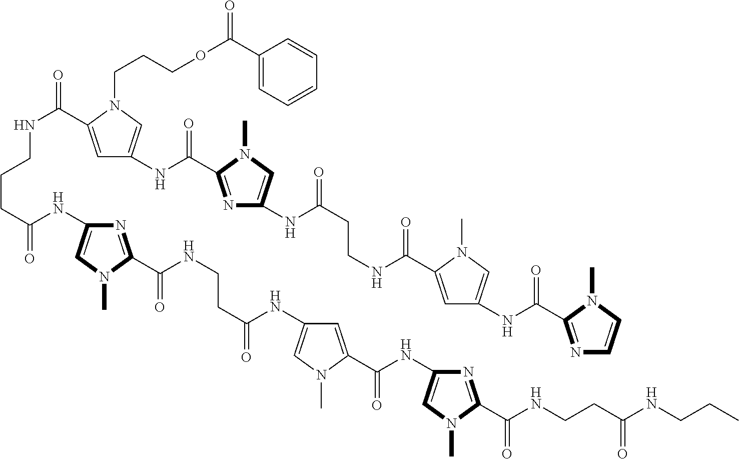 | B-432 |
| 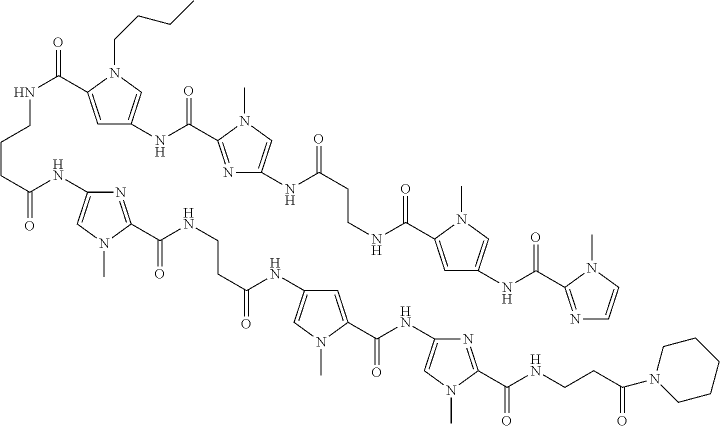 [SMILES: CCCCn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)N4CCCCC4)n3)n(C)c2)n1] | B-433 |
| 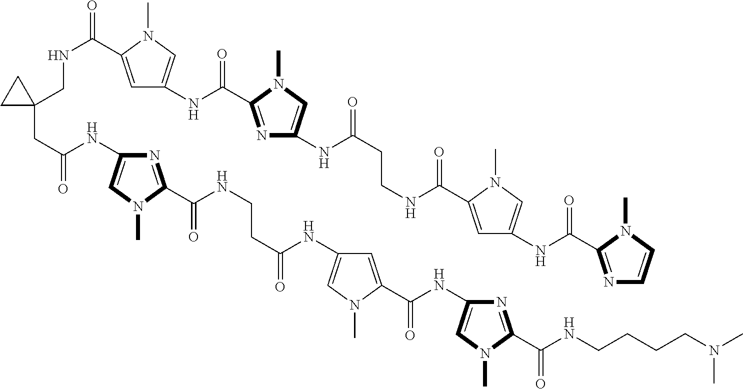 | B-434 |
| 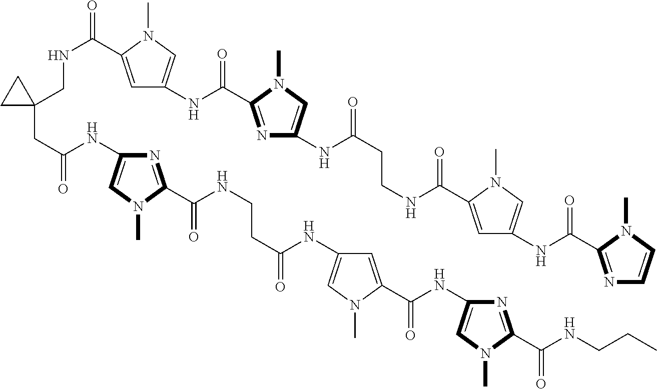 | B-435 |
| 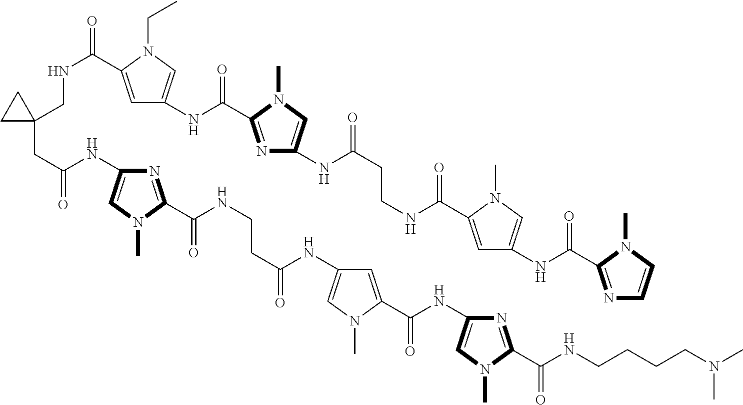 | B-436 |
| 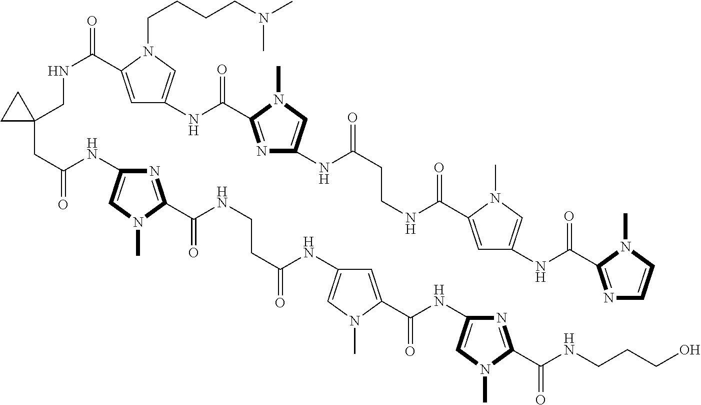 | B-437 |
| 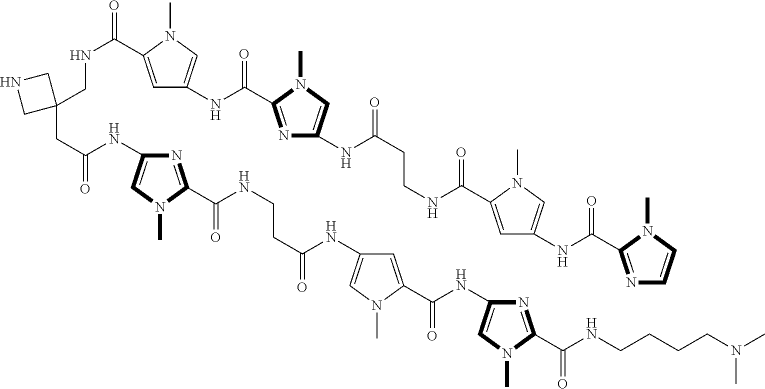 | B-438 |
| 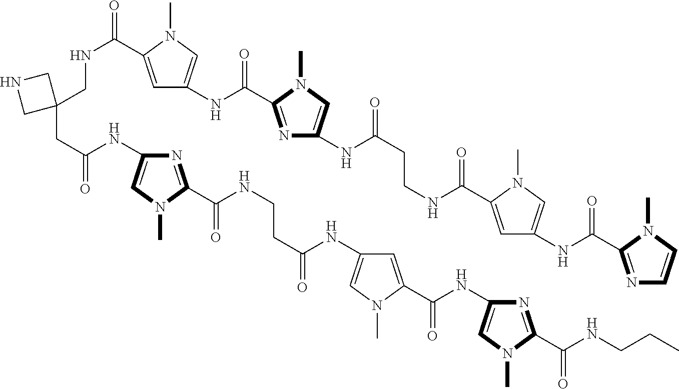 | B-439 |
| 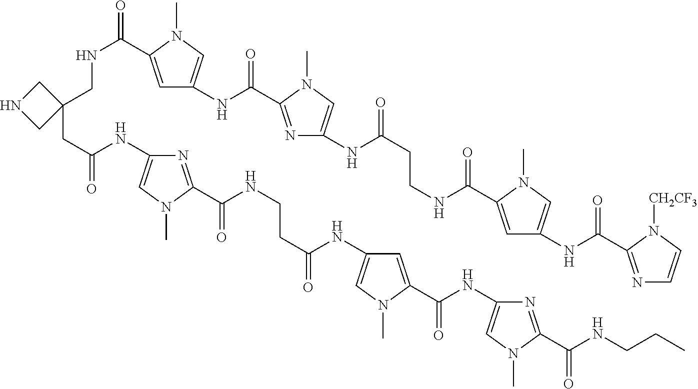 | B-440 |
| 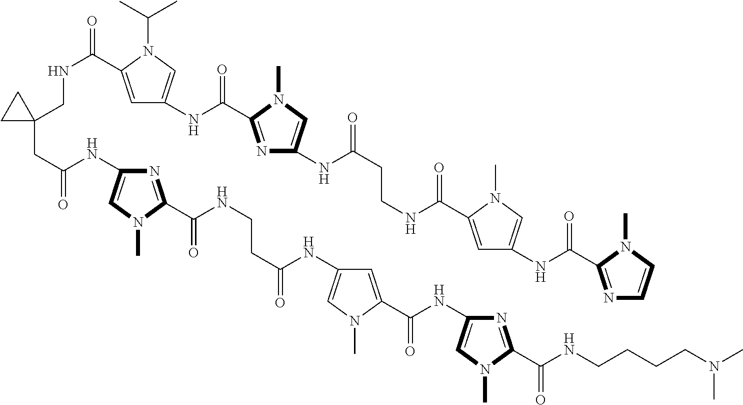 | B-441 |

### Methods of Use

The present disclosure also relates to a method of modulating the transcription of atxn1, atxn2, atxn3, cacna1a, atxn7, ttbk2, ppp2r2b, tbp, htt, jph3, ar, or atn1, the method comprising the step of contacting atxn1, atxn2, atxn3, cacna1a, atxn7, ttbk2, ppp2r2b, tbp, htt, jph3, ar, or atn1 with a transcription modulator molecule as described herein, or a pharmaceutically acceptable salt thereof.

The cell phenotype, cell proliferation, transcription of atxn1, atxn2, atxn3, cacna1a, atxn7, ttbk2, ppp2r2b, tbp, htt, jph3, ar, or atn1; production of mRNA from transcription of atxn1, atxn2, atxn3, cacna1a, atxn7, ttbk2, ppp2r2b, tbp, htt, jph3, ar, or atn1; translation of atxn1, atxn2, atxn3, cacna1a, atxn7, ppp2r2b, tbp, htt, jph3, ar, or atn1; change in biochemical output produced by the protein coded by atxn1, atxn2, atxn3, cacna1a, atxn7, tthk2, ppp2r2b, tbp, htt, jph3, ar, or atn1; or noncovalent binding of the protein coded by atxn1, atxn2, atxn3, cacna1a, atxn7, ttbk2, ppp2r2b, tbp, htt, jph3, ar, or atn1 with a natural binding partner may be monitored. Such methods may be modes of treatment of disease, biological assays, cellular assays, biochemical assays, or the like.

In some embodiments, the gene is atxn2. In some embodiments, the gene is atxn3. In some embodiments, the gene is cacna1a. In some embodiments, the gene is atxn7. In some embodiments, the gene is ppp2r2. In some embodiments, the gene is tbp. In some embodiments, the gene is htt. In some embodiments, the gene is jph3. In some embodiments, the gene is ar. In some embodiments, the gene is atn1. In some embodiments, the gene is ttbk2. In some embodiments, the gene is htt.

Also provided herein is a method of treatment of a disease mediated by transcription of atxn1, atxn2, atxn3, cacna1a, atxn7, ttbk2, ppp2r2b, tbp, htt, jph3, ar, or atn1 comprising the administration of a therapeutically effective amount of a transcription modulator molecule as disclosed herein, or a salt thereof, to a subject in need thereof.

In some embodiments, the disease is selected from spinocerebellar ataxia, Huntington's disease, a Huntington's disease-like syndrome, spinobulbar muscular atrophy, and dentatorubral-pallidoluysian atrophy.

In some embodiments, the disease is spinocerebellar ataxia. In some embodiments, the spinocerebellar ataxia is selected from SCA1, SCA2, SCA3, SCA6, SCA7, SCA12, and SCA17. In some embodiments, the spinocerebellar ataxia is selected from SCA1, SCA2, SCA3, SCA6, SCA7, and SCA17.

In some embodiments, the disease is selected from Huntington's disease and a Huntington's disease-like syndrome. In some embodiments, the disease is selected from Huntington's disease. In some embodiments the disease is selected from Huntington's disease-like 2 syndrome.

In some embodiments, the disease is spinobulbar muscular atrophy.

In some embodiments, the disease is dentatorubral-pallildoluysian atrophy.

### Pharmaceutical Compositions and Administration

The compounds described herein are administered to a subject in need thereof, either alone or in combination with pharmaceutically acceptable carriers, excipients, or diluents, in a pharmaceutical composition, according to standard pharmaceutical practice. In some embodiments, the compounds described herein are administered to animals.

In another aspect, provided herein are pharmaceutical compositions comprising a compound described herein, or a pharmaceutically acceptable salt thereof, and at least one pharmaceutically acceptable excipient. Pharmaceutical compositions are formulated in a conventional manner using one or more pharmaceutically acceptable excipients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, (N.Y., 1980; and Pharmaceutical Dosage Forms and Drug Delivery Systems, Seventh Ed. (Lippincott Williams & Wilkins1999), herein incorporated by reference for such disclosure.

In some embodiments, the pharmaceutically acceptable excipient is selected from carriers, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, and any combinations thereof.

The dose of a pharmaceutical agent described herein for treating a disease or disorder may depend upon the subject's condition, that is, stage of the disease, severity of symptoms caused by the disease, general health status, as well as age, gender, and weight, and other factors apparent to a person skilled in the medical art. Pharmaceutical compositions may be administered in a manner appropriate to the disease to be treated as determined by persons skilled in the medical arts. In addition to the factors described herein and above related to use of pharmaceutical agent for treating a disease or disorder, suitable duration and frequency of administration of the pharmaceutical agent may also be determined or adjusted by such factors as the condition of the patient, the type and severity of the patient's disease, the particular form of the active ingredient, and the method of administration. Optimal doses of an agent may generally be determined using experimental models and/or clinical trials. The optimal dose may depend upon the body mass, weight, or blood volume of the subject. The use of the minimum dose that is sufficient to provide effective therapy is usually preferred. Design and execution of pre-clinical and clinical studies for a pharmaceutical agent, including when administered for prophylactic benefit, described herein are well within the skill of a person skilled in the relevant art. When two or more pharmaceutical agents are administered to treat a disease or disorder, the optimal dose of each pharmaceutical agent may be different, such as less than when either agent is administered alone as a single agent therapy. In certain particular embodiments, two pharmaceutical agents in combination may act synergistically or additively, and either agent may be used in a lesser amount than if administered alone. An amount of a pharmaceutical agent that may be administered per day may be, for example, between about 0.01 mg/kg and 100 mg/kg, e.g., between about 0.1 to 1 mg/kg, between about 1 to 10 mg/kg, between about 10-50 mg/kg, between about 50-100 mg/kg body weight. In other embodiments, the amount of a pharmaceutical agent that may be administered per day is between about 0.01 mg/kg and 1000 mg/kg, between about 100-500 mg/kg, or between about 500-1000 mg/kg body weight. The optimal dose, per day or per course of treatment, may be different for the disease or disorder to be treated and may also vary with the administrative route and therapeutic regimen.

### Abbreviations and Definitions

Unless defined otherwise, all technical and scientific terms used herein have the same meaning as is commonly understood by one of skill in the art to which this invention belongs.

Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two. three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM. 2.9999 μM, etc.).

The terms below, as used herein, have the following meanings, unless indicated otherwise:

“oxo” refers to ═O.

“Carboxyl” refers to —COOH.

“Cyano” refers to —CN.

“Alkyl” refers to a straight-chain, or branched-chain saturated hydrocarbon monoradical having from one to about ten carbon atoms, more preferably one to six carbon atoms. Examples include, but are not limited to methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl and the like. Whenever it appears herein, a numerical range such as “C₁-C₆ alkyl” or “C₁₋₆alkyl”, means that the alkyl group may consist of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁₋₁₀alkyl. In some embodiments, the alkyl is a C₁₋₆alkyl. In some embodiments, the alkyl is a C₁₋₅alkyl. In some embodiments, the alkyl is a C₁₋₄alkyl. In some embodiments, the alkyl is a C₁₋₃alkyl. Unless stated otherwise specifically in the specification, an alkyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkyl is optionally substituted with oxo, halogen, —N₃, —CN, —C(O)OH, —C(O)OMe, —OH, —OMe, —NH₂, or —NO₂. In some embodiments, the alkyl is optionally substituted with halogen, —CN, —OH, or —OMe. In some embodiments, the alkyl is optionally substituted with halogen.

“Alkenyl” refers to a straight-chain, or branched-chain hydrocarbon monoradical having one or more carbon-carbon double-bonds and having from two to about ten carbon atoms, more preferably two to about six carbon atoms. The group may be in either the cis or trans conformation about the double bond(s), and should be understood to include both isomers. Examples include, but are not limited to ethenyl (—CH═CH₂), 1-propenyl (—CH₂CH═CH₂), isopropenyl [—C(CH₃)═CH₂], butenyl, 1,3-butadienyl and the like. Whenever it appears herein, a numerical range such as “C₂-C₆ alkenyl” or “C₂₋₆alkenyl”, means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. Unless stated otherwise specifically in the specification, an alkenyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkenyl is optionally substituted with oxo, halogen, —N₃, —CN, —C(O)OH, —C(O)OMe, —OH, —OMe, —NH₂, or —NO₂. In some embodiments, the alkenyl is optionally substituted with halogen, —CN, —OH, or —OMe. In some embodiments, the alkenyl is optionally substituted with halogen.

“Alkynyl” refers to a straight-chain or branched-chain hydrocarbon monoradical having one or more carbon-carbon triple-bonds and having from two to about ten carbon atoms, more preferably from two to about six carbon atoms. Examples include, but are not limited to ethynyl, 2-propynyl, 2-butynyl, 1,3-butadiynyl and the like. Whenever it appears herein, a numerical range such as “C₂-C₆ alkynyl” or “C₂-C₆alkynyl”, means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated. Unless stated otherwise specifically in the specification, an alkynyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkynyl is optionally substituted with oxo, halogen, —N₃, —CN, —C(O)OH, C(O)OMe, —OH, —OMe, —NH₂, or —NO₂. In some embodiments, the alkynyl is optionally substituted with halogen, —CN, —OH, or —OMe. In some embodiments, the alkynyl is optionally substituted with halogen.

“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkylene is optionally substituted with oxo, halogen, —N₃, —CN, —C(O)OH, C(O)OMe, —OH, —OMe, —NH₂, or —NO₂. In some embodiments, the alkylene is optionally substituted with halogen, —CN, —OH, or —OMe. In some embodiments, the alkylene is optionally substituted with halogen.

“Alkoxy” refers to a radical of the formula —ORₐ where Rₐ is an alkyl radical as defined. Unless stated otherwise specifically in the specification, an alkoxy group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkoxy is optionally substituted with halogen, —N₃, —CN, —C(O)OH, C(O)OMe, —OH, —OMe, —NH₂, or —NO₂. In some embodiments, the alkoxy is optionally substituted with halogen, —CN, —OH, or —OMe. In some embodiments, the alkoxy is optionally substituted with halogen.

“Aryl” refers to a radical derived from an aromatic monocyclic or aromatic multicyclic hydrocarbon ring system by removing a hydrogen atom from a ring carbon atom. The aromatic monocyclic or aromatic multicyclic hydrocarbon ring system can contain only hydrogen and carbon and from five to eighteen carbon atoms, where at least one of the rings in the ring system is aromatic, i.e., it contains a cyclic, delocalized (4n+2) π-electron system in accordance with the Hickel theory. The ring system from which aryl groups are derived include, but are not limited to, groups such as benzene, fluorene, indane, indene, tetralin and naphthalene. The aryl radical may be a monocyclic, bicyclic, tricyclic, or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl (phenyl). Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the aryl is optionally substituted with halogen, methyl, ethyl, —N₃, —CN, —C(O)OH, C(O)OMe, —CF₃, —OH, —OMe, —NH₂, or —NO₂. In some embodiments, the aryl is optionally substituted with halogen, methyl, ethyl, —CN, —CF₃, —OH, or —OMe. In some embodiments, the aryl is optionally substituted with halogen.

“Cycloalkyl” refers to a partially or fully saturated, monocyclic, or polycyclic carbocyclic ring, which may include fused (when fused with an aryl or a heteroaryl ring, the cycloalkyl is bonded through a non-aromatic ring atom), spiro, or bridged ring systems. In some embodiments, the cycloalkyl is fully saturated. Representative cycloalkyls include, but are not limited to, cycloalkyls having from three to fifteen carbon atoms (e.g., C₃-C₁₅ fully saturated cycloalkyl or C₃-C₁₅ cycloalkenyl), from three to ten carbon atoms (e.g., C₃-C₁₀ fully saturated cycloalkyl or C₃-C₁₀ cycloalkenyl), from three to eight carbon atoms (e.g., C₃-C₈ fully saturated cycloalkyl or C₃-C₈ cycloalkenyl), from three to six carbon atoms (e.g., C₃-C₆ fully saturated cycloalkyl or C₃-C₆ cycloalkenyl), from three to five carbon atoms (e.g., C₃-C₅ fully saturated cycloalkyl or C₃-C₅ cycloalkenyl), or three to four carbon atoms (e.g., C₃-C₄ fully saturated cycloalkyl or C₃-C₄ cycloalkenyl). In some embodiments, the cycloalkyl is a 3- to 10-membered fully saturated cycloalkyl or a 3- to 10-membered cycloalkenyl. In some embodiments, the cycloalkyl is a 3- to 6-membered fully saturated cycloalkyl or a 3- to 6-membered cycloalkenyl. In some embodiments, the cycloalkyl is a 5- to 6-membered fully saturated cycloalkyl or a 5- to 6-membered cycloalkenyl. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example cyclopentenyl, cyclohexenyl, cycloheptenyl. and cyclooctenyl. Unless stated otherwise specifically in the specification, a cycloalkyl is optionally substituted, for example, with oxo. halogen, amino, nitrile, nitro, hydroxyl. alkyl, alkenyl, alkynyl, haloalkyl, alkoxy, carboxyl, carboxylate, aryl, cycloalkyl, heterocycloalkyl, heteroaryl, and the like. In some embodiments, a cycloalkyl is optionally substituted with oxo, halogen, methyl, ethyl, —N₃, —CN, —C(O)OH, C(O)OMe, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Cycloalkenyl” refers to an unsaturated non-aromatic monocyclic or polycyclic hydrocarbon radical consisting solely of carbon and hydrogen atoms, which includes fused or bridged ring systems, preferably having from three to twelve carbon atoms and comprising at least one double bond. In certain embodiments, a cycloalkenyl comprises three to ten carbon atoms. In other embodiments, a cycloalkenyl comprises five to seven carbon atoms. The cycloalkenyl may be attached to the rest of the molecule by a single bond. Examples of monocyclic cycloalkenyls includes, e.g., cyclopentenyl, cyclohexenyl, cycloheptenyl, and cyclooctenyl.

“Halo” or “halogen” refers to bromo, chloro, fluoro or iodo. In some embodiments, halogen is fluoro or chloro. In some embodiments, halogen is fluoro.

As used herein, the term “haloalkyl” or “haloalkane” refers to an alkyl radical, as defined above, that is substituted by one or more halogen radicals, for example, trifluoromethyl, dichloromethyl, bromomethyl, 2,2,2-trifluoroethyl, 1-fluoromethyl-2-fluoroethyl, and the like. In some embodiments, the alkyl part of the fluoroalkyl radical is optionally further substituted. Examples of halogen substituted alkanes (“haloalkanes”) include halomethane (e.g., chloromethane, bromomethane, fluoromethane, iodomethane), di- and trihalomethane (e.g., trichloromethane, tribromomethane, trifluoromethane, triiodomethane), 1-haloethane, 2-haloethane, 1,2-dihaloethane, 1-halopropane, 2-halopropane, 3-halopropane, 1,2-dihalopropane, 1,3-dihalopropane, 2,3-dihalopropane. 1,2,3-trihalopropane, and any other suitable combinations of alkanes (or substituted alkanes) and halogens (e.g., Cl, Br, F, I, etc.). When an alkyl group is substituted with more than one halogen radicals, each halogen may be independently selected e.g., 1-chloro,2-fluoroethane.

“Fluoroalkyl” refers to an alkyl radical, as defined above, that is substituted by one or more fluoro radicals, for example, trifluoromethyl, difluoromethyl, fluoromethyl, 2,2,2-trifluoroethyl, 1-fluoromethyl-2-fluoroethyl, and the like.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

“Aminoalkyl” refers to an alkyl radical, as defined above, that is substituted by one or more amines. In some embodiments, the alkyl is substituted with one amine. In some embodiments, the alkyl is substituted with one, two, or three amines. Aminoalkyl include, for example, aminomethyl, aminoethyl, aminopropyl, aminobutyl, or aminopentyl. In some embodiments, the aminoalkyl is aminomethyl.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, phosphorus, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, phosphorus, or combinations thereof wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, —CH(CH₃)OCH₃, —CH₂NHCH₃, —CH₂N(CH₃)₂, —CH₂CH₂NHCH₃, or —CH₂CH₂N(CH₃)₂. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Heterocycloalkyl” refers to a 3- to 24-membered partially or fully saturated ring radical comprising 2 to 23 carbon atoms and from one to 8 heteroatoms selected from the group consisting of nitrogen, oxygen, phosphorous, silicon, and sulfur. In some embodiments, the heterocycloalkyl is fully saturated. In some embodiments, the heterocycloalkyl comprises one to three heteroatoms selected from the group consisting of nitrogen, oxygen, and sulfur. In some embodiments, the heterocycloalkyl comprises one to three heteroatoms selected from the group consisting of nitrogen and oxygen. In some embodiments, the heterocycloalkyl comprises one to three nitrogens. In some embodiments, the heterocycloalkyl comprises one or two nitrogens. In some embodiments, the heterocycloalkyl comprises one nitrogen. In some embodiments, the heterocycloalkyl comprises one nitrogen and one oxygen. Unless stated otherwise specifically in the specification, the heterocycloalkyl radical may be a monocyclic, bicyclic, tricyclic, or tetracyclic ring system, which may include fused (when fused with an aryl or a heteroaryl ring, the heterocycloalkyl is bonded through a non-aromatic ring atom), spiro, or bridged ring systems; and the nitrogen, carbon, or sulfur atoms in the heterocycloalkyl radical may be optionally oxidized; the nitrogen atom may be optionally quaternized. Representative heterocycloalkyls include, but are not limited to, heterocycloalkyls having from two to fifteen carbon atoms (e.g., C₂-C₁₅ fully saturated heterocycloalkyl or C₂-C₁₅ heterocycloalkenyl), from two to ten carbon atoms (e.g., C₂-C₁₀ fully saturated heterocycloalkyl or C₂-C₁₀ heterocycloalkenyl), from two to eight carbon atoms (e.g., C₂-C₈ fully saturated heterocycloalkyl or C₂-C₈ heterocycloalkenyl), from two to seven carbon atoms (e.g., C₂-C₇ fully saturated heterocycloalkyl or C₂-C₇ heterocycloalkenyl), from two to six carbon atoms (e.g., C₂-C₆ fully saturated heterocycloalkyl or C₂-C₆ heterocycloalkenyl), from two to five carbon atoms (e.g., C₂-C₅ fully saturated heterocycloalkyl or C₂-C₅ heterocycloalkenyl), or two to four carbon atoms (e.g., C₂-C₄ fully saturated heterocycloalkyl or C₂-C₄ heterocycloalkenyl). Examples of such heterocycloalkyl radicals include, but are not limited to, aziridinyl, azetidinyl, oxetanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to the monosaccharides, the disaccharides, and the oligosaccharides. In some embodiments, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). In some embodiments, the heterocycloalkyl is a 3- to 8-membered fully saturated heterocycloalkyl. In some embodiments, the heterocycloalkyl is a 3- to 7-membered fully saturated heterocycloalkyl. In some embodiments, the heterocycloalkyl is a 3- to 6-membered fully saturated heterocycloalkyl. In some embodiments, the heterocycloalkyl is a 4- to 6-membered fully saturated heterocycloalkyl. In some embodiments, the heterocycloalkyl is a 5- to 6-membered fully saturated heterocycloalkyl. In some embodiments, the heterocycloalkyl is a 3- to 8-membered heterocycloalkenyl. In some embodiments, the heterocycloalkyl is a 3- to 7-membered heterocycloalkenyl. In some embodiments, the heterocycloalkyl is a 3- to 6-membered heterocycloalkenyl. In some embodiments, the heterocycloalkyl is a 4- to 6-membered heterocycloalkenyl. In some embodiments, the heterocycloalkyl is a 5- to 6-membered heterocycloalkenyl. Unless stated otherwise specifically in the specification, a heterocycloalkyl may be optionally substituted as described below, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heterocycloalkyl is optionally substituted with oxo, halogen, methyl, ethyl, —CN, —C(O)OH, C(O)OMe, —CF₃, —OH, —OMe, —NH₂, or —NO₂. In some embodiments, the heterocycloalkyl is optionally substituted with halogen, methyl, ethyl, —CN, —CF₃, —OH, or —OMe. In some embodiments, the heterocycloalkyl is optionally substituted with halogen.

“Heteroaryl” refers to a 5- to 14-membered ring system radical comprising one to thirteen carbon atoms, one to six heteroatoms selected from the group consisting of nitrogen, oxygen, phosphorous, and sulfur, and at least one aromatic ring. In some embodiments, the heteroaryl comprises one to three heteroatoms selected from the group consisting of nitrogen, oxygen, and sulfur. In some embodiments, the heteroaryl comprises one to three heteroatoms selected from the group consisting of nitrogen and oxygen. In some embodiments, the heteroaryl comprises one to three nitrogens. In some embodiments, the heteroaryl comprises one or two nitrogens. In some embodiments, the heteroaryl comprises one nitrogen. The heteroaryl radical may be a monocyclic, bicyclic, tricyclic, or tetracyclic ring system, which may include fused (when fused with a cycloalkyl or heterocycloalkyl ring, the heteroaryl is bonded through an aromatic ring atom) or bridged ring systems; and the nitrogen, carbon, or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. In some embodiments, the heteroaryl is a 6-membered heteroaryl. In some embodiments, the heteroaryl is a 5-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl may be optionally substituted, for example, with halogen, amino, nitrile, nitro. hydroxyl, alkyl, alkenyl, alkynyl. haloalkyl. alkoxy, carboxyl, carboxylate, aryl, cycloalkyl, heterocycloalkyl, heteroaryl, and the like. In some embodiments, the heteroaryl is optionally substituted with halogen, methyl, ethyl, —CN, —C(O)OH, C(O)OMe, —CF₃, —OH, —OMe, —NH₂, or —NO₂. In some embodiments, the heteroaryl is optionally substituted with halogen, methyl, ethyl, —CN, —CF₃, —OH, or —OMe. In some embodiments, the heteroaryl is optionally substituted with halogen.

The term “oligonucleotide sequence” refers to a plurality of nucleic acids having a defined sequence and length (e.g., 2, 3, 4, 5, 6, or even more nucleotides). The term “oligonucleotide repeat sequence” refers to a contiguous expansion of oligonucleotide sequences.

The term “transcription,” well known in the art, refers to the synthesis of RNA (i.e., ribonucleic acid) by DNA-directed RNA polymerase. The term “modulate transcription” refers to a change in transcriptional level which can be measured by methods well known in the art, for example, assay of mRNA, the product of transcription. In certain embodiments, modulation is an increase in transcription. In other embodiments, modulation is a decrease in transcription.

The term “polyamide” refers to polymers of linkable units chemically bound by amide (i.e., CONH) linkages; optionally, polyamides include chemical probes conjugated therewith. Polyamides may be synthesized by stepwise condensation of carboxylic acids (COOH) with amines (RR′NH) using methods known in the art. Alternatively, polyamides may be formed using enzymatic reactions in vitro, or by employing fermentation with microorganisms.

The term “linkable unit” refers to methylimidazoles, methylpyrroles, and straight and branched chain aliphatic functionalities (e.g., methylene, ethylene, propylene, butylene, and the like) which optionally contain nitrogen Substituents, and chemical derivatives thereof. The aliphatic functionalities of linkable units can be provided, for example, by condensation of B-alanine or dimethylaminopropylamine during synthesis of the polyamide by methods well known in the art.

The term “linker” or “oligomeric backbone” refers to a chain of at least 10 contiguous atoms. In certain embodiments, the linker contains no more than 20 non-hydrogen atoms. The terms linker and oligomeric backbone can be used interchangeably. In some embodiments, the linker contains no more than 40 non-hydrogen atoms. In some embodiments, the linker contains no more than 60 non-hydrogen atoms. In certain embodiments, the linker contains atoms chosen from C, H, N, O, and S. In some embodiments, every non-hydrogen atom is chemically bonded either to 2 neighboring atoms in the linker, or one neighboring atom in the linker and a terminus of the linker. In some embodiments, the linker forms an amide bond with at least one of the two other groups to which it is attached. In certain embodiments, the linker forms an ester or ether bond with at least one of the two other groups to which it is attached. In some embodiments, the linker forms a thioester or thioether bond with at least one of the two other groups to which it is attached. In some embodiments, the linker forms a direct carbon-carbon bond with at least one of the two other groups to which it is attached. In some embodiments, the linker forms an amine or amide bond with at least one of the two other groups to which it is attached. In some embodiments, the linker comprises —(CH₂OCH₂)— units. In some embodiments, the linker comprises —(CH(CH₃)OCH₂)-units. In some embodiments, the linker comprises —(CH₂NR_{N}CH₂) units, for R_{N}═C₁₋₄alkyl. In some embodiments, the linker comprises an arylene, cycloalkylene, or heterocycloalkylene moiety.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

As used herein, “optionally substituted” is a substituted group is derived from the unsubstituted parent group in which there has been an exchange of one or more hydrogen atoms for another atom or group. Unless otherwise indicated, when a group is deemed to be “substituted” or “optionally substituted” it is meant that the group is substituted with one or more substituents independently selected from C₁-C₆ alkyl, C₁-C₆ alkenyl, C₁-C₆ alkynyl, C₁-C₆ heteroalkyl. C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 3-10 membered heterocyclyl (optionally substituted with halo, C₁-C₆ alkyl, C₁-C₆ alkoxy, C₁-C₆ haloalkyl, and C₁-C₆ haloalkoxy), 3-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), aryl(C₁-C₆)alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkyl (optionally substituted with halo, C₁-C₆ alkyl, C₁-C₆ alkoxy, C₁-C₆ haloalkyl, and C₁-C₆ haloalkoxy), halo, cyano, hydroxy, C₁-C₆ alkoxy, C₁-C₆ alkoxy(C₁-C₆)alkyl (i.e., ether), aryloxy, sulfhydryl (mercapto), halo(C₁-C₆)alkyl (e.g., —CF₃), halo(C₁-C₆)alkoxy (e.g., —OCF₃), C₁-C₆ alkylthio, arylthio, amino, amino(C₁-C₆)alkyl, nitro, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, and oxo (═O). Wherever a group is described as “optionally substituted” that group can be substituted with the above substituents.

The term “one or more” when referring to an optional substituent means that the subject group is optionally substituted with one, two, three, or four substituents. In some embodiments, the subject group is optionally substituted with one, two, or three substituents. In some embodiments, the subject group is optionally substituted with one or two substituents. In some embodiments, the subject group is optionally substituted with one substituent. In some embodiments, the subject group is optionally substituted with two substituents.

Chemical entities having carbon-carbon double bonds or carbon-nitrogen double bonds may exist in Z- or E-form (or cis- or trans-form). Furthermore, some chemical entities may exist in various tautomeric forms. Unless otherwise specified, compounds described herein are intended to include all Z—, E- and tautomeric forms as well.

The compounds disclosed herein, in some embodiments, are used in different enriched isotopic forms, e.g., enriched in the content of ²H, ³H, ¹¹C, ³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, compounds described herein are intended to include compounds which differ only in the presence of one or more isotopically enriched atoms. For example. compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, and ¹²⁵I are all contemplated. All isotopic variations of the compounds of the present invention, whether radioactive or not, are encompassed within the scope of the present invention. In some embodiments, where isotopic variations are illustrated, the remaining atoms of the compound may optionally contain unnatural portions of atomic isotopes.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)]2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

In some embodiments of a compound disclosed herein, one or more of the substituent groups comprise deuterium at a percentage higher than the natural abundance of deuterium. In some embodiments of a compound disclosed herein, one or more hydrogens are replaced with one or more deuteriums.

In some embodiments of a compound disclosed herein, the abundance of deuterium in each of the substituents is independently at least 1%, at least 10%, at least 20%, at least 30%, at least 40%, at least 50%, at least 60%, at least 70%, at least 80%, at least 90%, or 100% of a total number of hydrogen and deuterium.

Compounds of the present disclosure also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

The compounds described herein may in some cases exist as diastereomers, enantiomers, or other stereoisomeric forms. Where absolute stereochemistry is not specified, the compounds presented herein include all diastereomeric, enantiomeric, and epimeric forms as well as the appropriate mixtures thereof. Separation of stereoisomers may be performed by chromatography or by forming diastereomers and separating by recrystallization, or chromatography, or any combination thereof. (Jean Jacques, Andre Collet, Samuel H. Wilen, “Enantiomers, Racemates and Resolutions”, John Wiley And Sons, Inc., 1981, herein incorporated by reference for this disclosure). Stereoisomers may also be obtained by stereoselective synthesis.

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine. trimethylamine, diethylamine, triethylamine, tripropylamine, and ethanolamine. In some embodiments, the pharmaceutically acceptable base addition salt is chosen from ammonium, potassium, sodium, calcium, and magnesium salts.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity. irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition, or vehicle, such as a liquid or solid filler, diluent, excipient, solvent, or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols. such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

An “effective amount” or “therapeutically effective amount” refers to an amount of a compound administered to a mammalian subject, either as a single dose or as part of a series of doses, which is effective to produce a desired therapeutic effect.

The terms “treat,” “treating” or “treatment,” as used herein, include alleviating, abating, or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition.

The term “patient” is generally synonymous with the term “subject” and includes all mammals including humans. Examples of patients include humans, livestock such as cows, goats, sheep, pigs, and rabbits, and companion animals such as dogs, cats, rabbits, and horses. Preferably, the patient is a human.

The term “contacting” refers to bringing the compound (e.g. a transcription molecular molecule of the present disclosure) into proximity of the desired target gene. The contacting may result in the binding to or result in a conformational change of the target moiety.

The methods and compositions described herein include the use of amorphous forms as well as crystalline forms (also known as polymorphs). The compounds described herein may be in the form of pharmaceutically acceptable salts. As well, in some embodiments, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Compound Synthesis

Compounds of the present disclosure can be prepared using methods illustrated in general synthetic schemes and experimental procedures detailed below. General synthetic schemes and experimental procedures are presented for purposes of illustration and are not intended to be limiting. Starting materials used to prepare compounds of the present disclosure are commercially available or can be prepared using routine methods known in the art.

Synthetic chemistry transformations and methodologies useful in synthesizing the compounds described herein are known in the art and include, for example, those described in R. Larock, Comprehensive Organic Transformations (1989); T. W. Greene and P. G. M. Wuts, Protective Groups in Organic Synthesis, 2d. Ed. (1991); L. Fieser and M. Fieser, Fieser and Fieser's Reagents for Organic Synthesis (1994); and L. Paquette, ed., Encyclopedia of Reagents for Organic Synthesis (1995).

### List of Abbreviation

Ac₂O=acetic anhydride; AcCl=acetyl chloride; ACN═acetonitrile: AcOH=acetic acid; AIBN═azobisisobutyronitrile; aq.=aqueous; Bu₃SnH=tributyltin hydride; CD₃OD=deuterated methanol; CDCl₃=deuterated chloroform; CDI=1,1′-Carbonyldiimidazole; DBU=1,8-diazabicyclo[5.4.0]undec-7-ene; DCM=dichloromethane; DEAD=diethyl azodicarboxylate; DIBAL-H=di-iso-butyl aluminium hydride; DIEA=DIPEA=N,N-diisopropylethylamine; DMAP=4-dimethylaminopyridine; DMF=N,N-dimethylformamide; DMSO-d₆=deuterated dimethyl sulfoxide; DMSO=dimethyl sulfoxide; DPPA=diphenylphosphoryl azide; EDC·HCl=EDCl·HCl=1-ethyl-3-(3-dimethylaminopropyl)carbodiimide hydrochloride; Et₂O=diethyl ether; EA=ethyl acetate; EtOH=ethanol; h=hour; HATU=2-(1H-7-azabenzotriazol-1-yl)-1,1,3,3-tetramethyl uronium hexafluorophosphate methanaminium; HMDS=hexamethyldisilazane; HOBT=1-hydroxybenzotriazole; i-PrOH=isopropanol; LAH=lithium aluminium hydride; LiHMDS=Lithium bis(trimethylsilyl)amide; MeCN═acetonitrile; MeOH=methanol; MP-carbonate resin=macroporous triethylammonium methylpolystyrene carbonate resin; MsCl=mesyl chloride; MTBE=methyl tertiary butyl ether; MW=microwave irradiation; n-BuLi=n-butyllithium; NaHMDS=Sodium bis(trimethylsilyl)amide; NaOMe=sodium methoxide; NaOtBu=sodium t-butoxide; NBS=N-bromosuccinimide; NCS=N-chloro-succinimide; NMP=N-Methyl-2-pyrrolidone; Pd(Ph₃)₄=tetrakis(triphenylphosphine)palladium(0); Pd₂(dba)₃=tris(dibenzylideneacetone)dipalladium(0); PdCl₂(PPh₃)₂=bis(triphenylphosphine)palladium(II) dichloride; PG=protecting group; prep-HPLC=preparative high-performance liquid chromatography; PyBop=(benzotriazol-1-yloxy)tripyrrolidinophosphonium hexafluorophosphate; Pyr=pyridine; RT=room temperature; RuPhos=2-dicyclohexylphosphino-2′,6′-diisopropoxybiphenyl; sat.=saturated; ss=saturated solution; t-BuOH=tert-butanol; T3P=Propylphosphonic Anhydride; TBS=TBDMS=tert-butyldimethylsilyl; TBSCl=TBDMSCl=tert-butyldimethylchlorosilane; TEA=Et₃N═triethylamine; TFA=trifluoroacetic acid; TFAA=trifluoroacetic anhydride; THF=tetrahydrofuran; Tol=toluene; TsCl=tosyl chloride; XPhos=2-dicyclohexylphosphino-2′,4′,6′-triisopropylbiphenyl.

### Synthesis of Representative Polyamides

### Example 1. Synthesis of 1-methyl-4-(3-(1-methyl-4-(1-methyl-4-(3-(1-methyl-4-(1-methyl-4-(3-(1-methyl-1H-pyrrole-2-carboxamido)propanamido)-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxylic acid (PA-03)

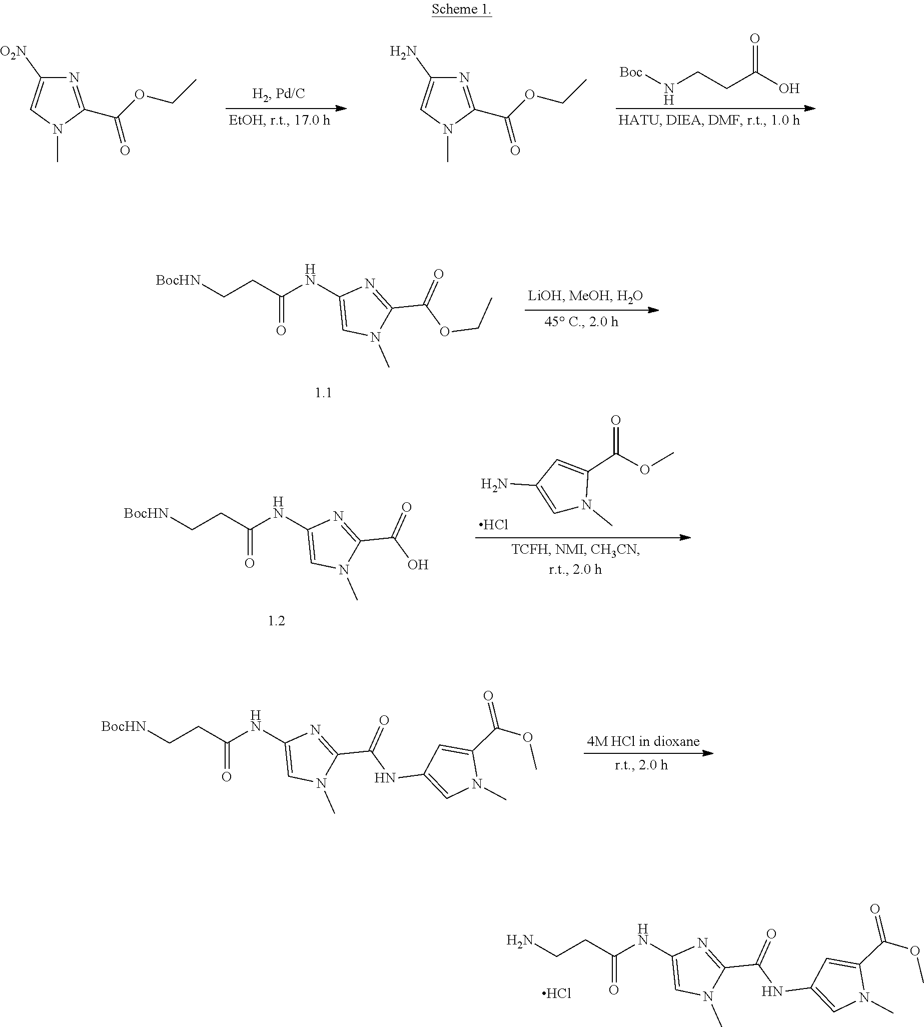

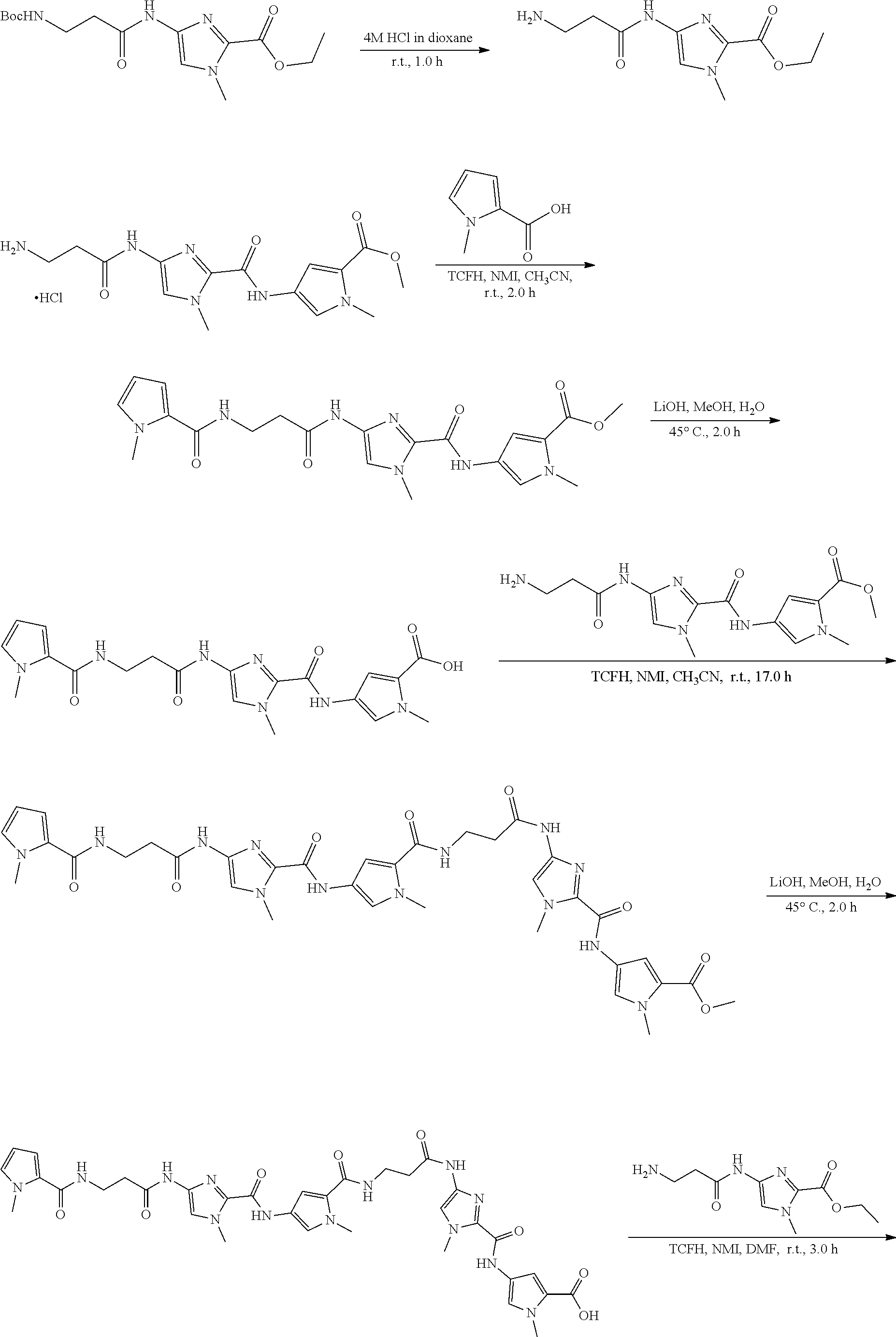

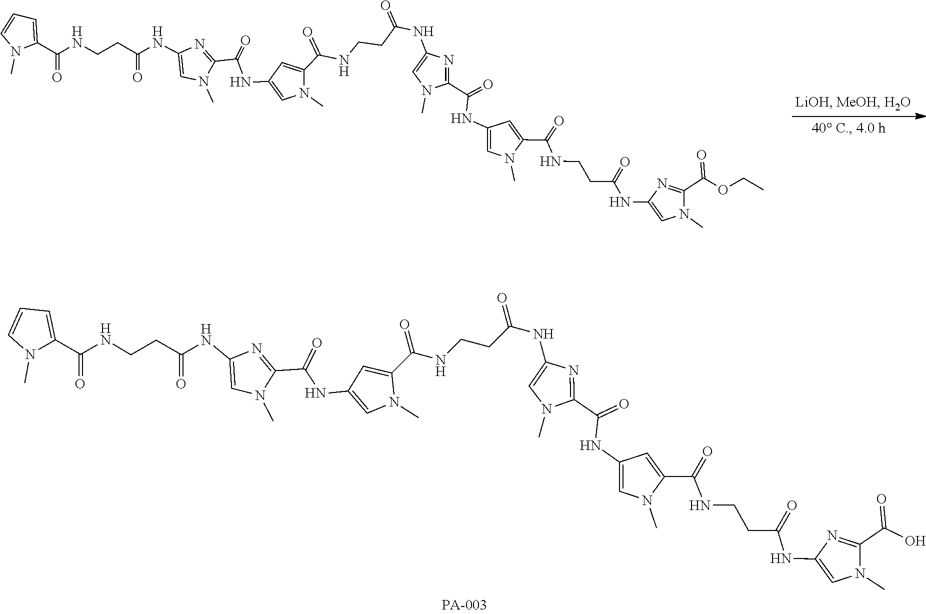 [SMILES: CCOC(=O)c1nc(NC(=O)CCNC(=O)c2cc(NC(=O)c3nc(NC(=O)CCNC(=O)c4cc(NC(=O)c5nc(NC(=O)CCNC(=O)c6cccn6C)cn5C)cn4C)cn3C)cn2C)cn1C ; Cn1cc(NC(=O)c2nc(NC(=O)CCNC(=O)c3cc(NC(=O)c4nc(NC(=O)CCNC(=O)c5cccn5C)cn4C)cn3C)cn2C)cc1C(=O)NCCC(=O)Nc1cn(C)c(C(=O)O)n1]

Step 1: To a solution of ethyl 1-methyl-4-nitroimidazole-2-carboxylate (30.00 g, 150.63 mmol, 1.00 equiv) in EtOH (120.00 mL) and EA (120.00 mL) was added Pd/C (8.01 g, 27% w/w). Then the reaction was stirred for 17.0 h at room temperature under H₂ atmosphere. The solid was filtrated out and the filtrate was concentrated to afford ethyl 4-amino-1-methylimidazole-2-carboxylate (22.30 g, 75.20%) as a yellow solid. LC/MS: mass calcd. For C₇H₁₁N₃O₂: 169.09, found: 170.10 [M+H]⁺.

Step 2: Into a 500 mL flask was added 3-[(tert-butoxycarbonyl) amino]propanoic acid (22.45 g, 118.65 mmol, 0.90 equiv) in DMF (180.00 mL). The mixture was cooled to 0° C., then HATU (75.18 g, 197.71 mmol, 1.50 equiv) and DIEA (51.11 g, 395.43 mmol, 3.00 equiv) were added and the mixture was stirred for 10 mins. Then ethyl 4-amino-1-methylimidazole-2-carboxylate (22.30 g, 131.81 mmol, 1.00 equiv) was added in portions and the reaction was stirred at room temperature for 1.0 h. The reaction was quenched with ice water (600 mL), and the solution was stirred for 15 min. The precipitated solids were collected by filtration and washed with water (3×50 mL) and dried under vacuum. This resulted in ethyl 4-[3-[(tert-butoxycarbonyl)amino]propanamido]-1-methylimidazole-2-carboxylate (34.50 g, 76.90%) as a light yellow solid. LC/MS: mass calcd. For C₁₅H₂₄N₄O₅: 340.17, found: 341.20 [M+H]⁺.

Step 3: To a stirred solution of ethyl 4-|3-|(tert-butoxycarbonyl)amino|propanamido| 1Methylimidazole-2-carboxylate (34.50 g, 101.36 mmol, 1.00 equiv) in MeOH (200.00 mL) was added LiOH solution (2 M, 202 mL, 4.00 equiv) dropwise at room temperature. The resulting mixture was stirred for 2.0 h at 45° C. The mixture the was concentrated under reduced pressure. The residue was dissolved in H₂O (50 mL) and s acidified to pH 3~5 with 2M HCl. The precipitated solids were collected by filtration and washed with H₂O (3×30 mL) and dried under vacuum to afford 4-[3-[(Tert-butoxycarbonyl)amino]propanamido]-1-methylimidazole-2-carboxylic acid (30.00 g, 94.77%) as a white solid. LC/MS: mass calcd. For C₁₃H₂₀N₄O₅: 312.14, found: 313.15 [M+H]⁺.

Step 4: To a stirred solution of 4-[3-[(tert-butoxycarbonyl)amino]propanamido-1-methylimidazole-2-carboxylic acid (16.00 g, 51.23 mmol, 1.00 equiv) in CH₃CN (150.00 mL) was added TCFH (21.56 g, 76.84 mmol, 1.50 equiv), NMI (12.62 g, 153.69 mmol, 3.00 equiv) and methyl 4-amino-1-methylpyrrole-2-carboxylate hydrochloride (10.74 g, 56.34 mmol, 1.10 equiv) in portions at 0° C. The resulting mixture was stirred for 2.0 h at room temperature. The precipitated solids were collected by filtration, washed with CH₃CN (3×20 mL), and dried under vacuum. Methyl 4-(4-[3-[(tert-butoxycarbonyl)amino]propanamido-1-methylimidazole-2-amido)-1-methylpyrrole-2-carboxylate (19.00 g, 82.70%) was obtained as a white solid. LC/MS: mass calcd. For C₂₀H₂₈N₆O₆: 448.21, found: 449.25 [M+H]⁺.

Step 5: A solution of methyl 4-(4-[3-[(tert-butoxycarbonyl)amino]propanamido]-1-methylimidazole-2-amido)-1-methylpyrrole-2-carboxylate (19.00 g, 42.37 mmol, 1.00 equiv) in HCF1,4-dioxane (4M, 200.00 mL) was stirred for 2 h at room temperature. The resulting mixture was concentrated under vacuum to afford methyl 4-[4-(3-aminopropanamido)-1-methylimidazole-2-amido]-1-methylpyrrole-2-carboxylate hydrochloride (19.00 g crude) as a yellow solid. LC/MS: mass calcd. For C₁₅H₂₁ClN₆O₄: 348.15, found: 349.05 [M+H]^{T}.

Step 6: The procedure was the same as methyl 4-[4-(3-aminopropanamido)-1-methylimidazole-2-amido]-1-methylpyrrole-2-carboxylate hydrochloride. But 2.00 g of ethyl 4-[3-[(tert-butoxycarbonyl) amino]propanamido]-1-methylimidazole-2-carboxylate was used, and 2.00 g of crude desired product was obtained as an off-white solid. LC/MS: mass calcd. For C₁₀H₁₆N₄O₃: 240.12, found: 241.10 [M+H]⁺.

Step 7: To a solution of 1-methylpyrrole-2-carboxylic acid (600.00 mg, 4.80 mmol, 1.00 equiv) in CH₃CN (20.00 mL) was added NMI (1.22 g, 14.87 mmol, 3.10 equiv), TCFH (1.48 g, 5.28 mmol, 1.10 equiv) and methyl 4-[4-(3-aminopropanamido)-1-methylimidazole-2-amido]-1-methylpyrrole-2-carboxylate (2004.53 mg, 5.75 mmol, 1.20 equiv). The mixture was stirred at room temperature for 2.0 h. Next, the solvent was removed and the residue was purified by reverse phase column under the condition: Column, C18 column, MeCN/water (0.05% TFA), 5% to 50% gradient in 100 min-; detector, 254 nm. The fractions were combined and concentrated to afford 1.30 g of the desired product as a white solid (56% yield). LC/MS: mass calcd. For C₂₁H₂₅N₇O₅: 455.19, found: 456.30 [M+H]⁺.

Step 8: The procedure was the same as 4-[3-[(tert-butoxycarbonyl)amino]propanamido]-1-methylimidazole-2-carboxylic acid. But 2.00 g of methyl 1-methyl-4-(1-methyl-4-[3-[(1-methylpyrrol-2-yl)formamido propanamido|imidazole-2-amido)pyrrole-2-carboxylate was used to obtain 1.90 g of the desired product as a white solid (92.00% yield). LC/MS: mass calcd. For C₂₀H₂₃N₇O₅: 441.18, found: 442.25 [M+H]⁺.

Step 9: The procedure was the same as methyl 4-(4-[3-[(tert-butoxycarbonyl)amino]propanamido]-1-methylimidazole-2-amido)-1-methylpyrrole-2-carboxylate, but the filtrate was concentrated and purified by reverse phase column. The reaction was run with 1.90 g of 1-methyl-4-(1-methyl-4-[3-[(1-methylpyrrol-2-yl)formamido]propanamido]imidazole-2-amido)pyrrole-2-carboxylic acid to obtain 2.70 g of the desired product as a white solid (71.00% yield). LC/MS: mass calcd. For C₃₅H₄₁N₁₃O: 771.32, found: 772.35 [M+H]⁺.

Step 10: The procedure was the same as 4-[3-[(tert-butoxycarbonyl)amino]propanamido]-1-methylimidazole-2-carboxylic acid but 2.70 g of methyl 1-methyl-4-[1-methyl-4-(3-[[1-methyl-4-(1-methyl-4-[3-[(1-methylpyrrol-2-yl) formamido]propanamido]imidazole-2-amido)pyrrol-2-yl]formamnido]propanamido)imidazole-2-amido]pyrrole-2-carboxylate was used to obtain 2.80 g of the desired product as a white solid (78.00% yield). LC/MS: mass calcd. For C₃₄H₃₉N₁₃O: 757.30, found: 758.50 [M+H]⁺.

Step 11: To a solution of 1-methyl-4-[1-methyl-4-(3-[[1-methyl-4-(1-methyl-4-[3-[(1-methylpyrrol-2-1-methyl-4-[I-methyl-4-(3-[[1-methyl-4-(I-methyl-4-[3-[(1-methylpyrrol-2-yl)formamido propanamido|imidazole-2-amido)pyrrol-2-yl|formamido]propanamido)imidazole-2-amido]pyrrole-2-carboxylic acid (2.90 g, 3.83 mmol, 1.00 equiv) in DMF (25.00 mL) was added NMI (3.20 g, 39.04 mmol, 10.20 equiv), TCFH (1.18 g, 4.21 mmol, 1.10 equiv) and ethyl 4-(3-aminopropanamido)-1-methylimidazole-2-carboxylate (1.16 g, 4.21 mmol, 1.10 equiv). Then reaction was stirred at room temperature for 3.0 h. Next, the mixture was poured into ice water and the solid was filtered out. The crude product was then purified by silica gel column chromatography (DCM/MeOH=10:1) to afford 2.5 g of the desired product as a white solid (66.00% yield). LC/MS: mass calcd. For C₄₄H₅₃N₁₇O₁₀: 979.42, found: 980.80 [M+H]⁺.

Step 12: The procedure was the same as 4-[3-[(tert-butoxycarbonyl)amino]propanamido]-1-methylimidazole-2-carboxylic acid, but the reaction temperature was 40° C. and reaction time was 4.0 h. 2.50 g of Ethyl 1-methyl-4-[3-([1-methyl-4-[1-methyl-4-(3-[[1-methyl-4-(1-methyl-4-[3-[(1-methylpyrrol-2-yl)formamido]propanamido]imidazole-2-amido)pyrrol-2-yl]formamido]propanamido)imidazole-2-amido]pyrrol-2-yl]formamido)propanamido]imidazole-2-carboxylate was used, 1.90 g of the desired product was observed as a white solid (78.00% yield). LC/MS: mass calcd. For C₄₂H₄₉N₁₇O₁₀: 951.38, found: 952.65 [M+H]⁺.

### Example 2. Synthesis of 3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanoic acid (PA-004)

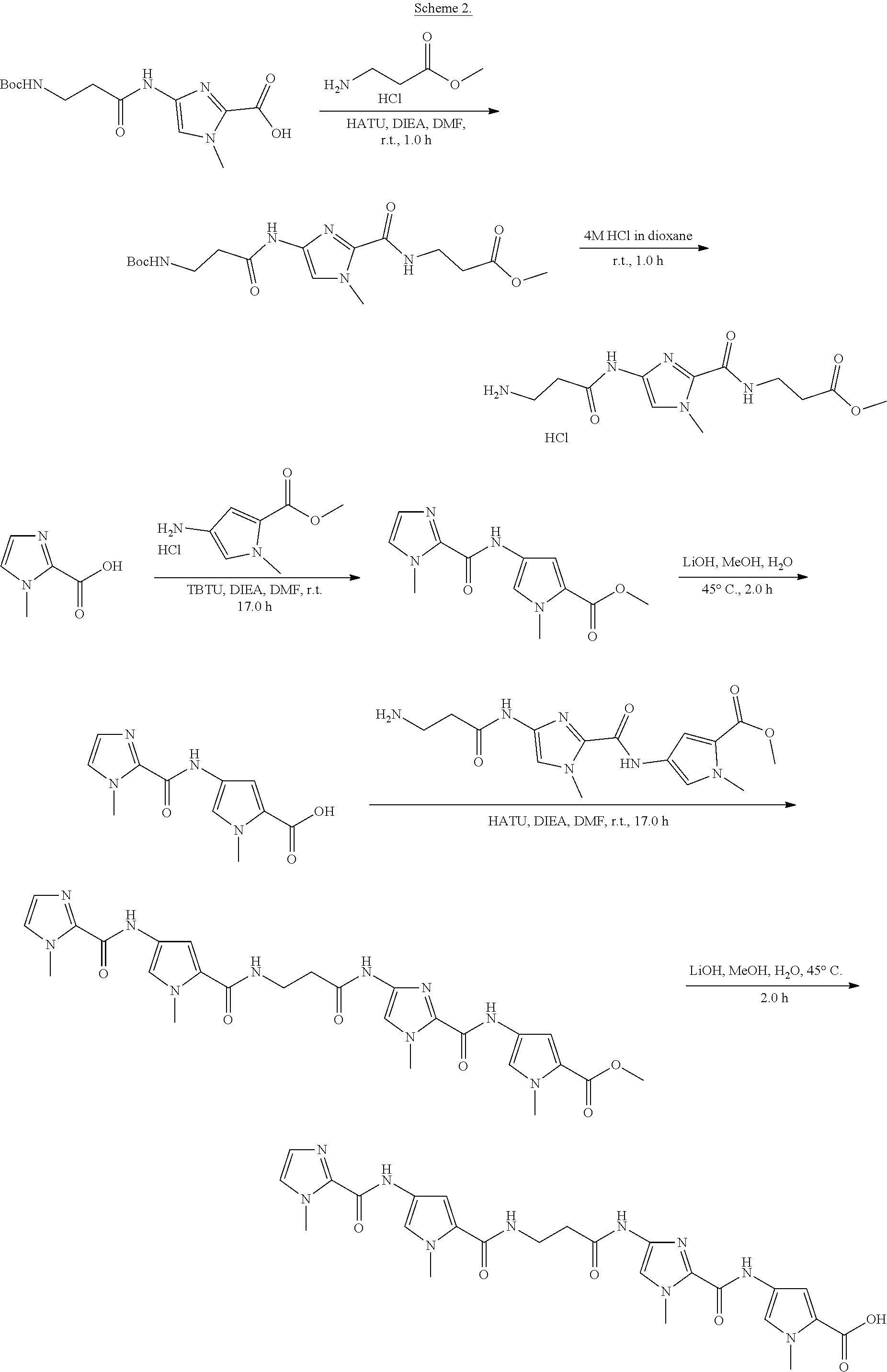

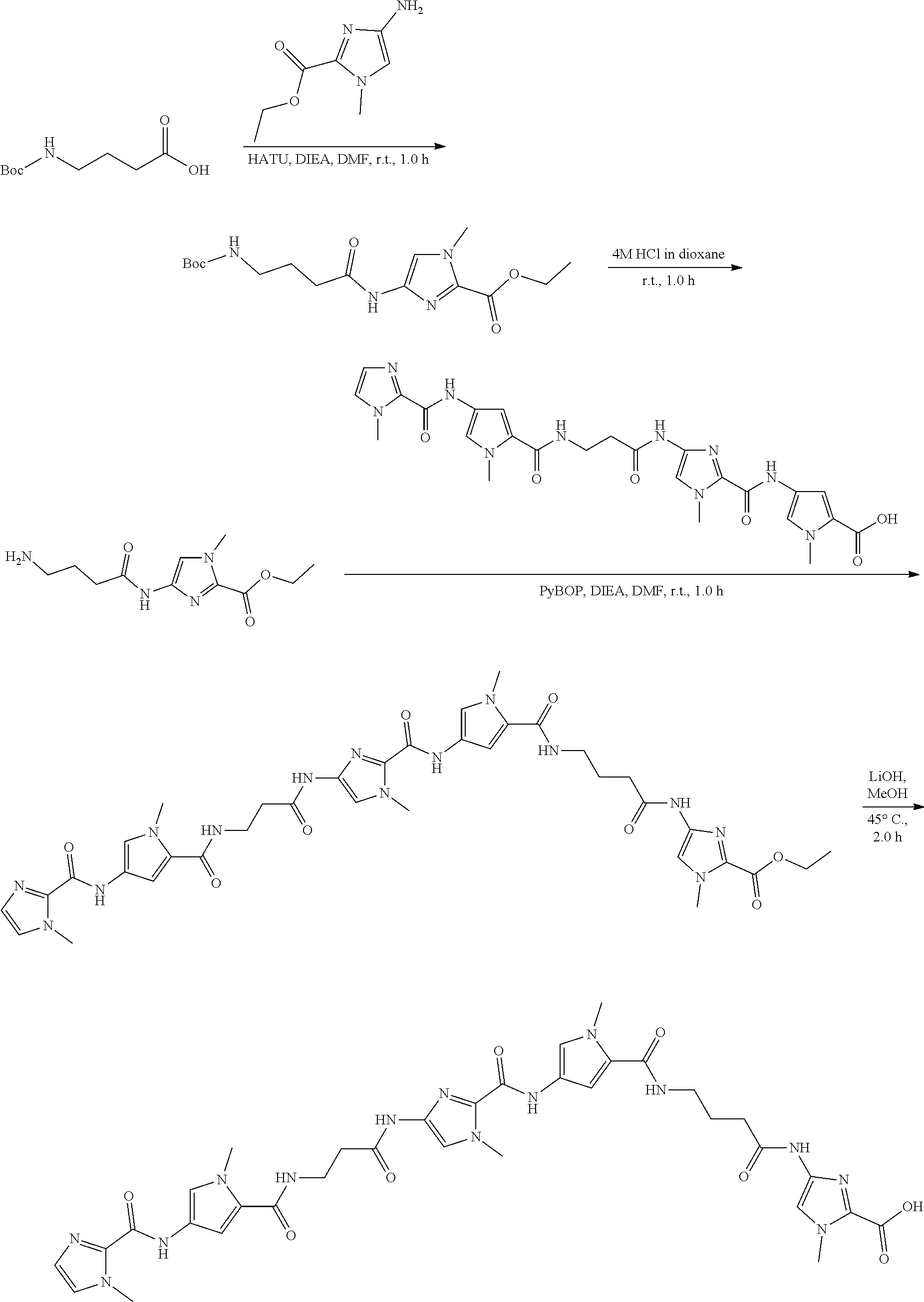

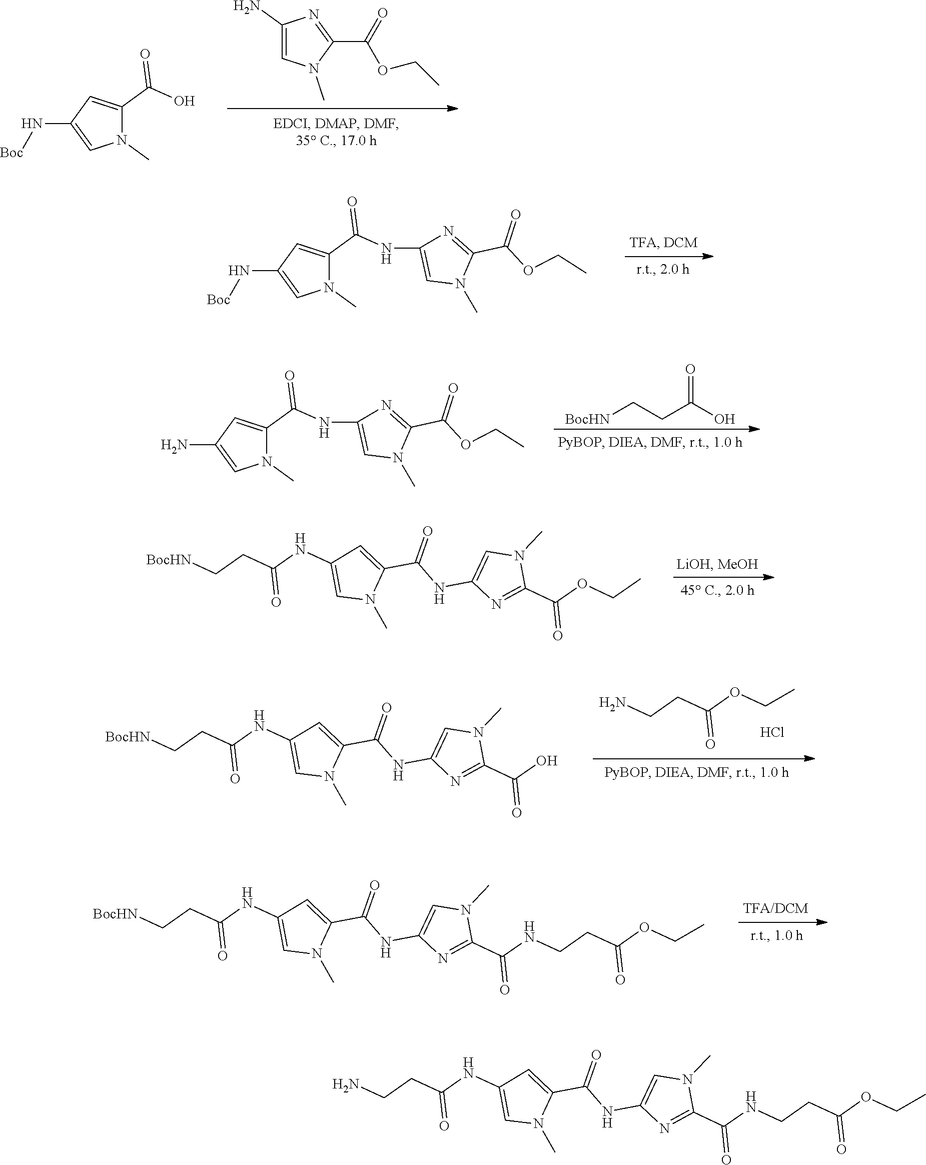

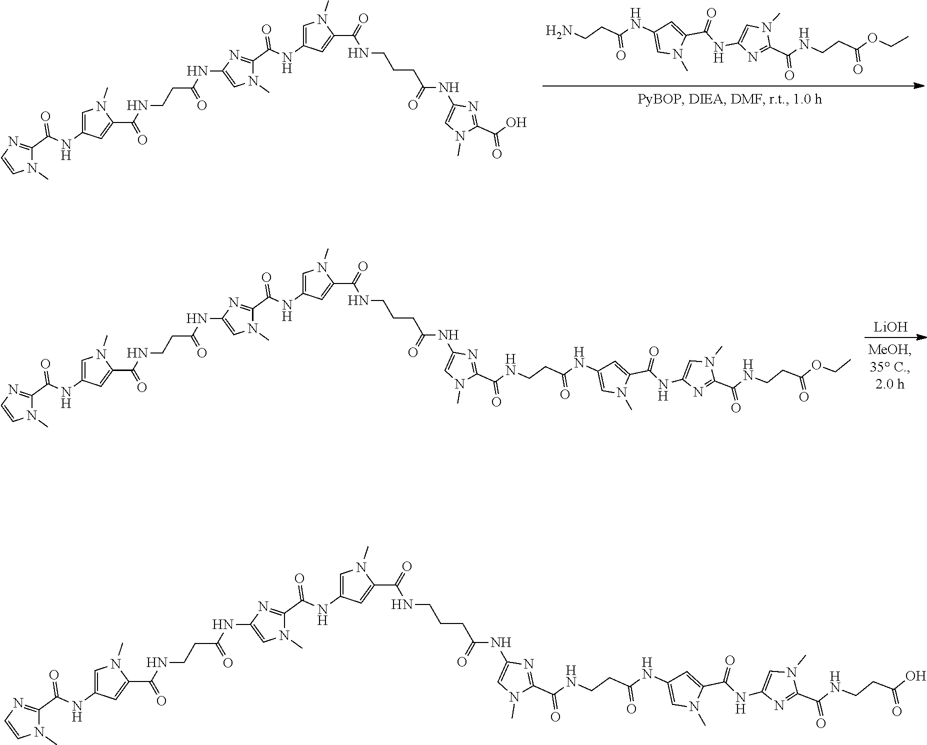 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)O)n1 ; CCOC(=O)CCNC(=O)c1nc(NC(=O)c2cc(NC(=O)CCN)cn2C)cn1C ; CCOC(=O)CCNC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C ; "LiOH" ; Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)O)n3)n(C)c2)n1]

Step 1: Into a 1000 ml flask was added 4-[3-[(tert-butoxycarbonyl)amino]propanamnido]-1-methylimidazole-2-carboxylic acid (11.00 g, 35.22 mmol, 1.00 equiv), and DMF (300.00 mL). The mixture was cooled to 0° C. and then HATU(20.09 g, 52.83 mmol, 1.50 equiv) and DIEA (18.21 g, 140.88 mmol, 4.00 equiv) were added dropwise. The resulting mixture was stirred for 10 mins and methyl 3-aminopropanoate (3.63 g, 35.22 mmol, 1.00 equiv) was added in portions. The reaction was stirred at room temperature for 1.0 h. Next the reaction mixture was poured into water/ice (600 mL) and the solid was filtered out and dried under vacuum. The aqueous phase was extracted with EA (3×200 mL) and the combined organic phase was washed with H₂O (1×200 mL) and NaCl (1×200 mL) and dried over anhydrous Na₂SO₄. After filtration, the filtrate was concentrated under reduced pressure. The residue was purified using a silica gel column and eluted with pure EA. The fractions were combined and concentrated to afford methyl 3-[(4-[3-[(tert-butoxy carbonyl)amino]propanamido]-1-methylimidazol-2-yl)formamido]propanoate (13.00 g, 87.95%) as a yellow solid. LC/MVS: mass calcd. For C₇H₂₇N₅O₆: 397.20, found: 398.20 [M+H]⁺.

Step 2: The procedure was the same as methyl 4-[4-(3-aminopropanamido)-1-methylimidazole-2-amido]-1-methylpyrrole-2-carboxylate hydrochloride (Example 1 step 6), but the reaction time was 1.0 h. Methyl 3-[(4-[3-[(tert-butoxycarbonyl)amino]propanamido]-1-methylimidazol-2-yl)formamido]propanoate (11.00 g) was used to obtain 11.00 g crude of the desired product as yellow oil. LC/MS: mass calcd. For C₁₂H₁₉N₅O₄: 297.14, found: 298.20 [M+H]⁺.

Step 3: To a stirred solution of 1-methylimidazole-2-carboxylic acid (10.00 g, 79.29 mmol, 7.00 equiv) in DMF (150.00 mL) was added TBTU (38.19 g, 118.94 mmol, 1.50 equiv), methyl 4-amino-1-methylpyrrole-2-carboxylate hydrochloride (16.63 g, 87.24 mmol, 1.10 equiv), and DIEA (30.74 g, 237.88 mmol, 3.00 equiv) in portions at 0° C. The resulting mixture was stirred for 17.0 h at room temperature. The reaction was poured into water/ice (450 mL) and the precipitated solids were collected by filtration, washed with H₂O (3×50 mL), and dried under vacuum. Methyl 1-methyl-4-(1-methylimidazole-2-amido)pyrrole-2-carboxylate (16.5 g, 78.37%) was obtained as a white solid. LC/MS: mass calcd. For C₁₂H₁₄N₄O₃: 262.11, found: 263.15 [M+H]⁺.

Step 4: The procedure was the same as 4-[3-[(tert-butoxycarbonyl)amino]propanamido]-1-methylimidazole-2-carboxylic acid (Example 1 step 3). Methyl 1-methyl-4-(1-methylimidazole-2-amido)pyrrole-2-carboxylate (16.50 g) was used to obtain 12.00 g of 1-methyl-4-(1-methylimidazole-2-amido)pyrrole-2-carboxylic acid (76.84% yield) as a white solid. LC/MS: mass calcd. For C₁₁H₁₂N₄O₃: 248.09, found: 249.10 [M+H]⁺.

Step 5: The procedure was the same as ethyl 3-[(4-[3-[(tert-butoxycarbonyl)amino]propanamido]-1-methylimidazol-2-yl)formamido]propanoate (Example 1 step 2). 1-Methyl-4-(1-methylimidazole-2-amido)pyrrole-2-carboxylic acid (9.00 mg) was used and 14.00 g of the desired product (63.54% yield) was obtained as yellow solid. LC/MS: mass calcd. For C₂₆H₃₀N₁₀O₆: 578.23, found: 579.10 [M+H]⁺.

Step 6: The procedure was the same as 4-[3-[(tert-butoxycarbonyl)amino]propanamido]-1-methylimidazole-2-carboxylic acid (Example 1 step 3). Methyl 1-methyl-4-[1-methyl-4-(3-[[1-methyl-4-(1-methylimidazole-2-amido) pyrrol-2-yl]formamido]propanamido)imidazole-2-amido]pyrrole-2-yl]formamidocarboxylate (14.00 g) was used, and 12.00 g of the desired product (81.49% yield) was obtained as a yellow solid. LC/MS: mass calcd. For C₂₅H₂₈N₁₀O₆: 564.22, found: 565.15[M+H]⁺.

Step 7: The procedure was the same as ethyl 4-[3-[(tert-butoxycarbonyl)amino]propanamido]-1-methylimidazole-2-carboxylate (Example 1 step 2). 4-[(Tert-butoxycarbonyl)amino]butanoic acid (7.80 g) was used and 11.00 g of the desired product was obtained as a pink solid (80.70% yield). LC/MS: mass calcd. For C₁₆H₂₆N₄O₅: 354.19, found: 355.15[M+H]⁺.

Step 8: The procedure was the same as methyl 4-[4-(3-aminopropanamido)-1-methylimidazole-2-amido]-1-methylpyrrole-2-carboxylate hydrochloride (Example 1 step 6). Ethyl 4-{4-[(tert-butoxycarbonyl)amino]butanamido}-1-methylimidazole-2-carboxylate (9.40 g) was used and 6.20 g of the desired product was obtained as a white solid (90.89% yield). LCMS: mass calcd. For C₁₁H₁₈N₄O₃: 254.14, found: 255.15[M+H]⁺.

Step 9: To a stirred solution of 1-methyl-4-[1-methyl-4-(3-{[1-methyl-4-(1-methylimidazole-2-amido)pyrrol-2-yl]formamido}propanamido)imidazole-2-amido]pyrrole-2-carboxylic acid (18.20 g, 32.24 mmol, 1.00 equiv) in DMF (250.00 mL) was added DIEA (12.50 g, 96.71 mmol, 3.00 equiv), ethyl 4-(4-aminobutanamido)-1-methylimidazole-2-carboxylate (9.02 g, 35.46 mmol, 1.10 equiv), and PyBOP (20.13 g, 38.68 mmol, 1.20 equiv) at 0° C. The resulting mixture was stirred for 1.0 h at room temperature and was then poured into ice/water (800 mL). The precipitated solids were collected by filtration, washed with H₂O (3×200 mL), and dried under vacuum to afford 24.70 g of ethyl 1-methyl-4-14-(1-methyl-4-|1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylate as a yellow solid (95.74% yield). LC/MS: mass calcd. For C₃₆H₄₄N₄O₈: 800.35, found: 801.30[M+H]⁺.

Step 10: The procedure was the same as 4-[3-[(tert-butoxycarbonyl)amino]propanamido]-1-methylimidazole-2-carboxylic acid (Example 1 step 3). Ethyl 1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylate (24.00 g) was used and 23.10 g of the desired product was obtained as a yellow solid (99.36% yield). LC/MS: mass calcd. For C₃₄H₄₀N₁₄O₈: 772.32, found: 773.30[M+H]⁺.

Step 11: To a stirred solution of 4-[(tert-butoxycarbonyl)amino]-1-methylpyrrole-2-carboxylic acid (11.50 g, 47.87 mmol, 1.00 equiv) in DMF (200.00 mL) was added EDCl (22.94 g, 119.66 mmol, 2.50 equiv), ethyl 4-amino-1-methylimidazole-2-carboxylate (8.10 g, 47.87 mmol, 1.00 equiv), and DMAP (14.62 g, 119.66 mmol, 2.50 equiv) at 0° C. The resulting mixture was stirred for 17.0 h at 35° C. The reaction was then poured into 500 mL ice/water and the precipitated solids were collected by filtration, washed with water (3×50 mL), and dried under vacuum. This resulted in ethyl 4-{4-[(tert-butoxycarbonyl)amino]-1-methylpyrrole-2-amido}-1-methylimidazole-2-carboxylate (16.00 g, 85.48% yield) as a light yellow solid. LC/MS: mass calcd. For C₁₈H₂₅N₅O₅: 391.19, found: 392.30 [M+H]⁺.

Step 12: To a stirred solution of ethyl 4-{4-[(tert-butoxycarbonyl)amino]-1-methylpyrrole-2-amido}-1-methylimidazole-2-carboxylate (16.00 g, 40.88 mmol, 1.00 equiv) in DCM (135.00 mL) was added TFA (45.00 mL) dropwise at room temperature. The resulting mixture was stirred for 2.0 h at room temperature and was hen concentrated under vacuum. The resulting brown oil was diluted with Et₂O (200 mL). The precipitated solids were collected by filtration, washed with Et₂O (2×100 mL), and dried under vacuum. This resulted in ethyl 4-(4-amino-1-methylpyrrole-2-amido)-1-methylimidazole-2-carboxylate (16.00 g, crude) as a brown solid. LC/MS: mass calcd. For C₁₃H₇N₅O₃: 291.13, found: 292.15[M+H]⁺.

Step 13: A solution of ethyl 4-(4-amino-1-methylpyrrole-2-amido)-1-methylimidazole-2-carboxylate (12.00 g, 41.19 mmol, 1.00 equiv) and 3-[(tert-butoxycarbonyl)amino]propanoic acid (7.50 g. 39.64 mmol, 0.96 equiv), PyBOP (22.00 g, 42.28 mmol. 1.03 equiv). DIEA (45.00 g, 348.18 mmol, 8.45 equiv) in DMF (120.00 mL) was stirred for 1.0 h at room temperature. The reaction was poured into ice water (400 mL) and the mixture was stirred for 15 min. The precipitated solids were collected by filtration, washed with water (3×150 mL), and dried under vacuum. The aqueous phase was extracted with EA (3×150 mL) and the combined organic phases were combined and washed by H₂O (200 mL) and dried over anhydrous Na₂SO₄. The solid was filtered out and the filtrate was concentrated. The residue was purified by silica gel column chromatography and eluted with PE/EA (1:8). This resulted in 17.00 g of ethyl 4-(4-{3-[(tert-butoxycarbonyl)amino]propanamido}-1-methylpyrrole-2-amido)-1-methylimidazole-2-carboxylate as a yellow solid (89.28% yield). LC/MS: mass calcd. For C₂₁H₃₀N₆O₆: 462.22, found: 463.35[M+H]⁺.

Step 14: The procedure was the same as 4-[3-[(tert-butoxycarbonyl)amino]propanamido]-1-methylimidazole-2-carboxylic acid (Example 1 step 3). Ethyl 4-(4-{3-1(tert-butoxycarbonyl)amino]propanamido}-1-methylpyrrole-2-amido)-1-methylimidazole-2-carboxylate (12.00 g) was used and 10.00 g of the desired product was obtained as a white solid (88.81% yield). LC/MS: mass calcd. For C₁₉H₂₆N₆O₆: 434.19, found: 435.25[M+H]⁺.

Step 15: A solution of 4-(4-{3-[(tert-butoxycarbonyl)amino]propanamido}-1-methylpyrrole-2-amido)-1-methylimidazole-2-carboxylic acid (10.00 g, 23.02 mmol, 1.00 equiv) and β-alanine ethyl ester hydrochloride (4.90 g, 31.90 mmol, 1.39 equiv), PyBOP (12.50 g, 24.02 mmol, 1.04 equiv), DIEA (9.00 g. 69.64 mmol, 3.03 equiv) in DMF (120.00 mL) was stirred for 1.0 h at room temperature. The reaction was quenched with water (500 mL) at room temperature and extracted with EA (3×400 mL). The combined organic layers were washed with brine (3×200 mL) and dried over anhydrous Na₂SO₄. The filtrate was concentrated under reduced pressure and the residue was purified using silica gel column chromatography and eluted with PE/EA (1:8) to afford ethyl 3-{[4-(4-{3-[(tert-butoxycarbonyl)amino]propanamido}-1-methylpyrrole-2-amido)-1-methylimidazol-2-yl]formamido}propanoate (12.00 g, 93.80%) as a yellow solid. LC/MS: mass calcd. For C₂₄H₃₅N₇O₇: 533.26, found: 534.30[M+H]⁺.

Step 16: The procedure was the same as ethyl 4-(4-amino-1-methylpyrrole-2-amido)-1-methylimidazole-2-carboxylate (Example 2 step 12). Ethyl 3-{[4-(4-{3-[(tert-butoxycarbonyl)amino]propanamido}-1-methylpyrrole-2-amido)-1-methylimidazol-2-yl]formamido}propanoate (12.00 g) was used and 12.00 g crude of the desired product was obtained as a white solid. LC/MS: mass calcd. For C₁₉H₂₇N₇O₅: 433.21, found: 434.25[M+H]⁺.

Step 17: The procedure was the same as ethyl 1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylate (Example 2 step 12). 1-Methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylic acid (10.00 g) was used and 13.60 g of the desired product was obtained as a yellow solid (88.61% yield). The derided product was obtained as a light yellow solid after purification by Prep-HPLC. HRMS: mass calcd. For C₅₃H₆₅N₂₁O₁₂: 1187.5122, found: 1188.5153[M+H]⁺.

Step 18: The procedure was the same as 4-[3-[(tert-butoxycarbonyl)amino]propanamido]-1-methylimidazole-2-carboxylic acid (Example 1 step 3), but the reaction temperature was 35° C. Ethyl 3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanoate (10.60 g) was used and 10.00 g of the desired product was obtained as a yellow solid. LC/MS: mass calcd. For C₅₁H₆₁N₂₁O₁₂: 1159.48, found: 581.25[M/2+H]⁺.

### Example 3. Synthesis of 1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido)pyrrol-2-yl}formamido)butanamido]imidazol-2-yl}formamido)propanamido|pyrrole-2-amido}imidazole-2-carboxylic acid (PA-040-OH)

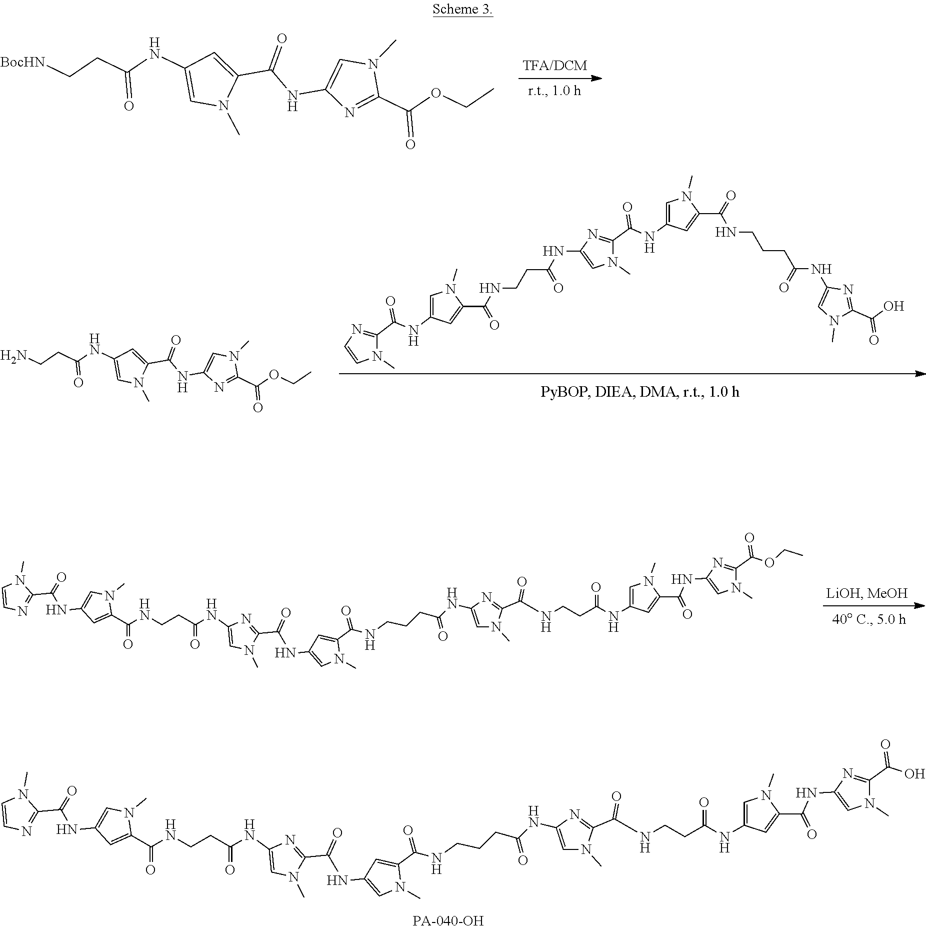 [SMILES: CCOC(=O)c1nc(NC(=O)c2cc(NC(=O)CCNC(=O)OC(C)(C)C)cn2C)cn1C ; CCOC(=O)c1nc(NC(=O)c2cc(NC(=O)CCN)cn2C)cn1C ; Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)O)n1 ; CCOC(=O)c1nc(NC(=O)c2cc(NC(=O)CCNC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C ; Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)O)n3)n(C)c2)n1]

Step 1: The procedure was the same as ethyl 4-(4-amino-1-methylpyrrole-2-amido)-1-methylimidazole-2-carboxylate (Example 2 step 12). Ethyl 4-(4-{3-[(tert-butoxycarbonyl)amino]propanamido}-1-methylpyrrole-2-amido)-1-methylimidazole-2-carboxylate (2.00 g) was used and 2.00 g of the desired product was obtained as a white solid. LC/MS: mass calcd. For C₁₆H₂₂N₆O₄: 362.17, found: 363.25[M+H]⁺.

Step 2: The procedure was the same as ethyl 1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylate (Example 2 step 9), but the solvent was DMA. 1-Methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylic acid (3.00 g) was used and 4.30 g of the desired product was obtained as a yellow solid (96.84% yield). LC/MS: mass calcd. For C₅₀H₆₀N₂₀O₁₁: 1116.48, found: 1117.60[M+H]⁺.

Step 3: The procedure was the same as 4-[3-[(tert-butoxycarbonyl)amino]propanamido]-1-methylimidazole-2-carboxylic acid (Example 1 step 3), but the reaction temperature was 40° C. and the reaction time was 5.0 h. Ethyl 1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido pyrrol-2-yl}formamido)butanamido imidazol-2-yl}formamido)propanamido pyrrole-2-amido}imidazole-2-carboxylate (4.20 g) was used and 4.00 g of the desired product was obtained as a yellow solid (97.97% yield). LC/MS: mass calcd. For C₄₃H₅₆N₂₀O₁₁: 1088.44, found: 1089.55[M+H]⁺.

### Example 4. Synthesis of 3-[(4-14-[3-(14-[(2R)-2-[(tert-butoxycarbonyl)amino]-4-({1-methyl-4-[1-methyl-4-(3-1[1-methyl-4-(1-methylimidazole-2-amido)pyrrol-2-yl]formamido}propanamido)imidazole-2-amido)pyrrol-2-yl}formamido)butanamido}-1-methylimidazol-2-yl}formamido)propanamido]-1-methylpyrrole-2-amidol-1-methylimidazol-2-yl)formamido]propanoic acid (PA-004-NHBoc-OH)

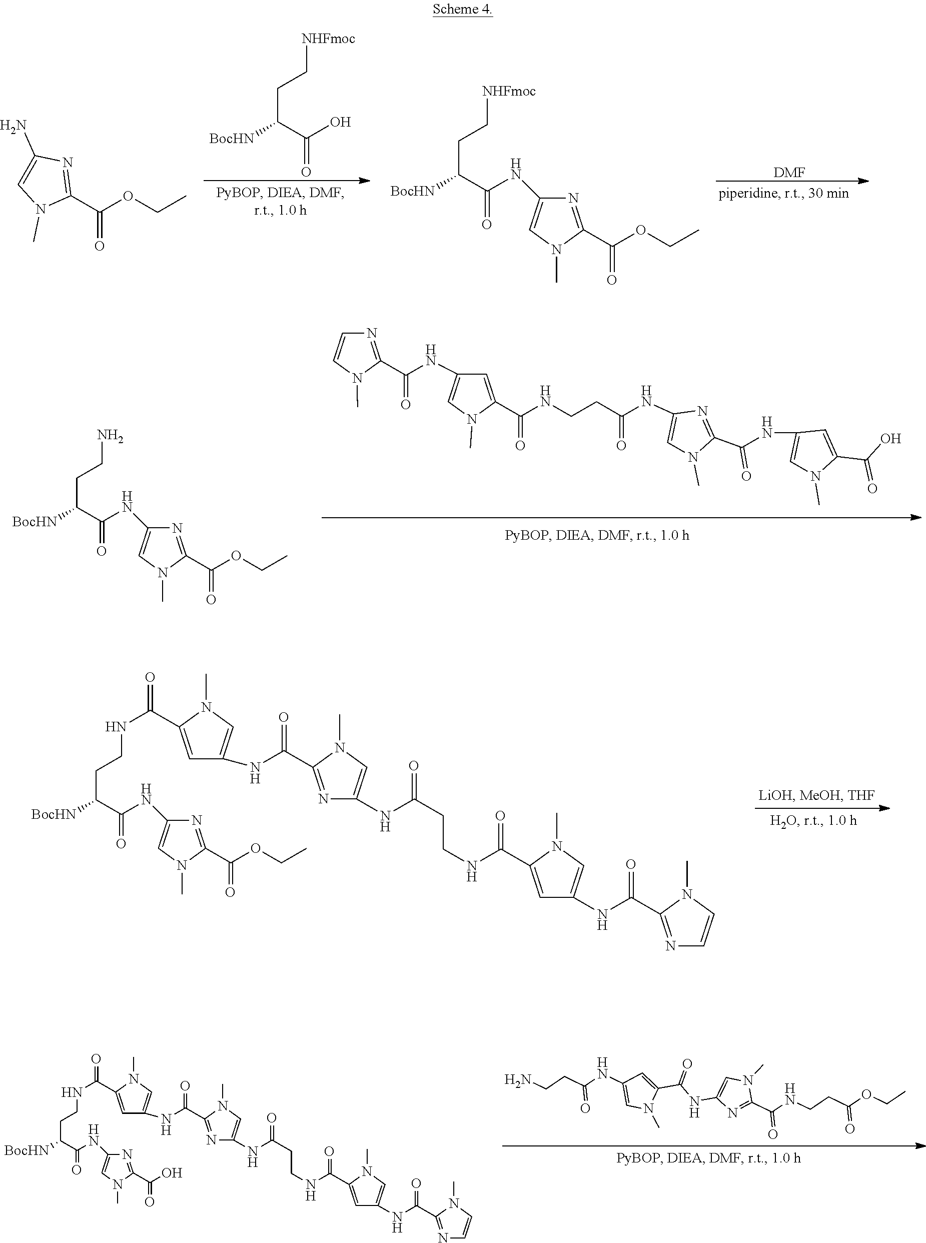

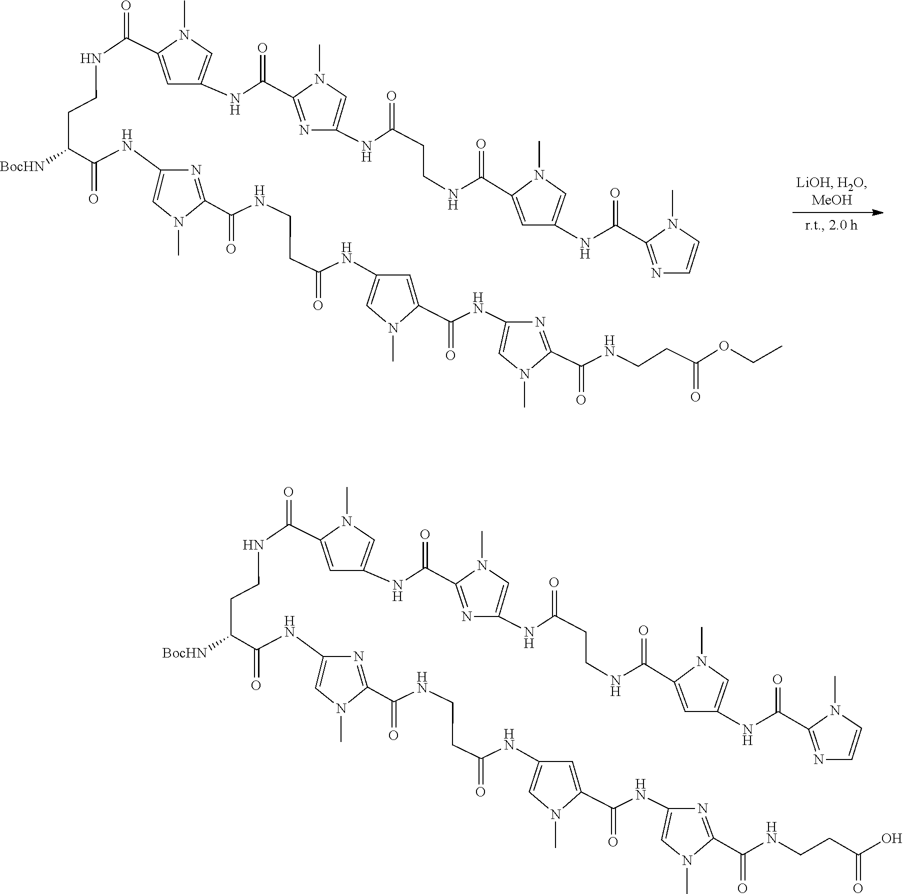 [SMILES: CCOC(=O)CCNC(=O)c1nc(NC(=O)c2cc(NC(=O)CCNC(=O)c3nc(NC(=O)[C@@H](CCNC(=O)c4cc(NC(=O)c5nc(NC(=O)CCNC(=O)c6cc(NC(=O)c7nccn7C)cn6C)cn5C)cn4C)NC(=O)OC(C)(C)C)cn3C)cn2C)cn1C ; Cn1cc(NC(=O)c2nccn2C)cc1C(=O)NCCC(=O)Nc1cn(C)c(C(=O)Nc2cc(C(=O)NCC[C@@H](NC(=O)OC(C)(C)C)C(=O)Nc3cn(C)c(C(=O)NCCC(=O)Nc4cc(C(=O)Nc5cn(C)c(C(=O)NCCC(=O)O)n5)n(C)c4)n3)n(C)c2)n1]

Step 1: The procedure was the same as ethyl 1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylate (Example 2 step 9). (2R)-2-[(tert-butoxycarbonyl)amino]-4-{[(9H-fluoren-9-ylmethoxy)carbonyl]amino}butanoic acid (3.00 g) was used and 3.50 g of the desired product was obtained as a yellow solid. LC/MS: mass calcd. For C₃₁H₃N₅O₇: 591.27, found: 592.25[M+H]⁺.

Step 2: Into a 50 ml flask was added ethyl 4-[(2R)-2-[(tert-butoxycarbonyl)amino]-4-{[(9H-fluoren-9-ylmethoxy)carbonyl]amino}butanamido]-1-methylimidazole-2-carboxylate (500.00 mg, 0.85 mmol, 1.00 equiv), DMF (5.00 mL), and piperidine (1.00 mL). The reaction was stirred at room temperature for 30 mins. The piperidine was removed and the reaction mixture was purified by reverse flash chromatography with the following conditions: Column, C18 column; Mobile Phase, CH₃CN in water (0.5% NH₄HCO₃), 10% to 50% gradient in 30 min; Detector, UV 254 nm. The fractions were combined and concentrated to afford ethyl 4-[(2R)-4-amino-2-[(tert-butoxycarbonyl)amino]butanamido]-1-methylimidazole-2-carboxylate (250.00 mg, 96.10%) as a yellow oil. LC/MS: mass calcd. For C₁₆H₂₇N₅O₅: 369.20, found: 370.35[M+H]⁺.

Step 3: The procedure was the same as ethyl 1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylate (Example 2 step 9). Ethyl 4-[(2R)-4-amino-2-[(tert-butoxycarbonyl)amino]butanamido]-1-methylimidazole-2-carboxylate (200.00 mg) was used and 200.00 mg of the desired product was obtained as a yellow solid (68.39% yield). LC/MS: mass calcd. For C₄₁H₅₃N₁₅O₁₀: 915.41, found: 916.75[M+H]⁺.

Step 4: The procedure was the same as 4-[3-[(tert-butoxycarbonyl)amino]propanamido]-1-methylimidazole-2-carboxylic acid (Example 1 step 3), but the reaction solvent was a mixture of MeOH/THF (5:3), the reaction temperature was room temperature, and the reaction time was 1.0 h. Ethyl 4-[(2R)-2-[(tert-butoxycarbonyl) amino]-4-({1-methyl-4-[1-methyl-4-(3-{[1-methyl-4-(1-methylimidazole-2-amido) pyrrol-2-yl]formamido}propanamido)imidazole-2-amido]pyrrol-2-yl}formamido)butanamido]-1-methylimidazole-2-carboxylate (570 mg) was used and 370.00 mg of the desired product was obtained as a yellow solid (66.90% yield). LC/MS: mass calcd. For C₃₉H₄₉N₁₅O₁₀: 887.38, found: 888.85[M+H]⁺.

Step 5: The procedure was the same as ethyl 1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylate (Example 2 step 9). 4-[(2R)-2-[(tert-butoxycarbonyl)amino]-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-1-methylimidazole-2-carboxylic acid (380.00 mg) was used and 521.00 mg of the desired product was obtained as a white solid (91.75% yield). The desired compound was obtained as a white solid after purification by Prep-HPLC. HRMS: mass calcd. For C₅₈H₇₄N₂₂O₁₄: 1302.5755, found: 1303.5867[M+H]⁺.

Step 6: The procedure was the same as 4-[3-[(tert-butoxycarbonyl)amino]propanamido]-1-methylimidazole-2-carboxylic acid (Example 1 step 3), but the reaction temperature was room temperature. Ethyl 3-[(4-{4-[3-({4-[(2R)-2-[(tert-butoxycarbonyl)amino]-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formanido)butananido]-1-methylimidazol-2-yl}formaimido)propanamido]-1-methylpyrrole-2-amido}-1-methylimidazol-2-yl)formamido]propanoate (150.00 mg) was used and 146.00 mg the desired product was obtained as a white solid. LC/MS: mass calcd. For C₅₆H₇₀N₂₂O₁₄ 1274.54 found: 638.85[M/2+H]⁺.

### Example 5. Synthesis of 3-[(4-{4-[3-({4-[(2R)-2-acetamido-4-({1-methyl-4-[1-methyl-4-(3-{[1-methyl-4-(1-methylimidazole-2-amido)pyrrol-2-yl}formamido}propanamido)imidazole-2-amido]pyrrol-2-yl}formamido)butanamido]-1-methylimidazol-2-yl}formamido)propanamido]-1-methylpyrrole-2-amidol-1-methylimidazol-2-yl)formamido]propanoic acid (PA-004-NHAc—OH)

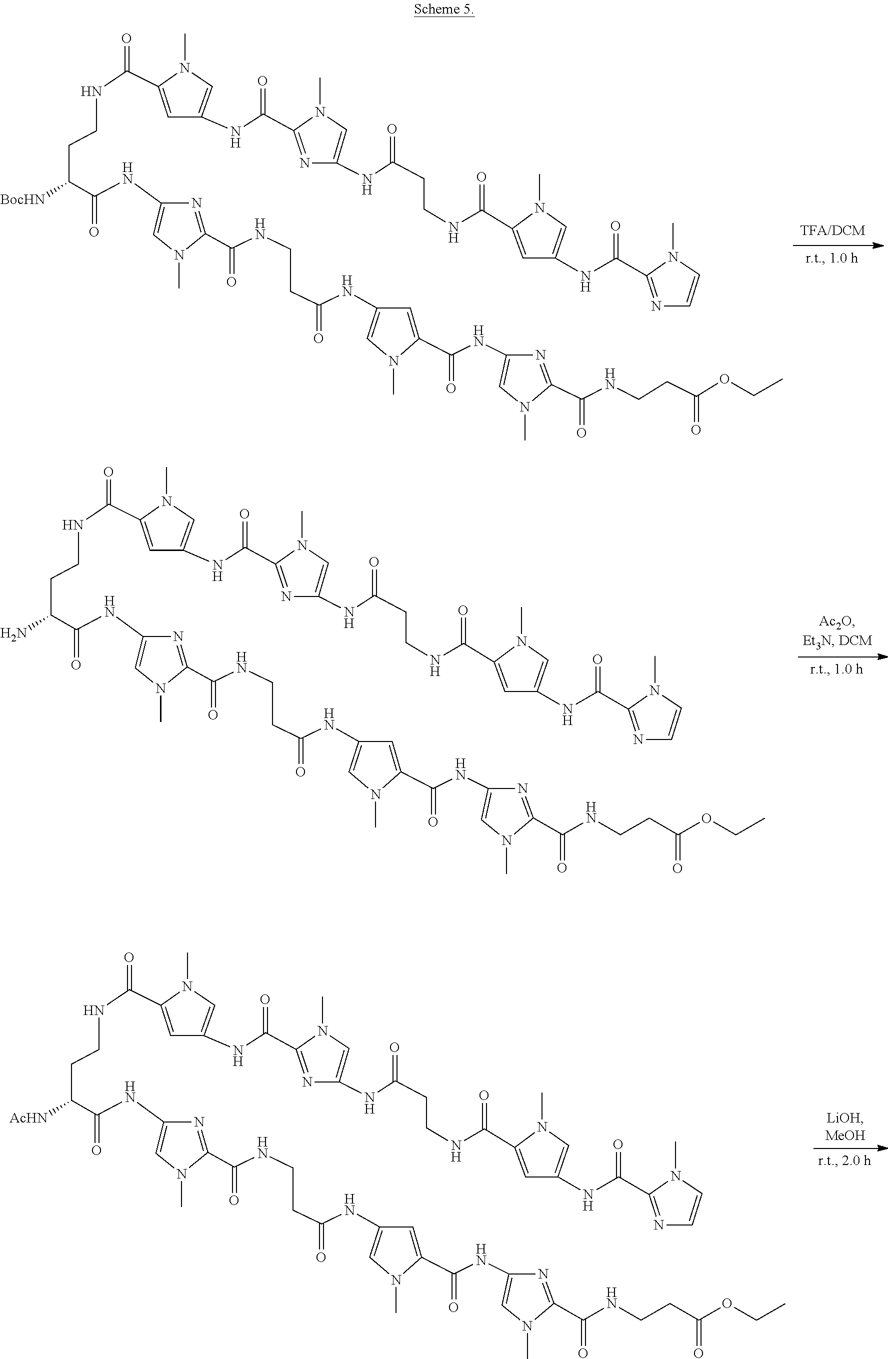 [SMILES: CCOC(=O)CCNC(=O)c1nc(NC(=O)c2cc(NC(=O)CCNC(=O)c3nc(NC(=O)[C@@H](CCNC(=O)c4cc(NC(=O)c5nc(NC(=O)CCNC(=O)c6cc(NC(=O)c7nccn7C)cn6C)cn5C)cn4C)NC(=O)OC(C)(C)C)cn3C)cn2C)cn1C ; CCOC(=O)CCNC(=O)c1nc(NC(=O)c2cc(NC(=O)CCNC(=O)c3nc(NC(=O)[C@H](N)CCNC(=O)c4cc(NC(=O)c5nc(NC(=O)CCNC(=O)c6cc(NC(=O)c7nccn7C)cn6C)cn5C)cn4C)cn3C)cn2C)cn1C ; CCOC(=O)CCNC(=O)c1nc(NC(=O)c2cc(NC(=O)CCNC(=O)c3nc(NC(=O)[C@@H](CCNC(=O)c4cc(NC(=O)c5nc(NC(=O)CCNC(=O)c6cc(NC(=O)c7nccn7C)cn6C)cn5C)cn4C)NC(C)=O)cn3C)cn2C)cn1C]

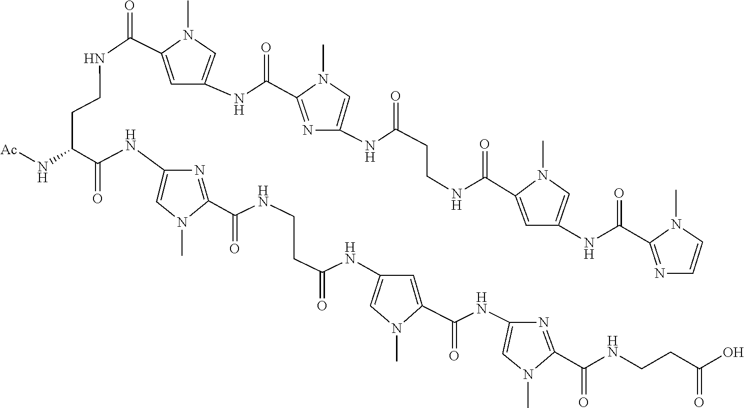 [SMILES: CC(=O)N[C@H](CCNC(=O)c1cc(NC(=O)c2nc(NC(=O)CCNC(=O)c3cc(NC(=O)c4nccn4C)cn3C)cn2C)cn1C)C(=O)Nc1cn(C)c(C(=O)NCCC(=O)Nc2cc(C(=O)Nc3cn(C)c(C(=O)NCCC(=O)O)n3)n(C)c2)n1]

Step 1: The procedure was the same as ethyl 4-(4-amino-1-methylpyrrole-2-amido)-1-methylimidazole-2-carboxylate (Example 2 step 12), but after concentration the crude was used directly in the next step without further purification. Ethyl 3-[(4-{4-[3-({4-[(2R)-2-amino-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-1-methylimidazol-2-yl}formamido)propanamido]-1-methylpyrrole-2-amido}-1-methylimidazol-2-yl)formamido]propanoate (319.00 mg) was used and 300.00 mg of the desired product was obtained as a brown solid. After purification by Prep-HPLC, the desired compound was obtained as white solid. HRMS: mass calcd. For C₅₃H₆₆N₂₂O₁₂: 1202.5231, found: 1203.5302[M+H]⁺.

Step 2: To a stirred solution of ethyl 3-[(4-{4-[3-({4-[(2R)-2-amino-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-1-methylimidazol-2-yl}formamido)propanamido]-1-methylpyrrole-2-amido}-1-methylimidazol-2-yl)formamido]propanoate (294.50 mg, 0.25 mmol, 1.00 equiv) in DCM (6.00 mL) was added Ac₂O (0.23 mL, 2.45 mmol, 10.00 equiv) and Et₃N (0.34 mL, 2.45 mmol, 10.00 equiv) in portions at 0° C. The resulting mixture was stirred for 1.0 h at room temperature and was then concentrated under vacuum. The crude product was purified by reverse phase column with the following condition: Column. C18; Mobile Phase, ACN in water (0.5% TFA), 10% to 50% gradient in 30 min; Detector, UV 254 nm. The factions were combined and lyophilized to afford ethyl 3-[(4-{4-[3-({4-[(2R)-2-acetamido-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-1-methylimidazol-2-yl}formamido)propanamido]-1-methylpyrrole-2-amido}-1-methylimidazol-2-yl)formamido]propanoate (200.00 mg, 64.31%) as a white solid. LC/MS: mass calcd. For C₅₅H₆₈N₂₂O₁₃: 1244.5336, found: 1245.5392[M+H]⁺.

Step 3: The procedure was the same as 4-[3-[(tert-butoxycarbonyl)amino]propanamido]-1-methylimidazole-2-carboxylic acid (Example 1 step 3), but the reaction temperature was room temperature. Ethyl (R)-3-(4-(4-(3-(4-(2-acetamido-4-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)butanamido)-1-methyl-1H-imidazole-2-carboxamido)propanamido)-1-methyl-1H-pyrrole-2-carboxamido)-1-methyl-1H-imidazole-2-carboxamido) propanoate (190.00 mg) was used and 91.50 mg of the desired product was obtained as a white solid (50.12% yield). LC/MS: mass calcd. For C₅₃H₆₄N₂₂O₁₃: 1216.50, found: 609.80 [M/2+H]⁺.

### Example 6. Synthesis of 1-methyl-4-(1-methyl-4-13-[(1-methyl-4-{11-methyl-4-[4-({1-methyl-4-[1-methyl-4-(3-{[l-methyl-4-(1-methylimidazole-2-amido)pyrrol-2-yl]formamido}propanamido)imidazole-2-amidol pyrrol-2-yl}formamido)butanamido]imidazole-2-amido}pyrrol-2-yl)formamido]propanamido}imidazole-2-amido)pyrrole-2-carboxylic acid (PA-049-OH)

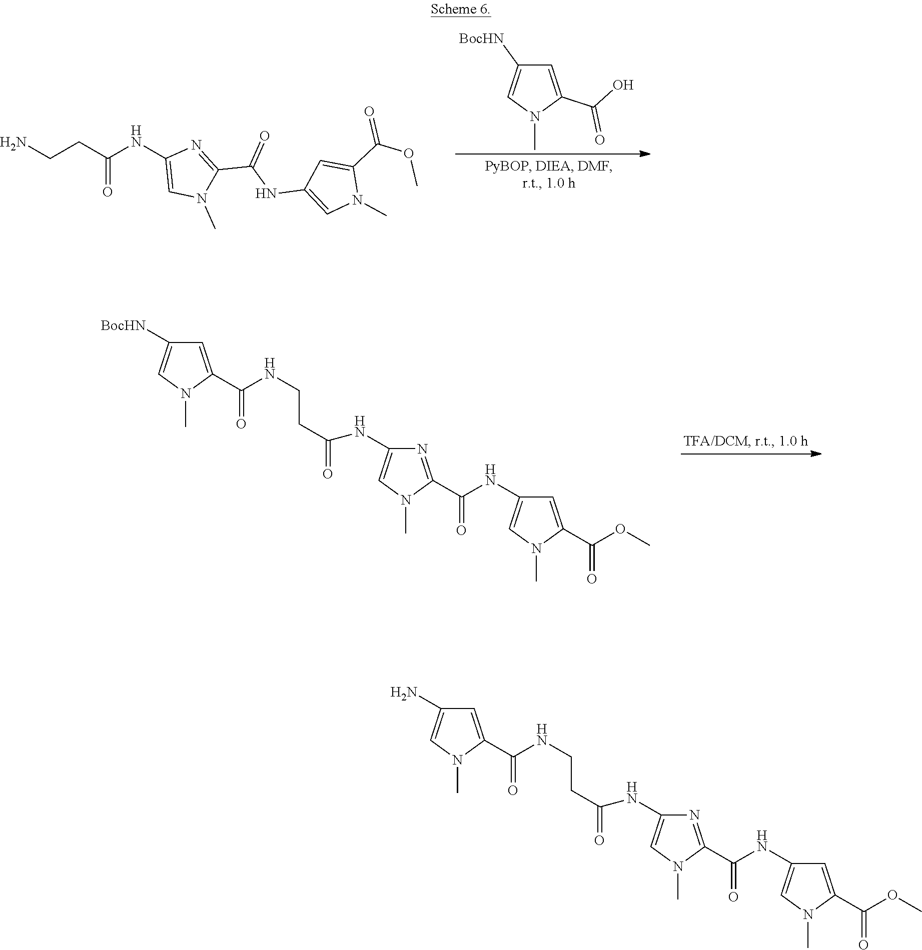 [SMILES: COC(=O)c1cc(NC(=O)c2nc(NC(=O)CCN)cn2C)cn1C ; Cn1cc(NC(=O)OC(C)(C)C)cc1C(=O)O ; COC(=O)c1cc(NC(=O)c2nc(NC(=O)CCNC(=O)c3cc(NC(=O)OC(C)(C)C)cn3C)cn2C)cn1C ; COC(=O)c1cc(NC(=O)c2nc(NC(=O)CCNC(=O)c3cc(N)cn3C)cn2C)cn1C]

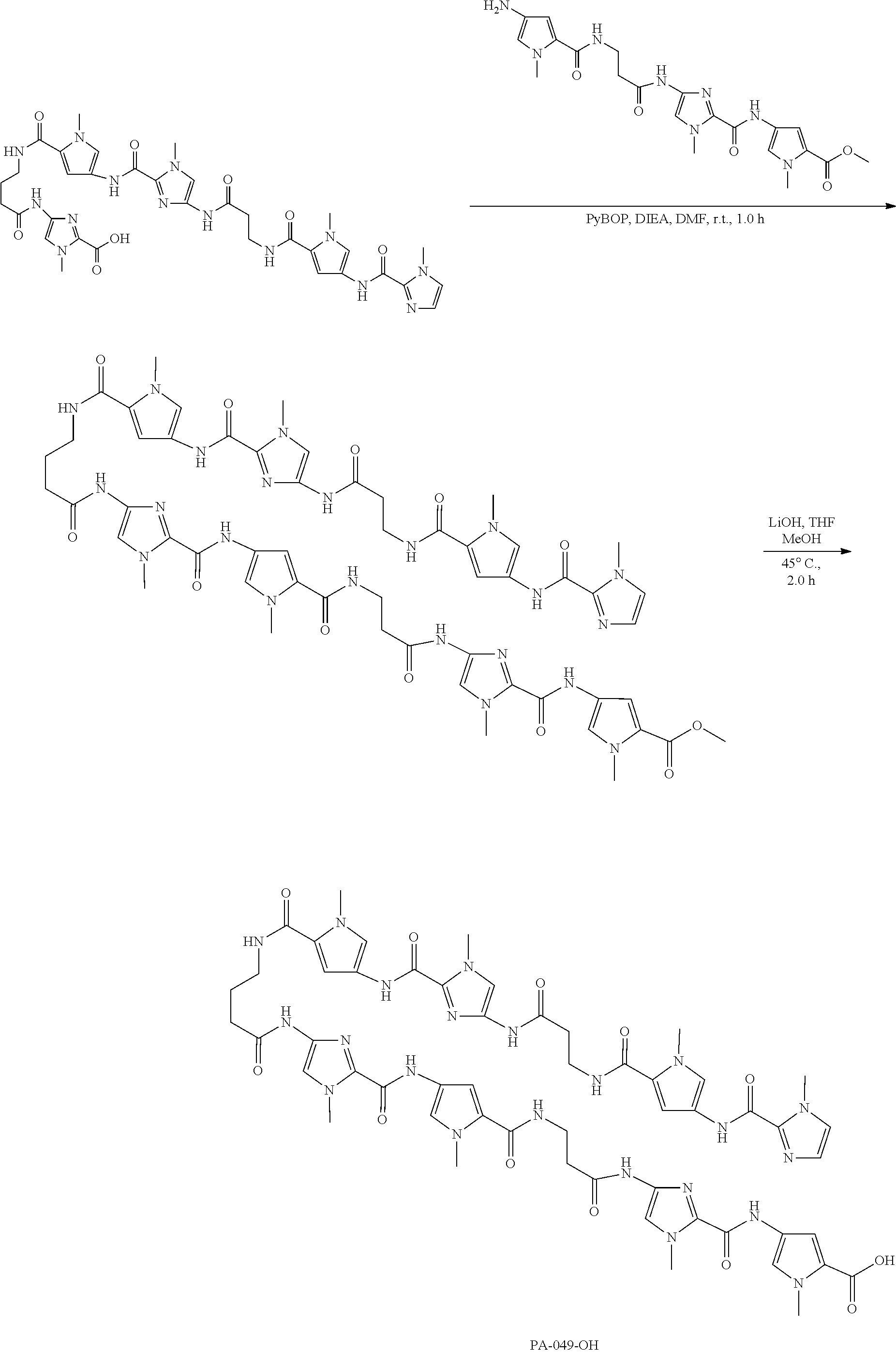 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)O)n1 ; COC(=O)c1cc(NC(=O)c2nc(NC(=O)CCNC(=O)c3cc(N)cn3C)cn2C)cn1C ; COC(=O)c1cc(NC(=O)c2nc(NC(=O)CCNC(=O)c3cc(NC(=O)c4nc(NC(=O)CCCNC(=O)c5cc(NC(=O)c6nc(NC(=O)CCNC(=O)c7cc(NC(=O)c8nccn8C)cn7C)cn6C)cn5C)cn4C)cn3C)cn2C)cn1C ; Cn1cc(NC(=O)c2nc(NC(=O)CCNC(=O)c3cc(NC(=O)c4nc(NC(=O)CCCNC(=O)c5cc(NC(=O)c6nc(NC(=O)CCNC(=O)c7cc(NC(=O)c8nccn8C)cn7C)cn6C)cn5C)cn4C)cn3C)cn2C)cc1C(=O)O]

Step 1: The procedure was the same as ethyl 1-methyl-4-[4-({1-methyl-4-[1-methyl-4-(3-{[1-methyl-4-(1-methylimidazole-2-amido)pyrrol-2-yl]formamido}propanamido) imidazole-2-amido]pyrrol-2-yl}formamido)butanamido]imidazole-2-carboxylate (Example 2 step 9). 4-[(Tert-butoxycarbonyl)amino]-1-methylpyrrole-2-carboxylic acid (2.07 g) was used and 3.00 g of the desired product was obtained as a yellow solid. LC/MS: mass calcd. For C₂₆H₃₄N₈O₇: 570.26, found: 571.30 [M+H]⁺.

Step 2: The procedure was the same as ethyl 4-(4-amino-1-methylpyrrole-2-amido)-1-methylimidazole-2-carboxylate (Example 2 step 12), but after concentration the crude was used directly in the next step without further purification. Methyl 4-{4-[3-({4-[(tert-butoxycarbonyl)amino]-1-methylpyrrol-2-yl}formamido)propanamido]-1-methylimidazole-2-amido}-1-methylpyrrole-2-carboxylate (355.00 g) was used and 350.00 mg of methyl 4-(4-{3-[(4-amino-1-methylpyrrol-2-yl)formamido]propanamido}-1-methylimidazole-2-amido)-1-methylpyrrole-2-carboxylate was obtained as a brown oil. LC/MS: mass calcd. For C₂₁H₂₆N₈O₅: 470.20, found: 471.45[M+H]⁺.

Step 3: The procedure was the same as ethyl 1-methyl-4-[4-({1-methyl-4-[1-methyl-4-(3-{[1-methyl-4-(1-methylimidazole-2-amido)pyrrol-2-yl]formamido}propanamido) imidazole-2-amido]pyrrol-2-yl}formamido)butanamido]imidazole-2-carboxylate (Example 2 step 9). 1-Methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylic acid (450.00 g) was used and 790.00 mg of the desired product was obtained as a white solid (95.77% yield). LC/MS: mass calcd. For C₅₅H₆₄N₂₂O₂:1224.51, found: 1225.85 [M+H]f.

Step 4: The procedure was the same as 4-[3-[(tert-butoxycarbonyl)amino]propanamido]-1-methylimidazole-2-carboxylic acid (Example 1 step 3), but the reaction solvent was MeOH/THF=1:1. Methyl 1-methyl-4-(1-methyl-4-{3-[(1-methyl-4-{1-methyl-4-[4-({1-methyl-4-[1-methyl-4-(3-{[1-methyl-4-(1-methylimidazole-2-amido)pyrrol-2-yl|formamido}propanamido)imidazole-2-amido pyrrol-2-yl}formamido)butanamido]imidazole-2-amido}pyrrol-2-yl)formamido]propanamido}imidazole-2-amido)pyrrole-2-carboxylate (300.00 mg) was used, and 290.00 mg of the desired product was obtained as a white solid (68.45% yield). LC/MS: mass calcd. For C₅₄H₆₂N₂₂O₁₂:1210.49, found: 1211.80 [M+H]⁺.

### Example 7. Synthesis of 4-{3-[(4-{4-[(2R)-2-[(tert-butoxycarbonyl)amino]-4-[(1-methyl-4-{1-methyl-4-[1-methyl-4-(1-methylpyrrole-2-amido)pyrrole-2-amido}imidazole-2-amido}pyrrol-2-yl)formamido]butanamido]-1-methylimidazole-2-amidol-1-methylpyrrol-2-yl)formamido]propanamido]-1-methylimidazole-2-carboxylic acid (PA-044-NHBoc)

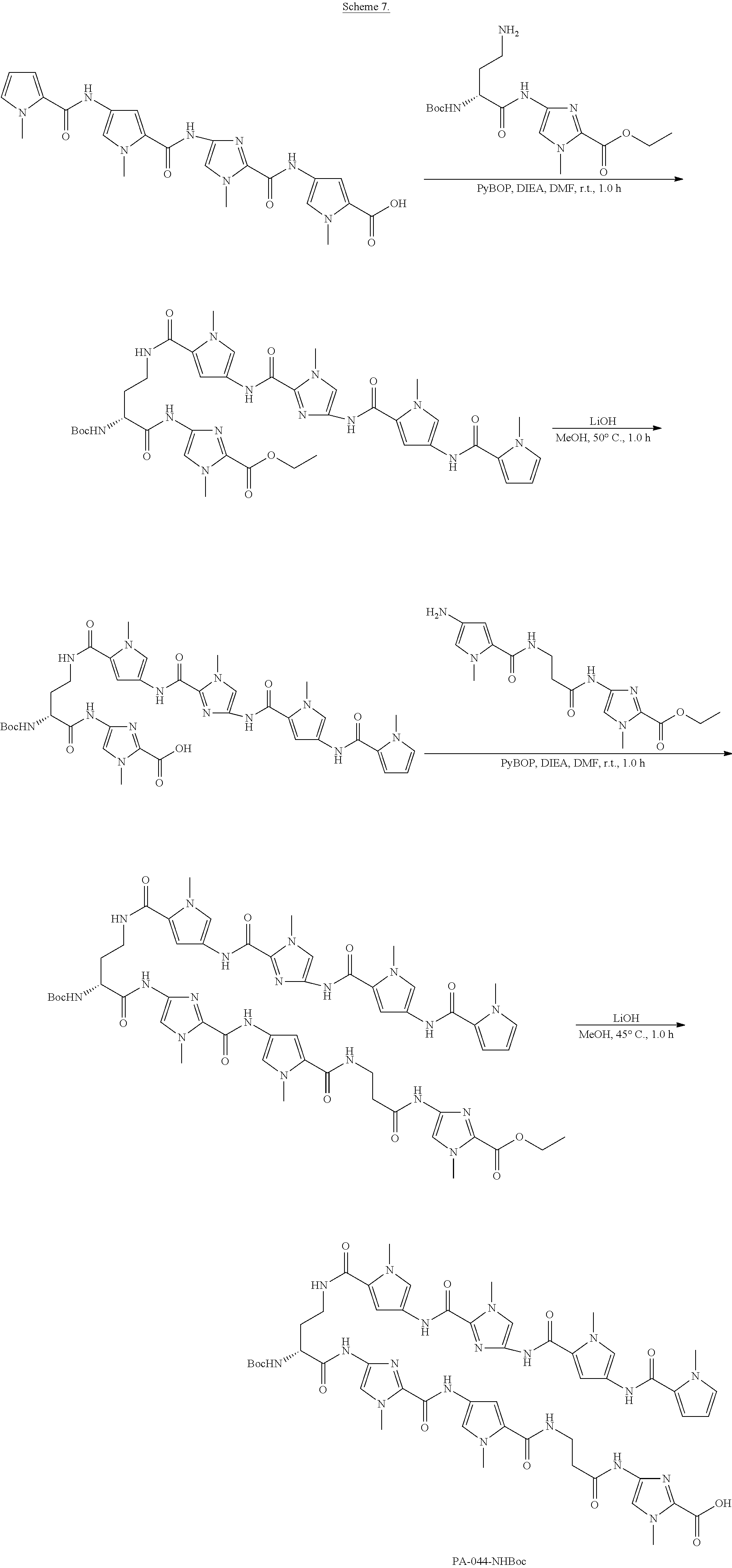

Step 1: The procedure was the same as ethyl 1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylate (Example 2 step 9). Ethyl 4-[(2R)-4-amino-2-[(tert-butoxycarbonyl)amino]butanamido]-1-methylimidazole-2-carboxylate (250.00 mg) was used, and 470.00 mg of the desired product was obtained as a white solid (82.30% yield). LC/MS: mass calcd. For C₃₉H₄₉N₁₃O₉: 843.38, found: 844.70 [M+H]⁺.

Step 2: The procedure was the same as 4-[3-[(tert-butoxycarbonyl)amino]propanamido]-1-methylimidazole-2-carboxylic acid (Example 1 step 3), but the reaction temperature was 50° C. and the reaction time was 1.0 h. Ethyl 4-[(2R)-2-[(tert-butoxycarbonyl)amino]-4-[(1-methyl-4-{1-methyl-4-[1-methyl-4-(1-methylpyrrole-2-amido)pyrrole-2-amido]imidazole-2-amido}pyrrol-2-yl)formamido]butanamido]-1-methylimidazole-2-carboxylate (460.00 mg) was used, and 370.00 mg of the desired product was obtained as a white solid (83.20% yield). LC/MS: mass calcd. For C₀H₄₅N O₉: 815.35, found: 816.60 [M+H]⁺.

Step 3: The procedure was the same as ethyl 1-methyl-4-[4-(1-methyl-4-[1-methyl-4-(3-[1-methyl-4-(1-methylimidazole-2-amido)pyrrol-2-yl]formamido}propanamido)imidazole-2-amido]pyrrol-2-yl}formamido)butanamido]imidazole-2-carboxylate (Example 2 step 9). 4-[2R)-2-[tert-butoxycarbonyl)amino]-4-[(-methyl-4-{1-methyl-4-[1-methyl-4-(1-methylpyrrole-2-amido)pyrrole-2-amido]imidazole-2-amido}pyrrol-2-yl)formamnido]butanamido]-1-methylimidazole-2-carboxylic acid (370.00 mg) was used, and 500.00 mg of the desired product was obtained as a red solid (95.02% yield). LC/MS: mass calcd. For C₅₃H₆₅N₁₉O₁₂: 1159.51, found: 1161.05 [M+H]⁺.

Step 4: The procedure was the same as 4-[3-[(tert-butoxycarbonyl)amino]propanamido]-1-methylimidazole-2-carboxylic acid (Example 1 step 3), but the reaction time was 1.0 h. Ethyl 4-{3-[(4-{4-[(2R)-2-[(tert-butoxycarbonyl) amino]-4-[(1-methyl-4-{1-methyl-4-[1-methyl-4-(1-methylpyrrole-2-amido)pyrrole-2-amido]imidazole-2-amido}pyrrol-2-yl)formamido]butanamido]-1-methylimidazole-2-amido}-1-methylpyrrol-2-yl)formamido]propanamido}-1-methylimidazole-2-carboxylate (300.00 mg) was used, and 240.00 mg of desired product was obtained as a yellow solid (81.98% yield). LC/MS: mass calcd. For C₅₁H₆₁N₁₉O₁₂: 1131.47, found: 1133.05 [M+H]⁺.

### Example 8. Synthesis of 4-{4-[(2S)-2-[(tert-butoxycarbonyl)amino]-4-{[1-methyl-4-(3-{[1-methyl-4-(1-methylimidazole-2-amido)pyrrol-2-yl]formamido}propanamido)imidazol-2-yl]formamido}butanamido]-1-methylpyrrole-2-amidol-1-methylimidazole-2-carboxylic acid (PA-023)

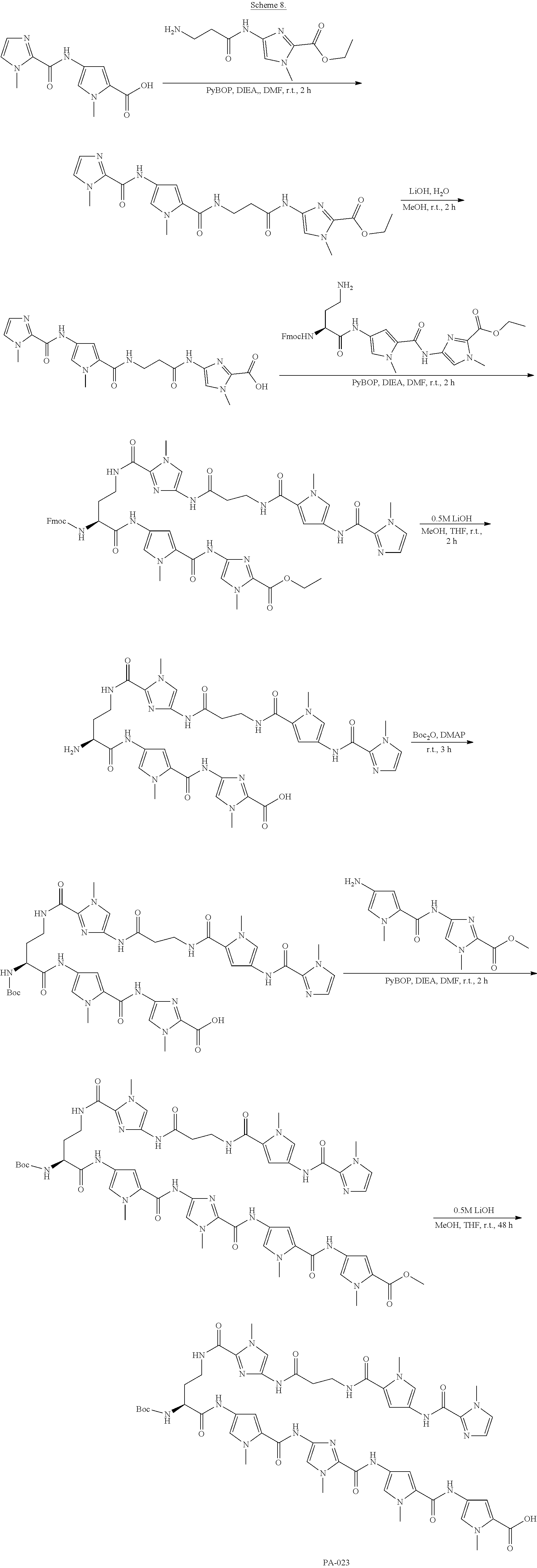

Step 1: The procedure was the same as ethyl 1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylate (Example 2 step 9), but the reaction time was 2.0 h. Ethyl 4-(3-aminopropanamido)-1-methylimidazole-2-carboxylate (1.50 g) was used, and 2.00 g of the desired product was obtained as an off-white solid (68.09% yield). LC/MS: mass calcd. For C₂₁H₂₆N₈O₅: 470.20, found: 471.40 [M+H]⁺.

Step 2: The procedure was the same as 4-[3-[(tert-butoxycarbonyl)amino]propanamido]-1-methylimidazole-2-carboxylic acid (Example 1 step 3), but the reaction temperature was room temperature and the reaction time was 2.0 h. Ethyl 1-methyl-4-(3-{[1-methyl-4-(1-methylimidazole-2-amido)pyrrol-2-yl]formamido}propanamido)imidazole-2-carboxylate (2.00 g) was used, and 1.80 g of 1-methyl-4-(3-{[1-methyl-4-(1-methylimidazole-2-amido)pyrrol-2-yl]formamido}propanamido)imidazole-2-carboxylic acid was obtained as an off-white solid (95.71% yield). LC/MS: mass calcd. For C₁₉H₂₂N₈O₅: 442.17, found: 443.10 [M+H]⁺.

Step 3: The procedure was the same as ethyl 1-methyl-4-[4-({1-methyl-4-[1-methyl-4-(3-{[1-methyl-4-(1-methylimidazole-2-amido)pyrrol-2-yl|formamido}propanamido)imidazole-2-amido pyrrol-2-yl}formamido)butanamido]imidazole-2-carboxylate (Example 2 step 9), but the reaction time was 2.0 h. Ethyl 4-{4-[(2S)-4-amino-2-{[(9H-fluoren-9-ylmethoxy)carbonyl]amino}butanamido]-1-methylpyrrole-2-amido}-1-methylimidazole-2-carboxylate (1.60 g) was used, and 1.90 g of the desired product was obtained as a yellow solid (70.20% yield). LC/MS: mass calcd. For C₅₁H₅₅N₁₅O₁₀: 1037.43, found: 1038.45 [M+H]⁺.

Step 4: A mixture of ethyl 4-{4-[(2S)-2-{[(9H-fluoren-9-ylmethoxy)carbonyl]amino}-4-{[1-methyl-4-(3-{[1-methyl-4-(1-methylimidazole-2-amido)pyrrol-2-yl]formamido}propanamido)imidazol-2-yl]formamido}butanamido]-1-methylpyrrole-2-amido}-1-methylimidazole-2-carboxylate (1.90 g, 1.83 mmol, 1.00 equiv) and LiOH (0.22 g, 9.15 mmol, 5.00 equiv) in MeOH (5.00 mL), THF (15.00 mL), and H₂O (18.30 mL) was stirred for 2.0 h at room temperature. The resulting mixture was used in the next step without further purification. LC/MS: mass calcd. For C₃₄H₄₁N₁₅O₈: 787.33, found: 788.40 [M+H]⁺.

Step 5: The mixture of 4-{4-[(2S)-2-amino-4-{[1-methyl-4-(3-{[1-methyl-4-(1-methylimidazole-2-amido)pyrrol-2-yl]formamido}propanamido)imidazol-2-yl]formamido}butananido]-1-methylpyrrole-2-amido}-1-methylimidazole-2-carboxylic acid (1.40 g, 1.78 mmol, 1.00 equiv) in MeOH/THF/H₂O (5.00 mL/15.00 mL/18.30 mL) was added di-tert-butyl dicarbonate (0.78 g, 3.55 mmol, 2.00 equiv) and DMAP (0.02 g, 0.18 mmol, 0.10 equiv). The reaction was stirred at room temperature for 3.0 h and then H₂O (30 mL) was added. The resulting mixture was filtered through a Celite pad and the solid was washed with EA (3×30 mL) to afford 4-{4-[(2S)-2-[(tert-butoxycarbonyl)amino]-4-{[1-methyl-4-(3-{[1-methyl-4-(1-methylimidazole-2-amido)pyrrol-2-yl]formamido}propanamido)imidazol-2-yl]formamido}butanamido]-1-methylpyrrole-2-amido}-1-methylimidazole-2-carboxylic acid (1.20 g, 76.05% yield) as a yellow solid. LC/MS: mass calcd. For C₃₉H₄₉N₁₅O₁₀: 887.38, found: 888.45 [M+H]⁺.

Step 6: The procedure was the same as 1-methyl-4-[4-({1-methyl-4-[1-methyl-4-(3-{[1-methyl-4-(1-methylimidazole-2-amido)pyrrol-2-yl]formamido}propanamido) imidazole-2-amido]pyrrol-2-yl}formamido)butanamido]imidazole-2-carboxylate (Example 2 step 9), but the reaction time was 2.0 h. 4-{4-[(2S)-2-[(tert-butoxycarbonyl)amino]-4-{[1-methyl-4-(3-{[1-methyl-4-(1-methylimidazole-2-amido)pyrrol-2-yl]formamido}propanamido)imidazol-2-yl]formamido}butanamido]-1-methylpyrrole-2-amido}-1-methylimidazole-2-carboxylic acid (1.20 g) was used, and 1.10 g of the desired product was obtained as a yellow solid (71.01% yield). LC/MS: mass calcd. For C₅₂H₆₃N₁₉O₁₂: 1145.49, found: 1146.50 [M+H]⁺.

Step 7: The procedure was the same as 4-[4-(4-{4-[(2S)-2-[(tert-butoxycarbonyl)amino]-4-[(1-methyl-4-{1-methyl-4-[1-methyl-4-(1-methylimidazole-2-amido)pyrrole-2-amido]pyrrole-2-amido}imidazol-2-yl)formamido]butanamido]-1-methylpyrrole-2-amido}-1-methylimidazole-2-amido)-1-methylpyrrole-2-amido]-1-methylpyrrole-2-carboxylic acid. Methyl 4-[4-(4-{4-[(2S)-2-[(tert-butoxy carbonyl)amino]-4-{[1-methyl-4-(3-{[1-methyl-4-(1-methylimidazole-2-amido)pyrrol-2-yl]formamido}propanamido)imidazol-2-yl]formamido}butanamido]-1-methylpyrrole-2-amido}-1-methylimidazole-2-amido)-1-methylpyrrole-2-amido]-1-methylpyrrole-2-carboxylate (1.00 g) was used, and 400.00 mg of the desired product was obtained as a white solid (39.16% yield). LC/MS: mass calcd. For C₁H₆₁N₁₉O₁₂: 1131.47, found: 1132.65 [M+H]⁺

### Synthesis of Representative Compounds of the Disclosure

### Example 9. Synthesis of Compound B-1

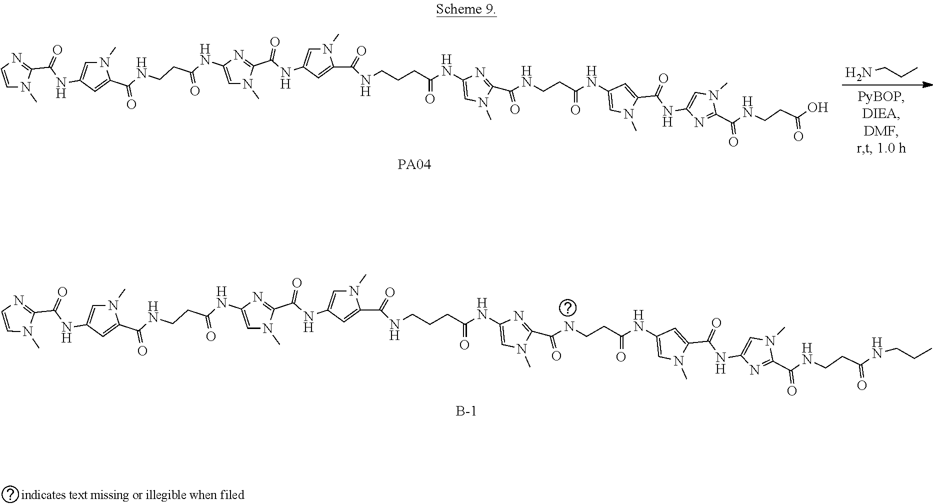

A solution of 3-(1-methyl-4-(1-methyl-4-(3-(1-methyl-4-(4-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)butanamido)-1H-imidazole-2-carboxamido)propanamido)-1H-pyrrole-2-carboxamido)-1H-imidazole-2-carboxamido)propanoic acid, intermediate polyamide (PA-04) (1.00 equiv), in DMF (3.00 mg) was added propan-1-amine (1.05 equiv), PyBOP (1.20 equiv), and DIEA (2.94 equiv); and the resulting mixture was stirred for 1.0 h at room temperature. The reaction mixture was purified by Prep-HPLC to afford 49.00 mg of the desired product as a white solid (25.17% yield). HRMS: mass calcd. for C₅₄H₆₈N₂O₁₁: 1200.5438, found: 1201.5473 [M+H]⁺.

### Example 10. Synthesis of Compound B-3

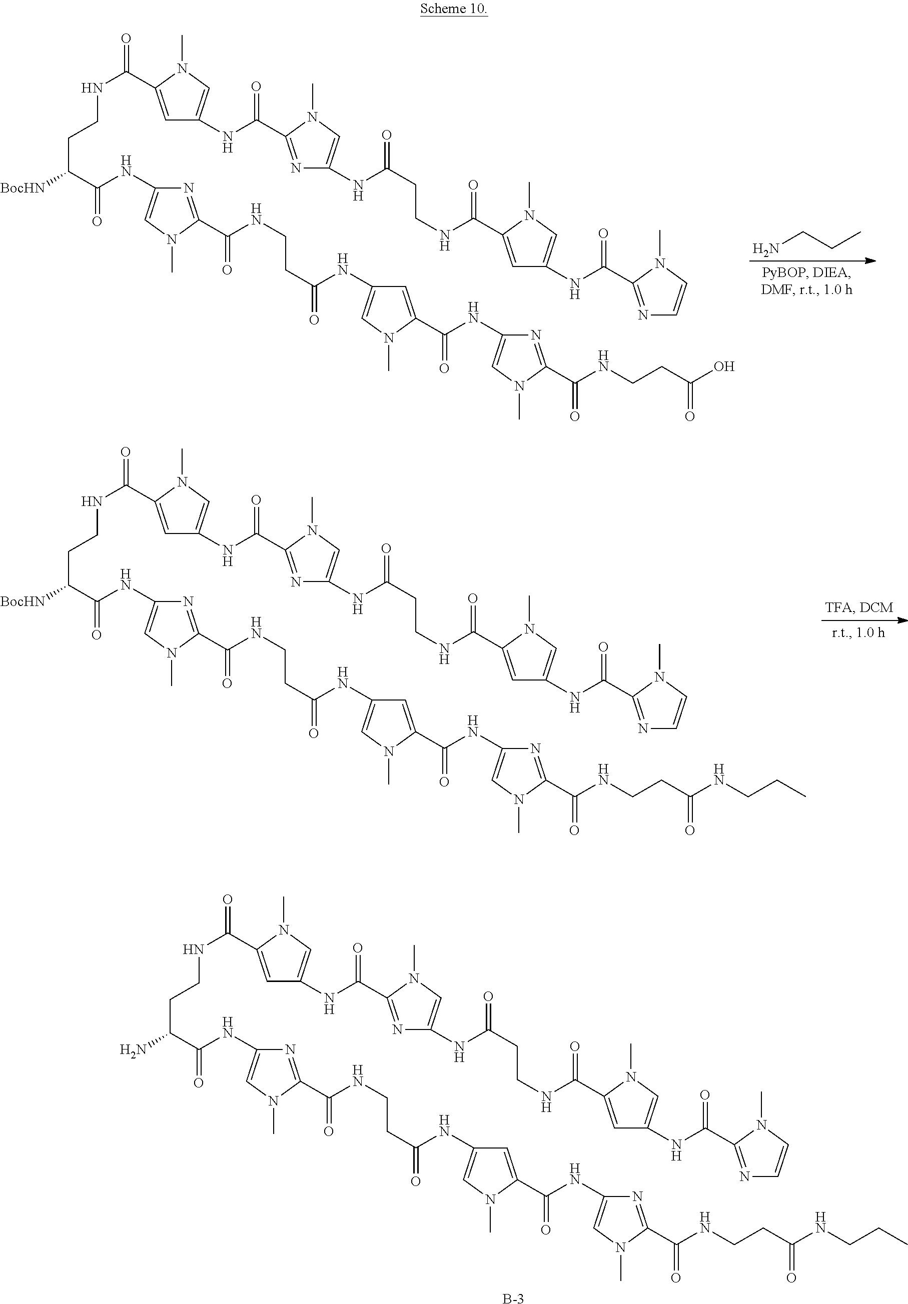

Step 1: The procedure was the same as Example 9 (Compound B-1). 3-[(4-{4-[3-({4-[(2R)-2-[(tert-butoxycarbonyl)amino]-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-1-methylimidazol-2-yl}formamido)propanamido]-1-methylpyrrole-2-amido}-1-methylimidazol-2-yl)formamido]propanoic acid (20.00 mg) was used to afford 20.00 mg of the desired product as white solid (96.88% yield). LC/MS: mass calcd. For C₅₉H₁₇N₂₃O₁₃: 1315.61, found: 1316.70 [M+H]+.

Step 2: The procedure was the same as Example 2 step 16, but the crude product was purified by Prep-HPLC. Tert-butyl N-[(1R)-1-[(1-methyl-2-{[2-({1-methyl-5-[(1-methyl-2-{[2-(propylcarbamoyl)ethyl]carbamoyl}imidazol-4-yl)carbamoyl]pyrrol-3-yl}carbamoyl)ethyl]carbamoyl}imidazole-4-yl)carbamoyl]-3-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)propyl]carbamate (20.00 g) was used, and 6.10 mg of the desired product was obtained as a white solid (32.53% yield). HRMS: mass calcd. For C₅₄H₆₉N₂₃O₁₁: 1215.5547, found: 1216.5581 [M+H]⁺

### Example 11. Synthesis of Compound B-79

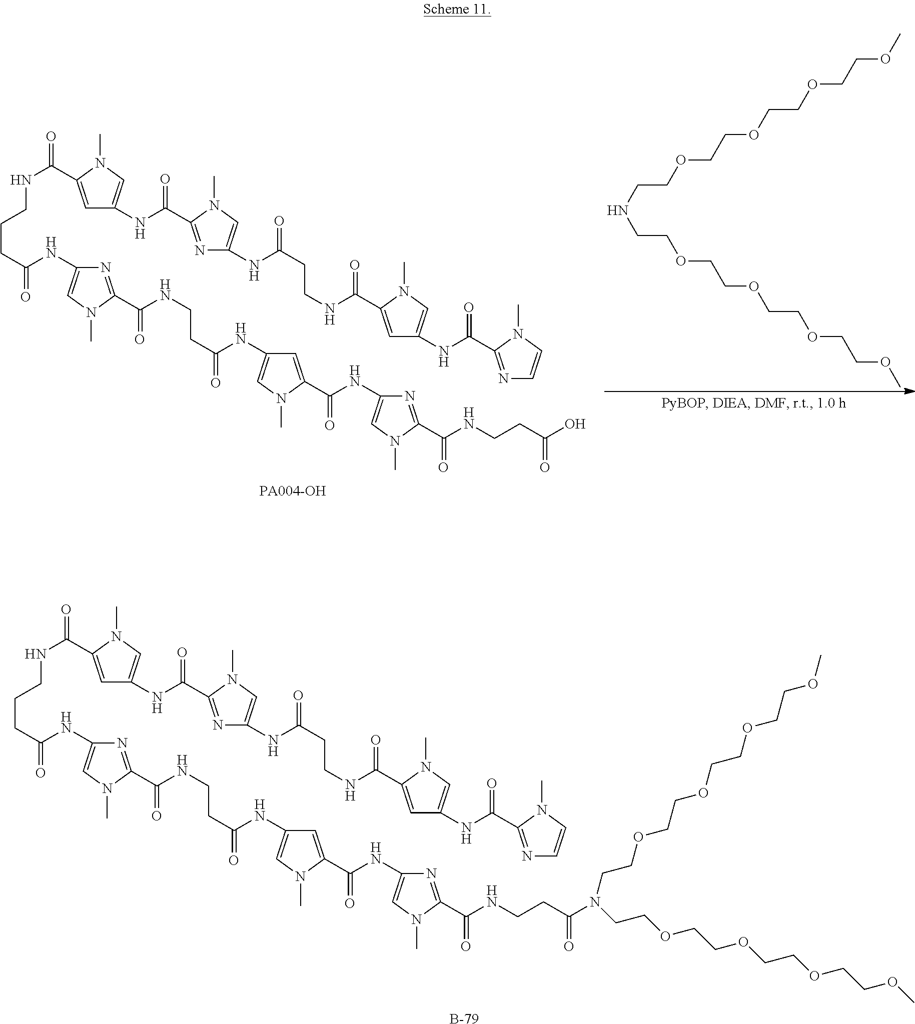

The synthesis is the same as Example 9 (Compound B-1). 3-[(1-Methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanoic acid (120.00 mg) was used to afford 60.60 mg of the desired product as a white solid (37.83% yield).

### Example 12. Synthesis of Compounds B-29 and B-156

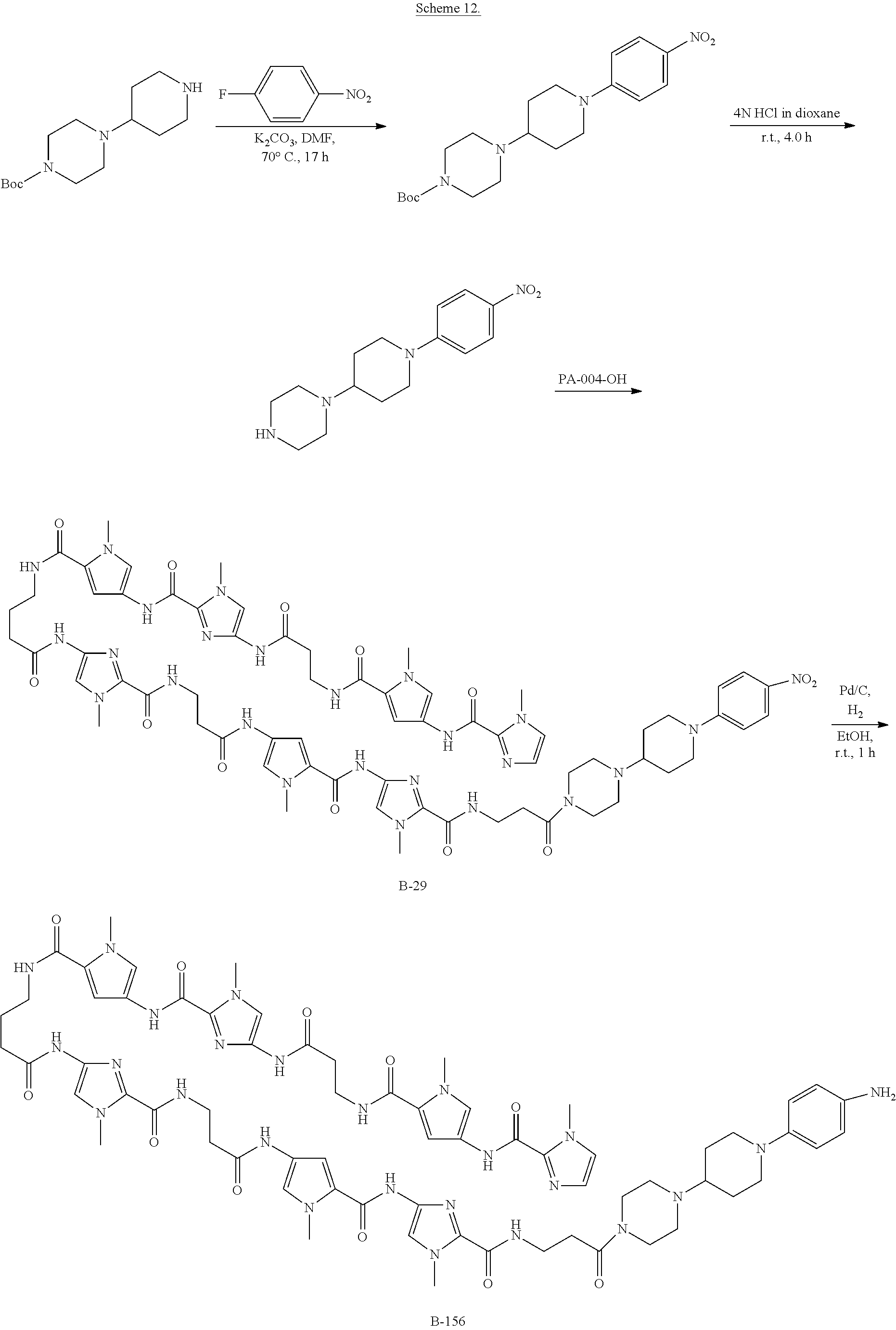

Step 1: Into a 250 ml flask was added tert-butyl 4-(piperidin-4-yl)piperazine-1-carboxylate (4.00 g, 14.85 mmol, 1.00 equiv), DMF (80 mL), tert-butyl 4-(piperidin-4-yl)piperazine-1-carboxylate (4.00 g, 14.85 mmol, 1.00 equiv), and K₂CO₃ (10.26 g, 74.24 mmol, 5.00 equiv), and the reaction was stirred at 80° C. for 17.0 h. The reaction was quenched with ice water (300 mL) and the precipitated solids were collected by filtration. washed with water (3×50 mL) and dried under vacuum. This resulted in tert-butyl 4-[1-(4-nitrophenyl)piperidin-4-yl]piperazine-1-carboxylate (4.7 g, crude) as a yellow solid. LC/MS: mass calcd. For C₂₀H₃₀N₄O₄: 390.23, found: 391.20[M+H]⁺.

Step 2: The procedure was the same as Example 2, but the reaction time was 4.0 h. Tert-butyl 4-[1-(4-nitrophenyl)piperidin-4-yl]piperazine-1-carboxylate (1.00 g) was used to afford 0.70 g of the desired product as a yellow oil (94.14%) yield. LC/MS: mass calcd. For C₁₈H₂₂N₄O₂: 290.17, found: 291.15 [M+H]⁺.

Step 3: Synthesis of Compound B-29. The procedure was the same as Example 9, but the reaction time was 2.0 h, and the reaction mixture was purified by Prep-HPLC. 1-[1-(4-Nitrophenyl)piperidin-4-yl]piperazine (30.03 mg) was used and 20.10 mg of the desired product was obtained as a yellow solid (19.67% yield). HRMS: mass calcd. For C₆₆H₅₁N₂₅O₁₃: 1431.6446, found: 1432.6538 [M+H]⁺.

Step 4: Synthesis of Compound 156. The procedure was the same as ethyl 4-amino-1-methylimidazole-2-carboxylate (Example 1). 1-Methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}-N-(3-{4-[1-(4-nitrophenyl)piperidin-4-yl]piperazin-1-yl}-3-oxopropyl)imidazole-2-carboxamide (17.00 mg) was used to afford 7.90 mg of the desired product as an off-white solid (47.34% yield). HRMS: mass calcd. For C₆₆H₈₃N₂₅O₁₁: 1401.6704, found: 1402.6823 [M+H]⁺.

### Example 13. Synthesis of Compound B-367

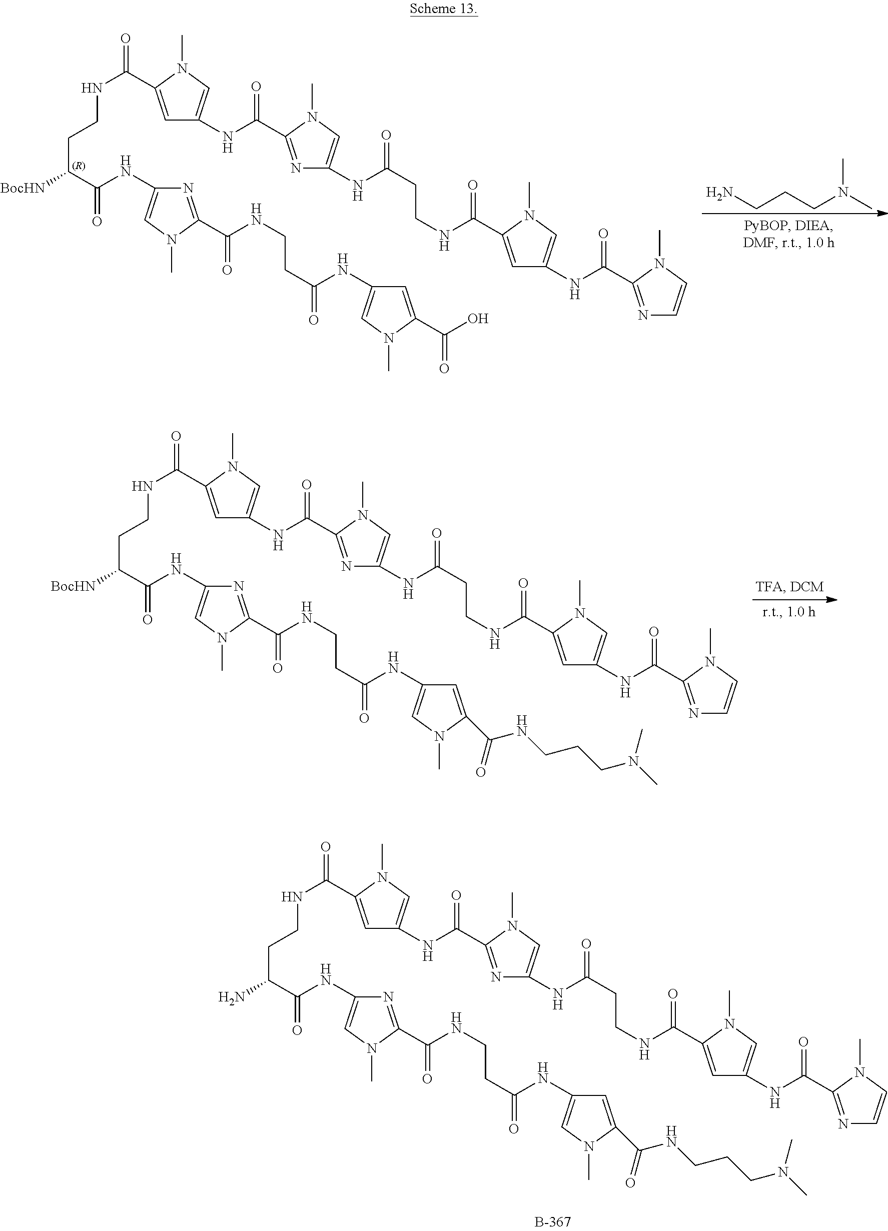

The procedure was the same as N-(5-{[(3R)-3-amino-3-[(1-methyl-2-{[2-({1-methyl-1-[(1-methyl-2-{[2-(propylcarbamoyl)ethyl]carbamoyl}imidazol-4-yl)carbamoyl]pyrrol-3-yl}carbamoyl)ethyl]carbamoyl}imidazol-4-yl)carbamoyl]propyl]carbamoyl}-1-methylpyrrol-3-yl)-1-methyl-4-(3-{[1-methyl-4-(1-methylimidazole-2-amido)pyrrol-2-yl]formamido}propanamido)imidazole-2-carboxamide (Example 10). HRMS: mass calcd. C₄₈H₆₄N₂₀O₉: 1064.5165, found: 1065.5226 [M+H]⁺.

### Example 14. Synthesis of Compound B1-127

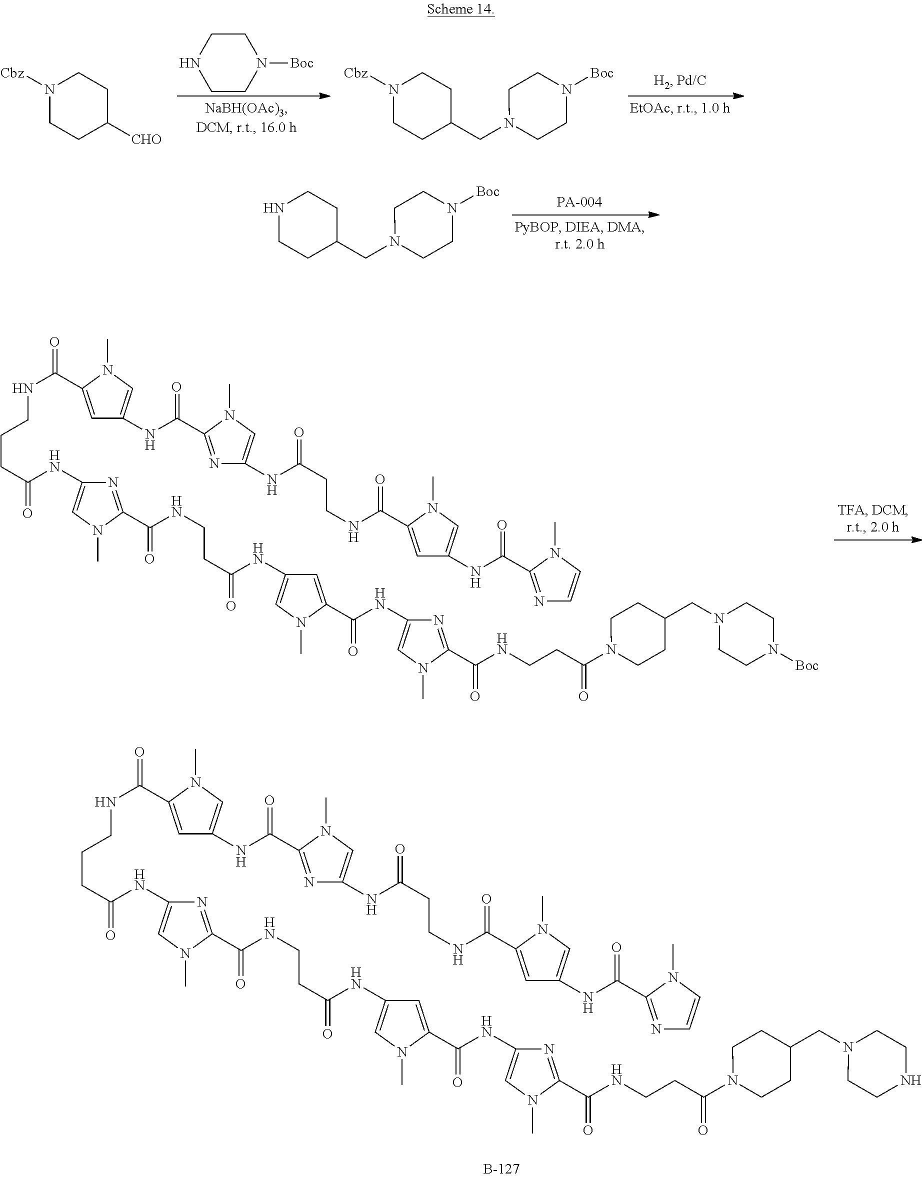 [SMILES: O=CC1CCN(C(=O)OCc2ccccc2)CC1 ; CC(C)(C)OC(=O)N1CCNCC1 ; CC(C)(C)OC(=O)N1CCN(CC2CCN(C(=O)OCc3ccccc3)CC2)CC1 ; CC(C)(C)OC(=O)N1CCN(CC2CCNCC2)CC1 ; Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)N4CCC(CN5CCN(C(=O)OC(C)(C)C)CC5)CC4)n3)n(C)c2)n1 ; Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)N4CCC(CN5CCNCC5)CC4)n3)n(C)c2)n1]

Step 1: A mixture of benzyl 4-formylpiperidine-1-carboxylate (2.00 g, 8.09 mmol, 1.00 equiv) and tert-butyl piperazine-1-carboxylate (1.51 g, 8.08 mmol, 1.00 equiv) in DCM (20 mL) was stirred at room temperature for 30 min. Then NaBH(OAc)₃ (1.71 g, 8.08 mmol, 1.00 equiv) was added and the resulting mixture was stirred at room temperature for 16.0 h. Next the mixture was washed with 2×30 mL of aq. NaOH (2 M), 2×30 mL of aq. HCl (2 M), and 2×30 mL of brine. The washed mixture was dried over Na₂SO₄, filtered, and concentrated under reduced pressure to afford 2.70 g of the desired product as a yellow solid (79.95% yield). LC/MS: mass calcd. For C₂₃H₃₅N₃O₄: 417.26, found: 418.15 [M+H]⁺.

Step 2: The procedure was the same as Example 1, but the reaction time was 16.0 h. Tert-butyl 4-({1-[(benzyloxy)carbonyl]piperidin-4-yl}methyl)piperazine-1-carboxylate (3.00 g) was used to afford 1.40 g of the desired product as a yellow solid (68.75% yield). LC/MS: mass calcd. For C₁₅H₂₉N₃O₂: 283.23, found: 284.15 [M+H]⁺.

Step 3: The procedure was the same as Example 9 (Compound B-1), but the reaction time was 2.0 h. Tert-butyl 4-(piperidin-4-ylmethyl)piperazine-1-carboxylate (120.00 mg) was used to afford 270.00 mg of the desired product as a yellow solid (44.73% yield). LC/MS: mass calcd. LC/MS: mass calcd. For C₆₆H₈₈N₂₄O₁₃: 1424.70, found: 1425.50 [M+H]⁺.

Step 4: The procedure was the same as Example 2 step 12, but the reaction time was 2.0 h and the reaction mixture was purified by Prep-HPLC. Tert-butyl 4-[(1-{3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanoyl}piperidin-4-yl)methyl]piperazine-1-carboxylate (250.00 mg) was used to afford 5.70 mg of the desired product as a light yellow solid (2.45% yield). LC/MS: mass calcd. HRMS: mass calcd. For C₆₁H₈₀N₂₄O₁₁: 1324.64, found: 1325.65 [M+H]⁺.

### Example 15. Synthesis of Compounds B-126, B-378, and B-379

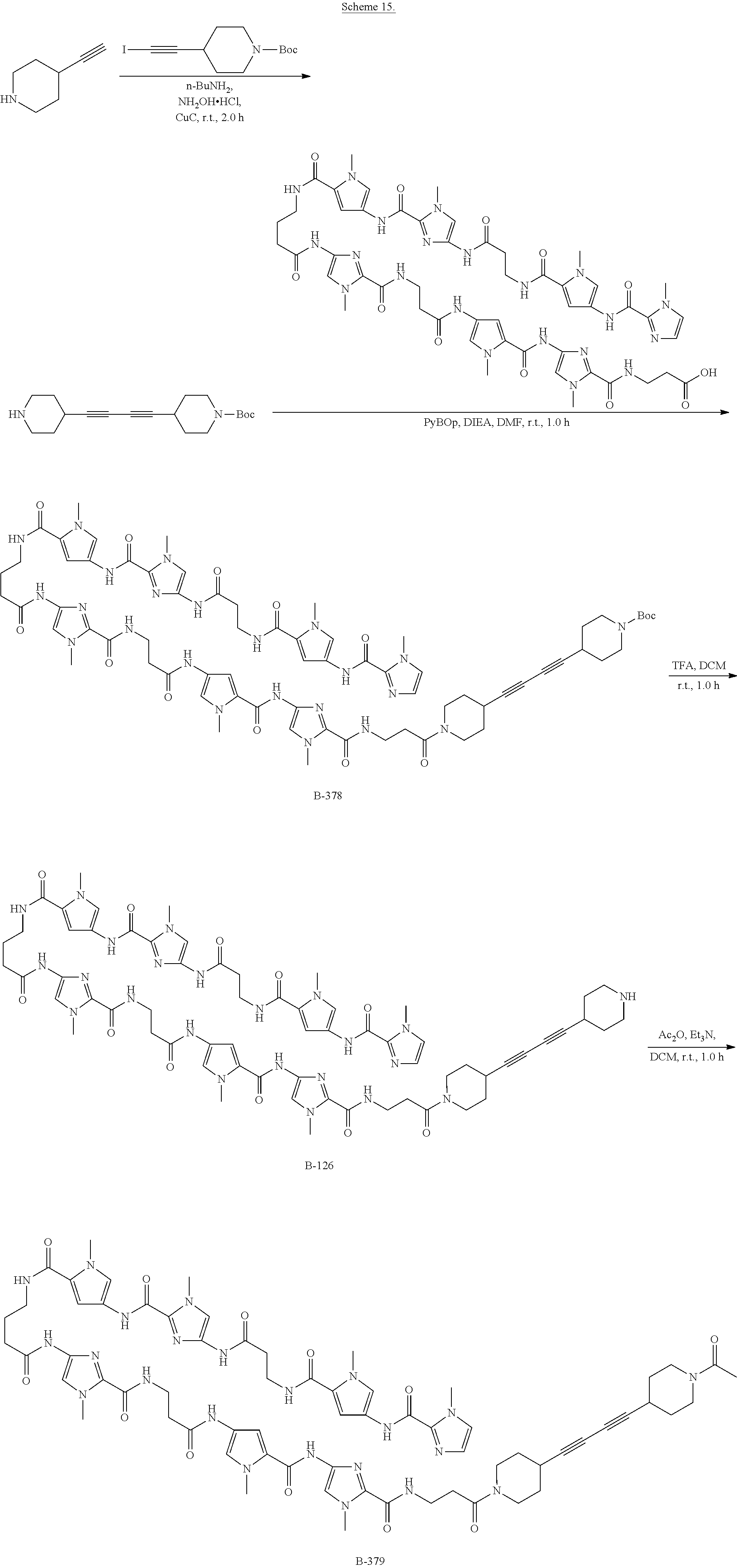

Step 1: To a stirred solution of CuCl (9.07 mg, 0.09 mmol, 0.20 equiv) in BuNH₂ (0.50 mL) and H₂O (1.50 mL) was added NH₂OH·HCl (63.65 mg, 0.912 mmol, 2.00 equiv). The mixture was stirred for 15 min at room temperature, and then tert-butyl 4-(2-iodoethynyl)piperidine-1-carboxylate (153.51 mg, 0.46 mmol, 1.00 equiv) and 4-ethynylpiperidine (50.00 mg, 0.46 mmol, 1.00 equiv) in BuNH₂ (0.50 mL) were added at 0° C. The resulting mixture was stirred for 2.0 h at room temperature. Then the reaction mixture was diluted with H₂O (5.00 mL) and extracted with EA (3×2.00 mL). The combined organic layers were washed with NaCl (2.00 mL), dried over Na₂SO₄, filtered, and concentrated. The residue was purified using silica gel column chromatography and eluted with DCM/MeOH (15:1) to afford tert-butyl 4-[4-(piperidin-4-yl)buta-1,3-diyn-1-yl]piperidine-1-carboxylate (122.00 mg, 74.08%) as a brown solid. LC/MS: mass calcd. For C₁₉H₂₈N₂O₂: 316.22, found: 317.10 [M+H]⁺.

Step 2: Synthesis of Compound B-378. The procedure was the same as Example 9, (Compound B-1). After the reaction, the mixture was poured into ice-water and the obtained solid were used directly in the next step without further purification. 3-[(1-Methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanoic acid (100.00 mg) was used to afford 90.00 mg of the desired product as a white solid (50.58% yield). HRMS: mass calcd. C₆₈H₈₆N₂₄O₁₂: 1457.6854, found: 1458.6929 [M+H]⁺.

Step 3: Synthesis of Compound B-126. The procedure was the same as Example 2 step 16, but the crude was purified by Prep-HPLC. Tert-butyl 4-[4-(1-{3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanoyl}piperidin-4-yl)buta-1.3-diyn-1-yl]piperidine-1-carboxylate (80.00 mg) was used to afford 25.20 mg of the desired product as a yellow solid. HRMS: mass calcd. For C₆₅H₁₉N₂₃O₁₁: 1357.6329, found: 1358.6396 [M+H]⁺.

Step 4: Synthesis of Compound B-379. The procedure was the same as Example 5 step 2, and the obtained solid was purified by Prep-HPLC. 1-Methyl-4-(3-{[1-methyl-4-(1-methylimidazole-2-amido)pyrrol-2-yl]formamido}propanamido)-N-(1-methyl-5-{[3-({1-methyl-2-[(2-{[1-methyl-5-({1-methyl-2-[(3-oxo-3-{4-[4-(piperidin-4-yl)buta-1,3-diyn-1-yl]piperidin-1-yl}propyl)carbamoyl]imidazol-4-yl}carbamoyl)pyrrol-3-yl]carbamoyl}ethyl)carbamoyl]imidazol-4-yl}carbamoyl)propyl]carbamoyl}pyrrol-3-yl)imidazole-2-carboxamide (112.00 mg) was used to afford 18.80 mg of desired product as a white solid (16.19% yield). HRMS: mass calcd. For C₆₇H₈₁N₂₃O₁₂: 1399.6435, found: 1400.6460 [M+H]⁺.

### Example 16. Synthesis of Compound B-137

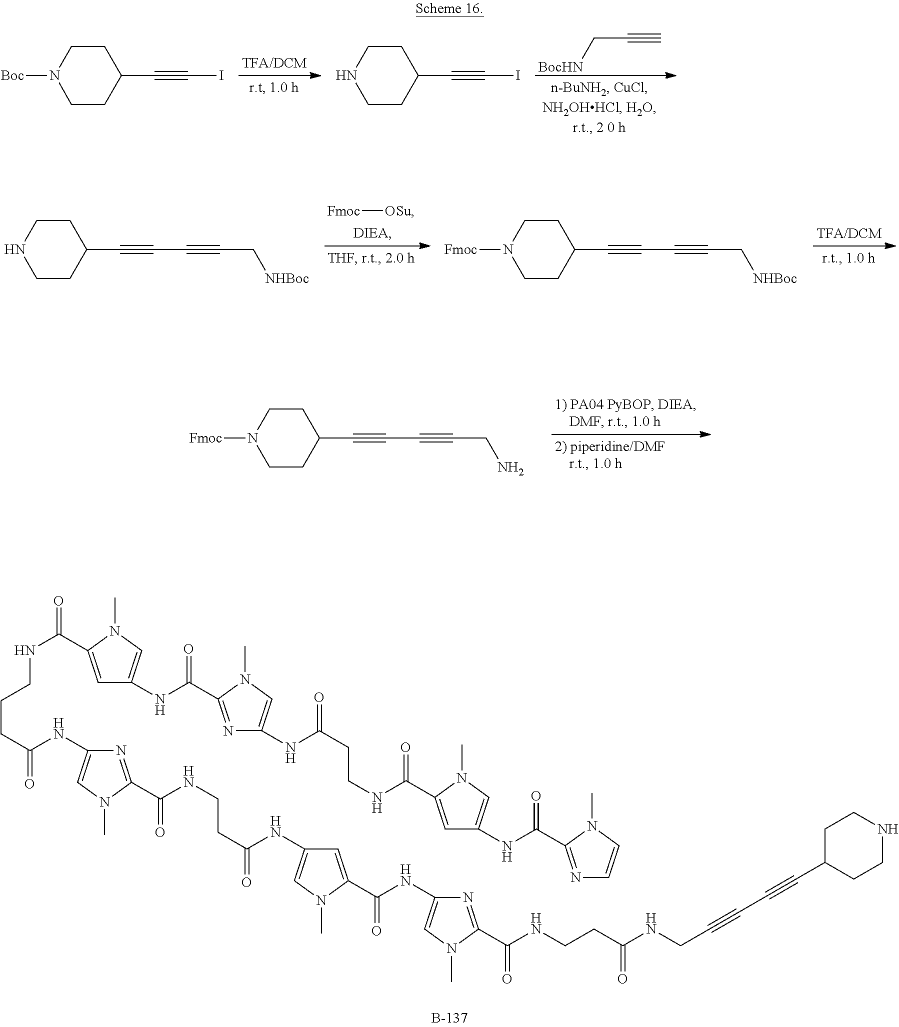

Step 1: The procedure was the same as Example 2 step 16. Tert-butyl 4-(2-iodoethynyl)piperidine-1-carboxylate (2.80 g) was used to afford 2.80 g of crude product as a yellow solid. LC/MS: mass calcd. For C₇H₁₀IN: 234.99, found: 235.95 [M+H]⁺.

Step 2: The procedure was the same as tert-butyl 4-[4-(piperidin-4-yl)buta-1,3-diyn-1-yl]piperidine-1-carboxylate (Example 15 step 1), but the crude product was used directly in the next step. 4-(2-Iodoethynyl)piperidine (500.00 mg) was used to afford 400.00 mg of desired product as a yellow solid. LCMS: mass calcd. For C₁₅H₂₂N₂O₂: 262.17, found: 263.30[M+H]⁺.

Step 3: A solution of tert-butyl N-[5-(piperidin-4-yl)penta-2,4-diyn-1-yl]carbamate (130.00 mg, 0.50 mmol, 1.00 equiv), 2,5-dioxopyrrolidin-1-yl-9H-fluoren-9-ylmethyl carbonate (180.00 mg, 0.53 mmol, 1.08 equiv) and DIEA (127.00 mg, 0.98 mmol, 1.98 equiv) in THF (3.00 mL) was stirred for 2.0 h at room temperature. The reaction was quenched with water (30 mL) at room temperature and was extracted with EA (3×30 mL). The combined organic layers were washed with water (2×20 mL) and dried over anhydrous Na₂SO₄. After filtration, the filtrate was concentrated under reduced pressure. The residue was purified by Prep-TLC (PE/EA 3:1) to afford 9H-fluoren-9-ylmethyl 4-{5-[(tert-butoxycarbonyl)amino]penta-1,3-diyn-1-yl}piperidine-1-carboxylate (110.00 mg, 45.81% yield) as a white oil. LCMS: mass calcd. For C₃₀H₃₂N₂O₄: 484.24, found: 485.25[M+H]⁺.

Step 4: The procedure was the same as Example 2 step 16. 9H-Fluoren-9-ylmethyl 4-{5-[(tert-butoxycarbonyl)amino]penta-1,3-diyn-1-yl}piperidine-1-carboxylate (110.00 mg) was used to afford 110.00 mg of the desired product as a yellow oil. LCMS: mass calcd. For C₂₅H₂₄N₂O₂: 384.18, found: 385.25[M+H]⁺.

Step 5: A solution of 3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanoic acid (270.00 mg, 0.23 mmol, 0.81 equiv) and 9H-fluoren-9-ylmethyl 4-(5-aminopenta-1,3-diyn-1-yl)piperidine-1-carboxylate (110.00 mg, 0.29 mmol, 1.00 equiv), PyBOP (150.00 mg, 0.29 mmol, 1.01 equiv), and DIEA (160.00 mg, 1.24 mmol, 4.33 equiv) in DMF (3.00 mL) was stirred for 1.0 h at room temperature. Then piperidine (1.0 mL) was added and the reaction mixture was stirred for another 1.0 h at room temperature. The reaction was purified by reverse flash chromatography with the following conditions: Column, C18 silica gel; Mobile Phase, ACN in water (0.05% TFA), 5% to 48% gradient in 25 min; Detector, UV 254 nm. The fractions were combined and concentrated to afford the desired product (120.00 mg, 70.22% yield) as a yellow solid.

The crude product (70 mg) was purified by Prep-HPLC with the following conditions. Column: XBridge Shield RP18 OBD Column, 19*250 mm, 10 μm; Mobile Phase A: water (10 mmol/L NH₄HCO₃), Mobile Phase B: ACN; Flow rate: 25 mL/min; Gradient: 30% B to 44% B in 10 min, 44% B; Wave Length: 254 nm: RT1 (min): 9.72. The fractions were combined and lyophilized to afford 1-methyl-4-(3-{[1-methyl-4-(1-methylimidazole-2-amido)pyrrol-2-yl]formamido}propanamido)-N-(1-methyl-5-{[3-({1-methyl-2-[(2-{[1-methyl-5-({1-methyl-2-[(2-{[5-(piperidin-4-yl)penta-2,4-diyn-1-yl]carbamoyl}ethyl)carbamoyl]imidazol-4-yl}carbamoyl)pyrrol-3-yl]carbamoyl}ethyl)carbamoyl]imidazol-4-yl}carbamoyl)propyl]carbamoyl}pyrrol-3-yl)imidazole-2-carboxamide (12.60 mg, 18.00 yield) as a white solid. HRMS (ESI): mass calcd. For C₆₁H₇₃N₂₃O₁₁: 1303.5860, found: 1304.5942 [M+H]⁺.

### Example 17. Synthesis of Compound B-150

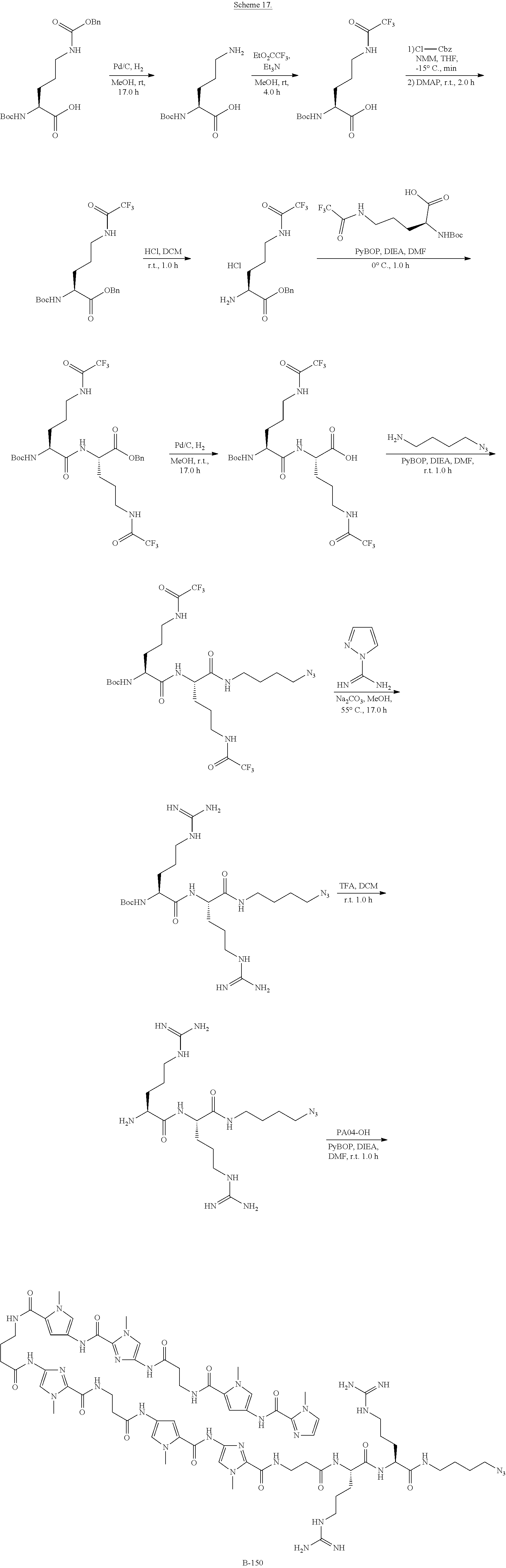

Step 1: To a stirred solution of (2S)-5-amino-2-[(tert-butoxycarbonyl)amino]pentanoic acid (6.30 g, 27.12 mmol, 1.00 equiv) in MeOH (100.00 mL) was added ethyl 2,2,2-trifluoroacetate (5.78 g, 40.68 mmol, 1.50 equiv) and Et₃N (5.49 g, 54.24 mmol, 2.00 equiv) at room temperature. The resulting mixture was stirred for 4.0 h at room temperature. Then Pd/C was filtered out and the resulting mixture was quenched with water (100 mL), extracted with EA (100 mL), and the water phase was adjust to PH=3-5 with 2M HCl, and then extracted with EA (3*100 ml). The organic phase was combined, washed with NaCl solution (100 mL), dried by Na₂SO₄ (filtered out), and concentrated. This resulted in (2S)-2-[(tert-butoxycarbonyl)amino]-5-(2,2,2-trifluoroacetamido)pentanoic acid (12.00 g crude) as an orange oil. LC/MS: mass calcd. For C₁₂H₁₉F₃N₂O₅:328.12, found: 351.10 [M+Na]⁺.

Step 2: To a stirred solution of (2S)-5-amino-2-[(tert-butoxycarbonyl)amino]pentanoic acid (6.30 g, 27.12 mmol, 1.00 equiv) in MeOH (100.00 mL) was added ethyl 2,2,2-trifluoroacetate (5.78 g, 40.68 mmol, 1.50 equiv) and Et₃N (5.49 g, 54.24 mmol, 2.00 equiv) at room temperature. The resulting mixture was stirred for 4.0 h at room temperature. Then Pd/C was filtered out, and the resulting mixture was quenched with water (100 mL), extracted with EA (100 mL), the water phase was adjust to pH=3-5 by 2M HCl, then extracted with EA (3×100 ml), the organic phase was combined and washed with NaCl solution (100 mL), dried by Na₂SO₄ (filtered out), the organic phase was concentrated. This resulted in (2S)-2-[(tert-butoxycarbonyl)amino]-5-(2,2,2-trifluoroacetamido)pentanoic acid (12.00 g crude) as an orange oil.

Step 3: To a stirred solution of (2S)-2-{[(2,2-dimethylpropanoyl)oxy]amino}-5-(2,2,2-trifluoroacetamido)pentanoic acid (1.00 g, 3.05 mmol, 1.00 equiv) in THF (10.00 mL) was added NMM (308.11 mg, 3.05 mmol, 1.00 equiv) dropwise at room temperature. The result mixture was cooled to −15° C. and Cbz-Cl (545.62 mg, 3.20 mmol, 1.05 equiv) in THF (5.00 mL) was added to the result mixture at −15° C. The resulting mixture was stirred for 2 min at −15° C. and warmed to 0° C. and stirred for 15 min. DMAP (93.04 mg, 0.761 mmol, 0.25 equiv) was added to the result mixture and the result mixture was allowed to warm to room temperature and stirred for 2.0 h. The reaction was quenched with H₂O (15 mL) at 0° C. The resulting mixture was extracted with EA (3×15 mL). The combined organic layers were washed with brine (1×15 mL) and dried over anhydrous Na₂SO₄. After filtration, the filtrate was concentrated under reduced pressure to afford benzyl (2S)-2-{[(2,2-dimethylpropanoyl)oxy]amino}-5-(2,2,2-trifluoroacetamido)pentanoate (780.00 mg, 52.22%) as a yellow oil. LC/MS: mass calcd. For C₁₉H₂₅F₃N₂O₅: 418.17, found: 441.30[M+Na]⁺.

Step 4: The procedure was the same as Example 2, but the solvent used was CH₂Cl₂. Benzyl (2S)-2-[(tert-butoxycarbonyl)amino]-5-(2,2,2-trifluoroacetamido)pentanoate (700.00 mg) was used to afford 440.00 mg of the desired product as a brown oil. LC/MS: mass calcd. For C₁₄H₁₇F₃N₂O₃:318.12, found: 319.30 [M+H]⁺.

Step 5: The procedure was the same as Example 9. After the reaction, the reaction mixture was poured into ice-water and the solid was used in the next step without further purification. (2S)-2-[(Tert-butoxycarbonyl)amino]-5-(2,2,2-trifluoroacetamido)pentanoic acid (477.00 mg) was used to afford 550.00 mg of the desired product as a white solid (34.37% yield).

Step 6: To a solution of benzyl (2S)-2-[(2S)-2-[(tert-butoxycarbonyl)amino]-5-(2,2,2-trifluoroacetamido)pentanamido]-5-(2,2,2-trifluoroacetamido)pentanoate (500.00 mg, 1.00 equiv) in MeOH (10.00 mL) was added Pd/C (200 mg, 40% w/w). The reaction was stirred for 17.0 h at room temperature under H₂ atmosphere. Then the mixture was filtrated and the filtrate was concentrated to afford (2S)-2-[(2S)-2-[(tert-butoxycarbonyl)amino]-5-(2,2,2-trifluoroacetamido)pentanamido]-5-(2,2,2-trifluoroacetamido)pentanoic acid (390.00 mg crude) as a colorless oil. LC/MS: mass calcd. For C₁₉H₂₈F₆N₄O₇:538.19, found: 561.35 [M+Na]⁺.

Step 7: The procedure was the same as Example 9. After the reaction, the reaction mixture was poured into ice-water and the obtained solid was used without further purification. (2S)-2-[(2S)-2-[(tert-butoxycarbonyl)amino]-5-(2,2,2-trifluoroacetamido) pentanamido]-5-(2,2,2-trifluoroacetamido)pentanoic acid (390.00 mg) was used to afford 300.00 mg of the desired product as a white solid (65.27% yield). LC/MS: mass calcd. For C₂₃H₃₆F₆N₈O₆:634.27, found: 657.30 [M+Na]⁺.

Step 8: To a solution of tert-butyl N-[(1S)-1-{[(1S)-1-[(4-azidobutyl)carbamoyl]-4-(2,2,2-trifluoroacetamido)butyl]carbamoyl}-4-(2,2,2-trifluoroacetamido)butyl]carbamate (280.00 mg, 0.44 mmol, 1.00 equiv) in MeOH (5.00 mL) was added 2.5 mL Na₂CO₃ aqueous solution (467.66 mg, 4.41 mmol, 10.00 equiv). Then the reaction was stirred at 55° C. for 17.0 h. The reaction mixture was filtered and the filtrate was concentrated. The residue was purified by reverse flash chromatography with the following conditions: Column, C18 silica gel; Mobile Phase, ACN in water (0.05% NH₄HCO₃), 10% to 80% gradient in 40 min; detector, UV 254 nm. The fractions were combined and concentrated to afford tert-butyl N-[(1S)-1-{[(1S)-1-[(4-azidobutyl) carbamoyl]-4-carbamimidanidobutyl]carbamoyl}-4-carbamimidamidobutyl]carbamate (230.00 mg, 98.98% yield) as a colorless oil. LC/MS: mass calcd. For C₂₁H₄₂N₁₂O₄:526.34. found: 527.35 [M+H]⁺.

Step 9: The procedure was the same as Example 2 step 16. Tert-butyl N-(1S)-1-{|(1S)-1-|(4-azidobutyl) carbamoyl]-4-carbamimidamidobutyl]carbamoyl}-4-carbamimidamidobutyl]carbamate (60.00 mg) was used to afford 60.00 mg crude of the desired product as a brown-yellow oil. LC/MS: mass calcd. For C₁₆H₃₄N₂O₂:426.29, found: 427.50 [M+H]⁺.

### Example 18. Synthesis of Compound B-76

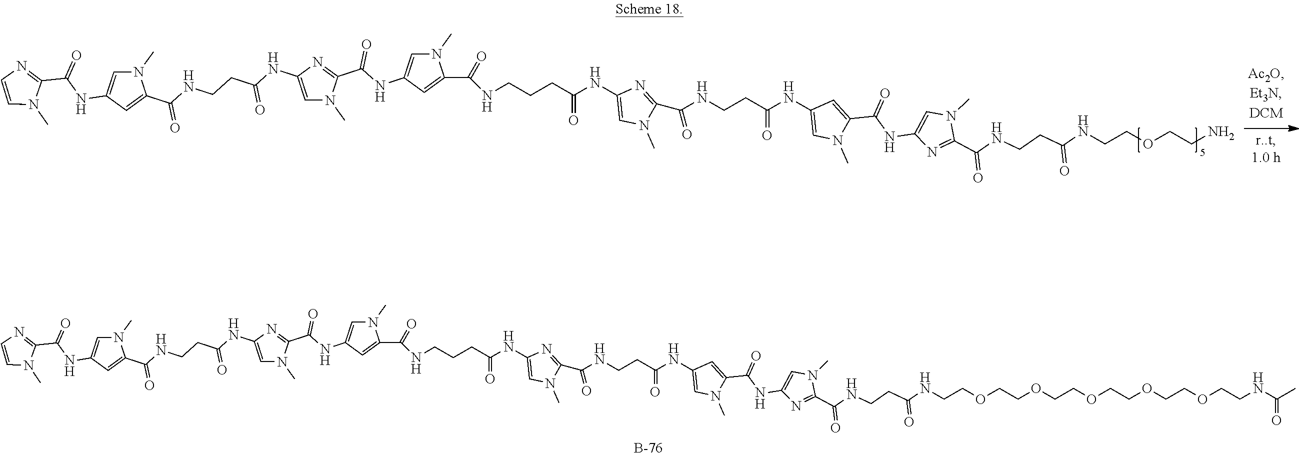

The procedure was the same as Example 5 step 2, and the obtained crude was purified by Prep-HPLC. N-{5-[(3-{[2-({2-[(5-{[2-({2-[(17-amino-3,6,9,12,15-pentaoxaheptadecan-1-yl)carbamoyl]ethyl}carbamoyl)-1-methylimidazol-4-yl]carbamoyl}-1-methylpyrrol-3-yl)carbamoyl]ethyl}carbamoyl)-1-methylimidazol-4-yl]carbamoyl}propyl)carbamoyl]-1-methylpyrrol-3-yl}-1-methyl-4-(3-{[1-methyl-4-(1-methylimidazole-2-amido)pyrrol-2-yl]formamido}propanamido) imidazole-2-carboxamide (500.00 mg) was used to afford 158.90 mg of the desired product as a white solid (30.30% yield). HRMS: mass calcd. For C₆₅H₈₉N₂₃O₁₇: 1463.6807, found: 1464.6862[M+H]⁺.

### Example 19. Synthesis of Compound B-296

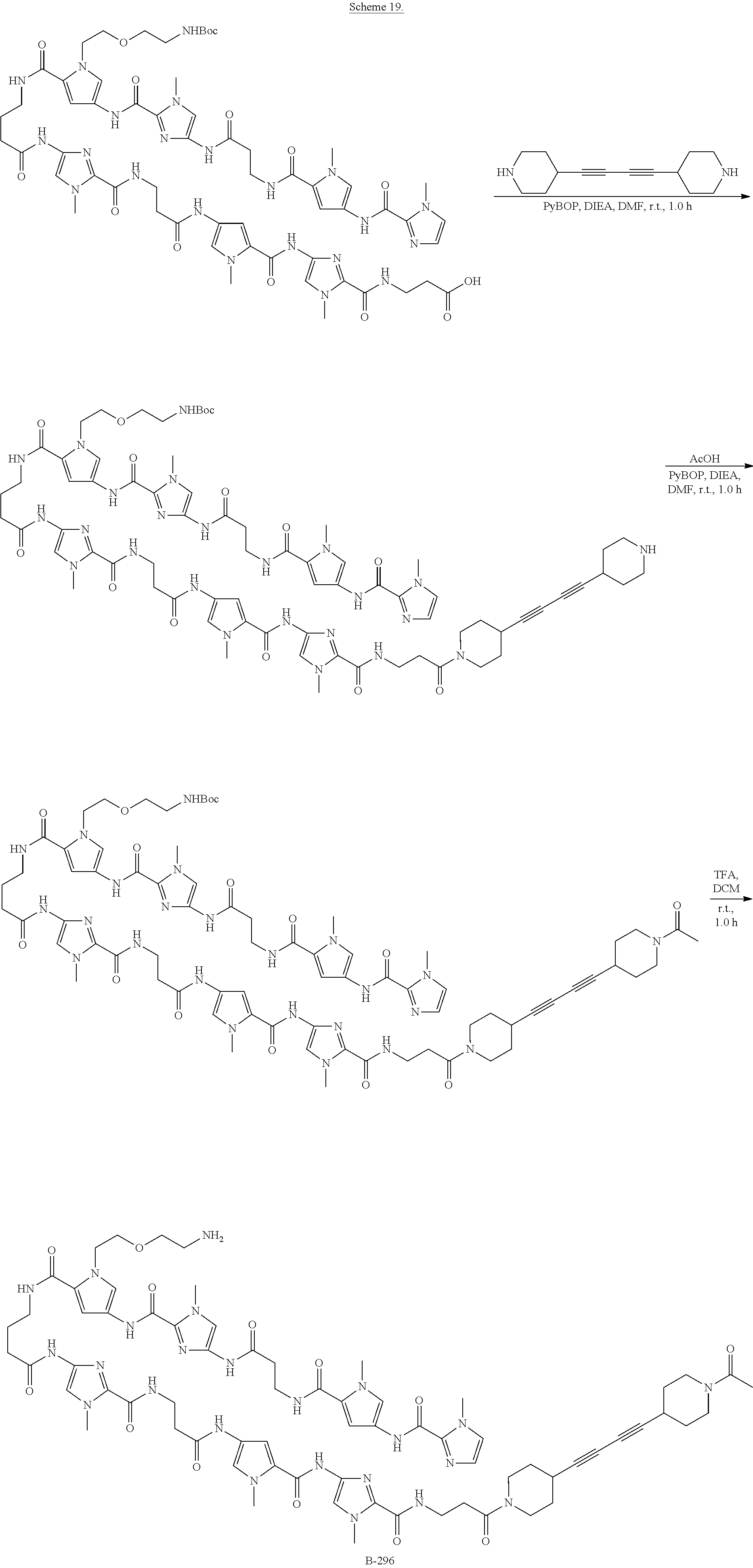

Step 1: The procedure was the same as ethyl 1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylate (Example 2 step 9). 3-({4-[4-(3-{[4-(4-{[1-(2-{2-[(tert-butoxycarbonyl)amino]ethoxy}ethyl)-4-[1-methyl-4-(3-{[1-methyl-4-(1-methylimidazole-2-amido)pyrrol-2-yl]formamido}propanamido)imidazole-2-amido]pyrrol-2-yl]formamido}butanamido)-1-methylimidazol-2-yl]formamido}propanamido)-1-methylpyrrole-2-amido]-1-methylimidazol-2-yl}formamido)propanoic acid (180.00 mg) was used to afford 180.00 mg of the desired product as a yellow solid (60.94%). LC/MS: mass calcd. C₇₃H₉₄N₂₄O₁₄: 1530.74, found: 766.75[M/2+H]⁺.

Step 2: The procedure was the same as Example 9, Compound B-1. Tert-butyl N-{2-[2-(2-{[3-({1-methyl-2-[(2-{[1-methyl-5-({1-methyl-2-[(3-oxo-3-{4-[4-(piperidin-4-yl)buta-1,3-diyn-1-yl]piperidin-1-yl}propyl)carbamoyl]imidazol-4-yl}carbanoyl)pyrrol-3-yl]carbamoyl}ethyl)carbamoyl]imidazol-4-yl}carbamoyl)propyl]carbamoyl}-4-[1-methyl-4-(3-{[1-methyl-4-(1-methylimidazole-2-amido)pyrrol-2-yl]formamido}propanamido)imidazole-2-amido]pyrrol-1-yl)ethoxy]ethyl}carbamate (140.00 mg) was used to afford 70.00 mg of the desired product as a brown solid (46.23% yield). LC/MS: mass calcd. C₇₅H₉₆N₂₄O₁₅: 1572.75, found: 787.65[M/2+H]⁺

Step 3: The procedure was the same as Example 2 step 16, but the reaction mixture was purified by Perp-HPLC. Tert-butyl N-{2-[2-(2-{[3-({2-[(2-{[5-({2-[(3-{4-[4-(1-acetylpiperidin-4-yl)buta-1,3-diyn-1-yl]piperidin-1-yl}-3-oxopropyl)carbamoyl]-1-methylimidazol-4-yl}carbamoyl)-1-methylpyrrol-3-yl]carbamoyl}ethyl)carbamoyl]-1-methylimidazol-4-yl}carbamoyl)propyl]carbamoyl}-4-[1-methyl-4-(3-{[1-methyl-4-(1-methylimidazole-2-amido)pyrrol-2-yl]formamido}propanamido)imidazole-2-amido]pyrrol-1-yl)ethoxy]ethyl}carbamate (56.00 mg) was used to afford 13.2 mg of the desired product a white solid (25.10% yield). HRMS: mass calcd. C₇₀H₈₈N₂₄O₁₃: 1472.6963, found: 1473.7025[M+H]⁺.

### Example 20. Synthesis of Compound B-435

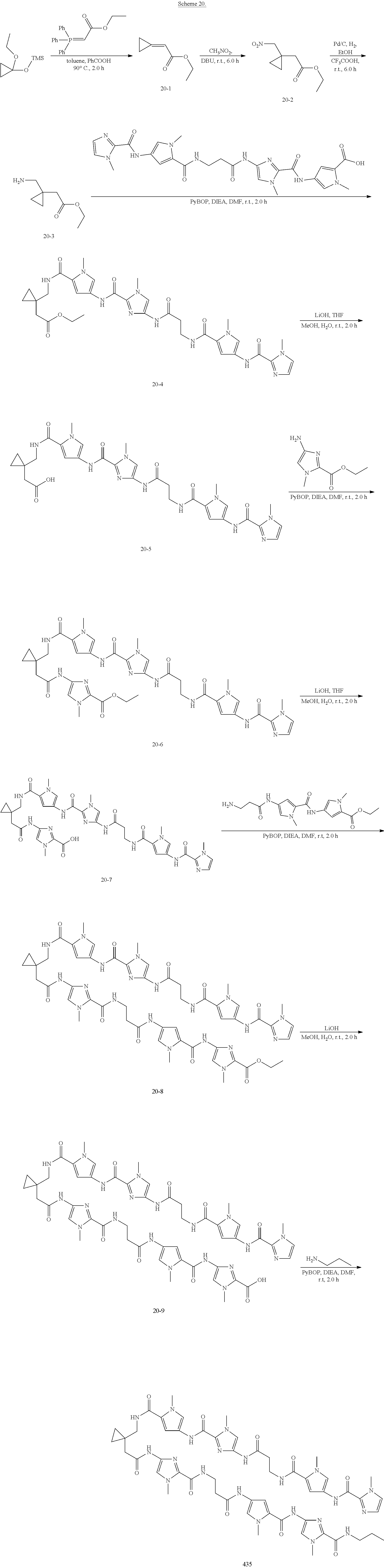

Step 1: To a stirred solution of (1-ethoxycyclopropoxy)trimethylsilane (10.00 g, 57.37 mmol, 1.00 equiv) in toluene (100.00 mL) were added ethyl 2-(triphenyl-lambda5-phosphanylidene)acetate (19.98 g, 57.37 mmol, 1.00 equiv) and benzoic acid (7.00 g, 57.37 mmol, 1.00 equiv) at room temperature. The reaction mixture was stirred for 2.0 h at 90° C. The solid was filtered out. The filtrate was concentrated under vacuum to remove part of the solvent. The resulting mixture was used for the next step directly without further purification. LC/MS: mass calcd. For C₇H₁₀O₂: 126.07, found: 127.10 [M+H]⁺.

Step 2: To a stirred mixture of crude ethyl 2-cyclopropylideneacetate were added CH₃NO₂ (4.01 mL, 74.83 mmol, 2.36 equiv) and DBU (1.99 mL, 13.32 mmol, 0.42 equiv) dropwise at 0° C. The reaction mixture was stirred for 6.0 h at room temperature. The resulting mixture was concentrated under reduced pressure. The residue was purified using silica gel column chromatography and eluted with ethyl acetate/petroleum ether (1:8) to afford ethyl 2-[1-(nitromethyl)cyclopropyl]acetate (3.00 g, 50.54%) as a light yellow oil. LC/MS: mass calcd. For C₈H₁₃NO₄: 187.08, found: 188.20 [M+H]⁺.

Step 3: To a stirred mixture of ethyl 2-[1-(nitromethyl)cyclopropyl]acetate (3.00 g, 16.03 mmol, 1.00 equiv) in EtOH (30.00 mL) was added Pd/C (0.30 g, 10% w/w) and TFA (0.10 mL) at room temperature. The mixture was stirred 6.0 h at room temperature under an atmosphere of hydrogen. The resulting mixture was filtered and the filter cake was washed with EtOH (10 mL×5). The filtrate was concentrated under reduced pressure to give the ethyl 2-[1-(aminomethyl)cyclopropyl]acetate (1.50 g, 59.54%) as a light yellow oil. LC/MS: mass calcd. For C₈H₁₅NO₂: 157.11, found: 158.15 [M+H]⁺.

Step 4: To a stirred mixture of ethyl 2-[1-(aminomethyl)cyclopropyl]acetate (0.50 g, 3.19 mmol, 1.20 equiv), 1-methyl-4-[1-methyl-4-(3-{[1-methyl-4-(1-methylimidazole-2-amido)pyrrol-2-yl]formamido}propanamido) imidazole-2-amido]pyrrole-2-carboxylic acid (1.50 g, 2.66 mmol, 1.00 equiv) and PyBOP (1.66 g, 3.19 mmol, 1.20 equiv) in DMF (20.00 mL) was added DIEA (1.03 g, 7.97 mmol, 3.00 equiv) at room temperature. The reaction mixture was stirred at room temperature for 2.0 h. The reaction was quenched with water (50 mL) at room temperature. The resulting mixture was extracted with EA (30 mL×3). The combined organic layers were washed with water (30 mL×3) and dried over anhydrous Na₂SO₄. After filtration, the filtrate was concentrated under reduced pressure to give ethyl 2-{1-[({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)methyl]cyclopropyl}acetate (1.70 g, 90.92%) as a light yellow solid. LC/MS: mass calcd. For C₃₃H₄₁N₁₁O₇: 703.32, found: 704.25 [M+H]⁺.

Step 5: To a stirred mixture of ethyl 2-{1-[({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)methyl]cyclopropyl}acetate (1.70 g, 2.42 mmol, 1.00 equiv) in MeOH (3 mL) and THF (15.00 mL) was added 2 M LiOH in water (2M, 7.26 mL, 14.52 mmol, 6.00 equiv) at room temperature. The reaction mixture was stirred at room temperature for 2.0 h. The solvent was removed under reduced pressure and the residue was dissolved in H₂O (20 mL). The mixture was acidified to pH 3~5 with 2 M HCl at 0° C. The precipitated solids were collected by filtration, washed with H₂O (3×30 mL), and dried under vacuum. The desired product, {1-1[1(-methyl-4-[1-methyl-4-(3-{[1-methyl-4-(1-methylimidazole-2-amido)pyrrol-2-yl]formamido}propanamido)imidazole-2-amido]pyrrol-2-yl}formamido)methyl]cyclopropyl}acetic acid (1.50 g, 86.79%) was obtained as a light yellow solid. LC/MS: mass calcd. For C₃₁H₃₇N₁₁O₇: 675.29, found: 338.85 [M/2+H]f.

Step 6: To a stirred mixture of {1-[({1-methyl-4-[1-methyl-4-(3-{[1-methyl-4-(1-methylimidazole-2-amido) pyrrol-2-yl]formamido}propanamido)imidazole-2-amido]pyrrol-2-yl}formamido)methyl]cyclopropyl}acetic acid (0.97 g, 1.43 mmol, 1.00 equiv), ethyl 4-amino-1-methylimidazole-2-carboxylate (0.29 g, 1.72 mmol, 1.20 equiv) and PyBOP (0.89 g, 1.72 mmol, 1.20 equiv) in DMF (10.00 mL) was added DIEA (0.56 g, 4.30 mmol, 3.00 equiv) at room temperature. The resulting mixture was stirred for 2.0 h at room temperature. The reaction was poured into water (50 mL). The precipitated solids were collected by filtration and the filter cake was washed with H₂O (50 mL×3), dried under vacuum. The precipitated solids were collected by filtration and washed with water (30 mL×3), dried under vacuum to give ethyl 1-methyl-4-(2-{1-[({1-methyl-4-[1-methyl-4-(3-{[1-methyl-4-(1-methylimidazole-2-amido)pyrrol-2-yl]formamido}propanamido) imidazole-2-amido]pyrrol-2-yl}formamido)methyl]cyclopropyl}acetamido)imidazole-2-carboxylate (600.00 mg, 50.65%) as a light yellow solid. LC/MS: mass calcd. For C₃₈H₄₆N₁₄O: 826.36, found: 827.40 [M+H]⁺.

Step 7: To a stirred mixture of ethyl 1-methyl-4-(2-{1-1({1-methyl-4-|1-methyl-4-(3-{[1-methyl-4-(1-methylimidazole-2-amido)pyrrol-2-yl]formamido}propanamido)imidazole-2-amido]pyrrol-2-yl}formamido) methyl]cyclopropyl}acetamido)imidazole-2-carboxylate (600.00 mg, 0.73 mmol, 1.00 equiv) in MeOH (2.00 mL) and THF (10.00 mL) was added 2 M LiOH in water (2.19 mL, 4.38 mmol, 6.00 equiv) at room temperature. The reaction mixture was stirred at room temperature for 2.0 h. The solvent was removed under reduced pressure. The residue was dissolved in H₂O (10 mL). The mixture was acidified to pH 3~5 with 2 M HCl at 0° C. The precipitated solids were collected by filtration and washed with water (5 mL×5), dried under vacuum to give 1-methyl-4-(2-{1-[({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido) methyl]cyclopropyl}acetamido)imidazole-2-carboxylic acid (500.00 mg, 86.26%) as a light yellow solid. LC/MS: mass calcd. For C₃₆H₄₂N₄O₈: 798.33, found: 799.35 [M+H]⁺.

Step 8: To a stirred mixture of 1-methyl-4-(2-{1-[({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido) methyl]cyclopropyl}acetamido)imidazole-2-carboxylic acid (170.00 mg, 0.21 mmol, 1.00 equiv), ethyl 4-[4-(3-aminopropanamido)-1-methylpyrrole-2-amido]-1-methylimidazole-2-carboxylate (84.83 mg, 0.23 mmol, 1.10 equiv) and PyBOP (132.90 mg, 0.26 mmol, 1.20 equiv) in DMF (2.00 mL) was added DIEA (82.52 mg, 0.64 mmol, 3.00 equiv) at room temperature. The resulting mixture was stirred for 2.0 h at room temperature. The reaction was poured into water (10 mL). The precipitated solids were collected by filtration and the filter cake was washed with H₂O (5 mL×3), dried under vacuum. Ethyl 1-methyl-4-[1-methyl-4-(3-{[1-methyl-4-(2-{1-[(1-methyl-4-[1-methyl-4-(3-{[1-methyl-4-(1-methylimidazole-2-amido)pyrrol-2-yl]formamido}propanamido)imidazole-2-amido}pyrrol-2-yl}formamido)methyl]cyclopropyl}acetamido)imidazol-2-yl]formamido}propanamido)pyrrole-2-amido]imidazole-2-carboxylate (200.00 mg, 82.21%) was obtained as a light yellow solid. LC/MS: mass calcd. For C₅₂H₆₂N₂₀O₁₁: 1142.49, found: 1143.50 [M+H]⁺.

Step 9: To a stirred mixture of ethyl 1-methyl-4-[1-methyl-4-(3-{[1-methyl-4-(2-{1-[({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)methyl]cyclopropyl}acetamido)imidazol-2-yl]formamido}propanamido) pyrrole-2-amido]imidazole-2-carboxylate (180.00 mg, 0.16 mmol, 1.00 equiv) in MeOH (2.00 mL) and THF (10.00 mL) was added 2 M LiOH in water (2.38 mL, 4.77 mmol, 6.00 equiv) at room temperature. The reaction mixture was stirred at room temperature for 2.0 h. The solvent was removed under reduced pressure. The residue was dissolved in H₂O (10 mL). The mixture was acidified to pH 3~5 with 2 M HCl at 0° C. The precipitated solids were collected by filtration and washed with water (5 mL×5), dried under vacuum to give the 1-methyl-4-[1-methyl-4-(3-{[1-methyl-4-(2-{1-[({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)methyl]cyclopropyl}acetamido)imidazol-2-yl]formamido}propanamido)pyrrole-2-amido]imidazole-2-carboxylic acid (150.00 mg, 85.43%) as an off-white solid. LC/MS: mass calcd. For C₅₀H₅₈N₂₀O₁₁: 1114.46, found: 1115.50 [M+H]⁺.

Step 10: To a stirred mixture of 1-methyl-4-|1-methyl-4-(3-{[1-methyl-4-(2-{1-1({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)methyl]cyclopropyl}acetamido)imidazol-2-yl]formamido}propanamido)pyrrole-2-amido]imidazole-2-carboxylic acid (70.00 mg, 0.06 mmol, 1.00 equiv), propylamine (4.45 mg, 0.08 mmol, 1.20 equiv) and PyBOP (39.20 mg, 0.08 mmol, 1.20 equiv) in DMF (2.00 mL) was added DIEA (24.34 mg, 0.19 mmol, 3.00 equiv) at room temperature. The resulting mixture was stirred for 2.0 h at room temperature. The reaction mixture was purified by Prep-HPLC with the following conditions (Column: XBridge Prep Phenyl OBD Column, 19*250 mm, 5 m; Mobile Phase A: Water (10 mmol/L NH₄HCO₃+0.1% NH₃·H₂O), Mobile Phase B: ACN; Flow rate: 25 mL/min; Gradient: 18% B to 43% B in 15 min, 43% B; Wave Length: 254 nm; RT1(min): 12; Number of Runs: 4) to afford 1-methyl-4-(3-{[1-methyl-4-(1-methylimidazole-2-amido)pyrrol-2-yl]formamido}propanamido)-N-[1-methyl-5-({[1-({[1-methyl-2-({2-[(1-methyl-5-{[1-methyl-2-(propylcarbamoyl)imidazol-4-yl]carbamoyl}pyrrol-3-yl)carbamoyl]ethyl}carbamoyl)imidazol-4-yl]carbamoyl}methyl)cyclopropyl]methyl}carbamoyl)pyrrol-3-yl]imidazole-2-carboxamide (5.20 mg, 7.11%) as a white solid. HRMS: mass calcd. For C₅₃H₆₅N₂₁O₁₀: 1155.5223, found: 1156.5264 [M+H]⁺. HPLC: 99.190% purity.

### Example 21. Synthesis of Compound B-439

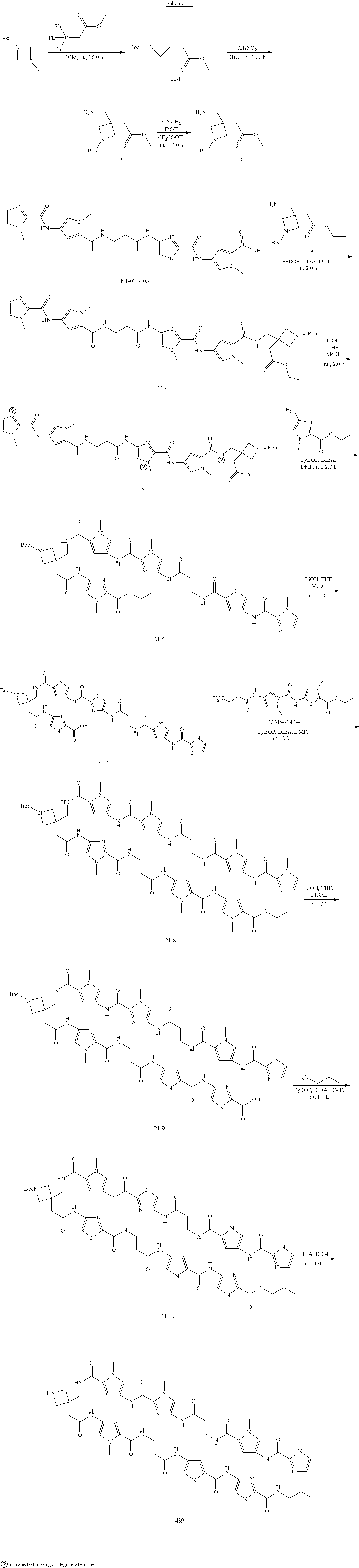

Step 1: To a stirred solution of tert-butyl 3-oxoazetidine-1-carboxylate (5.00 g, 29.21 mmol, 1.00 equiv) in DCM (15.00 mL) was added ethyl 2-(triphenyl-lambda5-phosphanylidene)acetate (10.17 g, 29.21 mmol, 1.00 equiv) in portions at room temperature. The reaction mixture was stirred at room temperature for 16.0 h. The reaction was then quenched by the addition of 40 mL of water. The resulting solution was extracted with extracted with CH₂Cl₂ (50 mL×3). The organic layers were combined, washed with sodium carbonate (aq.) (50 mL×2) and brine (50 mL), dried over anhydrous Na₂SO₄ and concentrated under vacuum. The residue was applied on a silica gel column and eluted with ethyl acetate/petroleum ether (5:1) to afford tert-butyl 3-(2-ethoxy-2-oxoethylidene)azetidine-1-carboxylate (7.00 g, 99.34%) as a colorless oil. LC/MS: mass calcd. For C₁₂H₁₉NO₄: 241.13, found: 242.20 [M+H]⁺. ¹H NMR (300 MHz, Chloroform-d) δ 5.78-5.80 (m, 1H), 4.82-4.85 (m, 2H), 4.59-4.62 (m, 2H), 4.16-4.23 (m, 2H), 1.47 (s, 9H), 1.30 (t, J=7.2 Hz, 3H).

Step 2: To a stirred solution of tert-butyl 3-(2-ethoxy-2-oxoethylidene)azetidine-1-carboxylate (3.70 g, 15.33 mmol, 1.00 equiv) in CH₃NO₂ (5.00 mL) was added DBU (0.50 mL, 3.37 mmol, 0.22 equiv) in portions at room temperature. The reaction mixture was stirred at room temperature for 16.0 h. The resulting mixture was concentrated under vacuum. To the mixture was added EA (30 mL). The resulting mixture was washed with 0.5 N HCl (10 mL×4), dried over Na₂SO₄. The solid was filtered out and filtrate was concentrated. The residue was purified by silica gel column chromatography, eluted with ethyl acetate/petroleum ether (1:10) to afford tert-butyl 3-(2-ethoxy-2-oxoethyl)-3-(nitromethyl)azetidine-1-carboxylate (4.00 g, 82.72%) as a colorless oil. LC/MS: mass calcd. For C₁₃H₂₂N₂O₆: 302.15, found: 247.10 [M−56+H]⁺.

Step 3: To a stirred solution of tert-butyl 3-(2-methoxy-2-oxoethyl)-3-(nitromethyl)azetidine-1-carboxylate (3.90 g, 13.53 mmol, 1.00 equiv) in EtOH (50.00 mL) were added Pd/C (1.00 g, 26% w/w) and TFA (0.10 mL) at room temperature. The flask was evacuated and flushed three times with nitrogen, followed by flushing with hydrogen. The mixture was stirred 16.0 h at room temperature under an atmosphere of hydrogen. The resulting mixture was filtered, the filter cake was washed with MeOH (5×10 mL). The solid was filtered out and the filtration was concentrated. This resulted in tert-butyl 3-(aminomethyl)-3-(2-methoxy-2-oxoethyl)azetidine-1-carboxylate (3.70 g, crude) as a colorless oil. LC/MS: mass calcd. For C₁₃H₂₄N₂O₄: 272.17, found: 273.05 [M+H]⁺.

Step 4: To a stirred mixture of tert-butyl 3-(aminomethyl)-3-(2-ethoxy-2-oxoethyl)azetidine-1-carboxylate (1.89 g, 6.95 mmol, 2.50 equiv) and 1-methyl-4-[1-methyl-4-(3-{[1-methyl-5-(1-methylimidazole-2-amido)pyrrol-2-yl]formamido}propanamido)imidazole-2-amido]pyrrole-2-carboxylic acid (1.57 g, 2.78 mmol, 1.00 equiv) in DMF (20.00 mL) were added PyBOP (1.88 g, 3.62 mmol, 1.30 equiv) and DIEA (0.90 g, 6.95 mmol, 2.50 equiv) at room temperature. The reaction mixture was stirred at room temperature for 2.0 h. The reaction was poured into water (70 mL). The precipitated solids were collected by filtration and the filter cake was washed with H₂O (50 mL×3), dried under vacuum. This resulted in tert-butyl 3-{[(4-{4-[3-({5-[2-(dimethylamino)acetamido]-1-methylpyrrol-2-yl}formamido)propanamido]-1-methylimidazole-2-amido}-1-methylpyrrol-2-yl)formamido]methyl}-3-(2-ethoxy-2-oxoethyl)azetidine-1-carboxylate (2.00 g, 72.29%) as a yellow solid. LC/MS: mass calcd. For C₃₈H₅₀N₂O₉: 818.38, found: 819.50 [M+H]⁺.

Step 5: To a stirred solution of ethyl 3-{[4-(4-{3-[(4-{4-[(4-{4-[(2S)-2-hydroxy-3-{[1-methyl-4-(1-methylimidazole-2-amido)pyrrol-2-yl]formamido}propanamido]-1-methylimidazole-2-amido}-1-methylpyrrol-2-yl)formamido]butanamido}-1-methylimidazol-2-yl)formamido]propanamido}-1-methylpyrrole-2-amido)-1-methylimidazol-2-yl]formamido}propanoate (1.90 g, 2.32 mmol, 1.00 equiv) in MeOH (5.00 mL) and THF (25.00 mL) was added 2 M LiOH in water (6.96 mL, 13.93 mmol, 6.00 equiv) at room temperature. The reaction mixture was stirred at room temperature for 2.0 h. The solvent was removed under reduced pressure. The residue was dissolved in H₂O (20 mL). The mixture was acidified to pH 3~5 with 2 M HCl at 0° C. The precipitated solids were collected by filtration and washed with H₂O (3×30 mL), dried under vacuum. This resulted in 3-{[4-(4-{3-[(4-{4-[(4-{4-[(2S)-2-hydroxy-3-{[1-methyl-4-(1-methylimidazole-2-amido)pyrrol-2-yl]formamido}propanamido]-1-methylimidazole-2-amido}-1-methylpyrrol-2-yl)formamido]butanamido}-1-methylimidazol-2-yl)formamido]propanamido}-1-methylpyrrole-2-amido)-1-methylimidazol-2-yl]formamido}propanoic acid (1.50 g, 81.68%) as a yellow solid. LC/MS: mass calcd. For C₃₆H₄₆N₁₂O₉: 790.35, found: 791.45 [M+H]⁺.

Step 6: To a stirred mixture of [1-(tert-butoxycarbonyl)-3-[({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido) methyl]azetidin-3-yl]acetic acid (1.50 g, 1.90 mmol, 1.00 equiv) and ethyl 4-amino-1-methylimidazole-2-carboxylate (0.80 g, 4.74 mmol, 2.50 equiv) in DMF (20.00 mL) were added HATU (0.94 g, 2.47 mmol, 1.30 equiv) and DIEA (0.74 g, 5.69 mmol. 3.00 equiv). The reaction mixture was stirred at room temperature for 2.0 h. The reaction mixture was filtered and the filtration in DMF (22 mL) was purified by reverse phase column under the conditions: column, C18 silica gel; mobile phase, MeCN in Water (0.1% TFA), 10% to 50% gradient in 10 min; detector, UV 254 mu. This resulted in ethyl 4-{2-[1-(tert-butoxycarbonyl)-3-[({1-methyl-4-[1-methyl-4-(3-{[1-methyl-4-(1-methylimidazole-2-amido)pyrrol-2-yl]formamido}propanamido) imidazole-2-amido]pyrrol-2-yl}formamido)methyl]azetidin-3-yl]acetamido}-1-methylimidazole-2-carboxylate (1.50 g, 82.01%) as a yellow oil. LC/MS: mass calcd. For C₄₃H₅₅N₁₅O₁₀: 941.43, found: 942.60[M+H]⁺.

Step 7: To a stirred solution of ethyl 4-{2-| 1-(tert-butoxycarbonyl)-3-|({1-methyl-4-|1-methyl-4-(3-{[1-methyl-4-(1-methylimidazole-2-amido)pyrrol-2-yl]formamido}propanamido)imidazole-2-amido]pyrrol-2-yl}formamido)methyl]azetidin-3-yl]acetamido}-1-methylimidazole-2-carboxylate (1.45 g. 1.54 mmol, 1.00 equiv) in MeOH (2.00 mL) and THF (10.00 mL) was added 2 M LiOH in water (4.62 mL, 9.23 mmol, 6.00 equiv) at room temperature. The reaction mixture was stirred at room temperature for 2.0 h. The solvent was removed under reduced pressure. The residue was purified by reversed-phase flash chromatography with the following conditions: column, C18 silica gel; mobile phase, MeCN in water (0.1% TFA), 10% to 50% gradient in 10 min; detector, UV 254 nm. This resulted in 4-{2-[1-(tert-butoxycarbonyl)-3-[({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)methyl]azetidin-3-yl]acetamido}-1-methylimidazole-2-carboxylic acid (1.30 g, 86.09%) as a yellow oil. LC/MS: mass calcd. For C₄₁H₅₁N₁₅O₁₀: 913.39, found: 914.50 [M+H]⁺.

Step 8: To a stirred mixture of 4-{2-[1-(tert-butoxycarbonyl)-3-[({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)methyl]azetidin-3-yl]acetamido}-1-methylimidazole-2-carboxylic acid (1.30 g, 1.42 mmol, 1.00 equiv) and ethyl 4-[4-(3-aminopropanamido)-1-methylpyrrole-2-amido]-1-methylimidazole-2-carboxylate (0.62 g, 1.71 mmol, 1.20 equiv) in DMF (15.00 mL) were added PyBOP (0.96 g, 1.85 mmol, 1.30 equiv) and DIEA (0.46 g, 3.56 mmol. 2.50 equiv). The reaction mixture was stirred at room temperature for 2.0 h. The reaction mixture was filtered and the filtration in DMF (20.0 mL) was purified by reverse phase column with the following conditions: column, C18 silica gel; mobile phase, MeCN in water (0.1% TFA), 10% to 50% gradient in 10 min; detector, UV 254 n. The fractions were combined and concentrated under vacuum. This resulted in ethyl 4-(4-{3-[(4-{2-[1-(tert-butoxycarbonyl)-3-[({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)methyl]azetidin-3-yl]acetamido}-1-methylimidazol-2-yl)formamido]propanamido}-1-methylpyrrole-2-amido)-1-methylimidazole-2-carboxylate (2.00 g, 73.90%) as a yellow oil. LC/MS: mass calcd. For C₅₇H₇₁O₁₃: 1257.55, found: 630.10[M/2+H]⁺.

Step 9: To a stirred solution of ethyl 4-(4-{3-[(4-{2-[1-(tert-butoxycarbonyl)-3-[({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)methyl]azetidin-3-yl]acetamido}-1-methylimidazol-2-yl)formamido]propanamido}-1-methylpyrrole-2-amido)-1-methylimidazole-2-carboxylate (1.00 g, 0.80 mmol, 1.00 equiv) in MeOH (5.00 mL) and THF (25.00 mL) was added 2 M LiOH in water (2.38 mL, 4.77 mmol, 6.00 equiv) at room temperature. The reaction mixture was stirred at room temperature for 2.0 h. The solvent was removed under reduced pressure. The residue was purified by reverse phase column with the following conditions: column, C18 silica gel; mobile phase, MeCN in water (0.1% TFA), 10% to 50% gradient in 10 min; detector, UV 254 nm. This resulted in 4-(4-{3-[(4-{2-[1-(tert-butoxycarbonyl)-3-[({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)methyl]azetidin-3-yl]acetamido}-1-methylimidazol-2-yl)formamido]propanamido}-1-methylpyrrole-2-amido)-1-methylimidazole-2-carboxylic acid (900.00 mg, 69.96%) as a yellow oil. LC/MS: mass calcd. For C₅₅H₆₇N₂₁O₁₃: 1229.52, found: 1230.55 [M+H]⁺.

Step 10: To a stirred mixture of 4-(4-{3-[(4-{2-[1-(tert-butoxycarbonyl)-3-[({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)methyl]azetidin-3-yl]acetamido}-1-methylimidazol-2-yl)formamido]propanamido}-1-methylpyrrole-2-amido)-1-methylimidazole-2-carboxylic acid (80.00 mg, 0.07 mmol, 1.00 equiv) and propylamine (4.61 mg, 0.08 mmol, 1.20 equiv) in DMF (2.00 mL) were added PyBOP (43.99 mg, 0.09 mmol, 1.40 equiv) and DIEA (21.01 mg, 0.16 mmol, 2.50 equiv) at room temperature. The reaction mixture was stirred at room temperature for 1.0 h. The reaction mixture was purified by reversed-phase flash chromatography with the following conditions: column, C18 silica gel; mobile phase, MeCN in water (0.1% TFA), 10% to 50% gradient in 10 min; detector, UV 254 nm. The fractions were combined and concentrated under vacuum. This resulted in tert-butyl 3-({[1-methyl-2-({2-[(1-methyl-5-{[1-methyl-2-(propylcarbamoyl) imidazol-4-yl]carbamoyl}pyrrol-3-yl)carbamoyl]ethyl}carbamoyl)imidazol-4-yl]carbamoyl}methyl)-3-[({1-methyl-4-[1-methyl-4-(3-{[1-methyl-4-(1-methylimidazole-2-amido)pyrrol-2-yl]formamido}propanamido) imidazole-2-amido]pyrrol-2-yl}formamido)methyl]azetidine-1-carboxylate (40.00 mg, 46.53%) as a yellow oil. LC/MS: mass calcd. For C₅₈H₇₄NO₁₂: 1270.59, found: 1271.65 [M+H]⁺.

Step 11: To a mixture of tert-butyl 3-(2-((1-methyl-2-((3-((1-methyl-5-((1-methyl-2-(propylcarbamoyl)-1H-imidazol-4-yl)carbamoyl)-1H-pyrrol-3-yl)amino)-3-oxopropyl)carbamoyl)-1H-imidazol-4-yl)amino)-2-oxoethyl)-3-((1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)methyl)azetidine-1-carboxylate (35.00 mg. 0.03 mmol, 1.00 equiv) in CH₂Cl₂ (2.00 mL) was added TFA (0.40 mL). The reaction mixture was stirred at room temperature for 1.0 h. The reaction mixture was concentrated under reduced pressure. The crude product in DMF (1.50 mL) was purified by Prep-HPLC with the following conditions (Column: XBridge Shield RP18 OBD Column, 19*250 mm, 10 μm; Mobile Phase A: Water (10 mmol/L NH₄HCO₃), Mobile Phase B: ACN; Flow rate: 25 mL/min; Gradient: 11% B to 31% B in 18 min, 31% B to 31% B in 22 min, 31% B; Wave Length: 254 nm; RT1(min): 21; Number of Runs: 4) to afford 1-methyl-4-(3-{[1-methyl-4-(1-methylimidazole-2-amido)pyrrol-2-yl]formamido}propanamido)-N-[1-methyl-5-({[3-({[1-methyl-2-({2-[(1-methyl-5-{[1-methyl-2-(propylcarbamoyl)imidazol-4-yl]carbamoyl}pyrrol-3-yl)carbamoyl]ethyl}carbamoyl)imidazol-4-yl]carbamoyl}methyl)azetidin-3-yl]methyl}carbamoyl)pyrrol-3-yl]imidazole-2-carboxamide (2.20 mg, 6.39%) as a white solid. HRMS: mass calcd. For C₅₃H₆₆N₂₂O₁₀: 11705332, found: 1171.5489 [M+H]⁺. HPLC: 93.582% purity.

### Example 22. Synthesis of Additional Compounds of the Disclosure

The compounds of the application were made by the methods similar to Examples 1-22. A summary of the analytical data is represented in Table 3.

TABLE 3
Mass spectrometry data for the compounds of the disclosure.

| | | Observed [M + H]+ |
|---|---|---|
| | Calc. | from TOF-HRMS |
| Cmpd. No. | Exact Mass | [m/z] |
| B-1 | 1200.5438 | 1201.5473 |
| B-2 | 1202.5231 | 1203.5302 |
| B-3 | 1215.5547 | 1216.5581 |
| B-4 | 1244.5336 | 1245.5392 |
| B-5 | 1257.5653 | 1258.5694 |
| B-6 | 1266.5656 | 1267.5200 |
| B-7 | 1308.5762 | 1309.5847 |
| B-8 | 1268.5449 | 1269.5499 |
| B-9 | 1251.5547 | 1252.5569 |
| B-10 | 1322.5918 | 1323.6004 |
| B-11 | 1603.7069 | 1604.7176 |
| B-12 | 1469.6701 | 1470.6808 |
| B-13 | 1515.6545 | 1516.6591 |
| B-14 | 1381.6177 | 1382.6231 |
| B-15 | 1471.6282 | 1472.6384 |
| B-16 | 1337.5915 | 1338.5962 |
| B-17 | 1632.8062 | 1633.8073 |
| B-18 | 1532.7538 | 1533.7606 |
| B-19 | 1544.7538 | 1545.7624 |
| B-20 | 1444.7014 | 1445.7097 |
| B-21 | 1663.8120 | 1664.8153 |
| B-22 | 1529.7752 | 1530.7810 |
| B-23 | 1629.8277 | 1630.8316 |
| B-24 | 1529.7752 | 1530.7791 |
| B-25 | 1606.7654 | 1607.7686 |
| B-26 | 1518.7130 | 1519.7215 |
| B-27 | 1546.7542 | 1547.7577 |
| B-28 | 1446.7017 | 1447.7067 |
| B-29 | 1431.6446 | 1432.6538 |
| B-30 | 1410.6806 | 1411.6873 |
| B-31 | 1310.6282 | 1311.6347 |
| B-32 | 1444.6650 | 1445.6714 |
| B-33 | 1243.5860 | 1244.5924 |
| B-34 | 1257.5765 | 1258.5839 |
| B-35 | 1434.6442 | 1435.6519 |
| B-36 | 1298.6282 | 1299.6351 |
| B-37 | 1285.5966 | 1286.6031 |
| B-38 | 1283.6173 | 1284.6285 |
| B-39 | 1228.5751 | 1229.5819 |
| B-40 | 1298.6533 | 1299.6601 |
| B-41 | 1271.6173 | 1272.6241 |
| B-42 | 1273.5966 | 1274.6038 |
| B-43 | 1129.5067 | 1130.5160 |
| B-44 | 1129.5067 | 1130.5167 |
| B-45 | 1255.5608 | 1256.5672 |
| B-46 | 1262.5442 | 1263.5552 |
| B-47 | 1214.5594 | 1215.5672 |
| B-48 | 1228.5387 | 1229.5469 |
| B-49 | 1227.5547 | 1228.5616 |
| B-50 | 1242.5907 | 1243.5978 |
| B-51 | 1202.5231 | 1203.5293 |
| B-52 | 1216.5387 | 1217.5467 |
| B-53 | 1240.5751 | 1241.5823 |
| B-54 | 1256.5700 | 1257.5814 |
| B-55 | 1255.5860 | 1256.5973 |
| B-56 | 1311.6234 | 1312.6327 |
| B-57 | 1285.6329 | 1286.6398 |
| B-58 | 1311.6234 | 1312.6345 |
| B-59 | 1285.6329 | 1286.6427 |
| B-60 | 1311.6234 | 1312.6344 |
| B-61 | 1285.6329 | 1286.6446 |
| B-62 | 1158.4968 | 1159.5052 |
| B-63 | 1186.5281 | 1187.5357 |
| B-64 | 1072.4852 | 1073.4959 |
| B-66 | 1302.5755 | 1303.5867 |
| B-67 | 935.4263 | 936.4356 |
| B-68 | 1427.6133 | 1428.6254 |
| B-69 | 1321.6078 | 1322.6117 |
| B-71 | 1453.6653 | 1454.6718 |
| B-72 | 1313.6391 | 1314.6461 |
| B-73 | 1262.5442 | 1263.5528 |
| B-74 | 1200.5438 | 1201.5540 |
| B-75 | 1255.5608 | 1256.5704 |
| B-76 | 1464.5700 | 1464.6926 |
| B-77 | 1421.6701 | 1422.6812 |
| B-78 | 1436.6698 | 1437.6784 |
| B-79 | 1538.7379 | 1539.7497 |
| B-80 | 1890.9476 | 1891.9434 |
| B-81 | 1641.8012 | 1642.8123 |
| B-82 | 1683.8118 | 1684.8224 |
| B-83 | 1200.5438 | 1201.5502 |
| B-84 | 1255.5608 | 1256.5688 |
| B-85 | 1647.8382 | 1648.8476 |
| B-86 | 1559.7858 | 1560.7919 |
| B-87 | 1647.8382 | 1648.8456 |
| B-88 | 1605.8277 | 1606.8372 |
| B-89 | 1675.8695 | 1676.8749 |
| B-90 | 1689.8852 | 1690.8914 |
| B-91 | 1709.8539 | 1710.8607 |
| B-92 | 1765.9165 | 1766.9275 |
| B-93 | 1710.8491 | 1711.8541 |
| B-94 | 1710.8491 | 1711.8545 |
| B-95 | 1716.8961 | 1717.9016 |
| B-96 | 1730.9117 | 1731.9151 |
| B-97 | 1515.7596 | 1516.7660 |
| B-98 | 1427.7072 | 1428.7125 |
| B-99 | 1709.8539 | 1710.8607 |
| B-100 | 1710.8491 | 1711.8561 |
| B-101 | 1716.8961 | 1717.9010 |
| B-102 | 1515.7596 | 1516.7686 |
| B-103 | 1427.7072 | 1428.7163 |
| B-104 | 1171.5536 | 1172.5651 |
| B-105 | 1145.5016 | 1146.5068 |
| B-106 | 1239.5911 | 1240.6013 |
| B-107 | 1461.7167 | 1462.7228 |
| B-108 | 1172.5489 | 1173.5592 |
| B-109 | 1363.6071 | 1364.6152 |
| B-110 | 1608.7546 | 1609.7641 |
| B-111 | 1508.7021 | 1509.7128 |
| B-112 | 1197.5077 | 1198.5133 |
| B-113 | 1239.5547 | 1240.5675 |
| B-114 | 1324.6438 | 1325.652 |
| B-115 | 1366.6544 | 1367.6619 |
| B-116 | 1338.6595 | 1339.6673 |
| B-117 | 1310.6282 | 1311.6358 |
| B-118 | 1239.5911 | 1240.5999 |
| B-119 | 1352.6388 | 1353.6438 |
| B-120 | 1324.6438 | 1325.652 |
| B-121 | 1574.7643 | 1575.7718 |
| B-122 | 1602.7956 | 1603.8063 |
| B-123 | 1616.8113 | 1617.822 |
| B-124 | 1574.8007 | 1575.8084 |
| B-125 | 1546.7694 | 1547.7788 |
| B-126 | 1357.6329 | 1358.6396 |
| B-127 | 1324.6438 | 1325.6494 |
| B-128 | 1366.6544 | 1367.6595 |
| B-129 | 1338.6595 | 1339.6672 |
| B-130 | 1352.6388 | 1353.6464 |
| B-131 | 1324.6438 | 1325.6499 |
| B-132 | 1311.5547 | 1312.5606 |
| B-133 | 1339.586 | 1340.5929 |
| B-134 | 1296.5438 | 1297.5552 |
| B-135 | 1315.586 | 1316.5945 |
| B-136 | 1272.5438 | 1273.5505 |
| B-137 | 1303.586 | 1304.5942 |
| B-138 | 1317.6016 | 1318.6114 |
| B-139 | 1339.586 | 1340.5938 |
| B-140 | 1241.5703 | 1242.5798 |
| B-141 | 1283.5809 | 1284.5865 |
| B-142 | 1281.6016 | 1282.6089 |
| B-143 | 1295.6173 | 1296.6282 |
| B-144 | 1783.8907 | 1784.8971 |
| B-145 | 1783.8907 | 1784.8976 |
| B-146 | 1695.8382 | 1696.8473 |
| B-147 | 1607.7858 | 1608.7971 |
| B-148 | 1607.7858 | 1608.7899 |
| B-149 | 1489.7228 | 1490.73 |
| B-150 | 1567.7631 | 1568.7669 |
| B-151 | 1879.9653 | 1880.9703 |
| B-154 | 1666.8315 | 1667.8345 |
| B-155 | 1488.7388 | 1489.7528 |
| B-156 | 1401.6704 | 1402.6823 |
| B-157 | 1651.8583 | 1652.8657 |
| B-158 | 1761.9427 | 1762.9461 |
| B-159 | 1665.8376 | 1666.8468 |
| B-160 | 1775.922 | 1776.9301 |
| B-161 | 1651.8583 | 1652.8618 |
| B-162 | 1761.9427 | 1762.9475 |
| B-163 | 1665.8376 | 1666.8468 |
| B-164 | 1775.922 | 1776.926 |
| B-169 | 1630.8269 | 1631.8346 |
| B-170 | 1616.8477 | 1617.8543 |
| B-171 | 1630.8269 | 1631.84 |
| B-172 | 1616.8477 | 1617.8546 |
| B-173 | 1648.7834 | 1649.789 |
| B-174 | 1634.8041 | 1635.8101 |
| B-175 | 1692.8426 | 1693.853 |
| B-176 | 1678.8633 | 1679.8651 |
| B-177 | 1602.8109 | 1603.8163 |
| B-178 | 1588.8316 | 1589.8309 |
| B-179 | 1228.5751 | 1229.5814 |
| B-180 | 1290.5907 | 1291.5968 |
| B-181 | 1333.6329 | 1334.6403 |
| B-182 | 1540.7953 | 1541.8023 |
| B-183 | 1526.816 | 1527.8237 |
| B-184 | 1540.7953 | 1541.8009 |
| B-185 | 1526.816 | 1527.8249 |
| B-186 | 1408.7014 | 1409.7098 |
| B-187 | 1394.7221 | 1395.7291 |
| B-188 | 1463.6748 | 1464.6835 |
| B-189 | 1449.6955 | 1450.7005 |
| B-190 | 1401.6592 | 1402.6711 |
| B-191 | 1387.6799 | 1388.6888 |
| B-193 | 1381.6541 | 1382.6634 |
| B-194 | 1403.6384 | 1404.6477 |
| B-195 | 1449.6803 | 1450.6894 |
| B-196 | 1349.6279 | 1350.6353 |
| B-197 | 1571.6959 | 1572.7005 |
| B-198 | 1349.6279 | 1350.6349 |
| B-199 | 1561.7691 | 1562.7715 |
| B-200 | 1503.7272 | 1504.7324 |
| B-201 | 1356.5762 | 1357.5889 |
| B-202 | 1356.5762 | 1345.5802 |
| B-203 | 1184.5237 | 1185.5317 |
| B-204 | 1365.6327 | 1366.6395 |
| B-205 | 1186.5394 | 1187.5483 |
| B-205 | 1242.602 | 1243.6121 |
| B-207 | 1213.5754 | 1214.5828 |
| B-208 | 1212.5802 | 1213.5873 |
| B-209 | 1006.4634 | 1007.4683 |
| B-210 | 1438.7258 | 1439.7348 |
| B-212 | 1049.5056 | 1050.5187 |
| B-213 | 1348.5962 | 1349.6017 |
| B-214 | 1339.5595 | 1340.5676 |
| B-215 | 1348.6326 | 1349.6424 |
| B-216 | 1340.5547 | 1341.5594 |
| B-218 | 1278.5544 | 1279.5623 |
| B-219 | 1638.8168 | 1639.8201 |
| B-220 | 1682.8066 | 1683.8106 |
| B-221 | 1594.7542 | 1595.7582 |
| B-222 | 1753.8437 | 1754.8523 |
| B-223 | 1665.7913 | 1666.7951 |
| B-224 | 1505.649 | 1506.6565 |
| B-225 | 1562.7068 | 1563.7102 |
| B-226 | 2084.1207 | 2085.1213 |
| B-227 | 1984.0683 | 1985.0689 |
| B-228 | 2098.1 | 2099.106 |
| B-229 | 1998.0476 | 1999.054 |
| B-230 | 1799.8955 | 1800.8999 |
| B-231 | 1240.5863 | 1241.5917 |
| B-232 | 1268.6176 | 1269.6243 |
| B-233 | 1474.6868 | 1475.6902 |
| B-234 | 1214.5958 | 1215.6014 |
| B-235 | 1520.7637 | 1521.7759 |
| B-236 | 1548.795 | 1549.7993 |
| B-237 | 1754.8641 | 1755.8688 |
| B-238 | 1591.8008 | 1592.8077 |
| B-239 | 1790.9005 | 1791.9046 |
| B-240 | 1748.8899 | 1749.8937 |
| B-241 | 1595.7957 | 1596.8032 |
| B-242 | 1875.9744 | 1876.9819 |
| B-245 | 1201.5067 | 1202.5118 |
| B-247 | 1258.5645 | 1259.5696 |
| B-248 | 1302.5544 | 1303.558 |
| B-249 | 1302.5544 | 1303.5594 |
| B-250 | 1343.6537 | 1344.658 |
| B-251 | 1385.6642 | 1386.6718 |
| B-252 | 1155.5336 | 1156.5394 |
| B-253 | 1351.6435 | 1352.6492 |
| B-254 | 1160.5853 | 1161.5967 |
| B-255 | 1484.7327 | 1485.7398 |
| B-256 | 1442.7221 | 1443.7221 |
| B-257 | 1850.958 | 1851.9598 |
| B-258 | 1808.9475 | 1809.9473 |
| B-259 | 1864.9373 | 1865.9449 |
| B-260 | 1822.9267 | 1823.9318 |
| B-261 | 1693.8689 | 1694.8771 |
| B-262 | 1352.6872 | 1353.6808 |
| B-263 | 1344.6013 | 1345.6055 |
| B-264 | 1255.5608 | 1256.5647 |
| B-265 | 1255.5608 | 1230.5748 |
| B-266 | 1428.6701 | 1429.6769 |
| B-267 | 1387.6435 | 1388.651 |
| B-268 | 1271.5809 | 1272.5858 |
| B-269 | 1470.6806 | 1471.6855 |
| B-270 | 1428.6701 | 1429.6775 |
| B-271 | 1269.5765 | 1270.5807 |
| B-272 | 1243.586 | 1244.593 |
| B-273 | 1442.6857 | 1443.6938 |
| B-278 | 1283.5921 | 1284.5955 |
| B-279 | 1257.6016 | 1258.6054 |
| B-280 | 1456.7014 | 1457.7074 |
| B-281 | 1415.6748 | 1416.68 |
| B-282 | 1299.6122 | 1300.619 |
| B-283 | 1498.7119 | 1499.7151 |
| B-284 | 1456.7014 | 1457.7067 |
| B-285 | 1297.6078 | 1298.6109 |
| B-286 | 1271.6173 | 1272.6254 |
| B-292 | 1327.6435 | 1328.6515 |
| B-293 | 1526.7432 | 1527.7436 |
| B-294 | 1484.7327 | 1485.7404 |
| B-296 | 1472.6963 | 1473.7025 |
| B-297 | 1430.6857 | 1431.6919 |
| B-298 | 1315.6071 | 1316.6106 |
| B-299 | 1514.7068 | 1515.714 |
| B-300 | 1472.6963 | 1473.7072 |
| B-301 | 1287.6122 | 1288.62 |
| B-302 | 1486.7119 | 1487.7203 |
| B-303 | 1444.7014 | 1445.7098 |
| B-304 | 1329.6228 | 1330.6291 |
| B-305 | 1528.7225 | 1529.7284 |
| B-306 | 1486.7119 | 1487.718 |
| B-307 | 1158.4968 | 1159.5049 |
| B-308 | 1087.4597 | 1088.4681 |
| B-309 | 1172.5125 | 1173.5217 |
| B-310 | 1101.4754 | 1102.4857 |
| B-311 | 1186.5281 | 1187.5356 |
| B-312 | 1115.491 | 1116.5019 |
| B-313 | 1186.5281 | 1187.5375 |
| B-314 | 1115.491 | 1116.5 |
| B-315 | 1186.5281 | 1187.5358 |
| B-317 | 1214.5594 | 1215.5669 |
| B-318 | 1214.5594 | 1215.5665 |
| B-319 | 1228.5751 | 1229.581 |
| B-321 | 1242.5907 | 1243.5971 |
| B-322 | 1256.6064 | 1257.6134 |
| B-323 | 1270.622 | 1271.6291 |
| B-324 | 1243.5496 | 1244.5551 |
| B-325 | 1257.5765 | 1258.5853 |
| B-326 | 1313.6391 | 1314.6466 |
| B-327 | 1172.5125 | 1173.5157 |
| B-328 | 1186.5394 | 1187.5475 |
| B-332 | 1255.4785 | 1226.4724 |
| B-333 | 1232.5336 | 1233.5425 |
| B-336 | 1442.7221 | 1443.7268 |
| B-337 | 1200.5802 | 1201.5887 |
| B-338 | 1404.6588 | 1405.6638 |
| B-339 | 1353.5752 | 1354.5813 |
| B-340 | 1362.6483 | 1363.6538 |
| B-341 | 1358.617 | 1359.622 |
| B-342 | 1354.5704 | 1355.5769 |
| B-343 | 1292.57 | 1293.5763 |
| B-344 | 1738.8692 | 1739.8718 |
| B-345 | 1553.6854 | 1554.6939 |
| B-346 | 1331.6173 | 1332.6247 |
| B-347 | 1431.6697 | 1432.6774 |
| B-348 | 1331.6173 | 1332.6242 |
| B-351 | 1848.9424 | 1849.943 |
| B-353 | 1650.8168 | 1651.826 |
| B-355 | 1721.8539 | 1722.8643 |
| B-356 | 1584.7851 | 1585.7919 |
| B-357 | 1303.6799 | 1304.6872 |
| B-358 | 1203.6275 | 1204.634 |
| B-359 | 1407.7061 | 1408.7129 |
| B-360 | 1443.7061 | 1444.7116 |
| B-361 | 1316.57 | 1317.5744 |
| B-362 | 1316.57 | 1317.5748 |
| B-363 | 1426.7232 | 1427.7259 |
| B-364 | 1326.6707 | 1327.6738 |
| B-365 | 1530.7494 | 1531.7514 |
| B-366 | 1021.4743 | 1022.4818 |
| B-367 | 1064.5165 | 1065.5226 |
| B-368 | 1255.5748 | 1256.5796 |
| B-369 | 1493.6813 | 1494.6875 |
| B-370 | 1479.6657 | 1480.6724 |
| B-371 | 1465.65 | 1466.6569 |
| B-372 | 1373.649 | 1374.6523 |
| B-373 | 1212.5312 | 1228.559 |
| B-374 | 1429.6752 | 1430.6796 |
| B-375 | 1737.7832 | 1738.7887 |
| B-376 | 1442.7068 | 1443.7106 |
| B-377 | 1140.4863 | 1141.4911 |
| B-378 | 1457.6854 | 1458.6929 |
| B-379 | 1399.6435 | 1400.646 |
| B-380 | 1358.617 | 1359.6235 |
| B-381 | 1288.5751 | 1289.5784 |
| B-383 | 1359.6122 | 1360.6194 |
| B-384 | 1359.6122 | 1360.6162 |
| B-385 | 1356.6377 | 1357.6458 |
| B-390 | 1318.6068 | 1319.6109 |
| B-391 | 1404.4817 | 1405.4837 |
| B-392 | 1294.4894 | 1295.4928 |
| B-393 | 1310.5918 | 131.5963 |
| B-394 | 1422.4923 | 1423.4975 |
| B-395 | 1173.4965 | 1174.5023 |
| B-396 | 1186.5281 | 1187.5329 |
| B-397 | 1375.6435 | 1376.6536 |
| B-398 | 1355.6748 | 1356.6787 |
| B-399 | 1345.6329 | 1348.62 |
| B-401 | 1469.7177 | 1472.703 |
| B-402 | 1668.8174 | 1671.8065 |
| B-403 | 1242.5907 | 1243.5992 |
| B-404 | 1298.5918 | 1299.6009 |
| B-405 | 1256.5812 | 1257.5969 |
| B-406 | 1241.5703 | 1242.5851 |
| B-407 | 1372.6438 | 1373.6586 |
| B-408 | 1298.6282 | 1299.6332 |
| B-409 | 1144.5176 | 1145.5248 |
| B-410 | 1201.5754 | 1202.5828 |
| B-411 | 1243.6224 | 1244.6279 |
| B-425 | 1311.6486 | 1312.6555 |
| B-426 | 1283.6173 | 1284.6221 |
| B-427 | 1299.6122 | 1300.6207 |
| B-428 | 1311.6486 | 1312.6521 |
| B-429 | 1269.6016 | 1270.609 |
| B-430 | 1297.6329 | 1298.6403 |
| B-431 | 1244.57 | 1329.6336 |
| B-432 | 1348.5962 | 1349.6046 |
| B-433 | 1268.6064 | 1269.6117 |
| B-434 | 1212.5802 | 1213.5837 |
| B-435 | 1155.5223 | 1156.5264 |
| B-436 | 1226.5958 | 1227.6287 |
| B-437 | 1256.6064 | 1257.6102 |
| B-438 | 1227.5911 | 1228.5985 |
| B-439 | 1170.5332 | 1171.5489 |
| B-440 | 1239.5206 | 1239.5280 |
| B-441 | 1240.6115 | 1241.6683 |

### BIOLOGICAL EXAMPLES

### Example B-1. Activity

Fibroblast: a cell type derived from a skin biopsy of a patient. These cells are not altered genetically, so they serve as a primary cell culture model of disease.

iPSC: induced pluripotent stem cell, a cell type that results as a reprogramming of another cell type (typically skin cells or blood cells) into a more embryonic-like state that enables the development of other cell types to model therapeutic effects of drugs in vitro.

SNP: Single Nucleotide Polymorphism, a variation in a single base pair in a DNA sequence

### Molecular Biology Toolkit:

qPCR primer probe sets:
   RNA input normalization was assessed utilizing human glyceraldehyde 3-phosphate dehydrogenase (hGAPDH) TaqMan assay (ThermoFisher cat #4351370) or Human Cyclophillin (IAPP) TaqMan assay (Thermofisher cat #4351372)
   Total HTT detection was assessed utilizing human Htt TaqMan assay (ThermoFisher cat #4331182)
   Allele-specific detection of human HTT expression in HD cells containing the SNP rs362331C/T (Exon 50): for each assay, allele-specific probes to detect the SNP variant contained locked nucleic acid bases to improve allele discrimination, as compared to unmodified DNA probes.
      362331-F (331 forward primer)
      362331-R (331 reverse primer)
      362331-C probe: TCC CTC ATC+C+AC TGT GT
      362331-T probe: CTC+A+T+C+T+A+C TGT GT
   qPCR is performed using Agpath ID one-step reverse transcriptase polymerase chain reaction (RT-PCR) reagent

Protein measurements is performed via western blots probing with antibody MW1 (polyQ specific) to assess reduction of mtHTT alone. D7F7 (a.a surrounding Pro1218) is used to visualize both wtHTT and mtHTT. Lysates were standardized by DC prior to separation on a 3-8% Tris-acetate gel and transferred via wet transfer method onto nitrocellulose membranes. Blots are probed with the previously mentioned antibodies and complementary fluorescent secondary antibodies and imaged on the Li-Cor Odyssey® DLx Imaging system.

Antibody pairing of 2B7 (a.a. 1-17) and MW1 (polyQ specific) is used to track mtHTT levels while pairing of MAB2166 (a.a. 181-810) and MAB5490 (a.a. 115-129) is employed to track total full-length HTT.

Screening of HD molecules methods: GM09197 and/or GM04022 fibroblasts are cultured in T175 flasks incubated at 37° C. and 5% CO₂. Once confluency is reached, the media is removed, the cells are washed with 1×PBS, and are dissociated using TrypLE™ Express Enzyme. Media is added to the enzyme and collected into a 15-mL conical tube and centrifuged at 500×g for 5 minutes to pellet the cells. Media and enzyme are aspirated using a serological pipette. Cells are resuspended in fresh media and counted using a Countess 3 Automated Cell Counter. Cells are plated at a density of 15,000 cells/well in a tissue culture-treated polystyrene 96-well dish and incubated at 37° C. and 5% CO₂ overnight. The next day, media is removed. 200 μL media/well are added back into the plate. The molecules are formulated to 1 mM and are dispensed using a Multidrop™ Pico 8 Digital Dispenser. After a 48-hour incubation with compounds, media is removed from plates, the cells are washed with 1×PBS, and are lysed in 40 μL per well guanidinium thiocyanate buffer. RNA is isolated and purified in 382-well glass fiber column plates using chaotropic salts. Human mtHTT, wtHTT, and GAPDH mRNA are measured via RT-PCR using the ThermoFisher QuantStudio™ 7 Flex System in a 384-well format. Results of HTT levels are normalized to GAPDH mRNA levels. Normalized HTT mRNA levels are expressed relative to vehicle-treated samples to assess fold change after molecule treatment.

iPSC-Neuron Duration of Action of HD molecules methods: Fibroblasts isolated from HD patients are reprogrammed into iPSCs expanded in the presence of cytokines and transduced with the Sendai virus, a cytoplasmic RNA vector. These iPSCs expressed stem cell markers and have normal karyotypes and express the pluripotent markers Nanog, Tra-1-60, and SSeA-44. iPSC-derived neuron differentiation methodology follows standard protocols for mixed cortical neuron differentiation resulting in immunohistochemical staining of iPS-Neuron of Tuj1 and Map2. iPSC-neuronal precursor cells are plated at 300,000 cells/well in a PLO/Laminin-521 coated culture-treated polystyrene 96-well dish and incubated at 37° C. and 5% CO₂. The next day, media is changed to allow neuron precursor cells to continue maturation into neurons. Four days later, media is refreshed, and cells are treated with mitotic inhibitor to remove any remaining dividing cells, resulting in a pure neuronal culture. Three days later, media is removed and 200 μL media/well are added back into the plate. The molecules are formulated to 1 mM and are dispensed using a Multidrop™ Pico 8 Digital Dispenser. After 96-hour incubation with compounds, media is removed and refreshed and the cells are retreated. After 7 days of compound exposure, media is removed from the plates, and the cells were lysed in 60 μL of Ambion Lysis buffer. RNA is isolated using PureLink™ RNA isolation kits. cDNA is synthesized with Agilent Superscript II kit. Human mtHTT, wtHTT, and GAPDH mRNA are measured via RT-PCR using the ThermoFisher QuantStudio™ 7 Flex System in 384-well format. Results of HTT levels are normalized to GAPDH mRNA levels. Normalized HTT mRNA levels are expressed relative to vehicle-treated samples to assess fold change after treatment with the HD compounds.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A transcription modulator molecule having a structure of Formula (I), or a pharmaceutically acceptable salt thereof: 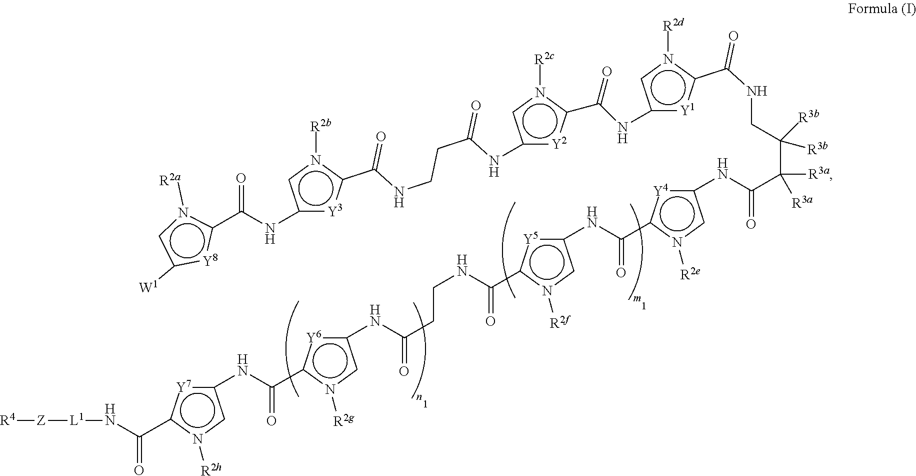
wherein:
W¹ is hydrogen or —N═C(N(R^{1e})₂)₂, wherein each R^{1e} is independently hydrogen or C₁-C₃ alkyl;
each Y¹, Y², Y³, Y⁴, Y⁵, Y⁶, Y⁷, and Y⁸ is independently N or CH;
L¹ is C₁-C₂₀ alkylene or C₁-C₂₀ heteroalkylene;
Z is absent, —C(O)—, or —C(═NH)—;
R⁴ is C₁-C₆ alkyl, —OR^{4b}, or —NR^{4a}R^{4b}; wherein
R^{4a} is hydrogen, optionally substituted C₁-C₂₀ alkyl, or optionally substituted C₁-C₂₀ heteroalkyl;
R^{4b} is optionally substituted C₁-C₂₀ alkyl, optionally substituted C₁-C₂₀ alkenyl, optionally substituted C₁-C₂₀ alkynyl, optionally substituted C₁-C₂₀ aminoalkyl, optionally substituted C₁-C₂₀ haloalkyl, optionally substituted C₁-C₂₀ heteroalkyl, optionally substituted C₁-C₂₀ hydroxyalkyl, optionally substituted C₃-C₅ cycloalkyl, optionally substituted 4 to 8-membered heterocycloalkyl, optionally substituted phenyl, or optionally substituted 5 to 10-membered heteroaryl; or
R^{4a} and R^{4b} together with the nitrogen to which they are attached form an optionally substituted 4 to 8-membered heterocycloalkyl which is partially or fully unsaturated;
each R^{2a}, R^{2b}, R^{2c}, R^{2d}, R^{2e}, R^{2f}, R^{2g}, and R^{2h} is independently hydrogen, optionally substituted C₁-C₅₀ alkyl, optionally substituted C₁-C₅₀ alkenyl, optionally substituted C₁-C₅₀ alkynyl, optionally substituted C₁-C₅₀ heteroalkyl, optionally substituted C₂-C₅₀ heteroalkenyl, optionally substituted C₂-C₅₀ heteroalkynyl, optionally substituted C₁-C₅₀ haloalkyl, optionally substituted C₃-C₈ cycloalkyl, optionally substituted 3 to 8-membered heterocycloalkyl, or optionally substituted PEG₁₋₅₀; each of which is optionally substituted with one or more R¹⁰;
each R^{3a} and R^{3b} is independently hydrogen, halogen, —NR^{11a}R^{11b}, or —NHC(O)R¹², wherein
R^{11a} and R^{11b} are each independently hydrogen, alkyl, or PEG;
R¹² is alkyl, PEG, cycloalkyl, heterocycloalkyl, or phenyl;
or two R^{3a} or two R^{3b} together with the carbon atom to which they are attached form a C₃-C₆ cycloalkyl or 4 to 6-membered heterocycloalkyl;
each R¹⁰ is independently —CN, —OH, —OR^{10a}, —N₃, —NR^{10a}R^{10b}, —CO(O)R^{10a}, —C(O)OR^{10b}, —C(O)NR^{10a}R^{10b}, —NHC(O)R^{10c}, —NHC(O)OR^{10c}, —OC(O)NR^{10a}R^{10b}, or optionally substituted 5 to 10-membered heteroaryl; wherein
R^{10a} and R^{10b} are each independently hydrogen, alkyl, or PEG;
R^{10a} is alkyl, PEG, cycloalkyl, heterocycloalkyl, or phenyl; and
n₁ and m₁ are each independently 0 or 1.

2. The molecule of claim 1, or a pharmaceutically acceptable salt thereof, wherein each Y², Y⁴, Y⁷, and Y⁸ is N.

3. The molecule of claim 1 or 2, or a pharmaceutically acceptable salt thereof, wherein each Y¹, Y³, and Y⁶ is CH.

4. The molecule of claim 1, wherein the molecule has the structure of Formula (Ta), or a pharmaceutically acceptable salt thereof: 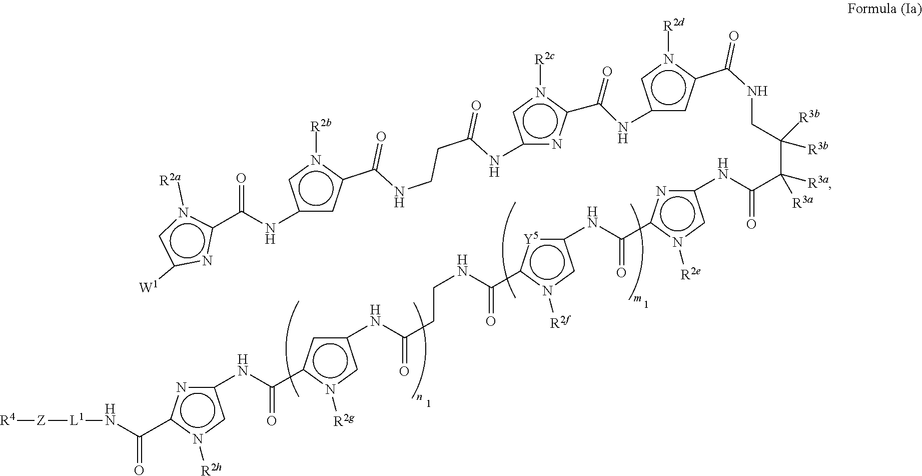
wherein:
W¹ is hydrogen or —N═C(N(R^{1e})₂)₂, wherein each R^{1e} is independently hydrogen or C₁-C₃ alkyl;
each Y⁵ is independently N or CH;
L¹ is C₁-C₂₀ alkylene or C₂-C₂₀ heteroalkylene;
Z is absent, —C(O)—, or —C(═NH)—;
R⁴ is C₁-C₆ alkyl, —OR⁴, or —NR^{4a}R^{4b}, wherein
R^{4a} is hydrogen, optionally substituted C₁-C₂₀ alkyl, or optionally substituted C₁-C₂₀ heteroalkyl;
R^{4b} is optionally substituted C₁-C₂₀ alkyl, optionally substituted C₂-C₂₀ alkenyl, optionally substituted C₂-C₂₀ alkynyl, optionally substituted C₁-C₂₀ aminoalkyl, optionally substituted C₁-C₂₀ haloalkyl, optionally substituted C₁-C₂₀ heteroalkyl, optionally substituted C₁-C₂₀ hydroxyalkyl, optionally substituted C₃-C₈ cycloalkyl, optionally substituted 4 to 8-membered heterocycloalkyl, optionally substituted phenyl, or optionally substituted 5 to 10-membered heteroaryl; or
R^{4a} and R^{4b} together with the nitrogen to which they are attached form an optionally substituted 4 to 8-membered heterocycloalkyl which is partially or fully unsaturated;
each R^{2a}, R^{2b}, R^{2c}, R^{2d}, R^{2e}, R^{2f}, R^{2g}, and R^{2h} is independently hydrogen, optionally substituted C₁-C₅₀ alkyl, optionally substituted C₂-C₅₀ alkenyl, optionally substituted C₂-C₅₀ alkynyl, optionally substituted C₁-C₅₀ heteroalkyl, optionally substituted C₂-C₅₀ heteroalkenyl, optionally substituted C₂-C₅₀ heteroalkynyl, optionally substituted C₁-C₅₀ haloalkyl, optionally substituted C₃-C₈ cycloalkyl, optionally substituted 3 to 8-membered heterocycloalkyl, or optionally substituted PEG₁₋₅₀; each of which is optionally substituted with one or more R¹⁰;
each R^{3a} and R^{3b} is independently hydrogen, halogen, —NR^{11a}R^{11b}, or —NHC(O)R¹², wherein
R^{4a} and R^{4b} are each independently hydrogen, alkyl, or PEG;
R¹² is alkyl, PEG, cycloalkyl, heterocycloalkyl, or phenyl;
or two R^{3a} or two R^{3b} together with the carbon atom to which they are attached form a C₃-C₆ cycloalkyl or 4 to 6-membered heterocycloalkyl;
each R¹⁰ is independently —CN, —OH, —OR^{10a}, —N₃, —NR^{10a}R^{10b}, —CO(O)R^{10c}, —C(O)OR^{10d}, —C(O)NR^{10a}R^{10b}, —NHC(O)R^{10c}, —NHC(O)OR^{10c}c, —OC(O)NR^{10a}R^{10b}, or optionally substituted 5 to 10-membered heteroaryl, wherein
R^{10a} and R^{10b} are each independently hydrogen, alkyl, or PEG;
R^{10c} is alkyl, PEG, cycloalkyl, heterocycloalkyl, or phenyl; and
n₁ and m₁ are each independently 0 or 1.

5. The molecule of any one of claims 1-4, or a pharmaceutically acceptable salt thereof, wherein
L¹ is C₁-C₂₀ alkylene or C₂-C₂₀ heteroalkylene;
Z is absent or —C(O)—; and
R⁴ is —NR^{4a}R^{4b}.

6. The molecule of claim 5, or a pharmaceutically acceptable salt thereof, wherein Z is absent.

7. The molecule of claim 5, or a pharmaceutically acceptable salt thereof, wherein Z is —C(O)—.

8. The molecule of any one of claims 5-7, or a pharmaceutically acceptable salt thereof, wherein L¹ is C₁-C₁₀ alkylene.

9. The molecule of any one of claims 1-8, or a pharmaceutically acceptable salt thereof, wherein:
R^{4a} is hydrogen, optionally substituted C₁-C₂₀ alkyl, or optionally substituted C₁-C₂₀ heteroalkyl; and
R^{4b} is optionally substituted C₁-C₂₀ alkyl or optionally substituted C₁-C₂₀ heteroalkyl.

10. The molecule of claim 9, or a pharmaceutically acceptable salt thereof, wherein R^{4a} is an optionally substituted C₁-C₂₀ heteroalkyl; and R^{4b} is an optionally substituted C₁-C₂₀ heteroalkyl.

11. The molecule of claim 10, or a pharmaceutically acceptable salt thereof, wherein the heteroalkyl is PEG₁₋₁₀.

12. The molecule of claim 9, or a pharmaceutically acceptable salt thereof, wherein R^{4a} is an optionally substituted C₁-C₆ alkyl; and R^{4b} is an optionally substituted C₁-C₆ alkyl.

13. The molecule of claim 1, wherein the molecule has the structure of Formula (II), or a pharmaceutically acceptable thereof: 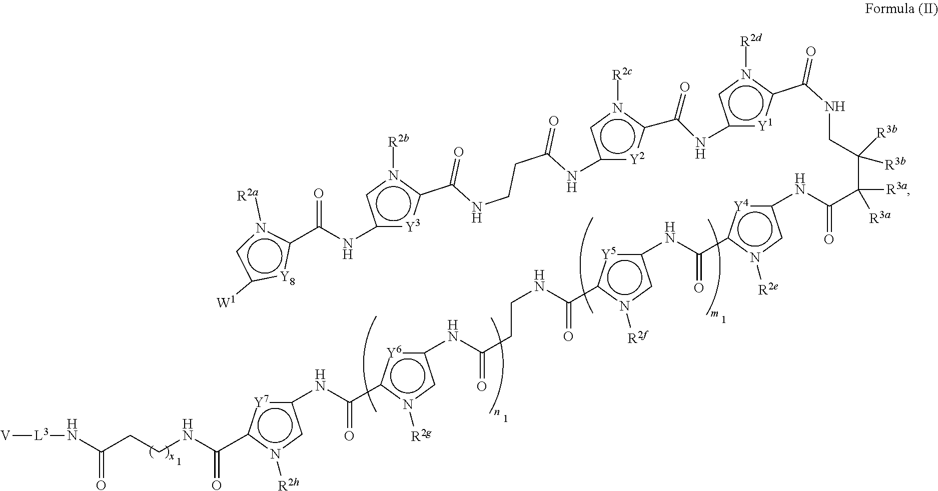
wherein:
W¹ is hydrogen or —N═C(N(R^{1e})₂)₂, wherein each R^{1e} is independently hydrogen or C₁-C₃ alkyl;
each Y¹, Y², Y³, Y⁴, Y⁵, Y⁶, Y⁷, and Y⁸ is independently N or CH;
L³ is C₁-C₂₀ alkylene, C₂-C₂₀ heteroalkylene, or (AA)₁₋₁₀; wherein
each AA is independently a naturally occurring amino acid;
V is absent, optionally substituted C₃-C₈ cycloalkyl, optionally substituted 4 to 8-membered heterocycloalkyl, optionally substituted phenyl, or optionally substituted 5 to 10-membered heteroaryl;
each R^{2a}, R^{2b}, R^{2c}, R^{2d}, R^{2e}, R^{2f}, R^{2g}, and R^{2h} is independently hydrogen, optionally substituted C₁-C₅₀ alkyl, optionally substituted C₂-C₅₀ alkenyl, optionally substituted C₂-C₅₀ alkynyl, optionally substituted C₁-C₅₀ heteroalkyl, optionally substituted C₂-C₅₀ heteroalkenyl, optionally substituted C₂-C₅₀ heteroalkynyl, optionally substituted C₁-C₅₀ haloalkyl, optionally substituted C₃-C₈ cycloalkyl, optionally substituted 3 to 8-membered heterocycloalkyl, or optionally substituted PEG₁₋₅₀; each of which is optionally substituted with one or more R¹⁰;
each R^{3a} and R^{3b} is independently hydrogen, halogen, —NR^{11a}R^{11b}, or —NHC(O)R¹², wherein
R^{11a} and R^{11b} are each independently hydrogen, alkyl, or PEG;
R¹² is alkyl, PEG, cycloalkyl, heterocycloalkyl, or phenyl;
or two R^{3a} or two R^{3b} together with the carbon atom to which they are attached form a C₃-C₆ cycloalkyl or 4 to 6-membered heterocycloalkyl;
each R¹⁰ is independently —CN, —OH, —OR^{10a}, —N₃, —NR^{10a}R^{10b}, —CO(O)R^{10c}, —C(O)OR^{10c}, —C(O)NR^{10a}R^{10b}, —NHC(O)R^{10c}, —NHC(O)OR^{10c}, —OC(O)NR^{10a}R^{10b}, or optionally substituted 5 to 10-membered heteroaryl; wherein
R^{10a} and R^{10b} are each independently hydrogen, alkyl, or PEG;
R^{10c} is alkyl, PEG, cycloalkyl, heterocycloalkyl, or phenyl;
n₁ and m₁ are each independently 0 or 1; and
X¹ is 0-10.

14. The molecule of claim 13, or a pharmaceutically acceptable salt thereof, wherein each Y², Y⁴, Y⁷, and Y⁸ is N.

15. The molecule of claim 13 or 14, or a pharmaceutically acceptable salt thereof, wherein each Y¹, Y³, and Y⁶ is CH.

16. The molecule of any one of claims 1-15, or a pharmaceutically acceptable salt thereof, wherein each R^{3b} is hydrogen; and each R^{3a} is independently selected from hydrogen, —NR^{11a}R^{11b}, and —NHC(O)R¹².

17. The molecule of any one of claims 1-15, or a pharmaceutically acceptable salt thereof, wherein each R^{3a} is hydrogen; and each R^{3b} is independently selected from hydrogen, —NR^{11a}R^{11b}, and —NHC(O)R¹².

18. The molecule of any one of claims 1-15, or a pharmaceutically acceptable salt thereof, wherein each R^{3a} and each R^{3b} are hydrogen.

19. The molecule of any one of claims 1-15, or a pharmaceutically acceptable salt thereof, wherein two R^{3a} together with the carbon atom to which they are attached form a C₃-C₆ cycloalkyl or 4 to 6-membered heterocycloalkyl; and each R^{3b} is hydrogen.

20. The molecule of any one of claims 1-15, or a pharmaceutically acceptable salt thereof, wherein two R^{3b} together with the carbon atom to which they are attached form a C₃-C₆ cycloalkyl or 4 to 6-membered heterocycloalkyl; and each R^{3a} is hydrogen.

21. The molecule of any one of claims 1-20, or a pharmaceutically acceptable salt thereof, wherein each R^{2a}, R^{2b}, R^{2c}, R^{2d}, R^{2e}, R^{2f}, R^{2g}, and R^{2h} is independently hydrogen, optionally substituted C₁-C₅₀ alkyl, optionally substituted C₁-C₅₀ heteroalkyl, optionally substituted C₁-C₅₀ haloalkyl, or optionally substituted PEG₁₋₅₀; each of which is optionally substituted with one or more R¹⁰.

22. The molecule of any one of claims 1-20, or a pharmaceutically acceptable salt thereof, wherein each R^{2a}, R^{2b}, R^{2c}, R^{2d}, R^{2e}, R^{2f}, R^{2g}, and R^{2h} is independently hydrogen, optionally substituted C₁-C₁₀ alkyl, optionally substituted C₁-C₁₀ heteroalkyl, optionally substituted C₁-C₁₀ haloalkyl, or optionally substituted PEG₁₋₁₀; each of which is optionally substituted with one or more R¹⁰.

23. The molecule of any one of claims 1-20, or a pharmaceutically acceptable salt thereof, wherein each R^{2a}, R^{2b}, R^{2c}, R^{2d}, R^{2e}, R^{2f}, R^{2g}, and R^{2h} is independently C₁-C₁₀ alkyl, each of which is optionally substituted with one or more R¹.

24. The molecule of any one of claims 1-20, or a pharmaceutically acceptable salt thereof, wherein each R^{2a}, R^{2b}, R^{2a}, and R^{2g} is independently an optionally substituted C₁-C₁₀ alkyl, optionally substituted C₁-C₁₀ heteroalkyl, optionally substituted C₁-C₁₀ haloalkyl, or optionally substituted PEG₁₋₁₀; each of which is optionally substituted with one or more R¹⁰; and each of R^{2c}, R^{2e}, and R^{2h} is independently unsubstituted C₁-C₁₀ alkyl.

25. The molecule of claim 24, or a pharmaceutically acceptable salt thereof, wherein each of R^{2c}, R^{2e}, and R^{2h} is methyl.

26. The molecule of claim 24 or 25, or a pharmaceutically acceptable salt thereof, wherein each R^{2a}, R^{2b}, and R^{2g} is independently unsubstituted C₁-C₁₀ alkyl.

27. The molecule of claim 26, or a pharmaceutically acceptable salt thereof, wherein each R^{2a}, R^{2b}, and R^{2g} is methyl.

28. The molecule of any one of claims 1-27, or a pharmaceutically acceptable salt thereof, wherein n₁ is 1.

29. The molecule of claim 13, wherein the molecule has the structure of Formula (IIa), or a pharmaceutically acceptable salt thereof: 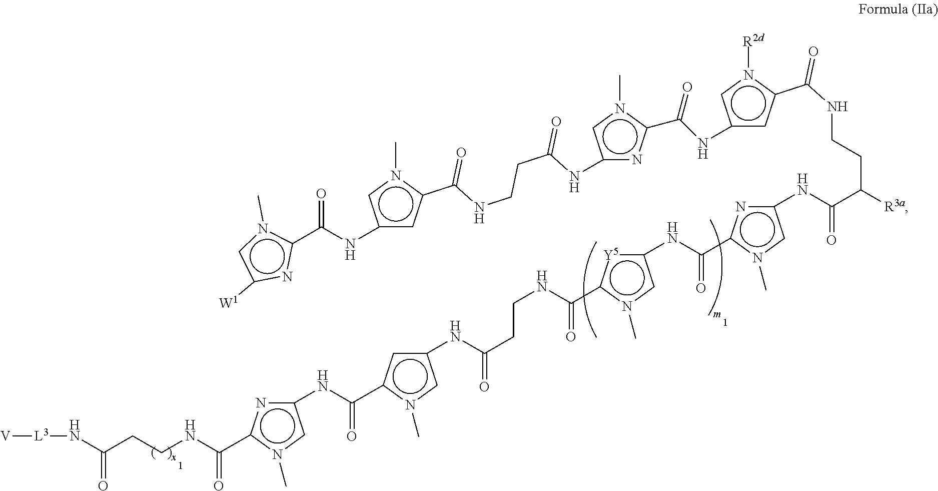
wherein:
W¹ is hydrogen or —N═C(N(R^{1e})₂)₂, wherein each R^{1e} is independently hydrogen or C₁-C₃ alkyl;
each Y⁵ is independently N or CH;
L³ is C₁-C₂₀ alkylene, C₂-C₂₀ heteroalkylene, or AA₁₋₁₀; wherein
each AA is independently a naturally occurring amino acid;
V is absent, optionally substituted C₃-C₈ cycloalkyl, optionally substituted 4 to 8-membered heterocycloalkyl, optionally substituted phenyl, or optionally substituted 5 to 10-membered heteroaryl;
R^{2d} is hydrogen, optionally substituted C₁-C₅₀ alkyl, optionally substituted C₂-C₅₀ alkenyl, optionally substituted C₂-C₅₀ alkynyl, optionally substituted C₁-C₅₀ heteroalkyl, optionally substituted C₂-C₅₀ heteroalkenyl, optionally substituted C₂-C₅₀ heteroalkynyl, optionally substituted C₁-C₅₀ haloalkyl, optionally substituted C₃-C₅ cycloalkyl, optionally substituted 3 to 8-membered heterocycloalkyl, or optionally substituted PEG₁₋₅₀; each of which is optionally substituted with one or more R¹⁰;
R^{3a} is hydrogen, halogen, —NR^{11a}R^{11b}, or —NHC(O)R¹², wherein
R^{11a} and R^{11b} are each independently hydrogen, alkyl, or PEG;
R¹² is alkyl, PEG, cycloalkyl, heterocycloalkyl, or phenyl;
each R¹⁰ is independently —CN, —OH, —OR^{10a}, —N₃, —NR^{10a}R^{10b}, —CO(O)R^{10c}, —C(O)OR^{10c}, —C(O)NR^{10a}R^{10b}, —NHC(O)R^{10c}, —NHC(O)OR^{10c}, —OC(O)NR^{10a}R^{10b}, or optionally substituted 5 to 10-membered heteroaryl; wherein
R^{10a} and R^{10b} are each independently hydrogen, alkyl, or PEG;
R^{10c} is alkyl, PEG, cycloalkyl, heterocycloalkyl, or phenyl;
m₁ is 0 or 1; and
x₁ is 0-10.

30. The molecule of any one of claims 13-29, or a pharmaceutically acceptable salt thereof, wherein V is an optionally substituted C₃-C₈ cycloalkyl, optionally substituted 4 to 8-membered heterocycloalkyl, optionally substituted phenyl, or optionally substituted 5 to 10-membered heteroaryl.

31. The molecule of claim 30, or a pharmaceutically acceptable salt thereof, wherein V is an optionally substituted C₃-C₈ cycloalkyl or optionally substituted 4 to 8-membered heterocycloalkyl.

32. The molecule of claim 30 or 31, or a pharmaceutically acceptable salt thereof, wherein V is an optionally substituted 4 to 8-membered heterocycloalkyl.

33. The molecule of claim 32, or a pharmaceutically acceptable salt, wherein V is piperazine, piperidine, or morpholine.

34. The molecule of any one of claims 13-29, or a pharmaceutically acceptable salt thereof, wherein V has the structure of Formula (C), or a pharmaceutically acceptable salt thereof: 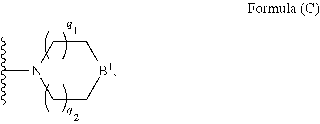
wherein:
B¹ is —CR^{5a}R^{5b}—, —O—, —NR^{5b}—, —S—, —S(O)—, or —S(O)₂—; or B¹ is 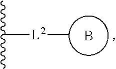
wherein
R^{5a} is hydrogen, —OH, or optionally substituted C₁-C₂₀ alkyl;
R^{5b} is hydrogen, optionally substituted C₁-C₂₀ alkyl, optionally substituted C₂-C₂₀ alkenyl, optionally substituted C₂-C₂₀ alkynyl, optionally substituted C₁-C₂₀ heteroalkyl, —C(O)OR⁶, or —C(O)R⁶; or
R^{5a} and R^{5b} together with the nitrogen atom to which they are attached form an optionally substituted 4 to 8-membered heterocycloalkyl;
R⁶ is hydrogen, optionally substituted C₁-C₂₀ alkyl, optionally substituted C₁-C₁₀ haloalkyl, optionally substituted C₃-C₆ cycloalkyl, optionally substituted 3 to 6-membered heterocycloalkyl, or optionally substituted phenyl;
ring B is absent, optionally substituted C₃-C₆ cycloalkyl, optionally substituted 4 to 6-membered heterocycloalkyl, optionally substituted phenyl, or optionally substituted 5 to 10-membered heteroaryl;
L² is absent, C₁-C₄ alkylene, C₂-C₄ alkynelene, or C₂-C₄ alkynylene; and
q₁ and q₂ are each independently 0, 1, or 2.

35. The molecule of any one of claims 13-29, or a pharmaceutically acceptable salt thereof, wherein V has the structure of Formula (C-1), or a pharmaceutically acceptable salt thereof: 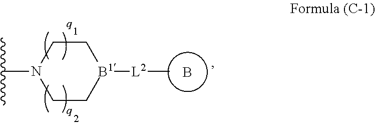
wherein:
ring B is optionally substituted C₃-C₆ cycloalkyl, optionally substituted 4 to 6-membered heterocycloalkyl, optionally substituted phenyl, or optionally substituted 5 to 10-membered heteroaryl;
L² is absent, C₁-C₄ alkylene, C₂-C₄ alkynelene, or C₂-C₄ alkynylene bond, alkylene, or alkynylene;
B¹ is CH or N; and
q₁ and q₂ are each independently 0, 1, or 2.

36. The molecule of any one of claims 13-29, wherein V has the structure of Formula (C-2), or a pharmaceutically acceptable salt thereof: 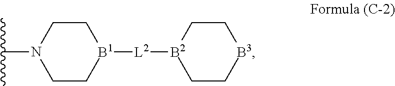
wherein:
B¹ and B² are each independently CH or N; and
B³ is —CR^{7a}R^{7b}—, —O—, —S—, —S(O)—, —S(O)₂—, or —NR^{7b}—; wherein
R^{7a} is hydrogen or optionally substituted C₁-C₂₀ alkyl;
R^{7b} is hydrogen, optionally substituted C₁-C₂₀ alkyl, optionally substituted C₂-C₂₀ alkenyl, optionally substituted C₂-C₂₀ alkynyl, optionally substituted C₁-C₂₀ heteroalkyl, —C(O)OR⁸, or —C(O)R⁸;
R⁸ is hydrogen, optionally substituted C₁-C₂₀ alkyl, optionally substituted C₁-C₁₀ haloalkyl, optionally substituted PEG₁₋₂₀, optionally substituted C₃-C₆ cycloalkyl, optionally substituted 3 to 6-membered heterocycloalkyl, or optionally substituted phenyl; and
L² is absent, C₁-C₄ alkylene, C₂-C₄ alkynelene, or C₂-C₄ alkynylene.

37. The molecule of any one of claims 13-29, or a pharmaceutically acceptable salt thereof, wherein V has the structure of Formula (C-3), or a pharmaceutically acceptable salt thereof: 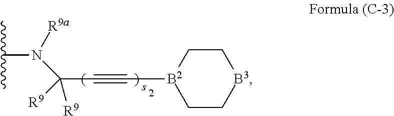
wherein:
B² is CH or N;
B³ is —CR^{7a}R^{7b}—, —O—, —S—, —S(O)—, —S(O)₂—, or —NR^{7b}—; wherein
R^{7a} is hydrogen or optionally substituted C₁-C₂₀ alkyl;
R^{7b} is hydrogen, optionally substituted C₁-C₂₀ alkyl, optionally substituted C₂-C₂₀ alkenyl, optionally substituted C₂-C₂₀ alkynyl, optionally substituted C₁-C₂₀ heteroalkyl, —C(O)OR⁸, or —C(O)R⁸; and
R⁸ is hydrogen, optionally substituted C₁-C₂₀ alkyl, optionally substituted C₁-C₁₀ haloalkyl, optionally substituted PEG₁₋₂₀, optionally substituted C₃-C₆ cycloalkyl, optionally substituted 3 to 6-membered heterocycloalkyl, or optionally substituted phenyl;
R^{9a} is hydrogen, optionally substituted C₁-C₂₀ alkylene, or optionally substituted PEG₁₋₂₀;
each R⁹ is independently hydrogen or C₁-C₃ alkyl; and
s₂ is 1-3.

38. The molecule of claim 1, or a pharmaceutically acceptable salt thereof, wherein R⁴ is —NR^{4a}R^{4b} wherein R^{4a} and R^{4b} together with the nitrogen to which they are attached form an optionally substituted 4 to 8-membered heterocycloalkyl.

39. The molecule of claim 38, or a pharmaceutically acceptable salt thereof, wherein R^{4a} and R^{4b} together with the nitrogen to which they are attached form an optionally substituted piperidine, optionally substituted piperidine, or optionally substituted morpholine.

40. The molecule of claim 13, wherein the molecule has the structure of Formula (III), or a pharmaceutically acceptable salt thereof: 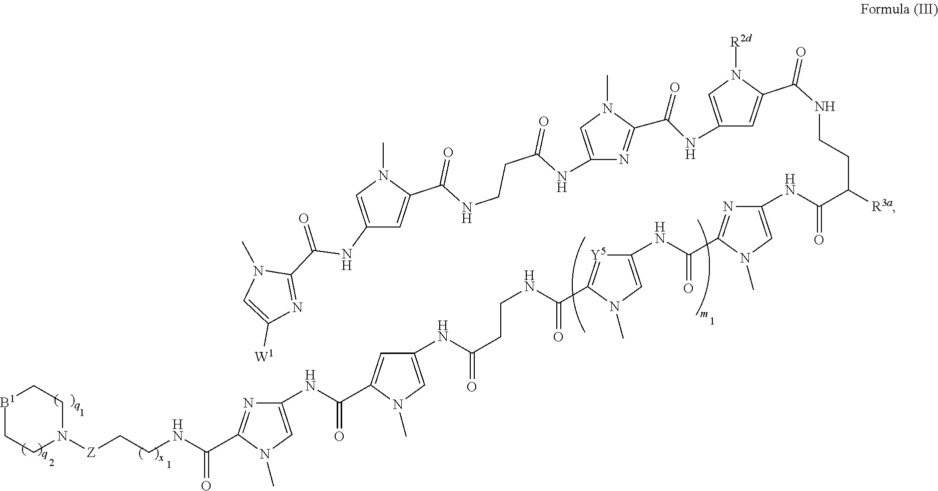
wherein:
B¹ is —CR^{5a}R^{5b}, —O—, —NR^{5b}—, —S—, —S(O)—, or —S(O)₂—; or B¹ is 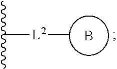
wherein
R^{5a} is hydrogen, —OH, or optionally substituted C₁-C₂₀ alkyl;
R^{5b} is hydrogen, optionally substituted C₁-C₂₀ alkyl, optionally substituted C₂-C₂₀ alkenyl, optionally substituted C₂-C₂₀ alkynyl, optionally substituted C₁-C₂₀ heteroalkyl, —C(O)OR⁶, or C(O)R⁶; or
R^{5a} and R^{5b} together with the nitrogen atom to which they are attached form an optionally substituted 4 to 8-membered heterocycloalkyl;
R⁶ is hydrogen, optionally substituted C₁-C₂₀ alkyl, optionally substituted C₁-C₂₀ haloalkyl, optionally substituted C₃-C₆ cycloalkyl, optionally substituted 3 to 6-membered heterocycloalkyl, or optionally substituted phenyl;
ring B is absent, optionally substituted C₃-C₆ cycloalkyl, optionally substituted 4 to 6-membered heterocycloalkyl, optionally substituted phenyl, or optionally substituted 5 to 10-membered heteroaryl;
L² is absent, C₁-C₄ alkylene, C₂-C₄ alkynelene, or C₂-C₄ alkynylene;
W¹ is hydrogen or —N═C(N(R^{e})₂)₂, wherein each R^{1e} is independently hydrogen or C₁-C₃ alkyl;
Z is absent or C(O);
each Y⁵ is independently N or CH;
R^{2d} is hydrogen, optionally substituted C₁-C₅₀ alkyl, optionally substituted C₂-C₅₀ alkenyl, optionally substituted C₂-C₅₀ alkynyl, optionally substituted C₁-C₅₀ heteroalkyl, optionally substituted C₂-C₅₀ heteroalkenyl, optionally substituted C₂-C₅₀ heteroalkynyl, optionally substituted C₁-C₅₀ haloalkyl, optionally substituted C₃-C₈ cycloalkyl, optionally substituted 3 to 8-membered heterocycloalkyl, or optionally substituted PEG₁₋₅₀; each of which is optionally substituted with one or more R¹⁰;
R^{3a} is hydrogen, halogen, —NR^{11a}R^{11b}, or —NHC(O)R¹², wherein
R^{11a} and R^{11b} are each independently hydrogen, alkyl, or PEG;
R¹² is alkyl, PEG, cycloalkyl, heterocycloalkyl, or phenyl;
each R¹⁰ is independently —CN, —OH, —OR^{10a}, —N₃, —NR^{10a}R^{10b}, —CO(O)R^{10c}, —C(O)OR^{10c}, —C(O)NR^{10a}R^{10b}, —NHC(O)R^{10c}, —NHC(O)OR^{10c}, —OC(O)NR^{10a}R^{10b}, or optionally substituted 5 to 10-membered heteroaryl; wherein
R^{10a} and R^{10b} are each independently hydrogen, alkyl, or PEG;
R^{10c} is alkyl, PEG, cycloalkyl, heterocycloalkyl, or phenyl;
m₁ is 0 or 1;
q₁ and q₂ are each independently 0-2; and
x₁ is 0-10.

41. The molecule of claim 40, or a pharmaceutically acceptable salt thereof, wherein q₁ is 1 and q₂ is 1.

42. The molecule of claim 40 or 41, or a pharmaceutically acceptable salt thereof, wherein B¹ is —O— or —NR^{5b}—.

43. The molecule of claim 40 or 41, or a pharmaceutically acceptable salt thereof, wherein B¹ is —CR^{5a}R^{5b}—.

44. The molecule of claim 40 or 41, or a pharmaceutically acceptable salt thereof, wherein B¹ is

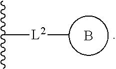

45. The molecule of claim 44, or a pharmaceutically acceptable salt thereof, wherein ring B is an optionally substituted C₃-C₆ cycloalkyl or optionally substituted 4 to 6-membered heterocycloalkyl.

46. The molecule of claim 45, or a pharmaceutically acceptable salt thereof, wherein ring B is an optionally substituted piperidine or an optionally substituted piperazine.

47. The molecule of any one of claims 44-46, or a pharmaceutically acceptable salt thereof, wherein L² is C₁-C₄ alkylene or C₂-C₄ alkynylene.

48. The molecule of claim 47, or a pharmaceutically acceptable salt thereof, wherein L² is C₂-C₄ alkynylene.

49. The molecule of any one of claims 44-46, or a pharmaceutically acceptable salt thereof, wherein L² is absent.

50. The molecule of any one of claims 40-49, or a pharmaceutically acceptable salt thereof, wherein Z is —C(O)—.

51. The molecule of any one of claims 40-49, or a pharmaceutically acceptable salt thereof, wherein Z is absent.

52. The molecule of any one of claims 13-51, or a pharmaceutically acceptable salt thereof, wherein x₁ is 1.

53. The molecule of any one of claims 1-52, or a pharmaceutically acceptable salt thereof, wherein m₁ is 0.

54. The molecule of any one of claims 24-53, or a pharmaceutically acceptable salt thereof, wherein each R^{2d} is independently an optionally substituted C₁-C₁₀ alkyl, optionally substituted C₁-C₁₀ heteroalkyl, optionally substituted C₁-C₁₀ haloalkyl, or optionally substituted PEG₁₋₁₀; each of which is optionally substituted with one or more R¹⁰.

55. The molecule of any one of claims 24-53, or a pharmaceutically acceptable salt thereof, wherein each R^{2a} is independently an optionally substituted C₁-C₁₀ alkyl, which is optionally substituted with one or more R¹⁰.

56. The molecule of any one of claims 1-55, or a pharmaceutically acceptable salt thereof, wherein each R¹⁰ is independently —N₃, —NR^{10a}R^{10b}, —CO(O)R¹⁰, —C(O)OR¹⁰, —C(O)NR^{10a}R^{10b}, —NHC(O)R^{10c}, —NHC(O)OR^{10c}, —OC(O)NR^{10a}R^{10b}, or optionally substituted 5 to 10-membered heteroaryl.

57. The molecule of claim 56, or a pharmaceutically acceptable salt thereof, wherein each R¹⁰ is independently —N₃, —NR^{10a}R^{10b}, —C(O)NR^{10a}R^{10b}, —NHC(O)R^{10c} or optionally substituted 5-membered heteroaryl.

58. The molecule of claim 54 or 55, or a pharmaceutically acceptable salt thereof, wherein R^{2d} is unsubstituted C₁-C₁₀ alkyl.

59. The molecule of claim 58, or a pharmaceutically acceptable salt thereof wherein R^{2d} is methyl.

60. A transcription modulator molecule having a structure of Formula (IV), or a pharmaceutically acceptable salt thereof: 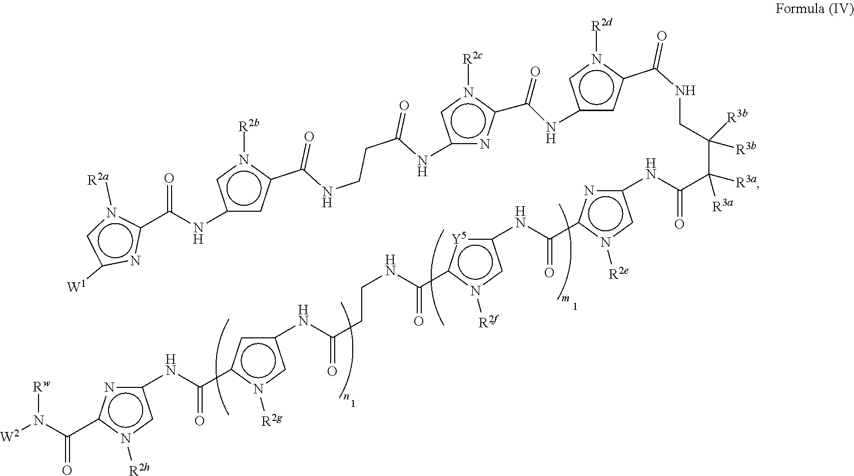
wherein:
W¹ is hydrogen or —N═C(N(R¹⁰)₂)₂, wherein each R¹⁰ is independently hydrogen or C₁-C₃ alkyl;
W² is hydrogen, optionally substituted C₁-C₂₀ alkyl, or optionally substituted C₁-C₂₀ heteroalkyl;
R^{W} is hydrogen or C₁-C₂₀ alkyl;
or W² and R^{W} together with the nitrogen to which they are attached form an optionally substituted 4 to 8-membered heterocycloalkyl which is partially or fully unsaturated;
each Y⁵ is independently N or CH;
each R^{2a}, R^{2b}, R^{2c}, R^{2d}, R^{2e}, R^{2f}, R^{2g}, and R^{2h} is independently hydrogen, optionally substituted C₁-C₅₀ alkyl, optionally substituted C₂-C₅₀ alkenyl, optionally substituted C₂-C₅₀ alkynyl, optionally substituted C₁-C₅₀ heteroalkyl, optionally substituted C₂-C₅₀ heteroalkenyl, optionally substituted C₂-C₅₀ heteroalkynyl, optionally substituted C₁-C₅₀ haloalkyl, optionally substituted C₃-C₈ cycloalkyl, optionally substituted 3 to 8-membered heterocycloalkyl, or optionally substituted PEG₁₋₅₀; each of which is optionally substituted with one or more R¹⁰;
each R^{3a} and R^{3b} is independently hydrogen, halogen, —NR^{11a}R^{11b}, or —NHC(O)R¹², wherein
R^{11a} and R^{11b} are each independently hydrogen, alkyl, or PEG;
R¹² is alkyl, PEG, cycloalkyl, heterocycloalkyl, or phenyl;
or two R^{3a} or two R^{3b} together with the carbon atom to which they are attached form a C₃-C₆ cycloalkyl or 4 to 6-membered heterocycloalkyl;
each R¹⁰ is independently —CN, —OH, —OR^{10a}, —N₃, —NR^{10a}R^{10b}, —CO(O)R^{10c}, —C(O)OR^{10c}, —C(O)NR^{10a}R^{10b}, —NHC(O)R^{10c}, —NHC(O)OR^{10c}, —OC(O)NR^{10a}R^{10b}, or optionally substituted 5 to 10-membered heteroaryl; wherein
R^{10a} and R^{10b} are each independently hydrogen, alkyl, or PEG;
R^{10c} is alkyl, PEG, cycloalkyl, heterocycloalkyl, or phenyl; and
n₁ and m₁ are each independently 0 or 1.

61. The molecule of claim 60, or a pharmaceutically acceptable salt thereof, wherein W² and R^{W} together with the nitrogen to which they are attached form an optionally substituted 4 to 8-membered heterocycloalkyl which is partially or fully unsaturated.

62. The molecule of claim 60, wherein the molecule has the structure of Formula (V), or a pharmaceutically acceptable salt thereof: 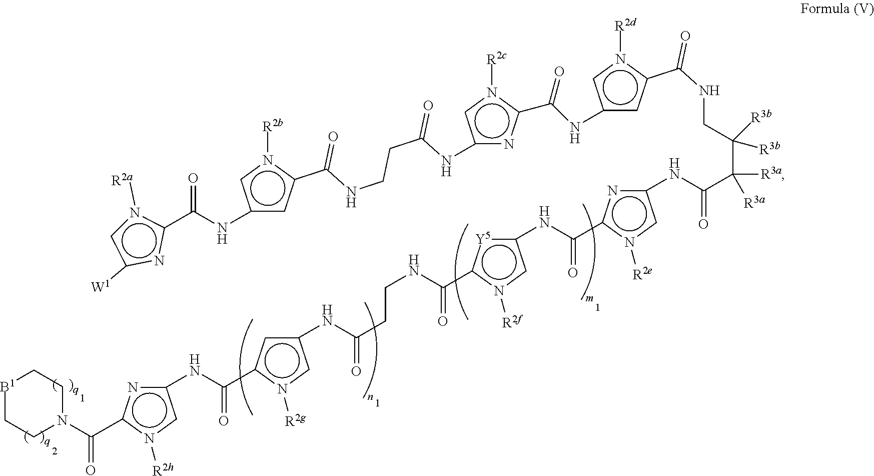
wherein:
B¹ is —CR^{5a}R^{5b}—, —O—, —NR^{5b}—, —S—, —S(O)—, or —S(O)₂—; or B¹ is 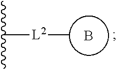
wherein
R^{5a} is hydrogen, —OH, or optionally substituted C₁-C₂₀ alkyl;
R^{5b} is hydrogen, optionally substituted C₁-C₂₀ alkyl, optionally substituted C₂-C₂₀ alkenyl, optionally substituted C₂-C₂₀ alkynyl, optionally substituted C₁-C₂₀ heteroaryl, —C(O)OR⁶, or —C(O)R⁶; or
R^{5a} and R^{5b} together with the nitrogen atom to which they are attached form an optionally substituted 4 to 8-membered heterocycloalkyl;
R⁶ is hydrogen, optionally substituted C₁-C₂₀ alkyl, optionally substituted C₁-C₁₀ haloalkyl, optionally substituted C₃-C₆ cycloalkyl, optionally substituted 3 to 6-membered heterocycloalkyl, or optionally substituted phenyl;
ring B is absent, optionally substituted C₃-C₆ cycloalkyl, optionally substituted 4 to 6-membered heterocycloalkyl, optionally substituted phenyl, or optionally substituted 5 to 10-membered heteroaryl;
L² is absent, C₁-C₄ alkylene, C₂-C₄ alkynelene, or C₂-C₄ alkynylene;
W¹ is hydrogen or —N═C(N(R^{1e})₂)₂, wherein each R^{1e} is independently hydrogen or C₁-C₃ alkyl;
each Y⁵ is independently N or CH;
each R^{2a}, R^{2b}, R^{2c}, R^{2d}, R^{2e}, R^{2f}, R^{2g}, and R^{2h} is independently hydrogen, optionally substituted C₁-C₅₀ alkyl, optionally substituted C₂-C₅₀ alkenyl, optionally substituted C₂-C₅₀ alkynyl, optionally substituted C₁-C₅₀ heteroalkyl, optionally substituted C₂-C₀ heteroalkenyl, optionally substituted C₂-C₅₀ heteroalkynyl, optionally substituted C₁-C₅₀ haloalkyl, optionally substituted C₃-C₈ cycloalkyl, optionally substituted 3 to 8-membered heterocycloalkyl, or optionally substituted PEG₁₋₅₀; each of which is optionally substituted with one or more R¹⁰;
each R^{3a} and R^{3b} is independently hydrogen, halogen, —NR^{11a}R^{11b}, or —NHC(O)R¹², wherein
R^{11a} and R^{11b} are each independently hydrogen, alkyl, or PEG;
R¹² is alkyl, PEG, cycloalkyl, heterocycloalkyl, or phenyl;
or two R^{3a} or two R^{3b} together with the carbon atom to which they are attached form a C₃-C₆ cycloalkyl or 4 to 6-membered heterocycloalkyl;
each R¹⁰ is independently —CN, —OH, —OR^{10a}, —N₃, —NR^{10a}R^{10b}—CO(O)R^{10c}, —C(O)OR^{10c}, —C(O)NR^{10a}R^{10b}, —NHC(O)R^{10c}, —NHC(O)OR^{10c}, —OC(O)NR^{10a}R^{10b}, or optionally substituted 5 to 10-membered heteroaryl: wherein
R^{10a} and R^{10b} are each independently hydrogen, alkyl, or PEG;
R^{10c} is alkyl, PEG, cycloalkyl, heterocycloalkyl, or phenyl;
n₁ and m₁ are each independently 0 or 1; and
q₁ and q₂ are each independently 0-2.

63. The molecule of claim 62, or a pharmaceutically acceptable salt thereof, wherein q₁ is 1 and q₂ is 1.

64. The molecule of claim 62 or 63, or a pharmaceutically acceptable salt thereof, wherein B¹ is —O— or —NR^{5b}—.

65. The molecule of claim 62 or 63, or a pharmaceutically acceptable salt thereof, wherein B¹ is —CR^{5a}R^{5b}—.

66. The molecule of claim 62 or 63, or a pharmaceutically acceptable salt thereof, wherein B¹ is

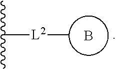

67. The molecule of claim 66, or a pharmaceutically acceptable salt thereof, wherein ring B is an optionally substituted C₃-C₆ cycloalkyl or optionally substituted 4 to 6-membered heterocycloalkyl.

68. The molecule of claim 67, or a pharmaceutically acceptable salt thereof, wherein ring B is an optionally substituted piperidine or an optionally substituted piperazine.

69. The molecule of any one of claims 66-68, or a pharmaceutically acceptable salt thereof, wherein L² is C₁-C₄ alkylene or C₂-C₄ alkynylene.

70. The molecule of any one of claims 66-68, or a pharmaceutically acceptable salt thereof, wherein L² is absent.

71. The molecule of any one of claims 60-70, or a pharmaceutically acceptable salt thereof, wherein each R^{3b} is hydrogen; and each R^{3a} is independently selected from hydrogen, —NR^{11a}R^{11b}, and —NHC(O)R¹².

72. The molecule of any one of claims 60-70, or a pharmaceutically acceptable salt thereof, wherein each R^{3a} is hydrogen; and each R^{3b} is independently selected from hydrogen, —NR^{11a}R^{11b}, and —NHC(O)R¹².

73. The molecule of any one of claims 60-70, or a pharmaceutically acceptable salt thereof, wherein each R^{3a} and each R^{3b} are hydrogen.

74. The molecule of any one of claims 60-70, or a pharmaceutically acceptable salt thereof, wherein two R^{3a} together with the carbon atom to which they are attached form a C₃-C₆ cycloalkyl or 4 to 6-membered heterocycloalkyl; and each R^{3b} is hydrogen.

75. The molecule of any one of claims 60-70, or a pharmaceutically acceptable salt thereof, wherein two R^{3b} together with the carbon atom to which they are attached form a C₃-C₆ cycloalkyl or 4 to 6-membered heterocycloalkyl; and each R^{3a} is hydrogen.

76. The molecule of any one of claims 60-75, or a pharmaceutically acceptable salt thereof, wherein each R^{2a}, R^{2b}, R^{2c}, R^{2d}, R^{2e}, R^{2f}, R^{2g}, and R^{2h} is independently hydrogen, optionally substituted C₁-C₅₀ alkyl, optionally substituted C₁-C₅₀ heteroalkyl, optionally substituted C₁-C₅₀ haloalkyl, or optionally substituted PEG₁₋₅₀; each of which is optionally substituted with one or more R¹⁰.

77. The molecule of any one of claims 60-75, or a pharmaceutically acceptable salt thereof, wherein each R^{2a}, R^{2b}, R^{2c}, R^{2d}, R^{2e}, R^{2f}, R^{2g}, and R^{2h} is independently hydrogen, optionally substituted C₁-C₁₀ alkyl, optionally substituted C₁-C₁₀ heteroalkyl, optionally substituted C₁-C₁₀ haloalkyl, or optionally substituted PEG₁₋₁₀; each of which is optionally substituted with one or more R¹⁰.

78. The molecule of any one of claims 60-75, or a pharmaceutically acceptable salt thereof, wherein each R^{2a}, R^{2b}, R^{2c}, R^{2d}, R^{2e}, R^{2f}, R^{2g}, and R^{2h} is independently C₁-C₁₀ alkyl, each of which is optionally substituted with one or more R¹⁰.

79. The molecule of any one of claims 60-75, or a pharmaceutically acceptable salt thereof, wherein each R^{2a}, R^{2b}, R^{2a}, and R^{2g} is independently an optionally substituted C₁-C₁₀ alkyl, optionally substituted C₁-C₁₀ heteroalkyl, optionally substituted C₁-C₁₀ haloalkyl, or optionally substituted PEG₁₋₁₀; each of which is optionally substituted with one or more R¹⁰; and each of R^{2c}, R^{2e}, and R^{2h} is independently unsubstituted C₁-C₁₀ alkyl.

80. The molecule of claim 79, or a pharmaceutically acceptable salt thereof, wherein each of R^{2c}, R^{2e}, and R^{2h} is methyl.

81. The molecule of claim 79 or 80, or a pharmaceutically acceptable salt thereof, wherein each R^{2a}, R^{2b}, and R^{2g} is independently unsubstituted C₁-C₁₀ alkyl.

82. The molecule of claim 81, or a pharmaceutically acceptable salt thereof, wherein each R^{2a}, R^{2b}, and R^{2g} is methyl.

83. The molecule of claim 79, or a pharmaceutically acceptable salt thereof, wherein each R^{2a} is independently an optionally substituted C₁-C₁₀ alkyl, which is optionally substituted with one or more R¹.

84. The molecule of any one of claims 60-79 or 83, or a pharmaceutically acceptable salt thereof, wherein each R¹⁰ is independently —N₃, —NR^{10a}R^{10b}, —CO(O)R^{10c}, —C(O)OR^{10c}, —C(O)NR^{10a}R^{10b}, —NHC(O)R^{10c}, —NHC(O)OR^{10c}, —OC(O)NR^{10a}R^{10b}, or optionally substituted 5 to 10-membered heteroaryl.

85. The molecule of claim 84, or a pharmaceutically acceptable salt thereof, wherein each R^{10c} is independently —N₃, —NR^{10a}R^{10b}, —C(O)NR^{10a}R^{10b}, —NHC(O)R^{10c}, or optionally substituted 5-membered heteroaryl.

86. The molecule of claim 79, or a pharmaceutically acceptable salt thereof, wherein R^{2d} is unsubstituted C₁-C₁₀ alkyl.

87. The molecule of claim 86, or a pharmaceutically acceptable salt thereof wherein R^{2d} is methyl.

88. The molecule of any one of claims 60-87, or a pharmaceutically acceptable salt thereof, wherein n₁ is 1.

89. The molecule of any one of claims 60-88, or a pharmaceutically acceptable salt thereof, wherein m₁ is 0.

90. The molecule of any one of claims 1-89, or a pharmaceutically acceptable salt thereof, wherein W¹ is hydrogen.

91. A transcription modulator molecule selected from Table 2, or a pharmaceutically acceptable salt thereof.

92. A pharmaceutical composition comprising a molecule of any one of claims 1-91, or a pharmaceutically acceptable salt thereof; and a pharmaceutically acceptable excipient.

93. A method of binding to a nucleotide repeat sequence CAG, the method comprising contacting a molecule of any one of claims 1-91, or a pharmaceutically acceptable salt thereof, with a nucleotide comprising the repeat CAG.
